(19) United States
(12) Patent Application Publication
(10) Pub. No.: US 2010/0234351 A1
(43) Pub. Date: Sep. 16, 2010

(54) FUSED NITROGEN-COMPRISING HETEROCYCLIC COMPOUND

(75) Inventors: Masaki Seto, Osaka-shi (JP); Tomohiro Ohashi, Osaka-shi (JP)

Correspondence Address: HAMRE, SCHUMANN, MUELLER & LARSON, P.C., P.O. BOX 2902, MINNEAPOLIS, MN, 55402-0902, US

(73) Assignee: TAKEDA PHARMACEUTICAL COMPANY LIMITED, Osaka-shi, Osaka (JP)

(21) Appl. No.: 12/280,228

(22) PCT Filed: Feb. 22, 2007
(86) PCT No.: PCT/JP2007/053859

§ 371 Date: Aug. 21, 2008

Related U.S. Application Data
(60) Provisional application No. 60/775,777, filed on Feb. 23, 2006.

(51) Int. Cl. A61K 31/55; C07D 487/04; A61P 35/00; A61K 31/519

(52) U.S. Cl. 514/215; 540/578; 540/476; 514/258.1

(57) ABSTRACT

A compound of the formula:

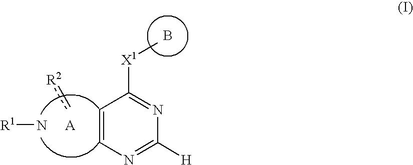

wherein ring A is a 7-membered or 8-membered nitrogen-containing ring optionally further substituted, ring B is an optionally substituted aryl group or an optionally substituted heteroaryl group, X¹ is a group represented by —NR³—Y¹—, —O—, —S—, —SO—, —SO₂— or —CHR³— wherein R³ is a hydrogen atom or an optionally substituted aliphatic hydrocarbon group, or R³ may be bonded to the carbon atom of ring B to form an optionally substituted ring structure, and Y¹ is a bond or an optionally substituted alkylene, R¹ is a hydrogen atom, or an optionally substituted group bonded via a carbon atom or a sulfur atom, the formula [symbol] shows a single bond or a double bond, when [symbol] R² is —R², R² is a hydrogen atom, or an optionally substituted group bonded via a carbon atom, a nitrogen atom, an oxygen atom or a sulfur atom, and when [symbol] R² is ═R², R² is an oxo group, an optionally substituted alkylidene group, or an optionally substituted imino group.

### TECHNICAL FIELD

The present invention relates to a fused pyrimidine compound having a growth factor receptor tyrosine kinase inhibitory activity, which is useful for the prophylaxis or treatment of cancer, a production method thereof and use thereof.

### BACKGROUND OF THE INVENTION

The gene of cell growth factor and growth factor receptor is called a protooncogene and plays a key role in the pathology of human tumor. The epithelial cell growth factor receptor family (erbB) includes EGFR, HER², HER³ and HER4, which are type I receptor type tyrosine kinases. These erbB family express in various cell groups, and are deeply involved in the control of the growth and differentiation of cells and the control of suppression of cell death (apoptosis suppression).

For example, high expression of EGFR and HER², and homeostatic activation of receptors are empirically known to transform cells.

It is also known that high expression and simultaneous expression of each of these receptors are poor prognostic factors in various cancer patients.

These receptors are bound with many peptide ligands such as EGF, TGFα and the like, and binding of the ligand promotes homo- or heterodimerization of the receptors. This induces increase of kinase activity from self-phosphorylation or transphosphorylation of the receptors, and causes activation of downstream signaling pathway (MAPK, Akt) via a protein bound with a particular phosphorylated tyrosine residue. This is the substance of the receptor activity of the above-mentioned cell growth, differentiation, cell death suppression and the like, which is considered to be responsible for the high expression of receptor in cancer and malignant degeneration of cancer due to topical increase in the ligand concentration.

Many cancers are associated with the high expression of EGFR or HER². For example, breast cancer (20-30%), ovarian cancer (20-40%), non-small cell lung cancer (30-60%), colorectal cancer (40-80%), prostate cancer (10-60%), bladder cancer (30-60%), kidney cancer (20-40%) and the like can be mentioned. Moreover, receptor expression and prognosis are correlated, and receptor expression is a poor prognostic factor in breast cancer, non-small cell lung cancer and the like.

In recent years, clinical use of a humanized anti-HER² antibody (Trastuzumab) against HER² highly expressing breast cancer, clinical, trial of anti-EGFR antibody and clinical trials of several low molecular weight receptor enzyme inhibitors have demonstrated a potential of these drugs against HER² or EGFR for therapeutic drugs for cancer. While these drugs show a tumor growth inhibitory action in clinical and non-clinical trials, they are known to induce inhibition of receptor enzyme activity and suppression of downstream signaling pathway. Therefore, a compound inhibiting EGFR or HER² kinase, or inhibiting activation of EGFR or HER² kinase is effective as a therapeutic drug for cancer.

As a compound that inhibits receptor type tyrosine kinases including HER²/EGFR kinase, fused heterocyclic compounds (e.g., WO97/13771, WO98/02437, WO00/44728, WO01/19828, WO2005/118588), quinazoline derivatives (e.g., WO02/02552, WO01/98277, WO03/049740, WO03/050108), thienopyrimidine derivatives (e.g., WO03/053446), aromatic azole derivatives (e.g., WO98/03648, WO01/77107, WO03/031442) and the like are known; however, there is no HER² kinase inhibitory substance to the present that has been marketed as a therapeutic drug for cancer.

### DISCLOSURE OF THE INVENTION

The present invention aims at provision of a compound having a superior tyrosine kinase inhibitory action, which is highly safe and sufficiently satisfactory as a pharmaceutical product.

The present inventors have conducted intensive studies in an attempt to solve the aforementioned problems and found that the compounds represented by the following formula (I) and a salt thereof (sometimes to be referred to as compound (I) in the present specification) have a superior tyrosine kinase inhibitory action. Further studies have resulted in the completion of the present invention.

Accordingly, the present invention relates to the following:

[1] A compound represented by the formula:

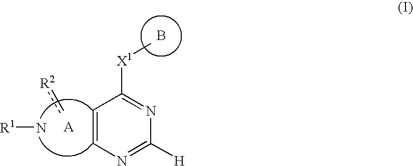

wherein
ring A is an optionally further substituted nitrogen-containing 7-membered or 8-membered ring,
ring B is an optionally substituted aryl group or an optionally substituted heteroaryl group,
X¹ is a group represented by —NR³—Y¹—, —O—, —S—, —SO—, —SO₂— or —CHR³—
wherein
R³ is a hydrogen atom or an optionally substituted aliphatic hydrocarbon group, or R³ is optionally bonded to the carbon atom on ring B to form an optionally substituted ring structure, and
Y¹ is a bond or an optionally substituted C₁₋₄ alkylene,
   R¹ is a hydrogen atom, or an optionally substituted group bonded via a carbon atom or a sulfur atom, formula [symbol] is a single bond or a double bond, and R² is a hydrogen atom, or an optionally substituted group bonded via a carbon atom, a nitrogen atom, an oxygen atom or a sulfur atom when [symbol] R² is —R², and an oxo group, an optionally substituted alkylidene group or an optionally substituted imino group when [symbol] R² is ═R², or
   R¹ and R², or R² and R³ are optionally bonded to each other to form an optionally substituted ring structure, or a salt thereof.
   [2] The compound of the above-mentioned [1], which is a compound represented by the formula:

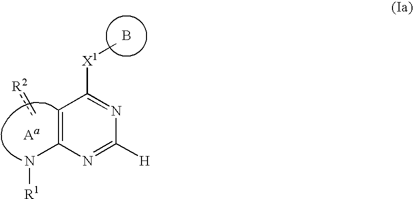

wherein ring A^{a} is an optionally further substituted nitrogen-containing 7-membered or 8-membered ring, and other symbols are as defined in the above-mentioned [1], or a salt thereof.
[3] The compound of the above-mentioned [1], which is a compound represented by the formula:

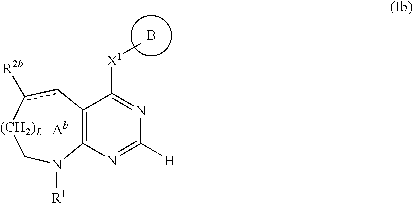

wherein
ring A^{b} is an optionally further substituted nitrogen-containing 7-membered or 8-membered ring,

### L is 1 or 2,

formula [symbol] is a single bond or a double bond,
R^{2b} is a hydrogen atom, or an optionally substituted group bonded via a carbon atom, a nitrogen atom, an oxygen atom or a sulfur atom, and
other symbols are as defined in the above-mentioned [1], or a salt thereof.
[4] The compound of the above-mentioned [1], which is a compound represented by the formula:

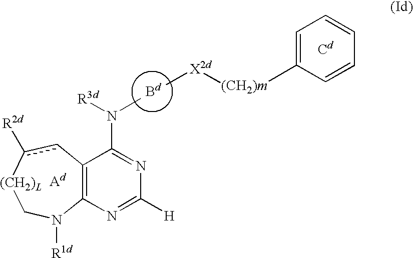

wherein
ring A^{d} is an optionally further substituted nitrogen-containing 7-membered or 8-membered ring,

### L is 1 or 2,

formula [symbol] is a single bond or a double bond,
ring B^{d} is an optionally further substituted phenyl group or an optionally further substituted pyridyl group,
ring C^{d} is an optionally substituted phenyl group,
X^{2d} is a group represented by —O—, —S—, —SO—, —SO₂—, —CH₂— or —CO—NR^{5d}— (wherein R^{5d} is a hydrogen atom, or an optionally substituted C₁₋₆ alkyl group),
m is an integer of 0 to 5,
R^{1d} is a hydrogen atom, or an optionally substituted group bonded via a carbon atom or a sulfur atom,
R^{2d} is a hydrogen atom, or an optionally substituted group bonded via a carbon atom, a nitrogen atom, an oxygen atom or a sulfur atom, and
R^{3d} is a hydrogen atom or an optionally substituted aliphatic hydrocarbon group, or R^{3d} is optionally bonded to the carbon atom on ring B^{d} to form an optionally substituted ring structure,
or a salt thereof.
[5] The compound of the above-mentioned [4], wherein
R^{1d} is a hydrogen atom or a C₁₋₆ alkyl group,

### R^{2d} is

(i) a C₁₋₆ alkyl group substituted by substituents selected from the group consisting of

(a) hydroxy,

(b) —NH—CO—(CH₂)ₚ—SO₂—C₁₋₆ alkyl (p is an integer of 0.1 to 6),

(c) —NH—CO—C₁₋₆ alkyl-hydroxy,

(d) —NH—CO— (CH₂)_{p′}—C₁₋₆ alkoxy-C₁₋₆ alkoxy (p′ is an integer of 1 to 6),

(e) C₁₋₆ alkoxyimino substituted by substituents selected from the group consisting of (1) hydroxy, (2) C₁₋₆ alkoxy, (3) di-C₁₋₆ alkylamino, (4) C₁₋₆ alkylsulfonyl and (5) 5- to 8-membered heterocyclic group containing, besides carbon atoms, 1 to 3 heteroatoms selected from the group consisting of nitrogen atom, oxygen atom and sulfur atom,

(f) C₁₋₆ alkylamino having cyano,

(g) C₁₋₆ alkylamino having halogen atom,

(h) C₁₋₆ alkylamino having hydroxy,

(i) C₁₋₆ alkylamino having C₁₋₆ alkoxy,

(j) C₁₋₆ alkylamino having C₁₋₆ alkylsulfonyl

optionally having hydroxy,

(k) di-C₁₋₆ alkylamino optionally having 1 or 2 substituents selected from the group consisting of (1) hydroxy, (2) cyano, (3) halogen atom and (4) C₁₋₆ alkylsulfonyl,

(l) C₃₋₇ cycloalkylamino optionally having hydroxy,

(m) 5- to 8-membered heterocyclyl-amino containing, besides carbon atoms, 1 to 3 heteroatoms selected from the group consisting of nitrogen atom, oxygen atom and sulfur atom,

(o) 5- to 8-membered cyclic amino optionally having C₁₋₆ alkoxy or a C₁₋₆ alkylsulfonyl,

(p) N—C₁₋₆ alkyl-N—C₃₋₇ cycloalkylamino optionally having C₁₋₆ alkylsulfonyl,

(q) cyano,

(r) C₁₋₆ alkylamino having C₁₋₆ alkoxy optionally having hydroxy or a C₁₋₆ alkoxy, and

(s) 5- to 8-membered heterocycle containing, besides carbon atoms, 1 to 3 heteroatoms selected from the group consisting of nitrogen atom, oxygen atom and sulfur atom, which is optionally substituted by substituents selected from the group consisting of oxo, C₁₋₆ alkylsulfonyl and C₁₋₆ alkoxy,

(ii) a C₂₋₆ alkenyl group having (a) hydroxy, (b) di-C₁₋₆ alkylamino or (c) C₁₋₆ alkoxy-carbonyl,
(iii) a C₁₋₆ alkoxy-carbonyl group,
(iv) a group represented by —CO—NR^{a}R^{b}
wherein

R^{a} is a hydrogen atom or a C₁₋₆ alkyl group, and

R^{b} is
(a) a C₁₋₆ alkyl group optionally substituted by 1 or 2 substituents selected from the group consisting of
   (1) hydroxy,
   (2) amino,
   (3) C₁₋₆ alkylamino having hydroxy,
   (4) C₁₋₆ alkylamino having C₁₋₆ alkoxy,
   (5) cyano,
   (6) amino mono- or di-substituted by C₁₋₆ alkyl optionally having hydroxy,
   (7) C₁₋₆ alkyl-carbonylamino,
   (8) C₁₋₆ alkoxy,
   (9) C₁₋₆ alkoxy having hydroxy,
   (10) C₁₋₆ alkoxy having C₁₋₆ alkoxy,
   (11) C₁₋₆ alkoxy having hydroxy and C₁₋₆ alkoxy,
   (12) C₁₋₆ alkoxy having C₁₋₆ alkylsulfonyl,
   (13) C₁₋₆ alkoxy having cyano,
   (14) C₁₋₆ alkoxy-carbonyl,
   (15) C₁₋₆ alkylsulfonyl optionally having hydroxy or C₁₋₆ alkoxy,
   (16) 5- to 8-membered heterocyclyl-sulfonyl containing, besides carbon atoms, 1 to 3 heteroatoms selected from the group consisting of nitrogen atom, oxygen atom and sulfur atom,
   (17) C₆₋₁₈ arylsulfonyl,
   (18) 5- to 8-membered heterocycle containing, besides carbon atoms, 1 to 3 heteroatoms selected from the group consisting of nitrogen atom, oxygen atom and sulfur atom, which optionally has 1 or 2 substituents selected from the group consisting of hydroxy, C₁₋₆ alkyl, C₆₋₁₈ aryl and C₆₋₁₈ aryl-C₁₋₆ alkyl,
   (19) C₃₋₇ cycloalkyl optionally having hydroxy, and
   (20) C₆₋₁₈ aryl optionally having 1 or 2 halogens,
(b) a C₂₋₆ alkenyl group,
(c) a C₃₋₇ cycloalkyl group optionally having hydroxy,
(d) a C₁₋₆ alkoxy group, or
(e) a 5- to 8-membered heterocyclic group containing, besides carbon atoms, 1 to 3 heteroatoms selected from the group consisting of nitrogen atom, oxygen atom and sulfur atom, which optionally has 1 or 2 substituents selected from the group consisting of hydroxy, C₁₋₆ alkyl, C₆₋₁₈ aryl and C₆₋₁₈ aryl-C₁₋₆ alkyl,
   (v) a 5- to 8-membered cyclic amino-carbonyl group optionally having substituents selected from the group consisting of

(a) hydroxy,

(b) C₁₋₆ alkylsulfonyl, and

(c) C₁₋₆ alkyl optionally having C₁₋₆ alkylsulfonyl,

(vi) a carboxy group,
(vii) an amino group optionally substituted by C₁₋₆ alkoxy-carbonyl optionally having C₁₋₆ alkylsulfonyl, or
(viii) a 5- to 8-membered heterocyclic group containing, besides carbon atoms, 1 to 3 heteroatoms selected from the group consisting of nitrogen atom, oxygen atom and sulfur atom, which optionally has C₁₋₆ alkyl,
R^{3d} is a hydrogen atom,
ring B^{d} is a phenyl group optionally further substituted by C₁₋₆ alkyl group or halogen atom, or a pyridyl group optionally further substituted by C₁₋₆ alkyl group or halogen atom,
ring C^{d} is a phenyl group optionally substituted by substituents selected from the group consisting of
(i) optionally halogenated C₁₋₆ alkyl,
(ii) C₁₋₆ alkoxy optionally having halogen atom or C₃₋₇ cycloalkyl,
(iii) C₁₋₆ alkyl-carbamoyl optionally having hydroxy,
(iv) halogen atom,
(v) cyano,
(vi) C₁₋₆ alkylthio optionally having halogen atom,
(vii) C₁₋₆ alkylsulfinyl optionally having halogen atom, and
(viii) C₁₋₆ alkylsulfonyl optionally having halogen atom or C₃₋₇ cycloalkyl,
X^{2d} is a group represented by —O— or —S—, and
m is 0 or 1.
[6] The compound of the above-mentioned [1], which is a compound represented by the formula:

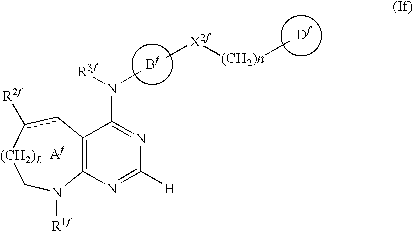

wherein
ring A^{f} is an optionally further substituted nitrogen-containing 7-membered or 8-membered ring,

### L is 1 or 2,

formula [symbol] is a single bond or a double bond,
ring B^{f} is an optionally further substituted phenyl group or an optionally further substituted pyridyl group,
ring D^{f} is an optionally substituted aromatic heterocyclic group,
X^{2f} is a group represented by —O—, —S—, —SO—, —SO₂—, —CH₂— or —CO—NR^{5f}— (wherein R^{5f} is a hydrogen atom, or an optionally substituted C₁₋₆ alkyl group),
n is an integer of 0 to 5,
R^{1f} is a hydrogen atom, or an optionally substituted group bonded via a carbon atom or a sulfur atom,
R^{2f} is a hydrogen atom, or an optionally substituted group bonded via a carbon atom, a nitrogen atom, an oxygen atom or a sulfur atom, and

R^{3f} is a hydrogen atom or an optionally substituted aliphatic hydrocarbon group, or R^{3f} is optionally bonded to the carbon atom on ring B^{f} to form an optionally substituted ring structure,

or a salt thereof.
[7] The compound of the above-mentioned [6], wherein

### L is 1,

R^{1f} is a hydrogen atom or a C₁₋₆ alkyl group,

### R^{2f} is

(i) a C₁₋₆ alkyl group optionally having hydroxy,
(ii) a C₁₋₆ alkoxy-carbonyl group,
(iii) a group represented by —CO—NR^{c}R^{d}

wherein

R^{c} is a hydrogen atom, and

R^{d} is

(a) a C₁₋₆ alkyl group optionally substituted by substituents selected from the group consisting of (1) C₁₋₆ alkoxy, (2) C₁₋₆ alkoxy optionally having substituents selected from the group consisting of (1′) hydroxy, (2′) cyano and (3′) C₁₋₆ alkoxy, and (3) C₁₋₆ alkylsulfonyl optionally having hydroxy,

(b) a C₃₋₇ cycloalkyl group, or

(c) a C₁₋₆ alkoxy group optionally substituted by C₁₋₆ alkylsulfonyl,

(iv) a 5- to 8-membered cyclic amino-carbonyl group optionally substituted by substituents selected from the group consisting of

(a) hydroxy, and

(b) C₁₋₆ alkyl optionally having hydroxy, or

(v) a carboxy group,
R^{3f} is a hydrogen atom,
ring B^{f} is a phenyl group optionally further substituted by C₁₋₆ alkyl group,
X^{f} is a group represented by —O—,
n is 0, and
ring D^{f} is a 5- or 6-membered monocyclic aromatic heterocycle containing, besides carbon atoms, 1 to 3 heteroatoms selected from the group consisting of nitrogen atom, oxygen atom and sulfur atom, which is optionally substituted by C₁₋₆ alkyl group.
[8] The compound of the above-mentioned [1], which is a compound represented by the formula:

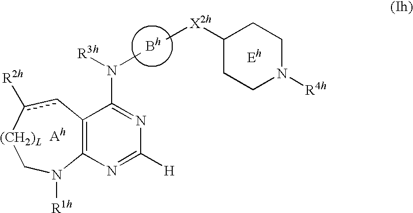

wherein
ring A^{h} is an optionally further substituted nitrogen-containing 7-membered or 8-membered ring,

### L is 1 or 2,

formula [symbol] is a single bond or a double bond,
ring B^{h} is an optionally further substituted phenyl group or an optionally further substituted pyridyl group,
ring E^{h} is an optionally further substituted piperidyl group,
X^{2h} is a group represented by —O—, —S—, —SO—, —SO₂—, —CH₂— or —CO—NR^{5h}— (wherein R^{5h} is a hydrogen atom or an optionally substituted C₁₋₆ alkyl group),
R^{1h} is a hydrogen atom, or an optionally substituted group bonded via a carbon atom or a sulfur atom,
R^{2h} is a hydrogen atom, or an optionally substituted group bonded via a carbon atom, a nitrogen atom, an oxygen atom or a sulfur atom,
R^{3h} is a hydrogen atom, or an optionally substituted aliphatic hydrocarbon group, or R^{3h} is optionally bonded to the carbon atom on ring B^{h} to form an optionally substituted ring structure, and
R^{4h} is a hydrogen atom or an acyl group, or a salt thereof.
[9] The compound of the above-mentioned [8], wherein

### L is 1,

R^{1h} is a hydrogen atom,

### R^{2h} is

(i) a C₁₋₆ alkyl group optionally substituted by hydroxy,
(ii) a C₁₋₆ alkoxy-carbonyl group,
(iii) a group represented by —CO—NR^{e}R^{f}

wherein

R^{e} is a hydrogen atom or a C₁₋₆ alkyl group,

R^{f} is
(a) a C₁₋₆ alkyl group optionally substituted by 1 or 2 substituents selected from the group consisting of
   (1) hydroxy,
   (2) amino,
   (3) cyano,
   (4) amino mono- or di-substituted by C₁₋₆ alkyl optionally having hydroxy,
   (5) C₁₋₆ alkyl-carbonylamino,
   (6) C₁₋₆ alkoxy optionally having hydroxy,
   (7) C₁₋₆ alkoxy-carbonyl,
   (8) C₁₋₆ alkylsulfonyl,
   (9) 5- to 8-membered heterocyclyl-sulfonyl containing, besides carbon atoms, 1 to 3 heteroatoms selected from the group consisting of nitrogen atom, oxygen atom and sulfur atom,
   (10) 5- to 8-membered heterocyclic group containing, besides carbon atoms, 1 to 3 heteroatoms selected from the group consisting of nitrogen atom, oxygen atom and sulfur atom, which optionally has 1 to 2 substituents selected from the group consisting of C₁₋₆ alkyl, C₆₋₁₈ aryl and C₆₋₁₈ aryl-C₁₋₆ alkyl,
   (11) C₆₋₁₈ aryl-sulfonyl, and
   (12) C₆₋₁₈ aryl group optionally having 1 to 2 halogens,
(b) a C₃₋₇ cycloalkyl group,
(c) a C₁₋₆ alkoxy group, or
(d) a 5- to 8-membered heterocyclic group containing, besides carbon atoms, 1 to 3 heteroatoms selected from the group consisting of nitrogen atom, oxygen atom and sulfur atom, which optionally has C₁₋₆ alkyl or C₆₋₁₈ aryl-C₁₋₆ alkyl, or
   (iv) a 5- to 8-membered cyclic amino-carbonyl group optionally substituted by substituents selected from the group consisting of

(a) hydroxy,

(b) C₁₋₆ alkylsulfonyl, and

(c) C₁₋₆ alkyl optionally having C₁₋₆ alkylsulfonyl,

R^{3h} is a hydrogen atom,
ring B^{h} is a phenyl group optionally further substituted by halogen atom,
X^{2h} is a group represented by —O—, and
R^{4h} is (1) a C₃₋₇ cycloalkyl-carbonyl group, (2) a C₁₋₆ alkoxy-carbonyl group, or (3) a 5- to 8-membered heterocyclyl-carbonyl group containing, besides carbon atoms, 1 to 3 heteroatoms selected from the group consisting of nitrogen atom, oxygen atom and sulfur atom, which optionally has 1 or 2 C₁₋₆ alkyls.
[10] A following compound;
4-({3-chloro-4-[3-(trifluoromethyl)phenoxy]phenyl}amino)-N-[3-(1H-imidazol-1-yl)propyl]-8,9-dihydro-7H-pyrimido[4,5-b]azepin-6-carboxamide,
4-({3-chloro-4-[3-(trifluoromethyl)phenoxy]phenyl}amino)-N-[2-(2-hydroxyethoxy)ethyl]-8,9-dihydro-7H-pyrimido[4,5-b]azepin-6-carboxamide,
4-({3-chloro-4-[3-(trifluoromethyl)phenoxy]phenyl}amino)-N-[2-(2-methoxyethoxy)ethyl]-8,9-dihydro-7H-pyrimido[4,5-b]azepin-6-carboxamide,
methyl 4-[(4-{3-[(tert-butylamino)carbonyl]phenoxy}-3-chlorophenyl)amino]-8,9-dihydro-7H-pyrimido[4,5-b]azepine-6-carboxylate,
4-({3-chloro-4-[3-(trifluoromethyl)phenoxy]phenyl}amino)-N-(2,3-dihydroxypropyl)-8,9-dihydro-7H-pyrimido[4,5-b]azepine-6-carboxamide,
4-{[3-chloro-4-(3-chlorophenoxy)phenyl]amino}-N-[2-(2-hydroxyethoxy)ethyl]-8,9-dihydro-7H-pyrimido[4,5-b]azepin-6-carboxamide,
tert-butyl 4-(2-chloro-4-{[6-(hydroxymethyl)-8,9-dihydro-7H-pyrimido[4,5-b]azepin-4-yl]amino}phenoxy)piperidine-1-carboxylate,
4-({3-chloro-4-[3-(trifluoromethyl)phenoxy]phenyl}amino)-N-{2-[(2-hydroxyethyl)sulfonyl]ethyl}-8,9-dihydro-7H-pyrimido[4,5-b]azepin-6-carboxamide,
4-({3-chloro-4-[3-(trifluoromethyl)phenoxy]phenyl}amino)-N-{2-[(2-hydroxyethyl)sulfonyl]-1,1-dimethylethyl}-8,9-dihydro-7H-pyrimido[4,5-b]azepin-6-carboxamide,
N-(tert-butyl)-3-[2-chloro-4-({6-[({2-[(2-hydroxyethyl)sulfonyl]-1,1-dimethylethyl}amino)methyl]-8,9-dihydro-7H-pyrimido[4,5-b]azepin-4-yl}amino)phenoxy]benzamide,
methyl 4-[(6-{3-[(tert-butylamino)carbonyl]phenoxy}-5-chloropyridin-3-yl)amino]-8,9-dihydro-7H-pyrimido[4,5-b]azepin-6-carboxylate,
4-[(3-chloro-4-{3-[(cyclopropylmethyl)sulfonyl]phenoxy}phenyl)amino]-N-{2-[(2-hydroxyethyl)sulfonyl]-1,1-dimethylethyl}-8,9-dihydro-7H-pyrimido[4,5-b]azepin-6-carboxamide,
N-(tert-butyl)-3-{2-chloro-4-[(6-{[(2-hydroxyethoxy)imino]methyl}-8,9-dihydro-7H-pyrimido[4,5-b]azepin-4-yl)amino]phenoxy}benzamide,
N-(tert-butyl)-3-{2-chloro-4-[(6-{[(2-fluoroethyl)amino]methyl}-8,9-dihydro-7H-pyrimido[4,5-b]azepin-4-yl)amino]phenoxy}benzamide,

N-(tert-butyl)-3-(2-chloro-4-{[6-({methyl[2-(methylsulfonyl)ethyl]amino}methyl)-8,9-dihydro-7H-pyrimido[4,5-b]azepin-4-yl]amino}phenoxy)benzamide, or
ethyl (2E)-3-{4-[(4-{3-[(tert-butylamino)carbonyl]phenoxy}-3-chlorophenyl)amino]-8,9-dihydro-7H-pyrimido[4,5-b]azepin-6-yl}acrylate or a salt thereof,
   [11] A prodrug of a compound represented by the formula:

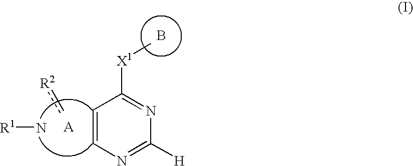

wherein
ring A is an optionally further substituted nitrogen-containing 7-membered or 8-membered ring, ring B is an optionally substituted aryl group or an optionally substituted heteroaryl group,
X¹ is a group represented by —NR³—Y¹—, —O—, —S—, —SO—, —SO₂— or —CHR³—

wherein
R³ is a hydrogen atom or an optionally substituted aliphatic hydrocarbon group, or R³ is optionally bonded to the carbon atom on ring B to form an optionally substituted ring structure, and
Y¹ is a bond or an optionally substituted C₁₋₄ alkylene,
   R¹ is a hydrogen atom, or an optionally substituted group bonded via a carbon atom or a sulfur atom, formula [symbol] is a single bond or a double bond, and
   R² is a hydrogen atom, or an optionally substituted group bonded via a carbon atom, a nitrogen atom, an oxygen atom or a sulfur atom when [symbol] R² is —R², and an oxo group, an optionally substituted alkylidene group or an optionally substituted imino group when [symbol] R² is ═R², or
   R¹ and R², or R² and R³ are optionally bonded to each other to form an optionally substituted ring structure.
   [12] A pharmaceutical agent comprising a compound represented by the formula:

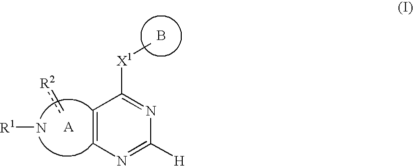

wherein
ring A is an optionally further substituted nitrogen-containing 7-membered or 8-membered ring, ring B is an optionally substituted aryl group or an optionally substituted heteroaryl group,
X¹ is a group represented by —NR³—Y¹—, —O—, —S—, —SO—, —SO₂— or —CHR³—
wherein
R³ is a hydrogen atom or an optionally substituted aliphatic hydrocarbon group, or R³ is optionally bonded to the carbon atom on ring B to form an optionally substituted ring structure, and
Y¹ is a bond or an optionally substituted C₁₋₄ alkylene,
R¹ is a hydrogen atom, or an optionally substituted group bonded via a carbon atom or a sulfur atom, formula [symbol] is a single bond or a double bond, and R² is a hydrogen atom, or an optionally substituted group bonded via a carbon atom, a nitrogen atom an oxygen atom or a sulfur atom when [symbol] R² is —R², and an oxo group, an optionally substituted alkylidene group or an optionally substituted imino group when [symbol] R² is ═R², or
   R¹ and R², or R² and R³ are optionally bonded to each other to form an optionally substituted ring structure, or a salt thereof, or a prodrug thereof.
   [13] The pharmaceutical agent of the above-mentioned [12], which is a tyrosine kinase inhibitor.
   [14] The pharmaceutical agent of the above-mentioned [12], which is an agent for the prophylaxis or treatment of cancer.
   [15] The pharmaceutical agent of the above-mentioned [14], wherein the cancer is breast cancer, ovarian cancer, colorectal cancer, gastric cancer, esophagus cancer, prostate cancer, lung cancer, pancreatic cancer or renal cancer.
   [16] A method for the prophylaxis or treatment of cancer, which comprises administering an effective amount of a compound represented by the formula:

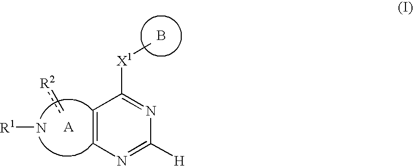

wherein
ring A is an optionally further substituted nitrogen-containing 7-membered or 8-membered ring, ring B is an optionally substituted aryl group or an optionally substituted heteroaryl group,
X¹ is a group represented by —NR³—Y¹—, —O—, —S—, —SO—, —SO₂— or —CHR³—
wherein
R³ is a hydrogen atom or an optionally substituted aliphatic hydrocarbon group, or R³ is optionally bonded to the carbon atom on ring B to form an optionally substituted ring structure, and
Y¹ is a bond or an optionally substituted C₁₋₄ alkylene,
   R¹ is a hydrogen atom, or an optionally substituted group bonded via a carbon atom or a sulfur atom, formula [symbol] is a single bond or a double bond, and
   R² is a hydrogen atom, or an optionally substituted group bonded via a carbon atom, a nitrogen atom an oxygen atom or a sulfur atom when [symbol] R² is —R², and an oxo group, an optionally substituted alkylidene group or an optionally substituted imino group when [symbol] R² is ═R², or
   R¹ and R², or R² and R³ are optionally bonded to each other to form an optionally substituted ring structure, or a salt thereof, or a prodrug thereof, to a mammal.
   [17] Use of a compound represented by the formula:

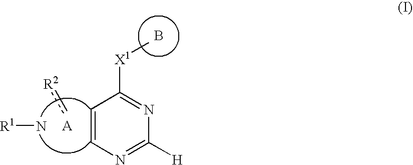

wherein
ring A is an optionally further substituted nitrogen-containing 7-membered or 8-membered ring,
ring B′ is an optionally substituted aryl group or an optionally substituted heteroaryl group,
X¹ is a group represented by —NR³—Y¹—, —O—, —S—, —SO—, —SO₂— or —CHR³—
wherein
R³ is a hydrogen atom or an optionally substituted aliphatic hydrocarbon group, or R³ is optionally bonded to the carbon atom on ring B to form an optionally substituted ring structure, and
Y¹ is a bond or an optionally substituted C₁₋₄ alkylene,
   R¹ is a hydrogen atom, or an optionally substituted group bonded via a carbon atom or a sulfur atom, formula [symbol] is a single bond or a double bond, and
   R² is a hydrogen atom, or an optionally substituted group bonded via a carbon atom, a nitrogen atom, an oxygen atom or a sulfur atom when [symbol] R² is —R², and an oxo group, an optionally substituted alkylidene group or an optionally substituted imino group when [symbol] R² is ═R², or
   R¹ and R², or R² and R³ are optionally bonded to each other to form an optionally substituted ring structure, or a salt thereof, or a prodrug thereof, for producing an agent for the prophylaxis or treatment of cancer.
   [18] A production method of the compound of the above-mentioned [1] or a salt thereof, which comprises subjecting a compound represented by the formula

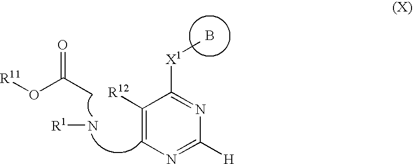

wherein R¹¹ is a C₁₋₆ alkyl group, R¹² is a group represented by —CHO or —COOR¹³ (wherein R¹³ is a C₁₋₆ alkyl group.), and other symbols are as defined in the above-mentioned [1],
or a salt thereof, to an intramolecular dehydrating condensation reaction in the presence of a base, and, as necessary, subjecting the resulting compound to a substituent conversion reaction.
[19] A production method of the compound of the above-mentioned [1] or a salt thereof, which comprises reacting a compound represented by the formula:

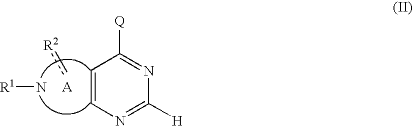

wherein Q is a leaving group, and other symbols are as defined in the above-mentioned [1],
or a salt thereof and a compound represented by the formula:

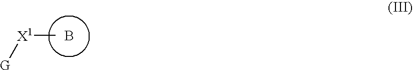

wherein G is a hydrogen atom or a metal atom, and other symbols are as defined in the above-mentioned [1], or a salt thereof.
[20] The compound of the above-mentioned [1], which is a compound represented by the formula:

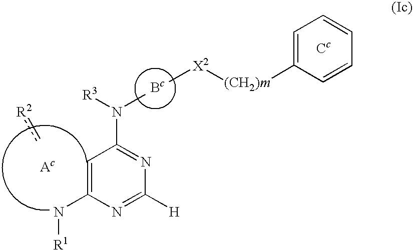

wherein
ring A^{c} is an optionally further substituted nitrogen-containing 7-membered or 8-membered ring,
ring B^{c} is an optionally further substituted phenyl group or an optionally further substituted pyridyl group,
ring C^{c} is an optionally substituted phenyl group,
X² is a group represented by —O—, —S—, —SO—, —SO₂—, —CH₂— or —CO—NR⁵— (wherein R⁵ is a hydrogen atom or an optionally substituted C₁₋₆ alkyl group),
m is an integer of 0 to 5,
other symbols are as defined in the above-mentioned [1], and
R³ is optionally bonded to the carbon atom on ring B^{c} to form an optionally substituted ring structure, or a salt thereof.
[21], The compound of the above-mentioned [1], which is a compound represented by the formula:

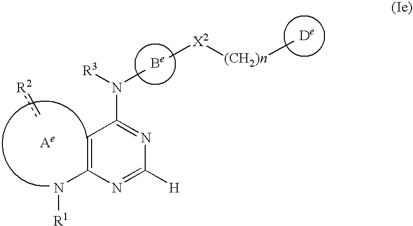

wherein
ring A^{e} is an optionally further substituted nitrogen-containing 7-membered or 8-membered ring,
ring B^{e} is an optionally further substituted phenyl group or an optionally further substituted pyridyl group,
ring D^{e} is an optionally substituted aromatic heterocyclic group,
X² is a group represented by —O—, —S—, —SO—, —SO₂—, —CH₂— or —CO—NR⁵— (wherein R⁵ is a hydrogen atom, or an optionally substituted C₁₋₆ alkyl group),
n is an integer of 0 to 5,
other symbols are as defined in the above-mentioned [1], and
R³ is optionally bonded to the carbon atom on ring B^{e} to form an optionally substituted ring structure, or a salt thereof.
[22] The compound of the above-mentioned [1], which is a compound represented by the formula:

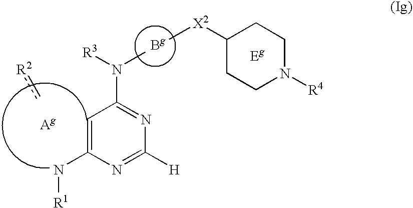

wherein
ring A^{g} is an optionally further substituted nitrogen-containing 7-membered or 8-membered ring,
ring B^{g} is an optionally further substituted phenyl group or an optionally further substituted pyridyl group,
ring E^{g} is an optionally further substituted piperidyl group,
R⁴ is a hydrogen atom or an acyl group,
X² is a group represented by —O—, —S—, —SO—, —SO₂, —CH₂— or —CO—NR⁵— (wherein R⁵ is a hydrogen atom or an optionally substituted C₁₋₆ alkyl group),
other symbols are as defined in the above-mentioned [1], and
R³ is optionally bonded to the carbon atom on ring B^{g} to form an optionally substituted ring structure, or a salt thereof.
[23] The compound of the above-mentioned [1], wherein R¹ is a hydrogen atom or a C₁₋₆ alkyl group.

[24] The compound of the above-mentioned [1], wherein R¹ is a hydrogen atom.

[25] The compound of the above-mentioned [1], wherein R² is an optionally substituted hydrocarbon group, an acyl group or an amino group optionally substituted by acyl group.
[26] The compound of the above-mentioned [1], wherein R³ is a hydrogen atom or a C₁₋₆ alkyl group.
[27] The compound of the above-mentioned [3], wherein R^{2b} is
(i) a C₁₋₆ alkyl group optionally substituted by substituents selected from the group consisting of

(a) hydroxy,

(b) —NH—CO—(CH₂)ₚ—SO₂—C₁₋₆ alkyl (p is an integer of 1 to 6), and

(c) —NH—CO—C₁₋₆ alkyl-hydroxy,

(ii) a C₁₋₆ alkoxy-carbonyl group,
(iii) a group represented by —CO—NR^{a0}R^{b0}
wherein
R^{a0} is a hydrogen atom or a C₁₋₆ alkyl group,
R^{b0} is
(a) a C₁₋₆ alkyl group optionally substituted by 1 or 2 substituents selected from the group consisting of
   (1) hydroxy,
   (2) amino,
   (3) cyano,
   (4) amino mono- or di-substituted by a C₁₋₆ alkyl optionally having hydroxy,
   (5) C₁₋₆ alkylcarbonyl-amino,
   (6) C₁₋₆ alkoxy optionally having hydroxy,
   (7) C₁₋₆ alkoxy having C₁₋₆ alkoxy,
   (8) C₁₋₆ alkoxy having C₁₋₆ alkylsulfonyl,
   (9) C₁₋₆ alkoxy having cyano,
   (10) C₁₋₆ alkoxy-carbonyl,
   (11) C₁₋₆ alkylsulfonyl optionally having hydroxy or C₁₋₆ alkoxy,
   (12) 5- to 8-membered heterocyclyl-sulfonyl containing, besides carbon atoms, 1 to 3 heteroatoms selected from the group consisting of nitrogen atom, oxygen atom and sulfur atom,
   (13) C₆₋₁₈ aryl-sulfonyl,
   (14) 5- to 8-membered heterocyclic group containing, besides carbon atoms, 1 to 3 heteroatoms selected from the group consisting of nitrogen atom, oxygen atom and sulfur atom, which optionally has 1 to 2 substituents selected from the group consisting of hydroxy, C₁₋₆ alkyl, C₆₋₁₈ aryl and C₆₋₁₈ aryl-C₁₋₆ alkyl,
   (15) C₃₋₇ cycloalkyl optionally having hydroxy, and
   (16) C₆₋₁₈ aryl group optionally having halogen,
(b) a C₂₋₆ alkenyl group,
(c) a C₃₋₇ cycloalkyl group optionally having hydroxy,
(d) a C₁₋₆ alkoxy group, or
(e) a 5- to 8-membered heterocyclic group containing, besides carbon atoms, 1 to 3 heteroatoms selected from the group consisting of nitrogen atom, oxygen atom and sulfur atom, which optionally has C₁₋₆ alkyl or C₆₋₁₈ aryl-C₁₋₆ alkyl,
   (iv) a 5- to 8-membered cyclic amino-carbonyl group optionally substituted by substituents selected from the group consisting of

(a) hydroxy,

(b) C₁₋₆ alkylsulfonyl, and

(c) C₁₋₆ alkyl optionally having hydroxy or C₁₋₆ alkylsulfonyl,

(v) a carboxy group, or
(vi) an amino group optionally substituted by C₁₋₆ alkoxy-carbonyl optionally having C₁₋₆ alkylsulfonyl.
[28] The compound of the above-mentioned [1], which is a compound represented by the formula:

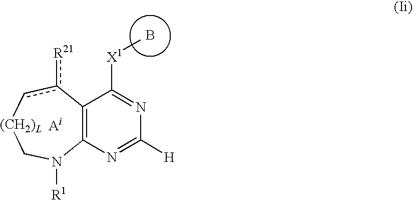

wherein
ring Aⁱ is an optionally further substituted nitrogen-containing 7-membered or 8-membered ring,

### L is 1 or 2,

formula [symbol] is a single bond or a double bond,
R²ⁱ is a hydrogen atom, or an optionally substituted group bonded via a carbon atom, a nitrogen atom, an oxygen atom or a sulfur atom when [symbol] R²ⁱ is —R²ⁱ, and an oxo group, an optionally substituted alkylidene group or an optionally substituted imino group when [symbol] R²ⁱ is ═R²ⁱ,
other symbols are as defined in the above-mentioned [1], and
R²ⁱ and R³ are optionally bonded to each other to form an optionally substituted ring structure,
or a salt thereof.

The present invention can provide a compound having a superior tyrosine kinase inhibitory action, which is low toxic and sufficiently satisfactory as a pharmaceutical product, a production method thereof and use thereof.

Each symbol used in the present specification is described in detail in the following.

In the present specification, unless otherwise specified, as the “halogen atom” (and “halogen” in substituent), fluorine atom, chlorine atom, bromine atom and iodine atom can be mentioned.

In the present specification, unless otherwise specified, as the “alkyl group”, a straight chain or branched alkyl group having 1 to 10 carbon atoms, for example, methyl, ethyl, propyl, isopropyl, butyl, isobutyl, sec-butyl, tert-butyl, pentyl, isopentyl, neopentyl, 1-ethylpropyl, hexyl, isohexyl, 1,1-dimethylbutyl, 2,2-dimethylbutyl, 3,3-dimethylbutyl, 2-ethylbutyl, heptyl, octyl, nonyl, decyl and the like can be mentioned.

In the present specification, unless otherwise specified, as the “C₁₋₈ alkyl group”, for example, methyl, ethyl, propyl, isopropyl, butyl, isobutyl, sec-butyl, tert-butyl, pentyl, isopentyl, neopentyl, 1-ethylpropyl, hexyl, isohexyl, 1,1-dimethylbutyl, 2,2-dimethylbutyl, 3,3-dimethylbutyl, 2-ethylbutyl, heptyl, octyl and the like can be mentioned.

In the present specification, unless otherwise specified, as the “C₁₋₆ alkyl group”, for example, methyl, ethyl, propyl, isopropyl, butyl, isobutyl, sec-butyl, tert-butyl, pentyl, isopentyl, neopentyl, 1-ethylpropyl, hexyl, isohexyl, 1,1-dimethylbutyl, 2,2-dimethylbutyl, 3,3-dimethylbutyl, 2-ethylbutyl and the like can be mentioned.

In the present specification, unless otherwise specified, as the “C₁₋₄ alkyl group”, for example, methyl, ethyl, propyl, isopropyl, butyl, isobutyl, sec-butyl, tert-butyl can be mentioned.

In the present specification, unless otherwise specified, as the “alkenyl group”, an alkenyl group having 2 to 10 carbon atoms, for example, ethenyl, 1-propenyl, 2-propenyl, 2-methyl-1-propenyl, 1-butenyl, 2-butenyl, 3-butenyl, 3-methyl-2-butenyl, 1-pentenyl, 2-pentenyl, 3-pentenyl, 4-pentenyl, 4-methyl-3-pentenyl, 1-hexenyl, 3-hexenyl, 5-hexenyl, 1-heptenyl, 1-octenyl and the like can be mentioned. Of these, C₂₋₆ alkenyl group is preferable.

In the present specification, unless otherwise specified, as the “C₂₋₄ alkenyl group”, for example, ethenyl, 1-propenyl, 2-propenyl, 2-methyl-1-propenyl, 1-butenyl, 2-butenyl, 3-butenyl and the like can be mentioned.

In the present specification, unless otherwise specified, as the “alkynyl group”, an alkynyl group having 2 to 10 carbon atoms, for example, ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl, 1-heptynyl, 1-octynyl and the like can be mentioned. Of these, C₂₋₆ alkynyl group is preferable.

In the present specification, unless otherwise specified, as the “C₂₋₄ alkynyl group”, for example, ethynyl, 1-propynyl, 2-propynyl, 1-butynyl, 2-butynyl, 3-butynyl and the like can be mentioned.

In the present specification, unless otherwise specified, as the “cycloalkyl group”, a cycloalkyl group having 3 to 10 carbon atoms, for example, cyclopropyl, cyclobutyl, cyclopentyl, cyclohexyl, cycloheptyl, cyclooctyl, bicyclo[2.2.1]heptyl, bicyclo[2.2.2]octyl, bicyclo[3.2.1]octyl, bicyclo[3.2.2]nonyl, bicyclo[3.3.1]nonyl, bicyclo[4.2.1]nonyl, bicyclo[4.3.1]decyl, adamantyl and the like can be mentioned. Of these, C₃₋₇ cycloalkyl group is preferable.

In the present specification, unless otherwise specified, as the “C₅₋₈ cycloalkyl group”, for example, cyclopentyl, cyclohexyl, cycloheptyl and cyclooctyl can be mentioned.

In the present specification, unless otherwise specified, as the “C₃₋₇ cycloalkyl group”, for example, cyclopropyl, cyclobutyl, cyclopentyl, cyclohexyl and cycloheptyl can be mentioned.

In the present specification, unless otherwise specified, as the “cycloalkenyl group”, a cycloalkenyl group having 3 to 10 carbon atoms, for example, 2-cyclopenten-1-yl, 3-cyclopenten-1-yl, 2-cyclohexen-1-yl, 3-cyclohexen-1-yl and the like can be mentioned.

In the present specification, unless otherwise specified, as the “cycloalkadienyl group”, a cycloalkadienyl group having 4 to 10 carbon atoms, for example, 2,4-cyclopentadien-1-yl, 2,4-cyclohexadien-1-yl, 2,5-cyclohexadien-1-yl and the like can be mentioned. Of these, C₅₋₇ cycloalkadiene group is preferable.

In the present specification, unless otherwise specified, the term “aryl group” encompasses a monocyclic aryl group and a fused polycyclic aryl group. As the “aryl group”, an aryl group having 6 to 18-carbon atoms, for example, phenyl, naphthyl, anthryl, phenanthryl, acenaphthylenyl, biphenylyl and the like can be mentioned.

In the present specification, unless otherwise specified, as the “C₆₋₁₄ aryl group”, for example, phenyl, naphthyl, anthryl, phenanthryl, acenaphthylenyl, biphenylyl and the like can be mentioned.

In the present specification, unless otherwise specified, as the “aralkyl group”, an aralkyl group having 7 to 15 carbon atoms, for example, benzyl, phenethyl, phenylpropyl, naphthylmethyl and biphenylylmethyl can be mentioned.

In the present specification, unless otherwise specified, as the “alkanoyl group”, an alkanoyl group having 1 to 7 carbon atoms, for example, formyl and C₁₋₆ alkyl-carbonyl (e.g., acetyl, propionyl, butyryl, valeryl and pivaloyl) can be mentioned.

In the present specification, unless otherwise specified, as the “C₁₋₆ alkoxy group”, for example, methoxy, ethoxy, n-propoxy, isopropoxy, n-butoxy, tert-butoxy, n-pentyloxy and n-hexyloxy and the like can be mentioned.

In the present specification, unless otherwise specified, as the “C₁₋₄ alkylene”, for example, —CH₂—, —CH₂CH₂—, —(CH₂)₃—, —(CH₂)₄, —CH(CH₃)—, —C(CH₃)₂—, —CH(CH₃)CH₂—, —CH₂CH(CH₃)—, —C(CH₃)₂CH₂— and —CH₂C(CH₃)₂— can be mentioned.

In the present specification, unless otherwise specified, as the “C₁₋₃ alkylenedioxy group”, for example, methylenedioxy, ethylenedioxy, propylenedioxy and the like can be mentioned.

In the present specification, unless otherwise specified, as the “alkylidene group” of the “optionally substituted alkylidene group”, an alkylidene group having 1 to 10 carbon atoms can be mentioned. Of these, a C₁₋₆ alkylidene group (e.g., methylidene, ethylidene, propylidene, isopropylidene, butenylidene and the like) is preferable. The “alkylidene group” is, for example, optionally substituted by not less than 1 (preferably 1 to 5, more preferably 1 to 3) substituent selected from the below-mentioned substituent group U.

In the present specification, unless otherwise specified, as the “hydrocarbon group” of the “optionally substituted hydrocarbon group”, for example, an alkyl group, an alkenyl group, an alkynyl group, a cycloalkyl group, a cycloalkenyl group, a cycloalkadienyl group, an aryl group, an aralkyl group, an arylalkenyl group, a cycloalkyl-alkyl group and the like can be mentioned. Of these, a C₁₋₁₀ alkyl group, a C₂₋₁₀ alkenyl group, a C₂₋₁₀ alkynyl group, a C₃₋₁₀ cycloalkyl group, a C₃₋₁₀ cycloalkenyl group, a C₄₋₁₀ cycloalkadienyl group, a C₆₋₁₄ aryl group, a C₇₋₁₅ aralkyl group, a C₈₋₁₃ arylalkenyl group, a C₃₋₁₀ cycloalkyl-C₁₋₆ alkyl group and the like preferable.

The above-mentioned C₃₋₁₀ cycloalkyl group, C₃₋₁₀ cycloalkenyl group and C₄₋₁₀ cycloalkadienyl group may be condensed with a benzene ring, and as such fused ring groups, for example, indanyl, dihydronaphthyl, tetrahydronaphthyl, fluorenyl and the like can be mentioned. As the above-mentioned hydrocarbon group, a crosslinking hydrocarbon group such as norbornanyl, adamantyl and the like, and the like can be mentioned.

As the C₈₋₁₃ arylalkenyl group, for example, styryl and the like can be mentioned.

As the C₃₋₁₀ cycloalkyl-C₁₋₆ alkyl group, for example, cyclopropylmethyl, cyclohexylmethyl and the like can be mentioned.

As the above-mentioned “hydrocarbon group”, for example, a chain hydrocarbon group such as a C₁₋₁₀ alkyl group, a C₂₋₁₀ alkenyl group, a C₂₋₁₀ alkynyl group and the like optionally has 1 to 3 substituents at substitutable position(s).

As such substituents, for example,

(1) a C₃₋₁₀ cycloalkyl group optionally substituted by 1 to 3 substituents selected from the group consisting of

(1-1) halogen atom;

(1-2) hydroxy;

(1-3) carboxyl;

(1-4) sulfo;

(1-5) cyano;

(1-6) azido;

(1-7) nitro;

(1-8) nitroso;

(1-9) optionally halogenated C₁₋₄ alkyl;

(1-10) optionally halogenated C₂₋₄ alkenyl;

(1-11) optionally halogenated C₂₋₄ alkynyl;

(1-12) C₃₋₇ cycloalkyl;

(1-13) C₆₋₁₈ aryl;

(1-14) C₆₋₁₈ aryl-C₁₋₆ alkyl;

(1-15) formyl;

(1-16) optionally halogenated C₁₋₆ alkyl-carbonyl;

(1-17) optionally halogenated C₁₋₆ alkoxy-carbonyl;

(1-18) optionally halogenated C₁₋₆ alkylsulfonyl;

(1-19) carbamoyl;

(1-20) carbamoyl mono- or di-substituted by optionally halogenated C₁₋₆ alkyl group;

(1-21) mono- or di-C₆₋₁₄ arylcarbamoyl;

(1-22) thiocarbamoyl optionally mono- or di-substituted by optionally halogenated C₁₋₆ alkyl;

(1-23) ureido optionally mono- or di-substituted by optionally halogenated C₁₋₆ alkyl;

(1-24) mono or di-C₆₋₁₄ arylureido;

(1-25) sulfamoyl optionally mono- or di-substituted by optionally halogenated C₁₋₆ alkyl;

(1-26) optionally halogenated C₁₋₆ alkoxy;

(1-27) optionally halogenated C₂₋₆ alkenyloxy;

(1-28) C₃₋₁₀ cycloalkyloxy;

(1-29) C₇₋₁₅ aralkyloxy;

(1-30) C₆₋₁₄ aryloxy;

(1-31) C₁₋₆ alkyl-carbonyloxy;

(1-32) C₃₋₁₀ cycloalkyl-C₁₋₆ alkyloxy;

(1-33) C₁₋₆ alkylsulfonyloxy;

(1-34) thiol;

(1-35) optionally halogenated C₁₋₆ alkylthio;

(1-36) C₇₋₁₅ aralkylthio;

(1-37) C₆₋₁₄ arylthio;

(1-38) C₁₋₆ alkylsulfinyl;

(1-39) oxo;

(1-40) C₁₋₃ alkylenedioxy;

(1-41) hydroxyimino optionally substituted by C₁₋₆ alkyl; (hereinafter the substituents of the above-mentioned (1-1)-(1-41) are sometimes collectively referred to as “substituent group S”);

(2) a C₆₋₁₈ aryl group optionally substituted by 1 to 3 substituents selected from the above-mentioned substituent group S;
(3) a heterocyclic group optionally substituted by 1 to 3 substituents selected from the above-mentioned substituent group S;
(4) an amino group optionally substituted by 1 or 2 substituents selected from the group consisting of

(4-1) C₁₋₆ alkyl optionally substituted by substituents selected from the group consisting of halogen atom, hydroxy, cyano, C₃₋₇ cycloalkyl, C₁₋₆ alkylsulfonyl, C₁₋₆ alkoxy, hydroxy-C₁₋₆ alkoxy, hydroxy-C₁₋₆ alkylsulfonyl, C₁₋₆ alkoxy-C₁₋₆ alkoxy and the like;

(4-2) optionally halogenated C₂₋₄ alkenyl;

(4-3) optionally halogenated C₂₋₄ alkynyl;

(4-4) C₃₋₇ cycloalkyl optionally substituted by substituents selected from the group consisting of halogen atom, hydroxy, C₃₋₇ cycloalkyl, C₁₋₆ alkylsulfonyl, C₁₋₆ alkoxy and the like;

(4-5) C₆₋₁₄ aryl;

(4-6) C₇₋₁₅ aralkyl;

(4-7) 5- to 8-membered heterocycle containing, besides carbon atoms, 1 to 3 heteroatoms selected from the group consisting of nitrogen atom, oxygen atom and sulfur atom;

(4-8) formyl;

(4-9) C₁₋₆ alkyl-carbonyl optionally substituted by substituents selected from the group consisting of halogen atom, hydroxy, C₃₋₇ cycloalkyl, C₁₋₆ alkylsulfonyl, C₁₋₆ alkoxy, C₁₋₆ alkoxy-C₁₋₆ alkoxy and the like;

(4-10) C₁₋₆ alkoxy-carbonyl optionally substituted by C₁₋₆ alkylsulfonyl;

(4-11) C₆₋₁₄ aryl-carbonyl;

(4-12) C₇₋₁₅ aralkyl-carbonyl;

(4-13) C₃₋₇ cycloalkyl-carbonyl;

(4-14) C₁₋₆ alkyl-carbamoyl;

(4-15) C₆₋₁₄ aryl-carbamoyl;

(4-16) C₇₋₁₅ aralkyl-carbamoyl;

(4-17) C₁₋₆ alkylsulfonyl;

(4-18) C₆₋₁₄ arylsulfonyl;

(4-19) C₇₋₁₅ aralkylsulfonyl;

(4-20) C₁₋₆ alkoxy optionally substituted by substituents selected from the group consisting of halogen atom, hydroxy, C₃₋₇ cycloalkyl, C₁₋₆ alkylsulfonyl, C₁₋₆ alkoxy, 5- to 8-membered heterocyclic group optionally substituted by 1 to 3 substituents selected from the above-mentioned substituent group S, containing, besides carbon atoms, 1 to 3 heteroatoms selected from the group consisting of nitrogen atom, oxygen atom and sulfur atom, and the like; (hereinafter the substituents of the above-mentioned (4-1)-(4-20) are sometimes collectively referred to as “substituent group T”);

(5) an amidino group;
(6) a formyl group or an optionally halogenated C₁₋₆ alkyl-carbonyl group;
(7) an optionally halogenated C₁₋₆ alkoxy-carbonyl group;
(8) a C₁₋₆ alkylsulfonyl group optionally substituted by 1 to 3 substituents selected from the group consisting of a halogen atom, a hydroxy group, a C₁₋₆ alkoxy group;
(9) a carbamoyl group optionally substituted by 1 or 2 substituents selected from substituent group T;
(10) a thiocarbamoyl group optionally mono- or di-substituted by optionally halogenated C₁₋₆ alkyl group;
(11) an ureido group optionally substituted by 1 or 2 substituents selected from substituent group T;
(12) a sulfamoyl group optionally substituted by 1 or 2 substituents selected from substituent group T;
(13) a carboxyl group;
(14) a hydroxy group;
(15) a C₁₋₆ alkoxy group optionally substituted by 1 to 3 substituents selected from the group consisting of halogen atom, cyano group, hydroxy group, carboxyl group, C₁₋₆ alkylsulfonyl group, C₁₋₆ alkoxy group and C₁₋₆ alkoxy-carbonyl group;
(16) an optionally halogenated C₂₋₆ alkenyloxy group;
(17) a C₃₋₁₀ cycloalkyloxy group optionally substituted by 1 to 3 substituents selected from the above-mentioned substituent group S;
(18) a C₇₋₁₅ aralkyloxy group optionally substituted by 1 to 3 substituents selected from the above-mentioned substituent S group;
(19) a C₆₋₁₄ aryloxy group optionally substituted by 1 to 3 substituents selected from the above-mentioned substituent group S;
(20) a C₁₋₆ alkyl-carbonyloxy group;
(21) a thiol group;
(22) an optionally halogenated C₁₋₆ alkylthio group;
(23) a C₇₋₁₅ aralkylthio group optionally substituted by 1 to 3 substituents selected from the above-mentioned substituent group S;
(24) a C₆₋₁₄ arylthio group optionally substituted by 1 to 3 substituents selected from the above-mentioned substituent group S;
(25) a sulfo group;
(26) a cyano group;
(27) an azido group;
(28) a nitro group;
(29) a nitroso group;
(30) a halogen atom;
(31) a C₁₋₆ alkylsulfinyl group;
(32) an oxo group;
(33) a C₃₋₁₀ cycloalkyl-C₁₋₆ alkyloxy group;
(34) a C₁₋₃ alkylenedioxy group;
(35) a hydroxyimino group optionally substituted by a C₁₋₆ alkyl group;
(36) a 5- to 8-membered heterocycle optionally substituted by 1 to 3 substituents selected from the above-mentioned substituent group S, containing, besides carbon atoms, 1 to 3 heteroatoms selected from the group consisting of nitrogen atom, oxygen atom and sulfur atom-sulfonyl group;
(37) a C₆₋₁₈ aryl-sulfonyl group optionally substituted by 1 to 3 substituents selected from the above-mentioned substituent group S;
(38) a group represented by the formula: —(CH₂)ᵤ—R^{x};
(39) a group represented by the formula: —(CH₂)ᵤ-Z^{1x}-optionally halogenated C₁₋₄ alkyl;
(40) a group represented by the formula: —(CH₂)ᵤ-Z^{1x}-C₃₋₇ cycloalkyl;
(41) a group represented by the formula: —(CH₂)ᵤ-Z^{2x}-(CH₂)ᵥ—R^{x};
(42) a group represented by the formula: —(CH₂)ᵤ-Z^{2x}-(CH₂)ᵥ-Z^{1x}-optionally halogenated C₁₋₄ alkyl;
(43) a group represented by the formula: —(CH₂)ᵤ-Z^{2x}-(CH₂)ᵥ-Z^{1x}-C₃₋₇ cycloalkyl;
(44) a group represented by the formula: —(CH₂)ᵤ-Z^{1x}-heterocycle optionally substituted by 1 to 3 substituents selected from substituent group S;
(45) a group represented by the formula: —(CH₂)ᵤ-Z^{2x}-C₁₋₄ alkoxy;
(46) a group represented by the formula: —(CH₂)ᵤ-Z^{2x}-(CH₂)ᵥ-Z^{1x}-(CH₂)ᵥ-Z^{1x}-C₁₋₄ alkyl;
(47) an imino group optionally substituted by substituents selected from substituent group T;
(in the present specification, the substituents of the above-mentioned (1)-(47) are sometimes referred to as “substituent group U”) and the like can be mentioned. When the number of the substituents is not less than 2, respective substituents may be the same, or different.

In formulas of the above-mentioned substituents (38)-(46),

u is an integer of 0 to 4,
v is an integer of 1 to 4,
R^{x} is hydroxy, carboxy, cyano, nitro, —NR^{1x}R^{2x}, —CONR^{1x}R^{2x} or —SO₂NR^{1x}R^{2x},
Z^{1x} is —O—, —CO—, —C(OH)R^{3x}—, —C(═N—OR^{3x})—, —S—, —SO—, —SO₂—, —N(COR^{3x})—, —N(CO₂R^{4x})—, —N(SO₂R^{4x})—, —CO—O—, —O—CO—, —CO—NR^{3x}—, —NR^{3x}—CO—, —NR^{3x}—CO₂—, —NR^{3x}—CO—NH—, —NR^{3x}—SO₂— or —NR^{3x}—C(—NH)—NH—, and
Z^{2x} is —O—, —CO—, —C(OH)R^{3x}—, —C(═N—OR^{3x})—, —S—, —SO—, —SO₂—, —NR^{3x}—, —N(COR^{3x})—, —N(CO₂R^{4x})—, —N(SO₂R^{4x})—, —CO—O—, —O—CO—, —CO—NR^{3x}—, —NR^{3x}—CO, —NR^{3x}—CO₂—, —NR^{3x}—CO—NH—, —NR^{3x}—C(═NH)—NH—, —NR^{3x}—SO₂— or —SO₂—NR^{3x}—.

In the above-mentioned formulas, (CH₂)ᵤ and (CH₂)ᵥ are optionally substituted by not less than 1 (preferably 1 to 5, more preferably 1 to 3) substituent selected from the group consisting of, for example, halogen atom, optionally halogenated C₁₋₄ alkyl and hydroxy, and when u or v is not less than 2, —CH₂CH₂— which is a part of (CH₂)ᵤ and (CH₂)ᵥ may be substituted by —CH═CH— or —C≡C—.

In the above-mentioned formulas, R^{1x} and R^{2x} are the same or different and each is a hydrogen atom or a C₁₋₄ alkyl, or R^{1x} and R^{2x} are optionally bonded to each other to form a ring structure with a nitrogen atom. In the above-mentioned formulas, moreover, R^{3x} is a hydrogen atom or a C₁₋₄ alkyl, and R^{4x} is a C₁₋₄ alkyl.

When R^{1x} and R^{2x} are bonded to each other to form ring structure with nitrogen atom, as the nitrogen-containing heterocyclic group, for example, 3 to 8-membered (preferably 5- or 6-membered) saturated or unsaturated (preferably saturated) aliphatic heterocyclic group such as azetidinyl, pyrrolidinyl, piperidinyl, homopiperidinyl, heptamethyleneimino, morpholinyl, thiomorpholinyl, piperazinyl, homopiperazinyl and the like can be mentioned.

The above-mentioned hydrocarbon group having a ring structure such as C₃₋₁₀ cycloalkyl group, C₃₋₁₀ cycloalkenyl group, C₄₋₁₀ cycloalkadienyl group, C₆₋₁₄ aryl group, C₇₋₁₅ aralkyl group, C₈₋₁₃ arylalkenyl group and C₃₋₁₀ cycloalkyl-C₁₋₆ alkyl group and the like, which are exemplarily recited as the “hydrocarbon group”, may have 1 to 3 substituents at substitutable position (s).

As such substituents, for example,

(i) a substituent selected from substituent group U;
(ii) a C₁₋₁₀ alkyl group optionally substituted by 1 to 3 substituents selected from substituent group U;
(iii) a C₂₋₁₀ alkenyl group optionally substituted by 1 to 3 substituents selected from substituent group U;
(in the present specification, the substituents of the above-mentioned (i)-(iii) are sometimes collectively referred to as “substituent group V”) and the like can be mentioned. When the number of the substituents is not less than 2, respective substituents may be the same or different.

In the present specification, unless otherwise specified, as the “heterocyclic group” of the “optionally substituted heterocyclic group” and “heterocyclyl-” in the substituents, an aromatic heterocyclic group and a non-aromatic heterocyclic group can be mentioned.

As the aromatic heterocyclic group, for example, a 4- to 7-membered (preferably 5- or 6-membered) monocyclic aromatic heterocyclic group containing, as a ring-constituting atom besides carbon atoms, 1 to 4 hetero atoms selected from the group consisting of an oxygen atom, a sulfur atom and a nitrogen atom and a 8 to 12-membered fused aromatic heterocyclic group can be mentioned. As the fused aromatic heterocyclic group, for example, a group derived from a fused ring wherein a ring corresponding to such 4- to 7-membered monocyclic aromatic heterocyclic group, and 1 or 2 rings selected from the group consisting of a 5- or 6-membered ring containing 1 or 2 nitrogen atoms, a 5-membered ring containing one sulfur atom, a benzene ring and the like are condensed, and the like can be mentioned.

As preferable examples of the aromatic heterocyclic group,

monocyclic aromatic heterocyclic groups such as furyl (e.g., 2-furyl, 3-furyl), thienyl (e.g., 2-thienyl, 3-thienyl), pyridyl (e.g., 2-pyridyl, 3-pyridyl, 4-pyridyl), pyrimidinyl (e.g., 2-pyrimidinyl, 4-pyrimidinyl, 5-pyrimidinyl, 6-pyrimidinyl), pyridazinyl (e.g., 3-pyridazinyl, 4-pyridazinyl), pyrazinyl (e.g., 2-pyrazinyl), pyrrolyl (e.g., 1-pyrrolyl, 2-pyrrolyl, 3-pyrrolyl), imidazolyl (e.g., 1-imidazolyl, 2-imidazolyl, 4-imidazolyl, 5-imidazolyl), pyrazolyl (e.g., 1-pyrazolyl, 3-pyrazolyl, 4-pyrazolyl), thiazolyl (e.g., 2-thiazolyl, 4-thiazolyl, 5-thiazolyl), isothiazolyl (e.g., 3-isothiazolyl, 4-isothiazolyl, 5-isothiazolyl), oxazolyl (e.g., 2-oxazolyl, 4-oxazolyl, 5-oxazolyl), isoxazolyl (e.g., 3-isoxazolyl, 4-isoxazolyl, 5-isoxazolyl), oxadiazolyl (e.g., 1,2,4-oxadiazol-5-yl, 1,3,4-oxadiazol-2-yl), thiadiazolyl (e.g., 1,3,4-thiadiazol-2-yl), triazolyl (e.g., 1,2,4-triazol-1-yl, 1,2,4-triazol-3-yl, 1,2,3-triazol-1-yl, 1,2,3-triazol-2-yl, 1,2,3-triazol-4-yl), tetrazolyl (e.g., tetrazol-1-yl, tetrazol-5-yl), triazinyl (e.g., 1,2,4-triazol-1-yl, 1,2,4-triazol-3-yl) and the like; fused aromatic heterocyclic groups such as quinolyl (e.g., 2-quinolyl, 3-quinolyl, 4-quinolyl, 6-quinolyl), isoquinolyl (e.g., 3-isoquinolyl), quinazolyl (e.g., 2-quinazolyl, 4-quinazolyl), quinoxalyl (e.g., 2-quinoxalyl, 6-quinoxalyl), benzofuryl (e.g., 2-benzofuryl, 3-benzofuryl), benzothienyl (e.g., 2-benzothienyl, 3-benzothienyl), benzoxazolyl (e.g., 2-benzoxazolyl), benzisoxazolyl (e.g., 7-benzisoxazolyl), benzothiazolyl (e.g., 2-benzothiazolyl), benzimidazolyl (e.g., benzimidazol-1-yl, benzimidazol-2-yl, benzimidazol-5-yl), benzotriazolyl (e.g., 1H-1,2,3-benzotriazol-5-yl), indolyl (e.g., indol-1-yl, indol-2-yl, indol-3-yl, indol-5-yl), indazolyl (e.g., 1H-indazol-3-yl), pyrrolopyrazinyl (e.g., 1H-pyrrolo[2,3-b]pyrazin-2-yl, 1H-pyrrolo[2,3-b]pyrazin-6-yl), imidazopyridinyl (e.g., 1H-imidazo[4,5-b]pyridin-2-yl, 1H-imidazo[4,5-c]pyridin-2-yl, 2H-imidazo[1,2-a]pyridin-3-yl), imidazopyrazinyl (e.g., 1H-imidazo[4,5-b]pyrazin-2-yl), pyrazolopyridinyl (e.g., 1H-pyrazolo[4,3-c]pyridin-3-yl), pyrazolothienyl (e.g., 2H-pyrazolo[3,4-b]thiophen-2-yl), pyrazolotriazinyl (e.g., pyrazolo[5,1-c][1,2,4]triazin-3-yl) and the like; and the like can be mentioned.

As the non-aromatic heterocyclic group, for example, a 4- to 7-membered (preferably 5- or 6-membered) monocyclic non-aromatic heterocyclic group containing, as a ring-constituting atom besides carbon atoms, 1 to 4 hetero atoms selected from the group consisting of an oxygen atom, a sulfur atom and a nitrogen atom and a 8 to 12-membered fused non-aromatic heterocyclic group can be mentioned. As the fused non-aromatic heterocyclic group, for example, a group derived from a fused ring wherein a ring corresponding to such 4- to 7-membered monocyclic non-aromatic heterocyclic group, and 1 or 2 rings selected from the group consisting of a 5- or 6-membered ring containing 1 or 2 nitrogen atoms, a 5-membered ring containing one sulfur atom, a benzene ring and the like are condensed, and the like can be mentioned.

As preferable examples of the non-aromatic heterocyclic group,

monocyclic non-aromatic heterocyclic group such as oxetanyl (e.g., 2-oxetanyl, 3-oxetanyl), pyrrolidinyl (e.g., 1-pyrrolidinyl, 2-pyrrolidinyl), piperidinyl (e.g., piperidino, 2-piperidinyl, 3-piperidinyl, 4-piperidinyl), morpholinyl (e.g., morpholino), thiomorpholinyl (e.g., thiomorpholino), piperazinyl (e.g., 1-piperazinyl, 2-piperazinyl, 3-piperazinyl), hexamethyleneiminyl (e.g., hexamethyleneimine-1-yl), oxazolidinyl (e.g., oxazolidin-2-yl), thiazolidinyl (e.g., thiazolidin-2-yl), imidazolidinyl (e.g., imidazolidin-2-yl, imidazolidin-3-yl), oxazolinyl (e.g., oxazolin-2-yl), thiazolinyl (e.g., thiazolin-2-yl), imidazolinyl (e.g., imidazolin-2-yl, imidazolin-3-yl), dioxolyl (e.g., 1,3-dioxol-4-yl), dioxolanyl (e.g., 1,3-dioxolane-4-yl), dihydrooxadiazolyl (e.g., 4,5-dihydro-1,2,4-oxadiazol-3-yl), 2-thioxo-1,3-oxazolidin-5-yl, pyranyl (e.g., 4-pyranyl), tetrahydropyranyl (e.g., 2-tetrahydropyranyl, 3-tetrahydropyranyl, 4-tetrahydropyranyl), thiopyranyl (e.g., 4-thiopyranyl), tetrahydrothiopyranyl (e.g., 2-tetrahydrothiopyranyl, 3-tetrahydrothiopyranyl, 4-tetrahydrothiopyranyl), 1-oxidetetrahydrothiopyranyl (e.g., 1-oxidetetrahydrothiopyran-4-yl), 1,1-dioxidetetrahydrothiopyranyl (e.g., 1,1-dioxidetetrahydrothiopyran-4-yl), tetrahydrofuryl (e.g., tetrahydrofuran-3-yl, tetrahydrofuran-2-yl), pyrazolidinyl (e.g., pyrazolidin-1-yl, pyrazolidin-3-yl), pyrazolinyl (e.g., pyrazolin-1-yl), tetrahydropyrimidinyl (e.g., tetrahydropyrimidin-1-yl), dihydrotriazolyl (e.g., 2,3-dihydro-1H-1,2,3-triazol-1-yl), tetrahydrotriazolyl (e.g., 2,3,4,5-tetrahydro-1H-1,2,3-triazol-1-yl) and the like; fused non-aromatic heterocyclic group such as dihydroindolyl (e.g., 2,3-dihydro-1H-isoindol-1-yl), dihydroisoindolyl (e.g., 1,3-dihydro-2H-isoindol-2-yl), dihydrobenzofuranyl (e.g., 2,3-dihydro-1-benzofuran-5-yl), dihydrobenzodioxynyl (e.g., 2,3-dihydro-1,4-benzodioxynyl), dihydrobenzodioxepinyl (e.g., 3,4-dihydro-2H-1,5-benzodioxepinyl), tetrahydrobenzofuranyl (e.g., 4,5,6,7-tetrahydro-1-benzofuran-3-yl), chromenyl (e.g., 4H-chromen-2-yl, 2H-chromen-3-yl), dihydroquinolinyl (e.g., 1,2-dihydroquinolin-4-yl), tetrahydroquinolinyl (e.g., 1,2,3,4-tetrahydroquinolin-4-yl), dihydroisoquinolinyl (e.g., 1,2-dihydroisoquinolin-4-yl), tetrahydroisoquinolinyl (e.g., 1,2,3,4-tetrahydroisoquinolin-4-yl), dihydrophthalazinyl (e.g., 1,4-dihydrophthalazin-4-yl) and the like;
and the like can be mentioned.

The “heterocyclic group” of the “optionally substituted heterocyclic group” optionally has 1 to 3 substituents at substitutable positions. As such substituents, for example, substituents selected from above-mentioned Substituent Group V can be mentioned. When the number of the substituents is not less than 2, respective substituents may be the same or different.

In the present specification, unless otherwise specified, as the “heteroaryl group”, the above-mentioned monocyclic aromatic heterocyclic group and fused aromatic heterocyclic group and the like can be mentioned. As the fused aromatic heterocyclic group, a heterocycle wherein the above-mentioned 5- or 6-membered monocyclic aromatic heterocyclic group has been condensed with a benzene ring, or a heterocycle wherein the same or different two heterocycles from the above-mentioned 5- or 6-membered monocyclic aromatic heterocyclic groups have been condensed is preferable.

In the present specification, unless otherwise specified, as the “aliphatic hydrocarbon group” of the “optionally substituted aliphatic hydrocarbon group”, a linear or branched aliphatic hydrocarbon group having 1 to 10 carbon atoms (preferably, 1 to 8 carbon atoms) can be mentioned. As the “aliphatic hydrocarbon group”, for example, a C₁₋₁₀ alkyl group, a C₂₋₁₀ alkenyl group, a C₂₋₁₀ alkynyl group and a C₃₋₁₀ cycloalkyl group can be mentioned (each group is as defined above).

The “aliphatic hydrocarbon group” is optionally substituted by substituents selected from Substituent Group U, particularly, 1 to 3 substituents selected from the group consisting of a halogen atom, hydroxy, C₁₋₄ alkyloxy, C₁₋₄ alkyl-carbonyl, carboxy, C₁₋₄ alkoxy-carbonyl, cyano, carbamoyl, sulfamoyl, nitro, amino, C₁₋₄ alkyl-carbonylamino, C₁₋₄ alkoxy-carbonylamino and C₁₋₄ alkylsulfonylamino.

In the present specification, unless otherwise specified, as the “acyl group”, for example, —COR²¹, —CO—OR²¹, —SO₂R²¹, —SOR²¹, —PO(OR²¹)(OR²²), —CO—NR²³R²⁴, —CO—N(OR²³)R²⁴, —CS—NR²³R²⁴ [wherein R²¹ and R²² are the same or different and each is a hydrogen atom, an optionally substituted hydrocarbon group, or an optionally substituted heterocyclic group. R²³ and R²⁴ are the same or different and each is a hydrogen atom, an optionally substituted hydrocarbon group, or an optionally substituted heterocyclic group, or R²³ and R²⁴ may in combination form, together with the adjacent nitrogen atom, optionally substituted nitrogen-containing heterocycle] and the like can be mentioned. As the “nitrogen-containing heterocycle”, for example, a 3- to 8-membered nitrogen-containing heterocycle containing, as a ring constituent atom besides carbon atoms, at least one nitrogen atom, and further optionally containing 1 or 2 heteroatoms selected from the group consisting of oxygen atom, sulfur atom and nitrogen atom can be mentioned. As preferable examples of the nitrogen-containing heterocycle, 5- or 6-membered cyclic amino optionally containing oxygen atom (e.g., 1-pyrrolidinyl group, piperidinyl, 1-piperazinyl, morpholinyl) can be mentioned. The “nitrogen-containing heterocycle” optionally has 1 to 3 substituents at substitutable position(s). As such substituents, for example, the substituents selected from the above-mentioned Substituent group V can be mentioned. When the number of the substituents is not less than 2, respective substituents may be the same or different.

In the present specification, unless otherwise specified, the “amino group” of the “optionally substituted amino group”, the “carbamoyl group” of the “optionally substituted carbamoyl group”, the “ureido group” of the “optionally substituted ureido group” and the “sulfamoyl group” of the “optionally substituted sulfamoyl group” optionally have 1 or 2 substituents at substitutable position(s). As such substituents, for example, an optionally substituted hydrocarbon group, an optionally substituted heterocyclic group, an acyl group and the like can be mentioned. Of these, 1 or 2 substituents selected from Substituent Group T are preferable. When the number of the substituents is not less than 2, respective substituents may be the same or different.

When the nitrogen atom constituting the above-mentioned amino group, carbamoyl group, ureido group or sulfamoyl group is substituted by two substituents, these substituents may in combination form, together with the adjacent nitrogen atom, a nitrogen-containing heterocycle. As the “nitrogen-containing heterocycle”, for example, a 3 to 8-membered nitrogen-containing heterocycle containing, as a ring-constituting atom besides carbon atoms, at least one nitrogen atom and optionally further containing one or two heteroatoms selected from the group consisting of an oxygen atom, a sulfur atom and a nitrogen atom can be mentioned. As preferable examples of the nitrogen-containing heterocycle, a 5- or 6-membered cyclic amino optionally containing an oxygen atom (e.g., 1-pyrrolidinyl group, piperidinyl, 1-piperazinyl, morpholinyl) can be mentioned. The “nitrogen-containing heterocycle” may have 1 to 3 substituents at substitutable position. As such substituents, for example, substituents selected from the above-mentioned Substituent group V can be mentioned. When the number of the substituents is not less than 2, respective substituents may be the same or different.

In the present specification, unless otherwise specified, as the substituent of the “optionally substituted imino group”, for example, the substituent selected from the above-mentioned substituent group T can be mentioned.

In the present specification, unless otherwise specified, as the “optionally substituted group bonded via a carbon atom”, (1) an optionally substituted hydrocarbon group, (2) an acyl group, (3) an optionally substituted heterocyclic group, which has a bond on a carbon atom, (4) a cyano group and the like can be mentioned.

In the present specification, unless otherwise specified, as the “optionally substituted group bonded via a nitrogen atom”, (1) a nitro group, (2) a group represented by the formula: —NR²⁵R²⁶ [wherein R²⁵ is a hydrogen, an optionally substituted hydrocarbon group, an acyl group, an optionally substituted heterocyclic group, a group represented by the formula: —O—R²⁷ (wherein R²⁷ is a hydrogen atom or an optionally substituted hydrocarbon group, an optionally substituted heterocyclic group, or an acyl group), or formula: —S(O)ₜ—R²⁸ (wherein t is an integer of 0 to 2, R²⁸ is a hydrogen atom, or an optionally substituted hydrocarbon group), R²⁶ is a hydrogen atom, an optionally substituted hydrocarbon group or an acyl group, or R²⁵ and R²⁶ are bonded and may in combination form, together with the adjacent nitrogen atom, an optionally substituted cyclic amino group] can be mentioned.

In the present specification, unless otherwise specified, as the “optionally substituted group bonded via an oxygen atom”, a group represented by the formula: —O—R²⁹ (wherein R²⁹ is a hydrogen atom, or an optionally substituted hydrocarbon group, an optionally substituted heterocyclic group or an acyl group) can be mentioned.

In the present specification, unless otherwise specified, as the “optionally, substituted group bonded via a sulfur atom”, a group represented by the formula: —S(O)ₜ—R³⁰ (wherein t is an integer of 0 to 2, R³⁰ is a hydrogen atom, an optionally substituted hydrocarbon group, or an optionally substituted heterocyclic group) can be mentioned.

In the present specification, unless otherwise specified, as the “nitrogen-containing 7-membered or 8-membered ring”, for example, 7- or 8-membered nitrogen-containing heterocycle containing one nitrogen atom, and further optionally containing 1 to 3 (preferably 1 or 2, more preferably 1) hetero atoms selected from the group consisting of, for example, nitrogen atom, oxygen atom, sulfur atom and the like (e.g., azepin, azepan, azocine, azocane and the like) can be mentioned.

Specific examples of a fused ring of the “nitrogen-containing 7-membered or 8-membered ring” of the “optionally further substituted nitrogen-containing 7-membered or 8-membered ring” for ring A, and a pyrimidine ring, in the above-mentioned formula (I) include

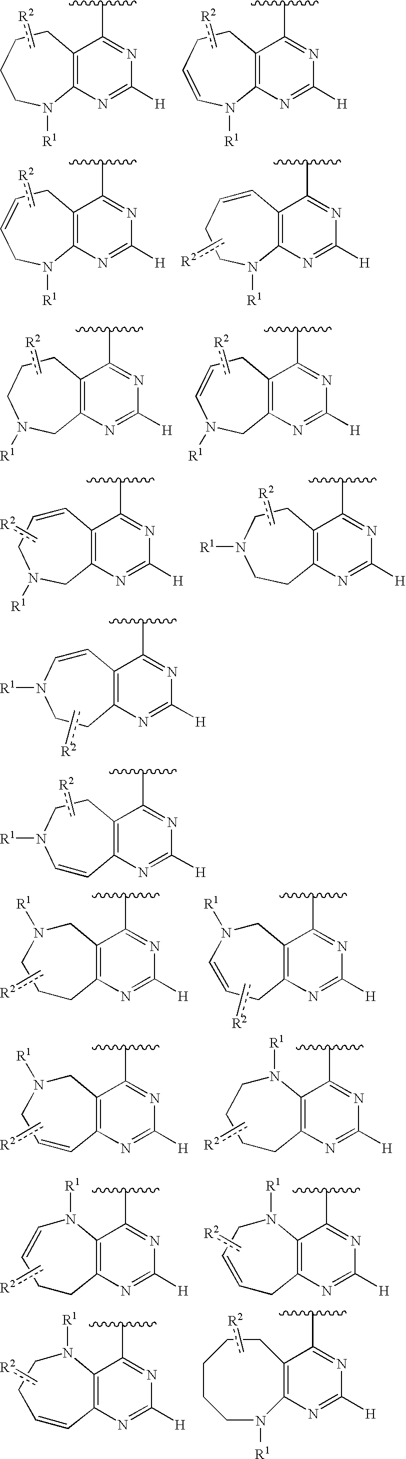

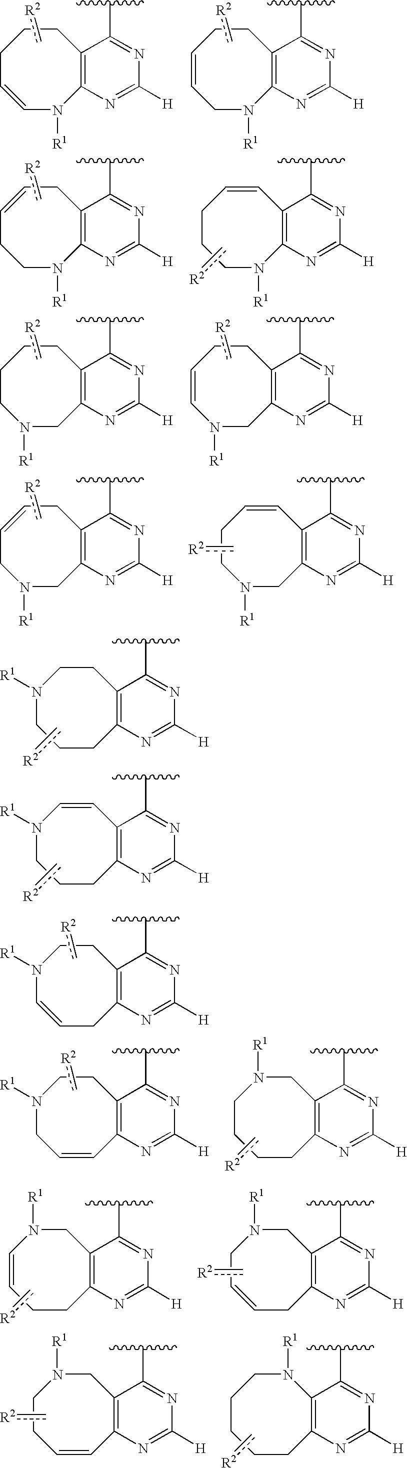

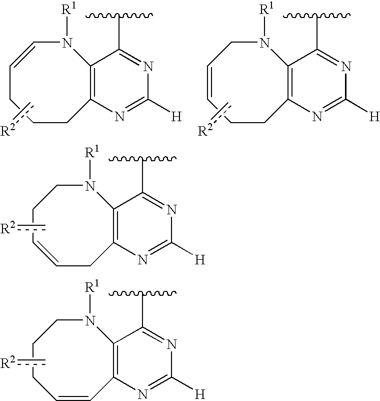

wherein each symbol is as defined above, and the like.

The “nitrogen-containing 7-membered or 8-membered ring” of the “optionally further substituted nitrogen-containing 7-membered or 8-membered ring” for ring A optionally has, besides R¹ group and R² group, 1 to 3 substituents at substitutable position (s). As such substituent, for example, a substituent selected from the above-mentioned Substituent group V can be mentioned. When the number of the substituents is not less than 2, respective substituents may be the same or different.

In the above-mentioned formula (I), as the “aryl group” for B, phenyl is preferable.

In addition, as the “heteroaryl group” for B, the aforementioned “5- or 6-membered monocyclic aromatic heterocyclic group” is preferable, and pyridyl is more preferable.

The “aryl group” or the “heteroaryl group” may be optionally substituted by a group represented by the formula —Y²—W

wherein Y² represents a bond, or formula —X²—(CH₂)ₘ— [wherein X² is a group represented by —O—, —S—, —SO—, —SO₂—, —CH₂— or —CO—NR⁵— (wherein R⁵ is a hydrogen atom, or an optionally substituted C₁₋₆ alkyl group), and m is an integer of 0 to 5]. In the formula, W represents a C₆₋₁₈ aryl group, a heterocyclic group, a C₃₋₇ cycloalkyl group, a carbamoyl group, an ureido group, a C₆₋₁₈ is aryl-carbonyl group or a C₆₋₁₈ aryl-C₁₋₄ alkyl-carbonyl group, each of which is optionally substituted.

As the substituent for the C₆₋₁₈ aryl group, the heterocyclic group, the C₃₋₇ cycloalkyl group, the C₆₋₁₈ aryl-carbonyl group or the C₆₋₁₈ aryl-C₁₋₄ alkyl-carbonyl group, for example, halogen atom, cyano, azido, nitro, nitroso, optionally substituted hydrocarbon group, hydroxy group optionally having optionally substituted hydrocarbon group, thiol group optionally having optionally substituted hydrocarbon group, optionally substituted heterocyclic group, hydroxy group optionally having optionally substituted heterocyclic group, thiol group optionally having optionally substituted heterocyclic group, acyl group, optionally substituted amino group, optionally substituted ureido group, optionally substituted hydrocarbon-sulfonyl group, optionally substituted hydrocarbon-sulfinyl group and the like can be mentioned.

As the substituent for the carbamoyl group or ureido group, for example, an optionally substituted hydrocarbon group, an optionally substituted heterocyclic group, an acyl group and the like can be mentioned.

As the Y², —O— or —OCH₂— is preferable.

As the “C₆₋₁₈ aryl group” for W, phenyl is preferable.

As the “heterocyclic group” for W, the aforementioned 5- or 6-membered monocyclic aromatic heterocyclic group or 5- or 6-membered saturated aliphatic heterocyclic group is preferable, and pyridyl or piperidyl is more preferable.

The “aryl group” or the “heteroaryl group” for B optionally has, besides a group represented by the formula —Y²—W, 1 to 5, same or different substituents at any substitutable position(s). As such substituent, for example, a substituent selected from the above-mentioned substituent group V can be mentioned.

The “C₁₋₄ alkylene” for Y¹ is optionally substituted by 1 to 3 substituents selected from substituent group U.

As the X¹, —NR³— (wherein R³ is as defined above) is preferable, as the R³, a hydrogen atom or a C₁₋₆ alkyl group is preferable.

When [symbol] R² is —R², as the “optionally substituted group bonded via a carbon atom” of the “optionally substituted group bonded via a carbon atom, a nitrogen atom, an oxygen atom or a sulfur atom” for R², an “optionally substituted hydrocarbon group” or an “acyl group” is preferable.

When [symbol] R² is —R², as the “optionally substituted group bonded via a nitrogen atom” of the “optionally substituted group bonded via a carbon atom, a nitrogen atom, an oxygen atom or a sulfur atom” for R², an optionally substituted amino group is preferable.

When [symbol] R² is —R², as the R², an optionally substituted hydrocarbon group, an acyl group, or an optionally substituted amino group is preferable.

As the “optionally substituted group bonded via a carbon atom” of the “optionally substituted group bonded via a carbon atom or a sulfur atom” for R¹, an “optionally substituted hydrocarbon group” is preferable.

As the R¹, a hydrogen atom or a C₁₋₆ alkyl group is preferable. Of these, a hydrogen atom is preferable.

As the “ring structure” when the optionally substituted ring structure was formed by R³ bonded to the carbon atom or heteroatom on the aryl group or heteroaryl group for B, a saturated or unsaturated (preferably saturated) 4- to 8-membered (preferably 5- or 6-membered) nitrogen-containing heterocycle can be mentioned, specifically,

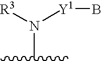

is

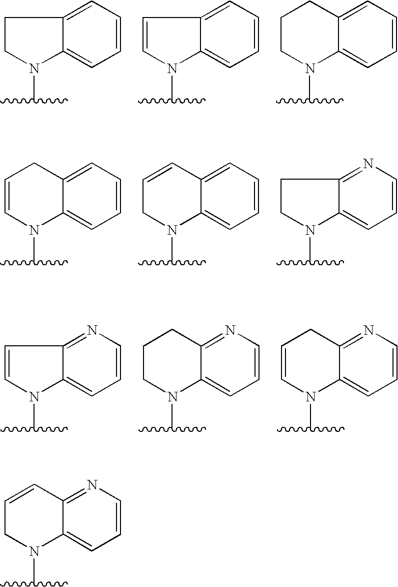

and the like.

The “ring structure” optionally has 1 to 5 (preferably 1 to 3, more preferably 1 or 2), the same or different substituents at any substitutable positions. As such substituents, for example, substituents selected from the above-mentioned Substituent Group V can be mentioned.

R¹ and R² are optionally bonded to each other to form an optionally substituted ring structure. As the “ring structure”, a saturated or unsaturated (preferably saturated) 4- to 8-membered (preferably 5- to 7-membered) heterocycle can be mentioned. When R¹ and R² are bonded to each other to form an optionally substituted ring structure, for example,

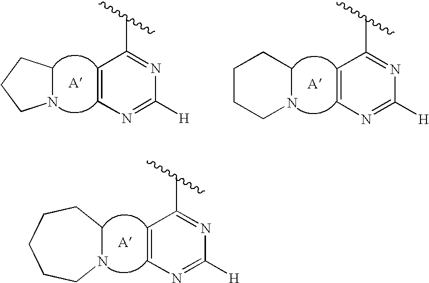

wherein A′ is an optionally further substituted nitrogen-containing 7-membered or 8-membered ring, and other symbols are as defined above, and the like can be mentioned.

R² and R³ are optionally bonded to each other to form an optionally substituted ring structure. As the “ring structure”, a saturated or unsaturated (preferably saturated) 4- to 8-membered (preferably 5- to 7-membered) heterocycle can be mentioned. When R² and R³ are bonded to each other to form an optionally substituted ring structure, for example,

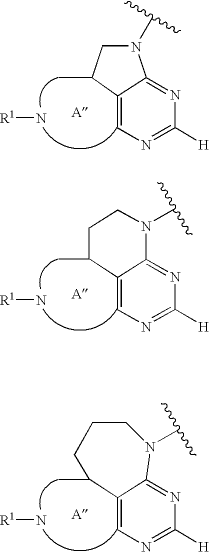

wherein A″ is an optionally further substituted nitrogen-containing 7-membered or 8-membered ring, and other symbols are as defined above, and the like can be mentioned.

The “ring structure” formed by R¹ and R², or R² and R³, which are bonded to each other, optionally has 1 to 5 (preferably 1 to 3, more preferably 1 or 2), same or different substituents selected from the above-mentioned substituent group V at any substitutable position(s).

As compound (I), the following compounds (Ia)-(Ih) and the like are preferably used,

### [Compound (Ia)]

A compound represented by

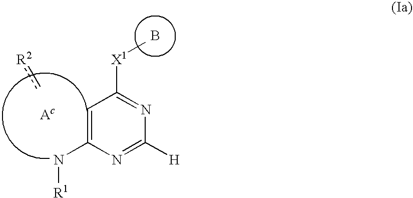

wherein ring A^{a} is an optionally further substituted nitrogen-containing 7-membered or 8-membered ring, and other symbols are as defined above, or a salt thereof.

Specific examples of the fused ring of the “nitrogen-containing 7-membered or 8-membered ring” of the “optionally further substituted nitrogen-containing 7-membered or 8-membered ring” for ring A^{a} and a pyrimidine ring include

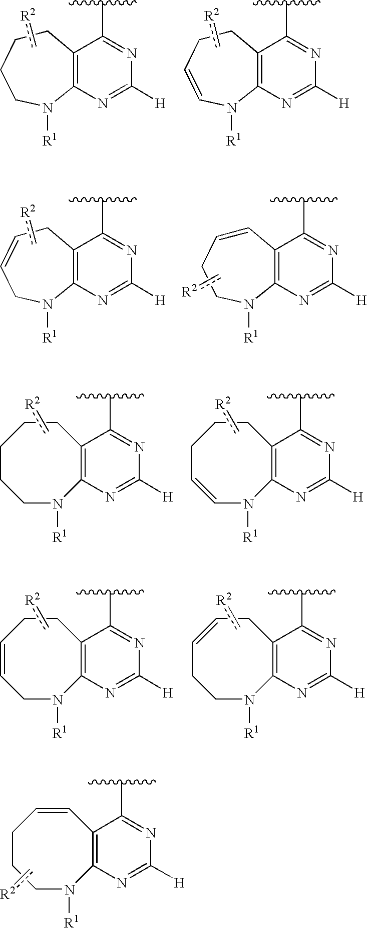

wherein each symbol is as defined above, and the like.

The “nitrogen-containing 7-membered or 8-membered ring” of the “optionally further substituted nitrogen-containing 7-membered or 8-membered ring” for ring A^{a} optionally has, besides R¹ group and R² group, 1 to 3 substituents at substitutable position(s). As such substituent, for example, a substituent selected from the above-mentioned substituent group V can be mentioned. When the number of the substituents is not less than 2, respective substituents may be the same or different.

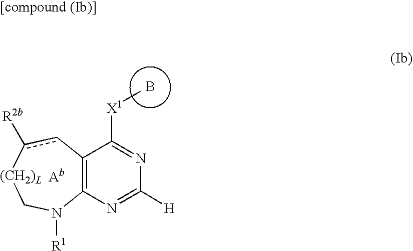

wherein ring A^{b} is an optionally further substituted nitrogen-containing 7-membered or 8-membered ring, L is 1 or 2, formula [symbol] is a single bond or a double bond, R^{2b} is a hydrogen atom, or an optionally substituted group bonded via a carbon atom, a nitrogen atom, an oxygen atom or a sulfur atom, and other symbols are as defined above.

In the above-mentioned formula, as the “optionally substituted group bonded via a carbon atom, a nitrogen atom, an oxygen atom or a sulfur atom” for R^{2b}, those similar to the “optionally substituted group bonded via a carbon atom, a nitrogen atom, an oxygen atom or a sulfur atom” for R² can be used.

As R^{2b},

(i) a C₁₋₆ alkyl group optionally substituted by substituents selected from the group consisting of

(a) hydroxy,

(b) —NH—CO—(CH₂) P—SO₂—C₁₋₆ alkyl (p is an integer of 1 to 6),

(c) —NH—CO—C₁₋₆ alkyl-hydroxy,

(d) —NH—CO—(CH₂)_{p′}—C₁₋₆ alkoxy-C₁₋₆ alkoxy (p′ is an integer of 1-6),

(e) C₁₋₆ alkoxyimino optionally substituted by substituent selected from the group consisting qf (1) hydroxy, (2) C₁₋₆ alkoxy, (3) di-C₁₋₆ alkylamino, (4) C₁₋₆ alkylsulfonyl and (5) 5- to 8-membered heterocyclic group containing, besides carbon atoms, 1 to 3 heteroatoms selected from the group consisting of nitrogen atom, oxygen atom and sulfur atom,

(f) C₁₋₆ alkylamino having cyano,

(g) C₁₋₆ alkylamino having halogen atom,

(h) C₁₋₆ alkylamino having hydroxy,

(i) C₁₋₆ alkylamino having C₁₋₆ alkoxy,

(j) C₁₋₆ alkylamino having C₁₋₆ alkylsulfonyl optionally having hydroxy,

(k) di-C₁₋₆ alkylamino optionally having 1 or 2 substituents selected from the group consisting of (1) hydroxy, (2), cyano, (3) halogen atom and (4) C₁₋₆ alkylsulfonyl,

(l) C₃₋₇ cycloalkylamino optionally having hydroxy,

(m) 5- to 8-membered heterocyclyl-amino containing, besides carbon atoms, 1 to 3 heteroatoms selected from the group consisting of nitrogen atom, oxygen atom and sulfur atom,

(o) 5- to 8-membered cyclic amino optionally having C₁₋₆ alkoxy or C₁₋₆ alkylsulfonyl,

(p) N—C₁₋₆ alkyl-N—C₃₋₇ cycloalkylamino optionally having C₁₋₆ alkylsulfonyl,

(q) cyano,

(r) C₁₋₆ alkylamino having C₁₋₆ alkoxy optionally having hydroxy or C₁₋₆ alkoxy, and

(s) 5- to 8-membered heterocycle substituted by substituents selected from the group consisting of oxo, C₁₋₆ alkylsulfonyl, and C₁₋₆ alkoxy, and containing, besides carbon atoms, 1 to 3 heteroatoms selected from the group consisting of nitrogen atom, oxygen atom and sulfur atom,

(ii) a C₂₋₆ alkenyl group having (a) hydroxy, (b) di-C₁₋₆ alkylamino or (c) C₁₋₆ alkoxy-carbonyl,
(iii) a C₁₋₆ alkoxy-carbonyl group,
(iv) a group represented by —CO—NR^{a}R^{b}

wherein R^{a} is a hydrogen atom or a C₁₋₆ alkyl group, R^{b} is a group represented by
(a) a C₁₋₆ alkyl group optionally substituted by 1 or 2 substituents selected from the group consisting of
   (1) hydroxy,
   (2) amino,
   (3) C₁₋₆ alkylamino having hydroxy,
   (4) C₁₋₆ alkylamino having C₁₋₆ alkoxy,
   (5) cyano,
   (6) amino mono- or di-substituted by C₁₋₆ alkyl optionally having hydroxy,
   (7) C₁₋₆ alkyl-carbonylamino,
   (8) C₁₋₆ alkoxy,
   (9) C₁₋₆ alkoxy having hydroxy,
   (10) C₁₋₆ alkoxy having C₁₋₆ alkoxy,
   (11) C₁₋₆ alkoxy having hydroxy and C₁₋₆ alkoxy,
   (12) C₁₋₆ alkoxy having C₁₋₆ alkylsulfonyl,
   (13) C₁₋₆ alkoxy having cyano,
   (14) C₁₋₆ alkoxy-carbonyl,
   (15) C₁₋₆ alkylsulfonyl optionally having hydroxy or C₁₋₆ alkoxy,
   (16) 5- to 8-membered heterocycle containing, besides carbon atoms, 1 to 3 heteroatoms selected from the group consisting of nitrogen atom, oxygen atom and sulfur atom-sulfonyl,
   (17) C₆₋₁₈ arylsulfonyl,
   (18) 5- to 8-membered heterocycle optionally having 1 to 2 substituents selected from the group consisting of hydroxy, C₁₋₆ alkyl, C₆₋₁₈ aryl, and C₆₋₁₈ aryl-C₁₋₆ alkyl, and containing 1 to 3 heteroatoms selected from the group consisting of nitrogen atom, oxygen atom and sulfur atom, besides carbon atoms,
   (19) C₃₋₇ cycloalkyl optionally having hydroxy, and
   (20) C₆₋₁₈ aryl optionally having 1 to 2 halogens,
(b) a C₂₋₆ alkenyl group,
(c) a C₃₋₇ cycloalkyl group optionally having hydroxy,
(d) a C₁₋₆ alkoxy group, or
(e) a 5- to 8-membered heterocyclic group optionally having 1 or 2 substituents selected from the group consisting of hydroxy, C₁₋₆ alkyl, C₆₋₁₈ aryl, and C₆₋₁₈ aryl-C₁₋₆ alkyl, and containing 1 to 3 heteroatoms selected from the group consisting of nitrogen atom, oxygen atom and sulfur atom, besides carbon atoms,
   (v) a 5- to 8-membered cyclic amino-carbonyl group optionally having substituents selected from the group consisting of

(a) hydroxy,

(b) C₁₋₆ alkylsulfonyl, and

(c) C₁₋₆ alkyl optionally having C₁₋₆ alkylsulfonyl,

(vi) a carboxy group,
(vii) an amino group optionally substituted by C₁₋₆ alkoxy-carbonyl optionally having C₁₋₆ alkylsulfonyl, or
(viii) a 5- to 8-membered heterocyclic group optionally having C₁₋₆ alkyl, and containing 1 to 3 heteroatoms selected from the group consisting of nitrogen atom, oxygen atom and sulfur atom, besides carbon atoms is preferable.

Specific examples of the fused ring of the “nitrogen-containing 7-membered or 8-membered ring” of the “optionally further substituted nitrogen-containing 7-membered or 8-membered ring” for ring A^{b} and a pyrimidine ring include the following structures:

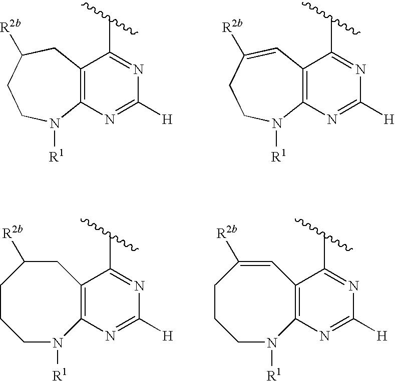

wherein each symbol is as defined above.

The “nitrogen-containing 7-membered or 8-membered ring” of the “optionally further substituted nitrogen-containing 7-membered or 8-membered ring” for ring A^{b} optionally has, besides R¹ group and R^{2b} group, 1 to 3 substituents at substitutable position(s). As such substituent, for example, a substituent selected from the above-mentioned substituent group V can be mentioned. When the number of substituents is not less than 2, respective substituents may be the same or different.

### [Compound (Ic)]

A compound represented by

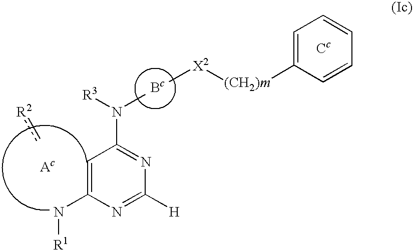

wherein ring A^{c} is an optionally further substituted nitrogen-containing 7-membered or 8-membered ring, ring B^{c} is an optionally further substituted phenyl group or an optionally further substituted pyridyl group, ring C^{c} is an optionally substituted phenyl group, X² is a group represented by —O—, —S—, —SO—, —SO₂—, —CH₂— or —CO—NR⁵— (wherein R⁵ is a hydrogen atom, or an optionally substituted C₁₋₆ alkyl group), m is an integer of 0 to 5, and other symbols are as defined above, R³ may be bonded to the carbon atom on ring B^{c} to form an optionally substituted ring structure, or a salt thereof.

In the above-mentioned formula, as the “optionally further substituted nitrogen-containing 7-membered or 8-membered ring” for ring A^{c}, those similar to the above-mentioned “optionally further substituted nitrogen-containing 7-membered or 8-membered ring” for ring A^{a} can be used.

As the substituent for the “optionally substituted phenyl group” for ring C^{c}, for example, 1 to 5, same or different substituents selected from the above-mentioned substituent group V can be used.

As the substituent for the “optionally further substituted phenyl group” or the “optionally further substituted pyridyl group” for ring B^{c}, for example, 1 to 4, same or different substituents selected from the above-mentioned substituent group V can be used.

As the “optionally substituted ring structure” formed by R³ bonded to the carbon atom on ring B^{c}, those similar to the “optionally substituted ring structure” formed by R³ bonded to the carbon atom on ring B can be used.

### [Compound (Id)]

A compound represented by

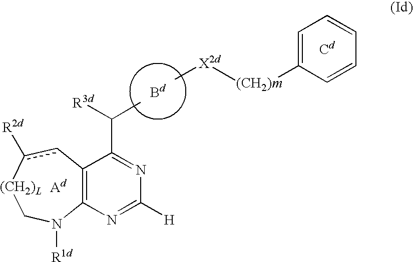

wherein ring A^{d} is an optionally further substituted nitrogen-containing 7-membered or 8-membered ring, L is 1 or 2,
formula [symbol] is a single bond or a double bond, ring B^{d} is an optionally further substituted phenyl group or an optionally further substituted pyridyl group,
ring C^{d} is an optionally substituted phenyl group,
X^{2d} is a group represented by —O—, —S—, —SO—, —SO₂, —CH₂— or —CO—NR^{5d}— (wherein R^{5d} is a hydrogen atom, or an optionally substituted C₁₋₆ alkyl group),
m is an integer of 0 to 5,
R^{1d} is a hydrogen atom, or an optionally substituted group bonded via a carbon atom or a sulfur atom,
R^{2d} is a hydrogen atom, or an optionally substituted group bonded via a carbon atom, a nitrogen atom, an oxygen atom or a sulfur atom,
R^{3d} represents a hydrogen atom or an optionally substituted aliphatic hydrocarbon group, or R^{3d} is optionally bonded to the carbon atom on ring B^{d} to form an optionally substituted ring structure, or a salt thereof.

Specific examples of the “nitrogen-containing 7-membered or 8-membered ring” of the “optionally further substituted nitrogen-containing 7-membered or 8-membered ring” for ring A^{d} include 7-membered or 8-membered ring shown by the following structures:

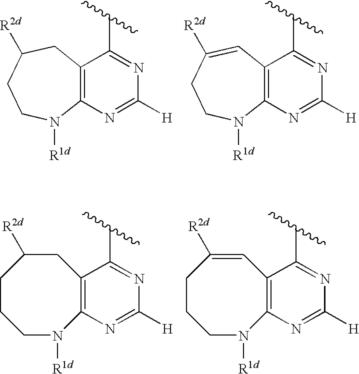

wherein each symbol is as defined above.

The “nitrogen-containing 7-membered or 8-membered ring” of the “optionally further substituted nitrogen-containing 7-membered or 8-membered ring” for ring A^{d} optionally has, besides R^{1d} group and R^{2d} group, 1 to 3 substituents at substitutable position (s). As such substituent, for example, a substituent selected from the above-mentioned substituent group V can be mentioned. When the number of the substituents is not less than 2, respective substituents may be the same or different.

As the further substituents for the “optionally further substituted phenyl group” or the “optionally further substituted pyridyl group” for ring B^{d}, those similar to ring B^{c} can be used, and C₁₋₆ alkyl group or halogen atom is preferable.

As the further substituents for ring C^{d}, those similar to ring C^{c} can be used, and optionally halogenated C₁₋₆ alkyl, optionally halogenated C₁₋₆ alkoxy, C₁₋₆ alkyl-carbamoyl or halogen atom is preferable.

As X^{2d}, those similar to X² can be used. When X^{2d} is —CONR^{5d}—, as the substituent for the “optionally substituted C₁₋₆ alkyl group” represented by R^{5d}, those similar to R⁵ can be used.

As “—X^{2d}—(CH₂)ₘ—”, —O— or —O—CH₂— is preferable.

As the “optionally substituted group bonded via a carbon atom, or a sulfur atom” for R^{1d}, those similar to the “optionally substituted group bonded via a carbon atom or a sulfur atom” for R¹ can be used.

As the R^{1d}, a hydrogen atom or a C₁₋₆ alkyl group is preferable.

As the “optionally substituted group bonded via a carbon atom, a nitrogen atom, an oxygen atom or a sulfur atom” for R^{2d}, those similar to the “optionally substituted group bonded via a carbon atom, a nitrogen atom, an oxygen atom or a sulfur atom” for R² can be used.

As R^{2d},

(i) a C₁₋₆ alkyl group optionally substituted by substituents selected from the group consisting of

(a) hydroxy,

(b) —NH—CO—(CH₂)ₚ—SO₂—C₁₋₆ alkyl (p is an integer of 1 to 6),

(c) —NH—CO—C₁₋₆ alkyl-hydroxy,

(d) —NH—CO—(CH₂)_{p′}—C₁₋₆ alkoxy-C₁₋₆ alkoxy (p′ is an integer of 1 to 6),

(e) C₁₋₆ alkoxyimino optionally substituted substituent selected from the group consisting of (1) hydroxy, (2) C₁₋₆ alkoxy, (3) di-C₁₋₆ alkylamino, (4) C₁₋₆ alkylsulfonyl and (5) 5- to 8-membered heterocyclic group containing, besides carbon atoms, 1 to 3 heteroatoms selected from the group consisting of nitrogen atom, oxygen atom and sulfur atom,

(f) C₁₋₆ alkylamino having cyano,

(g) C₁₋₆ alkylamino having halogen atom,

(h) C₁₋₆ alkylamino having hydroxy,

(i) C₁₋₆ alkylamino having C₁₋₆ alkoxy,

(j) C₁₋₆ alkylamino having C₁₋₆ alkylsulfonyl optionally having hydroxy,

(k) di-C₁₋₆ alkylamino optionally having 1 or 2 substituents selected from the group consisting of (1) hydroxy, (2) cyano, (3) halogen atom and (4) C₁₋₆ alkylsulfonyl,

(l) C₃₋₇ cycloalkylamino optionally having hydroxy,

(m) 5- to 8-membered heterocyclyl-amino containing, besides carbon atoms, 1 to 3 heteroatoms selected from the group consisting of nitrogen atom, oxygen atom and sulfur atom,

(o) 5- to 8-membered cyclic amino optionally having C₁₋₆ alkoxy or C₁₋₆ alkylsulfonyl,

(p) N—C₁₋₆ alkyl-N—C₃₋₇ cycloalkylamino optionally having C₁₋₆ alkylsulfonyl,

(q) cyano,

(r) C₁₋₆ alkylamino having C₁₋₆ alkoxy optionally having hydroxy or C₁₋₆ alkoxy, and

(s) 5- to 8-membered heterocycle optionally substituted by substituents selected from the group consisting of oxo, C₁₋₆ alkylsulfonyl, and C₁₋₆ alkoxy, and containing 1 to 3 heteroatoms selected from the group consisting of nitrogen atom, oxygen atom and sulfur atom besides carbon atoms,

(ii) a C₂₋₆ alkenyl group having (a) hydroxy, (b) di-C₁₋₆ alkylamino or (c) C₁₋₆ alkoxy-carbonyl,
(iii) a C₁₋₆ alkoxy-carbonyl group,
(iv) a group represented by —CO—NR^{a}R^{b}

wherein R^{a} is a hydrogen atom or a C₁₋₆ alkyl group,
R^{b} is a group represented by
(a) a C₁₋₆ alkyl group optionally substituted by 1 or 2 substituents selected from the group consisting of
   (1) hydroxy,
   (2) amino,
   (3) C₁₋₆ alkylamino having hydroxy,
   (4) C₁₋₆ alkylamino having C₁₋₆ alkoxy,
   (5) cyano,
   (6) amino mono- or di-substituted by C₁₋₆ alkyl optionally having hydroxy,
   (7) C₁₋₆ alkyl-carbonylamino,
   (8) C₁₋₆ alkoxy,
   (9) C₁₋₆ alkoxy having hydroxy,
   (10) C₁₋₆ alkoxy having C₁₋₆ alkoxy,
   (11) C₁₋₆ alkoxy having hydroxy and C₁₋₆ alkoxy,
   (12) C₁₋₆ alkoxy having C₁₋₆ alkylsulfonyl,
   (13) C₁₋₆ alkoxy having cyano,
   (14) C₁₋₆ alkoxy-carbonyl,
   (15) C₁₋₆ alkylsulfonyl optionally having hydroxy or C₁₋₆ alkoxy,
   (16) 5- to 8-membered heterocycle containing, besides carbon atoms, 1 to 3 heteroatoms selected from the group consisting of nitrogen atom, oxygen atom and sulfur atom-sulfonyl,
   (17) C₆₋₁₈ arylsulfonyl,
   (18) 5- to 8-membered heterocycle optionally having 1 or 2 substituents selected from the group consisting of hydroxy, C₁₋₆ alkyl, C₆₋₁₈ is aryl, and C₆₋₁₈ aryl-C₁₋₆ alkyl, and containing, besides carbon atoms, 1 to 3 heteroatoms selected from the group consisting of a nitrogen atom, an oxygen atom and a sulfur atom,
   (19) C₃₋₇ cycloalkyl optionally having hydroxy, and
   (20) C₆₋₁₈ aryl optionally having 1 or 2 halogens,
(b) a C₂₋₆ alkenyl group,
(c) a C₃₋₇ cycloalkyl group optionally having hydroxy,
(d) a C₁₋₆ alkoxy group, or
(e) a 5- to 8-membered heterocyclic group optionally having 1 or 2 substituents selected from the group consisting of hydroxy, C₁₋₆ alkyl, C₆₋₁₈ aryl, and C₆₋₁₈ aryl-C₁₋₆ alkyl, and containing 1 to 3 heteroatoms selected from the group consisting of nitrogen atom, oxygen atom and sulfur atom besides carbon atoms,
   (v) a 5- to 8-membered cyclic amino-carbonyl group optionally having substituents selected from the group consisting of

(a) hydroxy,

(b) C₁₋₆ alkylsulfonyl, and

(c) C₁₋₆ alkyl optionally having C₁₋₆ alkylsulfonyl,

(vi) a carboxy group,
(vii) an amino group optionally substituted by C₁₋₆ alkoxy-carbonyl optionally having C₁₋₆ alkylsulfonyl, or
(viii) a 5- to 8-membered heterocyclic group optionally having C₁₋₆ alkyl, and containing, besides carbon atoms, 1 to 3 heteroatoms selected from the group consisting of nitrogen atom, oxygen atom and sulfur atom is preferable.

As the substituent for the “optionally substituted aliphatic hydrocarbon group” for R^{3d}, those similar to R³ can be used.

As the R^{3d}, a hydrogen atom is preferable.

As the “optionally substituted ring structure” formed by R^{3d} bonded to the carbon atom on ring B^{d}, those similar to the “optionally substituted ring structure” formed by R³ bonded to the carbon atom on ring B can be used.

As the preferable embodiment for compound (Id), compound (Id)

wherein
ring A^{d} is a nitrogen-containing 7-membered or 8-membered ring (preferably 7-membered ring) without a substituent other than R^{1d} and R^{2d},
L is 1 or 2 (preferably 1),
R^{1d} is a hydrogen atom or a C₁₋₆ alkyl group,

### R^{2d} is

(i) a C₁₋₆ alkyl group optionally substituted by substituents selected from the group consisting of

(a) hydroxy,

(b) —NH—CO—(CH₂)ₚ—SO₂—C₁₋₆ alkyl (p is an integer of 1 to 6),

(c) —NH—CO—C₁₋₆ alkyl-hydroxy,

(d) —NH—CO—(CH₂)ₚ′—C₁₋₆ alkoxy-C₁₋₆ alkoxy (p′ is an integer of 1 to 6),

(e) C₁₋₆ alkoxyimino optionally substituted by

substituents selected from the group consisting of (1) hydroxy, (2) C₁₋₆ alkoxy, (3) di-C₁₋₆ alkylamino, (4) C₁₋₆ alkylsulfonyl and (5) 5- to 8-membered heterocyclic group containing 1 to 3 heteroatoms selected from the group consisting of nitrogen atom, oxygen atom and sulfur atom besides carbon atoms,

(f) C₁₋₆ alkylamino having cyano,

(g) C₁₋₆ alkylamino having halogen atom,

(h) C₁₋₆ alkylamino having hydroxy,

(i) C₁₋₆ alkylamino having C₁₋₆ alkoxy,

(j) C₁₋₆ alkylamino having C₁₋₆ alkylsulfonyl optionally having hydroxy,

(k) di-C₁₋₆ alkylamino optionally having 1 or 2 substituents selected from the group consisting of (1) hydroxy, (2) cyano, (3) halogen atom and (4) C₁₋₆ alkylsulfonyl,

(l) C₃₋₇ cycloalkylamino optionally having hydroxy,

(m) 5- to 8-membered heterocyclyl-amino containing, besides carbon atoms, 1 to 3 heteroatoms selected from the group consisting of nitrogen atom, oxygen atom and sulfur atom,

(o) 5- to 8-membered cyclic amino optionally having C₁₋₆ alkoxy or C₁₋₆ alkylsulfonyl,

(p) N—C₁₋₆ alkyl-N—C₃₋₇ cycloalkylamino optionally having C₁₋₆ alkylsulfonyl.

(q) cyano,

(r) C₁₋₆ alkylamino having C₁₋₆ alkoxy optionally having hydroxy or C₁₋₆ alkoxy, and

(s) 5- to 8-membered heterocycle substituted by substituents selected from the group consisting of oxo, C₁₋₆ alkylsulfonyl, and C₁₋₆ alkoxy, containing, besides carbon atoms, 1 to 3 heteroatoms selected from the group consisting of nitrogen atom, oxygen atom and sulfur atom,

(ii) a C₂₋₆ alkenyl group having (a) hydroxy, (b) di-C₁₋₆ alkylamino or (c) C₁₋₆ alkoxy-carbonyl,
(iii) a C₁₋₆ alkoxy-carbonyl group,
(iv) a group represented by —CO—NR^{a}R^{b}

wherein R^{a} is a hydrogen atom or a C₁₋₆ alkyl group,
R^{b} is a group represented by
   (a) a C₁₋₆ alkyl group optionally substituted by 1 or 2 substituents selected from the group consisting of
      (1) hydroxy,
      (2) amino,
      (3) C₁₋₆ alkylamino having hydroxy,
      (4) C₁₋₆ alkylamino having C₁₋₆ alkoxy,
      (5) cyano,
      (6) amino mono- or di-substituted by C₁₋₆ alkyl optionally having hydroxy,
      (7) C₁₋₆ alkyl-carbonylamino,
      (8) C₁₋₆ alkoxy,
      (9) C₁₋₆ alkoxy having hydroxy,
      (10) C₁₋₆ alkoxy having C₁₋₆ alkoxy,
      (11) C₁₋₆ alkoxy having hydroxy and C₁₋₆ alkoxy,
      (12) C₁₋₆ alkoxy having C₁₋₆ alkylsulfonyl,
      (13) C₁₋₆ alkoxy having cyano,
      (14) C₁₋₆ alkoxy-carbonyl,
      (15) C₁₋₆ alkylsulfonyl optionally having hydroxy or C₁₋₆ alkoxy,
      (16) 5- to 8-membered heterocyclyl-sulfonyl containing, besides carbon atoms, 1 to 3 heteroatoms selected from the group consisting of nitrogen atom, oxygen atom and sulfur atom,
      (17) C₆₋₁₈ arylsulfonyl,
      (18) 5- to 8-membered heterocycle optionally having 1 or 2 substituents selected from the group consisting of hydroxy, C₁₋₆ alkyl, C₆₋₁₈ aryl, and C₆₋₁₈ aryl-C₁₋₆ alkyl, and containing, besides carbon atoms, 1 to 3 heteroatoms selected from the group consisting of nitrogen atom, oxygen atom and sulfur atom,
      (19) C₃₋₇ cycloalkyl optionally having hydroxy, and
      (20) C₆₋₁₈ aryl optionally having 1 to 2 halogens,
   (b) a C₂₋₆ alkenyl group,
   (c) a C₃₋₇ cycloalkyl group optionally having hydroxy,
   (d) a C₁₋₆ alkoxy group, or
   (e) a 5- to 8-membered heterocyclic group optionally having 1 or 2 substituents selected from the group consisting of hydroxy, C₁₋₆ alkyl, C₆₋₁₈ aryl, and C₆₋₁₈ aryl-C₁₋₆ alkyl, and containing, besides carbon atoms, 1 to 3 heteroatoms selected from the group consisting of nitrogen atom, oxygen atom and sulfur atom,
      (v) a 5- to 8-membered cyclic amino-carbonyl group optionally having substituents selected from the group consisting of

(a) hydroxy,

(b) C₁₋₆ alkylsulfonyl, and

(c) C₁₋₆ alkyl optionally having C₁₋₆ alkylsulfonyl,

(vi) a carboxy group,
(vii) an amino group optionally substituted by C₁₋₆ alkoxy-carbonyl optionally having C₁₋₆ alkylsulfonyl, or
(viii) a 5- to 8-membered heterocyclic group optionally having C₁₋₆ alkyl, and containing, besides carbon atoms, 1 to 3 heteroatoms selected from the group consisting of nitrogen atom, oxygen atom and sulfur atom,
R^{3d} is a hydrogen atom,
ring B^{d} is a phenyl group optionally further substituted by a C₁₋₆ alkyl group or a halogen atom, or a pyridyl group optionally further substituted by a C₁₋₆ alkyl group or a halogen atom (preferably, a phenyl group optionally further substituted by a C₁₋₆ alkyl group or a halogen atom),
ring C^{d} is a phenyl group optionally substituted by substituents selected from the group consisting of
(i) optionally halogenated C₁₋₆ alkyl,
(ii) C₁₋₆ alkoxy optionally having halogen atom or C₃₋₇ cycloalkyl,
(iii) C₁₋₆ alkyl-carbamoyl optionally having hydroxy,
(iv) halogen atom,
(v) cyano,
(vi) C₁₋₆ alkylthio optionally having halogen atom,
(vii) C₁₋₆ alkylsulfinyl optionally having halogen atom, and
(viii) C₁₋₆ alkylsulfonyl optionally having halogen atom or C₃₋₇ cycloalkyl,
X^{2d} is a group represented by —O— or —S—,
m is 0 or 1, can be mentioned.

### [Compound (Ie)]

A compound represented by

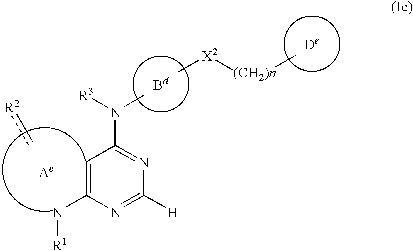

wherein ring A^{e} is an optionally further substituted nitrogen-containing 7-membered or 8-membered ring, ring B^{e} is an optionally further substituted phenyl group or an optionally further substituted pyridyl group, ring D^{e} is an optionally substituted aromatic heterocyclic group, X² is a group represented by —O—, —S—, —SO—, —SO₂—, —CH₂— or —CO—NR⁵— (wherein R⁵ is a hydrogen atom, or an optionally substituted C₁₋₆ alkyl group), n is an integer of 0 to 5, other symbols are as defined above, and R³ may be bonded to the carbon atom on ring B^{e} to form optionally substituted ring structure, or a salt thereof.

In the above-mentioned formula, as the “optionally further substituted nitrogen-containing 7-membered or 8-membered ring” for ring A^{e}, those similar to the above-mentioned “optionally further substituted nitrogen-containing 7-membered or 8-membered ring” for ring A^{a} can be used.

As the “aromatic heterocyclic group” of the “optionally substituted aromatic heterocyclic group” represented by ring D^{e}, those similar to the aforementioned “heteroaryl” can be used. Of those, a 5- or 6-membered monocyclic aromatic heterocyclic group is preferable as ring D^{e}.

As the substituent of the “optionally substituted aromatic heterocyclic group” represented by ring D^{e}, 1 to 5, same or different substituents selected from the above-mentioned substituent group V are used.

As the substituent of the “optionally further substituted phenyl group” or “optionally further substituted pyridyl group” represented by ring B % 1 to 4, same or different substituents selected from the above-mentioned substituent group V are used.

As the ring B^{e}, an “optionally further substituted phenyl group” is preferable.

As the “optionally substituted ring structure” formed by R³ bonded to the carbon atom on ring B^{e}, those similar to the “optionally substituted ring structure” formed by R³ bonded to the carbon atom on ring B can be used.

### [Compound (If)]

A compound represented by

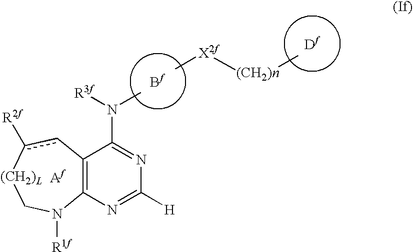

wherein ring A^{f} is an optionally further substituted nitrogen-containing 7-membered or 8-membered ring,

### L is 1 or 2,

formula [symbol] is a single bond or a double bond,
ring B^{f} is an optionally further substituted phenyl group or an optionally further substituted pyridyl group,
ring D^{f} is an optionally substituted aromatic heterocyclic group,
X^{2f} is a group represented by —O—, —S—, —SO—, —SO₂, —CH₂— or —CO—NR^{5f}— (wherein R^{5f} is a hydrogen atom, or an optionally substituted C₁₋₆ alkyl group),
n is an integer of 0 to 5,
R^{1f} is a hydrogen atom, or an optionally substituted group bonded via a carbon atom or a sulfur atom,
R^{2f} is a hydrogen atom, or an optionally substituted group bonded via a carbon atom, a nitrogen atom, an oxygen atom or a sulfur atom, and
R^{3f} is a hydrogen atom, or an optionally substituted aliphatic hydrocarbon group, or R^{3f} is optionally bonded to the carbon atom on ring B^{f} to form an optionally substituted ring structure, or a salt thereof.

Specific examples the “nitrogen-containing 7-membered or 8-membered ring” of the “optionally further substituted nitrogen-containing 7-membered or 8-membered ring” for ring A^{f} include 7-membered or 8-membered ring shown by the following structures:

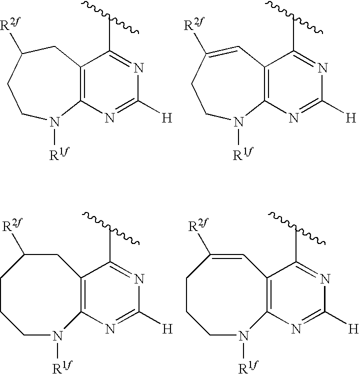

wherein each symbol is as defined above.

The “nitrogen-containing 7-membered or 8-membered ring” of the “optionally further substituted nitrogen-containing 7-membered or 8-membered ring” for ring A^{f} optionally has, besides R^{1f} group and R^{2f} group, 1 to 3 substituents at substitutable position (s). As such substituent, for example, a substituent selected from the above-mentioned substituent group V can be mentioned. When the number of the substituents is not less than 2, respective substituents may be the same or different.

As the “aromatic heterocycle” of the “optionally substituted aromatic heterocycle” for ring D^{f}, those similar to ring D^{e} can be used. Of those, a 5- or 6-membered monocyclic aromatic heterocyclic group is preferable, and a pyridine ring is more preferable.

As the substituent of the “optionally substituted aromatic heterocycle” represented by ring D^{f}, those similar to the above-mentioned substituent group V can be used. Of those, a C₁₋₆ alkyl group is preferable.

As the further substituent of the “optionally further substituted phenyl group” or “optionally further substituted pyridyl group” represented by ring B^{f}, those similar to ring B^{e} can be used. Of those, a C₁₋₆ alkyl group or a halogen atom is preferable.

As ring B^{e}, a phenyl group optionally further substituted by C₁₋₆ alkyl group or halogen atom is preferable.

As X^{2f}, those similar to X² can be used. When X^{2f} is —CONR^{5f}—, as the substituent of the “optionally substituted C₁₋₆ alkyl group” represented by R^{5f}, those similar to R⁵ can be used.

As the “—X^{2f}—(CH₂)ₙ—”, —O— or —O—CH₂— is preferable.

As the “optionally substituted group bonded via a carbon atom or a sulfur atom” represented by R^{1f}, those similar to the “optionally substituted group bonded via a carbon atom or a sulfur atom” represented by R¹ can be used.

As the R^{1f}, a hydrogen atom or a C₁₋₆ alkyl group is preferable.

As the “optionally substituted group bonded via a carbon atom, a nitrogen atom, an oxygen atom or a sulfur atom” represented by R^{2f}, those similar to the “optionally substituted group bonded via a carbon atom, a nitrogen atom, an oxygen atom or a sulfur atom” represented by R² can be used.

As R^{2f},

(i) a C₁₋₆ alkyl group optionally having hydroxy,
(ii) a C₁₋₆ alkoxy-carbonyl group,
(iii) a group represented by —CO—NR^{c}R^{d}

wherein R^{c} is a hydrogen atom,
R^{d} is a group represented by
   (a) a C₁₋₆ alkyl group substituted by substituents selected from the group consisting of (1) C₁₋₆ alkoxy, (2) C₁₋₆ alkoxy optionally having substituents selected from the group consisting of (1′) hydroxy, (2′) cyano and (3′) C₁₋₆ alkoxy, and (3) C₁₋₆ alkylsulfonyl optionally having hydroxy,
   (b) a C₃₋₇ cycloalkyl group, or
   (c) a C₁₋₆ alkoxy group optionally substituted by C₁₋₆ alkylsulfonyl,
      (iv) a 5- to 8-membered cyclic amino-carbonyl group optionally substituted by substituents selected from the group consisting of

(a) a hydroxy, and

(b) a C₁₋₆ alkyl optionally having hydroxy, or

(v) a carboxy group
is preferable.

As the substituent of the “optionally substituted aliphatic hydrocarbon group” represented by ring R^{3f}, those similar to R³ can be used.

As the R^{3f}, a hydrogen atom is preferable.

As the “optionally substituted ring structure” formed by R^{3f} bonded to the carbon atom on ring B^{f}, those similar to the “optionally substituted ring structure” formed by R³ bonded to the carbon atom on ring B can be used.

A preferable embodiment of compound (If) is compound (If) wherein

ring A^{f} is a nitrogen-containing 7-membered or 8-membered ring (preferably 7-membered ring) without a substituent other than R^{1d} and R^{2d},
L is 1 or 2 (preferably 1),
R^{1f} is a hydrogen atom or a C₁₋₆ alkyl group,

### R^{2f} is

(i) a C₁₋₆ alkyl group optionally having hydroxy,
(ii) a C₁₋₆ alkoxy-carbonyl group,
(iii) a group represented by —CO—NR^{c}R^{d}

wherein R^{c} is a hydrogen atom,
R^{d} is
   (a) a C₁₋₆ alkyl group optionally substituted by substituents selected from the group consisting of (1) C₁₋₆ alkoxy, (2) C₁₋₆ alkoxy optionally having substituents selected from the group consisting of (1′) hydroxy, (2′) cyano and (3′) C₁₋₆ alkoxy, and (3) C₁₋₆ alkylsulfonyl optionally having hydroxy,
(b) a C₃₋₇ cycloalkyl group, or
(c) a C₁₋₆ alkoxy group optionally substituted C₁₋₆ alkylsulfonyl,
   (iv) a 5- to 8-membered cyclic amino-carbonyl group optionally substituted by substituents selected from the group consisting of

(a) hydroxy, and

(b) C₁₋₆ alkyl optionally having hydroxy, or

(v) a carboxy group,
R^{3f} is a hydrogen atom,
ring B^{f} is a phenyl group optionally further substituted by a C₁₋₆ alkyl group,
X^{f} is a group represented by —O—,
n is 0, and
ring D^{f} is a 5- or 6-membered monocyclic aromatic heterocycle optionally substituted by C₁₋₆ alkyl group, and containing, besides carbon atoms, 1 to 3 heteroatoms selected from the group consisting of nitrogen atom, oxygen atom and sulfur atom.

### [Compound (Ig)]

A compound represented by

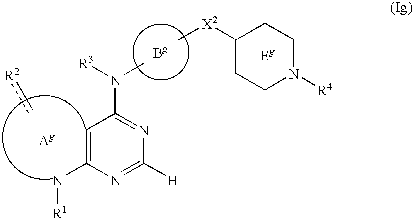

wherein ring A^{g} is an optionally further substituted nitrogen-containing 7-membered or 8-membered ring, ring B^{g} is an optionally further substituted phenyl group or an optionally further substituted pyridyl group, ring E^{g} is an optionally further substituted piperidyl group, R⁴ is a hydrogen atom or an acyl group, X² is a group represented by —O—, —S—, —SO—, —SO₂—, —CH₂— or —CO—NR⁵— (wherein R⁵ is a hydrogen atom, or an optionally substituted C₁₋₆ alkyl group), other symbols are as defined above, and R³ is optionally bonded to the carbon atom on ring B^{g} to form an optionally substituted ring structure, or a salt thereof.

In the above-mentioned formula, as the “optionally further substituted nitrogen-containing 7-membered or 8-membered ring” represented by ring A^{g}, those similar to the above-mentioned “optionally further substituted nitrogen-containing 7-membered or 8-membered ring” represented by ring A^{a} can be used.

As the further substituent of the “optionally further substituted piperidyl group” represented by ring E^{g}, for example, 1 to 4, same or different substituents selected from the above-mentioned substituent group V can be used.

As the further substituent of the “optionally further substituted phenyl group” or “optionally further substituted pyridyl group” represented by ring B^{g}, for example, 1 to 4, same or different substituents selected from the above-mentioned substituent group V can be used.

As the ring B^{g}, an “optionally further substituted phenyl group” is preferable.

As the “acyl group” represented by R⁴, those mentioned above can be used.

As the “optionally substituted ring structure” formed by R^{3g} bonded to the carbon atom on ring B^{g}, those similar to the “optionally substituted ring structure” formed by R³ bonded to the carbon atom on ring B can be used.

### [Compound (Ih)]

A compound represented by

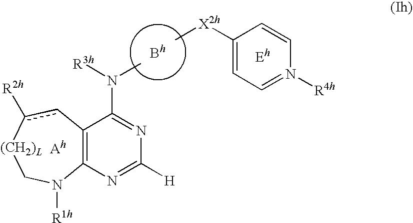

wherein ring A^{h} is an optionally further substituted nitrogen-containing 7-membered or 8-membered ring, L is 1 or 2,
formula [symbol] is a single bond or a double bond, ring B^{h} is an optionally further substituted phenyl group or an optionally further substituted pyridyl group,
ring E^{h} is an optionally further substituted piperidyl group,
X^{2h} is a group represented by —O—, —S—, —SO—, —SO₂—, —CH₂— or —CO—NR^{5h}— (wherein R^{5h} is a hydrogen atom, or an optionally substituted C₁₋₆ alkyl group),
R^{1h} is a hydrogen atom, or an optionally substituted group bonded via a carbon atom or a sulfur atom,
R^{2h} is a hydrogen atom, or an optionally substituted group bonded via a carbon atom, a nitrogen atom, an oxygen atom or a sulfur atom,
R^{3h} is a hydrogen atom, or an optionally substituted aliphatic hydrocarbon group, or R^{3h} is optionally bonded to the carbon atom on ring B^{h} to form an optionally substituted ring structure, and
R^{4h} is a hydrogen atom or an acyl group, or a salt thereof.

Specific examples of the “nitrogen-containing 7-membered or 8-membered ring” of the “optionally further substituted nitrogen-containing 7-membered or 8-membered ring” for ring A^{h} include the 7-membered ring and 8-membered ring shown by the following structures:

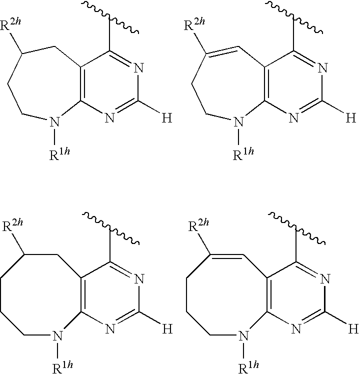

wherein each symbol is as defined above.

The “nitrogen-containing 7-membered or 8-membered ring” of the “optionally further substituted nitrogen-containing 7-membered or 8-membered ring” for ring A^{h} optionally has, besides R^{1h} group and R^{2h} group, 1 to 3 substituents at substitutable position(s). As such substituent, for example, a substituent selected from the above-mentioned substituent group V can be mentioned. When the number of the substituents is not less than 2, respective substituents may be the same or different.

As the further substituent of the “optionally further substituted piperidyl group” represented by ring E^{h}, those similar to ring E^{g} can be used.

As the further substituent of the “optionally further substituted phenyl group” or “optionally, further substituted pyridyl group” represented by ring B^{h}, those similar to ring B^{e} can be used. Of those, a halogen atom is preferable.

As the ring B^{h}, a phenyl group optionally substituted by halogen atom is preferable.

As X^{2h}, those similar to X² can be used. When X^{2h} is —CONR^{5h}—, as the substituent of the “optionally substituted C₁₋₆ alkyl group” represented by R^{5h}, those similar to R⁵ can be used.

As “—X^{2h}—”, —O— is preferable.

As the “optionally substituted group bonded via a carbon atom or a sulfur atom” represented by R^{1h}, those similar to the “optionally substituted group bonded via a carbon atom or a sulfur atom” represented by R¹ can be used.

As R^{1h}, a hydrogen atom is preferable.

As the “optionally substituted group bonded via a carbon atom, a nitrogen atom, an oxygen atom or a sulfur atom” represented by R^{2h}, those similar to the “optionally substituted group bonded via a carbon atom, a nitrogen atom, an oxygen atom or a sulfur atom” represented by R² can be used.

As R^{2h},

(i) a C₁₋₆ alkyl group optionally substituted by hydroxy,
(ii) a C₁₋₆ alkoxy-carbonyl group,
(iii) a group represented by —CO—NR^{e}R^{f}

wherein R^{e} is a hydrogen atom or a C₁₋₆ alkyl group.
R^{f} is a group represented by
   (a) a C₁₋₆ alkyl group optionally substituted by 1 or 2 substituents selected from the group consisting of
      (1) hydroxy,
      (2) amino,
      (3) cyano,
      (4) amino mono- or di-substituted by C₁₋₆ alkyl optionally having hydroxy,
      (5) C₁₋₆ alkylcarbonyl-amino,
      (6) C₁₋₆ alkoxy optionally having hydroxy,
      (7) C₁₋₆ alkoxy-carbonyl,
      (8) C₁₋₆ alkylsulfonyl,
      (9) 5- to 8-membered heterocyclyl-sulfonyl containing, besides carbon atoms, 1 to 3 heteroatoms selected from the group consisting of nitrogen atom, oxygen atom and sulfur atom,
      (10) 5- to 8-membered heterocyclic group optionally having 1 or 2 substituents selected from the group consisting of C₁₋₆ alkyl, C₆₋₁₈ aryl, and C₆₋₁₈ aryl-C₁₋₆ alkyl, and containing, besides carbon atoms, 1 to 3 heteroatoms selected from the group consisting of nitrogen atom, oxygen atom and sulfur atom,
      (11) C₆₋₁₈ aryl-sulfonyl, and
      (12) C₆₋₁₈ aryl group optionally having 1 or 2 halogens atom,
   (b) a C₃₋₇ cycloalkyl group,
   (c) a C₁₋₆ alkoxy group, or
   (d) a 5- to 8-membered heterocyclic group optionally having C₁₋₆ alkyl or C₆₋₁₈ aryl-C₁₋₆ alkyl, and containing, besides carbon atoms, 1 to 3 heteroatoms selected from the group consisting of nitrogen atom, oxygen atom and sulfur atom, or
      (iv) a 5- to 8-membered cyclic amino-carbonyl group optionally substituted by substituents selected from the group consisting of

(a) hydroxy,

(b) C₁₋₆ alkylsulfonyl, and

(c) C₁₋₆ alkyl optionally having C₁₋₆ alkylsulfonyl is preferable.

As the “acyl group” for R^{4h}, those similar to R⁴ can be used.

As R^{4h}, a C₃₋₇ cycloalkyl-carbonyl group or a C₁₋₆ alkoxy-carbonyl group is preferable.

As the substituent of the “optionally substituted aliphatic hydrocarbon group” represented by R^{3h}, those similar to R³ can be used.

As R^{3h}, a hydrogen atom is preferable.

As the “optionally substituted ring structure” formed by R^{3h} bonded to the carbon atom on ring B^{h}, those similar to the “optionally substituted ring structure” formed by R³ bonded to the carbon atom on ring B can be used.

A preferable embodiment of compound (Ih) is compound (Ih)

wherein
ring A^{h} is a nitrogen-containing 7-membered or 8-membered ring (preferably 7-membered ring) without a substituent other than R^{1h} and R^{2h},
L is 1 or 2 (preferably 1),
R^{1h} is a hydrogen atom,

### R^{2h} is

(i) a C₁₋₆ alkyl group optionally substituted by hydroxy,
(ii) a C₁₋₆ alkoxy-carbonyl group,
(iii) a group represented by —CO—NR^{e}R^{f}

wherein R^{e} is a hydrogen atom or a C₁₋₆ alkyl group, and
R^{f} is
   (a) a C₁₋₆ alkyl group optionally substituted by the 1 or 2 substituents selected from the group consisting of
      (1) hydroxy,
      (2) amino,
      (3) cyano,
      (4) amino mono- or di-substituted by C₁₋₆ alkyl optionally having hydroxy,
      (5) C₁₋₆ alkylcarbonyl-amino,
      (6) C₁₋₆ alkoxy optionally having hydroxy,
      (7) C₁₋₆ alkoxy-carbonyl,
      (8) C₁₋₆ alkylsulfonyl,
      (9) 5- to 8-membered heterocycle containing, besides carbon atoms, 1 to 3 heteroatoms selected from the group consisting of nitrogen atom, oxygen atom and sulfur atom-sulfonyl,
      (10) 5- to 8-membered heterocyclic group optionally having 1 or 2 substituents selected from the group consisting of C₁₋₆ alkyl, C₆₋₁₈ aryl, and C₆₋₁₈ aryl-C₁₋₆ alkyl, and containing, besides carbon atoms, 1 to 3 heteroatoms selected from the group consisting of nitrogen atom, oxygen atom and sulfur atom,
      (11) C₆₋₁₈ aryl-sulfonyl, and
      (12) C₆₋₁₈ aryl group optionally having 1 or 2 halogens atom,
   (b) a C₃₋₇ cycloalkyl group,
   (c) a C₁₋₆ alkoxy group, or
   (d) a 5- to 8-membered heterocyclic group optionally having C₁₋₆ alkyl or C₆₋₁₈ aryl-C₁₋₆ alkyl, and containing, besides carbon atoms, 1 to 3 heteroatoms selected from the group consisting of nitrogen atom, oxygen atom and sulfur atom, or
      (iv) a 5- to 8-membered cyclic amino-carbonyl group optionally substituted by substituents selected from the group consisting of

(a) hydroxy,

(b) C₁₋₆ alkylsulfonyl, and

(c) C₁₋₆ alkyl optionally having C₁₋₆ alkylsulfonyl,

R^{3h} is a hydrogen atom,
ring B^{h} is a phenyl group optionally further substituted by a halogen atom,
X^{2h} is a group represented by —O—, and
R^{4h} is 5- to 8-membered heterocyclyl-carbonyl group optionally having 1 or 2 substituents selected from the group consisting of (1) C₃₋₇ cycloalkyl-carbonyl group, (2) C₁₋₆ alkoxy-carbonyl group and (3) C₁₋₆ alkyl, and containing, besides carbon atoms, 1 to 3 heteroatoms selected from the group consisting of nitrogen atom, oxygen atom and sulfur atom.

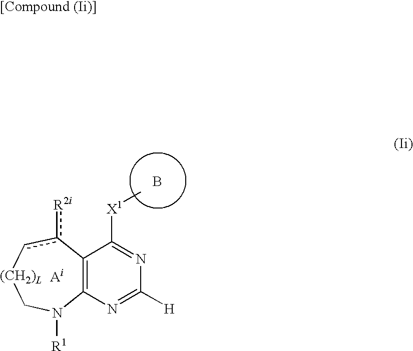

wherein ring Aⁱ is an optionally further substituted nitrogen-containing 7-membered or 8-membered ring, L is 1 or 2, formula [symbol] is a single bond or a double bond, R²ⁱ is, a hydrogen atom, or an optionally substituted group bonded via a carbon atom, a nitrogen atom, an oxygen atom or a sulfur atom when [symbol] R²ⁱ is —R²ⁱ, and an oxo group, an optionally substituted alkylidene group, or an optionally substituted imino group when [symbol] R²ⁱ is ═R²ⁱ, other symbols are as defined above, and R²ⁱ and R³ may be bonded to each other to form an optionally substituted ring structure.

In the above-mentioned formula, as the “optionally substituted group bonded via a carbon atom, a nitrogen atom, an oxygen atom or a sulfur atom” represented by R²ⁱ, those similar to the “optionally substituted group bonded via a carbon atom, a nitrogen atom, an oxygen atom or a sulfur atom” represented by R² can be used.

As R²ⁱ, a group represented by ═O, ═CH—CO—OR⁶ⁱ, ═CH—CO—NHR⁶ⁱ, ═CH—CO—NR⁶ⁱR⁷ⁱ, ═CH—CH₂—OR⁶ⁱ, ═CH—CH₂—NHR⁶ⁱ, ═CH—CH₂—NR⁶ⁱR⁷ⁱ, ═CH—CH₂—S(O)_{q}R⁶ⁱ or ═N—O—R⁶ⁱ [wherein R⁶ⁱ and R⁷ⁱ are the same or different and each is a hydrogen atom or an optionally substituted C₁₋₆ alkyl group, and q is 1 or 2], is preferable.

Here, as the “optionally substituted C₁₋₆ alkyl group” represented by R⁶ⁱ or R⁷ⁱ, those similar to the “optionally substituted C₁₋₆ alkyl group” represented by R⁵ can be used. Alternatively, when R²ⁱ is ═CH—CO—NR⁶ⁱR⁷ⁱ or ═CH—CH₂—NR⁶ⁱR⁷ⁱ, R⁶ⁱ and R⁷ⁱ may form nitrogen-containing heterocycle together with the adjacent nitrogen atom.

As the “nitrogen-containing heterocycle”, for example, a 3- to 8-membered nitrogen-containing heterocycle containing, as a ring constituent atom besides carbon atom, at least 1 nitrogen atom, and optionally further having 1 or 2 heteroatoms selected from the group consisting of oxygen atom, sulfur atom and nitrogen atom can be mentioned. As the preferable examples of the nitrogen-containing heterocycle, 5- or 6-membered cyclic amino optionally containing oxygen atom (e.g., 1-pyrrolidinyl, piperidinyl, 1-piperazinyl, morpholinyl) can be mentioned.

The “nitrogen-containing heterocycle” of the “optionally substituted nitrogen-containing heterocycle” optionally has 1 to 3 substituents at substitutable position (s). As such substituent, for example, a substituent selected from the above-mentioned substituent group V can be mentioned. When the number of the substituents is not less than 2, respective substituents may be the same or different.

Specific examples of the fused ring of the “nitrogen-containing 7-membered or 8-membered ring” of the “optionally further substituted nitrogen-containing 7-membered or 8-membered ring” for ring Aⁱ and a pyrimidine ring include the following structures:

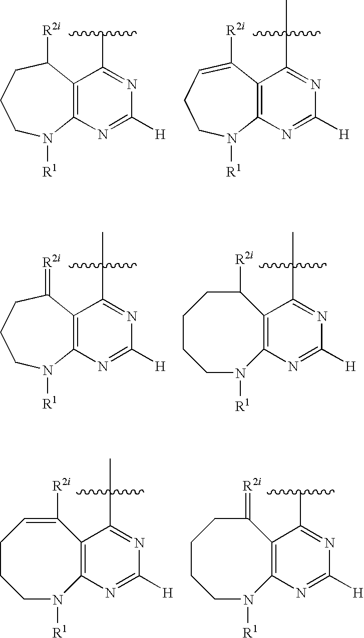

wherein each symbol is as defined above.

The “nitrogen-containing 7-membered or 8-membered ring” of the “optionally further substituted nitrogen-containing 7-membered or 8-membered ring” for ring Aⁱ optionally has, besides R¹¹ group and R²ⁱ group, 1 to 3 substituents at substitutable position (s). As such substituent, for example, a substituent selected from the above-mentioned substituent group V can be mentioned. When the number of the substituents is not less than 2, respective substituents may be the same or different.

As the “ring structure” when R²ⁱ and R³ are bonded to each other to form an optionally substituted ring structure, a saturated or unsaturated (preferably saturated) 4- to 8-membered (preferably 5- to 7-membered) heterocycle can be mentioned. When R²ⁱ and R³ are bonded to each other to form an optionally substituted ring structure, for example,

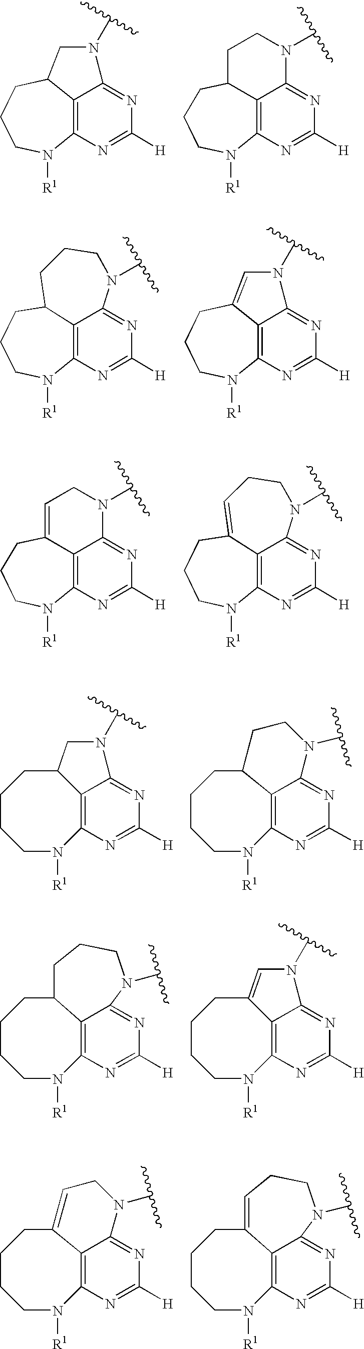

wherein each symbol is as defined above, and the like can be mentioned.

The “ring structure” formed by R²ⁱ and R³ are bonded to each other may have 1 to 5 (preferably 1 to 3, more preferably 1 or 2), same or different substituents selected from the above-mentioned substituent group V, at any substitutable positions.

As compound (I), the following compound or a salt thereof is particularly preferable.
4-({3-chloro-4-[3-(trifluoromethyl)phenoxy]phenyl}amino)-N-[3-(1H-imidazol-1-yl)propyl]-8,9-dihydro-7H-pyrimido[4,5-b]azepin-6-carboxamide,
4-({3-chloro-4-[3-(trifluoromethyl)phenoxy]phenyl}amino)-N-[2-(2-hydroxyethoxy)ethyl]-8,9-dihydro-7H-pyrimido[4,5-b]azepin-6-carboxamide,
4-({3-chloro-4-[3-(trifluoromethyl)phenoxy]phenyl}amino)-N-[2-(2-methoxyethoxy)ethyl]-8,9-dihydro-7H-pyrimido[4,5-b]azepin-6-carboxamide,
methyl 4-[(4-{3-[(tert-butylamino)carbonyl]phenoxy}-3-chlorophenyl)amino]-8,9-dihydro-7H-pyrimido[4,5-b]azepine-6-carboxylate,
4-({3-chloro-4-[3-(trifluoromethyl)phenoxy]phenyl}amino)-N-(2,3-dihydroxypropyl)-8,9-dihydro-7H-pyrimido[4,5-b]azepine-6-carboxamide,
4-{[3-chloro-4-(3-chlorophenoxy)phenyl]amino}-N-[2-(2-hydroxyethoxy)ethyl]-8,9-dihydro-7H-pyrimido[4,5-b]azepin-6-carboxamide,
tert-butyl 4-(2-chloro-4-{[6-(hydroxymethyl)-8,9-dihydro-7H-pyrimido[4,5-b]azepin-4-yl]amino}phenoxy)piperidine-1-carboxylate,
4-({3-chloro-4-[3-(trifluoromethyl)phenoxy]phenyl}amino)-N-{2-[(2-hydroxyethyl)sulfonyl]ethyl}-8,9-dihydro-7H-pyrimido[4,5-b]azepin-6-carboxamide,
4-({3-chloro-4-[3-(trifluoromethyl)phenoxy]phenyl}amino)-N-{2-[(2-hydroxyethyl)sulfonyl]-1,1-dimethylethyl}-8,9-dihydro-7H-pyrimido[4,5-b]azepin-6-carboxamide,
N-(tert-butyl)-3-[2-chloro-4-({6-[({2-[(2-hydroxyethyl)sulfonyl]-1,1-dimethylethyl}amino)methyl]-8,9-dihydro-7H-pyrimido[4,5-b]azepin-4-yl}amino)phenoxy]benzamide,
methyl 4-[(6-{3-[(tert-butylamino)carbonyl]phenoxy}-5-chloropyridin-3-yl)amino]-8,9-dihydro-7H-pyrimido[4,5-b]azepin-6-carboxylate,
4-[(3-chloro-4-{3-[(cyclopropylmethyl)sulfonyl]phenoxy}phenyl)amino]-N-{2-[(2-hydroxyethyl)sulfonyl]-1,1-dimethylethyl}-8,9-dihydro-7H-pyrimido[4,5-b]azepin-6-carboxamide,
N-(tert-butyl)-3-{2-chloro-4-[(6-{[(2-hydroxyethoxy)imino]methyl}-8,9-dihydro-7H-pyrimido[4,5-b]azepin-4-yl)amino]phenoxy}benzamide,
N-(tert-butyl)-3-{2-chloro-4-[(6-{[(2-fluoroethyl)amino]methyl}-8,9-dihydro-7H-pyrimido[4,5-b]azepin-4-yl)amino]phenoxy}benzamide,
N-(tert-butyl)-3-(2-chloro-4-{[6-({methyl[2-(methylsulfonyl)ethyl]amino}methyl)-8,9-dihydro-7H-pyrimido[4,5-b]azepin-4-yl]amino}phenoxy)benzamide, or
ethyl (2E)-3-{4-[(4-{3-[(tert-butylamino) carbonyl]phenoxy}-3-chlorophenyl)amino]-8,9-dihydro-7H-pyrimido[4,5-b]azepin-6-yl}acrylate.

As a salt of the compounds represented by each of the aforementioned formulas, for example, metal salts, ammonium salts, salts with organic bases, salts with inorganic acids, salts with organic acids, salts with basic or acidic amino acids and the like can be mentioned.

As preferable examples of the metal salts, alkali metal salts such as sodium salt, potassium salt and the like; alkaline earth metal salts such as calcium salt, magnesium salt, barium salt and the like; aluminum salt and the like can be mentioned.

As preferable examples of the salts with organic

bases, salts with trimethylamine, triethylamine, pyridine, picoline, 2,6-lutidine, ethanolamine, diethanolamine, triethanolamine, tromethamine[tris(hydroxymethyl)methylamine], t-butylamine, cyclohexylamine, dicyclohexylamine, N,N′-dibenzylethylenediamine and the like can be mentioned.

As preferable examples of the salts with inorganic acids, salts with hydrochloric acid, hydrobromic acid, nitric acid, sulfuric acid, phosphoric acid and the like can be mentioned.

As preferable examples of the salts with organic acids, salts with formic acid, acetic acid, trifluoroacetic acid, phthalic acid, fumaric acid, oxalic acid, tartaric acid, maleic acid, citric acid, succinic acid, malic acid, methanesulfonic acid, benzenesulfonic acid, p-toluenesulfonic acid and the like can be mentioned.

As preferable examples of the salts with basic amino acids, salts with arginine, lysine, ornithine and the like can be mentioned.

As preferable examples of the salts with acidic amino acids, salts with, aspartic acid, glutamic acid and the like can be mentioned.

Of those, pharmaceutically acceptable salts are preferable. For example, when a compound has an acidic functional group therein, salts with inorganic bases such as alkali metal salts (e.g., sodium salt, potassium salt and the like), alkaline earth metal salts (e.g., calcium salt, magnesium salt, barium salt and the like) and the like, ammonium salt and the like can be mentioned. When a compound has a basic functional group therein, salts with inorganic acids such as hydrochloric acid, hydrobromic acid, nitric acid, sulfuric acid, phosphoric acid and the like, and salts with organic acids such as acetic acid, phthalic acid, fumaric acid, oxalic acid, tartaric acid, maleic acid, citric acid, succinic acid, methanesulfonic acid, p-toluenesulfonic acid and the like can be mentioned.

Compound (I) of the present invention can be obtained by, for example, the method shown by the following scheme or a method analogous thereto and the like.

Each compound in the following schemes includes salts, and as such salts, for example, those similar to the salts of compound (I) and the like can be used.

The compound obtained in each step can be used as a reaction mixture or as a crude product in the next reaction. In addition, the compound can be isolated from a reaction mixture according to a conventional method, and can be easily purified by a separation means such as recrystallization, distillation, chromatography and the like.

Schematic reaction formulas are shown in the following, wherein each symbol of the compounds is as defined above.

Compound (I) of the present invention can be produced, for example, by reacting a compound represented by the formula:

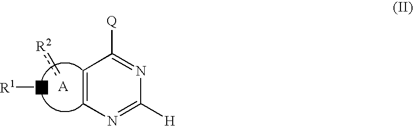

wherein Q is a leaving group, and other symbols are as defined above, or a salt thereof and a compound represented by the formula:

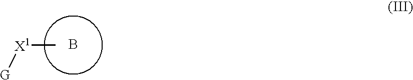

wherein G is a hydrogen atom or a metal atom, and other symbols are as defined above, or a salt thereof.

When X¹ is —NR³—Y¹—, —O— or —S—, G is mainly a hydrogen atom, but may be an alkali metal such as lithium, sodium, potassium, cesium and the like, or an alkaline earth metal such as magnesium, calcium and the like. When X¹ is —CHR³—, G is preferably a metal such as lithium, halogenated magnesium, copper, zinc and the like.

Compound (III) or a salt thereof is preferably used in an amount of 1 to 5 equivalent, preferably 1 to 2 equivalent, relative to compound (II) and the reaction is preferably carried out in a solvent. In addition, a base or an ammonium salt may be used in an amount of about 1 to 10 equivalent, preferably 1 to 2 equivalent.

In the aforementioned formula, as the leaving group for Q, a halogen atom such as chlorine, bromine, iodine and the like, a group represented by the formula —S(O)ₖR^{a} (wherein k is 0, 1 or 2, R^{a} is a lower (C₁₋₄)alkyl group such as methyl, ethyl, propyl and the like), benzyl group, a C₆₋₁₀ aryl group such as phenyl, tolyl and the like, and the like.] or a group represented by the formula —OR^{a} (wherein R^{a} is as defined above.) can be used.

As the solvent in the aforementioned reaction, for example, halogenated hydrocarbons such as dichloromethane, chloroform, carbon tetrachloride, 1,2-dichloroethane and the like, aromatic hydrocarbons such as benzene, toluene, xylene and the like, alcohols such as methanol, ethanol, isopropanol, t-butanol and the like, ethers such as diethyl ether, tetrahydrofuran, dioxane and the like, acetone, acetonitrile, ethyl acetate, N,N-dimethylformamide, N,N-dimethylacetamide, 1-methyl-2-pyrrolidone, dimethyl sulfoxide, hexamethylphosphoramide, water, a mixed solvent thereof and the like can be used.

As the base in the aforementioned reaction, an inorganic base, an organic base and the like can be used. Specifically, for example, sodium hydroxide, potassium hydroxide, sodium carbonate, potassium carbonate, sodium hydrogen carbonate, potassium hydrogen carbonate, triethylamine, N-ethyldiisopropylamine, pyridine, N,N-dimethylaminopyridine, sodium methoxide, sodium ethoxide, potassium t-butoxide, sodium hydride, sodium amide, diazabicycloundecene (DBU) and the like can be used.

As the ammonium salt in the aforementioned reaction, pyridine hydrochloride, pyridine hydrobromide, pyridinium p-toluenesulfonate, quinoline hydrochloride, isoquinoline hydrochloride, pyrimidine hydrochloride, pyrazine hydrochloride, triazine hydrochloride, trimethylamine hydrochloride, triethylamine hydrochloride, N-ethyldiisopropylamine hydrochloride and the like can be used.

The aforementioned reaction can be carried out under cooling, at room temperature or under heating (about 40 to 200° C., preferably about 40 to 160° C.), and the reaction time is generally about 1 to 30 hr, preferably about 1 to 20 hr, more preferably about 1 to 10 hr.

Compound (I) wherein X¹ is —SO— or —SO₂— can be produced by subjecting compound (I) wherein X¹ is —S— to an oxidization reaction. As the oxidant therefor, for example, metachloroperbenzoic acid, hydrogen peroxide, peroxyacetic acid, t-butylhydroperoxide, peroxysulfuric acid potassium, potassium permanganate, sodium perboronate, sodium periodate, sodium hypochlorite, halogen and the like can be used. When compound (I) wherein X¹ is —SO— is to be produced, the oxidant is used in an about 1 to 1.5 equivalent amount relative to the starting compound, and when compound (I) wherein X¹ is —SO₂— is to be produced, it is used in an about 2 to 3 equivalent amount. The reaction solvent is hot particularly limited as long as it does not react with the oxidant and, for example, halogenated hydrocarbons such as dichloromethane, chloroform, carbon tetrachloride, 1,2-dichloroethane and the like, aromatic hydrocarbons such as benzene, toluene, xylene and the like, alcohols such as methanol, ethanol, isopropanol, t-butanol and the like, ethers such as diethyl ether, tetrahydrofuran, dioxane and the like, carboxylic acids such as acetic acid, trifluoroacetic acid and the like, acetonitrile, ethyl acetate, N,N-dimethylformamide, N,N-dimethylacetamide, 1-methyl-2-pyrrolidone, dimethyl sulfoxide, water, a mixed solvent thereof and the like can be used. The reaction can be carried out under cooling, at room temperature or under heating, reaction time is generally about 1 to 20 hr, preferably about 1 to 10 hr.

A compound within the scope of the present invention can be also produced by applying means known per se to the obtained compound (I) of the present invention for introduction of substituents and conversion of functional groups. For conversion of substituents, a known conventional method can be used. For example, conversion to carboxy group by hydrolysis of ester, conversion to carbamoyl group by amidation of carboxy group, conversion to hydroxymethyl group by reduction of carboxy group, conversion to alcohol compound by reduction or alkylation of carbonyl group, reductive amination of carbonyl group, oximation of carbonyl group, acylation of amino group, alkylation of amino group, substitution and amination of active halogen by amine, alkylation of hydroxy group, substitution and amination of hydroxy group and the like can be mentioned. When a reactive substituent that causes non-objective reaction is present during the introduction of substituents and conversion of functional groups, a protecting group is introduced in advance as necessary into the reactive substituent by a means known per se, and the protecting group is removed by a means known per se after the objective reaction, whereby the compound within the scope of the present invention can be also produced.

As the starting compound (III) of this production method, a commercially available one is used or can be produced by a means known per se.

The starting compound (II) of this production method can be produced by, for example, a method shown by the following scheme. Here, compounds (IIa), (IIb), (IIc), (IId) and (IIe) are encompassed in compound (II).

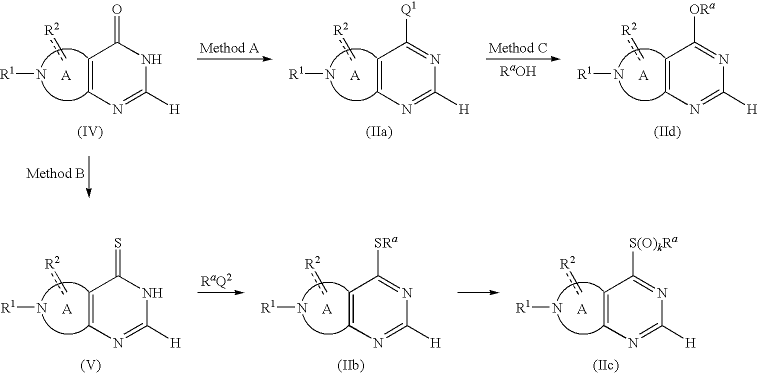

wherein Q¹ and Q² are each a halogen atom, k is 1 or 2, and other symbols are as defined above.

As Method A, compound (IIa) can be produced by reacting compound (IV) with a halogenating agent. As Method B, compound (IV) is reacted with a thionating agent to give compound (V), which is then reacted with a compound represented by R^{a}Q² in the presence of a base to give compound (IIb), which is further subjected to an oxidation reaction to give compound (IIc). As Method C, compound (IIa) is reacted with a compound represented by R^{z}OH in the presence of a base to give compound (IId).

As the halogenating agent in Method A, for example, about 1 to 100 equivalent of phosphorus oxychloride, phosphorus pentachloride, phosphorus trichloride, thionyl chloride, sulfuryl chloride, phosphorus tribromide and the like can be used. In this case, the reaction may be carried out in the presence of a base such as diethylaniline, dimethylaniline, pyridine and the like. While the reaction may be carried out without solvent, as a reaction solvent, for example, halogenated hydrocarbons such as dichloromethane, chloroform, carbon tetrachloride, 1,2-dichloroethane and the like; aromatic hydrocarbons such as benzene, toluene, xylene and the like; ethers such as diethyl ether, tetrahydrofuran, dioxane and the like; acetonitrile, ethyl acetate and the like may be used. The reaction is carried out under cooling, at room temperature or under heating, and the reaction time is generally about 1 to 20 hr, preferably about 1 to 10 hr.

As the thionating agent used in the production step from compound (IV) to compound (V) in Method B, for example, about 1 to 5 equivalent of a Lawesson reagent, phosphorus pentasulfide and the like can be used. As the reaction solvent, for example, halogenated hydrocarbons such as dichloromethane, chloroform, carbon tetrachloride, 1,2-dichloroethane and the like; aromatic hydrocarbons such as benzene, toluene, xylene and the like; ethers such as diethyl ether, tetrahydrofuran, dioxane and the like; and the like can be used. The reaction is carried out at room temperature or under heating, and the reaction time is generally about 1 to 20 hr, preferably about 1 to 10 hr.

As R^{a}Q² in the production step from compound (V) to compound (IIb) in Method B, for example, about 1 to 5 quivalent of methyl iodide, benzyl chloride, benzyl bromide and the like can be used, and as the base, for example, sodium hydroxide, potassium hydroxide, sodium carbonate, potassium carbonate, sodium hydrogencarbonate, potassium hydrogencarbonate, triethylamine, N-ethyldiisopropylamine, pyridine, N,N-dimethylaminopyridine, sodium methoxide, sodium ethoxide, potassium t-butoxide, sodium hydride, sodium amide, diazabicycloundecene (DBU) and the like can be used. As the reaction solvent, for example, halogenated hydrocarbons such as dichloromethane, chloroform, carbon tetrachloride, 1,2-dichloroethane and the like; aromatic hydrocarbons such as benzene, toluene, xylene and the like; alcohols such as methanol, ethanol, isopropanol, t-butanol and the like; ethers such as diethyl ether, tetrahydrofuran, dioxane and the like; acetone, acetonitrile, ethyl acetate, N,N-dimethylformamide, N,N-dimethylacetamide, 1-methyl-2-pyrrolidone, dimethyl sulfoxide, hexamethylphosphoramide, water or a mixed solvent thereof and the like can be used. The reaction is carried out under cooling, at room temperature or under heating, and the reaction time is generally about 1 to 20 hr, preferably about 1 to 10 hr.

As the oxidizing agent in the production step from compound (IIb) to compound (IIc) in Method B, for example, m-chloroperbenzoic acid, hydrogen peroxide, peracetic acid, t-butyl hydroperoxide, potassium peroxysulfate, potassium permanganate, sodium perborate, sodium periodate, sodium hypochlorite, halogen and the like can be used. When compound (IIc) wherein k=1 is produced, the oxidizing agent is used in about 1 to 1.5 equivalent relative to compound (IIb), and when compound (IIc) wherein k=2 is produced, it is used in about 2 to 3 equivalent relative to compound (IIb). The reaction solvent is not particularly limited as long as it does not react with the oxidizing agent and, for example, halogenated hydrocarbons such as dichloromethane, chloroform, carbon tetrachloride, 1,2-dichloroethane and the like; aromatic hydrocarbons such as benzene, toluene, xylene and the like; alcohols such as methanol, ethanol, isopropanol, t-butanol and the like; ethers such as diethyl ether, tetrahydrofuran, dioxane and the like; carboxylic acids such as acetic acid, trifluoroacetic acid and the like; acetonitrile, ethyl acetate, N,N-dimethylformamide, N,N-dimethylacetamide, 1-methyl-2-pyrrolidone, dimethyl sulfoxide, water or a mixed solvent thereof and the like can be used. The reaction is carried out under cooling, at room temperature or under heating, and the reaction time is generally about 1 to 20 hr, preferably about 1 to 10 hr.

As R^{a}OH in the production step from compound (IIa) to compound (IId) in Method C, for example, about 1 to 10 equivalent of methanol, ethanol, phenol and the like can be used, and as the base, for example, sodium hydroxide, potassium hydroxide, sodium carbonate, potassium carbonate, sodium hydrogencarbonate, potassium hydrogencarbonate, triethylamine, N-ethyldiisopropylamine, pyridine, N,N-dimethylaminopyridine, sodium methoxide, sodium ethoxide, potassium t-butoxide, sodium hydride, sodium amide, diazabicycloundecene (DBU) and the like can be used. As a reaction solvent, for example, halogenated hydrocarbons such as dichloromethane, chloroform, carbon tetrachloride, 1,2-dichloroethane and the like; aromatic hydrocarbons such as benzene, toluene, xylene and the like; ethers such as diethyl ether, tetrahydrofuran, dioxane and the like; acetone, acetonitrile, ethyl acetate, N,N-dimethylformamide, N,N-dimethylacetamide, 1-methyl-2-pyrrolidone, dimethyl sulfoxide, hexamethylphosphoramide, water or a mixed solvent thereof and the like can be used. The reaction is carried out under cooling, at room temperature or under heating, and the reaction time is generally about 1 to 20 hr, preferably about 1 to 10 hr.

Depending on the kind of the substituent of starting compound (II), a starting compound (II) having a different substituent can be produced by substituent conversion from, as a starting material, a compound produced by the above-mentioned production method. For the substituent conversion, a known general method can be used. For example, conversion to carbamoyl group by hydrolysis and amidation of ester, conversion to hydroxymethyl group by reduction of carboxy group, conversion to alcohol compound by reduction or alkylation of carbonyl group, reductive amination of carbonyl group, oximation of carbonyl group, acylation of amino group, alkylation of amino group, substitution and amination of active halogen by amine, alkylation of hydroxy group, substitution and amination of hydroxy group and the like can be mentioned. When a reactive substituent that causes non-objective reaction is present during the introduction of substituents and conversion of functional groups, a protecting group is introduced in advance as necessary into the reactive substituent by a means known per se, and the protecting group is removed by a means known per se after the objective reaction, whereby the starting compound (II) can be also produced.

Compound (I) can also be produced by subjecting a compound represented by the formula

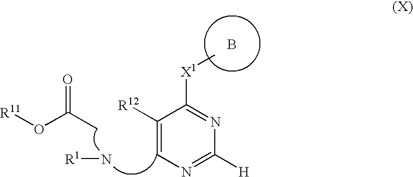

wherein R¹¹ is a C₁₋₆ alkyl group, R¹² is a group represented by —CHO or —COOR¹³ (wherein R¹³ is a C₁₋₆ alkyl group), and other symbols are as defined above, or a salt thereof to an intramolecular dehydrating condensation reaction in the presence of a base and, where necessary, subjecting the compound to a substituent conversion reaction.

For example, a compound represented by the formula

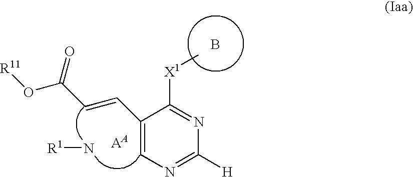

wherein ring A^{A} is an optionally further substituted azepin ring or an optionally substituted azocine ring, and other symbols are as defined above, or a salt thereof [hereinafter sometimes to be referred to as compound (Iaa)] can be produced by the method shown by the next formula. In the formula, the “azepin ring” or “azocine ring” of the “optionally substituted azepin ring” or “optionally substituted azocine ring” for ring A^{A} optionally has, besides “R¹¹—O—CO— group” and R¹ group, 1 to 3 substituents at substitutable position(s).

As such substituent, for example, the substituent selected from the above-mentioned Substituent group V can be mentioned. When the number of the substituents is not less than 2, respective substituents may be the same or different.

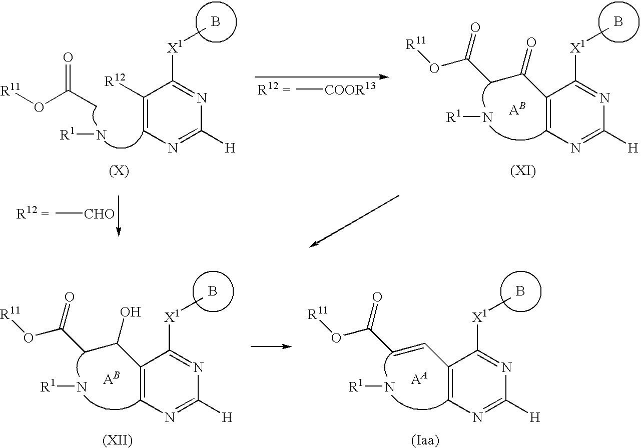

wherein ring A^{B} is an optionally further substituted nitrogen-containing 7-membered or 8-membered ring, and other symbols are as defined above.

In the formula, the “nitrogen-containing 7-membered or 8-membered ring” of the “optionally further substituted nitrogen-containing 7-membered or 8-membered ring” for ring A^{B} optionally has, besides “R¹¹—O—CO— group” and R¹ group, 1 to 3 substituents at substitutable position(s). As such substituent, for example, the substituent selected from above-mentioned Substituent group V can be mentioned. When the number of the substituents is not less than 2, respective substituents may be the same or different.

Compound (X¹) and compound (XII) can be produced by subjecting compound (X) to a Dieckmann (type) condensation reaction (J. P. Schaefer and J. J. Bloomfield, Org. Reactions, 1967, 15, 1). Compound (XII) can be produced by subjecting compound (X¹) to catalytic hydrogenation or reduction reaction using sodium borohydride and the like. Compound (Iaa) can be produced by subjecting compound (XII) to dehydrating reaction by a conventional method.

In compound (X), when R¹² is “—CHO”, compound (Iaa) can be directly produced from compound (X) by reaction the compound using dimethyl carbonate, DMF and the like as a reaction solvent, in the presence of 1 to 5 equivalent of a base (sodium methoxide, sodium hydride, etc.) at 0 to 100° C. (preferably 20 to 60° C.) for 1 to 100 hr (preferably 1 to 50 hr).

In addition, compound (Iaa) can be produced, for example, by reacting starting material compound (IIaa) or (IIab), that can be produced by a method shown in the following formula, with compound (III).

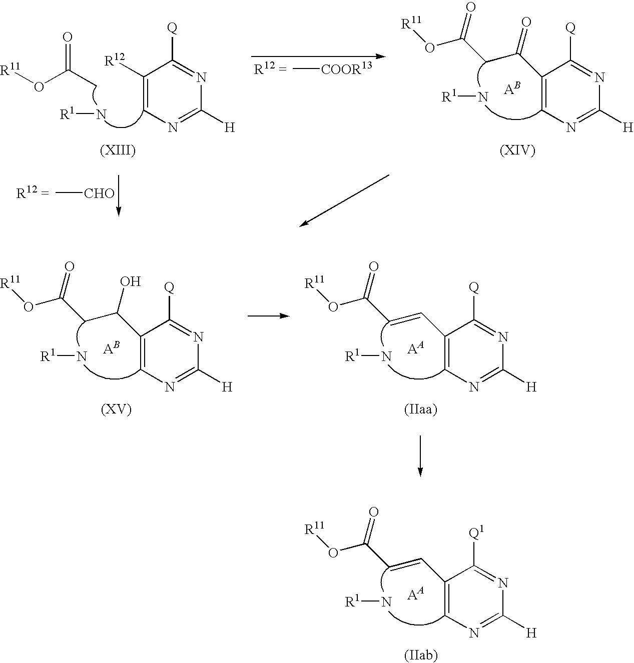

wherein each symbol is as defined above.

Compound (XIV) or compound (XV) can be produced by subjecting compound (XIII) to a Dieckmann (type) condensation reaction (J. P. Schaefer and J. J. Bloomfield, Org. Reactions, 1967, 15, 1). Compound (XV) can be produced by subjecting compound (XIV) to catalytic hydrogenation or reduction reaction using sodium borohydride and the like. Compound (IIaa) can be produced by subjecting compound (XV) to dehydrating reaction by a conventional method.

Compound (XV) can be directly produced from compound (XIII) by reacting compound (XIII) wherein R¹² is “—CHO” in dimethyl carbonate, DMF and the like as a reaction solvent in the presence of 1 to 5 equivalent of a base (sodium methoxide, sodium hydride, etc.) at 0 to 100° C. (preferably 20 to 60° C.) for 1 to 100 hr (preferably 1 to 50 hr).

Compound (IIab) can be produced by reacting compound (IIaa), wherein Q is an alkoxy group, with a halogenating agent (phosphoryl chloride, etc.) without solvent or in the presence of a solvent such as 1,2-dimethoxyethane, 1,2-dichloroethane and the like at room temperature to 150° C. for 1 to 200 hr, preferably 50 to 150 hr.

Compound (Iaa) can be produced by reacting compound (IIaa) or compound (IIab) with compound (III) according to a method for producing compound (I) by reacting the aforementioned compound (II) with compound (III) or a modification thereof.

A compound within the range of the present invention can be produced by introducing a substituent into the obtained compound (Iaa) or converting a functional group thereof by a method known per se. For example, by subjecting —CO—O—R¹¹ group, which is a substituent on ring A^{A} of compound (Iaa), to substituent conversion or conversion of functional group, various compounds shown below can be produced.

### [Production Method of Amide Compound [Compound (Iac)]]

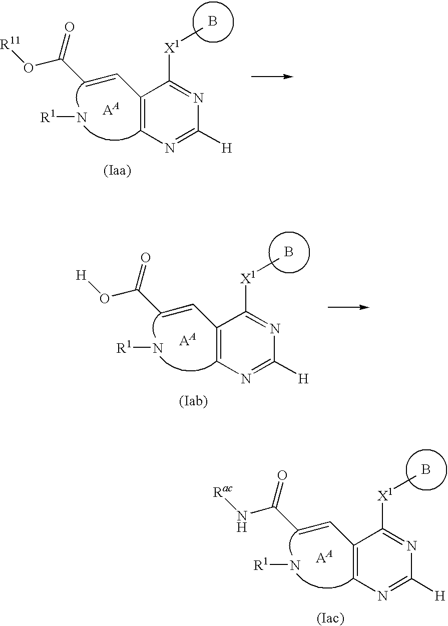

wherein R^{ac} is an optionally substituted hydrocarbon group, an optionally substituted heterocyclic group or an acyl group, and other symbols are as defined above.

—CO—O—R¹¹ group of ring A^{A} of compound (Iaa) is hydrolyzed by a method known per se to lead to carboxylic acid, whereby compound (Iab) is obtained, which is then reacted with an amine derivative to give compound (Iac), which is an amide derivative. A condensation reaction of the carboxylic acid derivative and the amine derivative is performed by peptide synthesis by a method known per se.

### [Production Method of Alcohol Compound [Compound (Iad)]]

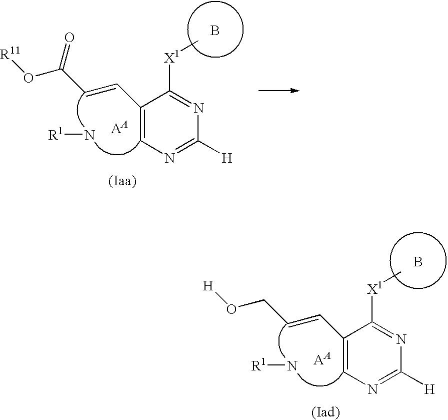

wherein each symbol is as defined above.

Compound (Iad), which is an alcohol derivative, can be synthesized by a general reduction reaction using sodium borohydride and the like, using an ester compound (Iaa) or carboxylic acid obtained by hydrolysis thereof or a mixed acid anhydride or acid halide derived therefrom as a starting material,

### [Production Method of Ether Compound [Compound (Iaf)]]

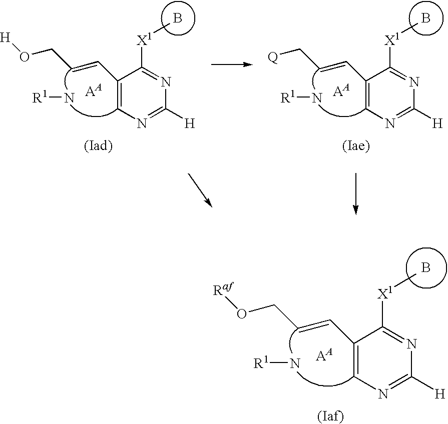

wherein R^{af} is an optionally substituted hydrocarbon group, an optionally substituted heterocyclic group, or an acyl group, and other symbols are as defined above.

Compound (Iaf) having ether can be produced by converting a hydroxy group of alcohol compound (Iad) to a leaving group Q by a method known per se to give compound (Iae), and converting the compound to an ether compound method by a known per se. Compound (Iaf) can also be directly produced from alcohol compound (Iad) under general etherification conditions.

### [Production Method of Amino Compound, Sulfide Compound, Sulfoxide Compound or Sulfone Compound [Compound (Iag)]]

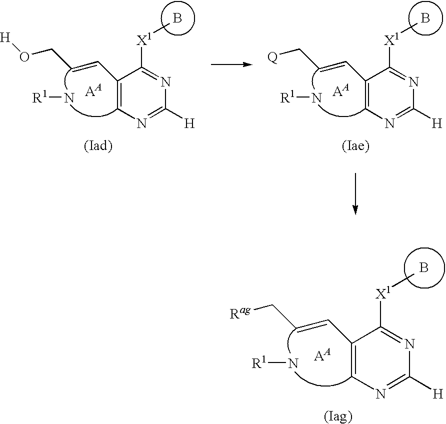

wherein R^{ag} is a group bonded via a nitrogen atom or a sulfur atom, and optionally substituted, and other symbols are as defined above.

A sulfide compound and an amino compound can also be produced by converting a hydroxy group of alcohol compound (Iad) to a leaving group Q, and then according to a method known per se. A sulfone compound and a sulfoxide compound can be produced by subjecting a sulfide compound to, for example, oxidation using peracid such as 3-chloroperbenzoic acid and the like or hydroperoxide and the like.

Compound (I), which is the resultant product of this reaction, may be produced as a single compound or in the form of a mixture.

Compound (I) of the present invention thus obtained can be isolated and purified at a high purity from a reaction mixture by a means known per se, for example, solvent extraction, concentration, neutralization, filtration, crystallization, recrystallization, column chromatography, high performance liquid chromatography and the like.

When compound (I) is obtained as a free form, it can be converted to a desired salt by a method known per se or a modification thereof; conversely, when compound (I) is obtained as a salt, it can be converted to a free form or other desired salt by a method known per se or a modification thereof.

When compound (I) has isomers such as optical isomer, stereoisomer, positional isomer, rotational isomer and the like, and any isomers and mixtures are encompassed in compound (I). For example, when compound (I) has an optical isomer, an optical isomer separated from a racemate is also encompassed in compound (I). These isomers can be obtained as independent products by a synthesis means or a separation means (concentration, solvent extraction, column chromatography, recrystallization and the like) known per se.

The compound (I) may be a crystal, and both a single crystal and crystal mixtures are encompassed in compound (I). Crystals can be produced by crystallization according to crystallization methods known per se.

Compound (I) may be a solvate (e.g., hydrate, etc.) or a non-solvate, both of which are encompassed in compound (I).

A compound labeled with an isotope (e.g., ³H, ¹⁴C, ³⁵S, ¹²⁵I and the like) is also encompassed in compound (I).

A prodrug of compound (I) or a salt thereof (hereinafter referred to as compound (I)) means a compound which is converted to compound (I) with a reaction due to an enzyme, an gastric acid, etc. under the physiological condition in the living body, that is, a compound which is converted to compound (I) with oxidation, reduction, hydrolysis, etc. according to an enzyme; a compound which is converted to compound (I) by hydrolysis, etc. due to gastric acid, etc. A prodrug for compound (I) may be a compound obtained by subjecting an amino group in compound (I) to an acylation, alkylation or phosphorylation (e.g., a compound obtained by subjecting an amino group in compound (I) to an eicosanoylation, alanylation, pentylaminocarbonylatioh, (5-methyl-2-oxo-1,3-dioxolen-4-yl)methoxycarbonylation, tetrahydrofuranylation, pyrrolidylmethylation, pivaloyloxymethylation and tert-butylation, etc.); a compound obtained by subjecting a hydroxy group in compound (I) to an acylation, alkylation, phosphorylation or boration (e.g., a compound obtained by subjecting an hydroxy group in compound (I) to an acetylation, palmitoylation, propanoylation, pivaloylation, succinylation, fumarylation, alanylation, dimethylaminomethylcarbonylation, etc.); a compound obtained by subjecting a carboxyl group in compound (I) to an esterification or amidation (e.g., a compound obtained by subjecting a carboxyl group in compound (I) to an ethyl esterification, phenyl esterification, carboxymethyl esterification, dimethylaminomethyl esterification, pivaloyloxymethyl esterification, ethoxycarbonyloxyethyl esterification, phthalidyl esterification, (5-methyl-2-oxo-1,3-dioxolen-4-yl)methyl esterification, cyclohexyloxycarbonylethyl esterification and methylamidation, etc.) and the like. These compounds can be produced from compound (I) by a method known per se.

A prodrug for compound (I) may also be one which is converted into compound (I) under a physiological condition, such as those described in IYAKUHIN no KAIHATSU (Development of Pharmaceuticals), Vol. 7, Design of Molecules, p. 163-198, Published by HIROKAWA SHOTEN (1990).

The compound (I) of the present invention, or a salt thereof or a prodrug thereof (hereinafter referred to as the compound of the present invention) possesses tyrosine kinase-inhibiting activity and can be used for the prophylaxis or treatment of tyrosine kinase-dependent diseases in mammals. Tyrosine kinase-dependent diseases include diseases characterized by increased cell proliferation due to abnormal tyrosine kinase enzyme activity. Furthermore, the compound of the present invention specifically inhibits HER2 kinase and/or EGFR kinase and is therefore also useful as a therapeutic agent for suppressing the growth of HER2 and/or EGFR kinase-expressing cancer, or a preventive agent for preventing the transition of hormone-dependent cancer to hormone-independent cancer. In addition, the compound is useful as a pharmaceutical agent because it shows low toxicity (e.g., acute toxicity, chronic toxicity, genetic toxicity, reproductive toxicity, cardiotoxicity, drug interaction, carcinogenicity and the like), high water solubility, and is superior in stability, pharmacokinetics (absorption, distribution, metabolism, excretion and the like) and efficacy expression.

That is, the compound of the present invention can be used as a safe agent for the prophylaxis or treatment of diseases caused by abnormal cell growth such as various cancers (particularly breast cancer (including progressive breast cancer, for example, invasive ductal carcinoma, ductal cancer in situ, inflammatory breast cancer, etc.), prostate cancer (e.g., hormone-dependent prostate cancer, non-hormone dependent prostate cancer, etc.), pancreatic cancer (e.g., pancreatic duct cancer, etc.), gastric cancer (e.g., papillary adenocarcinoma, mucinous adenocarcinoma, adenosquamous cancer, etc.), lung cancer (e.g., non-small cell lung cancer, small cell lung cancer, malignant mesothelioma, etc.), colon cancer (e.g., gastrointestinal stromal tumor, etc.), colorectal cancer (e.g., familial colorectal cancer, hereditary nonpolyposis colorectal cancer, gastrointestinal stromal tumor, etc.), small intestine cancer, rectal cancer (e.g., gastrointestinal stromal tumor, etc.), esophagus cancer, duodenal cancer, cancer of tongue, cancer of pharynx (e.g., nasopharyngeal carcinoma, oropharyngeal cancer, hypopharyngeal cancer, etc.), brain tumor (e.g., pineal astrocytoma, pilocytic astrocytoma, diffuse astrocytoma, anaplastic astrocytoma, etc.), neurinoma, non-small cell lung cancer, small cell lung cancer, liver cancer (e.g., primary liver cancer, Extrahepatic Bile Duct Cancer, etc.), kidney cancer (e.g., renal cell carcinoma, renal pelvis and ureter transitional cell cancer, etc.), cancer of the bile duct, cancer of the uterine body, uterine cervical cancer, ovarian cancer (e.g., ovarian epithelial cancer, extragonadal germ cell tumor, ovarian germ cell tumor, ovarial low malignant potential tumor, etc.), urinary bladder cancer, skin cancer (e.g., ocular melanoma, Merkel cell carcinoma, etc.), hemangioma, malignant lymphoma, malignant melanoma, thyroid cancer (e.g., medullary thyroid cancer, etc.), bone tumor (e.g., osteosarcoma, Ewing's tumor, uterus sarcoma, soft tissue sarcoma, etc.), vascular fibroma, retinoblastoma, penile cancer, solid cancer in childhood, Kaposi's sarcoma, Kaposi's sarcoma derived from AIDS, maxillary tumor, fibrous histiocytoma, leiomyosarcoma, rhabdomyosarcoma, leukemia (e.g., acute myeloid leukemia, acute lymphoblastic leukemia, etc.), etc.), atherosclerosis, angiogenesis (e.g., angiogenesis associated with growth of solid cancer or sarcoma, angiogenesis associated with tumor metastasis, and angiogenesis associated with diabetic retinopathy, etc.), viral disease (HIV infection, etc.) and the like.

The tyrosine kinase-dependent disease further includes cardiovascular diseases associated with abnormal tyrosine kinase enzyme activity. Accordingly, the compound of the present invention can also be used as an agent for the prophylaxis or treatment of cardiovascular diseases such as restenosis.

The compound of the present invention is useful as an anti-cancer agent for the prophylaxis or treatment of cancer, particularly breast cancer (including progressive breast cancer), ovarian cancer, colorectal cancer, small intestine cancer, gastric cancer, esophagus cancer, prostate cancer, lung cancer, pancreatic cancer, kidney cancer, colon cancer, and the like.

The compound of the present invention shows low toxicity and can be used as a pharmaceutical agent as it is, or as a pharmaceutical composition in admixture with a commonly known pharmaceutically acceptable carrier, etc. in mammals (e.g., humans, horses, bovines, dogs, cats, rats, mice, rabbits, pigs, monkeys, and the like).

In addition to the compound of the present invention, other active ingredients, for example, the following hormonal therapeutic agents, anticancer agents (e.g., chemotherapeutic agents, immunotherapeutic agents and pharmaceutical agents inhibiting the action of cell growth factors or cell growth factor receptors), and the like may be contained in a pharmaceutical composition.

For administration of the compound of the present invention as a pharmaceutical agent for mammals such as humans, it can be administered orally in the form of, for example, tablets, capsules (including soft capsules and microcapsules), powders, granules and the like, or parenterally in the form of injections, suppositories, pellets and the like. Examples of the “parenteral administration” include intravenous administration, intramuscular administration, subcutaneous administration, intra-tissue administration, intranasal administration, intradermal administration, instillation administration, intracerebral administration, intrarectal administration, intravaginal administration, intraperitoneal administration, intratumoral administration, administration to the juxtaposition of tumor and administration directly to the lesion.

The dose of the compound of the present invention varies depending on the administration route, symptoms and the like. For example, when it is administered orally as an anticancer agent to a patient (body weight 40 to 80 kg) with breast cancer or prostate cancer, its dose is, for example, 0.5 to 100 mg/kg body weight per day, preferably 1 to 50 mg/kg body weight per day, and more preferably 1 to 25 mg/kg body weight per day. This amount may be administered once or in 2 or 3 divided portions daily.

The compound of the present invention can be safely administered orally or parenterally (e.g., topical, rectal, intravenous administrations, etc.) as a single agent, or a pharmaceutical composition containing a pharmacologically acceptable carrier such as tablet (including sugar-coated tablet, film-coated tablet), powder, granule, capsule, liquid, emulsion, suspension, injection, suppository, sustained release preparation, plaster and the like, according to a conventional method (e.g., a method described in the Japanese Pharmacopoeia, etc.).

Moreover, a combination of (1) administration of an effective amount of the compound of the present invention and (2) 1 to 3 kinds selected from the group consisting of (i) administration of an effective amount of other anticancer agents, (ii) administration of an effective amount of hormonal therapeutic agents and (iii) non-drug therapy can prevent and/or treat cancer more effectively. The non-drug therapy is exemplified by surgery, radiotherapy, gene therapy, thermotherapy, cryotherapy, laser cauterization, and the like, and two or more of these may be combined.

For example, the compound of the present invention can be administered to the same subject simultaneously with hormonal therapeutic agents, anticancer agents (e.g., chemotherapeutic agents, immunotherapeutic agents, or pharmaceutical agents inhibiting the action of cell growth factors or cell growth factor receptors) (hereafter to be briefly referred to as a concomitant drug).

Although the compound of the present invention exhibits excellent anticancer action even when used as a simple agent, its effect can be enhanced by using it in combination with one or more of the concomitant drug(s) mentioned above (multiple drug therapy).

Examples of the “hormonal therapeutic agents” include fosfestrol, diethylstylbestrol, chlorotrianisene, medroxyprogesterone acetate, megestrol acetate, chlormadinone acetate, cyproterone acetate, danazol, dienogest, asoprisnil, allylestrenol, gestrinone, nomegestrol, Tadenan, mepartricin, raloxifene, ormeloxifene, levormeloxifene, anti-estrogens (e.g., tamoxifen citrate, toremifene citrate, and the like), ER down regulator (e.g., fulvestrant, and the like), human menopausal gonadotrophin, follicle stimulating hormone, pill preparations, mepitiostane, testrolactone, aminoglutethimide, LH-RH agonists (e.g., goserelin acetate, buserelin, leuprorelin, and the like), droloxifene, epitiostanol, ethinylestradiol sulfonate, aromatase inhibitors (e.g., fadrozole hydrochloride, anastrozole, retrozole, exemestane, vorozole, formestane, and the like), anti-androgens (e.g., flutamide, bicartamide, nilutamide, and the like), 5α-reductase inhibitors (e.g., finasteride, dutasteride, epristeride, and the like), aderenal cortex hormone drugs (e.g., dexamethasone, prednisolone, betamethasone, triamcinolone, and the like), androgen synthesis inhibitors (e.g., abiraterone, and the like), retinoid and drugs that retard retinoid metabolism (e.g., liarozole, and the like), etc. and LH-RH agonists (e.g., goserelin acetate, buserelin, leuprorelin) are preferable.

Examples of the “chemotherapeutic agents” include alkylating agents, antimetabolites, anticancer antibiotics, plant-derived anticancer agents, and the like.

Examples of the “alkylating agents” include nitrogen mustard, nitrogen mustard-N-oxide hydrochloride, chlorambutyl, cyclophosphamide, ifosfamide, thiotepa, carboquone, improsulfan tosylate, busulfan, nimustine hydrochloride, mitobronitol, melphalan, dacarbazine, ranimustine, sodium estramustine phosphate, triethylenemelamine, carmustine, lomustine, streptozocin, pipobroman, etoglucid, carboplatin, cisplatin, miboplatin, nedaplatin, oxaliplatin, altretamine, ambamustine, dibrospidium hydrochloride, fotemustine, prednimustine, pumitepa, ribomustin, temozolomide, treosulphan, trophosphamide, zinostatin stimalamer, adozelesin, cystemustine, bizelesin, and the like.

Examples of the “antimetabolites” include mercaptopurine, 6-mercaptopurine riboside, thioinosine, methotrexate, enocitabine, cytarabine, cytarabine ocfosfate, ancitabine hydrochloride, 5-FU drugs (e.g., fluorouracil, tegafur, UFT, doxifluridine, carmofur, gallocitabine, emmitefur, and the like), aminopterine, leucovorin calcium, tabloid, butocine, folinate calcium, levofolinate calcium, cladribine, emitefur, fludarabine, gemcitabine, hydroxycarbamide, pentostatin, piritrexim, idoxuridine, mitoguazone, thiazophrine, ambamustine, and the like.

Examples of the “anticancer antibiotics” include actinomycin-D, actinomycin-C, mitomycin-C, chromomycin-A3, bleomycin hydrochloride, bleomycin sulfate, peplomycin sulfate, daunorubicih hydrochloride, doxorubicin hydrochloride, aclarubicin hydrochloride, pirarubicin hydrochloride, epirubicin hydrochloride, neocarzinostatin, mithramycin, sarcomycin, carzinophilin, mitotane, zorubicin hydrochloride, mitoxantrone hydrochloride, idarubicin hydrochloride, and the like.

Examples of the “plant-derived anticancer agents” include etoposide, etoposide phosphate, vinblastine sulfate, vincristine sulfate, vindesine sulfate, teniposide, paclitaxel, docetaxel, vinorelbine, and the like.

Examples of the “immunotherapeutic agents (BRM)” include picibanil, krestin, sizofuran, lentinan, ubenimex, interferons, interleukins, macrophage colony-stimulating factor, granulocyte colony-stimulating factor, erythropoietin, lymphotoxin, BCG vaccine, Corynebacterium parvum, levamisole, polysaccharide K, procodazole, and the like.

Example of the “cell growth factor” in the “pharmaceutical agents inhibiting the action of cell growth factors or cell growth factor receptors”, there may be mentioned any substances that promote cell proliferation, which are normally peptides having not more than 20,000 molecular weight that are capable of exhibiting their activity at low concentrations by binding to a receptor, including (1) EGF (epidermal growth factor) or substances possessing substantially the same activity as it [e.g., EGF, heregulin (HER2 ligand), and the like], (2) insulin or substances possessing substantially the same activity as it [e.g., insulin, IGF (insulin-like growth factor)-1, IGF-2, and the like], (3) FGF (fibroblast growth factor) or substances possessing substantially the same activity as it [e.g., acidic FGF, basic FGF, KGF (keratinocyte growth factor), FGF-10, and the like], (4) other cell growth factors [e.g., CSF (colony stimulating factor), EPO (erythropoietin), IL-2 (interleukin-2), NGF (nerve growth factor), PDGF (platelet-derived growth factor), TGF-β (transforming growth factor-β), HGF (hepatocyte growth factor), VEGF (vascular endothelial growth factor), and the like], and the like.

Examples of the “cell growth factor receptors” include any receptors capable of binding to the aforementioned growth factors, including EGF receptor, heregulin receptor (HER2), insulin receptor, IGF receptor, FGF receptor-1 or FGF receptor-2, and the like.

Examples of the “pharmaceutical agents inhibiting the action of cell growth factor” include trastuzumab (Herceptin (trade mark): HER2 antibody), imatinib mesylate, ZD1839 or cetuximab, antibody to VEGF (e.g., bevacizumab), antibody to VEGF receptor, gefitinib, erlotinib, and the like.

In addition to the aforementioned drugs, L-asparaginase, aceglatone, procarbazine hydrochloride, protoporphyrin-cobalt complex salt, mercuric hematoporphyrin-sodium, topoisomerase I inhibitors (e.g., irinotecan, topotecan, and the like), topoisomerase II inhibitors (e.g., sobuzoxane, and the like), differentiation inducers (e.g., retinoid, vitamin D, and the like), angiogenesis inhibitors (e.g., thalidomide, SU11248, and the like), α-blockers (e.g., tamsulosin hydrochloride, naftopidil, urapidil, alfuzosin, terazosin, prazosin, silodosin, and the like), serine/threonine kinase inhibitor, endothelin receptor antagonist (e.g., atrasentan, and the like), proteasome inhibitor (e.g., bortezomib, and the like), Hsp 90 inhibitors (e.g., 17-AAG, and the like), spironolactone, minoxidil, 11α-hydroxyprogesterone, bone resorption inhibiting/metastasis suppressing agent (e.g., zoledronic acid, alendronic acid, pamidronic acid, etidronic acid, ibandronic acid, clodronic acid) and the like can be used.

Of those mentioned above, LH-RH agonists (e.g., goserelin acetate, buserelin, leuprorelin, and the like), trastuzumab (HER2 antibody) and the like are preferable as concomitant drugs.

When the compound of the present invention and the concomitant drug are used in combination, the administration time of the compound of the present invention and the concomitant drug is not restricted, and the compound of the present invention and the concomitant drug can be administered to the subject simultaneously, or may be administered at different times. The dosage of the concomitant drug may be determined according to the dose clinically used, and can be appropriately selected depending on the subject, administration route, disease, combination of the drugs and the like.

The administration mode of the compound of the present invention and the concomitant drug is not particularly restricted, and the compound of the present invention and the concomitant drug only need to be combined at the time of administration. Examples of such administration mode include the following methods: (1) The compound of the present invention and the concomitant drug are simultaneously produced to give a single preparation which is administered. (2) The compound of the present invention and the concomitant drug are separately produced to give two kinds of preparations which are administered simultaneously by the same administration route. (3) The compound of the present invention and the concomitant drug are separately produced to give two kinds of preparations which are administered by the same administration route at different times. (4) The compound of the present invention and the concomitant drug are separately produced to give two kinds of preparations which are administered simultaneously by different administration routes. (5) The compound of the present invention and the concomitant drug are separately produced to give two kinds of preparations which are administered by different administration routes at different times (e.g., the compound of the present invention and the concomitant drug are administered in this order, or vice versa).

The present invention is explained in detail in the following by referring to Examples, Preparation Examples and Experimental Examples, which are not to be construed as limitative.

### EXAMPLES

### Reference Example 1

### Production of methyl 4-[(4-methoxybenzyl)amino]butanoate hydrochloride

Under hydrogen atmosphere, a mixture of 4-aminobutanoic acid (20.6 g), 4-methoxybenzaldehyde (29.9 g), 10% palladium-carbon (4.0 g), ethanol (300 mL) and 1N aqueous sodium hydroxide (200 mL) was stirred at room temperature for 7 days. Palladium-carbon was removed by filtration, and ethanol was evaporated under reduced pressure. Tetrahydrofuran (200 mL) was added to the residue, and di-tert-butyl dicarbonate (43 g) was added thereto dropwise at room temperature. The mixture was stirred at room temperature for 3 hr and extracted with hexane. The aqueous layer was acidified using 1N hydrochloric acid and extracted with diethyl ether. The organic layer was washed with saturated brine, dried over anhydrous magnesium sulfate and concentrated under reduced pressure to give 4-[(tert-butoxycarbonyl)(4-methoxybenzyl)amino]butanoic acid (62.7 g) as an orange oil. To a solution of 4-[(tert-butoxycarbonyl)(4-methoxybenzyl)amino]butanoic acid (62.7 g) in methanol (240 mL) was added dropwise thionyl chloride (47 mL) at −10° C. After the dropwise addition was completed, the mixture was stirred at room temperature for 24 hr, and concentrated under reduced pressure. Methanol was added to the residue and the mixture was further concentrated. The precipitated crystals were collected by filtration. The crystals were washed with diethyl ether to give the title compound (42.0 g) as colorless crystals.

¹H-NMR (DMSO-d₆) δ: 1.82-1.96 (2H, m), 2.44 (2H, t, J=7.4 Hz), 2.80-2.92 (2H, m), 3.60 (3H, s), 3.77 (3H, s), 4.04 (2H, t, J=5.7 Hz), 6.98 (2H, d, J=8.7 Hz), 7.47 (2H, d, J=8.7 Hz), 9.08-9.25 (2H, m).

### Reference Example 2

### Production of tert-butyl 4-[(4-methoxybenzyl)amino]butanoate

The mixture of tert-butyl 4-bromobutanoate (5.0 g) and 4-methoxybenzylamine (8.8 mL) was stirred at room temperature for 5 days. Aqueous sodium bicarbonate solution was added to the reaction mixture, and the mixture was extracted with ethyl acetate. The organic layer was washed with saturated brine, dried over anhydrous magnesium sulfate and concentrated under reduced pressure. The residue was separated and purified by column chromatography (hexane:ethyl acetate=3:1→2:1→1:1→ethyl acetate→ethyl acetate:methanol=19:1) to give the title compound (4.55 g) as a colorless oil.

¹H-NMR (CDCl₃) δ: 1.43 (9H, s), 1.74-1.83 (2H, m), 2.27 (2H, t, J=7.4 Hz), 2.63 (2H, t, J=7.1 Hz), 3.72 (2H, s), 3.80 (3H, s), 6.86 (2H, d, J=8.6 Hz), 7.23 (2H, d, J=8.6 Hz).

### Reference Example 3

### Production of methyl 5-[(4-methoxybenzyl)amino]pentanoate hydrochloride

Using 4-aminopentanoic acid (5.0 g), 4-methoxybenzaldehyde (6.39 g), 10% palladium-carbon (1.0 g), methanol (65 mL), 1N aqueous sodium hydroxide solution (43 ml), tetrahydrofuran (100 mL), di-tert-butyl dicarbonate (9.32 g), thionyl chloride (10.5 mL) and methanol (120 mL), a similar reaction as in Reference Example 1 was carried out to give the title compound (11.0 g) as colorless crystals.

¹H-NMR (DMSO-d₆) δ: 1.47-1.70 (4H, m), 2.34 (2H, t, J=6.9 Hz), 2.80-2.91 (2H, m), 3.59 (3H, s), 3.77 (3H, s), 4.04 (2H, s), 6.99 (2H, d, J=8.7 Hz), 7.44 (2H, d, J=8.7 Hz), 8.92 (2H, br s).

### Example 1

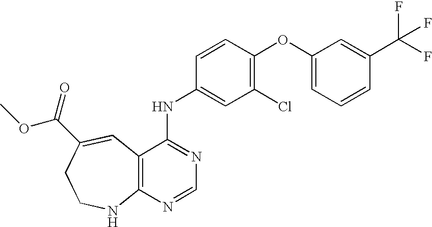

### Production of methyl 4-({3-chloro-4-[3-(trifluoromethyl)phenoxy]phenyl}amino)-8,9-dihydro-7H-pyrimido[4,5-b]azepine-6-carboxylate

### (i) Production of methyl 4-[(6-chloro-5-formylpyrimidin-4-yl)(4-methoxybenzyl)amino]butanoate

To a solution of 4,6-dichloropyrimidine-5-carbaldehyde (15.0 g) in acetonitrile (1500 mL) were successively added potassium phosphate (39.6 g) and methyl 4-[(4-methoxybenzyl)amino]butanoate hydrochloride (23.2 g), and the mixture was stirred at room temperature for 21 hr. The reaction mixture was concentrated under reduced pressure. To the residue was added water, and the mixture was extracted with ethyl acetate. The organic layer was washed with saturated brine, dried over anhydrous magnesium sulfate and concentrated under reduced pressure. The residue was separated and purified by silica gel column chromatography (eluent, hexane:ethyl acetate=95:5→67:33) to give the title compound (26.0 g) as a yellow oil.

¹H-NMR (CDCl₃) δ: 1.89-2.00 (2H, m), 2.30 (2H, t, J=7.3 Hz), 3.60-3.67 (2H, m), 3.65 (3H, s), 3.78 (3H, s), 4.55 (2H, s), 6.82 (2H, br d, J=8.8 Hz), 7.00 (2H, br d, J=8.8 Hz), 8.38 (1H, s), 10.19 (1H, s).

### (ii) Production of methyl 4-({3-chloro-4-[3-(trifluoromethyl)phenoxy]phenyl}amino)-9-(4-methoxybenzyl)-8,9-dihydro-7H-pyrimido[4,5-b]azepine-6-carboxylate

To a solution of methyl 4-[(6-chloro-5-formylpyrimidin-4-yl)(4-methoxybenzyl)amino]butanoate (11.0 g) in N,N-dimethylformamide (110 mL) were successively added sodium carbonate (3.10 g) and 3-chloro-4-[3-(trifluoromethyl)phenoxy]aniline (8.39 g), and the mixture was stirred at 60° C. for 108 hr. Water was added to the reaction mixture, and the mixture was extracted with ethyl acetate. The organic layer was washed with saturated brine, dried over anhydrous magnesium sulfate and concentrated under reduced pressure. The residue was separated and purified by silica gel column chromatography (eluent, hexane:ethyl acetate=90:10→50:50). The obtained oil was dissolved in dimethyl carbonate (230 mL). To the solution was added 28% sodium methoxide-methanol solution (12.3 g) and the mixture was stirred at room temperature for 15 hr. The reaction mixture was neutralized by IN hydrochloric acid and extracted with ethyl acetate. The organic layer was washed with saturated brine, dried over anhydrous magnesium sulfate and concentrated under reduced pressure. The residue was separated and purified by basic silica gel column chromatography (eluent, hexane:ethyl acetate=90:10→50:50) to give the title compound (8.44 g) as a yellow oil.

¹H-NMR (CDCl₃) δ: 2.66 (2H, t, J=4.6 Hz), 3.30-3.40 (2H, m), 3.78 (3H, s), 3.81 (3H, s), 4.87 (2H, s), 6.81-6.91 (3H, m), 7.03-7.14 (2H, m), 7.19-7.29 (3H, m), 7.30-7.36 (1H, m), 7.37-7.47 (2H, m), 7.68 (1H, s), 7.75 (1H, d, J=2.7 Hz), 8.24 (1H, s).

### (iii) Production of methyl 4-({3-chloro-4-[3-(trifluoromethyl)phenoxy]phenyl}amino)-8,9-dihydro-7H-pyrimido[4,5-b]azepine-6-carboxylate

To a solution of methyl 4-({3-chloro-4-[3-(trifluoromethyl)phenoxy]phenyl}amino)-9-(4-methoxybenzyl)-8,9-dihydro-7H-pyrimido[4,5-b]azepine-6-carboxylate (8.43 g) in 1,2-dichloroethane (85 mL) was added trifluoroacetic acid (85 mL), and the mixture was stirred at 70° C. for 14 hr. The reaction mixture was concentrated under reduced pressure. To the residue was added a saturated aqueous sodium hydrogen carbonate solution, and the mixture was extracted with ethyl acetate. The organic layer was washed with saturated brine, dried over anhydrous magnesium sulfate and concentrated under reduced pressure. The residue was separated and purified by silica gel column chromatography (eluent, hexane:ethyl acetate=80:20→0:100) to give the title compound (6.08 g) as a yellow powder.

¹H-NMR (CDCl₃) δ: 2.87-2.94 (2H, m), 3.48-3.58 (2H, m), 3.82 (3H, s), 5.89 (1H, t, J=4.5 Hz), 6.87 (1H, s), 7.06 (1H, d, J=8.8 Hz), 7.10 (1H, dd, J=7.7, 2.5 Hz), 7.21 (1H, s), 7.30-7.49 (3H, m), 7.67 (1H, s), 7.75 (1H, d, J=2.5 Hz), 8.13 (1H, s).

### Example 2

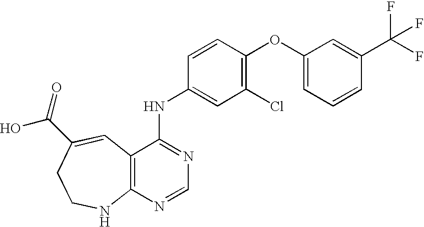

### Production of 4-({3-chloro-4-[3-(trifluoromethyl)phenoxy]phenyl}amino)-8,9-dihydro-7H-pyrimido[4,5-b]azepine-6-carboxylic acid

To a solution of methyl 4-({3-chloro-4-[3-(trifluoromethyl)phenoxy]phenyl}amino)-8,9-dihydro-7H-pyrimido[4,5-b]azepine-6-carboxylate (5.95 g) in tetrahydrofuran (170 mL) and ethanol (170 mL) was added 1N aqueous sodium hydroxide solution (40 mL), and the mixture was stirred at room temperature for 15 hr. To the reaction mixture was added 1N hydrochloric acid to adjust the pH to 3 to 4, and the mixture was diluted with water. The precipitate was filtered and dried to give the title compound (4.45 g) as a pale-yellow powder.

¹H-NMR (DMSO-d₆) δ: 2.65-2.78 (2H, m), 3.19-3.48 (2H, m), 7.13-7.29 (3H, m), 7.45 (1H, d, J=7.7 Hz), 7.51-7.64 (2H, m), 7.71 (1H, s), 7.84 (1H, d, J=2.5 Hz), 7.89 (1H, t, J=4.8 Hz), 7.99 (1H, s), 9.40 (1H, s).

### Example 3

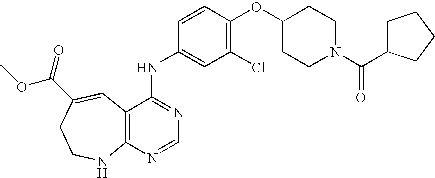

### Production of methyl 4-[(3-chloro-4-{[1-(cyclopentylcarbonyl)piperidin-4-yl]oxy}phenyl)amino]-8,9-dihydro-7H-pyrimido[4,5-b]azepine-6-carboxylate

### (i) Production of methyl 4-[(4-{[1-(tert-butoxycarbonyl)piperidin-4-yl]oxy}-3-chlorophenyl)amino]-9-(4-methoxybenzyl)-8,9-dihydro-7H-pyrimido[4,5-b]azepine-6-carboxylate

Using methyl 4-[(6-chloro-5-formylpyrimidin-4-yl)(4-methoxybenzyl)amino]butanoate (3.00 g), N,N-dimethylformamide (30 mL), sodium carbonate (843 mg), tert-butyl 4-(4-amino-2-chlorophenoxy)piperidine-1-carboxylate (2.60 g), 28% sodium methoxide-methanol solution (3.80 g) and dimethyl carbonate (80 mL), a similar reaction as in Example 1 (ii) was carried out to give the title compound (3.32 g) as an orange oil.

¹H-NMR (CDCl₃) δ: 1.48 (9H, s), 1.75-1.97 (4H, m), 2.65 (2H, t, J=4.5 Hz), 3.30-3.37 (2H, m), 3.37-3.48 (2H, m), 3.63-3.75 (2H, m), 3.77 (3H, s), 3.81 (3H, s), 4.43-4.52 (1H, m), 4.85 (2H, s), 6.70 (1H, s), 6.87 (2H, br d, J=8.8 Hz), 6.95 (1H, d, J=8.8 Hz), 7.23 (2H, br d, J=8.5 Hz), 7.29 (1H, dd, J=8.8, 2.7 Hz), 7.55 (1H, d, J=2.7 Hz), 7.66 (1H, s), 8.20 (1H, s).

### (ii) Production of methyl 4-[(4-{[1-(cyclopentylcarbonyl)piperidin-4-yl]oxy}-3-chlorophenyl)amino]-8,9-dihydro-7H-pyrimido[4,5-b]azepine-6-carboxylate

To a solution of methyl 4-[(4-{[1-(tert-butoxycarbonyl)piperidin-4-yl]oxy}-3-chlorophenyl)amino]-9-(4-methoxybenzyl)-8,9-dihydro-7H-pyrimido[4,5-b]azepine-6-carboxylate (3.31 g) in 1,2-dichloroethane (33 mL) was added trifluoroacetic acid (33 mL), and the mixture was stirred at 70° C. for 14 hr. The reaction mixture was concentrated under reduced pressure, and the residue was dissolved in tetrahydrofuran (30 mL) and cooled to 0° C. To the mixture were successively added triethylamine (5 mL) and cyclopentanecarbonyl chloride (0.62 mL), and the mixture was stirred at room temperature for 15 hr. Furthermore, cyclopentanecarbonyl chloride (0.31 mL) was added to the mixture and stirred for 2 hr. To the reaction mixture was added a saturated aqueous solution of sodium hydrogencarbonate, and the mixture was extracted with ethyl acetate. The organic layer was washed with saturated brine, dried over anhydrous magnesium sulfate and concentrated under reduced pressure. The residue was separated and purified by silica gel column chromatography (eluent, hexane:ethyl acetate=33:67→0:100→ethyl acetate:methanol=95:5), and further crystallized from diisopropyl ether/ethyl acetate to give the title compound (1.89 g) as a yellow powder.

¹H-NMR (CDCl₃) δ: 1.51-1.96 (12H, m), 2.86-2.98 (3H, m), 3.46-3.57 (3H, m), 3.71-3.79 (3H, m), 3.81 (3H, s), 4.49-4.64 (1H, m), 5.85 (1H, t, J=4.9 Hz), 6.75 (1H, s), 6.95 (1H, d, J=8.8 Hz), 7.24-7.35 (1H, m), 7.55 (1H, d, J=2.5 Hz), 7.66 (1H, m), 8.08 (1H, s).

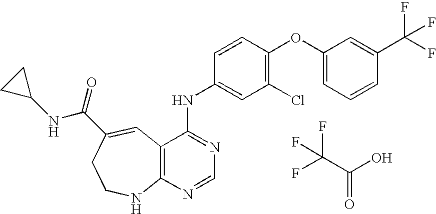

### Production of 4-({3-chloro-4-[3-(trifluoromethyl)phenoxy]phenyl}amino)-N-cyclopropyl-8,9-dihydro-7H-pyrimido[4,5-b]azepine-6-carboxamide trifluoroacetate

To a 0.1 M solution of 4-({3-chloro-4-[3-(trifluoromethyl)phenoxy]phenyl}amino)-8,9-dihydro-7H-pyrimido[4,5-b]azepine-6-carboxylic acid in N,N-dimethylformamide (0.5 mL) were successively added 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of cyclopropylamine in N,N-dimethylformamide (0.5 mL), and the mixture was stirred at room temperature for 21 hr. 5% Aqueous sodium hydrogen carbonate solution was added to the reaction mixture and the mixture was extracted with dichloromethane. The organic layer was dried and concentrated under reduced pressure. The residue was separated by preparative liquid chromatography (0.1% trifluoroacetic acid containing acetonitrile/0.1% trifluoroacetic acid containing water, 5%→100%) to collect the objective fraction, which was concentrated under reduced pressure to give the title compound (13.3 mg).

LC-MS found 516 (M+H)⁺ (exact MS calcd for C₂₅H₂₁ClF₃N₅O₂ 515).

### Example 5

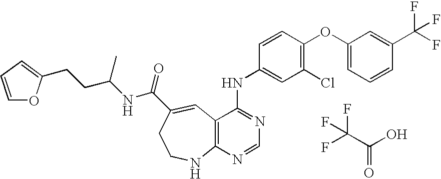

### Production of 4-({3-chloro-4-[3-(trifluoromethyl)phenoxy]phenyl}amino)-N-[3-(2-furyl)-1-methylpropyl]-8,9-dihydro-7H-pyrimido[4,5-b]azepine-6-carboxamide trifluoroacetate

Using 0.1 M solution of 4-({3-chloro-4-[3-(trifluoromethyl)phenoxy]phenyl}amino)-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of 4-(2-furyl)butan-2-amine in N,N-dimethylformamide (0.5 mL), a similar reaction as in Example 4 was carried out to give the title compound (27.4 mg).

LC-MS found 598 (M+H)⁺ (exact MS calcd for C₃₀H₂₇ClF₃N₅O₃ 597).

### Example 6

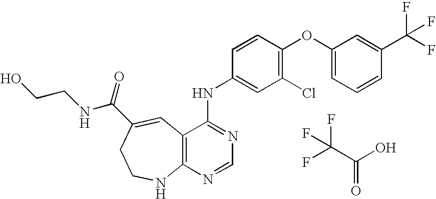

### Production of 4-({3-chloro-4-[3-(trifluoromethyl)phenoxy]phenyl}amino)-N-(2-hydroxyethyl)-8,9-dihydro-7H-pyrimido[4,5-b]azepine-6-carboxamide trifluoroacetate

Using 0.1 M solution of 4-({3-chloro-4-[3-(trifluoromethyl)phenoxy]phenyl}amino)-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of 2-aminoethanol in N,N-dimethylformamide (0.5 mL), a similar reaction as in Example 4 was carried out to give the title compound (27.4 mg).

LC-MS found 520 (M+H)⁺ (exact MS calcd for C₂₄H₂₁ClF₃N₅O₃ 519).

### Example 7

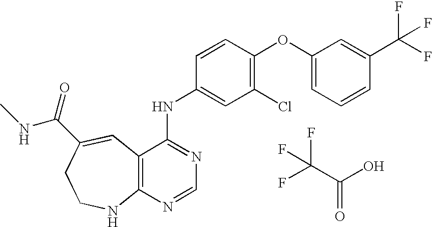

### Production of 4-({3-chloro-4-[3-(trifluoromethyl)phenoxy]phenyl}amino)-N-methyl-8,9-dihydro-7H-pyrimido[4,5-b]azepine-6-carboxamide trifluoroacetate

To 0.1 M solution of 4-({3-chloro-4-[3-(trifluoromethyl)phenoxy]phenyl}amino)-8,9-dihydro-7H-pyrimido[4,5-b]azepine-6-carboxylic acid in N,N-dimethylformamide (0.5 mL) were successively added 0.15 M solution of 1-hydroxybenzotriazole/1-ethyl-3-(3-dimethylaminopropyl)carbodiimide hydrochloride=1/1 in N,N-dimethylformamide (0.5 mL), 0.15 M solution of methylamine hydrochloride in N,N-dimethylformamide (0.5 mL) and triethylamine (0.01 mL), and the mixture was stirred at room temperature for 21 hr. To the reaction mixture was added 5% aqueous sodium hydrogen carbonate solution, and the mixture was extracted with dichloromethane. The organic layer was dried, and concentrated under reduced pressure. The residue was subjected to preparative liquid chromatography (0.1% trifluoroacetic acid containing acetonitrile/0.1% trifluoroacetic acid containing water, 5%→100%) to collect the objective fraction, and the fraction was concentrated under reduced pressure to give the title compound (19.9 mg).

LC-MS found 490 (M+H)⁺ (exact MS calcd for C₂₃H₁₉ClF₃N₅O₂ 489).

### Example 8

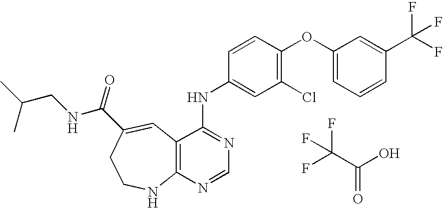

### Production of 4-({3-chloro-4-[3-(trifluoromethyl)phenoxy]phenyl}amino)-N-isobutyl-8,9-dihydro-7H-pyrimido[4,5-b]azepine-6-carboxamide trifluoroacetate

Using 0.1 M solution of 4-({3-chloro-4-[3-(trifluoromethyl)phenoxy]phenyl}amino)-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of isobutylamine in N,N-dimethylformamide (0.5 mL), a similar reaction as in Example 4 was carried out to give the title compound (25.0 mg).

LC-MS found 532 (M+H)⁺ (exact MS calcd for C₂₆H₂₅ClF₃N₅O₂ 531).

### Example 9

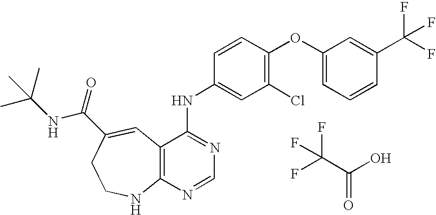

### Production of N-(tert-butyl)-4-({3-chloro-4-[3-(trifluoromethyl)phenoxy]phenyl}amino)-8,9-dihydro-7H-pyrimido[4,5-b]azepine-6-carboxamide trifluoroacetate

Using 0.1 M solution of 4-({3-chloro-4-[3-(trifluoromethyl)phenoxy]phenyl}amino)-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of tert-butylamine in N,N-dimethylformamide (0.5 mL), a similar reaction as in Example 4 was carried out to give the title compound (18.4 mg).

LC-MS found 532 (M+H)⁺ (exact MS calcd for C₂₆H₂₅ClF₃N₅O₂ 531).

### Example 10

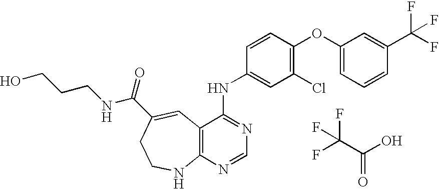

### Production of 4-({3-chloro-4-[3-(trifluoromethyl)phenoxy]phenyl}amino)-N-(3-hydroxypropyl)-8,9-dihydro-7H-pyrimido[4,5-b]azepine-6-carboxamide trifluoroacetate

Using 0.1 M solution of 4-({3-chloro-4-[3-(trifluoromethyl)phenoxy]phenyl}amino)-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of 3-amino-1-propanol in N,N-dimethylformamide (0.5 mL), a similar reaction as in Example 4 was carried out to give the title compound (26.2 mg).

LC-MS found 534 (M+H)⁺ (exact MS calcd for C₂₅H₂₃ClF₃N₅O₃ 533).

### Example 11

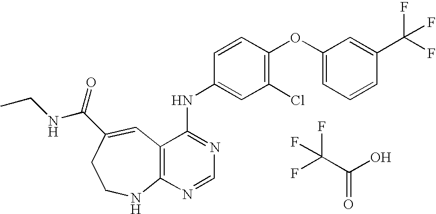

### Production of 4-({3-chloro-4-[3-(trifluoromethyl)phenoxy]phenyl}amino)-N-ethyl-8,9-dihydro-7H-pyrimido[4,5-b]azepine-6-carboxamide trifluoroacetate

Using 0.1 M solution of 4-({3-chloro-4-[3-(trifluoromethyl)phenoxy]phenyl}amino)-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL), 0.15 M solution of ethylamine hydrochloride in N,N-dimethylformamide (0.5 mL) and triethylamine (0.01 mL), a similar reaction as in Example 7 was carried out to give the title compound (23.8 mg).

LC-MS found 504 (M+H)⁺ (exact MS calcd for C₂₄H₂₁ClF₃N₅O₂ 503).

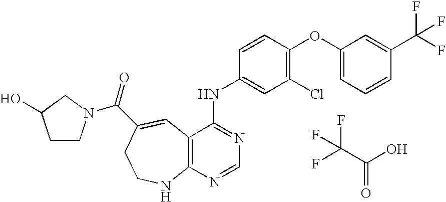

### Production of 1-{[4-({3-chloro-4-[3-(trifluoromethyl)phenoxy]phenyl}amino)-8,9-dihydro-7H-pyrimido[4,5-b]azepin-6-yl]carbonyl}pyrrolidin-3-ol trifluoroacetate

Using 0.1 M solution of 4-({3-chloro-4-[3-(trifluoromethyl)phenoxy]phenyl}amino)-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochlorides 1/1 in N,N-dimethylformamide (0.5 mL) and 0.15 M solution of pyrrolidin-3-ol in N,N-dimethylformamide (0.5 mL), a similar reaction as in Example 4 was carried out to give the title compound (27.1 mg).

LC-MS found 546 (M+H)⁺ (exact MS calcd for C₂₆H23ClF₃N₅O₃ 545).

### Example 13

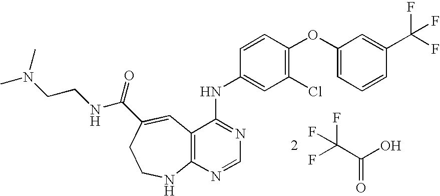

### Production of 4-({3-chloro-4-[3-(trifluoromethyl)phenoxy]phenyl}amino)-N-[2-(dimethylamino)ethyl]-8,9-dihydro-7H-pyrimido[4,5-b]azepine-6-carboxamide ditrifluoroacetate

Using 0.1 M solution of 4-({3-chloro-4-[3-(trifluoromethyl)phenoxy]phenyl}amino)-8,9-dihydro-7H-pyrimido [4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of N,N-dimethylethane-1,2-diamine in N,N-dimethylformamide (0.5 mL), a similar reaction as in Example 4 was carried out to give the title compound (32.9 mg).

LC-MS found 547 (M+H)⁺ (exact MS calcd for C₂₆H26ClF₃N₆O₂ 546).

### Example 14

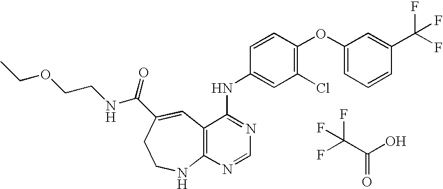

### Production of 4-({3-chloro-4-[3-(trifluoromethyl)phenoxy]phenyl}amino)-N-(2-ethoxyethyl)-8,9-dihydro-7H-pyrimido[4,5-b]azepine-6-carboxamide trifluoroacetate

Using 0.1 M solution of 4-({3-chloro-4-[3-(trifluoromethyl)phenoxy]phenyl}amino)-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of 2-ethoxyethylamine in N,N-dimethylformamide (0.5 mL), a similar reaction as in Example 4 was carried out to give the title compound (23.6 mg).

LC-MS found 548 (M+H)⁺ (exact MS calcd for C₂₆H₂₅ClF₃N₅O₃ 547).

### Example 15

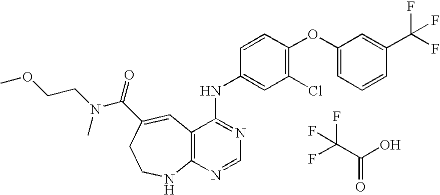

### Production of 4-({3-chloro-4-[3-(trifluoromethyl)phenoxy]phenyl}amino)-N-(2-methoxyethyl)-N-methyl-8,9-dihydro-7H-pyrimido[4,5-b]azepine-6-carboxamide trifluoroacetate

Using 0.1 M solution of 4-({3-chloro-4-[3-(trifluoromethyl)phenoxy]phenyl}amino)-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and, 0.15 M solution of N-(2-methoxyethyl)methylamine in N,N-dimethylformamide (0.5 mL), a similar reaction as in Example 4 was carried out to give the title compound (26.0 mg).

LC-MS found 548 (M+H)⁺ (exact MS calcd for C₂₆H₂₅ClF₃N₅O₃ 547).

### Example 16

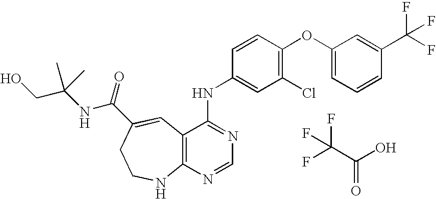

### Production of 4-({3-chloro-4-[3-(trifluoromethyl)phenoxy]phenyl}amino)-N-(2-hydroxy-1,1-dimethylethyl)-8,9-dihydro-7H-pyrimido[4,5-b]azepine-6-carboxamide trifluoroacetate

Using 0.1 M solution of 4-({3-chloro-4-[3-(trifluoromethyl)phenoxy]phenyl}amino)-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of 2-amino-2-methyl-1-propanol in N,N-dimethylformamide (0.5 mL), a similar reaction as in Example 4 was carried out to give the title compound (20.4 mg).

LC-MS found 548 (M+H)⁺ (exact MS calcd for C₂₆H₂₅ClF₃N₅O₃ 547).

### Example 17

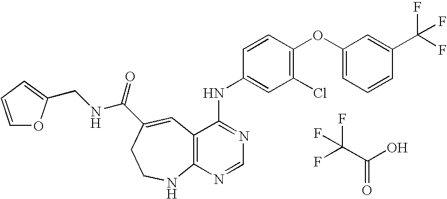

### Production of 4-({3-chloro-4-[3-(trifluoromethyl)phenoxy]phenyl}amino)-N-(2-furylmethyl)-8,9-dihydro-7H-pyrimido[4,5-b]azepine-6-carboxamide trifluoroacetate

Using 0.1 M solution of 4-({3-chloro-4-[3-(trifluoromethyl)phenoxy]phenyl}amino)-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of furfurylamine in N,N-dimethylformamide (0.5 mL), a similar reaction as in Example 4 was carried out to give the title compound (25.9 mg).

LC-MS found 556 (M+H)⁺ (exact MS calcd for C₂₇H₂₁ClF₃N₅O₃ 555).

### Example 18

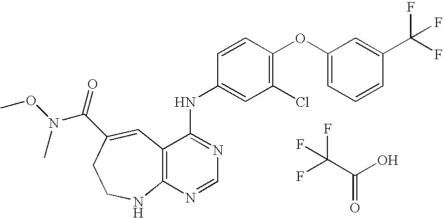

### Production of 4-({3-chloro-4-[3-(trifluoromethyl)phenoxy]phenyl}amino)-N-methoxy-N-methyl-8,9-dihydro-7H-pyrimido[4,5-b]azepine-6-carboxamide trifluoroacetate

Using 0.1 M solution of 4-({3-chloro-4-[3-(trifluoromethyl)phenoxy]phenyl}amino)-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL), 0.15 M solution of N,O-dimethylhydroxylamine hydrochloride in N,N-dimethylformamide (0.5 mL) and triethylamine (0.01 mL), a similar reaction as in Example 7 was carried out to give the title compound (24.5 mg).

LC-MS found 520 (M+H)⁺ (exact MS calcd for C₂₄H₂₁ClF₃N₅O₃ 519).

### Example 19

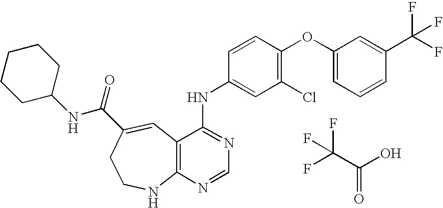

### Production of 4-({3-chloro-4-[3-(trifluoromethyl)phenoxy]phenyl}amino)-N-cyclohexyl-8,9-dihydro-7H-pyrimido[4,5-b]azepine-6-carboxamide trifluoroacetate

Using 0.1 M solution of 4-({3-chloro-4-[3-(trifluoromethyl)phenoxy]phenyl}amino)-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of cyclohexylamine in N,N-dimethylformamide (0.5 mL), a similar reaction as in Example 4 was carried out to give the title compound (25.0 mg).

LC-MS found 558 (M+H)⁺ (exact MS calcd for C₂₈H₂₇ClF₃N₅O₂ 557).

### Example 20

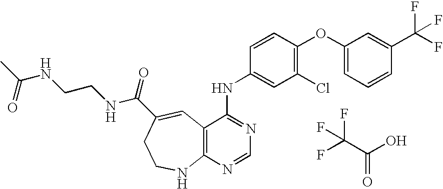

### Production of N-[2-(acetylamino)ethyl]-4-({3-chloro-4-[3-(trifluoromethyl)phenoxy]phenyl}amino)-8,9-dihydro-7H-pyrimido[4,5-b]azepine-6-carboxamide trifluoroacetate

Using 0.1 M solution of 4-({3-chloro-4-[3-(trifluoromethyl)phenoxy]phenyl}amino)-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of N-(2-aminoethyl)acetamide in N,N-dimethylformamide (0.5 mL), a similar reaction as in Example 4 was carried out to give the title compound (25.9 mg).

LC-MS found 561 (M+H)⁺ (exact MS calcd for C₂₆H₂₄ClF₃N₆O₃ 560).

### Example 21

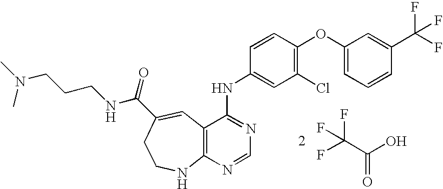

### Production of 4-({3-chloro-4-[3-(trifluoromethyl)phenoxy]phenyl}amino)-N-[3-(dimethylamino)propyl]-8,9-dihydro-7H-pyrimido[4,5-b]azepine-6-carboxamide ditrifluoroacetate

Using 0.1 M solution of 4-({3-chloro-4-[3-(trifluoromethyl)phenoxy]phenyl}amino)-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of N,N-dimethyl-1,3-propanediamine in N,N-dimethylformamide (0.5 mL), a similar reaction as in Example 4 was carried out to give the title compound (30.1 mg).

LC-MS found 561 (M+H)⁺ (exact MS calcd for C₂₇H₂₈ClF₃N₆O₂ 560).

### Example 22

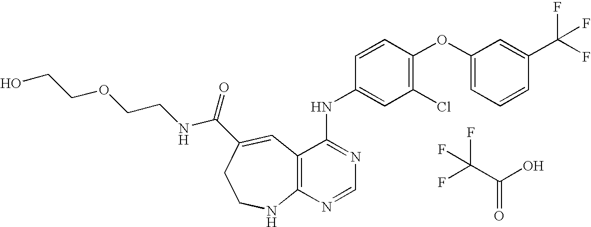

### Production of 4-({3-chloro-4-[3-(trifluoromethyl)phenoxy]phenyl}amino)-N-[2-(2-hydroxyethoxy)ethyl]-8,9-dihydro-7H-pyrimido[4,5-b]azepine-6-carboxamide trifluoroacetate

Using 0.1 M solution of 4-({3-chloro-4-[3-(trifluoromethyl)phenoxy]phenyl}amino)-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of 2-(2-aminoethoxy)ethanol in N,N-dimethylformamide (0.5 mL), a similar reaction as in Example 4 was carried out to give the title compound (27.1 mg).

LC-MS found 564 (M+H)⁺ (exact MS calcd for C₂₆H₂₅ClF₃N₅O₄ 563).

### Example 23

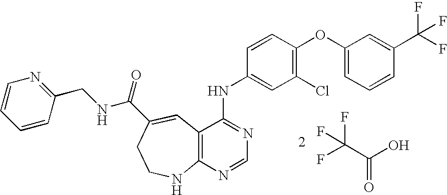

### Production of 4-({3-chloro-4-[3-(trifluoromethyl)phenoxy]phenyl}amino)-N-(pyridin-2-ylmethyl)-8,9-dihydro-7H-pyrimido[4,5-b]azepine-6-carboxamide ditrifluoroacetate

Using 0.1 M solution of 4-({3-chloro-4-[3-(trifluoromethyl)phenoxy]phenyl}amino)-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of 2-(aminomethyl)pyridine in N,N-dimethylformamide (0.5 mL), a similar reaction as in Example 4 was carried out to give the title compound (33.0 mg).

LC-MS found 567 (M+H)⁺ (exact MS calcd for C₂₈H₂₂ClF₃N₆O₂ 566).

### Example 24

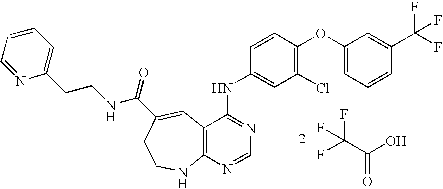

### Production of 4-({3-chloro-4-[3-(trifluoromethyl)phenoxy]phenyl}amino)-N-(2-pyridin-2-ylethyl)-8,9-dihydro-7H-pyrimido[4,5-b]azepine-6-carboxamide ditrifluoroacetate

Using 0.1 M solution of 4-({3-chloro-4-[3-(trifluoromethyl)phenoxy]phenyl}amino)-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of 2-(2-aminoethyl)pyridine in N,N-dimethylformamide (0.5 mL), a similar reaction as in Example 4 was carried out to give the title compound (35.9 mg).

LC-MS found 581 (M+H)⁺ (exact MS calcd for C₂₉H₂₄ClF₃N₆O₂ 580).

### Example 25

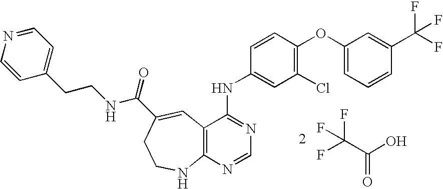

### Production of 4-({3-chloro-4-[3-(trifluoromethyl)phenoxy]phenyl}amino)-N-(2-pyridin-4-ylethyl)-8,9-dihydro-7H-pyrimido[4,5-b]azepine-6-carboxamide ditrifluoroacetate

Using 0.1 M solution of 4-({3-chloro-4-[3-(trifluoromethyl)phenoxy]phenyl}amino)-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of 4-(2-aminoethyl)pyridine in N,N-dimethylformamide (0.5 mL), a similar reaction as in Example 4 was carried out to give the title compound (36.1 mg).

LC-MS found 581 (M+H)⁺ (exact MS calcd for C₂₉H₂₄ClF₃N₆O₂ 580).

### Example 26

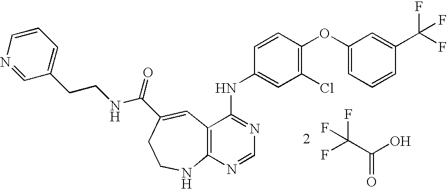

### Production of 4-({3-chloro-4-[3-(trifluoromethyl)phenoxy]phenyl}amino)-N-(2-pyridin-3-ylethyl)-8,9-dihydro-7H-pyrimido[4,5-b]azepine-6-carboxamide ditrifluoroacetate

Using 0.1 M solution of 4-({3-chloro-4-[3-(trifluoromethyl)phenoxy]phenyl}amino)-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of 3-(2-aminoethyl)pyridine in N,N-dimethylformamide (0.5 mL), a similar reaction as in Example 4 was carried out to give the title compound (35.9 mg).

LC-MS found 581 (M+H)⁺ (exact MS calcd for C₂₉H₂₄ClF₃N₆O₂ 580).

### Example 27

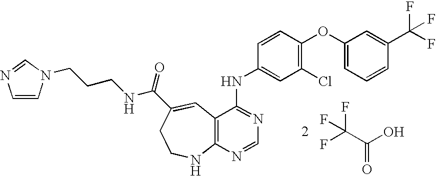

### Production of 4-({3-chloro-4-[3-(trifluoromethyl)phenoxy]phenyl}amino)-N-[3-(1H-imidazol-1-yl) propyl]-8,9-dihydro-7H-pyrimido[4,5-b]azepine-6-carboxamide ditrifluoroacetate

Using 0.1 M solution of 4-({3-chloro-4-[3-(trifluoromethyl)phenoxy]phenyl}amino)-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of 3-(1H-imidazol-1-yl)-1-propylamine in N,N-dimethylformamide (0.5 mL), a similar reaction as in Example 4 was carried out to give the title compound (30.7 mg).

LC-MS found 584 (M+H)⁺ (exact MS calcd for C₂₈H₂₅ClF₃N₇O₂ 583).

### Example 28

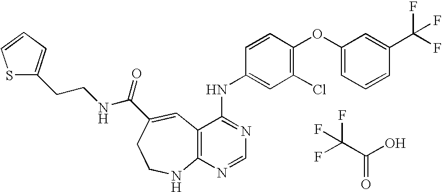

### Production of 4-({3-chloro-4-[3-(trifluoromethyl)phenoxy]phenyl}amino)-N-[2-(2-thienyl)ethyl]-8,9-dihydro-7H-pyrimido[4,5-b]azepine-6-carboxamide trifluoroacetate

Using 0.1 M solution of 4-({3-chloro-4-[3-(trifluoromethyl)phenoxy]phenyl}amino)-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of 2-(2-thienyl)ethylamine in N,N-dimethylformamide (0.5 mL), a similar reaction as in Example 4 was carried out to give the title compound (25.4 mg).

LC-MS found 586 (M+H)⁺ (exact MS calcd for C₂₈H₂₃ClF₃N₅O₂S 585).

### Example 29

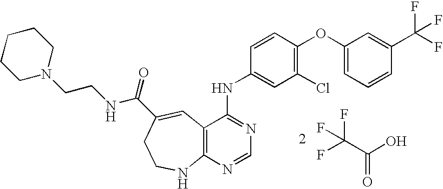

### Production of 4-({3-chloro-4-[3-(trifluoromethyl)phenoxy]phenyl}amino)-N-(2-piperidin-1-ylethyl)-8,9-dihydro-7H-pyrimido[4,5-b]azepine-6-carboxamide ditrifluoroacetate

Using 0.1 M solution of 4-({3-chloro-4-[3-(trifluoromethyl)phenoxy]phenyl}amino)-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of 1-(2-aminoethyl)piperidine in N,N-dimethylformamide (0.5 mL), a similar reaction as in Example 4 was carried out to give the title compound (7.8 mg).

LC-MS found 587 (M+H)⁺ (exact MS calcd for C₂₉H₃₀ClF₃N₆O₂ 586).

### Example 30

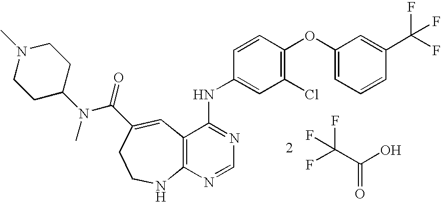

### Production of 4-({3-chloro-4-[3-(trifluoromethyl)phenoxy]phenyl}amino)-N-methyl-N-(1-methylpiperidin-4-yl)-8,9-dihydro-7H-pyrimido[4,5-b]azepine-6-carboxamide ditrifluoroacetate

Using 0.1 M solution of 4-({3-chloro-4-[3-(trifluoromethyl)phenoxy]phenyl}amino)-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of 1-methyl-4-(aminomethyl)piperidin-4-amine in N,N-dimethylformamide (0.5 mL), a similar reaction as in Example 4 was carried out to give the title compound (34.2 mg).

LC-MS found 587 (M+H)⁺ (exact MS calcd for C₂₉H₃₀ClF₃N₆O₂ 586).

### Example 31

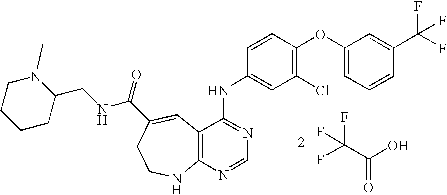

### Production of 4-({3-chloro-4-[3-(trifluoromethyl)phenoxy]phenyl}amino)-N-[(1-methylpiperidin-2-yl)methyl]-8,9-dihydro-7H-pyrimido[4,5-b]azepine-6-carboxamide ditrifluoroacetate

Using 0.1 M solution of 4-({3-chloro-4-[3-(trifluoromethyl)phenoxy]phenyl}amino)-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of (1-methylpiperidin-2-yl)methaneamine in N,N-dimethylformamide (0.5 mL), a similar reaction as in Example 4 was carried out to give the title compound (36.8 mg).

LC-MS found 587 (M+H)⁺ (exact MS calcd for C₂₈H₂₃ClF₃N₅O₂S 586).

### Example 32

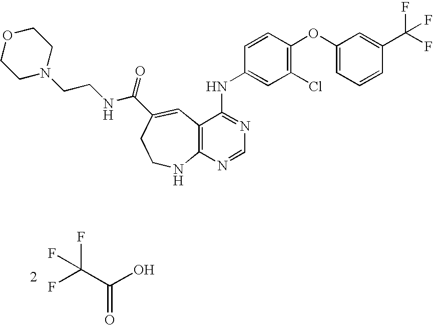

### Production of 4-({3-chloro-4-[3-(trifluoromethyl)phenoxy]phenyl}amino)-N-(2-morpholin-4-ylethyl)-8,9-dihydro-7H-pyrimido[4,5-b]azepine-6-carboxamide ditrifluoroacetate.

Using 0.1 M solution of 4-({3-chloro-4-[3-(trifluoromethyl)phenoxy]phenyl}amino)-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of 4-(2-aminoethyl)morpholine in N,N-dimethylformamide (0.5 mL), a similar reaction as in Example 4 was carried out to give the title compound (34.6 mg).

LC-MS found 589 (M+H)⁺ (exact MS calcd for C₂₈H₂₈ClF₃N₆O₃ 588).

### Example 33

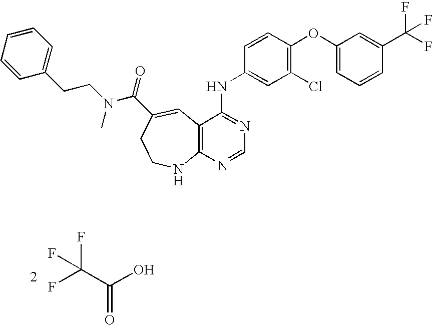

### Production of 4-({3-chloro-4-[3-(trifluoromethyl)phenoxy]phenyl}amino)-N-methyl-N-(2-pyridin-2-ylethyl)-8,9-dihydro-7H-pyrimido[4,5-b]azepine-6-carboxamide ditrifluoroacetate

Using 0.1 M solution of 4-({3-chloro-4-[3-(trifluoromethyl)phenoxy]phenyl}amino)-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of 2-[2-(methylamino)ethyl]pyridine in N,N-dimethylformamide (0.5 mL), a similar reaction as in Example 4 was carried out to give the title compound (32.0 mg).

LC-MS found 595 (M+H)⁺ (exact MS calcd for C₃₀H₂₆ClF₃N₆O₂ 594).

### Example 34

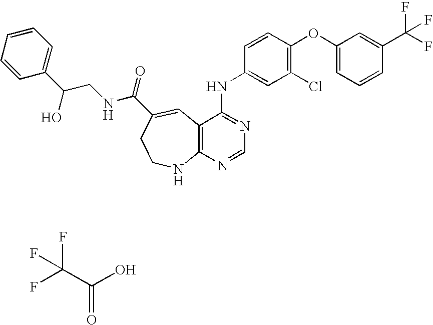

### Production of 4-({3-chloro-4-[3-(trifluoromethyl)phenoxy]phenyl}amino)-N-(2-hydroxy-2-phenylethyl)-8,9-dihydro-7H-pyrimido[4,5-b]azepine-6-carboxamide trifluoroacetate

Using 0.1 M solution of 4-({3-chloro-4-[3-(trifluoromethyl)phenoxy]phenyl}amino)-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of 2-amino-1-phenylethanol in N,N-dimethylformamide (0.5 mL), a similar reaction as in Example 4 was carried out to give the title compound (26.1 mg).

LC-MS found 596 (M+H)⁺ (exact MS calcd for C₃₀H₂₅ClF₃N₅O₃ 595).

### Example 35

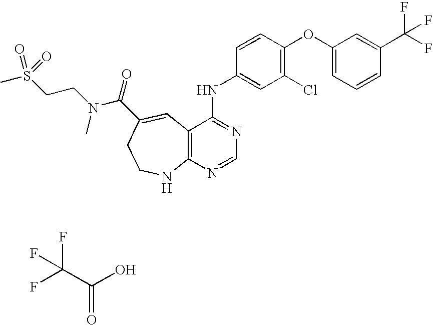

### Production of 4-({3-chloro-4-[3-(trifluoromethyl)phenoxy]phenyl}amino)-N-methyl-N-[2-(methylsulfonyl)ethyl]-8,9-dihydro-7H-pyrimido[4,5-b]azepine-6-carboxamide trifluoroacetate

Using 0.1 M solution of 4-({3-chloro-4-[3-(trifluoromethyl)phenoxy]phenyl}amino)-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of N-methyl-2-(methylsulfonyl)ethaneamine in N,N-dimethylformamide (0.5 mL), a similar reaction as in Example 4 was carried out to give the title compound (28.2 mg).

LC-MS found 596 (M+H)⁺ (exact MS calcd for C₂₆H₂₅ClF₃N₅O₄S 595).

### Example 36

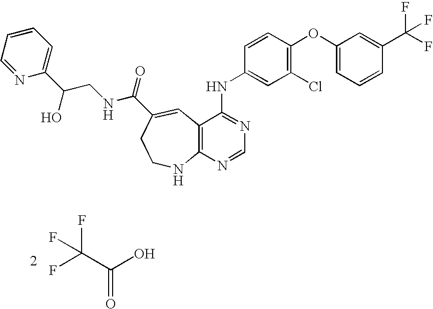

### Production of 4-({3-chloro-4-[3-(trifluoromethyl)phenoxy]phenyl}amino)-N-(2-hydroxy-2-pyridin-2-ylethyl)-8,9-dihydro-7H-pyrimido[4,5-b]azepine-6-carboxamide ditrifluoroacetate

Using 0.1 M solution of 4-({3-chloro-4-[3-(trifluoromethyl)phenoxy]phenyl}amino)-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of 2-amino-1-(pyridin-2-yl)ethanol in N,N-dimethylformamide (0.5 mL), a similar reaction as in Example 4 was carried out to give the title compound (27.6 mg).

LC-MS found 597 (M+H)⁺ (exact MS calcd for C₂₉H₂₄ClF₃N₆O₃ 596).

### Example 37

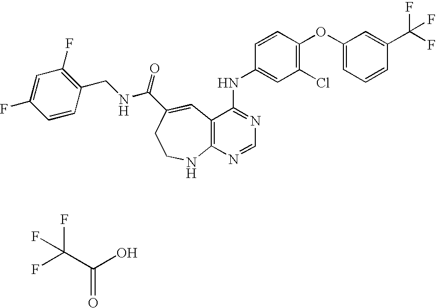

### Production of 4-({3-chloro-4-[3-(trifluoromethyl)phenoxy]phenyl}amino)-N-(2,4-difluorobenzyl)-8,9-dihydro-7H-pyrimido[4,5-b]azepine-6-carboxamide trifluoroacetate

Using 0.1 M solution of 4-({3-chloro-4-[3-(trifluoromethyl)phenoxy]phenyl}amino)-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of 2,4-difluorobenzylamine in N,N-dimethylformamide (0.5 mL), a similar reaction as in Example 4 was carried out to give the title compound (26.9 mg).

LC-MS found 602 (M+H)⁺ (exact MS calcd for C₂₉H₂₁ClF₅N₅O₂ 601).

### Example 38

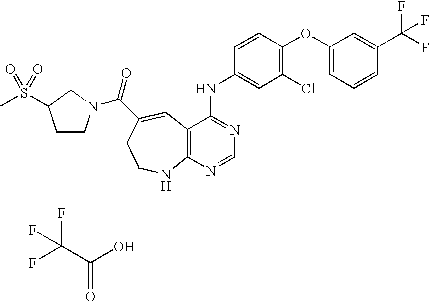

### Production of N-{3-chloro-4-[3-(trifluoromethyl)phenoxy]phenyl}-6-{[3-(methylsulfonyl)pyrrolidin-1-yl]carbonyl}-8,9-dihydro-7H-pyrimido[4,5-b]azepin-4-amine trifluoroacetate

Using 0.1 M solution of 4-({3-chloro-4-[3-(trifluoromethyl)phenoxy]phenyl}amino)-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of 3-(methylsulfonyl)pyrrolidine in N,N-dimethylformamide (0.5 mL), a similar reaction as in Example 4 was carried out to give the title compound (29.8 mg).

LC-MS found 608 (M+H)⁺ (exact MS calcd for C₂₇H₂₅ClF₃N₅O₄S 607).

### Example 39

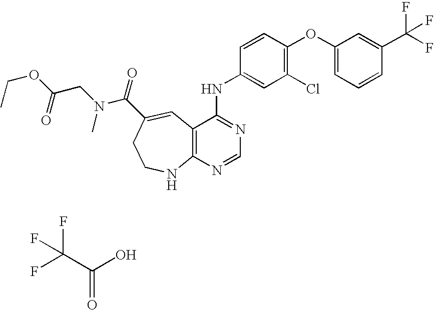

### Production of ethyl N-{[4-({3-chloro-4-[3-(trifluoromethyl)phenoxy]phenyl}amino)-8,9-dihydro-7H-pyrimido[4,5-b]azepin-6-yl]carbonyl}-N-methylglycinate trifluoroacetate

Using 0.1 M solution of 4-({3-chloro-4-[3-(trifluoromethyl)phenoxy]phenyl}amino)-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL), 0.15 M solution of ethyl N-methylglycinate hydrochloride in N,N-dimethylformamide (0.5 mL) and triethylamine (0.01 mL), a similar reaction as in Example 7 was carried out to give the title compound (26.5 mg).

LC-MS found 576 (M+H)⁺ (exact MS calcd for C₂₇H₂₅ClF₃N₅O₄ 575).

### Example 40

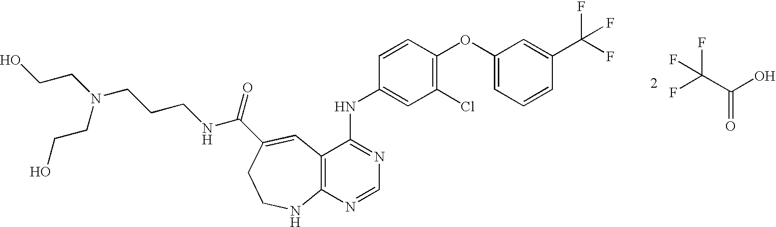

### Production of N-{3-[bis(2-hydroxyethyl)amino]propyl}-4-({3-chloro-4-[3-(trifluoromethyl)phenoxy]phenyl}amino)-8,9-dihydro-7H-pyrimido[4,5-b]azepine-6-carboxamide ditrifluoroacetate

Using 0.1 M solution of 4-({3-chloro-4-[3-(trifluoromethyl)phenoxy]phenyl}amino)-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of 2,2′-[(3-aminopropyl)imino]diethanol in N,N-dimethylformamide (0.5 mL), a similar reaction as in Example 4 was carried out to give the title compound (29.7 mg).

LC-MS found-621 (M+H)⁺ (exact MS calcd for C₂₉H₃₂ClF₃N₆O₄ 620).

### Example 41

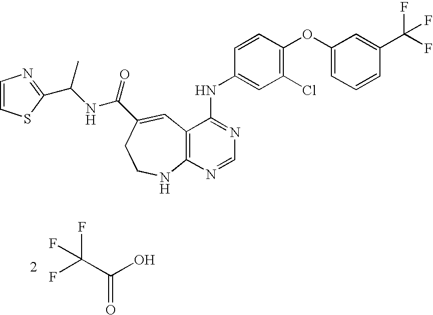

### Production of 4-({3-chloro-4-[3-(trifluoromethyl)phenoxy]phenyl}amino)-N-[1-(1,3-thiazol-2-yl)ethyl]-8,9-dihydro-7H-pyrimido[4,5-b]azepine-6-carboxamide ditrifluoroacetate

Using 0.1 M solution of 4-({3-chloro-4-[3-(trifluoromethyl)phenoxy]phenyl}amino)-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL), 0.15 M solution of 1-(1,3-thiazol-2-yl)ethylamine hydrochloride in N,N-dimethylformamide (0.5 mL) and triethylamine (0.01 mL), a similar reaction as in Example 7 was carried out to give the title compound (19.6 mg).

LC-MS found 587 (M+H)⁺ (exact MS calcd for C₂₇H₂₂ClF₃N₆O₂S 586).

### Example 42

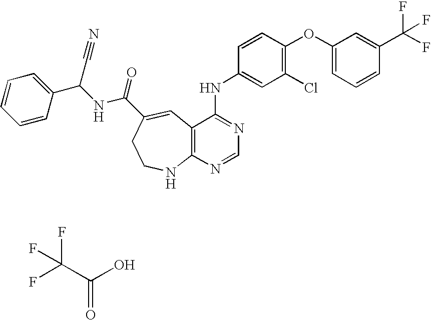

### Production of 4-({3-chloro-4-[3-(trifluoromethyl)phenoxy]phenyl}amino)-N-[cyano(phenyl)methyl]-8,9-dihydro-7H-pyrimido[4,5-b]azepine-6-carboxamide trifluoroacetate

Using 0.1 M solution of 4-({3-chloro-4-[3-(trifluoromethyl)phenoxy]phenyl}amino)-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of amino(phenyl)acetonitrile hydrochloride in N,N-dimethylformamide (0.5 mL) and triethylamine (0.01 mL), a similar reaction as in Example 7 was carried out to give the title compound (23.5 mg).

LC-MS found 591 (M+H)⁺ (exact MS calcd for C₃₀H₂₂ClF₃N₆O₂ 590).

### Example 43

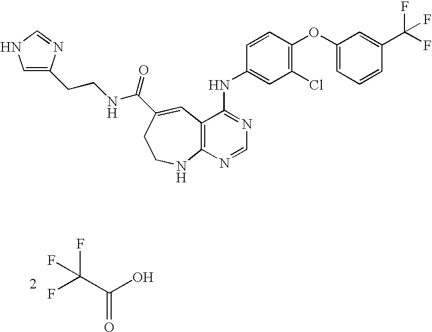

### Production of 4-({3-chloro-4-[3-(trifluoromethyl)phenoxy]phenyl}amino)-N-[2-(1H-imidazol-4-yl)ethyl]-8,9-dihydro-7H-pyrimido[4,5-b]azepine-6-carboxamide ditrifluoroacetate

Using 0.1 M solution of 4-({3-chloro-4-[3-(trifluoromethyl)phenoxy]phenyl}amino)-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL), 0.15 M solution of 2-(1H-imidazol-4-yl)ethylamine dihydrochloride in N,N-dimethylformamide (0.5 mL) and triethylamine (0.02 mL), a similar reaction as in Example 7 was carried out to give the title compound (25.1 mg).

LC-MS found 570 (M+H)⁺ (exact MS calcd for C₂₇H₂₃ClF₃N₇O₂ 569).

### Example 44

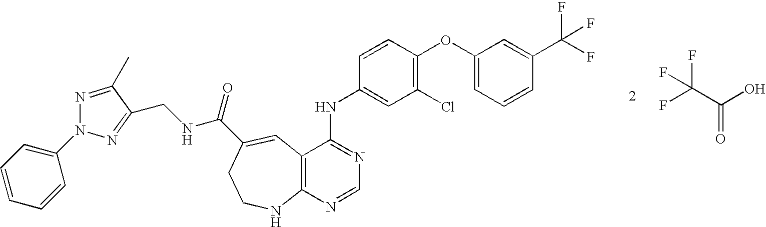

### Production of 4-({3-chloro-4-[3-(trifluoromethyl)phenoxy]phenyl}amino)-N-[(5-methyl-2-phenyl-2H-1,2,3-triazol-4-yl)methyl]-8,9-dihydro-7H-pyrimido[4,5-b]azepine-6-carboxamide ditrifluoroacetate

Using 0.1 M solution of 4-({3-chloro-4-[3-(trifluoromethyl)phenoxy]phenyl}amino)-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of 1-(5-methyl-2-phenyl-2H-1,2,3-triazol-4-yl)methylamine in N,N-dimethylformamide (0.5 mL), a similar reaction as in Example 4 was carried out to give the title compound (29.3 mg).

LC-MS found 647 (M+H)⁺ (exact MS calcd for C₃₂H₂₆ClF₃N₈O₂ 646).

### Example 45

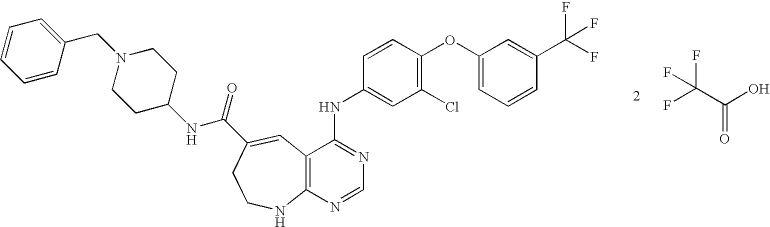

### Production of N-(1-benzylpiperidin-4-yl)-4-({3-chloro-4-[3-(trifluoromethyl)phenoxy]phenyl}amino)-8,9-dihydro-7H-pyrimido[4,5-b]azepine-6-carboxamide ditrifluoroacetate

Using 0.1 M solution of 4-({3-chloro-4-[3-(trifluoromethyl)phenoxy]phenyl}amino)-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of 4-amino-1-benzylpiperidine in N,N-dimethylformamide (0.5 mL), a similar reaction as in Example 4 was carried out to give the title compound (35.7 mg).

LC-MS found 649 (M+H)⁺ (exact MS calcd for C₃₄H₃₂ClF₃N₆O₂ 648).

### Example 46

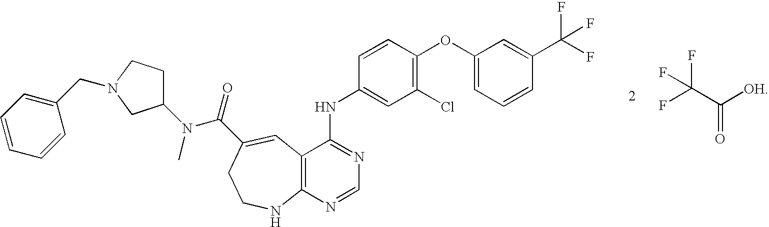

### Production of N-(1-benzylpyrrolidin-3-yl)-4-({3-chloro-4-[3-(trifluoromethyl)phenoxy]phenyl}amino)-N-methyl-8,9-dihydro-7H-pyrimido[4,5-b]azepine-6-carboxamide ditrifluoroacetate

Using 0.1 M solution of 4-({3-chloro-4-[3-(trifluoromethyl)phenoxy]phenyl}amino)-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of 1-benzyl-3-(methylamino)pyrrolidine in N,N-dimethylformamide (0.5 mL), a similar reaction as in Example 4 was carried out to give the title compound (32.7 mg).

LC-MS found 649 (M+H)⁺ (exact MS Calcd for C₃₄H₃₂ClF₃N₆O₂ 648).

### Example 47

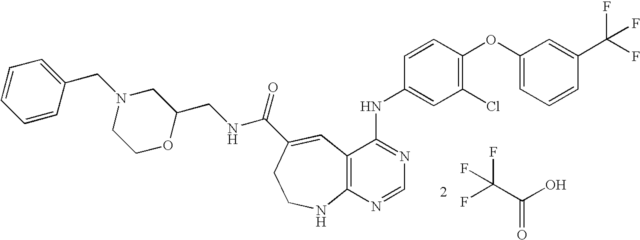

### Production of N-[(4-benzylmorpholin-2-yl)methyl]-4-({3-chloro-4-[3-(trifluoromethyl)phenoxy]phenyl}amino)-8,9-dihydro-7H-pyrimido[4,5-b]azepine-6-carboxamide ditrifluoroacetate

Using 0.1 M solution of 4-({3-chloro-4-[3-(trifluoromethyl)phenoxy]phenyl}amino)-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of 1-(4-benzylmorpholin-2-yl)methylamine in N,N-dimethylformamide (0.5 mL), a similar reaction as in Example 4 was carried out to give the title compound (42.6 mg).

LC-MS found 665 (M+H)⁺ (exact MS calcd for C₃₄H₃₂ClF₃N₆O₃ 664).

### Example 48

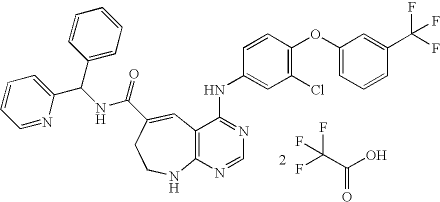

### Production of 4-({3-chloro-4-[3-(trifluoromethyl)phenoxy]phenyl}amino)-N-[phenyl(pyridin-2-yl)methyl]-8,9-dihydro-7H-pyrimido[4,5-b]azepine-6-carboxamide ditrifluoroacetate

Using 0.1 M solution of 4-({3-chloro-4-[3-(trifluoromethyl)phenoxy]phenyl}amino)-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL), 0.15 M solution of 1-phenyl-1-pyridin-2-ylmethylamine hydrochloride in N,N-dimethylformamide (0.5 mL) and triethylamine (0.01 mL), a similar reaction as in Example 7 was carried out to give the title compound (35.4 mg).

LC-MS found 643 (M+H)⁺ (exact MS calcd for C₃₄H₂₆ClF₃N₆O₂ 642).

### Example 49

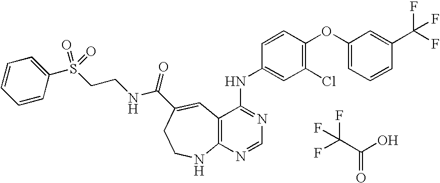

### Production of 4-({3-chloro-4-[3-(trifluoromethyl)phenoxy]phenyl}amino)-N-[2-(phenylsulfonyl)ethyl]-8,9-dihydro-7H-pyrimido[4,5-b]azepine-6-carboxamide trifluoroacetate

Using 0.1 M solution of 4-({3-chloro-4-[3-(trifluoromethyl)phenoxy]phenyl}amino)-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL), 0.15 M solution of 2-(phenylsulfonyl)ethylamine hydrochloride in N,N-dimethylformamide (0.5 mL) and triethylamine (0.01 mL), a similar reaction as in Example 7 was carried out to give the title compound (23.4 mg).

LC-MS found 644 (M+H)⁺ (exact MS calcd for C₃₀H₂₅ClF₃N₅O₄S 643).

### Example 50

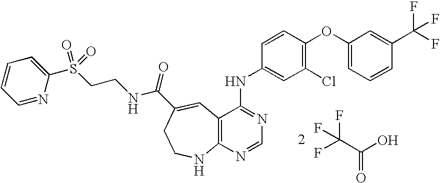

### Production of 4-({3-chloro-4-[3-(trifluoromethyl)phenoxy]phenyl}amino)-N-[2-(pyridin-2-ylsulfonyl)ethyl]-8,9-dihydro-7H-pyrimido[4,5-b]azepine-6-carboxamide ditrifluoroacetate

Using 0.1 M solution of 4-({3-chloro-4-[3-(trifluoromethyl)phenoxy]phenyl}amino)-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL), 0.15 M solution of 2-[(pyridin-2-yl)sulfonyl]ethylamine dihydrochloride in N,N-dimethylformamide (0.5 mL) and triethylamine (0.01 mL), a similar reaction as in Example 7 was carried out to give the title compound (30.5 mg).

LC-MS found 645 (M+H)⁺ (exact MS calcd for C₂₉H₂₄ClF₃N₆O₄S 644).

### Example 51

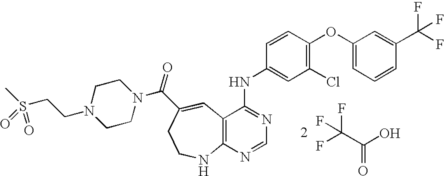

### Production of N-{3-chloro-4-[3-(trifluoromethyl)phenoxy]phenyl}-6-({4-[2-(methylsulfonyl)ethyl]piperazin-1-yl}carbonyl)-8,9-dihydro-7H-pyrimido[4,5-b]azepin-4-amine ditrifluoroacetate

Using 0.1 M solution of 4-({3-chloro-4-[3-(trifluoromethyl)phenoxy]phenyl}amino)-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL), 0.15 M solution of 1-[2-(methylsulfonyl)ethyl]piperazine dihydrochloride in N,N-dimethylformamide (0.5 mL) and triethylamine (0.01 mL), a similar reaction as in Example 7 was carried out to give the title compound (30.4 mg).

LC-MS found 651 (M+H)⁺ (exact MS calcd for C₂₉H₃₀ClF₃N₆O₄S 650).

### Example 52

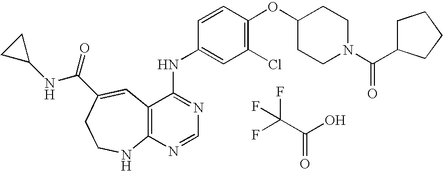

### Production of 4-[(3-chloro-4-{[1-(cyclopentylcarbonyl)piperidin-4-yl]oxy}phenyl)amino]-N-cyclopropyl-8,9-dihydro-7H-pyrimido[4,5-b]azepine-6-carboxamide trifluoroacetate

### (i) Production of 4-[(3-chloro-4-{[1-(cyclopentylcarbonyl)piperidin-4-yl]oxy}phenyl)amino]-8,9-dihydro-7H-pyrimido[4,5-b]azepine-6-carboxylic acid

Methyl 4-[(3-chloro-4-{[1-(cyclopentylcarbonyl)piperidin-4-yl]oxy}phenyl)amino]-8,9-dihydro-7H-pyrimido[4,5-b]azepine-6-carboxylate (1.83 g) was dissolved in a mixed solvent of tetrahydrofuran (60 mL)/ethanol (60 mL). 1N aqueous sodium hydroxide solution (10 mL) was added to the mixture and stirred for 5 hr at room temperature. IN hydrochloric acid was added to the reaction mixture, the pH was adjusted to 3 to 4, and the mixture was diluted with water. The precipitate was collected by filtration, washed with diethyl ether and dried to give the title compound (1.23 g) as a pale-yellow powder.

¹H-NMR (DMSO-d₆) δ: 1.40-2.05 (12H, m), 2.62-2.78 (2H, m), 2.90-3.08 (1H, m), 3.24-3.53 (4H, m), 3.64-3.82 (2H, m), 4.52-4.73 (1H, m), 7.17 (1H, d, J=9.0 Hz), 7.38 (1H, dd, J=9.0 Hz, 2.5 Hz), 7.61 (1H, d, J=2.5 Hz), 7.68 (1H, s), 7.79 (1H, t, J=4.9 Hz), 7.91 (1H, s), 9.16 (1H, s), 12.23 (1H, s).

### (ii) Production of 4-[(3-chloro-4-{[1-(cyclopentylcarbonyl)piperidin-4-yl]oxy}phenyl)amino]-N-cyclopropyl-8,9-dihydro-7H-pyrimido[4,5-b]azepine-6-carboxamide trifluoroacetate

Using 0.1 M solution of 4-[(3-chloro-4-{[1-(cyclopentylcarbonyl)piperidin-4-yl]oxy}phenyl)amino]-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of cyclopropylamine in N,N-dimethylformamide (0.5 mL), a similar reaction as in Example 4 was carried out to give the title compound (27.0 mg).

LC-MS found 551 (M+H)⁺ (exact MS calcd for C₂₉H₃₅ClN₆O₃ 550).

### Example 53

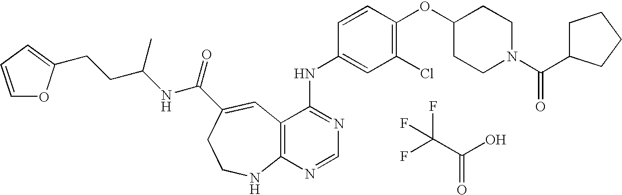

### Production of 4-[(3-chloro-4-{[1-(cyclopentylcarbonyl)piperidin-4-yl]oxy}phenyl)amino]-N-[3-(2-furyl)-1-methylpropyl]-8,9-dihydro-7H-pyrimido[4,5-b]azepine-6-carboxamide trifluoroacetate

Using 0.1 M solution of 4-[(3-chloro-4-{[1-(cyclopentylcarbonyl)piperidin-4-yl]oxy}phenyl)amino]-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of 4-(2-furyl)butane-2-amine in N,N-dimethylformamide (0.5 mL), a similar reaction as in Example 4 was carried out to give the title compound (28.4 mg).

LC-MS found 633 (M+H)⁺ (exact MS calcd for C₃₄H₄₁ClN₆O₄ 632).

### Example 54

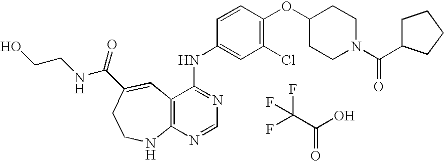

### Production of 4-[(3-chloro-4-{[1-(cyclopentylcarbonyl)piperidin-4-yl]oxy}phenyl)amino]-N-(2-hydroxyethyl)-8,9-dihydro-7H-pyrimido[4,5-b]azepine-6-carboxamide trifluoroacetate

Using 0.1 M solution of 4-[(3-chloro-4-{[1-(cyclopentylcarbonyl)piperidin-4-yl]oxy}phenyl)amino]-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochlorides 1/1 in N,N-dimethylformamide (0.5 mL) and 0.15 M solution of 2-aminoethanol in N,N-dimethylformamide (0.5 mL), a similar reaction as in Example 4 was carried out to give the title compound (28.1 mg).

LC-MS found 555 (M+H)⁺ (exact MS calcd for C₂₈H₃₅ClN₆O₄ 554).

### Example 55

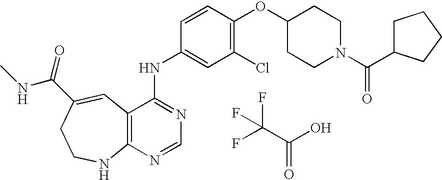

### Production of 4-[(3-chloro-4-{[1-(cyclopentylcarbonyl)piperidin-4-yl]oxy}phenyl)amino]-N-methyl-8,9-dihydro-7H-pyrimido[4,5-b]azepine-6-carboxamide trifluoroacetate

Using 0.1 M solution of 4-[(3-chloro-4-{[1-(cyclopentylcarbonyl)piperidin-4-yl]oxy}phenyl)amino]-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL), 0.15 M solution of methylamine hydrochloride in N,N-dimethylformamide (0.5 mL) and triethylamine (0.01 mL), a similar reaction as in Example 7 was carried out to give the title compound (27.2 mg).

LC-MS found 525 (M+H)⁺ (exact MS calcd for C₂₇H₃₃ClN₆O₃ 524).

### Example 56

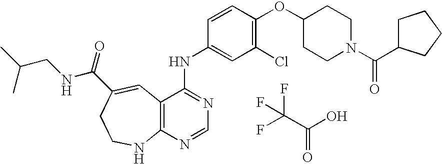

### Production of 4-[(3-chloro-4-{[1-(cyclopentylcarbonyl)piperidin-4-yl]oxy}phenyl)amino]-N-isobutyl-8,9-dihydro-7H-pyrimido[4,5-b]azepine-6-carboxamide trifluoroacetate

Using 0.1 M solution of 4-[(3-chloro-4-{[1-(cyclopentylcarbonyl)piperidin-4-yl]oxy}phenyl)amino]-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of isobutylamine in N,N-dimethylformamide (0.5 mL), a similar reaction as in Example 4 was carried out to give the title compound (28.6 mg).

LC-MS found 567 (M+H)⁺ (exact MS calcd for C₃₀H₃₉ClN₆O₃ 566).

### Example 57

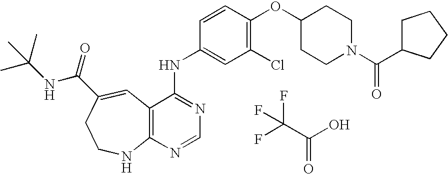

### Production of N-(tert-butyl)-4-[(3-chloro-4-{[1-(cyclopentylcarbonyl)piperidin-4-yl]oxy}phenyl)amino]-8,9-dihydro-7H-pyrimido[4,5-b]azepine-6-carboxamide trifluoroacetate

Using 0.1 M solution of 4-[(3-chloro-4-{[1-(cyclopentylcarbonyl)piperidin-4-yl]oxy}phenyl)amino]-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of tert-butylamine in N,N-dimethylformamide (0.5 mL), a similar reaction as in Example 4 was carried out to give the title compound (22.1 mg).

LC-MS found 567 (M+H)⁺ (exact MS calcd for C₃₀H₃₉ClN₆O₃ 566).

### Example 58

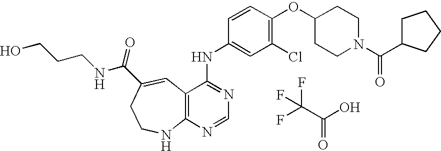

### Production of 4-[(3-chloro-4-{[1-(cyclopentylcarbonyl)piperidin-4-yl]oxy}phenyl)amino]-N-(3-hydroxypropyl)-8,9-dihydro-7H-pyrimido[4,5-b]azepine-6-carboxamide trifluoroacetate

Using 0.1 M solution of 4-[(3-chloro-4-{[1-(cyclopentylcarbonyl)piperidin-4-yl]oxy}phenyl)amino]-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of 3-amino-1-propanol in N,N-dimethylformamide (0.5 mL), a similar reaction as in Example 4 was carried out to give the title compound (23.8 mg).

LC-MS found 569 (M+H)⁺ (exact MS calcd for C₃₀H₃₉ClN₆O₃ 568).

### Example 59

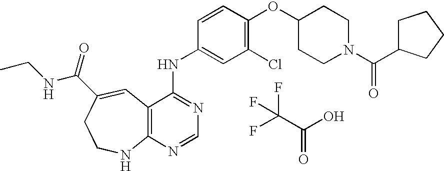

### Production of 4-[(3-chloro-4-{[1-(cyclopentylcarbonyl)piperidin-4-yl]oxy}phenyl)amino]-N-ethyl-8,9-dihydro-7H-pyrimido[4,5-b]azepine-6-carboxamide trifluoroacetate

Using 0.1 M solution of 4-[(3-chloro-4-{[1-(cyclopentylcarbonyl)piperidin-4-yl]oxy}phenyl)amino]-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL), 0.15 M solution of ethylamine hydrochloride in N,N-dimethylformamide (0.5 mL) and triethylamine (0.01 mL), a similar reaction as in Example 7 was carried out to give the title compound (26.4 mg).

LC-MS found 539 (M+H)⁺ (exact MS calcd for C₂₈H₃₅ClN₆O₃ 538).

### Example 60

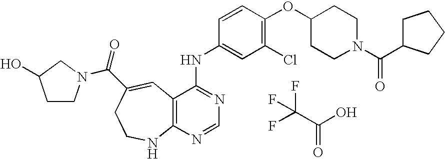

### Production of 1-({4-[(3-chloro-4-{[1-(cyclopentylcarbonyl)piperidin-4-yl]oxy}phenyl)amino]-8,9-dihydro-7H-pyrimido[4,5-b]azepin-6-yl}carbonyl)pyrrolidin-3-ol trifluoroacetate

Using 0.1 M solution of 4-[(3-chloro-4-{[1-(cyclopentylcarbonyl)piperidin-4-yl]oxy}phenyl)amino]-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of pyrrolidin-3-ol in N,N-dimethylformamide (0.5 mL), a similar reaction as in Example 4 was carried out to give the title compound (29.0 mg).

LC-MS found 581 (M+H)⁺ (exact MS calcd for C₃₀H₃₇ClN₆O₄ 580).

### Example 61

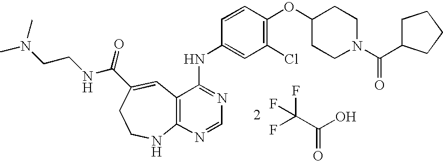

### Production of 4-[(3-chloro-4-{[1-(cyclopentylcarbonyl)piperidin-4-yl]oxy}phenyl)amino]-N-[2-(dimethylamino)ethyl]-8,9-dihydro-7H-pyrimido[4,5-b]azepine-6-carboxamide ditrifluoroacetate

Using 0.1 M solution of 4-[(3-chloro-4-{[1-(cyclopentylcarbonyl)piperidin-4-yl]oxy}phenyl)amino]-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of N,N-dimethylethane-1,2-diamine in N,N-dimethylformamide (0.5 mL), a similar reaction as in Example 4 was carried out to give the title compound (32.9 mg).

LC-MS found 582 (M+H)⁺ (exact MS calcd for C₃₀H₄₀ClN₇O₃ 581).

### Example 62

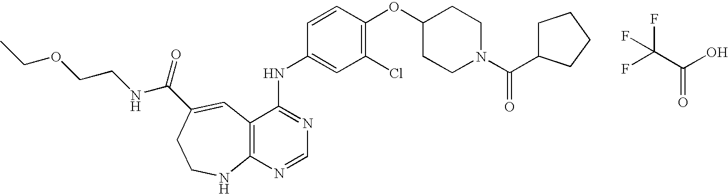

### Production of 4-[(3-chloro-4-{[1-(cyclopentylcarbonyl)piperidin-4-yl]oxy}phenyl)amino]-N-(2-ethoxyethyl)-8,9-dihydro-7H-pyrimido[4,5-b]azepine-6-carboxamide trifluoroacetate

Using 0.1 M solution of 4-[(3-chloro-4-{[1-(cyclopentylcarbonyl)piperidin-4-yl]oxy}phenyl)amino]-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of 2-ethoxyethylamine in N,N-dimethylformamide (0.5 mL), a similar reaction as in Example 4 was carried out to give the title compound (28.7 mg).

LC-MS found 583 (M+H)⁺ (exact MS calcd for C₃₀H₃₉ClN₆O₄ 582).

### Example 63

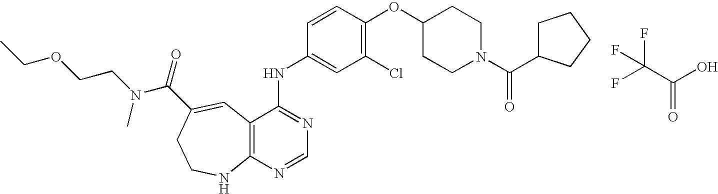

### Production of 4-[(3-chloro-4-{[1-(cyclopentylcarbonyl)piperidin-4-yl]oxy}phenyl)amino]-N-(2-methoxyethyl)-N-methyl-8,9-dihydro-7H-pyrimido[4,5-b]azepine-6-carboxamide trifluoroacetate

Using 0.1 M solution of 4-[(3-chloro-4-{[1-(cyclopentylcarbonyl)piperidin-4-yl]oxy}phenyl)amino]-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of N-(2-methoxyethyl)methylamine in N,N-dimethylformamide (0.5 mL), a similar reaction as in Example 4 was carried out to give the title compound (28.5 mg).

LC-MS found 583 (M+H)⁺ (exact MS calcd for C₃₀H₃₉ClN₆O₄ 582).

### Example 64

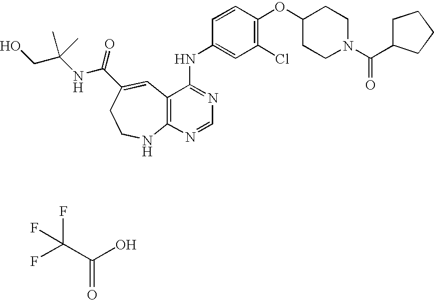

### Production of 4-[(3-chloro-4-{[1-(cyclopentylcarbonyl)piperidin-4-yl]oxy}phenyl)amino]-N-(2-hydroxy-1,1-dimethylethyl)-8,9-dihydro-7H-pyrimido[4,5-b]azepine-6-carboxamide trifluoroacetate.

Using 0.1 M solution of 4-[(3-chloro-4-{[1-(cyclopentylcarbonyl)piperidin-4-yl]oxy}phenyl)amino]-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of 2-amino-2-methyl-1-propanol in N,N-dimethylformamide (0.5 mL), a similar reaction as in Example 4 was carried out to give the title compound (16.9 mg).

LC-MS found 583 (M+H)⁺ (exact MS calcd for C₃₀H₃₉ClN₆O₄ 582).

### Example 65

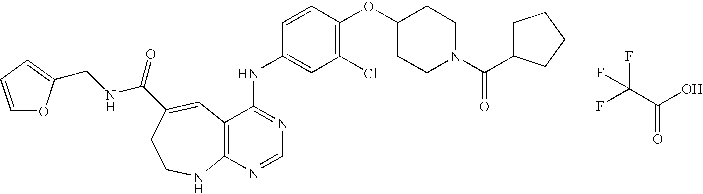

### Production of 4-[(3-chloro-4-{[1-(cyclopentylcarbonyl)piperidin-4-yl]oxy}phenyl)amino]-N-(2-furylmethyl)-8,9-dihydro-7H-pyrimido[4,5-b]azepine-6-carboxamide trifluoroacetate

Using 0.1 M solution of 4-[(3-chloro-4-{[1-(cyclopentylcarbonyl)piperidin-4-yl]oxy}phenyl)amino]-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of furfurylamine in N,N-dimethylformamide (0.5 mL), a similar reaction as in Example 4 was carried out to give the title compound (28.9 mg).

LC-MS found 591 (M+H)⁺ (exact MS calcd for C₃₁H₃₅ClN₆O₄ 590).

### Example 66

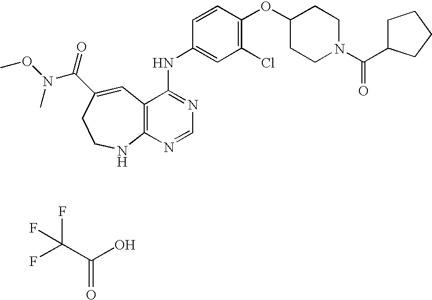

### Production of 4-[(3-chloro-4-{[1-(cyclopentylcarbonyl)piperidin-4-yl]oxy}phenyl)amino]-N-methoxy-N-methyl-8,9-dihydro-7H-pyrimido[4,5-b]azepine-6-carboxamide trifluoroacetate

Using 0.1 M solution of 4-[(3-chloro-4-{[1-(cyclopentylcarbonyl)piperidin-4-yl]oxy}phenyl)amino]-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL), 0.15 M solution of N,O-dimethylhydroxylamine hydrochloride in N,N-dimethylformamide (0.5 mL) and triethylamine (0.01 mL), a similar reaction as in Example 7 was carried out to give the title compound (20.8 mg).

LC-MS found 555 (M+H)⁺ (exact MS calcd for C₂₈H₃₅ClN₆O₄ 554).

### Example 67

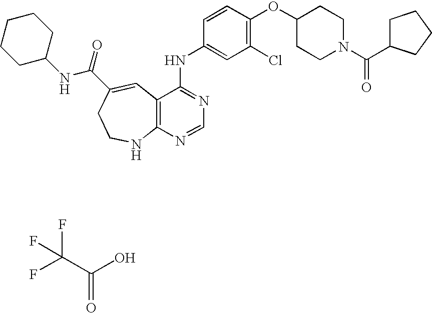

### Production of 4-[(3-chloro-4-{[1-(cyclopentylcarbonyl)piperidin-4-yl]oxy}phenyl)amino]-N-cyclohexyl-8,9-dihydro-7H-pyrimido[4,5-b]azepine-6-carboxamide trifluoroacetate

Using 0.1 M solution of 4-[(3-chloro-4-{[1-(cyclopentylcarbonyl)piperidin-4-yl]oxy}phenyl)amino]-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of cyclohexylamine in N,N-dimethylformamide (0.5 mL), a similar reaction as in Example 4 was carried out to give the title compound (26.3 mg).

LC-MS found 593 (M+H)⁺ (exact MS calcd for C₃₂H₄₁ClN₆O₃ 592).

### Example 68

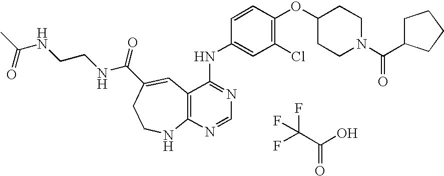

### Production of N-[2-(acetylamino)ethyl]-4-[(3-chloro-4-{[1-(cyclopentylcarbonyl)piperidin-4-yl]oxy}phenyl)amino]-8,9-dihydro-7H-pyrimido[4,5-b]azepine-6-carboxamide trifluoroacetate

Using 0.1 M solution of 4-[(3-chloro-4-{[1-(cyclopentylcarbonyl)piperidin-4-yl]oxy}phenyl)amino]-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of N-(2-aminoethyl)acetamide in N,N-dimethylformamide (0.5 mL), a similar reaction as in Example 4 was carried out to give the title compound (25.1 mg).

LC-MS found 596 (M+H)⁺ (exact MS calcd for C₃₀H₃₈ClN₇O₄ 595).

### Example 69

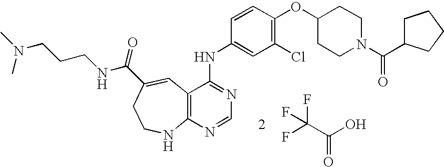

### Production of 4-[(3-chloro-4-{[1-(cyclopentylcarbonyl)piperidin-4-yl]oxy}phenyl)amino]-N-[3-(dimethylamino)propyl]-8,9-dihydro-7H-pyrimido[4,5-b]azepine-6-carboxamide ditrifluoroacetate

Using 0.1 M solution of 4-[(3-chloro-4-{[1-(cyclopentylcarbonyl)piperidin-4-yl]oxy}phenyl)amino]-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of N,N-dimethyl-1,3-propanediamine in N,N-dimethylformamide (0.5 mL), a similar reaction as in Example 4 was carried out to give the title compound (31.5 mg).

LC-MS found 596 (M+H)⁺ (exact MS calcd for C₃₁H₄₂ClN₇O₃ 595).

### Example 70

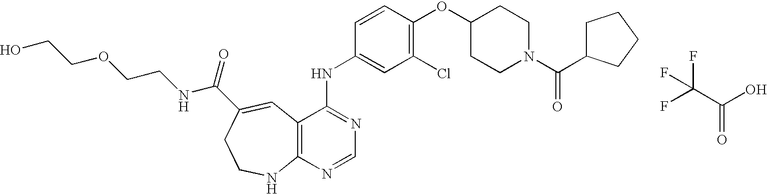

### Production of 4-[(3-chloro-4-{[1-(cyclopentylcarbonyl)piperidin-4-yl]oxy}phenyl)amino]-N-[2-(2-hydroxyethoxy)ethyl]-8,9-dihydro-7H-pyrimido[4,5-b]azepine-6-carboxamide trifluoroacetate

Using 0.1 M solution of 4-[(3-chloro-4-{[1-(cyclopentylcarbonyl)piperidin-4-yl]oxy}phenyl)amino]-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of 2-(2-aminoethoxy)ethanol in N,N-dimethylformamide (0.5 mL), a similar reaction as in Example 4 was carried out to give the title compound (26.1 mg).

LC-MS found 599 (M+H)⁺ (exact MS calcd for C₃₀H₃₉ClN₆O₅ 598).

### Example 71

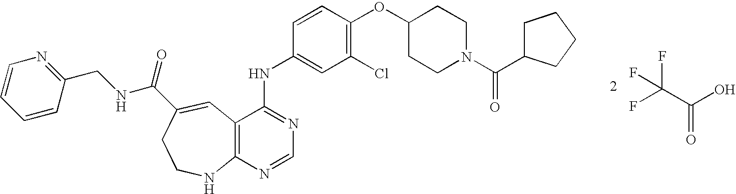

### Production of 4-[(3-chloro-4-{[1-(cyclopentylcarbonyl)piperidin-4-yl]oxy}phenyl)amino]-N-(pyridin-2-ylmethyl)-8,9-dihydro-7H-pyrimido[4,5-b]azepine-6-carboxamide ditrifluoroacetate

Using 0.1 M solution of 4-[(3-chloro-4-{[1-(cyclopentylcarbonyl)piperidin-4-yl]oxy}phenyl)amino]-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of 2-(aminomethyl)pyridine in N,N-dimethylformamide (0.5 mL), a similar reaction as in Example 4 was carried out to give the title compound (33.0 mg).

LC-MS found 602 (M+H)⁺ (exact MS calcd for C₃₂H₃₆ClN₇O₃ 601).

### Example 72

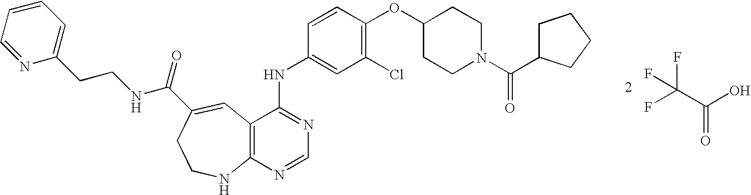

### Production of 4-[(3-chloro-4-{[1-(cyclopentylcarbonyl)piperidin-4-yl]oxy}phenyl)amino]-N-(2-pyridin-2-ylethyl)-8,9-dihydro-7H-pyrimido[4,5-b]azepine-6-carboxamide ditrifluoroacetate

Using 0.1 M solution of 4-[(3-chloro-4-{[1-(cyclopentylcarbonyl)piperidin-4-yl]oxy}phenyl)amino]-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of 2-(2-aminoethyl)pyridine in N,N-dimethylformamide (0.5 mL), a similar reaction as in Example 4 was carried out to give the title compound (34.8 mg).

LC-MS found 616 (M+H)⁺ (exact MS calcd for C₃₃H₃₈ClN₇O₃ 615).

### Example 73

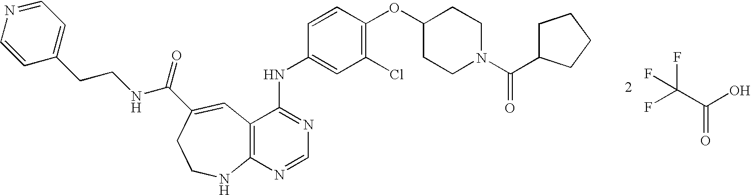

### Production of 4-[(3-chloro-4-{[1-(cyclopentylcarbonyl)piperidin-4-yl]oxy}phenyl)amino]-N-(2-pyridin-4-ylethyl)-8,9-dihydro-7H-pyrimido[4,5-b]azepine-6-carboxamide ditrifluoroacetate

Using 0.1 M solution of 4-[(3-chloro-4-{[1-(cyclopentylcarbonyl)piperidin-4-yl]oxy}phenyl)amino]-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of 4-(2-aminoethyl)pyridine in N,N-dimethylformamide (0.5 mL), a similar reaction, as in Example 4 was carried out to give the title compound (36.2 mg).

LC-MS found 616 (M+H)⁺ (exact MS calcd for C₃₃H₃E₃ClN₇O₃ 615).

### Example 74

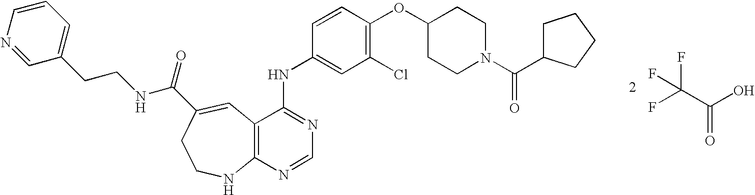

### Production of 4-[(3-chloro-4-{[1-(cyclopentylcarbonyl)piperidin-4-yl]oxy}phenyl)amino]-N-(2-pyridin-3-ylethyl)-8,9-dihydro-7H-pyrimido[4,5-b]azepine-6-carboxamide ditrifluoroacetate

Using 0.1 M solution of 4-[(3-chloro-4-{[1-(cyclopentylcarbonyl)piperidin-4-yl]oxy}phenyl)amino]-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of 3-(2-aminoethyl)pyridine in N,N-dimethylformamide (0.5 mL), a similar reaction as in Example 4 was carried out to give the title compound (35.1 mg).

LC-MS found 616 (M+H)⁺ (exact MS calcd for C₃₃H₃₈ClN₇O₃ 615).

### Example 75

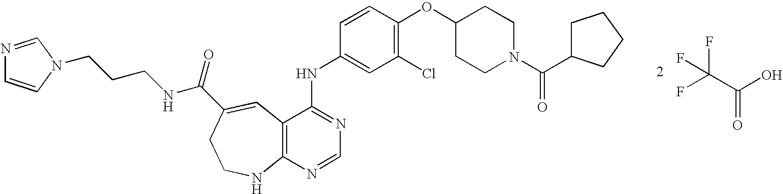

### Production of 4-[(3-chloro-4-{[1-(cyclopentylcarbonyl)piperidin-4-yl]oxy}phenyl)amino]-N-[3-(1H-imidazol-1-yl)propyl]-8,9-dihydro-7H-pyrimido[4,5-b]azepine-6-carboxamide ditrifluoroacetate

Using 0.1 M solution of 4-[(3-chloro-4-{[1-(cyclopentylcarbonyl)piperidin-4-yl]oxy}phenyl)amino]-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of 3-(1H-imidazol-1-yl)-1-propylamine in N,N-dimethylformamide (0.5 mL), a similar reaction as in Example 4 was carried out to give the title compound (32.7 mg).

LC-MS found 619 (M+H)⁺ (exact MS calcd for C₃₂H₃₉ClN₈O₃ 618).

### Example 76

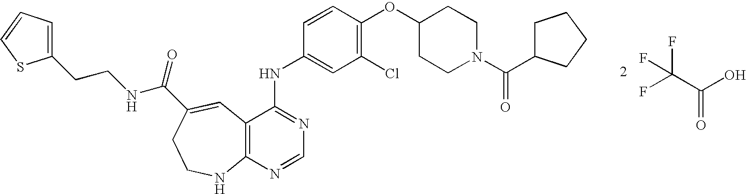

### Production of 4-[(3-chloro-4-{[1-(cyclopentylcarbonyl)piperidin-4-yl]oxy}phenyl)amino]-N-[2-(2-thienyl)ethyl]-8,9-dihydro-7H-pyrimido[4,5-b]azepine-6-carboxamide trifluoroacetate

Using 0.1 M solution of 4-[(3-chloro-4-{[1-(cyclopentylcarbonyl)piperidin-4-yl]oxy}phenyl)amino]-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of 2-(2-thienyl)ethylamine in N,N-dimethylformamide (0.5 mL), a similar reaction as in Example 4 was carried out to give the title compound (28.1 mg).

LC-MS found 621 (M+H)⁺ (exact MS calcd for C₃₂H₃₇ClN₆O₃S 620).

### Example 77

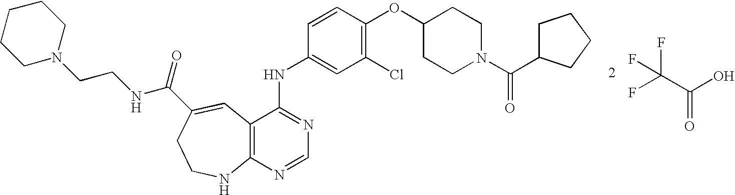

### Production of 4-[(3-chloro-4-{[1-(cyclopentylcarbonyl)piperidin-4-yl]oxy}phenyl)amino]-N-(2-piperidin-1-ylethyl)-8,9-dihydro-7H-pyrimido[4,5-b]azepine-6-carboxamide ditrifluoroacetate

Using 0.1 M solution of 4-[(3-chloro-4-{[1-(cyclopentylcarbonyl)piperidin-4-yl]oxy}phenyl)amino]-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of 1-(2-aminoethyl)piperidine in N,N-dimethylformamide (0.5 mL), a similar reaction as in Example 4 was carried out to give the title compound (35.5 mg).

LC-MS found 622 (M+H)⁺ (exact MS calcd for C₃₃H₄₄ClN₇O₃ 621).

### Example 78

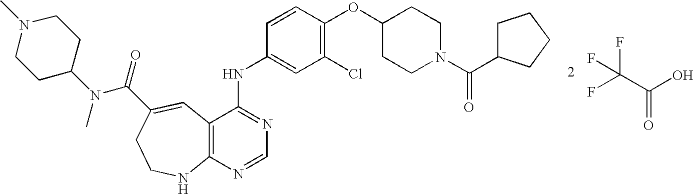

### Production of 4-[(3-chloro-4-{[1-(cyclopentylcarbonyl)piperidin-4-yl]oxy}phenyl)amino]-N-methyl-N-(1-methylpiperidin-4-yl)-8,9-dihydro-7H-pyrimido[4,5-b]azepine-6-carboxamide ditrifluoroacetate

Using 0.1 M solution of 4-[(3-chloro-4-{[1-(cyclopentylcarbonyl)piperidin-4-yl]oxy}phenyl)amino]-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of 1-methyl-4-(aminomethyl)piperidin-4-amine in N,N-dimethylformamide (0.5 mL), a similar reaction as in Example 4 was carried out to give the title compound (35.8 mg).

LC-MS found 622 (M+H)⁺ (exact MS calcd for C₃₃H₄₄ClN₇O₃ 621).

### Example 79

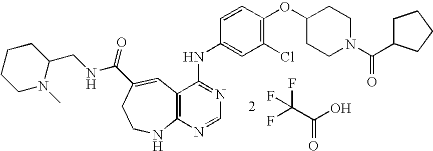

### Production of 4-[(3-chloro-4-{[1-(cyclopentylcarbonyl)piperidin-4-yl]oxy}phenyl)amino]-N-[(1-methylpiperidin-2-yl)methyl]-8,9-dihydro-7H-pyrimido[4,5-b]azepine-6-carboxamide ditrifluoroacetate

Using 0.1 M solution of 4-[(3-chloro-4-{[1-(cyclopentylcarbonyl)piperidin-4-yl]oxy}phenyl)amino]-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of (1-methylpiperidin-2-yl)methaneamine in N,N-dimethylformamide (0.5 mL), a similar reaction as in Example 4 was carried out to give the title compound (38.8 mg).

LC-MS found 622 (M+H)⁺ (exact MS calcd for C₃₃H₄₄ClN₇O₃ 621).

### Example 80

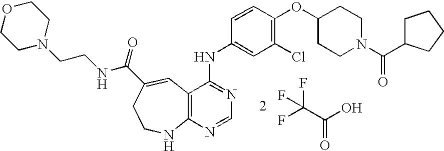

### Production of 4-[(3-chloro-4-{[1-(cyclopentylcarbonyl)piperidin-4-yl]oxy}phenyl)amino]-N-(2-morpholin-4-ylethyl)-8,9-dihydro-7H-pyrimido[4,5-b]azepine-6-carboxamide ditrifluoroacetate

Using 0.1 M solution of 4-[(3-chloro-4-{[1-(cyclopentylcarbonyl)piperidin-4-yl]oxy}phenyl)amino]-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of 4-(2-aminoethyl)morpholine in N,N-dimethylformamide (0.5 mL), a similar reaction as in Example 4 was carried out to give the title compound (37.5 mg).

LC-MS found 624 (M+H)⁺ (exact MS calcd for C₃₂H₄₂ClN₇O₄ 623).

### Example 81

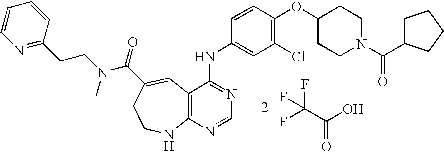

### Production of 4-[(3-chloro-4-{[1-(cyclopentylcarbonyl)piperidin-4-yl]oxy}phenyl)amino]-N-methyl-N-(2-pyridin-2-ylethyl)-8,9-dihydro-7H-pyrimido[4,5-b]azepine-6-carboxamide ditrifluoroacetate

Using 0.1 M solution of 4-[(3-chloro-4-{[1-(cyclopentylcarbonyl)piperidin-4-yl]oxy}phenyl)amino]-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of 2-[2-(methylamino)ethyl]pyridine in N,N-dimethylformamide (0.5 mL), a similar reaction as in Example 4 was carried out to give the title compound (34.8 mg).

LC-MS found 630 (M+H)⁺ (exact MS calcd for C₃₄H₄₀ClN₇O₃ 629).

### Example 82

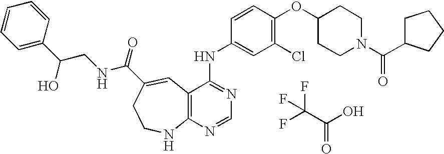

### Production of 4-[(3-chloro-4-{[1-(cyclopentylcarbonyl)piperidin-4-yl]oxy}phenyl)amino]-N-(2-hydroxy-2-phenylethyl)-8,9-dihydro-7H-pyrimido[4,5-b]azepine-6-carboxamide trifluoroacetate

Using 0.1 M solution of 4-[(3-chloro-4-{[1-(cyclopentylcarbonyl)piperidin-4-yl]oxy}phenyl)amino]-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of 2-amino-1-phenylethanol in N,N-dimethylformamide (0.5 mL), a similar reaction as in Example 4 was carried out to give the title compound (24.6 mg).

LC-MS found 631 (M+H)⁺ (exact MS calcd for C₃₄H₃₉ClN₆O₄ 630).

### Example 83

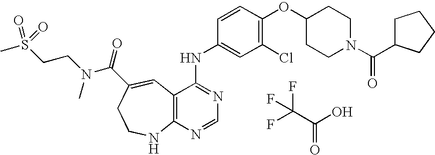

### Production of 4-[(3-chloro-4-{[1-(cyclopentylcarbonyl)piperidin-4-yl]oxy}phenyl)amino]-N-methyl-N-[2-(methylsulfonyl)ethyl]-8,9-dihydro-7H-pyrimido[4,5-b]azepine-6-carboxamide trifluoroacetate

Using 0.1 M solution of 4-[(3-chloro-4-{[1-(cyclopentylcarbonyl)piperidin-4-yl]oxy}phenyl)amino]-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of N-methyl-2-(methylsulfonyl)ethylamine in N,N-dimethylformamide (0.5 mL), a similar reaction as in Example 4 was carried out to give the title compound (29.2 mg).

LC-MS found 631 (M+H)⁺ (exact MS calcd for C₃₀H₃₉ClN₆O₅S 630).

### Example 84

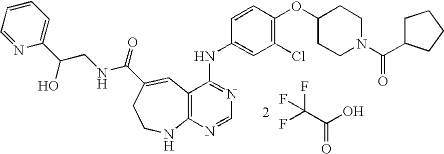

### Production of 4-[(3-chloro-4-{[1-(cyclopentylcarbonyl)piperidin-4-yl]oxy}phenyl)amino]-N-(2-hydroxy-2-pyridin-2-ylethyl)-8,9-dihydro-7H-pyrimido[4,5-b]azepine-6-carboxamide ditrifluoroacetate

Using 0.1 M solution of 4-[(3-chloro-4-{[1-(cyclopentylcarbonyl)piperidin-4-yl]oxy}phenyl)amino]-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of 2-amino-1-pyridin-2-ylethanol in N,N-dimethylformamide (0.5 mL), a similar reaction as in Example 4 was carried out to give the title compound (27.7 mg).

LC-MS found 632 (M+H)⁺ (exact MS calcd for C₃₃H₃₈ClN₇O₄ 631).

### Example 85

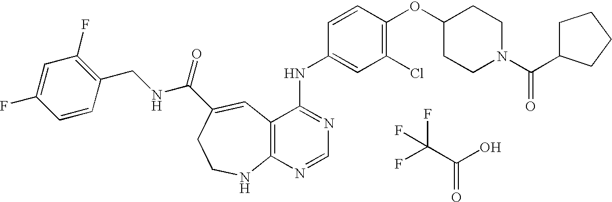

### Production of 4-[(3-chloro-4-{[1-(cyclopentylcarbonyl)piperidin-4-yl]oxy}phenyl)amino]-N-(2,4-difluorobenzyl)-8,9-dihydro-7H-pyrimido[4,5-b]azepine-6-carboxamide trifluoroacetate

Using 0.1 M solution of 4-[(3-chloro-4-{[1-(cyclopentylcarbonyl)piperidin-4-yl]oxy}phenyl)amino]-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of 2,4-difluorobenzylamine in N,N-dimethylformamide (0.5 mL), a similar reaction, as in Example 4 was carried out to give the title compound (28.9 mg).

LC-MS found 637 (M+H)⁺ (exact MS calcd for C₃₃H₃₅ClF₂N₆O₃ 636).

### Example 86

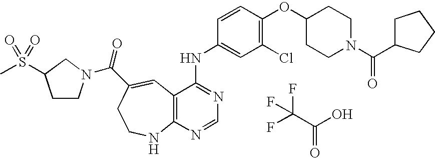

### Production of N-(3-chloro-4-{[1-(cyclopentylcarbonyl)piperidin-4-yl]oxy}phenyl)-6-{[3-(methylsulfonyl)pyrrolidin-1-yl]carbonyl}-8,9-dihydro-7H-pyrimido[4,5-b]azepin-4-amine trifluoroacetate

Using 0.1 M solution of 4-[(3-chloro-4-{[1-(cyclopentylcarbonyl)piperidin-4-yl]oxy}phenyl)amino]-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of 3-(methylsulfonyl)pyrrolidine in N,N-dimethylformamide (0.5 mL), a similar reaction as in Example 4 was carried out to give the title compound (30.1 mg).

LC-MS found 643 (M+H)⁺ (exact MS calcd for C₃₁H₃₉ClN₆O₅S 642).

### Example 87

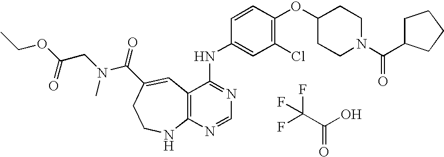

Production of ethyl N-({4-[(3-chloro-4-{[1-(cyclopentylcarbonyl)piperidin-4-yl]oxy}phenyl)amino]-8,9-dihydro-7H-pyrimido[4,5-b]azepin-6-yl}carbonyl)-N-methylglycinate trifluoroacetate

Using 0.1 M solution of 4-[(3-chloro-4-{[1-(cyclopentylcarbonyl)piperidin-4-yl]oxy}phenyl)amino]-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL), 0.15 M solution of ethyl N-methylglycinate hydrochloride in N,N-dimethylformamide (0.5 mL) and triethylamine (0.01 mL), a similar reaction as in Example 7 was carried out to give the title compound (26.7 mg).

LC-MS found 611 (M+H)⁺ (exact MS calcd for C₃₁H₃₉ClN₆O₅ 610).

### Example 88

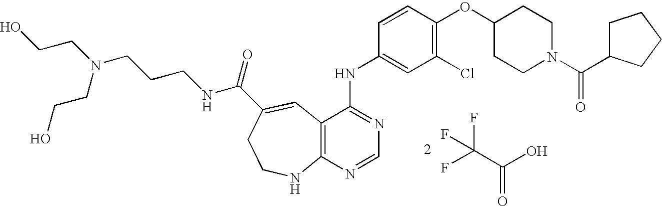

### Production of N-{3-[bis(2-hydroxyethyl)amino]propyl}-4-[(3-chloro-4-{[1-(cyclopentylcarbonyl)piperidin-4-yl]oxy}phenyl)amino]-8,9-dihydro-7H-pyrimido[4,5-b]azepine-6-carboxamide ditrifluoroacetate

Using 0.1 M solution of 4-[(3-chloro-4-{[1-(cyclopentylcarbonyl)piperidin-4-yl]oxy}phenyl)amino]-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of 2,2′-[(3-aminopropyl)imino]diethanol in N,N-dimethylformamide (0.5 mL), a similar reaction as in Example 4 was carried out to give the title compound (37.7 mg).

LC-MS found 656 (M+H)⁺ (exact MS calcd for C₃₃H₄₆ClN₇O₅ 655).

### Example 89

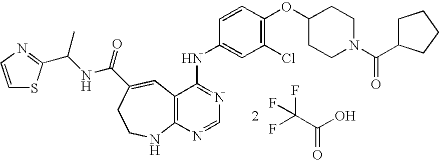

### Production of 4-[(3-chloro-4-{[1-(cyclopentylcarbonyl)piperidin-4-yl]oxy}phenyl)amino]-N-[1-(1,3-thiazol-2-yl)ethyl]-8,9-dihydro-7H-pyrimido[4,5-b]azepine-6-carboxamide ditrifluoroacetate

Using 0.1 M solution of 4-[(3-chloro-4-{[1-(cyclopentylcarbonyl)piperidin-4-yl]oxy}phenyl)amino]-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL), 0.15 M solution of 1-(1,3-thiazol-2-yl)ethylamine hydrochloride in N,N-dimethylformamide (0.5 mL) and triethylamine (0.01 mL), a similar reaction as in Example 7 was carried out to give the title compound (28.1 mg).

LC-MS found 622 (M+H)⁺ (exact MS calcd for C₃₁H₃₆ClN₇O₃S 621).

### Example 90

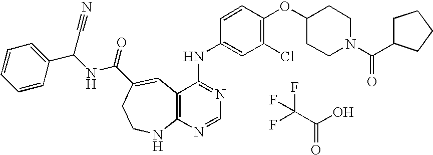

### Production of 4-[(3-chloro-4-{[1-(cyclopentylcarbonyl)piperidin-4-yl]oxy}phenyl)amino]-N-[cyano(phenyl)methyl]-8,9-dihydro-7H-pyrimido[4,5-b]azepine-6-carboxamide trifluoroacetate

Using 0.1 M solution of 4-[(3-chloro-4-{[1-(cyclopentylcarbonyl)piperidin-4-yl]oxy}phenyl)amino]-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL), 0.15 M solution of amino(phenyl)acetonitrile hydrochloride in N,N-dimethylformamide (0.5 mL) and triethylamine (0.01 mL), a similar reaction as in Example 7 was carried out to give the title compound (26.8 mg).

LC-MS found 626 (M+H)⁺ (exact MS calcd for C₃₄H₃₆ClN₇O₃ 625).

### Example 91

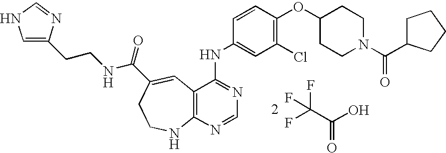

### Production of 4-[(3-chloro-4-{[1-(cyclopentylcarbonyl)piperidin-4-yl]oxy}phenyl)amino]-N-[2-(1H-imidazol-4-yl)ethyl]-8,9-dihydro-7H-pyrimido[4,5-b]azepine-6-carboxamide ditrifluoroacetate

Using 0.1 M solution of 4-[(3-chloro-4-{[1-(cyclopentylcarbonyl)piperidin-4-yl]oxy}phenyl)amino]-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL), 0.15 M solution of 2-(1H-imidazol-4-yl)ethylamine dihydrochloride in N,N-dimethylformamide (0.5 mL) and triethylamine (0.02 mL), a similar reaction as in Example 7 was carried out to give the title compound (22.9 mg).

LC-MS found 605 (M+H)⁺ (exact MS calcd for C₃₁H₃₇ClN₈O₃ 604).

### Example 92

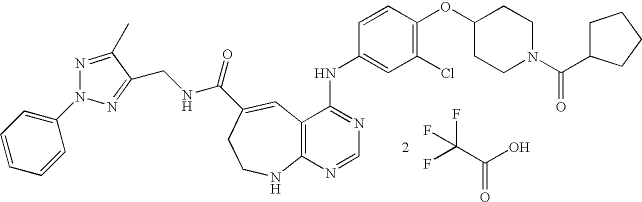

### Production of 4-[(3-chloro-4-{[1-(cyclopentylcarbonyl)piperidin-4-yl]oxy}phenyl)amino]-N-[(5-methyl-2-phenyl-2H-1,2,3-triazol-4-yl)methyl]-8,9-dihydro-7H-pyrimido[4,5-b]azepine-6-carboxamide ditrifluoroacetate

Using 0.1 M solution of 4-[(3-chloro-4-{[1-(cyclopentylcarbonyl)piperidin-4-yl]oxy}phenyl)amino]-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of 1-(5-methyl-2-phenyl-2H-1,2,3-triazol-4-yl)methylamine in N,N-dimethylformamide (0.5 mL), a similar reaction as in Example 4 was carried out to give the title compound (31.1 mg).

LC-MS found 682 (M+H)⁺ (exact MS calcd for C₃₆H₄₀ClN₉O₃ 681).

### Example 93

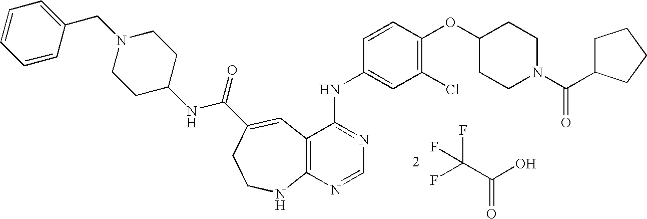

### Production of N-(1-benzylpiperidin-4-yl)-4-[(3-chloro-4-{[1-(cyclopentylcarbonyl)piperidin-4-yl]oxy}phenyl)amino]-8,9-dihydro-7H-pyrimido[4,5-b]azepine-6-carboxamide ditrifluoroacetate

Using 0.1 M solution of 4-[(3-chloro-4-{[1-(cyclopentylcarbonyl)piperidin-4-yl]oxy}phenyl)amino]-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of 4-amino-1-benzylpiperidine in N,N-dimethylformamide (0.5 mL), a similar reaction as in Example 4 was carried out to give the title compound (41.6 mg).

LC-MS found 684 (M+H)⁺ (exact MS calcd for C₃₈H₄₆ClN₇O₃ 683).

### Example 94

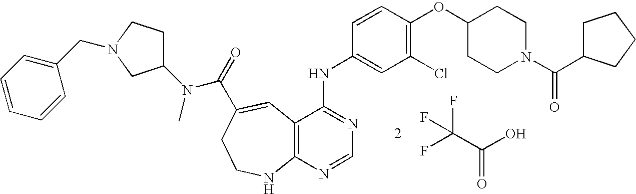

### Production of N-(1-benzylpyrrolidin-3-yl)-4-[(3-chloro-4-{[1-(cyclopentylcarbonyl)piperidin-4-yl]oxy}phenyl)amino]-N-methyl-8,9-dihydro-7H-pyrimido[4,5-b]azepine-6-carboxamide ditrifluoroacetate

Using 0.1 M solution of 4-[(3-chloro-4-{[1-(cyclopentylcarbonyl)piperidin-4-yl]oxy}phenyl)amino]-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of 1-benzyl-3-(methylamino)pyrrolidine in N,N-dimethylformamide (0.5 mL), a similar reaction as in Example 4 was carried out to give the title compound (46.8 mg).

LC-MS found 684 (M+H)⁺ (exact MS calcd for C₃₈H₄₆ClN₇O₃ 683).

### Example 95

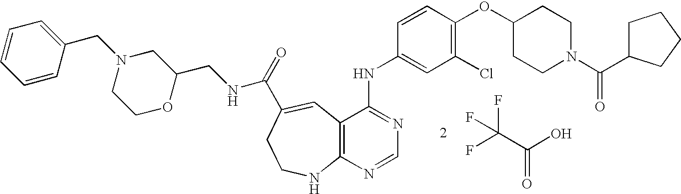

### Production of N-[(4-benzylmorpholin-2-yl)methyl]-4-[(3-chloro-4-{[1-(cyclopentylcarbonyl)piperidin-4-yl]oxy}phenyl)amino]-8,9-dihydro-7H-pyrimido[4,5-b]azepine-6-carboxamide ditrifluoroacetate

Using 0.1 M solution of 4-[(3-chloro-4-{[1-(cyclopentylcarbonyl)piperidin-4-yl]oxy}phenyl)amino]-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL) and 0.15 M solution of 1-(4-benzylmorpholin-2-yl)methylamine in N,N-dimethylformamide (0.5 mL), a similar reaction as in Example 4 was carried out to give the title compound (39.2 mg).

LC-MS found 700 (M+H)⁺ (exact MS calcd for C₃₈H₄₆ClN₇O₄ 699).

### Example 96

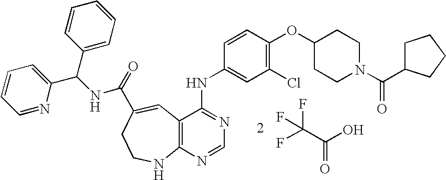

### Production of 4-[(3-chloro-4-{[1-(cyclopentylcarbonyl)piperidin-4-yl]oxy}phenyl)amino]-N-[phenyl(pyridin-2-yl)methyl]-8,9-dihydro-7H-pyrimido[4,5-b]azepine-6-carboxamide ditrifluoroacetate

Using 0.1 M solution of 4-[(3-chloro-4-{[1-(cyclopentylcarbonyl)piperidin-4-yl]oxy}phenyl)amino]-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL), 0.15 M solution of 1-phenyl-1-pyridin-2-ylmethylamine hydrochloride in N,N-dimethylformamide (0.5 mL) and triethylamine (0.01 mL), a similar reaction as in Example 7 was carried out to give the title compound (39.4 mg).

LC-MS found 678 (M+H)⁺ (exact MS calcd for C₃₈H₄₀ClN₇O₃ 677).

### Example 97

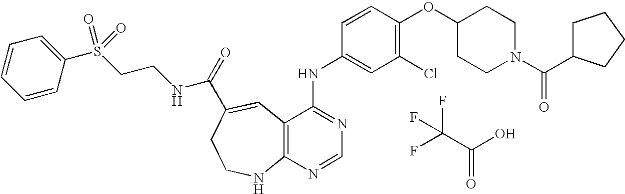

### Production of 4-[(3-chloro-4-{[1-(cyclopentylcarbonyl)piperidin-4-yl]oxy}phenyl)amino]-N-[2-(phenylsulfonyl)ethyl]-8,9-dihydro-7H-pyrimido[4,5-b]azepine-6-carboxamide trifluoroacetate

Using 0.1 M solution of 4-[(3-chloro-4-{[1-(cyclopentylcarbonyl)piperidin-4-yl]oxy}phenyl)amino]-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL), 0.15 M solution of 2-(phenylsulfonyl)ethylamine hydrochloride in N,N-dimethylformamide (0.5 mL) and triethylamine (0.01 mL), a similar reaction as in Example 7 was carried out to give the title compound (20.8 mg).

LC-MS found 679 (M+H)⁺ (exact MS calcd for C₃₄H₃₉ClN₆O₅S 678).

### Example 98

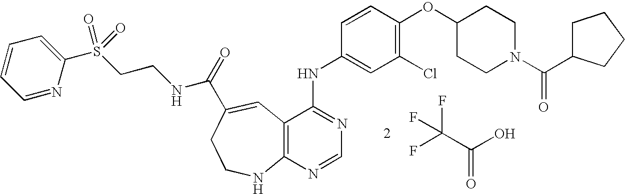

### Production of 4-[(3-chloro-4-{[1-(cyclopentylcarbonyl)piperidin-4-yl]oxy}phenyl)amino]-N-[2-(pyridin-2-ylsulfonyl)ethyl]-8,9-dihydro-7H-pyrimido[4,5-b]azepine-6-carboxamide ditrifluoroacetate

Using 0.1 M solution of 4-[(3-chloro-4-{[1-(cyclopentylcarbonyl)piperidin-4-yl]oxy}phenyl)amino]-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL), 0.15 M solution of 2-[(pyridin-2-yl)sulfonyl]ethylamine hydrochloride in N,N-dimethylformamide (0.5 mL) and triethylamine (0.01 mL), a similar reaction as in Example 7 was carried out to give the title compound (46.5 mg).

LC-MS found 680 (M+H)⁺ (exact MS calcd for C₃₃H₃₈ClN₇O₅S 679).

### Example 99

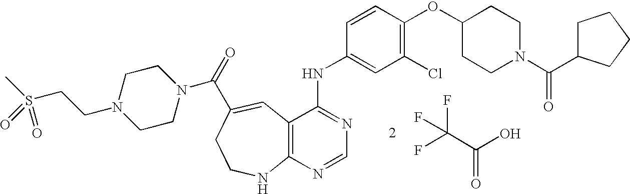

### Production of N-(3-chloro-4-{[1-(cyclopentylcarbonyl)piperidin-4-yl]oxy}phenyl)-6-({4-[2-(methylsulfonyl)ethyl]piperazin-1-yl}carbonyl)-8,9-dihydro-7H-pyrimido[4,5-b]azepin-4-amine ditrifluoroacetate

Using 0.1 M solution of 4-[(3-chloro-4-{[1-(cyclopentylcarbonyl)piperidin-4-yl]oxy}phenyl)amino]-8,9-dihydro-7H-pyrimido[4,5-b]azepine-6-carboxylic acid in N,N-dimethylformamide (0.5 mL), 0.15 M solution of 1-hydroxybenzotriazole/1-ethyl-3-(3-dimethylaminopropyl)carbodiimide hydrochloride=1/1 in N,N-dimethylformamide (0.5 mL), 0.15 M solution of 1-[2-(methylsulfonyl)ethyl]piperazine hydrochloride in N,N-dimethylformamide (0.5 mL) and triethylamine (0.01 mL), a similar reaction as in Example 7 was carried out to give the title compound (32.7 mg).

LC-MS found 686 (M+H)⁺ (exact MS calcd for C₃₃H₄₄ClN₇O₅S 685).

### Example 100

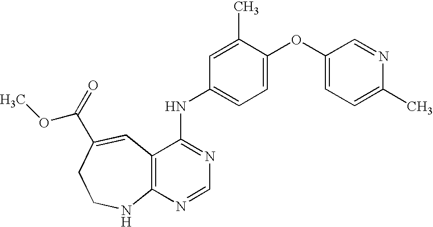

### Production of methyl 4-({3-methyl-4-[(6-methylpyridin-3-yl)oxy]phenyl}amino)-8,9-dihydro-7H-pyrimido[4,5-b]azepine-6-carboxylate

### (i) Production of methyl 4-[[5-formyl-6-({3-methyl-4-[(6-methylpyridin-3-yl)oxy]phenyl}amino)pyrimidin-4-yl](4-methoxybenzyl)amino]butanoate

A mixture of methyl 4-[(6-chloro-5-formylpyrimidin-4-yl)(4-methoxybenzyl)amino]butanoate (2.50 g), 3-methyl-4-[(6-methylpyridin-3-yl)oxy]aniline (1.42 g) and potassium carbonate (0.92 g) in N,N-dimethylformamide (25 mL) was stirred at room temperature for 6 days. Water was added to the reaction mixture, and the mixture was extracted with ethyl acetate. The organic layer was washed with water and saturated brine, dried over anhydrous magnesium sulfate and concentrated under reduced pressure. The residue was separated and purified by column chromatography (eluent, hexane:ethyl acetate=3:1→1:3) to give the title compound (3.08 g) as pale-yellow amorphous.

¹H-NMR (CDCl₃) δ: 1.96-2.10 (2H, m), 2.25 (3H, s), 2.33 (2H, t, J=7.2 Hz), 2.52 (3H, s), 3.56 (2H, t, J=7.4 Hz), 3.65 (3H, s), 3.81 (3H, s), 4.79 (2H, s), 6.85-6.90 (3H, m), 7.05-7.11 (2H, m), 7.16 (2H, d, J=8.4 Hz), 7.45-7.51 (2H, m), 8.24-8.26 (1H, m), 8.29 (1H, s), 9.80 (1H, s), 11.07 (1H, s).

### (ii) Production of methyl 9-(4-methoxybenzyl)-4-({3-methyl-4-[(6-methylpyridin-3-yl)oxy]phenyl}amino)-8,9-dihydro-7H-pyrimido[4,5-b]azepine-6-carboxylate

To a solution of methyl 4-[[5-formyl-6-({3-methyl-4-[(6-methylpyridin-3-yl)oxy]phenyl}amino)pyrimidin-4-yl](4-methoxybenzyl)amino]butanoate (3.08 g) in dimethyl carbonate (30 mL) was added 28% sodium methoxide-methanol solution (3.20 g), and the mixture was stirred at room temperature for 12 hr and at 50° C. for 3 hr. Water was added to the reaction mixture, and the mixture was extracted with ethyl acetate. The organic layer was washed with aqueous sodium bicarbonate solution and saturated brine, dried over anhydrous magnesium sulfate and concentrated under reduced pressure. The residue was separated and purified by column chromatography (eluent, hexane:ethyl acetate=1:1→ethyl acetate) to give the title compound (2.79 g) as yellow amorphous.

¹H-NMR (CDCl₃) δ: 2.25 (3H, s), 2.53 (3H, s), 2.64 (2H, t, J=4.8 Hz), 3.35 (2H, t, J=4.8 Hz), 3.78 (3H, s), 3.81 (3H, s), 4.86 (2H, s), 6.77 (1H, s), 6.86-6.90 (3H, m), 7.06-7.13 (2H, m), 7.18-7.30 (3H, m), 7.37 (1H, d, J=2.4 Hz), 7.70 (1H, s), 8.22 (1H, s), 8.27 (1H, s).

### (iii) Production of methyl 4-({3-methyl-4-[(6-methylpyridin-3-yl)oxy]phenyl}amino)-8,9-dihydro-7H-pyrimido[4,5-b]azepine-6-carboxylate

Using methyl 9-(4-methoxybenzyl)-4-({3-methyl-4-[(6-methylpyridin-3-yl)oxy]phenyl}amino)-8,9-dihydro-7H-pyrimido[4,5-b]azepine-6-carboxylate (450 mg), trifluoroacetic acid (5.0 mL) and 1,2-dichloroethane (5.0 mL), a similar reaction as in Example 1 (iii) was carried out to give the title compound (308 mg) as colorless crystals.

¹H-NMR (CDCl₃) δ: 2.25 (3H, s), 2.52 (3H, s), 2.90 (2H, t, J=4.8 Hz), 3.52 (2H, q, J=4.8 Hz), 3.81 (3H, s), 5.72-5.80 (1H, m), 6.77 (1H, br s), 6.86 (1H, d, J=8.7 Hz), 7.05-7.11 (2H, m), 7.21-7.28 (1H, m), 7.34 (1H, d, J=2.4 Hz), 7.68 (1H, s), 8.08 (1H, s), 8.24-8.25 (1H, m).

### Example 101

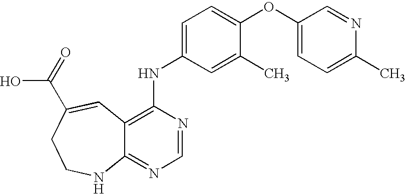

### Production of 4-({3-methyl-4-[(6-methylpyridin-3-yl)oxy]phenyl}amino)-8,9-dihydro-7H-pyrimido[4,5-b]azepine-6-carboxylic acid

To a solution of methyl 4-({3-methyl-4-[(6-methylpyridin-3-yl)oxy]phenyl}amino)-8,9-dihydro-7H-pyrimido[4,5-b]azepine-6-carboxylate (200 mg) in tetrahydrofuran (10 mL) and ethanol (10 mL) was added 1N aqueous sodium hydroxide solution (1.5 mL) at room temperature. The mixture was stirred at room temperature for 4 hr, 1N hydrochloric acid (1.5 mL) was added thereto, and the mixture was concentrated under reduced pressure. The precipitated crystals were collected by filtration. The crystals were washed with ethanol and water to give the title compound (175 mg) as pale-yellow crystals.

¹H-NMR (DMSO-d₆) δ: 2.11 (3H, s), 2.41 (3H, s), 2.63-2.72 (2H, m), 3.24-3.38 (2H, m), 6.85 (1H, d, J=8.7 Hz), 7.12 (1H, dd, J=8.7, 2.7 Hz), 7.19 (1H, d, J=8.7 Hz), 7.31 (1H, dd, J=8.7, 2.7 Hz), 7.40 (1H, d, J= 2.7 Hz), 7.68 (1H, s), 7.71-7.78 (1H, m), 7.89 (1H, s), 8.12 (1H, d, J=2.7 Hz), 9.13 (1H, s), 12.16 (1H, br s).

### Example 102

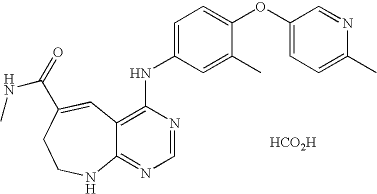

### Production of N-methyl-4-({3-methyl-4-[(6-methylpyridin-3-yl)oxy]phenyl}amino)-8,9-dihydro-7H-pyrimido[4,5-b]azepine-6-carboxamide formate

To 0.12 M solution of 4-({3-methyl-4-[(6-methylpyridin-3-yl)oxy]phenyl}amino)-8,9-dihydro-7H-pyrimido[4,5-b]azepine-6-carboxylic acid in N,N-dimethylformamide (500 μL) were added 0.12 M solution of methylamine hydrochloride in N,N-dimethylformamide (500 μL) and 0.18 M solution of 1-hydroxybenzotriazole/1-ethyl-3-(3-dimethylaminopropyl)carbodiimide hydrochloride=1/1 in N,N-dimethylformamide (500 μL), and the mixture was stirred at room temperature overnight. The reaction mixture was charged to MP-TosH column, and washed with ethyl acetate (2×2.0 mL) and methanol (2×2.0 mL). The solution containing the objective substance was collected by using 1 M ammonia-methanol solution (2.0 mL). The solution was concentrated under reduced pressure and the residue was separated by liquid chromatography (column: C18, mobile phase: 0.1% formic acid containing acetonitrile/0.1% formic acid containing water, 5%->95%). The objective fraction was concentrated under reduced pressure to give the title compound (6.6 mg).

LC-MS found 417 (M+H)⁺ (exact MS calcd for C₂₃H₂₄N₆O₂ 416)

### Example 103

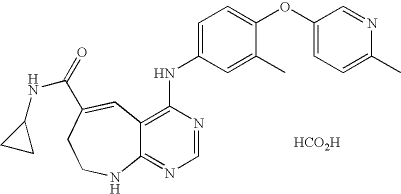

### Production of N-cyclopropyl-4-({3-methyl-4-[(6-methylpyridin-3-yl)oxy]phenyl}amino)-8,9-dihydro-7H-pyrimido[4,5-b]azepine-6-carboxamide formate

Using 0.12 M solution of 4-({3-methyl-4-[(6-methylpyridin-3-yl)oxy]phenyl}amino)-8,9-dihydro-7H-pyrimido[4,5-b]azepine-6-carboxylic acid in N,N-dimethylformamide (500 μL), 0.12 M solution of cyclopropylamine in N,N-dimethylformamide (500 μL) and 0.18 M solution of 1-hydroxybenzotriazole/1-ethyl-3-(3-dimethylaminopropyl)carbodiimide hydrochloride=1/1 in N,N-dimethylformamide (500 μL), a similar reaction as in Example 102 was carried out to give the title compound (6.1 mg).

LC-MS found 443 (M+H)⁺ (exact MS calcd for C₂₅H₂₆N₆O₂ 442)

### Example 104

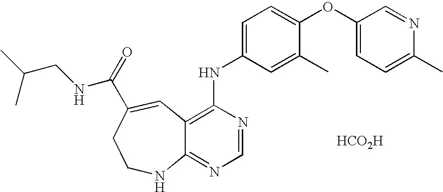

### Production of N-isobutyl-4-({3-methyl-4-[(6-methylpyridin-3-yl)oxy]phenyl}amino)-8,9-dihydro-7H-pyrimido[4,5-b]azepine-6-carboxamide formate

Using 0.12 M solution of 4-({3-methyl-4-[(6-methylpyridin-3-yl)oxy]phenyl}amino)-8,9-dihydro-7H-pyrimido[4,5-b]azepine-6-carboxylic acid in N,N-dimethylformamide (500 μL), 0.12 M solution of isobutylamine in N,N-dimethylformamide (500 μL) and 0.18 M solution of 1-hydroxybenzotriazole/1-ethyl-3-(3-dimethylaminopropyl)carbodiimide hydrochloride=1/1 in N,N-dimethylformamide (500 μL), a similar reaction as in Example 102 was carried out to give the title compound (11.9 mg).

LC-MS found 459 (M+H)⁺ (exact MS calcd for C₂₆H₃₀N₆O₂ 458)

### Example 105

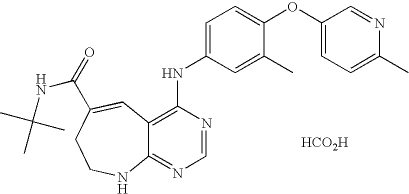

### Production of N-(tert-butyl)-4-({3-methyl-4-[(6-methylpyridin-3-yl)oxy]phenyl}amino)-8,9-dihydro-7H-pyrimido[4,5-b]azepine-6-carboxamide formate

Using 0.12 M solution of 4-({3-methyl-4-[(6-methylpyridin-3-yl)oxy]phenyl}amino)-8,9-dihydro-7H-pyrimido[4,5-b]azepine-6-carboxylic acid in N,N-dimethylformamide (500 μL), 0.12 M solution of tert-butylamine in N,N-dimethylformamide (500 μL) and 0.18 M solution of 1-hydroxybenzotriazole/1-ethyl-3-(3-dimethylaminopropyl)carbodiimide hydrochloride=1/1 in N,N-dimethylformamide (500 μL), a similar reaction as in Example 102 was carried out to give the title compound (3.5 mg). LC-MS found 459 (M+H)⁺ (exact MS calcd for C₂₆H₃₀N₆O₂ 458)

### Example 106

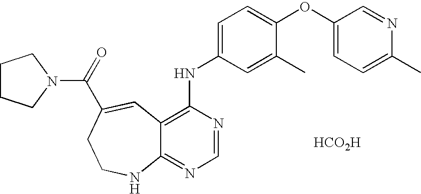

### Production of N-{3-methyl-4-[(6-methylpyridin-3-yl)oxy]phenyl}-6-(pyrrolidin-1-ylcarbonyl)-8,9-dihydro-7H-pyrimido[4,5-b]azepin-4-amine formate

Using 0.12 M solution of 4-({3-methyl-4-[(6-methylpyridin-3-yl)oxy]phenyl}amino)-8,9-dihydro-7H-pyrimido[4,5-b]azepine-6-carboxylic acid in N,N-dimethylformamide (500 μL), 0.12 M solution of pyrrolidine in N,N-dimethylformamide (500 μL) and 0.18 M solution of 1-hydroxybenzotriazole/1-ethyl-3-(3-dimethylaminopropyl)carbodiimide hydrochloride=1/1 in N,N-dimethylformamide (500 μL), a similar reaction as in Example 102 was carried out to give the title compound (11.3 mg).

LC-MS found 457 (M+H)⁺ (exact MS calcd for C₂₆H₂₈N₆O₂ 456)

### Example 107

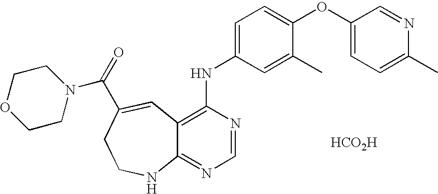

### Production of N-{3-methyl-4-[(6-methylpyridin-3-yl)oxy]phenyl}-6-(morpholin-4-ylcarbonyl)-8,9-dihydro-7H-pyrimido[4,5-b]azepin-4-amine formate

Using 0.12 M solution of 4-({3-methyl-4-[(6-methylpyridin-3-yl)oxy]phenyl}amino)-8,9-dihydro-7H-pyrimido[4,5-b]azepine-6-carboxylic acid in N,N-dimethylformamide (500 μL), 0.12 M solution of morpholine in N,N-dimethylformamide (500 μL) and 0.18 M solution of 1-hydroxybenzotriazole/1-ethyl-3-(3-dimethylaminopropyl)carbodiimide hydrochloride=1/1 in N,N-dimethylformamide (500 μL), a similar reaction as in Example 102 was carried out to give the title compound (7.6 mg).

LC-MS found 473 (M+H)⁺ (exact MS calcd for C₂₆H₂₈N₆O₃ 472)

### Example 108

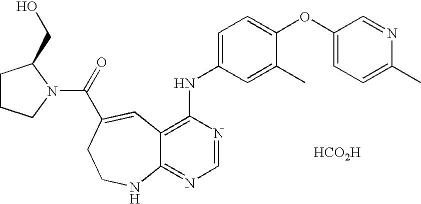

### Production of ((2S)-1-{[4-({3-methyl-4-[(6-methylpyridin-3-yl)oxy]phenyl}amino)-8,9-dihydro-7H-pyrimido[4,5-b]azepin-6-yl]carbonyl}pyrrolidin-2-yl)methanol formate

Using 0.12 M solution of 4-({3-methyl-4-[(6-methylpyridin-3-yl)oxy]phenyl}amino)-8,9-dihydro-7H-pyrimido[4,5-b]azepine-6-carboxylic acid in N,N-dimethylformamide (500 μL), 0.12 M solution of (2S)-pyrrolidine-2-methanol in N,N-dimethylformamide (500 μL) and 0.18 M solution of 1-hydroxybenzotriazole/1-ethyl-3-(3-dimethylaminopropyl)carbodiimide hydrochloride=1/1 in N,N-dimethylformamide (500 μL), a similar reaction as in Example 102 was carried out to give the title compound (9.1 mg).

LC-MS found 487 (M+H)⁺ (exact MS calcd for C₂₇H₃₀N₆O₃ 486)

### Example 109

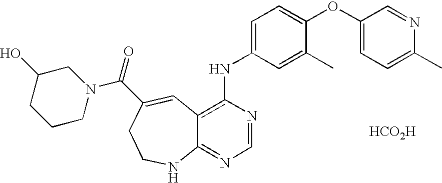

### Production of 1-{[4-({3-methyl-4-[(6-methylpyridin-3-yl)oxy]phenyl}amino)-8,9-dihydro-7H-pyrimido[4,5-b]azepin-6-yl]carbonyl}piperidin-3-ol formate

Using 0.12 M solution of 4-({3-methyl-4-[(6-methylpyridin-3-yl)oxy]phenyl}amino)-8,9-dihydro-7H-pyrimido[4,5-b]azepine-6-carboxylic acid in N,N-dimethylformamide (500 μL), 0.12 M solution of 3-piperidinol in N,N-dimethylformamide (500 μL) and 0.18 M solution of 1-hydroxybenzotriazole/1-ethyl-3-(3-dimethylaminopropyl)carbodiimide hydrochloride=1/1 in N,N-dimethylformamide (500 μL), a similar reaction as in Example 102 was carried out to give the title compound (1.1 mg).

LC-MS found 487 (M+H)⁺ (exact MS calcd for C₂₇H₃₀N₆O₃ 486)

### Example 110

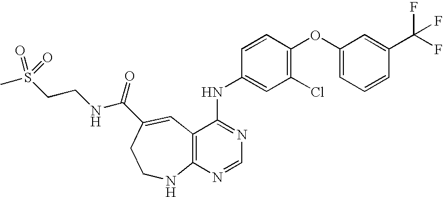

### Production of 4-({3-chloro-4-[3-(trifluoromethyl)phenoxy]phenyl}amino)-N-[2-(methylsulfonyl)ethyl]-8,9-dihydro-7H-pyrimido[4,5-b]azepine-6-carboxamide

To a solution of 4-({3-chloro-4-[3-(trifluoromethyl)phenoxy]phenyl}amino)-8,9-dihydro-7H-pyrimido[4,5-b]azepine-6-carboxylic acid (90.3 mg) in tetrahydrofuran (0.5 mL) and N,N-dimethylformamide (0.5 mL) were successively added 2-(methylsulfonyl)ethylamine (34.6 mg), 1-hydroxybenzotriazole (39.1 mg), triethylamine (0.08 mL) and 1-ethyl-3-(3-dimethylaminopropyl)carbodiimide hydrochloride (107 mg), and the mixture was stirred at room temperature for 2 hr. Water was added to the reaction mixture, and the mixture was extracted with ethyl acetate. The organic layer was washed with saturated brine, dried over anhydrous magnesium sulfate and concentrated under reduced pressure. The residue was separated and purified by silica gel column chromatography (eluent, ethyl acetate:methanol=100:0→90:10) to give the title compound (44.1 mg) as a white powder.

¹H-NMR (DMSO-d₆) δ: 2.64-2.75 (2H, m), 3.03 (3H, s), 3.27-3.41 (4H, m), 3.53-3.65 (2H, m), 7.14-7.24 (3H, m), 7.27 (1H, d, J=9.1 Hz), 7.46 (1H, d, J=8.0 Hz), 7.55-7.66 (2H, m), 7.73 (1H, t, J=4.7 Hz), 7.92 (1H, d, J=2.8 Hz), 7.99 (1H, s), 8.24 (1H, t, J=5.5 Hz), 9.13 (1H, s).

### Example 111

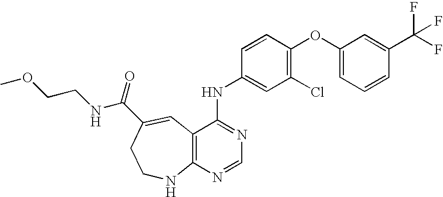

### Production of 4-({3-chloro-4-[3-(trifluoromethyl)phenoxy]phenyl}amino)-N-(2-methoxyethyl)-8,9-dihydro-7H-pyrimido[4,5-b]azepine-6-carboxamide

Using 4-({3-chloro-4-[3-(trifluoromethyl)phenoxy]phenyl}amino)-8,9-dihydro-7H-pyrimido[4,5-b]azepine-6-carboxylic acid (89.4 mg), 2-methoxyethylamine (0.025 mL), 1-hydroxybenzotriazole (39.4 mg), triethylamine (0.08 mL), 1-ethyl-3-(3-dimethylaminopropyl)carbodiimide hydrochloride (115 mg), tetrahydrofuran (0.5 mL) and N,N-dimethylformamide (0.5 mL), a similar reaction as in Example 110 was carried out to give the title compound (59.4 mg) as a white powder.

¹H-NMR (DMSO-d₆) δ: 2.63-2.74 (2H, m), 3.26 (3H, s), 3.29-3.45 (6H, m), 7.13-7.23 (3H, m), 7.26 (1H, d, J=9.1 Hz), 7.42-7.50 (1H, m), 7.55-7.65 (2H, m), 7.68 (1H, t, J=4.5 Hz), 7.89 (1H, d, J=2.8 Hz), 7.98 (1H, s), 8.04 (1H, t, J=5.6 Hz), 9.13 (1H, s).

### Example 112

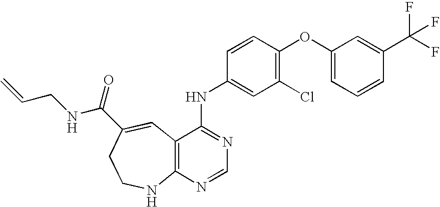

### Production of N-allyl-4-({3-chloro-4-[3-(trifluoromethyl)phenoxy]phenyl}amino)-8,9-dihydro-7H-pyrimido[4,5-b]azepine-6-carboxamide

Using 4-({3-chloro-4-[3-(trifluoromethyl)phenoxy]phenyl}amino)-8,9-dihydro-7H-pyrimido[4,5-b]azepine-6-carboxylic acid (120 mg), allylamine (0.03 mL), 1-hydroxybenzotriazole (52.9 mg), triethylamine (0.1 mL), 1-ethyl-3-(3-dimethylaminopropyl)carbodiimide hydrochloride (148 mg), tetrahydrofuran (0.75 mL) and N,N-dimethylformamide (0.75 mL), a similar reaction as in Example 110 was carried out to give the title compound (70.0 mg) as a white powder.

¹H-NMR (DMSO-d₆) δ: 2.66-2.76 (2H, m), 3.30-3.41 (2H, m), 3.77-3.87 (2H, m), 5.03-5.23 (2H, m), 5.77-5.95 (1H, m), 7.14-7.23 (3H, m), 7.26 (1H, d, J=8.9 Hz), 7.46 (1H, d, J=7.7 Hz), 7.54-7.65 (2H, m), 7.70 (1H, t, J=4.6 Hz), 7.89 (1H, d, J=2.6 Hz), 7.98 (1H, s), 8.17 (1H, t, J=5.7 Hz), 9.13 (1H, s).

### Example 113

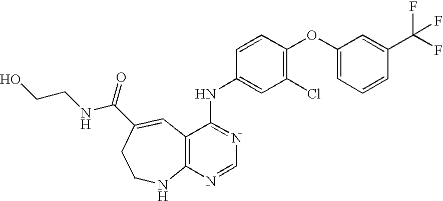

### Production of 4-({3-chloro-4-[3-(trifluoromethyl)phenoxy]phenyl}amino)-N-(2-hydroxyethyl)-8,9-dihydro-7H-pyrimido[4,5-b]azepine-6-carboxamide

Using 4-({3-chloro-4-[3-(trifluoromethyl)phenoxy]phenyl}amino)-8,9-dihydro-7H-pyrimido[4,5-b]azepine-6-carboxylic acid (99.9 mg), 2-aminoethanol (0.02 mL), 1-hydroxybenzotriazole (44.3 mg), triethylamine (0.1 mL), 1-ethyl-3-(3-dimethylaminopropyl)carbodiimide hydrochloride (87.3 mg), tetrahydrofuran (0.5 mL) and N,N-dimethylformamide (0.5 mL), a similar reaction as in Example 110 was carried out to give the title compound (68.8 mg) as a white powder.

¹H-NMR (DMSO-d₆) δ: 2.64-2.72 (2H, m), 3.19-3.29 (2H, m), 3.29-3.39 (2H, m), 3.42-3.52 (2H, m), 4.70 (1H, t, J=5.6 Hz), 7.14-7.23 (3H, m), 7.26 (1H, d, J=8.8 Hz), 7.46 (1H, d, J=8.0 Hz), 7.55-7.71° (3H, m), 7.90 (1H, d, J=2.5 Hz), 7.95-8.03 (2H, m), 9.11 (1H, s).

### Example 114

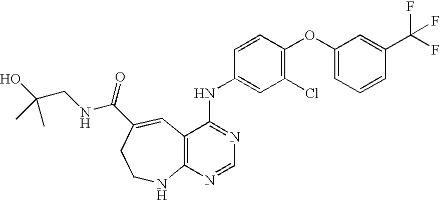

### Production of 4-({3-chloro-4-[3-(trifluoromethyl)phenoxy]phenyl}amino)-N-(2-hydroxy-2-methylpropyl)-8,9-dihydro-7H-pyrimido[4,5-b]azepine-6-carboxamide

Using 4-({3-chloro-4-[3-(trifluoromethyl)phenoxy]phenyl}amino)-8,9-dihydro-7H-pyrimido[4,5-b]azepine-6-carboxylic acid (151 mg), 1-amino-2-methyl-2-propanol (58.0 mg), 1-hydroxybenzotriazole (65.8 mg), triethylamine (0.1 mL), 1-ethyl-3-(3-dimethylaminopropyl)carbodiimide hydrochloride (93.7 mg), tetrahydrofuran (0.7 mL) and N,N-dimethylformamide (0.7 mL), a similar reaction as in Example 110 was carried out to give the title compound (120 mg) as a white powder.

¹H-NMR (DMSO-d₆) δ: 1.26 (6H, s), 2.49 (1H, br s), 2.78-2.86 (2H, m), 3.39 (2H, d, J=6 Hz), 3.51-3.59 (2H, m), 5.79 (1H, t, J=4.7 Hz), 6.33 (1H, t, J=6 Hz), 7.03 (1H, d, J=8.9 Hz), 7.09 (1H, dd, J=8.0 Hz, 2.0 Hz), 7.21 (1H, s), 7.32 (1H, d, J=7.9 Hz), 7.37-7.43 (3H, m), 7.44-8.49 (1H, m), 7.75 (1H, d, J=2.0 Hz), 8.12 (1H, s).

### Example 115

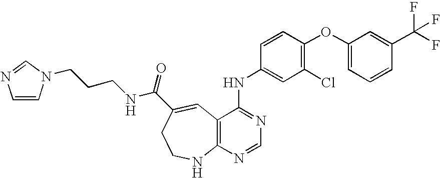

### Production of 4-({3-chloro-4-[3-(trifluoromethyl)phenoxy]phenyl}amino)-N-[3-(1H-imidazol-1-yl)propyl]-8,9-dihydro-7H-pyrimido[4,5-b]azepine-6-carboxamide

Using 4-({3-chloro-4-[3-(trifluoromethyl)phenoxy]phenyl}amino)-8,9-dihydro-7H-pyrimido[4,5-b]azepine-6-carboxylic acid (103 mg), 1-(3-aminopropyl)imidazole (0.035 mL), 1-hydroxybenzotriazole (44.7 mg), triethylamine (0.1 mL), 1-ethyl-3-(3-dimethylaminopropyl)carbodiimide hydrochloride (62.7 mg), tetrahydrofuran (0.5 mL) and N,N-dimethylformamide (0.5 mL), a similar reaction as in Example 110 was carried out to give the title compound (46.8 mg) as a pale-yellow powder.

¹H-NMR (DMSO-d₆) δ: 1.85-1.97 (2H, m), 2.66-2.74 (2H, m), 3.14 (2H, q, J=6.6 Hz), 3.29-3.39 (2H, m), 4.00 (2H, t, J=6.9 Hz), 6.86-6.89 (1H, m), 7.15-7.22 (4H, m), 7.25 (1H, d, J=8.9 Hz), 7.46 (1H, d, J=7.7 Hz), 7.56-7.66 (3H, m), 7.69 (1H, t, J=4.6 Hz), 7.92 (1H, d, J=2.6 Hz), 7.99 (1H, s), 8.05 (1H, t, J=5.6 Hz), 9.19 (1H, s).

### Example 116

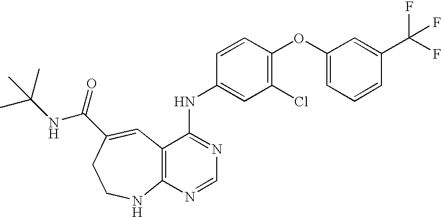

### Production of N-(tert-butyl)-4-({3-chloro-4-[3-(trifluoromethyl)phenoxy]phenyl}amino)-8,9-dihydro-7H-pyrimido[4,5-b]azepine-6-carboxamide

Using 4-({3-chloro-4-[3-(trifluoromethyl)phenoxy]phenyl}amino)-8,9-dihydro-7H-pyrimido[4,5-b]azepine-6-carboxylic acid (101 mg), tert-butylamine (0.03 mL), 1-hydroxybenzotriazole (42.9 mg), triethylamine (0.1 mL), 1-ethyl-3-(3-dimethylaminopropyl)carbodiimide hydrochloride (63.7 mg), tetrahydrofuran (0.5 mL) and N,N-dimethylformamide (0.5 mL), a similar reaction as in Example 110 was carried out to give the title compound (62.6 mg) as a pale-yellow powder.

¹H-NMR (DMSO-d₆) δ: 1.33 (9H, s), 2.61-2.69 (2H, m), 3.27-3.40 (2H, m), 7.01 (1H, s), 7.14-7.24 (2H, m), 7.26 (1H, d, J=8.9 Hz), 7.37 (1H, s), 7.46 (1H, d, J=1.1 Hz), 7.54-7.67 (3H, m), 7.95 (1H, d, J=2.6 Hz), 7.99 (1H, s), 9.16 (1H, s).

### Example 117

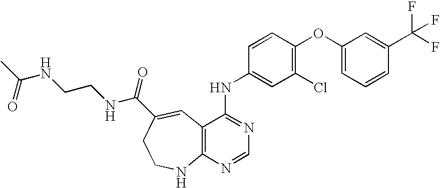

### Production of N-[2-(acetylamino)ethyl]-4-({3-chloro-4-[3-(trifluoromethyl)phenoxy]phenyl}amino)-8,9-dihydro-7H-pyrimido[4,5-b]azepine-6-carboxamide

Using 4-({3-chloro-4-[3-(trifluoromethyl)phenoxy]phenyl}amino)-8,9-dihydro-7H-pyrimido[4,5-b]azepine-6-carboxylic acid (101 mg), N-(2-aminoethyl)acetamide (0.03 mL), 1-hydroxybenzotriazole (44.0 mg), triethylamine (0.1 mL), 1-ethyl-3-(3-dimethylaminopropyl)carbodiimide hydrochloride (59.6 mg), tetrahydrofuran (0.5 mL) and N,N-dimethylformamide (0.5 mL), a similar reaction as in Example 110 was carried out to give the title compound (69.5 mg) as a pale-yellow powder.

¹H-NMR (DMSO-d₆) δ: 1.79 (3H, s), 2.65-2.75 (2H, m), 3.11-3.26 (4H, m), 3.29-3.39 (2H, m), 7.14-7.23 (3H, m), 7.26 (1H, d, J=9.4 Hz), 7.46 (1H, d, J=6.9 Hz), 7.55-7.74 (3H, m), 7.92 (1H, d, J=1.9 Hz), 7.95-8.02 (2H, m), 8.06 (1H, t, J=4.8 Hz), 9.11 (1H, s).

### Example 118

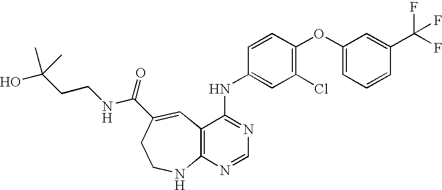

### Production of 4-({3-chloro-4-[3-(trifluoromethyl)phenoxy]phenyl}amino)-N-(3-hydroxy-3-methylbutyl)-8,9-dihydro-7H-pyrimido[4,5-b]azepine-6-carboxamide

Using 4-({3-chloro-4-[3-(trifluoromethyl)phenoxy]phenyl}amino)-8,9-dihydro-7H-pyrimido[4,5-b]azepine-6-carboxylic acid (151 mg), 4-amino-2-methyl-2-butanol hydrochloride (78.2 mg), 1-hydroxybenzotriazole (64.8 mg), triethylamine (0.2 mL), 1-ethyl-3-(3-dimethylaminopropyl)carbodiimide hydrochloride (95.3 mg), tetrahydrofuran (0.7 mL) and N,N-dimethylformamide (0.7 mL), a similar reaction as in Example 110 was carried out to give the title compound (74.0 mg) as a white powder.

¹H-NMR (DMSO-d₆) δ: 1.12 (6H, s), 1.53-1.68 (2H, m), 2.63-2.74 (2H, m), 3.19-3.30 (2H, m), 3.30-3.42 (2H, m), 4.31 (1H, s), 7.11-7.23 (3H, m), 7.26 (1H, d, J=8.9 Hz), 7.46 (1H, d, J=8.1 Hz), 7.54-7.72 (3H, m), 7.85-7.94 (2H, m), 7.98 (1H, s), 9.10 (1H, s).

### Example 119

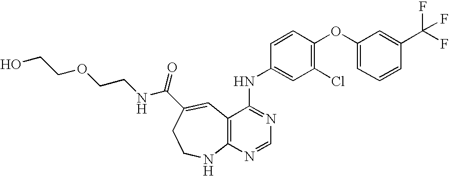

### Production of 4-({3-chloro-4-[3-(trifluoromethyl)phenoxy]phenyl}amino)-N-[2-(2-hydroxyethoxy)ethyl]-8,9-dihydro-7H-pyrimido[4,5-b]azepine-6-carboxamide

Using 4-({3-chloro-4-[3-(trifluoromethyl)phenoxy]phenyl}amino)-8,9-dihydro-7H-pyrimido[4,5-b]azepine-6-carboxylic acid (120 mg), 2-(2-aminoethoxy)ethanol (0.038 mL), 1-hydroxybenzotriazole (51.5 mg), triethylamine (0.1 mL), 1-ethyl-3-(3-dimethylaminopropyl)carbodiimide hydrochloride (71.7 mg), tetrahydrofuran (0.6 mL) and N,N-dimethylformamide (0.6 mL), a similar reaction as in Example 110 was carried out to give the title compound (91.0 mg) as a white powder.

¹H-NMR (DMSO-d₆) δ: 2.65-2.73 (2H, m), 3.28-3.40 (4H, m), 3.40-3.54 (6H, m), 4.58 (1H, t, J=5.4 Hz), 7.14-7.23 (3H, m), 7.26 (1H, d, J=8.9 Hz), 7.46 (1H, d, J=7.7 Hz), 7.55-7.65 (2H, m), 7.68 (1H, t, J=4.7 Hz), 7.90 (1H, d, J=2.5 Hz), 7.98 (1H, s), 8.03 (1H, t, J=5.5 Hz), 9.13 (1H, s).

### Example 120

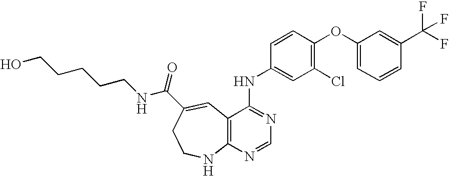

### Production of 4-({3-chloro-4-[3-(trifluoromethyl)phenoxy]phenyl}amino)-N-(5-hydroxypentyl)-8,9-dihydro-7H-pyrimido[4,5-b]azepine-6-carboxamide

Using 4-({3-chloro-4-[3-(trifluoromethyl)phenoxy]phenyl}amino)-8,9-dihydro-7H-pyrimido[4,5-b]azepine-6-carboxylic acid (111 mg), 5-aminopentan-1-ol (45.3 mg), 1-hydroxybenzotriazole (47.7 mg), triethylamine (0.05 mL), 1-ethyl-3-(3-dimethylaminopropyl)carbodiimide hydrochloride (69.5 mg), tetrahydrofuran (0.6 mL) and N,N-dimethylformamide (0.6 mL), a similar reaction as in Example 110 was carried out to give the title compound (81.3 mg) as a pale-yellow powder.

¹H-NMR (DMSO-d₆) δ: 1.24-1.55 (6H, m), 2.64-2.74 (2H, m), 3.09-3.22 (2H, m), 3.29-3.43 (4H, m), 4.35 (1H, t, J=5.1 Hz), 7.12-7.23 (3H, m), 7.26 (1H, d, J=8.8 Hz), 7.46 (1H, d, J=8.0 Hz), 7.56-7.64 (2H, m), 7.67 (1H, t, J=4.7 Hz), 7.91 (1H, d, J=2.7 Hz), 7.93-8.01 (2H, m), 9.14 (1H, s).

### Example 121

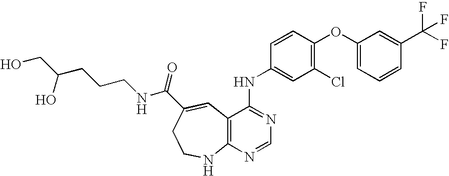

### Production of 4-({3-chloro-4-[3-(trifluoromethyl)phenoxy]phenyl}amino)-N-(4,5-dihydroxypentyl)-8,9-dihydro-7H-pyrimido[4,5-b]azepine-6-carboxamide

### (i) Production of 3-(2,2-dimethyl-1,3-dioxolan-4-yl)propan-1-ol

To the solution of pentane-1,2,5-triol (5.00 g) in acetone (150 mL) were successively added p-toluenesulfonic acid (794 mg) and 2,2-dimethoxypropane (10.5 mL), and the mixture was stirred at room temperature for 1.5 hr. The reaction mixture was concentrated under reduced pressure, and the residue was separated and purified by silica gel column chromatography (eluent, hexane:ethyl acetate=80:20→50:50) to give the title compound (3.79 g) as a colorless oil.

¹H-NMR (CDCl₃) δ: 1.37 (3H, s), 1.42 (3H, s), 1.57-1.77 (4H, m), 2.05 (1H, br s), 3.53 (1H, t, J=7.3 Hz), 3.60-3.77 (2H, m), 4.00-4.21 (2H, m).

### (ii) Production of 3-(2,2-dimethyl-1,3-dioxolan-4-yl)propyl methanesulfonate

3-(2,2-Dimethyl-1,3-dioxolan-4-yl)propan-1-ol (1.00 g) was dissolved in ethyl acetate (20 mL). Triethylamine (2 mL) and methanesulfonyl chloride (0.55 mL) were successively added to the solution, and the mixture was stirred at 0° C. for 1 hr. Water was added to the reaction mixture, and the mixture was extracted with ethyl acetate. The organic layer was washed with saturated brine, dried over anhydrous magnesium sulfate and concentrated under reduced pressure. The residue was separated and purified by silica gel column chromatography (eluent, hexane:ethyl acetate=80:20→34:66) to give the title compound (1.47 g) as a colorless oil.

¹H-NMR (CDCl₃) δ: 1.35 (3H, s), 1.41 (3H, s), 1.59-2.02 (4H, m), 3.01 (3H, s), 3.54 (1H, t, J=6.8 Hz), 3.98-4.20 (2H, m), 4.28 (2H, dt, J=6.2 Hz, 1.8 Hz).

### (iii) Production of N-[3-(2,2-dimethyl-1,3-dioxolan-4-yl)propyl]-N-formylformamide

To the solution of 3-(2,2-dimethyl-1,3-dioxolan-4-yl)propyl methanesulfonate (1.46 g) in N,N-dimethylformamide (20 mL) was added sodium diformylimide (882 mg), the mixture was stirred at 70° C. for 2.5 hr. The reaction mixture was concentrated under reduced pressure. Water was added to the residue, and the mixture was extracted with ethyl acetate. The organic layer was washed with saturated brine, dried over anhydrous magnesium sulfate and concentrated under reduced pressure. The residue was separated and purified by silica gel column chromatography (eluent, hexane:ethyl acetate=80:20→34:66) to give the title compound (772 mg) as a colorless oil.

¹H-NMR (CDCl₃) δ: 1.34 (3H, s), 1.39 (3H, s), 1.45-1.85 (4H, m), 3.50 (1H, t, J=7.3 Hz), 3.58-3.81 (2H, m), 3.93-4.20 (2H, m), 8.84 (2H, s).

### (iv) Production of 5-aminopentane-1,2-diol hydrochloride

N-[3-(2,2-dimethyl-1,3-dioxolan-4-yl)propyl]-N-formylformamide (772 mg) was dissolved in ethanol (20 mL). 6N Hydrochloric acid (6 mL) was added to the solution, and the mixture was stirred at 60° C. for 22 hr. The reaction mixture was concentrated under reduced pressure to give the title compound (552 mg) as an orange oil.

¹H-NMR (DMSO-d₆) δ: 1.15-1.35 (1H, m), 1.40-1.80 (3H, m), 2.68-2.85 (2H, m), 3.15-3.35 (2H, m), 3.35-3.51 (1H, m), 3.71-4.34 (2H, m), 7.92 (3H, br s).

### (v) Production of 4-({3-chloro-4-[3-(trifluoromethyl)phenoxy]phenyl}amino)-N-(4,5-dihydroxypentyl)-8,9-dihydro-7H-pyrimido[4,5-b]azepine-6-carboxamide

Using 4-({3-chloro-4-[3-(trifluoromethyl)phenoxy]phenyl}amino)-8,9-dihydro-7H-pyrimido[4,5-b]azepine-6-carboxylic acid (151 mg), 5-aminopentane-1,2-diol hydrochloride (97.3 mg), 1-hydroxybenzotriazole (67.9 mg), triethylamine (0.3 mL), 1-ethyl-3-(3-dimethylaminopropyl)carbodiimide hydrochloride (90.2 mg), tetrahydrofuran (0.7 mL) and N,N-dimethylformamide (0.7 mL), a similar reaction as in Example 110 was carried out to give the title compound (64.0 mg) as a yellow powder.

¹H-NMR (DMSO-d₆) δ: 1.12-1.34 (1H, m), 1.35-1.72 (3H, m), 2.64-2.74 (2H, m), 3.08-3.47 (7H, m), 4.37-4.48 (2H, m), 7.12-7.22 (3H, m), 7.25 (1H, d, J=8.8 Hz), 7.41-7.50 (1H, m), 7.56-7.70 (3H, m), 7.88-8.02 (3H, m), 9.12 (1H, s).

### Example 122

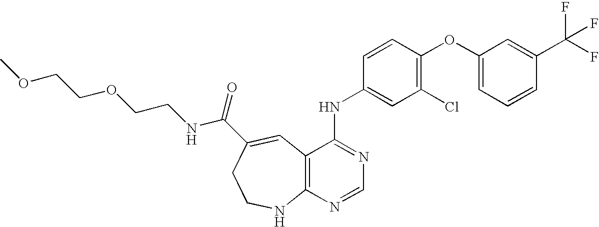

### Production of 4-({3-chloro-4-[3-(trifluoromethyl)phenoxy]phenyl}amino)-N-[2-(2-methoxyethoxy)ethyl]-8,9-dihydro-7H-pyrimido[4,5-b]azepine-6-carboxamide

Using 4-({3-chloro-4-[3-(trifluoromethyl)phenoxy]phenyl}amino)-8,9-dihydro-7H-pyrimido[4,5-b]azepine-6-carboxylic acid (130 mg), 2-(2-methoxyethoxy)ethylamine hydrochloride (68.3 mg), 1-hydroxybenzotriazole (58.4 mg), triethylamine (0.4 mL), 1-ethyl-3-(3-dimethylaminopropyl)carbodiimide hydrochloride (79.8 mg), tetrahydrofuran (0.7 mL) and N,N-dimethylformamide (0.7 mL), a similar reaction as in Example 110 was carried out to give the title compound (95.8 mg) as a white powder.

¹H-NMR (DMSO-d₆) δ: 2.65-2.73 (2H, m), 3.21 (3H, s), 3.28-3.39 (4H, m), 3.39-3.57 (6H, m), 7.14-7.23 (3H, m), 7.26 (1H, d, J=8.7 Hz), 7.46 (1H, d, J=7.6 Hz), 7.56-7.65 (2H, m), 7.68 (1H, t, J=4.4 Hz), 7.89 (1H, d, J=2.7 Hz), 7.98 (1H, s), 8.06 (1H, t, J=5.5 Hz), 9.15 (1H, s).

### Example 123

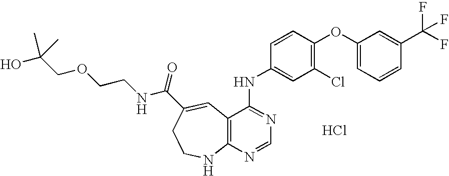

### Production of 4-({3-chloro-4-[3-(trifluoromethyl)phenoxy]phenyl}amino)-N-[2-(2-hydroxy-2-methylpropoxy)ethyl]-8,9-dihydro-7H-pyrimido[4,5-b]azepine-6-carboxamide hydrochloride

### (i) Production of 1-[2-(benzyloxy)ethoxy]-2-methyl-2-propanol

To a solution of 2-(benzyloxy)ethanol (4.6 mL) and 2,2-dimethyloxirane (8.30 g) in toluene (40 mL) was added 50% aqueous sodium hydroxide solution (8 mL), and the mixture was stirred at 100° C. for 28 hr. Water was added to the reaction mixture, and the mixture was extracted with ethyl acetate. The organic layer was washed with saturated brine, dried over anhydrous magnesium sulfate and concentrated under reduced pressure. The residue was dissolved in tetrahydrofuran (50 mL), and triethylamine (9 mL) and benzoyl chloride (3.8 mL) were successively added to the solution and the mixture was stirred at 0° C. for 2 hr. Water was added to the reaction mixture, and the mixture was extracted with ethyl acetate. The organic layer was washed with saturated brine, dried over anhydrous magnesium sulfate and concentrated under reduced pressure. The residue was separated and purified by silica gel column chromatography (eluent, hexane:ethyl acetate=80:20→33:67) to give the title compound (4.34 g) as a pale-orange oil.

¹H-NMR (CDCl₃) δ: 1.21 (6H, s), 2.60 (1H, br s), 3.33 (2H, s), 3.61-3.67 (2H, m), 3.68-3.74 (2H, m), 4.57 (2H, s), 7.26-7.41 (5H, m).

### (ii) Production of 1-(2-hydroxyethoxy)-2-methyl-2-propanol

To a solution of 1-[2-(benzyloxy)ethoxy]-2-methyl-2-propanol (4.34 g) in methanol (45 mL) was added 10% palladium-carbon (410 mg), and the mixture was stirred at room temperature for 5 days under hydrogen atmosphere. The mixture was filtrated through Celite to remove palladium-carbon, and the filtrate was concentrated under reduced pressure. The residue was separated and purified by silica gel column chromatography (eluent, hexane:ethyl acetate=50:50→0:100→ethyl acetate:methanol=95:5) to give the title compound (2.32 g) as a colorless oil.

¹H-NMR (CDCl₃) δ: 1.23 (6H, s), 1.71 (1H, br s), 2.49 (1H, br s), 3.34 (2H, s), 3.55-3.70 (2H, m), 3.70-3.85 (2H, m).

### (iii) Production of 2-(2-hydroxy-2-methylpropoxy)ethyl 4-methylbenzenesulfonate

To a solution of 1-(2-hydroxyethoxy)-2-methyl-2-propanol (1.12 g) in pyridine (10 mL) was added p-toluenesulfonyl chloride (1.93 g) under ice-cooling, and the mixture was stirred for 13 hr while raising the temperature to room temperature. Water was added to the reaction mixture and the mixture was extracted with ethyl acetate. The organic layer was washed with saturated brine, dried over anhydrous magnesium sulfate and concentrated under reduced pressure. The residue was separated and purified by silica gel column chromatography (eluent, hexane:ethyl acetate=80:20→33:67) to give the title compound (1.74 g) as a colorless oil.

¹H-NMR (CDCl₃) δ: 1.15 (6H, s), 2.14 (1H, br s), 2.45 (3H, s), 3.25 (2H, s), 3.66-3.73 (2H, m), 4.17-4.23 (2H, m), 7.35 (2H, br d, J=8 Hz), 7.81 (2H, br d, J=8 Hz).

### (iv) Production of N-formyl-N-[2-(2-hydroxy-2-methylpropoxy)ethyl]formamide

Using 2-(2-hydroxy-2-methylpropoxy)ethyl 4-methylbenzenesulfonate (1.73 g), sodium diformylimide (756 mg) and N,N-dimethylformamide (20 mL), a similar reaction as in Example 121 (iii) was carried out to give the title compound (162 mg) as a colorless oil.

¹H-NMR (CDCl₃) δ: 1.17 (6H, s), 2.27 (1H, s), 3.28 (2H, s), 3.66 (2H, t, J=5.4 Hz), 3.79-4.02 (2H, m), 8.88 (2H, s).

### (v) Production of 1-(2-aminoethoxy)-2-methyl-2-propanol hydrochloride

Using N-formyl-N-[2-(2-hydroxy-2-methylpropoxy)ethyl]formamide (162 mg), 6N hydrochloric acid (0.4 mL) and ethanol (5 mL), a similar reaction as in Example 121 (iv) was carried out to give the title compound (119 mg) as a white solid.

¹H-NMR (DMSO-d₆) δ: 1.09 (6H, s), 2.88-3.05 (2H, m), 3.21 (2H, s), 3.61 (2H, t, J=5.2 Hz), 8.02 (3H, br s).

### (vi) Production of 4-({3-chloro-4-[3-(trifluoromethyl)phenoxy]phenyl}amino)-N-[2-(2-hydroxy-2-methylpropoxy)ethyl]-8,9-dihydro-7H-pyrimido[4,5-b]azepine-6-carboxamide hydrochloride

Using 4-({3-chloro-4-[3-(trifluoromethyl)phenoxy]phenyl}amino)-8,9-dihydro-7H-pyrimido[4,5-b]azepine-6-carboxylic acid (150 mg), 1-(2-aminoethoxy)-2-methyl-2-propanol hydrochloride (119 mg), 1-hydroxybenzotriazole (128 mg), triethylamine (0.45 mL), 1-ethyl-3-(3-dimethylaminopropyl)carbodiimide hydrochloride (209 mg), tetrahydrofuran (0.7 mL) and N,N-dimethylformamide (0.7 mL), a similar reaction as in Example 110 was carried out to give 4-({3-chloro-4-[3-(trifluoromethyl)phenoxy]phenyl}amino)-N-[2-(2-hydroxy-2-methylpropoxy)ethyl]-8,9-dihydro-7H-pyrimido[4,5-b]azepine-6-carboxamide. To the solution of 4-({3-chloro-4-[3-(trifluoromethyl)phenoxy]phenyl}amino)-N-[2-(2-hydroxy-2-methylpropoxy)ethyl]-8,9-dihydro-7H-pyrimido[4,5-b]azepine-6-carboxamide in ethyl acetate (3 mL) was added 4N hydrochloric acid/ethyl acetate (0.1 mL). The mixture was stirred at room temperature for 0.5 hr and concentrated under reduced pressure. The residue was crystallized from diisopropyl ether/ethanol to give the title compound (88.8 mg) as a white powder.

¹H-NMR (DMSO-d₆) δ: 1.03 (6H, s), 2.68-2.81 (2H, m), 3.17 (2H, s), 3.28-3.40 (2H, m), 3.41-3.58 (4H, m), 7.15-7.28 (3H, m), 7.32 (1H, d, J=8.9 Hz), 7.45-7.58 (2H, m), 7.63 (1H, t, J=7.9 Hz), 7.82 (1H, d, J=2.6 Hz), 8.20 (1H, s), 8.41 (1H, t, J=5.4 Hz), 8.52 (1H, s), 10.17 (1H, s).

### Example 124

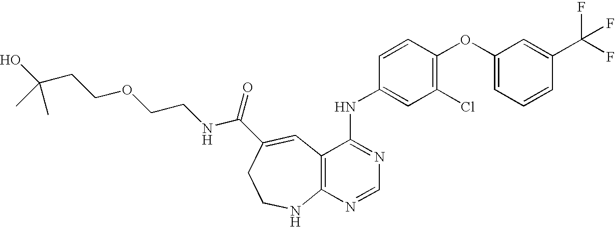

### Production of 4-({3-chloro-4-[3-(trifluoromethyl)phenoxy]phenyl}amino)-N-[2-(3-hydroxy-3-methylbutoxy)ethyl]-8,9-dihydro-7H-pyrimido[4,5-b]azepine-6-carboxamide

### (i) Production of 2-methyl-4-[2-(tetrahydro-2H-pyran-2-yloxy)ethoxy]butan-2-ol

To the solution of 2-(2-bromoethoxy)tetrahydro-2H-pyran (3 mL) and 3-methylbutane-1,3-diol (2.08 g) in toluene (40 mL) were added 50% aqueous sodium hydroxide solution (10 mL) and tetra-n-butylammonium hydrogen sulfate (673 mg), and the mixture was stirred at 40° C. for 17 hr. Water was added to the reaction mixture and the mixture was extracted with toluene. The organic layer was washed with saturated brine, dried over anhydrous magnesium sulfate and concentrated under reduced pressure. The residue was purified by silica gel column chromatography (eluent, hexane:ethyl acetate=90:/10→33/:67) to give the title compound (3.41 g) as a colorless oil.

¹H-NMR (CDCl₃) δ: 1.25 (6H, s), 1.44-1.91 (8H, m), 3.34 (1H, s), 3.46-3.68 (4H, m), 3.75 (2H, t, J=6 Hz), 3.80-3.94 (2H, m), 4.56-4.70 (1H, m).

### (ii) Production of 4-(2-hydroxyethoxy)-2-methylbutan-2-ol

2-Methyl-4-[2-(tetrahydro-2H-pyran-2-yloxy)ethoxy]butan-2-ol (3.40 g) was dissolved in methanol (40 mL). 4N Hydrochloric acid/ethyl acetate (4 mL) was added thereto and the mixture was stirred at room temperature for 2 hr. Additional 4N hydrochloric acid/ethyl acetate (1 mL) was added thereto, and the mixture was stirred for 1 hr. The reaction mixture was concentrated under reduced pressure, and the residue was separated and purified by silica gel column chromatography (eluent, hexane:ethyl acetate=50:50→0:100→ethyl acetate:methanol=95:5) to give the title compound (1.57 g) as a pale-yellow oil.

¹H-NMR (CDCl₃) δ: 1.26 (6H, s), 1.79 (2H, t, J=5.9 Hz), 3.50-3.66 (2H, m), 3.68-3.84 (4H, m).

### (iii) Production of 2-(3-hydroxy-3-methylbutoxy)ethyl 4-methylbenzenesulfonate

Using 4-(2-hydroxyethoxy)-2-methylbutan-2-ol (1.00 g), pyridine (6 mL) and p-toluenesulfonyl chloride (1.81 g), a similar reaction as in Example 123 (iii) was carried out to give the title compound (1.41 g) as a colorless oil.

¹H-NMR (CDCl₃) δ: 1.21 (6H, s), 1.7.2 (2H, t, J=5.9 Hz), 2.45 (3H, s), 2.61 (1H, s), 3.58-3.76 (4H, m), 4.06-4.26 (2H, m), 7.34 (2H, br d, J=8 Hz), 7.79 (2H, br d, J=8 Hz).

### (iv) Production of N-formyl-N-[2-(3-hydroxy-3-methylbutoxy)ethyl]formamide

Using 2-(3-hydroxy-3-methylbutoxy)ethyl 4-methylbenzenesulfonate (1.40 g), N,N-dimethylformamide (15 mL) and sodium diformylazanide (618 mg), a similar reaction as in Example 121 (iii) was carried out to give the title compound (305 mg) as a colorless oil.

¹H-NMR (CDCl₃) δ: 1.22 (6H, s), 1.73 (2H, t, J=6 Hz), 2.88 (1H, s), 3.60 (2H, t, J=5.4 Hz), 3.66 (2H, t, J=6 Hz), 3.79-4.01 (2H, m), 8.87 (2H, s).

### (v) Production of 4-(2-aminoethoxy)-2-methyl-2-butanol hydrochloride

Using N-formyl-N-[2-(3-hydroxy-3-methylbutoxy)ethyl]formamide (305 mg), 6N hydrochloric acid (1.3 mL) and ethanol (10 mL), a similar reaction as in Example 121 (iv) was carried out to give the title compound (265 mg) as a colorless oil.

¹H-NMR (DMSO-d₆) δ: 1.10 (6H, s), 1.61-1.78 (2H, m), 2.83-3.04 (2H, m), 3.46-3.68 (4H, m), 4.28 (1H, br s), 8.08 (3H, br s).

### (vi) Production of 4-({3-chloro-4-[3-(trifluoromethyl)phenoxy]phenyl}amino)-N-[2-(3-hydroxy-3-methylbutoxy)ethyl]-8,9-dihydro-7H-pyrimido[4,5-b]azepine-6-carboxamide

Using 4-({3-chloro-4-[3-(trifluoromethyl)phenoxy]phenyl}amino)-8,9-dihydro-7H-pyrimido[4,5-b]azepine-6-carboxylic acid (150 mg), 4-(2-aminoethoxy)-2-methyl-2-butanol hydrochloride (89.0 mg), 1-hydroxybenzotriazole (88.1 mg), triethylamine (0.45 mL), 1-ethyl-3-(3-dimethylaminopropyl)carbodiimide hydrochloride (121 mg), tetrahydrofuran (0.7 mL) and N,N-dimethylformamide (0.7 mL), a similar reaction as in Example 110 was carried out to give the title compound (142 mg) as a white powder.

¹H-NMR (DMSO-d₆) δ: 1.05 (6H, s), 1.61 (2H, t, J=7.2 Hz), 2.60-2.79 (2H, m), 3.22-3.39 (4H, m), 3.40-3.55 (4H, m), 4.17 (1H, s), 7.12-7.22 (3H, m), 7.26 (1H, d, J=8.9 Hz), 7.46 (1H, d, J=7.5 Hz), 7.56-7.64 (2H, m), 7.68 (1H, t, J=4.6 Hz), 7.89 (1H, d, J=2.6 Hz), 7.98 (1H, s), 8.03 (1H, t, J=5.4 Hz), 9.12 (1H, s).

### Example 125

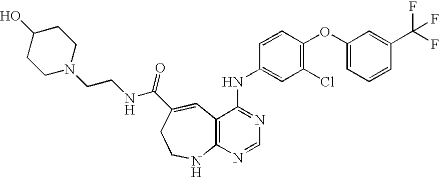

### Production of 4-({3-chloro-4-[3-(trifluoromethyl)phenoxy]phenyl}amino)-N-[2-(4-hydroxypiperidin-1-yl)ethyl]-8,9-dihydro-7H-pyrimido[4,5-b]azepine-6-carboxamide

Using 4-({3-chloro-4-[3-(trifluoromethyl)phenoxy]phenyl}amino)-8,9-dihydro-7H-pyrimido[4,5-b]azepine-6-carboxylic acid (129 mg), 1-(2-aminoethyl)-4-hydroxypiperidine (59.8 mg), 1-hydroxybenzotriazole (59.5 mg), triethylamine (0.1 mL), 1-ethyl-3-(3-dimethylaminopropyl)carbodiimide hydrochloride (82.4 mg), tetrahydrofuran (0.7 mL) and N,N-dimethyl formamide (0.7 mL), a similar reaction as in Example 110 was carried out to give the title compound (91.8 mg) as a white powder.

¹H-NMR (DMSO-d₆) δ: 1.24-1.45 (2H, m), 1.57-1.75 (2H, m), 2.04 (2H, t, J=10 Hz), 2.38 (2H, t, J=7.0 Hz), 2.60-2.78 (4H, m), 3.18-3.49 (5H, m), 4.49 (1H, d, J=4.2 Hz), 7.11-7.22 (3H, m), 7.25 (1H, d, J=8.7 Hz), 7.45 (1H, d, J=7.6 Hz), 7.54-7.64 (2H, m), 7.67 (1H, t, J=4.4 Hz), 7.84-7.94 (2H, m), 7.98 (1H, s), 9.12 (1H, s).

### Example 126

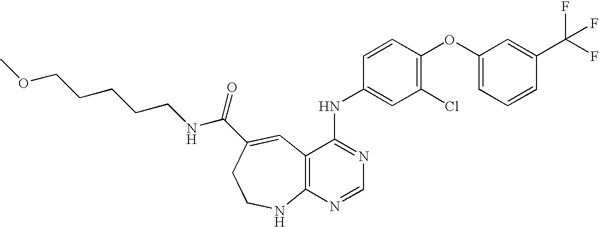

### Production of 4-({3-chloro-4-[3-(trifluoromethyl)phenoxy]phenyl}amino)-N-(5-methoxypentyl)-8,9-dihydro-7H-pyrimido[4,5-b]azepine-6-carboxamide

Using 4-({3-chloro-4-[3-(trifluoromethyl)phenoxy]phenyl}amino)-8,9-dihydro-7H-pyrimido[4,5-b]azepine-6-carboxylic acid (129 mg), 5-methoxy-1-pentylamine hydrochloride (60.0 mg), 1-hydroxybenzotriazole (55.2 mg), triethylamine (0.2 mL), 1-ethyl-3-(3-dimethylaminopropyl)carbodiimide hydrochloride (76.6 mg), tetrahydrofuran (0.7 mL) and N,N-dimethyl form/amide (0.7 mL), a similar reaction as in Example 110 was carried out to give the title compound (108 mg) as a white powder.

¹H-NMR (DMSO-d₆) δ: 1.24-1.40 (2H, m), 1.39-1.59 (4H, m), 2.62-2.76 (2H, m), 3.10-3.22 (2H, m), 3.18 (3H, s), 3.24-3.41 (4H, m), 7.11-7.22 (3H, m), 7.26 (1H, d, J=8.7 Hz), 7.46 (1H, d, J=8.0 Hz), 7.55-7.72 (3H, m), 7.90 (1H, d, J=2.7 Hz), 7.93-8.04 (2H, m), 9.14 (1H, s).

### Example 127

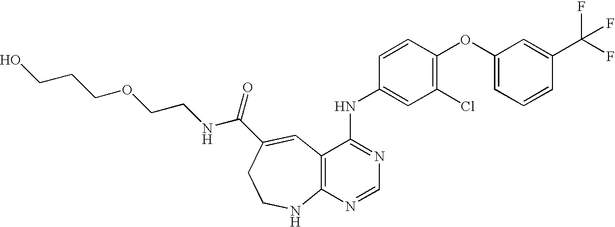

### Production of 4-({3-chloro-4-[3-(trifluoromethyl)phenoxy]phenyl}amino)-N-[2-(3-hydroxypropoxy)ethyl]-8,9-dihydro-7H-pyrimido[4,5-b]azepine-6-carboxamide

### (i) Production of 2-{2-[3-(benzyloxy)propoxy]ethoxy}tetrahydro-2H-pyran

Using 2-(2-bromoethoxy)tetrahydro-2H-pyran (8.00 g), 3-(benzyloxy)propan-1-ol (6.61 g), tetra-n-butylammonium hydrogen sulfate (1.31 g), toluene (80 mL) and 50% aqueous sodium hydroxide solution (20 mL), a similar reaction as in Example 124 (i) was carried out to give the title compound (6.19 g) as a yellow oil.

¹H-NMR (CDCl₃) δ: 1.42-2.00 (8H, m), 3.42-3.68 (8H, m), 3.77-3.94 (2H, m), 4.51 (2H, s), 4.58-4.71 (1H, m), 7.22-7.45 (5H, m).

### (ii) Production of 2-[3-(benzyloxy)propoxy]ethanol

To a solution of 2-{2-[3-(benzyloxy)propoxy]ethoxy}tetrahydro-2H-pyran (3.09 g) in methanol (30 mL) was added 6N hydrochloric acid (2.8 mL), and the mixture was stirred at room temperature for 31 hr. 1N aqueous sodium hydroxide solution was added to the reaction mixture and the mixture was concentrated under reduced pressure. The residue was extracted with ethyl acetate. The organic layer was washed with saturated brine, dried over anhydrous magnesium sulfate and concentrated under reduced pressure. The residue was separated and purified by silica gel column chromatography (eluent, hexane:ethyl acetate=90:10→33:67) to give the title compound (2.02 g) as a colorless oil.

¹H-NMR (CDCl₃) δ: 1.84-1.98 (2H, m), 2.01-2.13 (1H, m), 3.50-3.65 (6H, m), 3.66-3.79 (2H, m), 4.51 (2H, s), 7.15-7.53 (5H, m).

### (iii) Production of {[3-(2-azidoethoxy)propoxy]methyl}benzene

To a solution of 2-[3-(benzyloxy)propoxy]ethanol (2.00 g) in tetrahydrofuran (20 mL) was added 1,8-diazabicyclo[5,4,0]-7-undecene (3 mL) and diphenylphosphoryl azide (2.5 mL) under ice-cooling, and the mixture was stirred at room temperature for 0.5 hr. Water was added to the reaction mixture, and the mixture was extracted with ethyl acetate. The organic layer was washed with saturated brine, dried over anhydrous magnesium sulfate and concentrated under reduced pressure. The residue was separated and purified by silica gel column chromatography (eluent, hexane:ethyl acetate=90/10→50/50) to give 2-[3-(benzyloxy)propoxy]ethyl diphenyl phosphate. The obtained compound (4.10 g) was dissolved in N,N-dimethylformamide (50 mL). Sodium azide (906 mg) was added to the mixture and the mixture was stirred at 50° C. for 4 hr and at 80° C. for 24 hr. The reaction mixture was concentrated under reduced pressure. Water was added thereto and the mixture was extracted with ethyl acetate. The organic layer was washed with saturated brine, dried over anhydrous magnesium sulfate and concentrated under reduced pressure. The residue was separated and purified by silica gel column chromatography (eluent, hexane:ethyl acetate=95/5→67/33) to give the title compound (2.10 g) as a colorless oil.

¹H-NMR (CDCl₃) δ: 1.81-2.00 (2H, m), 3.26-3.41 (2H, m), 3.51-3.71 (6H, m), 4.51 (2H, s), 7.18-7.44 (5H, m).

### (iv) Production of tert-butyl {2-[3-(benzyloxy)propoxy]ethyl}carbamate

To a solution of {[3-(2-azidoethoxy)propoxy]methyl}benzene (2.10 g) in tetrahydrofuran (20 mL) were added water (2 mL) and triphenylphosphine (2.59 g), and the mixture was stirred at room temperature for 24 hr. The reaction mixture was concentrated under reduced pressure, and the residue was dissolved in methanol (30 mL). Triethylamine (3.5 mL) and di-tert-butyl dicarbonate (3 mL) were successively added thereto and the mixture was stirred at room temperature for 3 hr. The reaction mixture was concentrated under reduced pressure. Water was added to the residue and the mixture was extracted with ethyl acetate. The organic layer was dried over anhydrous magnesium sulfate and concentrated under reduced pressure. The residue was separated and purified by silica gel column chromatography (eluent, hexane:ethyl acetate=90:10→67:33) to give the title compound (2.70 g) as a colorless oil.

¹H-NMR (CDCl₃) δ: 1.44 (9H, s), 1.82-1.94 (2H, m), 3.23-3.34 (2H, m), 3.46 (2H, t, J=5.1 Hz), 3.50-3.59 (4H, m), 4.51 (2H, s), 4.88 (1H, br s), 7.18-7.41 (5H, m).

### (v) Production of tert-butyl [2-(3-hydroxypropoxy)ethyl]carbamate

Using tert-butyl {2-[3-(benzyloxy)propoxy]ethyl}carbamate (2.69 g), 10% palladium-carbon (308 mg) and methanol (30 mL), a similar reaction as in Example 123 (ii) was carried out to give the title compound (1.78 g) as a colorless oil.

¹H-NMR (CDCl₃) δ: 1.45 (9H, s), 1.76-1.91 (2H, m), 2.24 (1H, br s), 3.32 (2H, q, J=5 Hz), 3.51 (2H, t, J=5 Hz), 3.63 (2H, t, J=6 Hz), 3.77 (2H, t, J=6 Hz), 4.86 (1H, br s).

### (vi) Production of 3-(2-aminoethoxy)-1-propanol hydrochloride

tert-Butyl [2-(3-hydroxypropoxy)ethyl]carbamate (802 mg) was dissolved in ethanol (10 mL). 6N hydrochloric acid (3 mL) was added to the solution and the mixture was stirred at 50° C. for 22 hr. The reaction mixture was concentrated under reduced pressure to give the title compound (562 mg) as a colorless oil.

¹H-NMR (DMSO-d₆) δ: 1.54-1.78 (2H, m), 2.82-3.06 (2H, m), 3.21-3.65 (6H, m), 8.03 (3H, br s).

### (vii) Production of 4-({3-chloro-4-[3-(trifluoromethyl)phenoxy]phenyl}amino)-N-[2-(3-hydroxypropoxy)ethyl]-8,9-dihydro-7H-pyrimido[4,5-b]azepine-6-carboxamide

Using 4-({3-chloro-4-[3-(trifluoromethyl)phenoxy]phenyl}amino)-8,9-dihydro-7H-pyrimido[4,5-b]azepine-6-carboxylic acid (131 mg), 3-(2-aminoethoxy)-1-propanol hydrochloride (64.6 mg), 1-hydroxybenzotriazole (54.9 mg), triethylamine (0.4 mL), 1-ethyl-3-(3-dimethylaminopropyl)carbodiimide hydrochloride (81.2 mg), tetrahydrofuran (0.7 mL) and N,N-dimethylformamide (0.7 mL), a similar reaction as in Example 110 was carried out to give the title compound (115 mg) as a white powder.

¹H-NMR (DMSO-d₆) δ: 1.56-1.70 (2H, m), 2.64-2.72 (2H, m), 3.27-3.39 (4H, m), 3.39-3.50 (6H, m), 4.37 (1H, t, J=355.1 Hz), 7.13-7.22 (3H, m), 7.26 (1H, d, J=8.7 Hz), 7.46 (1H, d, J=8.0 Hz), 7.55-7.65 (2H, m), 7.68 (1H, t, J=4.5 Hz), 7.90 (1H, d, J=2.7 Hz), 7.98 (1H, s), 8.03 (1H, t, J=5.5 Hz), 9.14 (1H, s).

### Example 128

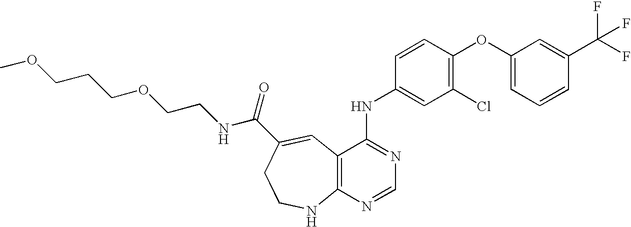

### Production of 4-({3-chloro-4-[3-(trifluoromethyl)phenoxy]phenyl}amino)-N-[2-(3-methoxypropoxy)ethyl]-8,9-dihydro-7H-pyrimido[4,5-b]azepine-6-carboxamide

### (i) Production of tert-butyl [2-(3-methoxypropoxy)ethyl]carbamate

To a suspension of 60% sodium hydride (dispersion in mineral oil, 220 mg) in tetrahydrofuran (30 mL) was added a solution of tert-butyl [2-(3-hydroxypropoxy)ethyl]carbamate (948 mg) in tetrahydrofuran (10 mL) under ice-cooling, and the mixture was stirred at 0° C. for 0.5 hr. Methyl iodide (0.3 mL) was added dropwise to the reaction solution, and the mixture was stirred at room temperature for 18 hr. To the reaction mixture was added saturated aqueous ammonium chloride solution, and the mixture was extracted with ethyl acetate. The organic layer was washed with saturated brine, dried over anhydrous magnesium sulfate and concentrated under reduced pressure. The residue was separated and purified by silica gel column chromatography (eluent, hexane:ethyl acetate=95:5→50:50) to give the title compound (631 mg) as a colorless oil.

¹H-NMR (CDCl₃) δ: 1.45 (9H, s), 1.76-1.94 (2H, m), 3.20-3.40 (2H, m), 3.34 (3H, s), 3.40-3.61 (6H, m), 4.94 (1H, br s).

### (ii) Production of 2-(3-methoxypropoxy)ethylamine hydrochloride

Using tert-butyl [2-(3-methoxypropoxy)ethyl]carbamate (625 mg), 6N hydrochloric acid (2 mL) and ethanol (5 mL), a similar reaction as in Example 127 (vi) was carried out to give the title compound (457 mg) as a colorless oil.

¹H-NMR (DMSO-d₆) δ: 1.68-1.83 (2H, m), 2.94 (2H, t, J=5.5 Hz), 3.22 (3H, s), 3.38 (2H, t, J=6.4 Hz), 3.47 (2H, t, J=6.4 Hz), 3.55 (2H, t, J=5.5 Hz), 8.03 (3H, br s).

### (iii) Production of 4-({3-chloro-4-[3-(trifluoromethyl)phenoxy]phenyl}amino)-N-[2-(3-methoxypropoxy)ethyl]-8,9-dihydro-7H-pyrimido[4,5-b]azepine-6-carboxamide

Using 4-({3-chloro-4-[3-(trifluoromethyl)phenoxy]phenyl}amino)-8,9-dihydro-7H-pyrimido[4,5-b]azepine-6-carboxylic acid (130 mg), 2-(3-methoxypropoxy)ethylamine hydrochloride (69.8 mg), 1-hydroxybenzotriazole (56.6 mg), triethylamine (0.4 mL), 1-ethyl-3-(3-dimethylaminopropyl) carbodiimide hydrochloride (79.7 mg), tetrahydrofuran (0.7 mL) and N,N-dimethylformamide (0.7 mL), a similar reaction as in Example 110 was carried out to give the title compound (123 mg) as a white powder.

¹H-NMR (DMSO-d₆) δ: 1.64-1.77 (2H, m), 2.63-2.74 (2H, m), 3.16 (3H, s), 3.27-3.39 (6H, m), 3.39-3.50 (4H, m), 7.12-7.22 (3H, m), 7.26 (1H, d, J=8.7 Hz), 7.46 (1H, d, J=7.6 Hz), 7.55-7.65 (2H, m), 7.68 (1H, t, J=4.5 Hz), 7.89 (1H, d, J=2.7 Hz), 7.98 (1H, s), 8.04 (1H, t, J=5.5 Hz), 9.14 (1H, s).

### Exammple 129

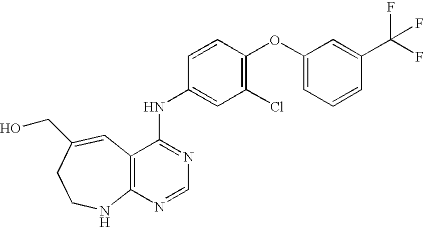

### Production of [4-({3-chloro-4-[3-(trifluoromethyl)phenoxy]phenyl}amino)-8,9-dihydro-7H-pyrimido[4,5-b]azepin-6-yl]methanol

A solution of 4-({3-chloro-4-[3-(trifluoromethyl)phenoxy]phenyl}amino)-8,9-dihydro-7H-pyrimido[4,5-b]azepine-6-carboxylic acid (400 mg) in tetrahydrofuran (10 mL) was cooled to −10° C. Triethylamine (0.175 mL) and isobutyl chloroformate (0.12 mL) were added to the solution and the mixture was stirred at −10° C. for 15 min. A solution of sodium borohydride (127 mg) in methanol (2 mL) was added dropwise to the reaction solution, and the mixture was stirred at −10° C. for 30 min. Water was added to the reaction mixture, and the mixture was extracted with ethyl acetate. The organic layer was dried over anhydrous magnesium sulfate and concentrated under reduced pressure. The residue was separated and purified by silica gel column chromatography (eluent, hexane:ethyl acetate=50:50→0:100), and crystallized from diisopropyl ether-ethyl acetate to give the title compound (220 mg) as colorless crystals.

¹H-NMR (DMSO-d₆) δ: 2.39-2.47 (2H, m), 3.28-3.37 (2H, m), 4.03 (2H, d, J=5 Hz), 4.86 (1H, t, J=5 Hz), 6.47 (1H, s), 7.14-7.20 (2H, m), 7.22 (1H, d, J=8.8 Hz), 7.32 (1H, t, J=4.5 Hz), 7.45 (1H, d, J=8.0 Hz), 7.55-7.64 (2H, m), 7.88 (1H, d, J=2.5 Hz), 7.93 (1H, s), 8.79 (1H, s).

### Example 130

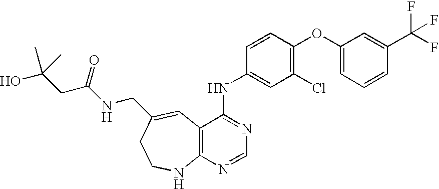

### Production of N-{[4-({3-chloro-4-[3-(trifluoromethyl)phenoxy]phenyl}amino)-8,9-dihydro-7H-pyrimido[4,5-b]azepin-6-yl]methyl}-3-hydroxy-3-methylbutanamide

### (i) Production of 6-(azidomethyl)-N-{3-chloro-4-[3-(trifluoromethyl)phenoxy]phenyl}-8,9-dihydro-7H-pyrimido[4,5-b]azepin-4-amine

To the solution of [4-({3-chloro-4-[3-(trifluoromethyl)phenoxy]phenyl}amino)-8,9-dihydro-7H-pyrimido[4,5-b]azepin-6-yl]methanol (103 mg) in tetrahydrofuran (1 mL) were added diazabicyclo[5,4,0]-7-undecene (0.1 mL) and diphenylphosphoryl azide (0.07 mL), and the mixture was stirred at 40° C. for 0.5 hr. Water was added to the reaction mixture and the mixture was extracted with ethyl acetate. The organic layer was washed with saturated brine, dried over anhydrous magnesium sulfate and concentrated under reduced pressure. The residue was separated and purified by silica gel column chromatography (eluent, hexane:ethyl acetate=67:33→0:100) and crystallized from hexane-diisopropyl ether to give the title compound (80 mg) as pale-orange crystals.

¹H-NMR (CDCl₃) δ: 2.57-2.64 (2H, m), 3.51-3.59 (2H, m), 3.95 (2H, s), 5.62 (1H, t, J=4.1 Hz), 6.29 (1H, s), 6.51 (1H, s), 7.05 (1H, d, J=8.9 Hz), 7.10 (1H, dd, J= 8 Hz, 2.1 Hz), 7.17-7.21 (1H, m), 7.29-7.38 (2H, m), 7.42 (1H, t, J=8 Hz), 7.72 (1H, d, J=2.6 Hz), 8.13 (1H, s).

### (ii) Production of N-{[4-({3-chloro-4-[3-(trifluoromethyl)phenoxy]phenyl}amino)-8,9-dihydro-7H-pyrimido[4,5-b]azepin-6-yl]methyl}-3-hydroxy-3-methylbutanamide

To a solution of 6-(azidomethyl)-N-{3-chloro-4-[3-(trifluoromethyl)phenoxy]phenyl}-8,9-dihydro-7H-pyrimido[4,5-b]azepin-4-amine (70.1 mg) in tetrahydrofuran (0.6 mL) and water (0.06 mL) was added triphenylphosphine (42.2 mg), and the mixture was stirred at room temperature for 24 hr. The reaction mixture was concentrated under reduced pressure, and the residue was separated and purified by basic silica gel column chromatography (eluent, hexane:ethyl acetate=10:90→0:100→ethyl acetate:methanol=90:10). The purified substance was dissolved in ethyl acetate (1 mL). 4N Hydrochloric acid/ethyl acetate (0.1 mL) was added to the solution, and the mixture was stirred for 1 hr. The reaction mixture was concentrated under reduced pressure to give 6-(aminomethyl)-N-{3-chloro-4-[3-(trifluoromethyl)phenoxy]phenyl}-8,9-dihydro-7H-pyrimido[4,5-b]azepin-4-amine dihydrochloride. Using the obtained compound (76.8 mg), 3-hydroxy-3-methylbutanoic acid (27.8 mg), 1-hydroxybenzotriazole (30.4 mg), triethylamine (0.2 mL), 1-ethyl-3-(3-dimethylaminopropyl)carbodiimide hydrochloride (44.2 mg), tetrahydrofuran (0.4 mL) and N,N-dimethylformamide (0.4 mL), a similar reaction as in Example 110 was carried out to give the title compound (38.3 mg) as a white powder.

¹H-NMR (DMSO-d₆) δ: 1.15 (6H, s), 2.26 (2H, s), 2.35-2.44 (2H, m), 3.27-3.37 (2H, m), 3.88 (2H, d, J=5.5 Hz), 4.87 (1H, s), 6.44 (1H, s), 7.15-7.21 (2H, m), 7.24 (1H, d, J=8.9 Hz), 7.35 (1H, t, J=4.6 Hz), 7.45 (1H, d, J=7.7 Hz), 7.56-7.66 (2H, m), 7.88 (1H, d, J=2.5 Hz), 7.93 (1H, s), 7.98 (1H, t, J=5.5 Hz), 8.77 (1H, s).

### Example 131

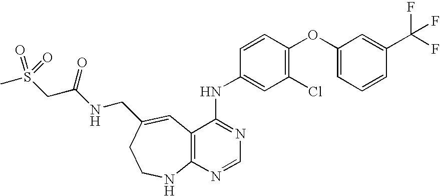

### Production of N-{[4-({3-chloro-4-[3-(trifluoromethyl)phenoxy]phenyl}amino)-8,9-dihydro-7H-pyrimido[4,5-b]azepin-6-yl]methyl}-2-(methylsulfonyl)acetamide

### (i) Production of 6-(aminomethyl)-N-{3-chloro-4-[3-(trifluoromethyl)phenoxy]phenyl}-8,9-dihydro-7H-pyrimido[4,5-b]azepin-4-amine dihydrochloride

Using 6-(azidomethyl)-N-{3-chloro-4-[3-(trifluoromethyl)phenoxy]phenyl}-8,9-dihydro-7H-pyrimido[4,5-b]azepin-4-amine (65.3 mg), triphenylphosphine (39.8 mg), tetrahydrofuran (0.6 mL), water (0.06 mL), di-tert-butyl dicarbonate (0.1 mL), triethylamine (0.1 mL) and methanol (1 mL), a similar reaction as in Example 127 (iv) was carried out to give tert-butyl {[4-({3-chloro-4-[3-(trifluoromethyl)phenoxy]phenyl}amino)-8,9-dihydro-7H-pyrimido[4,5-b]azepin-6-yl]methyl}carbamate. Using the obtained compound (73.8 mg), ethanol (2 mL) and 6N hydrochloric acid (0.15 mL), a similar reaction as in Example 127 (vi) was carried out to give the title compound (46.1 mg) as a white powder.

¹H-NMR (DMSO-d₆) δ: 2.53-2.62 (2H, m), 3.45 (2H, br s), 3.57-3.71 (2H, m), 6.74 (1H, s), 7.16-7.27 (2H, m), 7.31 (1H, d, J=9.1 Hz), 7.49 (1H, d, J=8.0 Hz), 7.57-7.70 (2H, m), 7.91 (1H, d, J=2.7 Hz), 8.20 (1H, s), 8.26 (1H, br s), 8.38 (3H, br s), 9.87 (1H, br s).

### (ii) Production of N-{[4-({3-chloro-4-[3-(trifluoromethyl)phenoxy]phenyl}amino)-8,9-dihydro-7H-pyrimido[4,5-b]azepin-6-yl]methyl}-2-(methylsulfonyl)acetamide

Using 6-(aminomethyl)-N-{3-chloro-4-[3-(trifluoromethyl)phenoxy]phenyl}-8,9-dihydro-7H-pyrimido[4,5-b]azepin-4-amine dihydrochloride (40.0 mg), (methylsulfonyl)acetic acid (17.4 mg), 1-hydroxybenzotriazole (21.7 mg), triethylamine (0.1 mL), 1-ethyl-3-(3-dimethylaminopropyl)carbodiimide hydrochloride (31.6 mg), tetrahydrofuran (0.3 mL) and N,N-dimethylformamide (0.3 mL), a similar reaction as in Example 110 was carried out to give the title compound (19.2 mg) as a white powder.

¹H-NMR (DMSO-d₆) δ: 2.36-2.45 (2H, m), 3.12 (3H, s), 3.26-3.38 (2H, m), 3.92 (2H, d, J=5.7 Hz), 4.12 (2H, s), 6.49 (1H, s), 7.15-7.21 (2H, m), 7.24 (1H, d, J=8.7 Hz), 7.36 (1H, t, J=4.7 Hz), 7.45 (1H, d, J=7.6 Hz), 7.55-7.67 (2H, m), 7.91 (1H, d, J=2.7 Hz), 7.94 (1H, s), 8.54 (1H, t, J=5.7 Hz), 8.72 (1H, s).

### Example 132

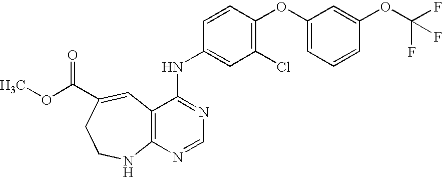

### Production of methyl 4-({3-chloro-4-[3-(trifluoromethoxy)phenoxy]phenyl}amino)-8,9-dihydro-7H-pyrimido[4,5-b]azepine-6-carboxylate

### (i) Production of methyl 4-[[6-({3-chloro-4-[3-(trifluoromethoxy)phenoxy]phenyl}amino)-5-formylpyrimidin-4-yl](4-methoxybenzyl)amino]butanoate

To the solution of 4,6-dichloro-5-formylpyrimidine (0.50 g) in acetonitrile (5.0 mL) were added potassium phosphate (0.60 g) and 3-chloro-4-[3-(trifluoromethoxy)phenoxy]aniline (0.858 g) at room temperature. The mixture was stirred at room temperature for 16 hr, and ethyl acetate was added to the reaction mixture. The mixture was washed with water and saturated brine, dried over anhydrous magnesium sulfate, and concentrated under reduced pressure. To the solution of the residue in N,N-dimethylformamide (15 mL) were added methyl 4-[(4-methoxybenzyl)amino]butanoate hydrochloride (0.775 g) and sodium carbonate (0.30 g) at room temperature. The mixture was stirred at room temperature for 20 hr. Water was added to the reaction mixture and the mixture was extracted with ethyl acetate. The organic layer was washed with water and saturated brine, dried over anhydrous magnesium sulfate and concentrated under reduced pressure. The residue was separated and purified by column chromatography (eluent, hexane:ethyl acetate=9:1→1:1) to give the title compound (0.32 g) as yellow amorphous.

¹H-NMR (CDCl₃) δ: 1.97-2.10 (2H, m), 2.33 (2H, t, J=7.2 Hz), 3.51-3.61 (2H, m), 3.66 (3H, s), 3.82 (3H, s), 4.80 (2H, s), 6.79-6.95 (5H, m), 7.07 (1H, d, J=8.7 Hz), 7.17 (2H, d, J=9.0 Hz), 7.29-7.34 (1H, m), 7.53 (1H, dd, J=8.7, 2.7 Hz), 8.02 (1H, d, J=2.7 Hz), 8.33 (1H, s), 9.81 (1H, s), 11.22 (1H, s).

### (ii) Production of methyl 4-({3-chloro-4-[3-(trifluoromethoxy)phenoxy]phenyl}amino)-9-(4-methoxybenzyl)-8,9-dihydro-7H-pyrimido[4,5-b]azepine-6-carboxylate

To the solution of methyl 4-[[6-({3-chloro-4-[3-(trifluoromethoxy)phenoxy]phenyl}amino)-5-formylpyrimidin-4-yl](4-methoxybenzyl)amino]butanoate (0.32 g) in dimethyl carbonate (6.0 mL) was added 28% sodium methoxide-methanol solution (0.28 g) at room temperature. The mixture was stirred at room temperature for 4 days. Ethyl acetate was added to the mixture. The mixture was washed with water and saturated brine, dried over anhydrous magnesium sulfate and concentrated under reduced pressure. The residue was separated and purified by column chromatography (eluent, hexane:ethyl acetate=9:1→3:2) to give the title compound (273 mg) as pale-yellow amorphous.

¹H-NMR (CDCl₃) δ: 2.67 (2H, d, J=4.8 Hz), 3.36 (2H, d, J=4.8 Hz), 3.78 (3H, s), 3.81 (3H, s), 4.87 (2H, s), 6.78-6.96 (6H, m), 7.07 (1H, d, J=8.7 Hz), 7.23 (2H, d, J=8.4 Hz), 7.29-7.35 (1H, m), 7.39 (1H, dd, J=8.7, 2.4 Hz), 7.68 (1H, s), 7.74 (1H, d, J=2.4 Hz), 8.24 (1H, s).

### (iii) Production of methyl 4-({3-chloro-4-[3-(trifluoromethoxy)phenoxy]phenyl}amino)-8,9-dihydro-7H-pyrimido[4,5-b]azepine-6-carboxylate

To the solution of methyl 4-({3-chloro-4-[3-(trifluoromethoxy)phenoxy]phenyl}amino)-9-(4-methoxybenzyl)-8,9-dihydro-7H-pyrimido[4,5-b]azepine-6-carboxylate (251 mg) in 1,2-dichloroethane (3.0 mL) was added trifluoroacetic acid (3.0 mL) at room temperature. The mixture was stirred at 70° C. for 18 hr and concentrated under reduced pressure. The residue was basified by adding aqueous sodium bicarbonate solution and extracted with ethyl acetate. The organic layer was washed with saturated brine, dried over anhydrous magnesium sulfate and concentrated under reduced pressure. The residue was separated and purified by column chromatography (eluent, hexane:ethyl acetate=4:1→1:1) and crystallized from ethyl acetate-diisopropyl ether to give the title compound (84 mg) as colorless crystals.

¹H-NMR (CDCl₃) δ: 2.91 (2H, t, J=4.8 Hz), 3.53 (2H, q, J=4.8 Hz), 3.82 (3H, s), 5.79-5.87 (1H, m), 6.82-6.88 (3H, m), 6.90-6.96 (1H, m), 7.07 (1H, d, J=8.7 Hz), 7.29-7.35 (1H, m), 7.38 (1H, dd, J=8.7, 2.7 Hz), 7.67 (1H, s), 7.74 (1H, d, J=2.7 Hz), 8.13 (1H, s).

### Example 133

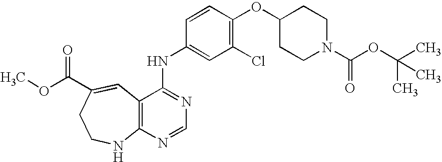

### Production of methyl 4-[(4-{[1-(tert-butoxycarbonyl)piperidin-4-yl]oxy}-3-chlorophenyl)amino]-8,9-dihydro-7H-pyrimido[4,5-b]azepine-6-carboxylate

To the solution of methyl 4-[(4-{[1-(tert-butoxycarbonyl)piperidin-4-yl]oxy}-3-chlorophenyl)amino]-9-(4-methoxybenzyl)-8,9-dihydro-7H-pyrimido[4,5-b]azepine-6-carboxylate (268 mg) in 1,2-dichloroethane (3.0 mL) was added trifluoroacetic acid (3.0 mL) at room temperature, and the mixture was stirred at 70° C. for 18 hr. The mixture was concentrated under reduced pressure, and tetrahydrofuran (10 mL) and triethylamine (0.49 mL) were added to the residue. Di-tert-butyl dicarbonate (0.096 mL) was additionally added to the residue at room temperature. The mixture was stirred at room temperature for 2 hr. Water was added thereto and the mixture was extracted with ethyl acetate. The organic layer was washed with saturated brine, dried over anhydrous magnesium sulfate and concentrated under reduced pressure. The residue was separated and purified by column chromatography (eluent, hexane:ethyl acetate=1:1→ethyl acetate) to give the title compound (149 mg) as colorless crystals.

¹H-NMR (CDCl₃) δ: 1.47 (9H, s), 1.75-1.96 (4H, m), 2.89 (2H, t, J=4.5 Hz), 3.36-3.46 (2H, m), 3.49-3.54 (2H, m), 3.64-3.72 (2H, m), 3.81 (3H, s), 4.43-4.51 (1H, m), 5.72-5.80 (1H, m), 6.74 (1H, s), 6.95 (1H, d, J=9.0 Hz), 7.28 (1H, dd, J=9.0, 2.7 Hz), 7.55 (1H, d, J=2.7 Hz), 7.66 (1H, s), 8.08 (1H, s).

### Example 134

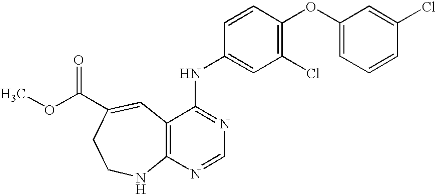

### Production of methyl 4-{[3-chloro-4-(3-chlorophenoxy)phenyl]amino}-8,9-dihydro-7H-pyrimido[4,5-b]azepine-6-carboxylate

### (i) Production of 4-chloro-6-{[3-chloro-4-(3-chlorophenoxy)phenyl]amino}pyrimidine-5-carbaldehyde

To a solution of 4,6-dichloro-5-formylpyrimidine (0.50 g) in tetrahydrofuran (10 mL) were added triethylamine (0.39 mL) and 3-chloro-4-(3-chlorophenoxy)aniline (0.718 g) at room temperature. The mixture was stirred at room temperature for 16 hr, and ethyl acetate was added thereto. The mixture was washed with water and saturated brine, dried over anhydrous magnesium sulfate and concentrated under reduced pressure to give the title compound (968 mg) as yellow crystals.

¹H-NMR (CDCl₃) δ: 6.84-6.88 (1H, m), 6.94-6.96 (1H, m), 7.05-7.11 (2H, m), 7.23-7.29 (1H, m), 7.50 (1H, dd, J=8.7, 2.7 Hz), 7.97 (1H, d, J=2.7 Hz), 8.59 (1H, s), 10.47 (1H, s), 11.14 (1H, s).

### (ii) Production of methyl 4-[(6-{[3-chloro-4-(3-chlorophenoxy)phenyl]amino}-5-formylpyrimidin-4-yl)(4-methoxybenzyl)amino]butanoate

To the mixture of 4-chloro-6-{[3-chloro-4-(3-chlorophenoxy)phenyl]amino}pyrimidine-5-carbaldehyde (968 mg) and sodium carbonate (390 mg) in N,N-dimethylformamide (5.0 mL) was added a mixture of methyl 4-[(4-methoxybenzyl)amino]butanoate hydrochloride (671 mg) and triethylamine (0.38 mL) in N,N-dimethylformamide. The mixture was stirred at room temperature for 20 hr. Water was added to the mixture, and the mixture was extracted with ethyl acetate. The organic layer was washed with water and saturated brine, dried over anhydrous magnesium sulfate and concentrated under reduced pressure. The residue was separated and purified by column chromatography (eluent, hexane:ethyl acetate=4:1→3:2) to give the title compound (431 mg) as yellow amorphous.

¹H-NMR (CDCl₃) δ: 1.99-2.10 (2H, m), 2.33 (2H, t, J=7.4 Hz), 3.57 (2H, t, J=7.5 Hz), 3.65 (3H, s), 3.81 (3H, s), 4.79 (2H, s), 6.83-6.94 (4H, m), 7.03-7.07 (2H, m), 7.17 (2H, d, J=9.0 Hz), 7.22 (1H, d, J=8.4 Hz), 7.51 (1H, dd, J=8.7, 2.7 Hz), 7.99 (1H, d, J=2.7 Hz), 8.32 (1H, s), 9.81 (1H, s), 11.20 (1H, s).

### (iii) Production of methyl 4-{[3-chloro-4-(3-chlorophenoxy)phenyl]amino}-9-(4-methoxybenzyl)-8,9-dihydro-7H-pyrimido[4,5-b]azepine-6-carboxylate

Using methyl 4-[(6-{[3-chloro-4-(3-chlorophenoxy)phenyl]amino}-5-formylpyrimidin-4-yl)(4-methoxybenzyl)amino]butanoate (431 mg), a solution of sodium methoxide in methanol (28%, 0.39 g) and dimethyl carbonate (4.0 mL), a similar reaction as in Example 132 (ii) was carried out to give the title compound (312 mg) as yellow amorphous.

¹H-NMR (CDCl₃) δ: 2.66 (2H, t, J=4.8 Hz), 3.35 (2H, t, J=4.8 Hz), 3.78 (3H, s), 3.81 (3H, s), 4.87 (2H, s), 6.83-6.90 (4H, m), 6.94 (1H, t, J=2.1 Hz), 7.06 (2H, d, J=8.4 Hz), 7.21-7.27 (3H, m), 7.38 (1H, dd, J=8.7, 2.4 Hz), 7.68 (1H, s), 7.73 (1H, d, J=2.4 Hz), 8.24 (1H, s).

### (iv) Production of methyl 4-{[3-chloro-4-(3-chlorophenoxy)phenyl]amino}-8,9-dihydro-7H-pyrimido[4,5-b]azepine-6-carboxylate

Using methyl 4-{[3-chloro-4-(3-chlorophenoxy)phenyl]amino}-9-(4-methoxybenzyl)-8,9-dihydro-7H-pyrimido[4,5-b]azepine-6-carboxylate (312 mg), trifluoroacetic acid (3.0 mL) and 1,2-dichloroethane (3.0 mL), a similar reaction as in Example 132 (iii) was carried out to give the title compound (203 mg) as colorless crystals.

¹H-NMR (CDCl₃) δ: 2.89-2.92 (2H, m), 3.50-3.56 (2H, m), 3.82 (3H, s), 5.78-5.86 (1H, m), 6.82-6.89 (2H, m), 6.93-6.95 (1H, m), 7.04-7.07 (2H, m), 7.22 (1H, d, J=8.7 Hz), 7.37 (1H, dd, J=8.7, 2.4 Hz), 7.67 (1H, s), 7.73 (1H, d, J=2.4 Hz), 8.12 (1H, s).

### Example 135

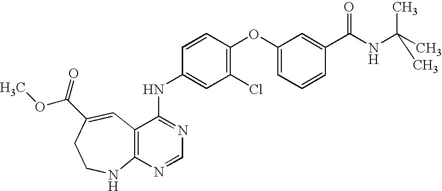

### Production of methyl 4-[(4-{3-[(tert-butylamino) carbonyl]phenoxy}-3-chlorophenyl)amino]-8,9-dihydro-7H-pyrimido[4,5-b]azepine-6-carboxylate

### (i) Production of 3-(2-chloro-4-nitrophenoxy)benzoic acid

To a solution of methyl 3-(2-chloro-4-nitrophenoxy)benzoate (4.81 g) in ethanol (30 mL) and tetrahydrofuran (20 mL) was added 1N aqueous sodium hydroxide solution (19.1 mL) at room temperature. The mixture was stirred at room temperature for 3 days, and concentrated under reduced pressure. The residue was acidified by adding 1N hydrochloric acid and extracted with ethyl acetate. The organic layer was washed with saturated brine, dried over anhydrous magnesium sulfate, and concentrated under reduced pressure. The precipitated crystals were collected by filtration. The crystals were washed with diisopropyl ether and hexane to give the title compound (4.28 g) as colorless crystals.

¹H-NMR (CDCl₃) δ: 6.94 (1H, d, J=9.0 Hz), 7.33-7.37 (1H, m), 7.54-7.59 (1H, m), 7.76-7.78 (1H, m), 7.98-8.02 (1H, m), 8.10 (1H, dd, J=9.0, 2.7 Hz), 8.41 (1H, d, J=2.7 Hz).

### (ii) Production of N-(tert-butyl)-3-(2-chloro-4-nitrophenoxy)benzamide

To the solution of 3-(2-chloro-4-nitrophenoxy)benzoic acid (4.0 g) and N,N-dimethylformamide (0.2 mL) in tetrahydrofuran (60 mL) was added thionyl chloride (1.49 mL) at room temperature. The mixture was stirred at room temperature for 1 hr and concentrated under reduced pressure. A solution of the residue in tetrahydrofuran (20 mL) was added dropwise to a solution of tert-butylamine (1.30 g) and triethylamine (2.85 mL) in tetrahydrofuran (30 mL) at 0° C. The mixture was stirred at room temperature for 14 hr. Aqueous sodium bicarbonate solution was added to the mixture, and the mixture was extracted with ethyl acetate. The organic layer was washed with saturated brine, dried over anhydrous magnesium sulfate and concentrated under reduced pressure to give the title compound (4.43 g) as pale-yellow amorphous.

¹H-NMR (CDCl₃) δ: 1.47 (9H, s), 5.92 (1H, br s), 6.91 (1H, d, J=9.0 Hz), 7.15-7.22 (1H, m), 7.44-7.57 (3H, m), 8.07 (1H, dd, J=9.0, 2.7 Hz), 8.39 (1H, t, J=2.7 Hz).

### (iii) Production of 3-(4-amino-2-chlorophenoxy)-N-(tert-butyl)benzamide

A mixture of N-(tert-butyl)-3-(2-chloro-4-nitrophenoxy)benzamide (4.43 g), reduced iron (3.80 g) and calcium chloride (0.76 g) in 15% water-containing ethanol (130 mL) was heated under reflux for 14 hr. The insoluble material was removed by filtration and the filtrate was concentrated under reduced pressure. Water was added to the residue, and the mixture was extracted with ethyl acetate. The organic layer was washed with water and saturated brine, dried over anhydrous magnesium sulfate and concentrated under reduced pressure. The residue was separated and purified by column chromatography (eluent, hexane:ethyl acetate=9:1→1:1) to give the title compound (2.60 g) as orange amorphous.

¹H-NMR (CDCl₃) δ: 1.45 (9H, s), 3.70 (2H, br s), 5.91 (1H, br s), 6.57 (1H, dd, J=8.4, 2.7 Hz), 6.78 (1H, d, J=8.4 Hz), 6.90 (1H, d, J=8.4 Hz), 6.92-7.01 (1H, m), 7.24-7.33 (3H, m).

### (iv) Production of tert-butyl 4-[(6-chloro-5-formylpyrimidin-4-yl)(4-methoxybenzyl)amino]butanoate

Using 4,6-dichloro-5-formylpyrimidine (365 mg), tert-butyl 4-[(4-methoxybenzyl)amino]butanate (613 mg), potassium phosphate (0.87 g) and acetonitrile (36 mL), a similar reaction as in Example 1 (i) was carried out to give the title compound (492 mg) as a pale-yellow oil.

¹H-NMR (CDCl₃) δ: 1.42 (9H, s), 1.84-1.96 (2H, m), 2.21 (2H, t, J=7.2 Hz), 3.63 (2H, t, J=7.2 Hz), 3.79 (3H, s), 4.58 (2H, s), 6.82 (2H, d, J=8.7 Hz), 7.00 (2H, d, J=8.7 Hz), 8.38 (1H, s), 10.18 (1H, s).

### (v) Production of tert-butyl-4-[{6-[(4-{3-[(tert-butylamino)carbonyl]phenoxy}-3-chlorophenyl)amino]-5-formylpyrimidin-4-yl}(4-methoxybenzyl)amino]butanoate

A mixture of tert-butyl 4-[(6-chloro-5-formylpyrimidin-4-yl)(4-methoxybenzyl)amino]butanoate (300 mg), 3-(4-amino-2-chlorophenoxy)-N-(tert-butyl)benzamide (228 mg) and sodium carbonate (76 mg) in N,N-dimethylformamide (3.0 mL) was stirred at 40° C. for 4 days. Water was added to reaction mixture, and the mixture was extracted with ethyl acetate. The organic layer was washed with water and saturated brine, dried over anhydrous magnesium sulfate and concentrated under reduced pressure. The residue was separated and purified by column chromatography (eluent, hexane:ethyl acetate=4:1→1:1) to give the title compound (303 mg) as pale-yellow amorphous.

¹H-NMR (CDCl₃) δ: 1.41 (9H, s), 1.46 (9H, s), 1.92-2.04 (2H, m), 2.23 (2H, t, J=7.2 Hz), 3.56 (2H, t, J=7.5 Hz), 3.81 (3H, s), 4.80 (2H, s), 5.91 (1H, br s), 6.89 (2H, d, J=8.7 Hz), 7.01 (1H, d, J=8.7 Hz), 7.02-7.06 (1H, m), 7.17 (2H, d, J=8.7 Hz), 7.30-7.44 (3H, m), 7.49 (1H, dd, J=8.7, 2.7 Hz), 7.98 (1H, d, J=2.7 Hz), 8.31 (1H, s), 9.80 (1H, s), 11.17 (1H, s).

### (vi) Production of methyl 4-[(4-{3-[(tert-butylamino)carbonyl]phenoxy}-3-chlorophenyl)amino]-9-(4-methoxybenzyl)-8,9-dihydro-7H-pyrimido[4,5-b]azepine-6-carboxylate

Using tert-butyl 4-[{6-[(4-{3-[(tert-butylamino)carbonyl]phenoxy}-3-chlorophenyl)amino]-5-formylpyrimidin-4-yl}(4-methoxybenzyl)amino]butanoate (303 mg), 28% sodium methoxide-methanol solution (0.25 g) and dimethyl carbonate (6.0 mL), a similar reaction as in Example 132 (ii) was carried out to give the title compound (235 mg) as pale-yellow amorphous.

¹H-NMR (CDCl₃) δ: 1.46 (9H, s), 2.62-2.68 (2H, m), 3.31-3.39 (2H, m), 3.79 (3H, s), 3.81 (3H, s), 4.87 (2H, s), 5.93 (1H, br s), 6.86-6.89 (3H, m), 7.02 (1H, d, J=9.0 Hz), 7.04-7.08 (1H, m), 7.23 (2H, d, J=8.4 Hz), 7.32-7.44 (4H, m), 7.69 (1H, s), 7.73 (1H, d, J=2.4 Hz), 8.23 (1H, s).

### (vii) Production of methyl 4-[(4-{3-[(tert-butylamino)carbonyl]phenoxy}-3-chlorophenyl)amino]-8,9-dihydro-7H-pyrimido[4,5-b]azepine-6-carboxylate

Using methyl 4-[(4-{3-[(tert-butylamino)carbonyl]phenoxy}-3-chlorophenyl)amino]-9-(4-methoxybenzyl)-8,9-dihydro-7H-pyrimido[4,5-b]azepine-6-carboxylate (230 mg), trifluoroacetic acid (3.0 mL) and 1,2-dichloroethane (3.0 mL), a similar reaction as in Example 132 (iii) was carried out to give the title compound (111 mg) as colorless crystals.

1H-NMR (CDCl₃) δ: 1.46 (9H, s), 2.89-2.92 (2H, m), 3.51-3.56 (2H, m), 3.83 (3H, s), 5.78-5.85 (1H, m), 5.93 (1H, br s), 6.91 (1H, s), 7.01 (1H, d, J=8.4 Hz), 7.03-7.08 (1H, m), 7.31-7.43 (4H, m), 7.68 (1H, s), 7.72 (1H, d, J=2.7 Hz), 8.11 (1H, s).

### Example 136

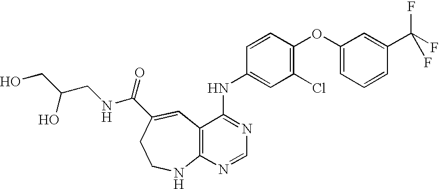

### Production of 4-({3-chloro-4-[3-(trifluoromethyl)phenoxy]phenyl}amino)-N-(2,3-dihydroxypropyl)-8,9-dihydro-7H-pyrimido[4,5-b]azepine-6-carboxamide

The mixture of 4-({3-chloro-4-[3-(trifluoromethyl)phenoxy]phenyl}amino)-8,9-dihydro-7H-pyrimido[4,5-b]azepine-6-carboxylic acid (100 mg), 3-aminopropane-1,2-diol (38 mg), 1-ethyl-3-(3-dimethylaminopropyl)carbodiimide hydrochloride (80 mg), 1-hydroxybenzotriazole monohydrate (64 mg) and triethylamine (30 μL) in N,N-dimethyl formamide (5.0 mL) was stirred at room temperature for 3 days. Water was added to reaction mixture, and the mixture was extracted with ethyl acetate. The organic layer was washed with water and saturated brine, dried over anhydrous magnesium sulfate and concentrated under reduced pressure. The residue was separated and purified by column chromatography (eluent, ethyl acetate→ethyl acetate:methanol=3:1) to give the title compound (78 mg) as colorless crystals.

¹H-NMR (DMSO-d₆) δ: 2.65-2.75 (2H, m), 3.03-3.15 (1H, m), 3.24-3.45 (5H, m), 3.52-3.63 (1H, m), 4.57 (1H, t, J=5.9 Hz), 4.82 (1H, d, J=5.1 Hz), 7.15-7.23 (3H, m), 7.27 (1H, d, J=9.0 Hz), 7.46 (1H, d, J=8.4 Hz), 7.53-7.73 (3H, m), 7.91 (1H, d, J=2.4 Hz), 7.96-8.02 (2H, m), 9.14 (1H, s).

### Example 137

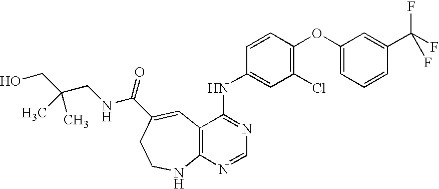

### Production of 4-({3-chloro-4-[3-(trifluoromethyl)phenoxy]phenyl}amino)-N-(3-hydroxy-2,2-dimethylpropyl)-8,9-dihydro-7H-pyrimido[4,5-b]azepine-6-carboxamide

Using 4-({3-chloro-4-[3-(trifluoromethyl)phenoxy]phenyl}amino)-8,9-dihydro-7H-pyrimido[4,5-b]azepine-6-carboxylic acid (100 mg), 3-amino-2,2-dimethylpropan-1-ol (43 mg), 1-ethyl-3-(3-dimethylaminopropyl)carbodiimide hydrochloride (80 mg), 1-hydroxybenzotriazole monohydrate (64 mg), triethylamine (30 μL) and N,N-dimethylformamide (5.0 mL), a similar reaction as in Example 136 was carried out to give the title compound (72 mg) as colorless crystals.

¹H-NMR (CDCl₃) δ: 0.93 (6H, s), 2.81 (2H, t, J=4.5 Hz), 3.23-3.27 (4H, m), 3.55-3.60 (2H, m), 3.70 (1H, t, J=6.6 Hz), 5.67-5.75 (1H, m), 6.23-6.31 (1H, m), 7.05 (1H, d, J=8.7 Hz), 7.06-7.14 (2H, m), 7.22 (1H, br s), 7.30-7.46 (3H, m), 7.50 (1H, s), 7.77 (1H, d, J=3.0 Hz), 8.14 (1H, s).

### Example 138

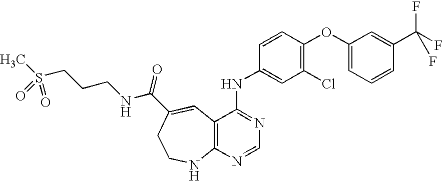

### Production of 4-({3-chloro-4-[3-(trifluoromethyl)phenoxy]phenyl}amino)-N-[3-(methylsulfonyl)propyl]-8,9-dihydro-7H-pyrimido[4,5-b]azepine-6-carboxamide

### (i) Production of tert-butyl [3-(methylsulfonyl)propyl]carbamate

A solution of tert-butyl (3-bromopropyl)carbamate (2.05 g), sodium methanesulfinate (1.05 g) and pyridine (0.83 g) in N,N-dimethylformamide (20 mL) was stirred at 60° C. for 2 days. Water was added to the reaction mixture, and the mixture was extracted with ethyl acetate. The organic layer was washed with water and saturated brine, dried over anhydrous magnesium sulfate and concentrated under reduced pressure. The residue was separated and purified by column chromatography (eluent, hexane:ethyl acetate=1:1→1:4) to give the title compound (1.34 g) as a white solid.

¹H-NMR (CDCl₃) δ: 1.44 (9H, s), 2.01-2.11 (2H, m), 2.93 (3H, s), 3.05-3.10 (2H, m), 3.27-3.33 (2H, m), 4.62-4.80 (1H, m).

### (ii) Production of 3-(methylsulfonyl)-1-propylamine hydrochloride

To a solution of tert-butyl [3-(methylsulfonyl)propyl]carbamate (1.34 g) in tetrahydrofuran (20 mL) was added 6N hydrochloric acid (5.0 mL) at room temperature. The mixture was stirred at 60° C. for 20 hr, and concentrated under reduced pressure. Ethanol was added to the residue and the mixture was concentrated again. The precipitated crystals were collected by filtration. The crystals were washed with diisopropyl ether to give the title compound (0.89 g) as colorless crystals.

¹H-NMR (DMSO-d₆) δ: 1.95-2.05 (2H, m), 2.91 (2H, t, J=7.5 Hz), 3.00 (3H, s), 3.26 (2H, t, J=7.8 Hz), 8.04 (3H, br s).

### (iii) Production of 4-({3-chloro-4-[3-(trifluoromethyl)phenoxy]phenyl}amino)-N-[3-(methylsulfonyl)propyl]-8,9-dihydro-7H-pyrimido[4,5-b]azepine-6-carboxamide

Using 4-({3-chloro-4-[3-(trifluoromethyl)phenoxy]phenyl}amino)-8,9-dihydro-7H-pyrimido[4,5-b]azepine-6-carboxylic acid (100 mg), 3-(methylsulfonyl)-1-propylamine hydrochloride (73 mg), 1-ethyl-3-(3-dimethylaminopropyl)carbodiimide hydrochloride (80 mg), 1-hydroxybenzotriazole monohydrate (64 mg), triethylamine (30 μL) and N,N-dimethylformamide (5.0 mL), a similar reaction as in Example 136 was carried out to give the title compound (91 mg) as colorless crystals.

¹H-NMR (CDCl₃) δ: 2.16-2.26 (2H, m), 2.87 (2H, t, J=4.8 Hz), 2.95 (3H, s), 3.23 (2H, t, J=6.3 Hz), 3.52-3.60 (4H, m), 5.69-5.76 (1H, m), 6.76-6.84 (1H, m), 7.06 (1H, d, J=8.7 Hz), 7.08-7.12 (1H, m), 7.15-7.20 (1H, m), 7.22-7.28 (1H, m), 7.30-7.34 (1H, m), 7.40-7.45 (2H, m), 7.53 (1H, dd, J=8.7, 2.4 Hz), 7.75 (1H, d, J=2.4 Hz), 8.12 (1H, s).

### Example 139

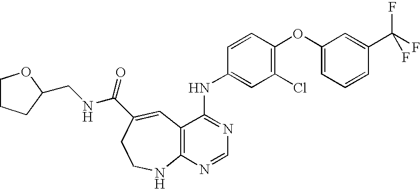

### Production of 4-({3-chloro-4-[3-(trifluoromethyl)phenoxy]phenyl}amino)-N-(tetrahydrofuran-2-ylmethyl)-8,9-dihydro-7H-pyrimido[4,5-b]azepine-6-carboxamide

Using 4-({3-chloro-4-[3-(trifluoromethyl)phenoxy]phenyl}amino)-8,9-dihydro-7H-pyrimido[4,5-b]azepine-6-carboxylic acid (100 mg), tetrahydrofurfurylamine (43 mg), 1-ethyl-3-(3-dimethylaminopropyl)carbodiimide hydrochloride (80 mg), 1-hydroxybenzotriazole monohydrate (64 mg), triethylamine (88 μL) and N,N-dimethylformamide (5.0 mL), a similar reaction as in Example 136 was carried out to give the title compound (102 mg) as colorless crystals.

¹H-NMR (CDCl₃) δ: 1.50-1.70 (2H, m), 1.84-2.08 (2H, m), 2.82 (2H, t, J=4.8 Hz), 3.17-3.26 (1H, m), 3.53-3.58 (2H, m), 3.70-3.92 (3H, m), 3.99-4.08 (1H, m), 5.66-5.73 (1H, m), 6.25-6.33 (1H, m), 7.04 (1H, d, J=8.4 Hz), 7.05-7.11 (1H, m), 7.21 (1H, br s), 7.30-7.33 (1H, m), 7.39-7.48 (4H, m), 7.78 (1H, d, J=2.7 Hz), 8.12 (1H, br s).

### Example 140

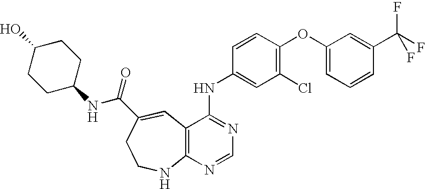

### Production of 4-({3-chloro-4-[3-(trifluoromethyl)phenoxy]phenyl}amino)-N-(trans-4-hydroxycyclohexyl)-8,9-dihydro-7H-pyrimido[4,5-b]azepine-6-carboxamide

Using 4-({3-chloro-4-[3-(trifluoromethyl)phenoxy]phenyl}amino)-8,9-dihydro-7H-pyrimido[4,5-b]azepine-6-carboxylic acid (100 mg), trans-4-aminocyclohexanol (48 mg), 1-ethyl-3-(3-dimethylaminopropyl) carbodiimide hydrochloride (80 mg), 1-hydroxybenzotriazole monohydrate (64 mg), triethylamine (88 μl) and N,N-dimethylformamide (5.0 mL), a Similar reaction as in Example 136 was carried out to give, the title compound (97 mg) as colorless crystals.

¹H-NMR (CDCl₃) δ: 1.18-1.60 (4H, m), 1.98-2.11 (4H, m), 2.76 (2H, t, J=4.5 Hz), 3.51-3.69 (2H, m), 3.76-3.93 (2H, m), 5.45-5.51 (1H, m), 5.66-5.74 (1H, m), 7.04 (1H, d, J=9.0 Hz), 7.06-7.11 (1H, m), 7.18-7.23 (1H, m), 7.30-7.35 (1H, m), 7.38-7.44 (4H, m), 7.78 (1H, d, J=2.7 Hz), 8.12 (1H, s).

### Example 141

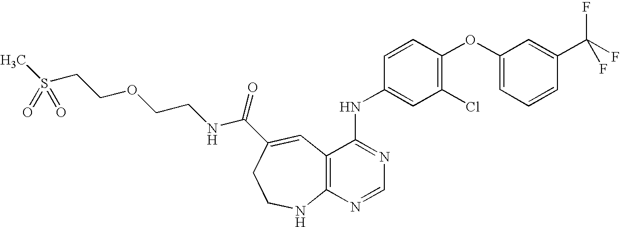

### Production of 4-({3-chloro-4-[3-(trifluoromethyl)phenoxy]phenyl}amino)-N-{2-[2-(methylsulfonyl)ethoxy]ethyl}-8,9-dihydro-7H-pyrimido[4,5-b]azepine-6-carboxamide

### (i) Production of tert-butyl [2-(2-iodoethoxy)ethyl]carbamate

To a solution of tert-butyl [2-(2-hydroxyethoxy)ethyl]carbamate (2.1 g),

triphenylphosphine (3.21 g) and imidazole (0.83 g) in dichloromethane (40 mL) was added iodine (2.85 g) at 0° C. The mixture was stirred at room temperature for 20 hr. Water was added to the mixture and the mixture was extracted with ethyl acetate. The organic layer was washed with saturated brine, dried over anhydrous magnesium sulfate and concentrated under reduced pressure. Diethyl ether was added to the residue, and the precipitated crystals were removed by filtration.

The filtrate was concentrated, and the residue was separated and purified by column chromatography (eluent, hexane:ethyl acetate=9:1->3:1) to give the title compound (2.55 g) as a colorless oil.

¹H-NMR (CDCl₃) δ: 1.45 (9H, s), 3.26 (2H, t, J=6.6 Hz), 3.30-3.35 (2H, m), 3.55 (2H, t, J=5.3 Hz), 3.71 (2H, t, J=6.6 Hz), 4.83-5.01 (1H, m).

### (ii) Production of tert-butyl {2-[2-(methylsulfonyl)ethoxy]ethyl}carbamate

Using tert-butyl [2-(2-iodoethoxy)ethyl]carbamate (2.55 g), sodium methanesulfinate (1.07 g), pyridine (0.85 mL) and N,N-dimethylformamide (30 mL), a similar reaction as in Example 138 (i) was carried out to give the title compound (2.05 g) as a colorless oil.

¹H-NMR (CDCl₃) δ: 1.44 (9H, s), 3.00 (3H, s), 3.23 (2H, t, J=5.3 Hz), 3.28-3.38 (2H, m), 3.56 (2H, t, J=5.4 Hz), 3.91 (2H, t, J=5.4 Hz), 4.74-4.87 (1H, m).

### (iii) Production of 2-[2-(methylsulfonyl)ethoxy]ethylamine hydrochloride

Using tert-butyl {2-[2-(methylsulfonyl)ethoxy]ethyl}carbamate (2.05 g), 6N hydrochloric acid (5.0 mL) and tetrahydrofuran (20 mL), a similar reaction as in Example 138 (ii) was carried out to give the title compound (1.43 g) as colorless crystals.

¹H-NMR (DMSO-d₆) δ: 2.98 (2H, t, J=5.4 Hz), 3.02 (3H, s), 3.40 (2H, t, J=5.7 Hz), 3.64 (2H, t, J=5.4 Hz), 3.82 (2H, t, J=5.7 Hz), 8.00 (3H, br s).

### (iv) Production of 4-({3-chloro-4-[3-(trifluoromethyl)phenoxy]phenyl}amino)-N-{2-[2-(methylsulfonyl)ethoxy]ethyl}-8,9-dihydro-7H-pyrimido[4,5-b]azepine-6-carboxamide

Using 4-({3-chloro-4-[3-(trifluoromethyl)phenoxy]phenyl}amino)-8,9-dihydro-7H-pyrimido[4,5-b]azepine-6-carboxylic acid (100 mg), 2-[2-(methylsulfonyl)ethoxy]ethylamine hydrochloride (85.5 mg), 1-ethyl-3-(3-dimethylaminopropyl)carbodiimide hydrochloride (80.6 mg), 1-hydroxybenzotriazole monohydrate (64 mg), triethylamine (88 μL) and N,N-dimethyl formamide (5.0 mL), a similar reaction as in Example 136 was carried out to give the title compound (97 mg) as colorless crystals.

¹H-NMR (CDCl₃) δ: 2.86-2.89 (2H, m), 2.94 (3H, s), 3.32 (2H, t, J=5.1 Hz), 3.54-3.64 (4H, m), 3.70 (2H, t, J=4.5 Hz), 4.00 (2H, t, J=5.1 Hz), 5.67-5.75 (1H, m), 6.80-6.88 (1H, m), 7.05 (1H, d, J=8.7 Hz), 7.08-7.11 (1H, m), 7.15-7.22 (2H, m), 7.27-7.31 (1H, m), 7.39-7.45 (1H, m), 7.47 (1H, dd, J=8.7, 2.7 Hz), 7.55 (1H, s), 7.74 (1H, d, J=2.7 Hz), 8.10 (1H, s).

### Example 142

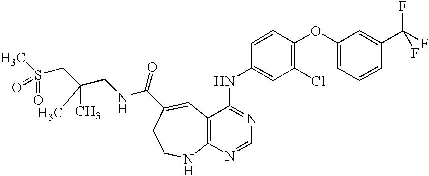

### Production of 4-({3-chloro-4-[3-(trifluoromethyl)phenoxy]phenyl}amino)-N-[2,2-dimethyl-3-(methylsulfonyl)propyl]-8,9-dihydro-7H-pyrimido[4,5-b]azepine-6-carboxamide

### (i) Production of tert-butyl [2,2-dimethyl-3-(methylthio)propyl]carbamate

To a solution of tert-butyl (3-hydroxy-2,2-dimethylpropyl)carbamate (3.0 g) and triethylamine (4.1 mL) in tetrahydrofuran (50 mL) was added methanesulfonyl chloride (1.71 mL) at 0° C. The mixture was stirred at 0° C. for 4 hr. Aqueous sodium bicarbonate solution was added to the reaction mixture and the mixture was extracted with ethyl acetate. The organic layer was washed with water and saturated brine, dried over anhydrous magnesium sulfate and concentrated under reduced pressure. To a solution of the residue in N,N-dimethylformamide (50 mL) was added sodium methanethiolate (1.03 g) at room temperature. The mixture was stirred at 70° C. for 18 hr. Sodium methanethiolate (0.31 g) was added to the reaction mixture and the mixture was further stirred at 70° C. for 20 hr. Water was added to reaction mixture, and the mixture was extracted with ethyl acetate. The organic layer was washed with water and saturated brine, dried over anhydrous magnesium sulfate and concentrated under reduced pressure. The residue was separated and purified by column chromatography (eluent, hexane:ethyl acetate=9:1→2:1) to give the title compound (2.99 g) as a colorless oil.

¹H-NMR (CDCl₃) δ: 0.97 (6H, s), 1.47 (9H, s), 2.12 (3H, s), 2.45 (2H, s), 3.05 (2H, d, J=6.6 Hz), 4.61-4.75 (1H, m).

### (ii) Production of tert-butyl [2,2-dimethyl-3-(methylsulfonyl)propyl]carbamate

To a solution of tert-butyl [2,2-dimethyl-3-(methylthio)propyl]carbamate (2.99 g) in dichloromethane (120 mL) was added 3-chloroperbenzoic acid (70%, 6.63 g) at 0° C. The mixture was stirred at 0° C. for 2 hr. Aqueous sodium thiosulfate solution was added to the reaction mixture, and the mixture was stirred at room temperature for 30 min. The mixture was extracted with ethyl acetate, and the organic layer was washed with aqueous sodium bicarbonate solution, saturated brine, dried over anhydrous magnesium sulfate and concentrated under reduced pressure. The residue was separated and purified by column chromatography (eluent, hexane:ethyl acetate=3:1→4:1→ethyl acetate) to give the title compound (3.46 g) as colorless, crystals.

¹H-NMR (CDCl₃) δ: 1.19 (6H, s), 1.44 (9H, s), 2.94 (3H, s), 3.00 (2H, s), 3.23 (2H, d, J=7.2 Hz), 5.03-5.15 (1H, m).

### (iii) Production of 2,2-dimethyl-3-(methylsulfonyl)-1-propylamine hydrochloride

Using tert-butyl [2,2-dimethyl-3-(methylsulfonyl)propyl]carbamate (3.46 g), 6N hydrochloric acid (20 mL) and tetrahydrofuran (60 mL), a similar reaction as in Example 138 (ii) was carried out to give the title compound (2.44 g) as colorless crystals.

¹H-NMR (DMSO-d₆) δ: 1.17 (6H, s), 2.89-2.98 (2H, m), 3.01 (3H, s), 3.42 (2H, s), 8.16 (3H, br s).

### (iv) Production of 4-({3-chloro-4-[3-(trifluoromethyl)phenoxy]phenyl}amino)-N-[2,2-dimethyl-3-(methylsulfonyl)propyl]-8,9-dihydro-7H-pyrimido[4,5-b]azepine-6-carboxamide

Using 4-({3-chloro-4-[3-(trifluoromethyl)phenoxy]phenyl}amino)-8,9-dihydro-7H-pyrimido[4,5-b]azepine-6-carboxylic acid (100 mg), 2,2-dimethyl-3-(methylsulfonyl)-1-propylamine hydrochloride (85 mg), 1-ethyl-3-(3-dimethylaminopropyl)carbodiimide hydrochloride (80.6 mg), 1-hydroxybenzotriazole monohydrate (64 mg), triethylamine (88 μl) and N,N-dimethylformamide (5.0 mL), a similar reaction as in Example 136 was carried out to give the title compound (92 mg) as colorless crystals.

¹H-NMR (CDCl₃) δ: 1.24 (6H, s), 2.85-2.95 (5H, m), 3.10 (2H, s), 3.47-3.58 (4H, m), 5.68-5.75 (1H, m), 7.05 (1H, d, J=8.7 Hz), 7.08-7.18 (2H, m), 7.26 (1H, s), 7.29-7.45 (4H, m), 7.56 (1H, dd, J=8.7, 2.4 Hz), 7.71 (1H, d, J=2.4 Hz), 8.10 (1H, s).

### Example 143

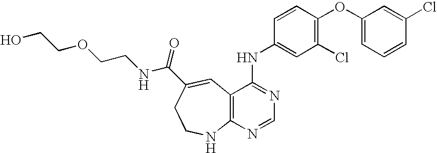

### Production of 4-{[3-chloro-4-(3-chlorophenoxy)phenyl]amino}-N-[2-(2-hydroxyethoxy)ethyl]-8,9-dihydro-7H-pyrimido[4,5-b]azepine-6-carboxamide hydrochloride

Using 4-{[3-chloro-4-(3-chlorophenoxy)phenyl]amino}-8,9-dihydro-7H-pyrimido[4,5-b]azepine-6-carboxylic acid (106 mg), 2-(2-aminoethoxy)ethanol (57 mg), 1-ethyl-3-(3-dimethylaminopropyl)carbodiimide hydrochloride (104 mg), 1-hydroxybenzotriazole monohydrate (83 mg), triethylamine (0.11 mL) and N,N-dimethylformamide (5.0 mL), a similar reaction as in Example 136 was carried out to give 4-{[3-chloro-4-(3-chlorophenoxy)phenyl]amino}-N-[2-(2-hydroxyethoxy)ethyl]-8,9-dihydro-7H-pyrimido[4,5-b]azepine-6-carboxamide. To a solution of 4-{[3-chloro-4-(3-chlorophenoxy)phenyl]amino}-N-[2-(2-hydroxyethoxy)ethyl]-8,9-dihydro-7H-pyrimido[4,5-b]azepine-6-carboxamide in ethanol (2.0 mL) was added 4N hydrochloric acid/ethyl acetate (0.5 mL) at room temperature. The mixture was concentrated under reduced pressure and crystallized from ethanol-ethyl acetate to give the title compound (80 mg) as pale-yellow crystals.

¹H-NMR (DMSO-d₆) δ: 2.70-2.79 (2H, m), 3.27-3.37 (2H, m), 3.40-3.54 (8H, m), 6.91-6.94 (1H, m), 7.00-7.02 (1H, m), 7.18-7.22 (2H, m), 7.29 (1H, d, J=9.0 Hz), 7.39-7.45 (1H, m), 7.52 (1H, dd, J=9.0, 2.5 Hz), 7.80 (1H, d, J= 2.5 Hz), 8.21 (1H, s), 8.41-8.50 (1H, m), 8.52-8.69 (1H, m), 10.18-10.31 (1H, m).

### Example 144

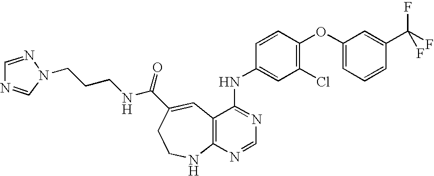

### Production of 4-({3-chloro-4-[3-(trifluoromethyl)phenoxy]phenyl}amino)-N-[3-(1H-1,2,4-triazol-1-yl)propyl]-8,9-dihydro-7H-pyrimido[4,5-b]azepine-6-carboxamide

### (i) Production of tert-butyl [3-(1H-1,2,4-triazol-1-yl)propyl]carbamate

A mixture of tert-butyl (3-bromopropyl)carbamate (1.02 g), 1H-1,2,4-triazole (0.35 g), potassium carbonate (0.87 g) in acetone (30 mL) was stirred at 70° C. for 12 hr, and concentrated under reduced pressure. Water was added to the residue and the mixture was extracted with ethyl acetate. The organic layer was washed with saturated brine, dried over anhydrous magnesium sulfate and concentrated under reduced pressure. The residue was separated and purified by column chromatography (eluent, ethyl acetate->ethyl acetate:methanol=97:3→4:1) to give the title compound (0.93 g) as a colorless oil.

¹H-NMR (CDCl₃) δ: 1.45 (9H, s), 2.02-2.11 (2H, m), 3.16 (2H, td, J=6.3, 6.3 Hz), 4.24 (2H, t, J=6.8 Hz), 4.63-4.81 (1H, m), 7.95 (1H, s), 8.14 (1H, s).

### (ii) Production of 3-(1H-1,2,4-triazol-1-yl)-1-propylamine hydrochloride

Using tert-butyl [3-(1H-1,2,4-triazol-1-yl)propyl]carbamate (0.93 g), 6N hydrochloric acid (5.0 mL) and tetrahydrofuran (10 mL), a similar reaction as in Example 138 (ii) was carried out to give the title compound (761 mg) as colorless crystals.

¹H-NMR (DMSO-d₆) δ: 2.05-2.14 (2H, m), 2.70-2.83 (2H, m), 4.33 (2H, t, J=6.8 Hz), 8.10 (3H, br s), 8.14 (1H, s), 8.76 (1H, s).

### (iii) Production of 4-({3-chloro-4-[3-(trifluoromethyl)phenoxy]phenyl}amino)-N-[3-(1H-1,2,4-triazol-1-yl)propyl]-8,9-dihydro-7H-pyrimido[4,5-b]azepine-6-carboxamide

Using 4-({3-chloro-4-[3-(trifluoromethyl)phenoxy]phenyl}amino)-8,9-dihydro-7H-pyrimido[4,5-b]azepine-6-carboxylic acid (100 mg), 3-(1H-1,2,4-triazol-1-yl)-1-propylamine hydrochloride (84 mg), 1-ethyl-3-(3-dimethylaminopropyl)carbodiimide hydrochloride (80.6 mg), 1-hydroxybenzotriazole monohydrate (64 mg), triethylamine (88 μL) and N,N-dimethylformamide (5.0 mL), a similar reaction as in Example 136 was carried out to give the title compound (58 mg) as colorless crystals.

¹H-NMR (CDCl₃) δ: 2.11-2.23 (2H, m), 2.76-2.83 (2H, m), 3.43-4.50 (2H, m), 3.52-3.57 (2H, m), 4.32 (2H, t, J=6.3 Hz), 5.66-5.72 (1H, m), 6.10-6.20 (1H, m), 7.04 (1H, d, J=8.7 Hz), 7.06-7.12 (1H, m), 7.19-7.22 (1H, m), 7.23-7.35 (2H, m), 7.40-7.46 (2H, m), 7.52 (1H, br s), 7.80 (1H, d, J=2.7 Hz), 7.93 (1H, s), 8.13 (1H, s), 8.17 (1H, s).

### Example 145

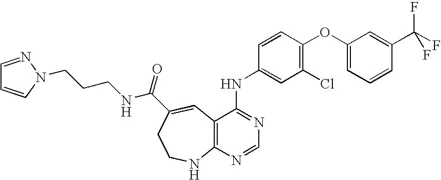

### Production of 4-({3-chloro-4-[3-(trifluoromethyl)phenoxy]phenyl}amino)-N-[3-(1H-pyrazol-1-yl)propyl]-8,9-dihydro-7H-pyrimido[4,5-b]azepine-6-carboxamide

### (i) Production of tert-butyl [3-(1H-pyrazol-1-yl)propyl]carbamate

To a solution of pyrazole (0.35 g) in tetrahydrofuran (15 mL) was added 60% sodium hydride (dispersion in mineral oil, 0.22 g) at 0° C. The mixture was stirred at 0° C. for 1 hr, and tert-butyl (3-bromopropyl)carbamate (1.05 g) was added thereto. The mixture was stirred at 50° C. for 20 hr. Water was added to the mixture and the mixture was extracted with ethyl acetate. The organic layer was washed with water and saturated brine, dried over anhydrous magnesium sulfate and concentrated under reduced pressure. The residue was separated and purified by column chromatography (eluent, hexane:ethyl acetate=1:1→ethyl acetate) to give the title compound (0.96 g) as a colorless oil.

¹H-NMR (CDCl₃) δ: 1.44 (9H, s), 1.98-2.07 (2H, m), 3.07-3.13 (2H, m), 4.19 (2H, t, J=6.6 Hz), 4.68-4.82 (1H, m), 6.24 (1H, dd, J=2.1, 1.5 Hz), 7.40 (1H, d, J=2.1 Hz), 7.49 (1H, d, J=1.5 Hz).

### (ii) Production of 3-(1H-pyrazol-1-yl)-1-propylamine hydrochloride

Using tert-butyl [3-(1H-pyrazol-1-yl)propyl]carbamate (0.96 g), 6N hydrochloric acid (5.0 mL) and tetrahydrofuran (10 mL), a similar reaction as in Example 138 (ii) was carried out to give the title compound (747 mg) as colorless crystals.

¹H-NMR (DMSO-d₆) δ: 2.01-2.11 (2H, m), 2.64-2.79 (2H, m), 4.23 (2H, t, J=6.8 Hz), 6.25 (1H, dd, J=2.1, 1.8 Hz), 7.47 (1H, d, J=1.8 Hz), 7.78 (1H, d, J=2.1 Hz), 8.11 (3H, br s).

(iii) Production of 4-({3-chloro-4-[3-(trifluoromethyl)phenoxy]phenyl}amino)-N-[3-(1H-pyrazol-1-yl)propyl]-8,9-dihydro-7H-pyrimido[4,5-b]azepine-6-carboxamide

Using 4-({3-chloro-4-[3-(trifluoromethyl)phenoxy]phenyl}amino)-8,9-dihydro-7H-pyrimido[4,5-b]azepine-6-carboxylic acid (100 mg), 3-(1H-pyrazol-1-yl)-1-propylamine hydrochloride (83.2 mg), 1-ethyl-3-(3-dimethylaminopropyl)carbodiimide hydrochloride (80.6 mg), 1-hydroxybenzotriazole monohydrate (64 mg), triethylamine (88 μL) and N,N-dimethylformamide (5.0 mL), a similar reaction as in Example 136 was carried out to give the title compound (100 mg) as colorless crystals.

¹H-NMR (CDCl₃) δ: 2.06-2.17 (2H, m), 2.82 (2H, t, J=204.5 Hz), 3.33-3.84 (2H, m), 3.53-3.57 (2H, m), 4.30 (2H, t, J=6.2 Hz), 5.66-5.75 (1H, m), 6.28-6.29 (1H, m), 6.95-7.06 (2H, m), 7.08-7.12 (1H, m), 7.18-7.22 (1H, m), 7.29-7.35 (1H, m), 7.36-7.48 (6H, m), 7.74 (1H, d, J=2.4 Hz), 8.13 (1H, s).

### Example 146

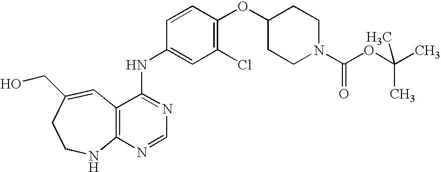

### Production of tert-butyl 4-(2-chloro-4-{[6-(hydroxymethyl)-8,9-dihydro-7H-pyrimido[4,5-b]azepin-4-yl]amino}phenoxy)piperidine-1-carboxylate

### (i) Production of 4-[(4-{[1-(tert-butoxycarbonyl)piperidin-4-yl]oxy}-3-chlorophenyl)amino]-8,9-dihydro-7H-pyrimido[4,5-b]azepine-6-carboxylic acid

To a solution of methyl 4-[(4-{[1-(tert-butoxycarbonyl)piperidin-4-yl]oxy}-3-chlorophenyl)amino]-8,9-dihydro-7H-pyrimido[4,5-b]azepine-6-carboxylate (1.5 g) in tetrahydrofuran (30 mL) and ethanol (30 mL) was added 1N aqueous sodium hydroxide solution (10 mL) at room temperature. The mixture was stirred at room temperature for 3 days. 1N hydrochloric acid (10 mL) was added to the reaction mixture, and the mixture was concentrated under reduced pressure. The precipitated crystals were collected by filtration and washed with water to give the title compound (1.34 g) as pale-yellow crystals.

¹H-NMR (DMSO-d₆) δ: 1.41 (9H, s), 1.51-1.65 (2H, m), 1.81-1.91 (2H, m), 2.64-2.75 (2H, m), 3.21-3.41 (4H, m), 3.53-3.64 (2H, m), 4.50-4.59 (1H, m), 7.15 (1H, d, J=9.0 Hz), 7.37 (1H, dd, J=9.0, 2.7 Hz), 7.61 (1H, d, J=2.7 Hz), 7.67 (1H, s), 7.77 (1H, t, J=5.0 Hz), 7.91 (1H, s), 9.13 (1H, s).

### (ii) Production of tert-butyl 4-(2-chloro-4-{[6-(hydroxymethyl)-8,9-dihydro-7H-pyrimido[4,5-b]azepin-4-yl]amino}phenoxy)piperidine-1-carboxylate

To a solution of 4-[(4-{[1-(tert-butoxycarbonyl)piperidin-4-yl]oxy}-3-chlorophenyl)amino]-8,9-dihydro-7H-pyrimido[4,5-b]azepine-6-carboxylic acid (0.50 g) and 4-methylmorpholine (0.122 mL) in tetrahydrofuran (5.0 mL) was added isopropyl chlorocarbonate (0.138 mL) at 0° C. The mixture was stirred at 0° C. for 2 hr, and an insoluble material was removed by filtration. The filtrate was added dropwise to a mixed solution of sodium borohydride (37 mg) in tetrahydrofuran (3.0 mL) and water (3.0 mL) at 0° C. The mixture was stirred at 0° C. for 1 hr and extracted with ethyl acetate. The organic layer was washed with water and saturated brine, dried over anhydrous magnesium sulfate and concentrated under reduced pressure. The residue was separated and purified by column chromatography (eluent, ethyl acetate→ethyl acetate:methanol=85:15) to give the title compound (368 mg) as pale-yellow crystals.

¹H-NMR (CDCl₃) δ: 1.47 (9H, s), 1.50-1.66 (1H, m), 1.75-1.95 (3H, m), 2.53 (2H, t, J=4.8 Hz), 3.33-3.44 (2H, m), 3.49-3.54 (2H, m), 3.62-3.73 (2H, m), 4.23 (2H, s), 4.41-4.50 (1H, m), 5.45-5.52 (1H, m), 6.32 (1H, s), 6.48 (1H, s), 6.93 (1H, d, J=8.7 Hz), 7.26 (1H, dd, J=8.7, 2.7 Hz), 7.52 (1H, d, J=2.7 Hz), 8.06 (1H, s).

### Example 147

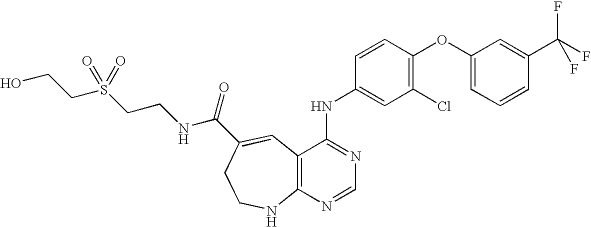

### Production of 4-({3-chloro-4-[3-(trifluoromethyl)phenoxy]phenyl}amino)-N-{2-[(2-hydroxyethyl)sulfonyl]ethyl}-8,9-dihydro-7H-pyrimido[4,5-b]azepine-6-carboxamide

### (i) Production of tert-butyl {2-[(2-hydroxyethyl)thio]ethyl}carbamate

To a solution of tert-butyl (2-bromoethyl)carbamate (2.48 g) and 2-mercaptoethanol (0.87 g) in ethanol (25 mL) was added a solution of sodium methoxide in methanol (28%, 2.15 g) at room temperature. The mixture was stirred at room temperature for 3 days and concentrated under reduced pressure. Water was added to the residue, and the mixture was extracted with ethyl acetate. The organic layer was washed with water and saturated brine, dried over anhydrous magnesium sulfate and concentrated under reduced pressure. The residue was separated and purified by column chromatography (eluent, hexane:ethyl acetate=4:1→ethyl acetate) to give the title compound (2.18 g) as a colorless oil.

¹H-NMR (CDCl₃) δ: 1.45 (9H, s), 2.2-2.38 (1H, m), 2.66

(2H, t, J=6.6 Hz), 2.75 (2H, t, J=5.7 Hz), 3.28-3.39 (2H, m), 3.72-3.78 (2H, m), 4.79-4.96 (1H, m).

(ii) Production of tert-butyl {2-[(2-hydroxyethyl)sulfonyl]ethyl}carbamate

Using tert-butyl 2-[(2-hydroxyethyl)thio]ethyl}carbamate (1.18 g), 3-chloroperbenzoic acid (70%, 2.89 g) and dichloromethane-(20 mL), a similar reaction as in Example 142 (ii) was carried out to give the title compound (1.31 g) as a colorless oil.

¹H-NMR (CDCl₃) δ: 1.44 (9H, s), 2.31-2.65 (1H, m), 3.25 (2H, t, J=5.3 Hz), 3.34 (2H, t, J=6.1 Hz), 3.62-3.69 (2H, m), 4.14 (2H, t, J=5.3 Hz), 5.09-5.16 (1H, m).

(iii) Production of 2-[(2-aminoethyl)sulfonyl]ethanol hydrochloride

Using tert-butyl {2-[(2-hydroxyethyl)sulfonyl]ethyl}carbamate (1.31 g), 6N hydrochloric acid (3.0 mL) and tetrahydrofuran (20 mL), a similar reaction as in Example 138 (ii) was carried out to give the title compound (927 mg) as colorless crystals.

¹H-NMR (DMSO-d₆) δ: 3.22 (2H, t, J=7.4 Hz), 3.37 (2H, t, J=5.6 Hz), 3.50 (2H, t, J=7.4 Hz), 3.80 (2H, t, J=5.6 Hz), 7.85-8.35 (3H, m).

### (iv) Production of 4-({3-chloro-4-[3-(trifluoromethyl)phenoxy]phenyl}amino)-N-{2-[(2-hydroxyethyl)sulfonyl]ethyl}-8,9-dihydro-7H-pyrimido[4,5-b]azepine-6-carboxamide

Using 4-({3-chloro-4-[3-(trifluoromethyl)phenoxy]phenyl}amino)-8,9-dihydro-7H-pyrimido[4,5-b]azepine-6-carboxylic acid (100 mg), 2-[(2-aminoethyl)sulfonyl]ethanol hydrochloride (80 mg), 1-ethyl-3-(3-dimethylaminopropyl)carbodiimide hydrochloride (80.6 mg), 1-hydroxybenzotriazole monohydrate (64 mg), triethylamine (88 μL) and N,N-dimethylformamide (5.0 mL), a similar reaction as in Example 136 was carried out to give the title compound (100 mg) as colorless crystals.

¹H-NMR (CDCl₃) δ: 2.52-2.64 (1H, m), 2.84 (2H, t, J=4.7 Hz), 3.29 (2H, t, J=5.1 Hz), 3.41-3.45 (2H, m), 3.50-3.55 (2H, m), 3.93-3.99 (2H, m), 4.15 (2H, t, J=5.1 Hz), 5.77 (1H, t, J=4.8 Hz), 6.74 (1H, t, J=6.3 Hz), 7.03 (1H, d, J=9.0 Hz), 7.05-7.11 (1H, m), 7.18-7.23 (1H, m), 7.30-7.45 (4H, m), 7.53 (1H, dd, J=9.0, 2.7 Hz), 7.80 (1H, d, J=2.7 Hz), 8.12 (1H, s).

### Example 148

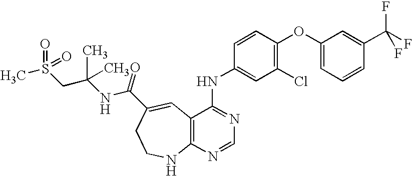

### Production of 4-({3-chloro-4-[3-(trifluoromethyl)phenoxy]phenyl}amino)-N-[1,1-dimethyl-2-(methylsulfonyl)ethyl]-8,9-dihydro-7H-pyrimido[4,5-b]azepine-6-carboxamide

### (i) Production of tert-butyl [1,1-dimethyl-2-(methylthio)ethyl]carbamate

Using tert-butyl (2-hydroxy-1,1-dimethylethyl)carbamate (2.50 g), methanesulfonyl chloride (1.53 mL), triethylamine (3.68 mL), tetrahydrofuran (75 mL), sodium methanethiolate (1.25 g) and N,N-dimethylformamide (30 mL), a similar reaction as in Example 142 (i) was carried out to give the title compound (1.94 g) as a colorless oil.

¹H-NMR (CDCl₃) δ: 1.34 (6H, s), 1.43 (9H, s), 2.15 (3H, s), 2.88 (2H, s), 4.65 (1H, br s).

### (ii) Production of tert-butyl [1,1-dimethyl-2-(methylsulfonyl)ethyl]carbamate

Using tert-butyl [1,1-dimethyl-2-(methylthio)ethyl]carbamate (1.94 g), 3-chloroperbenzoic acid (70%, 4.79 g) and dichloromethane (50 mL), a similar reaction as in Example 142 (ii) was carried out to give the title compound (2.22 g) as a colorless oil.

¹H-NMR (CDCl₃) δ: 1.44 (9H, s), 1.52 (6H, s), 2.92 (3H, s), 3.58 (2H, s), 4.79 (1H, s).

### (iii) Production of 2-methyl-1-(methylsulfonyl)-2-propylamine hydrochloride

Using tert-butyl [1,1-dimethyl-2-(methylsulfonyl)ethyl]carbamate (2.2 g), 6N hydrochloric acid (3.0 mL) and tetrahydrofuran (20 mL), a similar reaction as in Example 138 (i) was carried out to give the title compound (1.55 g) as colorless crystals.

¹H-NMR (DMSO-d₆) δ: 1.47 (6H, s), 3.13 (3H, s), 3.60 (2H, s), 8.30 (3H, br s).

### (iv) Production of 4-({3-chloro-4-[3-(trifluoromethyl)phenoxy]phenyl}amino)-N-[1,1-dimethyl-2-(methylsulfonyl)ethyl]-8,9-dihydro-7H-pyrimido[4,5-b]azepine-6-carboxamide

Using 4-({3-chloro-4-[3-(trifluoromethyl)phenoxy]phenyl}amino)-8,9-dihydro-7H-pyrimido[4,5-b]azepine-6-carboxylic acid (100 mg), 2-methyl-1-(methylsulfonyl)-2-propylamine hydrochloride (79 mg), 1-ethyl-3-(3-dimethylaminopropyl)carbodiimide hydrochloride (80.6 mg), 1-hydroxybenzotriazole monohydrate (64 mg), triethylamine (88 μL) and N,N-dimethylformamide (5.0 mL), a similar reaction as in Example 136 was carried out to give the title compound (97 mg) as colorless crystals.

¹H-NMR (CDCl₃) δ: 1.69 (6H, s), 2.84 (2H, t, J=4.4 Hz), 2.97 (3H, s), 3.50-3.55 (2H, m), 3.62 (2H, s), 5.62-5.71 (1H, m), 6.35 (1H, s), 7.03 (1H, d, J=8.7 Hz), 7.05-7.10 (1H, m), 7.16-7.33 (4H, m), 7.38-7.44 (1H, m), 7.53 (1H, dd, J=8.7, 2.7 Hz), 7.89 (1H, d, J=2.7 Hz), 8.13 (1H, s).

### Example 149

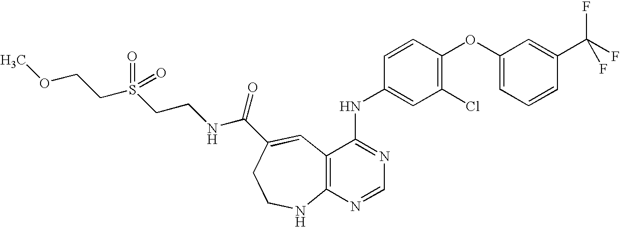

### Production of 4-({3-chloro-4-[3-(trifluoromethyl)phenoxy]phenyl}amino)-N-{2-[(2-methoxyethyl)sulfonyl]ethyl}-8,9-dihydro-7H-pyrimido[4,5-b]azepine-6-carboxamide

### (i) Production of tert-butyl {2-[(2-methoxyethyl)thio]ethyl}carbamate

To a solution of 2-aminoethanethiol (1.5 g) and 2-(bromoethyl)methyl ether (2.70 g) in ethanol (50 mL) was added a solution of sodium methoxide in methanol (28%, 3.74 g) at room temperature. The mixture was stirred at 50° C. for 18 hr, and concentrated under reduced pressure. To a solution of the residue in tetrahydrofuran (50 mL) was added di-tert-butyl dicarbonate (4.46 mL) at room temperature, and the mixture was stirred for 4 hr. 1N Hydrochloric acid (20 mL) was added to the mixture, and the mixture was extracted with ethyl acetate. The organic layer was washed with saturated brine, dried over anhydrous magnesium sulfate and concentrated under reduced pressure. The residue was separated and purified by column chromatography (eluent, hexane:ethyl acetate=9:1→1:1) to give the title compound (3.74 g) as a colorless oil.

¹H-NMR (CDCl₃) δ: 1.45 (9H, s), 2.67-2.74 (4H, m), 3.26-3.37 (2H, m), 3.37 (3H, s), 3.56 (2H, t, J=6.3 Hz), 4.93-5.08 (1H, m).

### (ii) Production of tert-butyl {2-[(2-methoxyethyl)sulfonyl]ethyl}carbamate

Using tert-butyl {2-[(2-methoxyethyl)thio]ethyl}carbamate (3.74 g), 3-chloroperbenzoic acid (70%, 7.84 g) and dichloromethane (100 mL), a similar reaction as in Example 142 (ii) was carried out to give the title compound (3.91 g) as a white solid.

¹H-NMR (CDCl₃) δ: 1.44 (9H, s), 3.22 (2H, t, J=5.3 Hz), 3.29 (2H, t, J=5.8 Hz), 3.38 (3H, s), 3.60-3.70 (2H, m), 3.81 (2H, t, J=5.3 Hz), 5.15-5.32 (1H, m).

### (iii) Production of 2-[(2-methoxyethyl)sulfonyl]ethylamine hydrochloride

Using tert-butyl {2-[(2-methoxyethyl)sulfonyl]ethyl}carbamate (3.91 g), 6N hydrochloric acid (10 mL) and tetrahydrofuran (40 mL), a similar reaction as in Example 138 (ii) was carried out to give the title compound (2.79 g) as colorless crystals.

¹H-NMR (DMSO-d₆) δ: 3.20 (2H, t, J=7.4 Hz), 3.29 (3H, s), 3.46 (2H, t, J=7.4 Hz), 3.52 (2H, t, J=3.4 Hz), 3.70 (2H, t, J=5.4 Hz), 8.12 (3H, br s).

### (iv) Production of 4-({3-chloro-4-[3-(trifluoromethyl)phenoxy]phenyl}amino)-N-{2-[(2-methoxyethyl)sulfonyl]ethyl}-8,9-dihydro-7H-pyrimido[4,5-b]azepine-6-carboxamide

Using 4-({3-chloro-4-[3-(trifluoromethyl)phenoxy]phenyl}amino)-8,9-dihydro-7H-pyrimido[4,5-b]azepine-6-carboxylic acid (100 mg), 2-[(2-methoxyethyl)sulfonyl]ethylamine hydrochloride (85.5 mg), 1-ethyl-3-(3-dimethylaminopropyl)carbodiimide hydrochloride (80.6 mg), 1-hydroxybenzotriazole monohydrate (64 mg), triethylamine (88 μL) and N,N-dimethylformamide (5.0 mL), a similar reaction as in Example 136 was carried out to give the title compound (94 mg) as colorless crystals.

¹H-NMR (CDCl₃) δ: 2.86 (2H, t, J=4.2 Hz), 3.27 (2H, t, J=5.3 Hz), 3.32-3.42 (5H, m), 3.50-3.55 (2H, m), 3.82 (2H, t, J=5.3 Hz), 3.92-3.98 (2H, m), 5.69-5.75 (1H, m), 6.66-6.75 (1H, m), 7.04 (1H, d, J=8.7 Hz), 7.05-7.10 (1H, m), 7.19-7.20 (1H, m), 7.30-7.36 (3H, m), 7.39-7.45 (1H, m), 7.56 (1H, dd, J=8.7, 2.7 Hz), 7.83 (1H, d, J=2.7 Hz), 8.13 (1H, s).

### Example 150

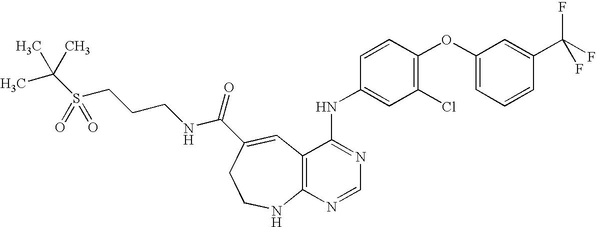

### Production of N-[3-(tert-butylsulfonyl)propyl]-4-({3-chloro-4-[3-(trifluoromethyl)phenoxy]phenyl}amino)-8,9-dihydro-7H-pyrimido[4,5-b]azepine-6-carboxamide

### (i) Production of tert-butyl [3-(tert-butyl thio)propyl]carbamate

A solution of tert-butyl (3-bromopropyl)carbamate (1.02 g) and sodium 2-methyl-2-propanethiolate (0.53 g) in ethanol (10 mL) was stirred at 60° C. for 2 days and concentrated under reduced pressure. Water was added to the residue and the mixture was extracted with ethyl acetate. The organic layer was washed with saturated brine, dried over anhydrous magnesium sulfate and concentrated under reduced pressure. The residue was separated and purified by column chromatography (eluent, hexane:ethyl acetate=9:1→2:1) to give the title compound (0.79 g) as a colorless oil.

¹H-NMR (CDCl₃) δ: 1.32 (9H, s), 1.44 (9H, s), 1.71-1.80 (2H, m), 2.55 (2H, t, J=7.2 Hz), 3.16-3.26 (2H, m), 4.53-4.68 (1H, m).

### (ii) Production of tert-butyl [3-(tert-butyl sulfonyl)propyl]carbamate

Using tert-butyl [3-(tert-butylthio)propyl]carbamate (0.79 g), 3-chloroperbenzoic acid (70%, 1.65 g) and dichloromethane (10 mL), a similar reaction as in Example 142 (ii) was carried out to give the title compound (852 mg) as a white solid.

¹H-NMR (CDCl₃) δ: 1.42 (9H, s), 1.44 (9H, s), 2.04-2.13 (2H, m), 2.97 (2H, t, J=7.5 Hz), 3.28-3.35 (2H, m), 4.67-4.80 (1H, m).

### (iii) Production of 3-(tert-butylsulfonyl)-1-propylamine hydrochloride

Using tert-butyl [3-(tert-butylsulfonyl)propyl]carbamate (852 mg), 6N hydrochloric acid (3.0 mL) and tetrahydrofuran (10 mL), a similar reaction as in Example 138 (ii) was carried out to give the title compound (611 mg) as colorless crystals.

¹H-NMR (DMSO-d₆) δ: 1.30 (9H, s), 1.91-2.04 (2H, m), 2.93 (2H, t, J=7.8 Hz), 3.21 (2H, t, J=7.8 Hz), 7.97 (3H, br s).

### (iv) Production of N-[3-(tert-butylsulfonyl)propyl]-4-({3-chloro-4-[3-(trifluoromethyl)phenoxy]phenyl}amino)-8,9-dihydro-7H-pyrimido[4,5-b]azepine-6-carboxamide

Using 4-({3-chloro-4-[3-(trifluoromethyl)phenoxy]phenyl}amino)-8,9-dihydro-7H-pyrimido[4,5-b]azepine-6-carboxylic acid (100 mg), 3-(tert-butylsulfonyl)-1-propylamine hydrochloride (90.6 mg), 1-ethyl-3-(3-dimethylaminopropyl) carbodiimide hydrochloride (80.6 mg), 1-hydroxybenzotriazole monohydrate (64 mg), triethylamine (88 μL) and N,N-dimethylformamide (5.0 mL), a similar reaction as in Example 136 was carried out to give the title compound (57 mg) as colorless crystals.

¹H-NMR (CDCl₃) δ: 1.34 (9H, s), 2.20-2.31 (2H, m), 2.88 (2H, t, J=4.7 Hz), 3.11 (2H, t, J=6.2 Hz), 3.50-3.61 (4H, m), 5.68-5.75 (1H, m), 6.92-7.01 (1H, m), 7.05 (1H, d, J=8.7 Hz), 7.10-7.17 (2H, m), 7.21-7.27 (1H, m), 7.31-7.34 (1H, m), 7.40-7.45 (2H, m), 7.52 (1H, dd, J=8.7, 2.7 Hz), 7.77 (1H, d, J=2.7 Hz), 8.11 (1H, s).

### Example 151

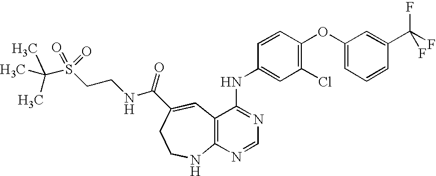

### Production of N-[2-(tert-butylsulfonyl)ethyl]-4-({3-chloro-4-[3-(trifluoromethyl)phenoxy]phenyl}amino)-8,9-dihydro-7H-pyrimido[4,5-b]azepine-6-carboxamide

### (i) Production of tert-butyl [2-(tert-butylthio)ethyl]carbamate

A solution of tert-butyl (2-bromoethyl)carbamate (2.37 g) and sodium 2-methyl-2-propanethiolate (1.32 g) in N,N-dimethylformamide (30 mL) was stirred at 40° C. for 3 days. Water was added to the reaction mixture and the mixture was extracted with hexane. The organic layer was washed with water and saturated brine, dried over anhydrous magnesium sulfate and concentrated under reduced pressure. The residue was separated and purified by column chromatography (eluent, hexane:ethyl acetate=19:1→2:1) to give the title compound (1.81 g) as a colorless oil.

¹H-NMR (CDCl₃) δ: 1.32 (9H, s), 1.44 (9H, s), 2.67 (2H, t, J=6.6 Hz), 3.24-3.35 (2H, m), 4.83-4.96 (1H, m).

### (ii) Production of tert-butyl [2-(tert-butylsulfonyl)ethyl]carbamate

Using tert-butyl [2-(tert-butylthio)ethyl]carbamate (1.81 g), 3-chloroperbenzoic acid (70%, 4.21 g) and dichloromethane (40 mL), a similar reaction as in Example 142 (ii) was carried out to give the title compound (1.94 g) as a white solid.

¹H-NMR (CDCl₃) δ: 1.42 (9H, s), 1.44 (9H, s), 3.13 (2H, t, J=5.7 Hz), 3.70-3.76 (2H, m), 5.23-5.35 (1H, m).

### (iii) Production of 2-(tert-butylsulfonyl)ethylamine hydrochloride

Using tert-butyl [2-(tert-butylsulfonyl)ethyl]carbamate (1.90 g), 2 N hydrochloric acid (10 mL) and tetrahydrofuran (20 mL), a similar reaction as in Example 138 (ii) was carried out to give the title compound (1.43 g) as colorless crystals.

¹H-NMR (DMSO-d₆) δ: 1.34 (9H, s), 3.21 (2H, t, J=7.4 Hz), 3.46 (2H, t, J=7.4 Hz), 3.09 (3H, br s).

### (iv) Production of N-[2-(tert-butylsulfonyl)ethyl]-4-({3-chloro-4-[3-(trifluoromethyl)phenoxy]phenyl}amino)-8,9-dihydro-7H-pyrimido[4,5-b]azepine-6-carboxamide

Using 4-({3-chloro-4-[3-(trifluoromethyl)phenoxy]phenyl}amino)-8,9-dihydro-7H-pyrimido[4,5-b]azepine-6-carboxylic acid (100 mg), 2-(tert-butylsulfonyl)ethylamine hydrochloride (84.7 mg), 1-ethyl-3-(3-dimethylaminopropyl)carbodiimide hydrochloride (80.6 mg), 1-hydroxybenzotriazole monohydrate (64 mg), triethylamine (88 μL) and N,N-dimethylformamide (5.0 mL), a similar reaction as in Example 136 was carried out to give the title compound (107 mg) as colorless crystals.

¹H-NMR (CDCl₃) δ: 1.42 (9H, s), 2.87 (2H, t, J=4.8 Hz), 3.17-3.21 (2H, m), 3.51 (2H, q, J=4.6 Hz), 3.99-4.05 (2H, m), 5.76 (1H, t, J=4.8 Hz), 6.72-6.80 (1H, m), 7.03 (1H, d, J=9.0 Hz), 7.06-7.11 (1H, m), 7.17 (1H, s), 7.31-7.33 (2H, m), 7.39-7.45 (2H, m), 7.60 (1H, dd, J=9.0, 2.7 Hz), 7.79 (1H, d, J=2.7 Hz), 8.12 (1H, s).

### Example 152

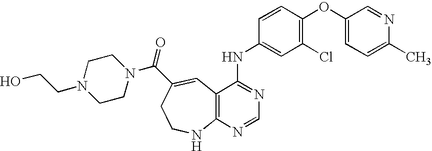

### Production of 2-(4-{[4-({3-methyl-4-[(6-methylpyridin-3-yl)oxy]phenyl}amino)-8,9-dihydro-7H-pyrimido[4,5-b]azepin-6-yl]carbonyl}piperazin-1-yl)ethanol

Using 4-({3-methyl-4-[(6-methylpyridin-3-yl)oxy]phenyl}amino)-8,9-dihydro-7H-pyrimido[4,5-b]azepine-6-carboxylic acid (50 mg), 2-piperazin-1-ylethanol (23 mg), 1-ethyl-3-(3-dimethylaminopropyl)carbodiimide hydrochloride (48 mg), 1-hydroxybenzotriazole monohydrate (38 mg), triethylamine (0.10 mL) and N,N-dimethylformamide (5.0 mL), a similar reaction as in Example 136 was carried out to give the title compound (24 mg) as colorless crystals.

¹H-NMR (CDCl₃) δ: 2.34 (3H, s), 2.44-2.52 (7H, m), 2.59 (2H, t, J=5.4 Hz), 2.78 (2H, t, J=4.2 Hz), 3.52-3.71 (8H, m), 5.57-5.64 (1H, m), 6.45 (1H, s), 6.54-6.63 (1H, m), 6.86 (1H, d, J=8.7 Hz), 7.06-7.12 (2H, m), 7.15-7.27 (2H, m), 7.32 (1H, d, J=3.0 Hz), 8.11 (1H, s), 8.24 (1H, d, J=2.4 Hz).

### Example 153

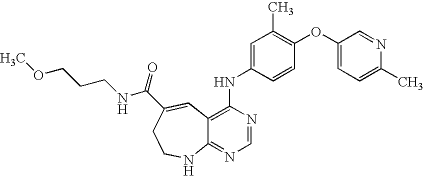

### Production of N-(3-methoxypropyl)-4-({3-methyl-4-[(6-methylpyridin-3-yl)oxy]phenyl}amino)-8,9-dihydro-7H-pyrimido[4,5-b]azepine-6-carboxamide

Using 4-({3-methyl-4-[(6-methylpyridin-3-yl)oxy]phenyl}amino)-8,9-dihydro-7H-pyrimido[4,5-b]azepine-6-carboxylic acid (50 mg), 3-methoxy-1-propylamine (21 mg), 1-ethyl-3-(3-dimethylaminopropyl)carbodiimide hydrochloride (48 mg), 1-hydroxybenzotriazole monohydrate (38 mg), triethylamine (0.10 mL) and N,N-dimethylformamide (5.0 mL), a similar reaction as in Example 136 was carried out to give the title compound (21 mg) as colorless crystals.

¹H-NMR (CDCl₃) δ: 1.80-1.91 (2H, m), 2.23 (3H, s), 2.52 (3H, s), 2.78 (2H, t, J=4.7 Hz), 3.35 (3H, s), 3.46-3.58 (6H, m), 5.57-5.65 (1H, m), 6.58-6.67 (1H, m), 6.85 (1H, d, J=8.4 Hz), 6.93 (1H, s), 7.04-7.11 (2H, m), 7.21-7.27 (1H, m), 7.35 (1H, d, J=2.7 Hz), 7.43 (1H, s), 8.08 (1H, s), 8.25 (1H, dd, J=2.4, 1.2 Hz).

### Example 154

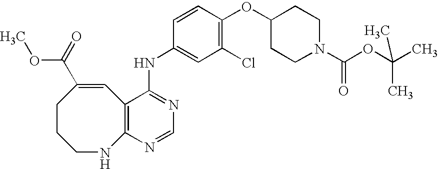

### Production of methyl (5E)-4-[(4-{[1-(tert-butoxycarbonyl)piperidin-4-yl]oxy}-3-chlorophenyl)amino]-7,8,9,10-tetrahydropyrimido[4,5-b]azocine-6-carboxylate

### (i) Production of methyl 5-[(6-chloro-5-formylpyrimidin-4-yl)(4-methoxybenzyl)amino]pentanoate

Using 4,6-dichloro-5-formylpyrimidine (2.0 g), methyl 5-[(4-methoxybenzyl)amino]pentanoate hydrochloride (3.90 g), potassium phosphate (5.76 g) and acetonitrile (30 mL), a similar reaction as in Example 1 (i) was carried out to give the title compound (3.72 g) as a pale-yellow oil.

¹H-NMR (CDCl₃) δ: 1.49-1.70 (4H, m), 2.30 (2H, t, J=7.1 Hz), 3.58 (2H, t, J=7.1 Hz), 3.66 (3H, s), 3.79 (3H, s), 4.58 (2H, s), 6.83 (2H, d, J=8.7 Hz), 7.01 (2H, d, J=8.7 Hz), 8.38 (1H, s), 10.19 (1H, s).

### (ii) Production of tert-butyl 4-[2-chloro-4-({5-formyl-6-[(4-methoxybenzyl)(5-methoxy-5-oxopentyl)amino]pyrimidin-4-yl}amino)phenoxy]piperidine-1-carboxylate

Using methyl 5-[(6-chloro-5-formylpyrimidin-4-yl)(4-methoxybenzyl)amino]pentanoate (520 mg), tert-butyl 4-(4-amino-2-chlorophenoxy)piperidine-1-carboxylate (434 mg), sodium carbonate (141 mg) and N,N-dimethylformamide (5.0 mL), a similar reaction as in Example 100 (i) was carried out to give the title compound (505 mg) as pale-yellow amorphous.

¹H-NMR (CDCl₃) δ: 1.47 (9H, s), 1.52-1.99 (8H, m), 2.33 (2H, t, J=7.1 Hz), 3.36-3.55 (4H, m), 3.62-3.74 (5H, m), 3.81 (3H, s), 4.42-4.53 (1H, m), 4.78 (2H, s), 6.89 (2H, d, J=8.7 Hz), 6.95 (1H, d, J=9.0 Hz), 7.16 (2H, d, J=8.7 Hz), 7.41 (1H, dd, J=9.0, 3.0 Hz), 7.79 (1H, d, J=3.0 Hz), 8.28 (1H, s), 9.78 (1H, s), 11.02 (1H, s).

### (iii) Production of methyl (5E)-4-[(4-{[1-(tert-butoxycarbonyl)piperidin-4-yl]oxy}-3-chlorophenyl)amino]-7,8,9,10-tetrahydropyrimido[4,5-b]azocine-6-carboxylate

Using tert-butyl 4-[2-chloro-4-({5-formyl-6-[(4-methoxybenzyl)(5-methoxy-5-oxopentyl)amino]pyrimidin-4-yl}amino)phenoxy]piperidine-1-carboxylate (505 mg), 28% sodium methoxide-methanol solution (0.43 g) and dimethyl carbonate (10 mL), a similar reaction as in Example 132 (ii) was carried out to give methyl (5E)-4-[(4-{[1-(tert-butoxycarbonyl)piperidin-4-yl]oxy}-3-chlorophenyl)amino]-10-(4-methoxybenzyl)-7,8,9,10-tetrahydropyrimido[4,5-b]azocine-6-carboxylate (316 mg) as yellow amorphous. Using methyl (5E)-4-[(4-{[1-(tert-butoxycarbonyl)piperidin-4-yl]oxy}-3-chlorophenyl)amino]-10-(4-methoxybenzyl)-7,8,9,10-tetrahydropyrimido[4,5-b]azocine-6-carboxylate (316 mg), trifluoroacetic acid (5.0 mL), 1,2-dichloroethane (5.0 mL), tetrahydrofuran (10 mL), triethylamine (1.3 mL) and di-tert-butyl dicarbonate (0.11 mL), a similar reaction as in Example 133 was carried out to give the title compound (181 mg) as colorless crystals.

¹H-NMR (CDCl₃) δ: 1.47 (9H, s), 1.50-1.69 (2H, m), 1.78-1.93 (4H, m), 2.64-2.85 (2H, m), 3.34-3.71 (6H, m), 3.82 (3H, s), 4.42-4.50 (1H, m), 5.18-5.25 (1H, m), 6.25 (1H, s), 6.93 (1H, d, J=9.0 Hz), 7.20-7.28 (1H, m), 7.52 (1H, d, J=2.7 Hz), 7.58 (1H, s), 8.07 (1H, s).

### Example 155

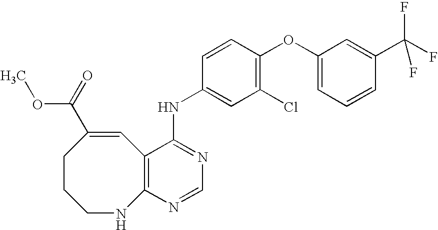

### Production of methyl (5E)-4-({3-chloro-4-[3-(trifluoromethyl)phenoxy]phenyl}amino)-7,8,9,10-tetrahydropyrimido[4,5-b]azocine-6-carboxylate

### (i) Production of methyl (5E)-4-methoxy-7,8,9,10-tetrahydropyrimido[4,5-b]azocine-6-carboxylate

To a solution of methyl 5-[(6-chloro-5-formylpyrimidin-4-yl)(4-methoxybenzyl)amino]pentanoate (2.60 g) in dimethyl carbonate (26 mL) was added a solution of sodium methoxide in methanol (28%, 3.84 g) at room temperature, and the mixture was stirred at 50° C. for 24 hr. The mixture was cooled to 0° C., neutralized with 1N hydrochloric acid and extracted with ethyl acetate. The organic layer was washed with saturated brine, dried over anhydrous magnesium sulfate and concentrated under reduced pressure. The residue was separated and purified by column chromatography (eluent, hexane:ethyl acetate=9:1→7:3→3:2) to give methyl (5E)-4-methoxy-10-(4-methoxybenzyl)-7,8,9,10-tetrahydropyrimido[4,5-b]azocine-6-carboxylate (2.1 g) as colorless amorphous. Using methyl (5E)-4-methoxy-10-(4-methoxybenzyl)-7,8,9,10-tetrahydropyrimido[4,5-b]azocine-6-carboxylate (2.1 g), trifluoroacetic acid (10 mL) and 1,2-dichloroethane (20 mL), a similar reaction as in Example 132 (iii) was carried out to give the title compound (1.35 g) as colorless crystals.

¹H-NMR (CDCl₃) δ: 1.33-1.57 (2H, m), 2.60-2.83 (2H, m), 3.35-3.76 (2H, m), 3.79 (3H, s), 3.96 (3H, s), 5.18-5.29 (1H, s), 7.85 (1H, s), 8.09 (1H, s).

### (ii) Production of methyl (5E)-4-chloro-7,8,9,10-tetrahydropyrimido[4,5-b]azocine-6-carboxylate

A solution of methyl (5E)-4-methoxy-7,8,9,10-tetrahydropyrimido[4,5-b]azocine-6-carboxylate (1.0 g) in phosphoryl chloride (10 g) was stirred at 100° C. for 3 days. Phosphoryl chloride was evaporated under reduced pressure. Ethyl acetate was added to the residue, and the mixture was neutralized with aqueous sodium bicarbonate solution and potassium carbonate and extracted with ethyl acetate. The organic layer was washed with saturated brine, dried over anhydrous magnesium sulfate and concentrated under reduced pressure. The residue was separated and purified by column chromatography (eluent, hexane:ethyl acetate=4:1→3:2) to give the title compound (659 mg) as colorless crystals.

¹H-NMR (CDCl₃) δ: 1.06-1.63 (2H, m), 2.21-2.41 (1H, m), 2.61-2.84 (1H, m), 3.08-3.37 (1H, m), 3.42-3.66 (1H, m), 3.74 (3H, s), 7.73 (1H, s), 7.91 (1H, t, J=6.9 Hz), 8.12 (1H, s).

### (iii) Production of methyl (5E)-4-({3-chloro-4-[3-(trifluoromethyl)phenoxy]phenyl}amino)-7,8,9,10-tetrahydropyrimido[4,5-b]azocine-6-carboxylate

The mixture of methyl (5E)-4-chloro-7,8,9,10-tetrahydropyrimido[4,5-b]azocine-6-carboxylate (100 mg), 3-chloro-4-[3-(trifluoromethyl)phenoxy]aniline (112 mg), pyridine hydrochloride (catalytic amount) and isopropyl alcohol (2 mL) was stirred at 90° C. for 4 hr. Aqueous sodium bicarbonate solution was added to the reaction mixture and the mixture was extracted with ethyl acetate. The organic layer was washed with saturated brine, dried over anhydrous magnesium sulfate and concentrated under reduced pressure. The residue was separated and purified by column chromatography (eluent, hexane:ethyl acetate=1:1→ethyl acetate) to give the title compound (88 mg) as pale-yellow crystals.

¹H-NMR (CDCl₃) δ: 1.23-1.43 (2H, m), 2.50-2.71 (2H, m), 3.22-3.41 (2H, m), 3.74 (3H, s), 7.12 (1H, t, J=6.9 Hz), 7.17-7.23 (3H, m), 7.44-7.47 (1H, m), 7.54 (1H, dd, J=9.0, 2.7 Hz), 7.57-7.63 (1H, m), 7.70 (1H, s), 7.79 (1H, d, J=2.7 Hz), 7.96 (1H, s), 8.55 (1H, s).

### Example 156

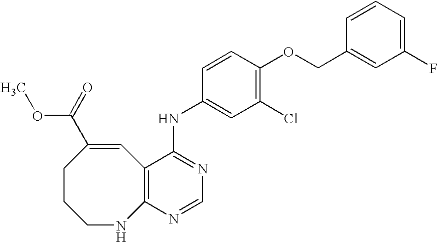

### Production of methyl (5E)-4-({3-chloro-4-[(3-fluorobenzyl)oxy]phenyl}amino)-7,8,9,10-tetrahydropyrimido[4,5-b]azocine-6-carboxylate

A mixture of methyl (5E)-4-chloro-7,8,9,10-tetrahydropyrimido[4,5-b]azocine-6-carboxylate (100 mg), 3-chloro-4-[(3-fluorobenzyl)oxy]aniline (119 mg) and pyridinium chloride (catalytic amount) and isopropyl alcohol (2.0 mL) was stirred at 90° C. for 4 hr, Aqueous sodium bicarbonate solution was added to the reaction mixture, and the mixture was extracted with ethyl acetate. The organic layer was washed with saturated brine, dried over anhydrous magnesium sulfate and concentrated under reduced pressure. The residue was separated and purified by column chromatography (eluent, hexane:ethyl acetate=1:1→ethyl acetate) to give the title compound (71 mg) as pale-yellow crystals.

¹H-NMR (DMSO-d₆) δ: 1.20-1.40 (2H, m), 2.51-2.66 (2H, m), 3.15-3.41 (2H, m), 3.73 (3H, s), 5.20 (2H, s), 7.00 (1H, t, J=7.1 Hz), 7.11-7.20 (2H, m), 7.24-7.36 (3H, m), 7.42-7.49 (1H, m), 7.56 (1H, d, J=2.7 Hz), 7.66 (1H, s), 7.87 (1H, s), 8.30 (1H, s).

### Example 157

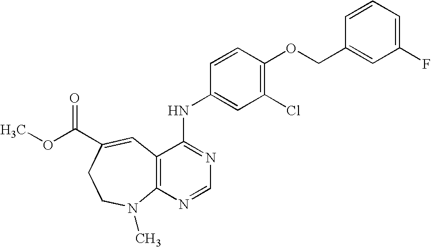

### Production of methyl 4-({3-chloro-4-[(3-fluorobenzyl)oxy]phenyl}amino)-9-methyl-8,9-dihydro-7H-pyrimido[4,5-b]azepine-6-carboxylate

### (i) Production of methyl 4-[(6-chloro-5-formylpyrimidin-4-yl)(methyl)amino]butanoate

To a solution of 4,6-dichloro-5-formylpyrimidine (1.0 g) and 4-(methylamino)butanoic acid hydrochloride (0.87 g) in N,N-dimethylformamide (10 mL) was added sodium carbonate (1.80 g) at room temperature. The mixture was stirred at room temperature for 1 hr. Methyl iodide (0.42 mL) and sodium carbonate (0.72 g) were added to the reaction mixture, and the mixture was further stirred at room temperature for 14 hr. Water was added to reaction mixture, and the mixture was extracted with ethyl acetate. The organic layer was washed with water and saturated brine, dried over anhydrous magnesium sulfate and concentrated under reduced pressure. The residue was separated and purified by column chromatography (eluent, hexane:ethyl acetate=7:3→1:1) to give the title compound (665 mg) as a white solid.

¹H-NMR (CDCl₃) δ: 1.96-2.06 (2H, m), 2.37 (2H, t, J=7.2 Hz), 2.88 (3H, s), 3.67 (3H, s), 3.79 (2H, t, J=257.2 Hz), 8.31 (1H, s), 10.35 (1H, s).

### (ii) Production of methyl 4-[[6-({3-chloro-4-[(3-fluorobenzyl)oxy]phenyl}amino)-5-formylpyrimidin-4-yl](methyl)amino]butanoate

Using methyl 4-[(6-chloro-5-formylpyrimidin-4-yl)(methyl)amino]butanoate (0.50 g), 3-chloro-4-[(3-fluorobenzyl)oxy]aniline (0.463 g), potassium carbonate (0.26 g) and N,N-dimethylformamide (6.0 mL), a similar reaction as in Example 100 (i) was carried out to give the title compound (787 mg) as pale-yellow amorphous.

¹H-NMR (CDCl₃) δ: 2.00-2.10 (2H, m), 2.38 (2H, t, J=7.4 Hz), 3.29 (3H, s), 3.68 (3H, s), 3.71 (2H, t, J=7.5 Hz), 5.13 (2H, s), 6.91 (1H, d, J=8.7 Hz), 6.97-7.03 (1H, m), 7.17-7.25 (3H, m), 7.28-7.41 (2H, m), 7.79 (1H, d, J=2.4 Hz), 8.19 (1H, s), 9.85 (1H, s).

### (iii) Production of methyl 4-({3-chloro-4-[(3-fluorobenzyl)oxy]phenyl}amino)-9-methyl-8,9-dihydro-7H-pyrimido[4,5-b]azepine-6-carboxylate

Using methyl 4-[[6-({3-chloro-4-[(3-fluorobenzyl)oxy]phenyl}amino)-5-formylpyrimidin-4-yl](methyl)amino]butanoate (730 mg), 28% sodium methoxide-methanol solution (0.72 g) and dimethyl carbonate (10 mL), a similar reaction as in Example 100

### (ii) was carried out to give the title compound (494 mg) as yellow crystals

¹H-NMR (CDCl₃) δ: 2.84 (2H, t, J=4.5 Hz), 3.24 (3H, s), 3.44 (2H, t, J=4.5 Hz), 3.79 (3H, s), 5.14 (2H, s), 6.67 (1H, s), 6.92 (1H, d, J=8.7 Hz), 6.97-7.06 (1H, m), 7.17-7.28 (3H, m), 7.32-7.39 (1H, m), 7.56 (1H, d, J=2.4 Hz), 7.64 (1H, s), 8.17 (1H, s).

### Example 158

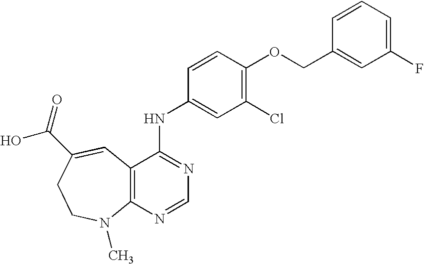

### Production of 4-({3-chloro-4-[(3-fluorobenzyl)oxy]phenyl}amino)-9-methyl-8,9-dihydro-7H-pyrimido[4,5-b]azepine-6-carboxylic acid

To a solution of methyl 4-({3-chloro-4-[(3-fluorobenzyl)oxy]phenyl}amino)-9-methyl-8,9-dihydro-7H-pyrimido[4,5-b]azepine-6-carboxylate (200 mg) in tetrahydrofuran (5.0 mL) and ethanol (5.0 mL) was added 1N aqueous sodium hydroxide solution (1.5 mL) at room temperature. The mixture was stirred at room temperature for 6 hr. 1N Hydrochloric acid (1.5 mL) was added to the mixture and the mixture was concentrated under reduced pressure. Water was added to the residue, and the mixture was extracted with ethyl acetate. The organic layer was washed with saturated brine, dried over anhydrous magnesium sulfate and concentrated under reduced pressure. The precipitated crystals were collected by filtration. The crystals were washed with hexane to give the title compound (186 mg) as yellow crystals.

¹H-NMR (CDCl₃) δ: 2.78-2.86 (2H, m), 3.25 (3H, s), 3.43-3.49 (2H, m), 5.11 (2H, s), 6.88-7.05 (3H, m), 7.16-7.41 (4H, m), 7.53 (1H, d, J=2.7 Hz), 7.71 (1H, s), 8.15 (1H, s).

### Example 159

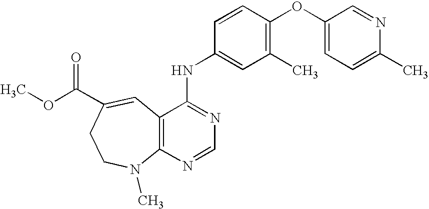

### Production of methyl 9-methyl-4-({3-methyl-4-[(6-methylpyridin-3-yl)oxy]phenyl}amino)-8,9-dihydro-7H-pyrimido[4,5-b]azepine-6-carboxylate

### (i) Production of methyl 4-[[5-formyl-6-({3-methyl-4-[(6-methylpyridin-3-yl)oxy]phenyl}amino)pyrimidin-4-yl](methyl)amino]butanoate

Using methyl 4-[(6-chloro-5-formylpyrimidin-4-yl)(methyl)amino]butanoate (0.50 g), 3-methyl-4-[(6-methylpyridin-3-yl)oxy]aniline (0.414 g), potassium carbonate (0.26 g) and N,N-dimethylformamide (6.0 mL), a similar reaction as in Example 100 (i) was carried out to give the title compound (751 mg) as pale-yellow amorphous.

¹H-NMR (CDCl₃) δ: 2.01-2.11 (2H, m), 2.25 (3H, s), 2.39 (2H, t, J=7.1 Hz), 2.52 (3H, s), 3.30 (3H, s), 3.69 (3H, s), 3.72 (2H, t, J=7.2 Hz), 6.87 (1H, d, J=8.7 Hz), 7.08-7.09 (2H, m), 7.45-7.52 (2H, m), 8.22 (1H, s), 8.25-8.26 (1H, m), 9.88 (1H, s).

### (ii) Production of methyl 9-methyl-4-({3-methyl-4-[(6-methylpyridin-3-yl)oxy]phenyl}amino)-8,9-dihydro-7H-pyrimido[4,5-b]azepine-6-carboxylate

Using methyl 4-[[5-formyl-6-({3-methyl-4-[(6-methylpyridin-3-yl)oxy]phenyl}amino)pyrimidin-4-yl](methyl)amino]butanoate (751 mg), 28% sodium methoxide-methanol solution (0.80 g) and dimethyl carbonate (10 mL), a similar reaction as in Example 100 (ii) was carried out to give the title compound (516 mg) as pale-yellow crystals.

¹H-NMR (CDCl₃) δ: 2.24 (3H, s), 2.52 (3H, s), 2.85 (2H, t, J=4.6 Hz), 3.24 (3H, s), 3.44 (2H, t, J=4.6 Hz), 3.79 (3H, s), 6.72 (1H, s), 6.86 (1H, d, J=9.0 Hz), 7.04-7.11 (2H, m), 7.22-7.29 (1H, m), 7.33-7.36 (1H, m), 7.66 (1H, s), 8.18 (1H, s), 8.24-8.26 (1H, m).

### Example 160

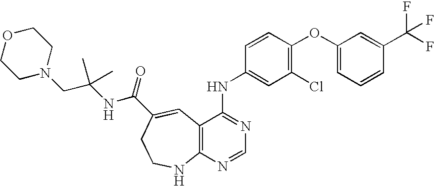

### Production of 4-({3-chloro-4-[3-(trifluoromethyl)phenoxy]phenyl}amino)-N-(1,1-dimethyl-2-morpholin-4-ylethyl)-8,9-dihydro-7H-pyrimido[4,5-b]azepine-6-carboxamide

Using 4-({3-chloro-4-[3-(trifluoromethyl)phenoxy]phenyl}amino)-8,9-dihydro-7H-pyrimido[4,5-b]azepine-6-carboxylic acid (131 mg), 2-methyl-1-(morpholin-4-yl)-2-propylamine (67.5 mg), 1-hydroxybenzotriazole (56.8 mg), triethylamine (0.1 mL), 1-ethyl-3-(3-dimethylaminopropyl)carbodiimide hydrochloride (86.8 mg), tetrahydrofuran (0.7 mL) and N,N-dimethylformamide (0.7 mL), a similar reaction as in Example 110 was carried out to give the title compound (139 mg) as a white powder.

¹H-NMR (DMSO-d₆) δ: 1.29 (6H, s), 2.41-2.48 (4H, m), 2.54 (2H, s), 2.58-2.69 (2H, m), 3.28-3.40 (2H, m), 3.41-3.53 (4H, m), 7.00 (1H, s), 7.13-7.23 (2H, m), 7.26 (1H, d, J=8.9 Hz), 7.30 (1H, s), 7.45 (1H, d, J= 7.5 Hz), 7.54-7.63 (2H, m), 7.67 (1H, t, J=4.9 Hz), 7.92 (1H, d, J=2.5 Hz), 8.00 (1H, s), 9.24 (1H, s).

### Example 161

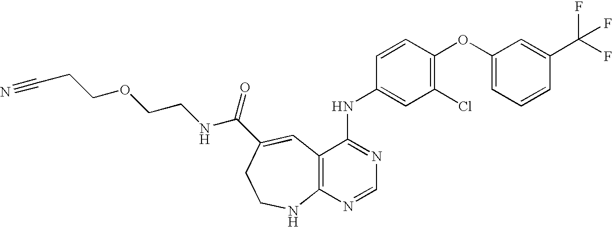

### Production of 4-({3-chloro-4-[3-(trifluoromethyl)phenoxy]phenyl}amino)-N-[2-(2-cyanoethoxy)ethyl]-8,9-dihydro-7H-pyrimido[4,5-b]azepine-6-carboxamide

### (i) Production of tert-butyl [2-(2-cyanoethoxy)ethyl]carbamate

To a solution of tert-butyl [2-(2-hydroxyethoxy)ethyl]carbamate (499 mg) in tetrahydrofuran (5 mL) were added triphenylphosphine (957 mg) and 40% diethyl azodicarboxylate-toluene solution (1.7 mL), and the mixture was stirred at 0° C. for 10 min. 2-Hydroxy-2-methylpropanenitrile (0.35 mL) was added to the reaction mixture, and the mixture was stirred at room temperature for 6 hr. The reaction mixture was concentrated under reduced pressure, and the residue was separated and purified by silica gel column chromatography (eluent, hexane:ethyl acetate=67:33→20:80) and basic silica gel column chromatography (eluent, hexane:ethyl acetate=80:20→33:67) to give the title compound (292 mg) as a colorless oil.

¹H-NMR (CDCl₃) δ: 1.45 (9H, s), 2.61 (2H, t, J=6.3 Hz), 3.34 (2H, q, J=5 Hz), 3.56 (2H, t, J=5 Hz), 3.68 (2H, t, J=6.3 Hz), 4.87 (1H, br s).

### (ii) Production of 3-(2-aminoethoxy)propylnitrile hydrochloride

Using tert-butyl [2-(2-cyanoethoxy)ethyl]carbamate (289 mg), 6N hydrochloric acid (2.0 mL) and tetrahydrofuran (10 mL), a similar reaction as in Example 127 (vi) was carried out to give the title compound (214 mg) as a white solid.

¹H-NMR (DMSO-d₆) δ: 2.80 (2H, t, J=5.9 Hz), 2.97 (2H, t, J=5.3 Hz), 3.56-3.80 (4H, m), 8.05 (3H, br s).

### (iii) Production of 4-({3-chloro-4-[3-(trifluoromethyl)phenoxy]phenyl}amino)-N-[2-(2-cyanoethoxy)ethyl]-8,9-dihydro-7H-pyrimido[4,5-b]azepine-6-carboxamide

Using 4-({3-chloro-4-[3-(trifluoromethyl)phenoxy]phenyl}amino)-8,9-dihydro-7H-pyrimido[4,5-b]azepine-6-carboxylic acid (129 mg), 3-(2-aminoethoxy)propylnitrile hydrochloride (62.6 mg), 1-hydroxybenzotriazole (55.0 mg), triethylamine (0.3 mL), 1-ethyl-3-(3-dimethylaminopropyl)carbodiimide hydrochloride (7-9.6 mg), tetrahydrofuran (0.7 mL) and N,N-dimethylformamide (0.7 mL), a similar reaction as in Example 110 was carried out to give the title compound (96.1 mg) as a white powder.

¹H-NMR (DMSO-d₆) δ: 2.65-2.78 (4H, m), 3.29-3.40 (4H, m), 3.53 (2H, t, J=6.0 Hz), 3.61 (2H, t, J=6.0 Hz), 7.15-7.23 (3H, m), 7.26 (1H, d, J=8.9 Hz), 7.46 (1H, d, J=7.7 Hz), 7.55-7.65 (2H, m), 7.68 (1H, t, J=4.7 Hz), 7.90 (1H, d, J=2.6 Hz), 7.98 (1H, s), 8.06 (1H, t, J=5.7 Hz), 9.14 (1H, s).

### Example 162

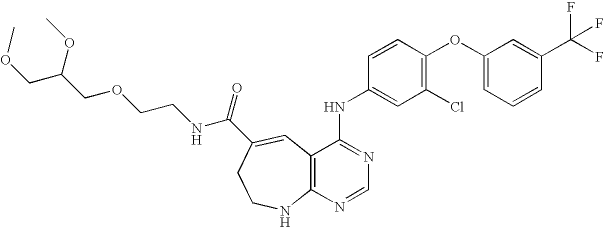

### Production of 4-({3-chloro-4-[3-(trifluoromethyl)phenoxy]phenyl}amino)-N-[2-(2,3-dimethoxypropoxy)ethyl]-8,9-dihydro-7H-pyrimido[4,5-b]azepine-6-carboxamide

### (i) Production of 4-{[2-(benzyloxy)ethoxy]methyl}-2,2-dimethyl-1,3-dioxolane

Using [(2-bromoethoxy)methyl]benzene (3 mL), (2,2-dimethyl-1,3-dioxolan-4-yl)methanol (2.80 g), tetra-n-butylammonium hydrogen sulfate (667 mg), toluene (40 mL) and 50% aqueous sodium hydroxide solution (10 mL), a similar reaction as in Example 124 (i) was carried out to give the title compound (3.65 g) as a colorless oil.

¹H-NMR (CDCl₃) δ: 1.36 (3H, s), 1.42 (3H, s), 3.47-3.78 (7H, m), 4.05 (1H, dd, J=8.3, 6.3 Hz), 4.56 (2H, s), 7.19-7.43 (5H, m).

### (ii) Production of 2-[(2,2-dimethyl-1,3-dioxolan-4-yl)methoxy]ethanol

Using 4-{[2-(benzyloxy)ethoxy]methyl}-2,2-dimethyl-1,3-dioxolane (3.64 g), 10% palladium-carbon (372 mg) and methanol (40 mL), a similar reaction as in Example 123 (ii) was carried out to give the title compound (2.32 g) as a colorless oil.

¹H-NMR (CDCl₃) δ: 1.37 (3H, s), 1.44 (3H, s), 2.21 (1H, t, J=5.5 Hz), 3.51-3.81 (7H, m), 4.06 (1H, dd, J=8.3, 6.6 Hz), 4.25-4.37 (1H, m).

### (iii) Production of 4-[(2-azidoethoxy)methyl]-2,2-dimethyl-1,3-dioxolane

To a solution of 2-[(2,2-dimethyl-1,3-dioxolan-4-yl)methoxy]ethanol (1.30 g) in ethyl acetate (25 mL) were added triethylamine (1.6 mL) and methanesulfonyl chloride (0.65 mL) under ice-cooling, and the mixture was stirred at 0° C. for 1 hr. Water was added to the reaction mixture, and the mixture was extracted with ethyl acetate. The organic layer was washed with saturated brine, dried over anhydrous magnesium sulfate and concentrated under reduced pressure. The residue was dissolved in N,N-dimethylformamide (20 mL). Sodium azide (629 mg) was added to the solution and the mixture was stirred at 50° C. for 16 hr. The reaction mixture was concentrated under reduced pressure. Water was added to the residue, and the mixture was extracted with ethyl acetate. The organic layer was washed with saturated brine, dried over anhydrous magnesium sulfate and concentrated under reduced pressure. The residue was separated and purified by silica gel column chromatography (eluent, hexane:ethyl acetate=90:10→50:50) to give the title compound (1.32 g) as a colorless oil.

¹H-NMR (CDCl₃) δ: 1.37 (3H, s), 1.43 (3H, s), 3.30-3.47 (2H, m), 3.48-3.84 (5H, m), 4.07 (1H, dd, J=8.4, 6.5 Hz), 4.22-4.36 (1H, m).

### (iv) Production of 3-(2-azidoethoxy)propane-1,2-diol

To a solution of 4-[(2-azidoethoxy)methyl]-2,2-dimethyl-1,3-dioxolane (1.31 g) in methanol (20 mL) was added pyridinium p-toluenesulfonate (1.63 g), and the mixture was stirred at room temperature for 17.5 hr. Pyridinium p-toluenesulfonate (802 mg) was added to the mixture, and the mixture was stirred at room temperature for 8 hr. Saturated aqueous sodium hydrogencarbonate solution was added to the reaction mixture, and the mixture was extracted with ethyl acetate. The organic layer was dried over anhydrous magnesium sulfate and concentrated under reduced pressure. The residue was separated and purified by silica gel column chromatography (eluent, hexane:ethyl acetate=67:33→0:100) to give the title compound (707 mg) as a colorless oil.

¹H-NMR (CDCl₃) δ: 2.05 (1H, t, J=5.5 Hz), 2.60 (1H, d, J=4.5 Hz), 3.28-3.51 (2H, m), 3.51-3.82 (6H, m), 3.81-4.03 (1H, m).

### (v) Production of tert-butyl [2-(2,3-dihydroxypropoxy)ethyl]carbamate

Using 3-(2-azidoethoxy)propane-1,2-diol (701 mg), triphenylphosphine (1.27 g), tetrahydrofuran (7 mL), water (0.7 mL), di-tert-butyl dicarbonate (1.3 mL), triethylamine (1 mL) and methanol (10 mL), a similar reaction as in Example 127 (iv) was carried out to give the title compound (879 mg) as a colorless oil.

¹H-NMR (CDCl₃) δ: 1.45 (9H, s), 2.34 (1H, br s), 2.86 (1H, br s), 3.33 (2H, q, J=5.0 Hz), 3.46-3.80 (6H, m), 3.79-3.97 (1H, m), 4.90 (1H, br s).

### (vi) Production of tert-butyl [2-(2,3-dimethoxypropoxy)ethyl]carbamate

Using tert-butyl [2-(2,3-dihydroxypropoxy)ethyl]carbamate (670 mg), 60% sodium hydride (dispersion in mineral oil, 289 mg), methyl iodide (0.4 mL) and tetrahydrofuran (28 mL), a similar reaction as in Example 128 (i) was carried out to give the title compound (335 mg) as a pale-yellow oil.

¹H-NMR (CDCl₃) δ: 1.45 (9H, s), 3.24-3.36 (2H, m), 3.38 (3H, s), 3.42-3.66 (7H, m), 3.46 (3H, s), 4.96 (1H, br s).

### (vii) Production of 2-(2,3-dimethoxypropoxy)ethyl hydrochloride

Using tert-butyl [2-(2,3-dimethoxypropoxy)ethyl]carbamate (331 mg), 6N hydrochloric acid (1 mL) and ethanol (3 mL), a similar reaction as in Example 127 (vi) was carried out to give the title compound (260 mg) as a colorless oil.

¹H-NMR (DMSO-d₆) δ: 2.94 (2H, t, J=5.4 Hz), 3.26 (3H, s), 3.29-3.53 (5H, m), 3.32 (3H, s), 3.60 (2H, t, J=5.4 Hz), 8.05 (3H, br s).

### (viii) Production of 4-({3-chloro-4-[3-(trifluoromethyl)phenoxy]phenyl}amino)-N-[2-(2,3-dimethoxypropoxy)ethyl]-8,9-dihydro-7H-pyrimido[4,5-b]azepine-6-carboxamide

Using 4-({3-chloro-4-[3-(trifluoromethyl)phenoxy]phenyl}amino)-8,9-dihydro-7H-pyrimido[4,5-b]azepine-6-carboxylic acid (130 mg), 2-(2,3-dimethoxypropoxy)ethylamine hydrochloride (82.9 mg), 1-hydroxybenzotriazole (56.7 mg), triethylamine (0.3 mL), 1-ethyl-3-(3-dimethylaminopropyl)carbodiimide hydrochloride (80.9 mg), tetrahydrofuran (0.7 mL) and N,N-dimethylformamide (0.7 mL), a similar reaction as in Example 110 was carried out to give the title compound (118 mg) as a white powder.

¹H-NMR (DMSO-d₆) δ: 2.63-2.73 (2H, m), 3.20 (3H, s), 3.28 (3H, s), 3.29-3.53 (11H, m), 7.13-7.22 (3H, m), 7.26 (1H, d, J=8.9 Hz), 7.46 (1H, d, J=7.7 Hz), 7.55-7.65 (2H, m), 7.68 (1H, t, J=4.6 Hz), 7.88 (1H, d, J=2.6 Hz), 7.98 (1H, s), 8.04 (1H, t, J=5.5 Hz), 9.14 (1H, s).

### Example 163

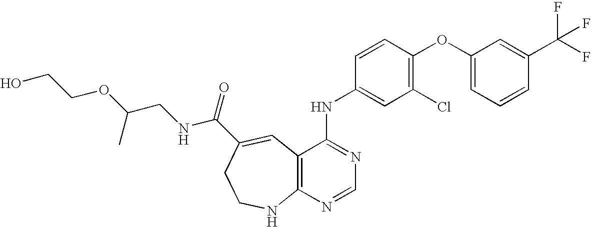

### Production of 4-({3-chloro-4-[3-(trifluoromethyl)phenoxy]phenyl}amino)-N-[2-(2-hydroxyethoxy)propyl]-8,9-dihydro-7H-pyrimido[4,5-b]azepine-6-carboxamide

### (i) Production of 1-(2-[2-(benzyloxy)ethoxy]propoxy)-4-methoxybenzene

Using [(2-bromoethoxy)methyl]benzene (4.01 g), 1-(4-methoxyphenoxy)propan-2-ol (5.05 g), tetra-n-butylammonium hydrogen sulfate (947 mg), toluene (60 mL) and 50% aqueous sodium hydroxide solution (15 mL), a similar reaction as in Example 124 (i) was carried out to give the title compound (2.72 g) as a yellow oil.

¹H-NMR (CDCl₃) δ: 1.28 (3H, d, J=6.2 Hz), 3.60-3.68 (2H, m), 3.74-3.80 (5H, m), 3.80-3.91 (2H, m), 3.92-4.02 (1H, m), 4.58 (2H, s), 6.74-6.93 (4H, m), 7.18-7.44 (5H, m).

### (ii) Production of 2-[2-(benzyloxy)ethoxy]propan-1-ol

To a mixed solution of 1-(2-[2-(benzyloxy)ethoxy]propoxy)-4-methoxybenzene (2.71 g) in acetonitrile (80 mL) and water (20 mL) was added diammonium cerium nitrate (7.04 g) under ice-cooling, and the mixture was stirred at 0° C. for 4 hours. Additional diammonium cerium nitrate (2.36 g) was added to the mixture and the mixture was stirred at room temperature for 2 hr. Saturated aqueous sodium sulfite solution and saturated aqueous sodium hydrogencarbonate solution were added to the reaction mixture, and the mixture was extracted with ethyl acetate. The organic layer was washed with saturated brine, dried over anhydrous magnesium sulfate and concentrated under reduced pressure. The residue was separated and purified by silica gel column chromatography (eluent, hexane:ethyl acetate=80:20→33:67) and basic silica gel column chromatography (eluent, hexane:ethyl acetate=90:10→50:50) to give the title compound (1.53 g) as a pale-yellow oil.

¹H-NMR (CDCl₃) δ: 1.12 (3H, d, J=6.2 Hz), 2.65 (1H, dd, j=8.6, 4.1 Hz), 3.37-3.52 (1H, m), 3.51-3.70 (5H, m), 3.75-3.91 (1H, m), 4.58 (2H, s), 7.19-7.46 (5H, m).

### (iii) Production of {[2-(2-azido-1-methylethoxy)ethoxy]methyl}benzene

Using 2-[2-(benzyloxy)ethoxy]propan-1-ol (1.52 g), methanesulfonyl chloride (0.65 mL), triethylamine (1.5 mL), ethyl acetate (30 mL), sodium azide (621 mg) and N,N-dimethylformamide (20 mL), a similar reaction as in Example 162 (iii) was carried out to give the title compound (1.59 g) as a colorless oil.

1H-NMR (CDCl₃) δ: 1.20 (3H, d, J=6.3 Hz), 3.10-3.22 (1H, m), 3.22-3.37 (1H, m), 3.56-3.83 (5H, m), 4.58 (2H, s), 7.19-7.45 (5H, m).

### (iv) Production of tert-butyl {2-[2-(benzyloxy)ethoxy]propyl}carbamate

Using {[2-(2-azido-1-methylethoxy)ethoxy]methyl}benzene (1.58 g), triphenylphosphine (1.90 g), tetrahydrofuran (17 mL), water (1.5 mL), di-tert-butyl dicarbonate (2 mL), triethylamine (1.5 mL) and methanol (20 mL), a similar reaction as in Example 127 (iv) was carried out to give the title compound (1.99 g) as a colorless oil.

¹H-NMR (CDCl₃) δ: 1.14 (3H, d, J=6.2 Hz), 1.44 (9H, s), 2.93-3.12 (1H, m), 3.24-3.43 (1H, m), 3.47-3.69 (4H, m), 3.68-3.82 (1H, m), 4.58 (2H, s), 5.14 (1H, br s), 7.20-7.44 (5H, m).

### (v) Production of tert-butyl [2-(2-hydroxyethoxy)propyl]carbamate

Using tert-butyl {2-[2-(benzyloxy)ethoxy]propyl}carbamate (1.98 g), 10% palladium-carbon (302 mg) and methanol (20 mL), a similar reaction as in Example 123 (ii) was carried out to give the title compound (1.38 g) as a colorless oil.

¹H-NMR (CDCl₃) δ: 1.15 (3H, d, J=6.1 Hz), 1.45 (9H, s), 2.13 (1H, t, J=5.9 Hz), 2.93-3.17 (1H, m), 3.24-3.44 (1H, m), 3.43-3.84 (5H, m), 4.90 (1H, br s).

### (vi) Production of 2-(2-amino-1-methylethoxy)ethanol hydrochloride

Using tert-butyl [2-(2-hydroxyethoxy)propyl]carbamate (681 mg), 6N hydrochloric acid (1.5 mL) and tetrahydrofuran (7 mL), a similar reaction as in Example 127 (vi) was carried out to give the title compound (459 mg) as white crystals.

¹H-NMR (DMSO-d₆) δ: 1.11 (3H, d, J=6.1 Hz), 2.71 (1H, dd, J=13, 8.9 Hz), 2.91 (1H, dd, J=13, 3.4 Hz), 3.22-3.44 (2H, m), 3.43-3.79 (4H, m), 7.65 (3H, br s).

### (vii) Production of 4-({3-chloro-4-[3-(trifluoromethyl)phenoxy]phenyl}amino)-N-[2-(2-hydroxyethoxy)propyl]-8,9-dihydro-7H-pyrimido[4,5-b]azepine-6-carboxamide

Using 4-({3-chloro-4-[3-(trifluoromethyl)phenoxy]phenyl}amino)-8,9-dihydro-7H-pyrimido[4,5-b]azepine-6-carboxylic acid (119 mg), 2-(2-amino-1-methylethoxy)ethanol hydrochloride (58.9 mg), 1-hydroxybenzotriazole (51.2 mg), triethylamine (0.35 mL), 1-ethyl-3-(3-dimethylaminopropyl)carbodiimide hydrochloride (73.5 mg), tetrahydrofuran (0.6 mL) and N,N-dimethylformamide (0.6 mL), a similar reaction as in Example 110 was carried out to give the title compound (79.0 mg) as a pale-yellow powder.

¹H-NMR (DMSO-d₆) δ: 1.07 (3H, d, J=6.1 Hz), 2.62-2.77 (2H, m), 3.20 (2H, t, J=5.7 Hz), 3.27-3.39 (2H, m), 3.39-3.60 (5H, m), 4.57 (1H, t, J=5.1 Hz), 7.12-7.22 (3H, m), 7.25 (1H, d, J=9.1 Hz), 7.46 (1H, d, J=7.2 Hz), 7.53-7.65 (2H, m), 7.68 (1H, t, J=4.5 Hz), 7.90 (1H, d, J=1.9 Hz), 7.94-8.05 (2H, m), 9.16 (1H, s).

### Example 164

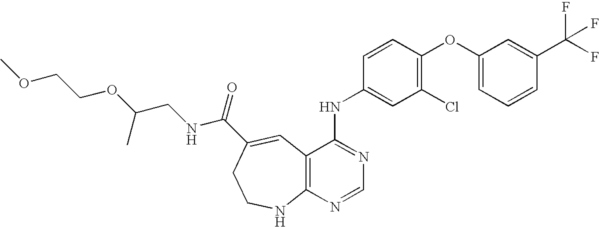

### Production of 4-({3-chloro-4-[3-(trifluoromethyl)phenoxy]phenyl}amino)-N-[2-(2-methoxyethoxy)propyl]-8,9-dihydro-7H-pyrimido[4,5-b]azepine-6-carboxamide

### (i) Production of tert-butyl [2-(2-methoxyethoxy)propyl]carbamate

Using tert-butyl [2-(2-hydroxyethoxy)propyl]carbamate (700 mg), 60% sodium hydride (dispersion in mineral oil, 153 mg), methyl iodide (0.22 mL) and tetrahydrofuran (32 mL), a similar reaction as in Example 128 (i) was carried out to give the title compound (381 mg) as a pale-yellow oil.

¹H-NMR (CDCl₃) δ: 1.14 (3H, d, J=6.1 Hz), 1.44 (9H, s), 2.95-3.07 (1H, m), 3.24-3.38 (1H, m), 3.40 (3H, s), 3.46-3.60 (4H, m), 3.64-3.79 (1H, m), 5.15 (1H, br s).

### (ii) Production of 2-(2-methoxyethoxy)-1-propylamine hydrochloride

Using tert-butyl [2-(2-methoxyethoxy)propyl]carbamate (375 mg), 6N hydrochloric acid (1 mL) and tetrahydrofuran (4 mL), a similar reaction as in Example 127 (vi) was carried out to give the title compound (270 mg) as a colorless oil.

¹H-NMR (DMSO-d₆) δ: 1.11 (3H, d, J=6.4 Hz), 2.61-2.81 (1H, m), 2.79-2.97 (1H, m), 3.25 (3H, s), 3.39-3.55 (3H, m), 3.56-3.80 (2H, m), 7.98 (3H, br s).

### (iii) Production of 4-({3-chloro-4-[3-(trifluoromethyl)phenoxy]phenyl}amino)-N-[2-(2-methoxyethoxy)propyl]-8,9-dihydro-7H-pyrimido[4,5-b]azepine-6-carboxamide

Using 4-({3-chloro-4-[3-(trifluoromethyl)phenoxy]phenyl}amino)-8,9-dihydro-7H-pyrimido[4,5-b]azepine-6-carboxylic acid (119 mg), 2-(2-methoxyethoxy)-1-propylamine hydrochloride (67.8 mg), 1-hydroxybenzotriazole (52.1 mg), triethylamine (0.35 mL), 1-ethyl-3-(3-dimethylaminopropyl)carbodiimide hydrochloride (73.3 mg), tetrahydrofuran (0.6 mL) and N,N-dimethylformamide (0.6 mL), a similar reaction as in Example 110 was carried out to give the title compound (104 mg) as a yellow powder.

¹H-NMR (DMSO-d₆) δ: 1.07 (3H, d, J=6.1 Hz), 2.64-2.75 (2H, m), 3.14-3.2-3 (4H, m), 3.28-3.43 (5H, m), 3.46-3.64 (3H, m), 7.11-7.23 (3H, m), 7.26 (1H, d, J=8.7 Hz), 7.46 (1H, d, J=7.6 Hz), 7.55-7.65 (2H, m), 7.69 (1H, t, J=4.7 Hz), 7.89 (1H, d, J=2.3 Hz), 7.93-8.02 (2H, m), 9.16 (1H, s).

### Example 165

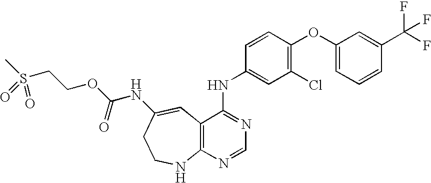

### Production of 2-(methylsulfonyl)ethyl [4-({3-chloro-4-[3-(trifluoromethyl)phenoxy]phenyl}amino)-8,9-dihydro-7H-pyrimido[4,5-b]azepin-6-yl]carbamate

To a solution of 4-({3-chloro-4-[3-(trifluoromethyl)phenoxy]phenyl}amino)-8,9-dihydro-7H-pyrimido[4,5-b]azepine-6-carboxylic acid (100 mg) in tetrahydrofuran (1.5 mL) were successively added triethylamine (0.04 mL) and diphenylphosphoryl azide (0.05 mL), and the mixture was stirred at room temperature for 2 hr. A solution of 2-(methylsulfonyl)ethanol (50.0 mg) in tetrahydrofuran (0.5 mL) was added dropwise to the reaction solution, and the mixture was stirred at 60° C. for 3 hr. Saturated aqueous sodium hydrogen carbonate solution was added to the reaction mixture, and the mixture was extracted with ethyl acetate. The organic layer was washed with saturated brine, dried over anhydrous magnesium sulfate and concentrated under reduced pressure. The residue was separated and purified by silica gel column chromatography (eluent, hexane:ethyl acetate=10:90→0:100→ethyl acetate:methanol=95:5) and basic silica gel column chromatography (eluent, hexane:ethyl acetate=10:90→0:100→ethyl acetate:methanol=90:10), and crystallized from diisopropyl ether-ethyl acetate to give the title compound (15.9 mg) as a pale-yellow powder.

¹H-NMR (DMSO-d₆) δ: 2.58-2.70 (2H, m), 3.07 (3H, s), 3.30-3.43 (2H, m), 3.50 (2H, t, J=5.7 Hz), 4.36 (2H, t, J=5.7 Hz), 6.60 (1H, s), 7.12-7.25 (3H, m), 7.27 (1H, t, J=4.2 Hz), 7.45 (1H, d, J=8.0 Hz), 7.53-7.65 (2H, m), 7.87 (1H, d, J=2.7 Hz), 7.94 (1H, s), 8.54 (1H, t, J=5.7 Hz), 8.72 (1H, s).

### Example 166

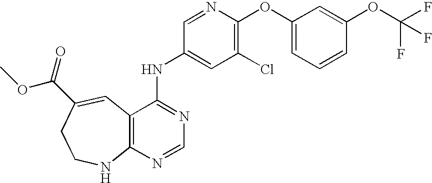

### Production of methyl 4-({5-chloro-6-[3-(trifluoromethoxy)phenoxy]pyridin-3-yl}amino)-8,9-dihydro-7H-pyrimido[4,5-b]azepine-6-carboxylate

### (i) Production of methyl 4-methoxy-9-(4-methoxybenzyl)-8,9-dihydro-7H-pyrimido[4,5-b]azepine-6-carboxylate

To a solution of methyl 4-[(6-chloro-5-formylpyrimidin-4-yl)(4-methoxybenzyl)amino]butanoate (15.4 g) in dimethyl carbonate (150 mL) was added 28% sodium methoxide-methanol solution (23.6 g) at room temperature. The mixture was stirred at room temperature, for 24 hr. Water was added to the reaction mixture, and the mixture was extracted with ethyl acetate. The organic layer was washed with saturated brine, dried over anhydrous magnesium sulfate and concentrated under reduced pressure. The residue was separated and purified by silica gel column chromatography (eluent, hexane:ethyl acetate=3:1→1:1) to give the title compound (12.8 g) as colorless crystals.

¹H-NMR (CDCl₃) δ: 2.66-2.70 (2H, m), 3.28-3.32 (2H, m), 3.78 (3H, s), 3.79 (3H, s), 4.03 (3H, s), 4.88 (2H, s), 6.85 (2H, d, J=8.7 Hz), 7.19 (2H, d, J=8.7 Hz), 8.09 (1H, t, J=1.2 Hz), 8.24 (1H, s).

### (ii) Production of methyl 4-methoxy-8,9-dihydro-7H-pyrimido[4,5-b]azepine-6-carboxylate

To a solution of methyl 4-methoxy-9-(4-methoxybenzyl)-8,9-dihydro-7H-pyrimido[4,5-b]azepine-6-carboxylate (476 mg) in toluene (5.0 mL) was added trifluoroacetic acid (2.5 mL) at room temperature. The mixture was stirred at 70° C. for 20 hr and concentrated under reduced pressure. Aqueous sodium bicarbonate solution was added to the residue and the mixture was extracted with a mixed solvent of ethyl acetate-tetrahydrofuran. The organic layer was washed with saturated brine, dried over anhydrous magnesium sulfate and concentrated under reduced pressure. The precipitated crystals were collected by filtration and washed with diisopropyl ether to give the title compound (288 mg) as colorless crystals.

¹H-NMR (CDCl₃) δ: 2.89 (2H, t, J=4.5 Hz), 3.46 (2H, q, J=4.5 Hz), 3.80 (3H, s), 4.01 (3H, s), 5.79-5.87 (1H, m), 8.04 (1H, d, J=1.2 Hz), 8.12 (1H, s).

### (iii) Production of methyl 4-chloro-8,9-dihydro-7H-pyrimido[4,5-b]azepine-6-carboxylate

A mixture of methyl 4-methoxy-8,9-dihydro-7H-pyrimido[4,5-b]azepine-6-carboxylate (2.55 g) and phosphoryl chloride (26 mL) was stirred at 100° C. for 96 hr. The reaction mixture was concentrated under reduced pressure, and ice was added to the residue at 0° C. The mixture was neutralized with saturated aqueous ammonia. The precipitate was collected by filtration and washed with water to give the title compound (2.40 g) as a pale-yellow powder.

¹H-NMR (CDCl₃) δ: 2.88-2.98 (2H, m), 3.46-3.57 (2H, m), 3.84 (3H, s), 6.18 (1H, br s), 8.14 (1H, s), 8.20 (1H, s).

### (iv) Production of 3-chloro-5-nitro-2-[3-(trifluoromethoxy)phenoxy]pyridine

To a solution of 3-(trifluoromethoxy)phenol (0.93 g) in tetrahydrofuran (10 mL) was added 60% sodium hydride (dispersion in mineral oil, 0.23 g) at 0° C. The mixture was stirred at 0° C. for 1 hr, and 2,3-dichloro-5-nitropyridine (1.0 g) was added thereto. The mixture was stirred at room temperature for 4 hr. Water was added to the reaction mixture, and the mixture was extracted with ethyl acetate. The organic layer was washed with water and saturated brine, dried over anhydrous magnesium sulfate and concentrated under reduced pressure. The residue was separated and purified by silica gel column chromatography (eluent, hexane:ethyl acetate=19:1->3:1) to give the title compound (1.57 g) as a pale-yellow oil.

¹H-NMR (CDCl₃) δ: 7.06-7.22 (3H, m), 7.49 (1H, t, J=8.3 Hz), 8.59 (1H, d, J=2.4 Hz), 8.88 (1H, d, J=2.4 Hz).

### (v) Production of 5-chloro-6-[3-(trifluoromethoxy)phenoxy]pyridin-3-amine

A mixture of 3-chloro-5-nitro-2-[3-(trifluoromethoxy)phenoxy]pyridine (1.57 g), reduced iron (1.31 g), calcium chloride (0.26 g) and 15% water-containing ethanol (50 mL) was heated under reflux for 10 hr. An insoluble material was removed by filtration, and the filtrate was concentrated under reduced pressure. Water was added to the residue, and the mixture was extracted with ethyl acetate. The organic layer was washed with water and saturated brine, dried over anhydrous magnesium sulfate and concentrated under reduced pressure. The residue was separated and purified by silica gel column chromatography (eluent, hexane:ethyl acetate=3:1→2:1) to give the title compound (1.02 g) as a yellow solid.

¹H-NMR (CDCl₃) δ: 3.65 (2H, br s), 6.91-7.02 (3H, m), 7.18 (1H, d, J=2.7 Hz), 7.35 (1H, t, J=8.1 Hz), 7.59 (1H, d, J=2.7 Hz).

### (vi) Production of methyl 4-({5-chloro-6-[3-(trifluoromethoxy)phenoxy]pyridin-3-yl}amino)-8,9-dihydro-7H-pyrimido[4,5-b]azepine-6-carboxylate

A mixture of methyl 4-chloro-8,9-dihydro-7H-pyrimido[4,5-b]azepine-6-carboxylate (100 mg), 5-chloro-6-[3-(trifluoromethoxy)phenoxy]pyridin-3-amine (127 mg) and pyridine hydrochloride (catalytic amount) in isopropyl alcohol (2.0 mL) was stirred at 90° C. for 24 hr. Aqueous sodium bicarbonate solution was added to the reaction mixture, and the mixture was extracted with ethyl acetate. The organic layer was washed with saturated brine, dried over anhydrous magnesium sulfate and concentrated under reduced pressure. The residue was separated and purified by silica gel column chromatography (eluent, hexane:ethyl acetate=1:1→ethyl acetate) to give the title compound (94 mg) as pale-yellow crystals.

¹H-NMR (CDCl₃) δ: 2.91 (2H, t, J=5.1 Hz), 3.48-3.56 (2H, m), 3.82 (3H, s), 5.78-5.85 (1H, m), 6.75 (1H, s), 7.03-7.12 (3H, m), 7.38-7.43 (1H, m), 7.65 (1H, s), 8.05-8.08 (2H, m), 8.19 (1H, d, J=2.7 Hz).

### Example 167

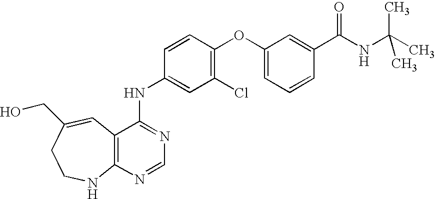

### Production of N-(tert-butyl)-3-(2-chloro-4-{[6-(hydroxymethyl)-8,9-dihydro-7H-pyrimido[4,5-b]azepin-4-yl]amino}phenoxy)benzamide

### (i) Production of 4-[(4-{3-[(tert-butylamino) carbonyl]phenoxy}-3-chlorophenyl)amino]-8,9-dihydro-7H-pyrimido[4,5-b]azepine-6-carboxylic acid

To a solution of methyl 4-[(4-{3-[(tert-butylamino) carbonyl]phenoxy}-3-chlorophenyl)amino]-8,9-dihydro-7H-pyrimido[4,5-b]azepine-6-carboxylate (1.10 g) in tetrahydrofuran (45 mL) and ethanol (45 mL) was added 1N aqueous sodium hydroxide solution (6.0 mL) at room temperature. The mixture was stirred at room temperature for 14 hr, and 1N hydrochloric acid (6.0 mL) was added to the reaction mixture. The solvent was evaporated under reduced pressure, and the precipitated crystals were collected by filtration. The crystals were washed with water to give the title compound (998 mg) as pale-yellow crystals.

¹H-NMR (DMSO-d₆) δ: 1.36 (9H, s), 2.67-2.75 (2H, m), 3.32-3.42 (2H, m), 7.04 (1H, dd, J=7.8, 2.4 Hz), 7.35 (1H, br s), 7.39-7.44 (2H, m), 7.50 (1H, dd, J=8.7, 2.4 Hz), 7.55 (1H, d, J=7.8 Hz), 7.71 (1H, s), 7.80 (1H, d, J=2.4 Hz), 7.82 (1H, s), 7.92-8.09 (2H, m), 9.52 (1H, br s).

### (ii) Production of N-(tert-butyl)-3-(2-chloro-4-{[6-(hydroxymethyl)-8,9-dihydro-7H-pyrimido[4,5-b]azepin-4-yl]amino}phenoxy)benzamide

To a solution of 4-[(4-{3-[(tert-butylamino) carbonyl]phenoxy}-3-chlorophenyl)amino]-8,9-dihydro-7H-pyrimido[4,5-b]azepine-6-carboxylic acid (300 mg) and 4-methylmorpholine (78 μL) in tetrahydrofuran (5.0 mL) was added isopropyl chlorocarbonate (88 μL) at 0° C. The mixture was stirred at 0° C. for 2 hr, and the precipitate was removed by filtration. The filtrate was added dropwise to a mixed solution of sodium borohydride (122 mg) in tetrahydrofuran (3.0 mL) and water (3.0 mL) at 0° C. The mixture was stirred at 0° C. for 1 hr and extracted with ethyl acetate. The organic layer was washed with saturated brine, dried over anhydrous magnesium sulfate and concentrated under reduced pressure. The residue was separated and purified by silica gel column chromatography (eluent, ethyl acetate→methyl acetate:methanol=9:1) to give the title compound (187 mg) as pale-yellow crystals.

¹H-NMR (CDCl₃) δ: 1.45 (9H, s), 2.53 (2H, t, J=4.8 Hz), 3.52 (2H, t, J=4.8 Hz), 4.22 (2H, s), 5.51-5.58 (1H, m), 5.95 (1H, br s), 6.31 (1H, s), 6.41 (1H, s), 6.99 (1H, d, J=8.7 Hz), 7.05-7.10 (1H, m), 7.22-7.28 (2H, m), 7.31-7.38 (2H, m), 7.61 (1H, d, J=2.7 Hz), 8.08 (1H, s).

### Example 168

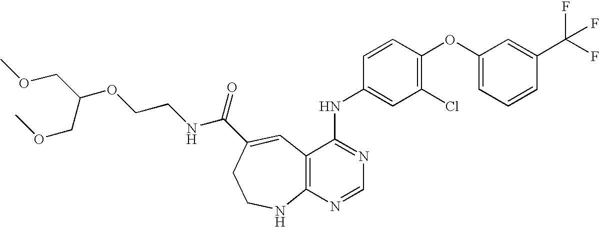

### Production of 4-({3-chloro-4-[3-(trifluoromethyl)phenoxy]phenyl}amino)-N-{2-[2-methoxy-1-(methoxymethyl)ethoxy]ethyl}-8,9-dihydro-7H-pyrimido[4,5-b]azepine-6-carboxamide

### (i) Production of 2-(2-{2-(benzyloxy)-1-[(benzyloxy)methyl]ethoxy}ethoxy)tetrahydro-2H-pyran

A mixture of 2-(2-bromoethoxy)tetrahydro-2H-pyran (3.20 g) and 1,3-bisbenzyloxy-2-propanol (5.00 g) was dissolved in toluene (40 mL). Tetra-n-butylammonium hydrogen sulfate (530 mg) and 50% aqueous sodium hydroxide solution (10 mL) were added to the mixture and the mixture was stirred at 40° C. for 5.5 days. Water was added to the reaction mixture and the mixture was extracted with toluene. The organic layer was washed successively with water and saturated brine, dried over anhydrous magnesium sulfate and concentrated under reduced pressure. The residue was separated and purified by silica gel column chromatography (eluent, hexane:ethyl acetate=98:2→67:33) to give the title, compound (3.16 g) as an orange oil.

¹H-NMR (CDCl₃) δ: 1.38-1.92 (6H, m), 3.40-3.52 (1H, m), 3.52-3.69 (5H, m), 3.69-3.97 (5H, m), 4.54 (4H, s), 4.59-4.71 (1H, m), 7.16-7.46 (10H, m).

### (ii) Production of 2-{2-(benzyloxy)-1-[(benzyloxy)methyl]ethoxy}ethanol

2-(2-{2-(Benzyloxy)-1-[(benzyloxy)methyl]ethoxy}ethoxy)tetrahydro-2H-pyran (3.15 g) was dissolved to methanol (25 mL). 6N hydrochloric acid (4.0 mL) was added to the mixture, and the solution was stirred at 60° C. for 3 days. The reaction mixture was neutralized with 8N aqueous sodium hydroxide solution and concentrated under reduced pressure. Water was added to the residue, and the mixture was extracted with ethyl acetate. The organic layer was washed with saturated brine, dried over anhydrous magnesium sulfate and concentrated under reduced pressure. The residue was separated and purified by silica gel column chromatography (eluent, hexane:ethyl acetate=80:26→33:67) to give the title compound (2.37 g) as a pale-yellow oil.

¹H-NMR (CDCl₃) δ: 3.10 (1H, t, J=3.8 Hz), 3.46-3.63 (4H, m), 3.62-3.90 (5H, m), 4.54 (4H, s), 7.18-7.45 (10H, m).

### (iii) Production of 1,1′-[[2-(2-azidoethoxy)propane-1,3-diyl]bis(oxymethylene)]dibenzene

2-{2-(Benzyloxy)-1-[(benzyloxy)methyl]ethoxy}ethanol (2.37 g) was dissolved in ethyl acetate (30 mL), and the solution was cooled to 0° C. Triethylamine (1.6 mL) and methanesulfonyl chloride (0.65 mL) were added to the mixture and the mixture was stirred at 0° C. for 1 hr. Water was added to the reaction mixture, and the mixture was extracted with ethyl acetate. The organic layer was washed with saturated brine, dried over anhydrous magnesium sulfate and concentrated under reduced pressure. The residue was dissolved in N,N-dimethylformamide (20 mL). Sodium azide (640 mg) was added to the solution, and the mixture was stirred at 50° C. for 15 hr. Additional sodium azide (232 mg) was added to the mixture, and the mixture was stirred at 50° C. for 5 hr. The reaction mixture was concentrated under reduced pressure. Water was added to the residue and the mixture was extracted with ethyl acetate. The organic layer was washed successively with water and saturated brine, dried over anhydrous magnesium sulfate and concentrated under reduced pressure. The residue was separated and purified by silica gel column chromatography (eluent, hexane:ethyl acetate=95:5→67:33) to give the title compound (2.40 g) as a colorless oil.

¹H-NMR (CDCl₃) δ: 3.33-3.41 (2H, m), 3.54-3.66 (4H, m), 3.70-3.79 (1H, m), 3.79-3.86 (2H, m), 4.54 (4H, s), 7.19-7.46 (10H, m).

### (iv) Production of tert-butyl (2-{2-(benzyloxy)-1-[(benzyloxy)methyl]ethoxy}ethyl)carbamate

1,1′-[[2-(2-Azidoethoxy)propane-1,3-diyl]bis(oxymethylene)]dibenzene (2.39 g) was dissolved in a mixture of tetrahydrofuran (23 mL) and water (2 mL). Triphenylphosphine (2.03 g) was added to the mixture, and the mixture was stirred at room temperature for 3 days. The reaction mixture was concentrated under reduced pressure, and the residue was dissolved in methanol (30 mL). The solution was cooled to 0° C. Triethylamine (2.5 mL) and di-tert-butyl dicarbonate (2.5 mL) were added to the solution and the mixture was stirred for 6.5 hr at room temperature. Water was added to the reaction mixture, methanol was evaporated under reduced pressure, and the residue was extracted with ethyl acetate. The organic layer was washed with saturated brine, dried over anhydrous magnesium sulfate and concentrated under reduced pressure. The residue was separated and purified by silica gel column chromatography (eluent, hexane:ethyl acetate=95:5→67:33) to give the title compound (2.76 g) as a colorless oil.

¹H-NMR (CDCl₃) δ: 1.43 (9H, s), 3.20-3.39 (2H, m), 3.45-3.61 (4H, m), 3.61-3.80 (3H, m), 4.54 (4H, s), 5.21-5.46 (1H, m), 7.18-7.45 (10H, m).

### (v) Production of tert-butyl {2-[2-hydroxy-1-(hydroxymethyl)ethoxy]ethyl}carbamate and tert-butyl {2-[2-(benzyloxy)-1-(hydroxymethyl)ethoxy]ethyl}carbamate

tert-Butyl (2-{2-(benzyloxy)-1-[(benzyloxy)methyl]ethoxy}ethyl)carbamate (2.75 g) was dissolved in methanol (30 mL). 10% Palladium-carbon (297 mg) was added to the solution, and the mixture was stirred for 2.5 days under hydrogen atmosphere. The reaction mixture was filtrated through Celite, and the filtrate was concentrated under reduced pressure. The residue was dissolved methanol (30 mL), 10% palladium-carbon (606 mg) was added to the solution, and the mixture was stirred at room temperature for 10 days under hydrogen atmosphere. The reaction mixture was filtrated through Celite, and the filtrate was concentrated under reduced pressure. The residue was separated and purified by silica gel column chromatography (eluent, hexane:ethyl acetate=80:20→0:100) to give tert-butyl {2-[2-hydroxy-1-(hydroxymethyl)ethoxy]ethyl}carbamate (413 mg) and tert-butyl {2-[2-(benzyloxy)-1-(hydroxymethyl)ethoxy]ethyl}carbamate (1.20 g), each as a colorless oil.

### tert-Butyl {2-[2-hydroxy-1-(hydroxymethyl)ethoxy]ethyl}carbamate

¹H-NMR (CDCl₃) δ: 1.45 (9H, s), 2.48 (2H, br s), 3.34 (2H, q, J=5.5 Hz), 3.43-3.57 (1H, m), 3.60-3.87 (6H, m), 5.05 (1H, br s).

### tert-Butyl {2-[2-(benzyloxy)-1-(hydroxymethyl)ethoxy]ethyl}carbamate

¹H-NMR (CDCl₃) δ: 1.44 (9H, s), 2.26 (1H, br s), 3.21-3.42 (2H, m), 3.47-3.81 (7H, m), 4.54 (2H, s), 5.07 (1H, br s), 7.19-7.45 (5H, m).

### (vi) Production of tert-butyl {2-[2-methoxy-1-(methoxymethyl)ethoxy]ethyl}carbamate

60% Sodium hydride (dispersion in mineral oil, 153 mg) was suspended in tetrahydrofuran (15 mL) and the suspension was cooled to 0° C. A solution of tert-butyl {2-[2-hydroxy-1-(hydroxymethyl)ethoxy]ethyl}carbamate (408 mg) in tetrahydrofuran (5.0 mL) was added dropwise to the suspension, and the mixture was stirred at 0° C. for 1 hr. Methyl iodide (0.227 mL) was added to the solution, and the mixture was stirred at room temperature for 3 hr. Additional methyl iodide (0.110 mL) was added to the mixture, and the mixture was stirred for 3 hr. Saturated aqueous ammonium chloride solution was added to the reaction mixture, and the mixture was extracted with ethyl acetate. The organic layer was washed with saturated brine, dried over anhydrous magnesium sulfate and concentrated under reduced pressure. The residue was separated and purified by silica gel column chromatography (eluent, hexane:ethyl acetate=80:20→33:67) to give the title compound (251 mg) as a colorless oil.

¹H-NMR (CDCl₃) δ: 1.44 (9H, m), 3.30 (2H, q, J=5.4 Hz), 3.37 (6H, s), 3.39-3.50 (4H, m), 3.55-3.75 (3H, m), 5.39 (1H, br s).

### (vii) Production of 2-[2-methoxy-1-(methoxymethyl)ethoxy]ethaneamine hydrochloride

tert-Butyl {2-[2-methoxy-1-(methoxymethyl)ethoxy]ethyl}carbamate (246 mg) was dissolved in tetrahydrofuran (3.0 mL). 6N hydrochloric acid (0.5 mL) was added to the solution and the mixture was stirred at 50° C. for 14 hr. The reaction mixture was concentrated under reduced pressure, and the residue was azeotropically evaporated with ethanol. The residue was dried under reduced pressure to give the title compound (185 mg) as a colorless oil.

¹H-NMR (DMSO-d₆) δ: 2.92 (2H, t, J=5.5 Hz), 3.26 (6H, s), 3.37-3.43 (4H, m), 3.57-3.67 (1H, m), 3.71 (2H, t, J=5.5 Hz), 7.98 (3H, br s).

### (viii) Production of 4-({3-chloro-4-[3-(trifluoromethyl)phenoxy]phenyl}amino)-N-{2-[2-methoxy-1-(methoxymethyl)ethoxy]ethyl}-8,9-dihydro-7H-pyrimido[4,5-b]azepine-6-carboxamide

A mixture of 4-({3-chloro-4-[3-(trifluoromethyl)phenoxy]phenyl}amino)-8,9-dihydro-7H-pyrimido[4,5-b]azepine-6-carboxylic acid (119 mg) and 2-[2-methoxy-1-(methoxymethyl)ethoxy]ethaneamine hydrochloride (73.0 mg) was dissolved in a mixture of tetrahydrofuran (0.60 mL) and N,N-dimethylformamide (0.60 mL). Triethylamine (0.35 mL), 1-hydroxybenzotriazole (52.8 mg) and 1-ethyl-3-(3-dimethylaminopropyl)carbodiimide hydrochloride (77.6 mg) were successively added to the mixture, and the mixture was stirred at room temperature for 16 hr. Water was added to the reaction mixture, and the mixture was extracted with ethyl acetate. The organic layer was dried over anhydrous magnesium sulfate and concentrated under reduced pressure. The residue was separated and purified by silica gel column chromatography (eluent, hexane:ethyl acetate=10:90→0:100→ethyl acetate:methanol=90:10). The objective fraction was concentrated under reduced pressure and crystallized from ethyl acetate-diisopropyl ether to give the title compound (32.2 mg) as a white powder.

¹H-NMR (DMSO-d₆) δ: 2.63-2.72 (2H, m), 3.20 (6H, s), 3.25-3.41 (8H, m), 3.52-3.63 (3H, m), 7.12-7.23 (3H, m), 7.26 (1H, d, J=8.7 Hz), 7.46 (1H, d, J=7.6 Hz), 7.55-7.65 (2H, m), 7.70 (1H, t, J=4.7 Hz), 7.88 (1H, d, J=2.7 Hz), 7.95-8.04 (2H, m), 9.17 (1H, s).

### Example 169

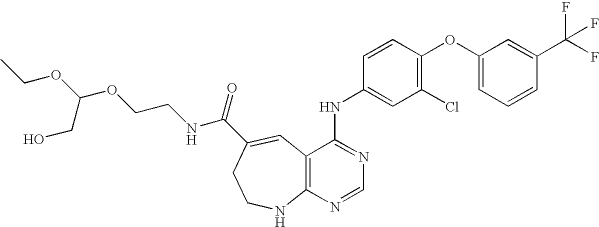

### Production of 4-({4-[3-(trifluoromethyl)phenoxy]-3-chlorophenyl}amino)-N-{2-[2-hydroxy-1-(methoxymethyl)ethoxy]ethyl}-8,9-dihydro-7H-pyrimido[4,5-b]azepine-6-carboxamide

### (i) Production of tert-butyl {2-[2-(benzyloxy)-1-(methoxymethyl)ethoxy]ethyl}carbamate

Using tert-butyl {2-[2-(benzyloxy)-1-(hydroxymethyl)ethoxy]ethyl}carbamate (499 mg), 60% sodium hydride (dispersion in mineral oil, 73.4 mg), methyl iodide (0.11 mL) and tetrahydrofuran (20 mL), a similar reaction as in Example 168 (vi) was carried out to give the title compound (296 mg) as a colorless oil.

¹H-NMR (CDCl₃) δ: 1.44 (9H, s), 3.30 (2H, q, J=5.3 Hz), 3.36 (3H, s), 3.39-3.59 (4H, m), 3.60-3.77 (3H, m), 4.55 (2H, s), 5.37 (1H, br s), 7.18-7.46 (5H, m).

### (ii) Production of tert-butyl {2-[2-hydroxy-1-(methoxymethyl)ethoxy]ethyl}carbamate

Using tert-butyl {2-[2-(benzyloxy)-1-(methoxymethyl)ethoxy]ethyl}carbamate (290 mg), 10% palladium-carbon (47.7 mg) and methanol (3.0 mL), a similar reaction as in Example 168 (v) was carried out to give the title compound (206 mg) as a colorless oil.

¹H-NMR (CDCl₃) δ: 1.45 (9H, s), 2.27 (1H, br s), 3.23-3.41 (2H, m), 3.38 (3H, s), 3.43-3.81 (7H, m), 5.10 (1H, br s).

### (iii) Production of 2-(2-aminoethoxy)-3-methoxypropan-1-ol hydrochloride

Using tert-butyl {2-[2-hydroxy-1-(methoxymethyl)ethoxy]ethyl}carbamate (201 mg), 6N hydrochloric acid (0.50 mL) and tetrahydrofuran (4.0 mL), a similar reaction as in Example 168 (vii) was carried out to give the title compound (150 mg) as a colorless oil.

¹H-NMR (DMSO-d₆) δ: 2.84-3.03 (2H, m), 3.26 (3H, s), 3.29-3.55 (4H, m), 3.57-3.84 (3H, m), 4.80 (1H, br s), 7.94 (3H, br s).

### (iv) Production of 4-({4-[3-(trifluoromethyl)phenoxy]-3-chlorophenyl}amino)-N-{2-[2-hydroxy-1-(methoxymethyl)ethoxy]ethyl}-8,9-dihydro-7H-pyrimido[4,5-b]azepine-6-carboxamide

Using 4-({3-chloro-4-[3-(trifluoromethyl)phenoxy]phenyl}amino)-8,9-dihydro-7H-pyrimido[4,5-b]azepine-6-carboxylic acid (119 mg), 2-(2-aminoethoxy)-3-methoxypropan-1-ol hydrochloride (71.8 mg), tetrahydrofuran (0.60 mL), N,N-dimethylformamide (0.60 mL), triethylamine (0.35 mL), 1-hydroxybenzotriazole (51.5 mg) and 1-ethyl-3-(3-dimethylaminopropyl)carbodiimide hydrochloride (73.3 mg), a similar reaction as in Example 168 (viii) was carried out to give the title compound (94.8 mg) as white crystals.

¹H-NMR (DMSO-d₆) δ: 2.63-2.74 (2H, m), 3.20 (3H, s), 3.25-3.47 (9H, m), 3.58 (2H, t, J=6.1 Hz), 4.64 (1H, t, J=5.3 Hz), 7.12-7.22 (3H, m), 7.26 (1H, d, J=8.7 Hz), 7.46 (1H, d, J=8.0 Hz), 7.54-7.65 (2H, m), 7.69 (1H, t, J=4.5 Hz), 7.89 (1H, d, J=2.3 Hz), 7.93-8.04 (2H, m), 9.15 (1H, s).

### Example 170

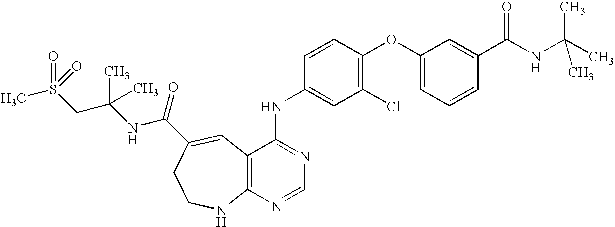

### Production of 4-[(4-{3-[(tert-butylamino)carbonyl]phenoxy}-3-chlorophenyl)amino]-N-[1,1-dimethyl-2-(methylsulfonyl)ethyl]-8,9-dihydro-7H-pyrimido[4,5-b]azepine-6-carboxamide

Using 4-[(4-{3-[(tert-butylamino)carbonyl]phenoxy}-3-chlorophenyl)amino]-8,9-dihydro-7H-pyrimido[4,5-b]azepine-6-carboxylic acid (100 mg), 2-methyl-1-(methylsulfonyl)-2-propylamine hydrochloride (74 mg), 1-ethyl-3-(3-dimethylaminopropyl)carbodiimide hydrochloride (75.6 mg), 1-hydroxybenzotriazole monohydrate (60 mg), triethylamine (82 μL) and N,N-dimethylformamide (5.0 mL), a similar reaction as in Example 136 was carried out to give the title compound (87 mg) as colorless crystals.

¹H-NMR (CDCl₃) δ: 1.46 (9H, s), 1.69 (6H, s), 2.85 (2H, t, J=4.5 Hz), 2.98 (3H, s), 3.47-3.57 (2H, m), 3.63 (2H, s), 5.63-5.71 (1H, m), 5.93 (1H, br s), 6.36 (1H, s), 7.00 (1H, d, J=8.7 Hz), 7.03-7.07 (1H, m), 7.19-7.42 (4H, m), 7.48 (1H, dd, J=8.7, 2.4 Hz), 7.87 (1H, d, J=2.4 Hz), 8.13 (1H, s).

### Example 171

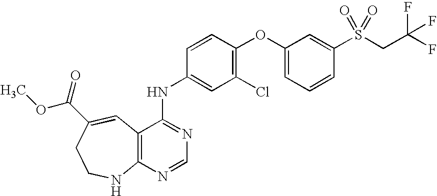

### Production of methyl 4-[(3-chloro-4-{3-[(2,2,2-trifluoroethyl)sulfonyl]phenoxy}phenyl)amino]-8,9-dihydro-7H-pyrimido[4,5-b]azepine-6-carboxylate

Using methyl 4-chloro-8,9-dihydro-7H-pyrimido[4,5-b]azepine-6-carboxylate (100 mg), 3-chloro-4-{3-[(2,2,2-trifluoroethyl)sulfonyl]phenoxy}aniline (153 mg), pyridinium chloride (4.9 mg) and isopropyl alcohol (5.0 mL), a similar reaction as in Example 166 (iv) was carried out to give the title compound (130 mg) as pale-yellow crystals.

¹H-NMR (CDCl₃) δ: 2.92 (2H, t, J=4.8 Hz), 3.56 (2H, q, J=4.8 Hz), 3.83 (3H, s), 3.91 (2H, q, J=9.0 Hz), 5.81-5.89 (1H, m), 6.89 (1H, s), 7.10 (1H, d, J=8.7 Hz), 7.25-7.30 (1H, m), 7.42 (1H, dd, J=8.7, 2.7 Hz), 7.49 (1H, t, J=2.1 Hz), 7.53-7.58 (1H, m), 7.61-7.68 (2H, m), 7.79 (1H, d, J=2.7 Hz), 8.14 (1H, s).

### Example 172

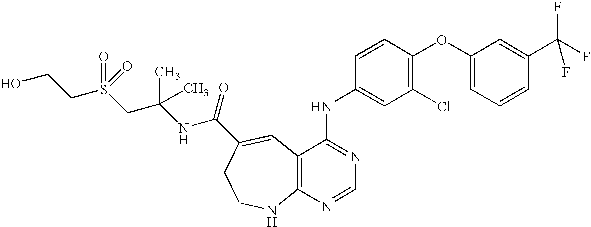

### Production of 4-({3-chloro-4-[3-(trifluoromethyl)phenoxy]phenyl}amino)-N-{2-[(2-hydroxyethyl)sulfonyl]-1,1-dimethylethyl}-8,9-dihydro-7H-pyrimido[4,5-b]azepine-6-carboxamide

### (i) Production of tert-butyl {2-[(2-hydroxyethyl)thio]-1,1-dimethylethyl}carbamate

To a solution of tert-butyl (2-hydroxy-1,1-dimethylethyl)carbamate (10.3 g) and triethylamine (15.2 mL) in tetrahydrofuran (300 mL) was added dropwise methanesulfonyl chloride (6.32 mL) at 0° C. The mixture was stirred at 0° C. for 1 hr, aqueous sodium bicarbonate solution was added to the reaction mixture, and the mixture was extracted with ethyl acetate. The organic layer was washed with saturated brine, dried over anhydrous magnesium sulfate and concentrated under reduced pressure. The residue was separated and purified by silica gel column chromatography (eluent, hexane:ethyl acetate=1:1→1:3) to give a colorless oil (15.2 g). A mixture of the obtained oil (15.2 g), 2-mercaptoethanol (7.63 g) and sodium tert-butoxide (10.5 g) in N,N-dimethylformamide (100 mL) was stirred at 60° C. for 12 hr. Water was added to the reaction mixture, and the mixture was extracted with ethyl acetate. The organic layer was washed with water and saturated brine, dried over anhydrous magnesium sulfate and concentrated under reduced pressure. The residue was separated and purified by silica gel column chromatography (eluent, hexane:ethyl acetate=3:1→3:2) to give the title compound (9.30 g) as a colorless oil.

¹H-NMR (CDCl₃) δ: 1.33 (6H, s), 1.43 (9H, s), 3.01 (3H, s), 4.31 (2H, s), 4.52 (2H, br s).

### (ii) Production of tert-butyl {2-[(2-hydroxyethyl)sulfonyl]-1,1-dimethylethyl}carbamate

To a solution of tert-butyl {2-[(2-hydroxyethyl)thio]-1,1-dimethylethyl}carbamate (9.30 g) in ethyl acetate (300 mL) was added 3-chloroperbenzoic acid (70%, 19.3 g) at 0° C., and the mixture was stirred at 0° C. for 1 hr. Aqueous sodium thiosulfate solution was added to the reaction mixture. The mixture was stirred at room temperature for 1 hr and extracted with ethyl acetate. The organic layer was washed with aqueous sodium bicarbonate solution and saturated brine, dried over anhydrous magnesium sulfate and concentrated under reduced pressure. The residue was separated and purified by basic silica gel column chromatography (eluent, hexane:ethyl acetate=3:1→ethyl acetate) and by silica gel column chromatography (eluent, hexane:ethyl acetate=3:2→1:4) to give the title compound (6.50 g) as a colorless oil.

¹H-NMR (CDCl₃) δ: 1.44 (9H, s), 1.52 (6H, s), 2.94 (1H, t, J=6.3 Hz), 3.18 (2H, t, J=5.3 Hz), 3.67 (2H, s), 4.06-4.13 (2H, m), 4.83 (1H, s).

### (iii) Production of 2-[(2-amino-2-methylpropyl)sulfonyl]ethanol hydrochloride

To a solution of tert-butyl {2-[(2-hydroxyethyl)sulfonyl]-1,1-dimethylethyl}carbamate (6.50 g) in ethanol (130 mL) was added 2N hydrochloric acid (25 mL) at room temperature. The mixture was stirred at 60° C. for 24 hr. 6N hydrochloric acid (3.0 mL) was added to the reaction mixture. The mixture was stirred at 60° C. for 16 hr, and concentrated under reduced pressure to give the title compound (5.20 g) as a colorless oil.

¹H-NMR (DMSO-d₆) δ: 1.47 (6H, s), 3.33 (2H, t, J=5.4 Hz), 3.59 (2H, s), 3.81 (2H, t, J=5.4 Hz), 5.31 (1H, br s), 8.23 (3H, br s).

### (iv) Production of 4-({3-chloro-4-[3-(trifluoromethyl)phenoxy]phenyl}amino)-N-{2-[(2-hydroxyethyl)sulfonyl]-1,1-dimethylethyl}-8,9-dihydro-7H-pyrimido[4,5-b]azepine-6-carboxamide

Using 4-({3-chloro-4-[3-(trifluoromethyl)phenoxy]phenyl}amino)-8,9-dihydro-7H-pyrimido[4,5-b]azepine-6-carboxylic acid (600 mg), 2-[(2-amino-2-methylpropyl)sulfonyl]ethanol hydrochloride (550 mg), 1-ethyl-3-(3-dimethylaminopropyl)carbodiimide hydrochloride (480 mg), 1-hydroxybenzotriazole monohydrate (380 mg), triethylamine (0.53 mL) and N,N-dimethylformamide (12 mL), a similar reaction as in Example 136 was carried out to give the title compound (666 mg) as colorless crystals.

¹H-NMR (CDCl₃) δ: 1.68 (6H, s), 2.71-2.87 (3H, m), 3.22 (2H, t, J=5.0 Hz), 3.52 (2H, q, J=4.6 Hz), 3.75 (2H, s), 4.11 (2H, t, J=4.6 Hz), 5.75 (1H, t, J=4.8 Hz), 6.39 (1H, s), 7.03 (1H, d, J=9.0 Hz), 7.04-7.10 (1H, m), 7.19 (1H, br s), 7.29-7.33 (2H, m), 7.38-7.44 (1H, m), 7.52 (1H, dd, J=9.0, 2.7 Hz), 7.85 (1H, d, J=2.7 Hz), 8.11 (1H, s).

### Example 173

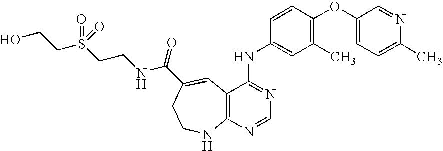

### Production of N-{2-[(2-hydroxyethyl)sulfonyl]ethyl}-4-({3-methyl-4-[(6-methylpyridin-3-yl)oxy]phenyl}amino)-8,9-dihydro-7H-pyrimido[4,5-b]azepine-6-carboxamide

Using 4-({3-methyl-4-[(6-methylpyridin-3-yl)oxy]phenyl}amino)-8,9-dihydro-7H-pyrimido[4,5-b]azepine-6-carboxylic acid (100 mg), 2-[(2-aminoethyl)sulfonyl]ethanol hydrochloride (94 mg), 1-ethyl-3-(3-dimethylaminopropyl)carbodiimide hydrochloride (95 mg), 1-hydroxybenzotriazole monohydrate (76 mg), triethylamine (0.10 mL) and N,N-dimethylformamide (5.0 mL), a similar reaction as in Example 136 was carried out to give the title compound (84 mg) as colorless crystals.

¹H-NMR (DMSO-d₆) δ: 2.14 (3H, s), 2.42 (3H, s), 2.62-2.71 (2H, m), 3.20-3.45 (6H, m), 3.52-3.65 (2H, m), 3.72-3.83 (2H, m), 5.12-5.21 (1H, m), 6.89 (1H, d, J=8.1 Hz), 7.11-7.26 (3H, m), 7.34-7.41 (1H, m), 7.44 (1H, s), 7.55-7.65 (1H, m), 7.90 (1H, s), 8.11-8.18 (1H, m), 8.20-8.28 (1H, m), 8.84 (1H, s).

### Example 174

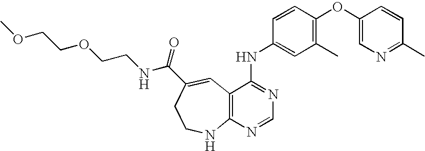

### Production of N-[2-(2-methoxyethoxy)ethyl]-4-({3-methyl-4-[(6-methylpyridin-3-yl)oxy]phenyl}amino)-8,9-dihydro-7H-pyrimido[4,5-b]azepine-6-carboxamide

Using 4-({3-methyl-4-[(6-methylpyridin-3-yl)oxy]phenyl}amino)-8,9-dihydro-7H-pyrimido[4,5-b]azepine-6-carboxylic acid (99.9 mg), 2-(2-methoxyethoxy)ethaneamine hydrochloride (59.3 mg), tetrahydrofuran (0.60 mL), N,N-dimethylformamide (0.60 mL), triethylamine (0.35 mL), 1-hydroxybenzotriazole (51.9 mg) and 1-ethyl-3-(3-dimethylaminopropyl)carbodiimide hydrochloride (71.9 mg), a similar reaction as in Example 168 (viii) was carried out to give the title compound (76.1 mg), as a white powder.

¹H-NMR (DMSO-d₆) δ: 2.14 (3H, s), 2.43 (3H, s), 2.62-2.70 (2H, m), 3.21 (3H, s), 3.28-3.38 (4H, m), 3.39-3.56 (6H, m), 6.90 (1H, d, J=8.9 Hz), 7.11-7.18 (2H, m), 7.19-7.25 (1H, m), 7.37 (1H, dd, J=8.8, 2.5 Hz), 7.44 (1H, d, J=2.5 Hz), 7.55 (1H, t, J=4.5 Hz), 7.90 (1H, s), 8.05 (1H, t, J=5.6 Hz), 8.14 (1H, d, J= 2.5 Hz), 8.86 (1H, s).

### Example 175

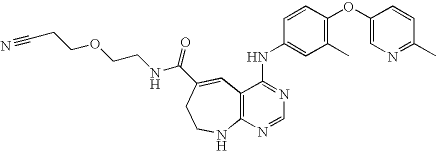

### Production of N-[2-(2-cyanoethoxy)ethyl]-4-({3-methyl-4-[(6-methylpyridin-3-yl)oxy]phenyl}amino)-8,9-dihydro-7H-pyrimido[4,5-b]azepine-6-carboxamide

Using 4-({3-methyl-4-[(6-methylpyridin-3-yl)oxy]phenyl}amino)-8,9-dihydro-7H-pyrimido[4,5-b]azepine-6-carboxylic acid (100 mg), 3-(2-aminoethoxy)propanenitrile hydrochloride (55.7 mg), tetrahydrofuran (0.60 mL), N,N-dimethylformamide (0.60 mL), triethylamine (0.35 mL), 1-hydroxybenzotriazole (52.3 mg) and 1-ethyl-3-(3-dimethylaminopropyl)carbodiimide hydrochloride (71.3 mg), a similar reaction as in Example 168 (viii) was carried out to give the title compound (70.0 mg) as pale-orange crystals.

¹H-NMR (DMSO-d₆) δ: 2.14 (3H, s), 2.43 (3H, s), 2.62-2.71 (2H, m), 2.74 (2H, t, J=6.0 Hz), 3.28-3.40 (4H, m), 3.48-3.57 (2H, m), 3.61 (2H, t, J=6.0 Hz), 6.90 (1H, d, J=8.9 Hz), 7.11-7.19 (2H, m), 7.19-7.25 (1H, m), 7.37 (1H, dd, J=8.7, 2.6 Hz), 7.44 (1H, d, J=2.6 Hz), 7.56 (1H, t, J=4.7 Hz), 7.90 (1H, s), 8.06 (1H, t, J=5.7 Hz), 8.14 (1H, d, J=2.8 Hz), 8.85 (1H, s).

### Example 176

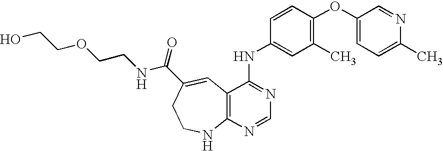

### Production of N-[2-(2-hydroxyethoxy)ethyl]-4-({3-methyl-4-[(6-methylpyridin-3-yl)oxy]phenyl}amino)-8,9-dihydro-7H-pyrimido[4,5-b]azepine-6-carboxamide

Using 4-({3-methyl-4-[(6-methylpyridin-3-yl)oxy]phenyl}amino)-8,9-dihydro-7H-pyrimido[4,5-b]azepine-6-carboxylic acid (100 mg), 2-(2-aminoethoxy)ethanol (52.1 mg), 1-ethyl-3-(3-dimethylaminopropyl)carbodiimide hydrochloride (95 mg), 1-hydroxybenzotriazole monohydrate (76 mg), triethylamine (0.10 mL) and N,N-dimethylformamide (5.0 mL), a similar reaction as in Example 136 was carried out to give the title compound (83 mg) as colorless crystals.

¹H-NMR (CDCl₃) δ: 2.21 (3H, s), 2.51 (3H, s), 2.79-2.94 (3H, m), 3.46-3.66 (8H, m), 3.69-3.79 (2H, m), 5.65-5.71 (1H, m), 6.54-6.65 (1H, m), 6.83 (1H, d, J=8.4 Hz), 7.05-7.14 (2H, m), 7.19-7.31 (4H, m), 8.06 (1H, s), 8.19 (1H, d, J=2.7 Hz).

### Example 177

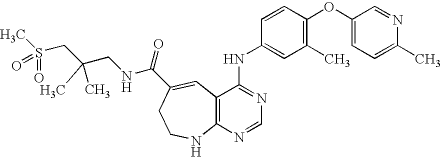

### Production of N-[2,2-dimethyl-3-(methylsulfonyl)propyl]-4-({3-methyl-4-[(6-methylpyridin-3-yl)oxy]phenyl}amino)-8,9-dihydro-7H-pyrimido[4,5-b]azepine-6-carboxamide

Using 4-({3-methyl-4-[(6-methylpyridin-3-yl)oxy]phenyl}amino)-8,9-dihydro-7H-pyrimido[4,5-b]azepine-6-carboxylic acid (100 mg), 2,2-dimethyl-3-(methylsulfonyl)-1-propylamine hydrochloride (100 mg), 1-ethyl-3-(3-dimethylaminopropyl)carbodiimide-hydrochloride (95 mg), 1-hydroxybenzotriazole monohydrate (76 mg), triethylamine (0.10 mL) and N,N-dimethylformamide (5.0 mL), a similar reaction as in Example 136 was carried out to give the title compound (90 mg) as colorless crystals.

¹H-NMR (CDCl₃) δ: 1.23 (6H, s), 2.23 (3H, s), 2.52 (3H, s), 2.86 (3H, s), 2.88 (2H, t, J=4.5 Hz), 3.07 (2H, s), 3.48-3.56 (4H, m), 5.69 (1H, t, J=4.2 Hz), 6.85 (1H, d, J=8.4 Hz), 7.04-7.12 (2H, m), 7.19 (1H, s), 7.23-7.35 (3H, m), 7.40 (1H, d, J=3.0 Hz), 8.08 (1H, s), 8.23 (1H, d, J=2.7 Hz).

### Example 178

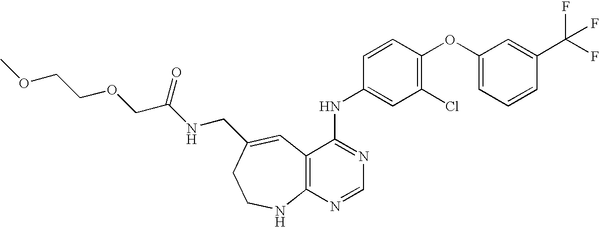

### Production of N-{[4-({3-chloro-4-[3-(trifluoromethyl)phenoxy]phenyl}amino)-8,9-dihydro-7H-pyrimido[4,5-b]azepin-6-yl]methyl}-2-(2-methoxyethoxy)acetamide

Using 6-(aminomethyl)-N-{3-chloro-4-[3-(trifluoromethyl)phenoxy]phenyl}-8,9-dihydro-7H-pyrimido[4,5-b]azepin-4-amine dihydrochloride (50.0 mg), (2-methoxyethoxy)acetic acid (0.016 mL), tetrahydrofuran (0.40 mL), N,N-dimethylformamide (0.40 mL), triethylamine (0.15 mL), 1-hydroxybenzotriazole (26.0 mg) and 1-ethyl-3-(3-dimethylaminopropyl)carbodiimide hydrochloride (36.0 mg), a Similar reaction as in Example 168 (viii) was carried out to give the title compound (32.7 mg) as white crystals.

¹H-NMR (DMSO-d₆) δ: 2.33-2.44 (2H, m), 3.21 (3H, s), 3.26-3.38 (2H, m), 3.38-3.47 (2H, m), 3.54-3.64 (2H, m), 3.87-3.97 (4H, m), 6.42 (1H, s), 7.13-7.21 (2H, m), 7.24 (1H, d, J=8.9 Hz), 7.37 (1H, t, J=4.5 Hz), 7.45 (1H, d, J=7.7 Hz), 7.55-7.66 (2H, m), 7.82 (1H, t, J=5.9 Hz), 7.88 (1H, d, J=2.5 Hz), 7.94 (1H, s), 8.84 (1H, s).

### Example 179

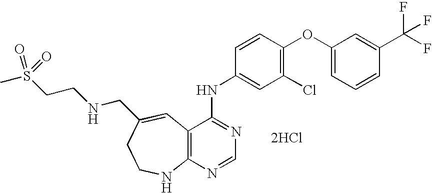

### Production of N-{3-chloro-4-[3-(trifluoromethyl)phenoxy]phenyl}-6-({[2-(methylsulfonyl)ethyl]amino}methyl)-8,9-dihydro-7H-pyrimido[4,5-b]azepin-4-amine dihydrochloride

### (i) Production of N-[2-(methylsulfonyl)ethyl]-2-nitrobenzenesulfonamide

A mixture of 2-nitrobenzenesulfonyl chloride (3.50 g) and 2-(methylsulfonyl)ethaneamine (2.50 g) was dissolved in toluene (30 mL). tetra-n-Butylammonium hydrogen sulfate (560 mg) and 1N aqueous sodium hydroxide solution (10 mL) were added to the solution, and the mixture was stirred at 50° C. for 24 hr. Saturated aqueous ammonium chloride solution was added to the reaction mixture, and the mixture was extracted with ethyl acetate. The organic layer was washed with saturated brine, dried over anhydrous magnesium sulfate and concentrated under reduced pressure. The residue was separated and purified by silica gel column chromatography (eluent, hexane:ethyl acetate=50:50→0:100) to give the title compound (1.37 g) as white crystals.

¹H-NMR (CDCl₃) δ: 3.03 (3H, s), 3.26-3.45 (2H, m), 3.54-3.73 (2H, m), 6.08 (1H, br s), 7.70-7.84 (2H, m), 7.86-7.99° (1H, m), 8.07-8.21 (1H, m).

### (ii) Production of N-{3-chloro-4-[3-(trifluoromethyl)phenoxy]phenyl}-6-({[2-(methylsulfonyl)ethyl]amino}methyl)-8,9-dihydro-7H-pyrimido[4,5-b]azepin-4-amine dihydrochloride

A mixture of [4-({3-chloro-4-[3-(trifluoromethyl)phenoxy]phenyl}amino)-8,9-dihydro-7H-pyrimido[4,5-b]azepin-6-yl]methanol (99.6 mg) and N-[2-(methylsulfonyl)ethyl]-2-nitrobenzenesulfonamide (86.2 mg) was dissolved in tetrahydrofuran (2 mL) and the solution was cooled to 0° C. Triphenylphosphine (84.7 mg) and 40% diethyl azodicarboxylate-toluene solution (0.148 mL) were added to the solution, and the mixture was stirred at 0° C. for 4 hr. The reaction mixture was concentrated under reduced pressure. The residue was separated and purified by silica gel column chromatography (eluent, hexane:ethyl acetate=50:50→0:100) and basic silica gel column chromatography (eluent, hexane:ethyl acetate=50:50→0:100) to give N-{[4-({3-chloro-4-[3-(trifluoromethyl)phenoxy]phenyl}amino)-8,9-dihydro-7H-pyrimido[4,5-b]azepin-6-yl]methyl}-N-[2-(methylsulfonyl)ethyl]-2-nitrobenzenesulfonamide, which was dissolved in tetrahydrofuran (1 mL). Diazabicyclo[5,4,0]-7-undecene (0.0134 mL) and 2-mercaptoethanol (0.0063 mL) were added to the solution, and the mixture was stirred at room temperature for 22 hr. The reaction mixture was concentrated under reduced pressure. The residue was separated and purified by basic silica gel column chromatography (eluent, hexane:ethyl acetate=10:90→0:100→ethyl acetate:methanol=90:10) to give N-{3-chloro-4-[3-(trifluoromethyl)phenoxy]phenyl}-6-({[2-(methylsulfonyl)ethyl]amino}methyl)-8,9-dihydro-7H-pyrimido[4,5-b]azepin-4-amine, which was dissolved in ethyl acetate (1.0 mL). 4N Hydrochloric acid/ethyl acetate (0.030 mL) was added to the solution, and the mixture was stirred at room temperature for 1 hr. The reaction mixture was concentrated under reduced pressure. The precipitate was collected by filtration and dried to give the title compound (17.9 mg) as a pale-orange powder.

¹H-NMR (DMSO-d₆) δ 2.59-2.68 (2H, m), 3.14 (3H, s), 3.26-3.73 (6H, m), 3.76-3.86 (2H, m), 6.91 (1H, s), 7.16-7.26 (2H, m), 7.30 (1H, d, J=8.8 Hz), 7.48 (1H, d, J=8.0 Hz), 7.57-7.68 (2H, m), 7.91 (1H, d, J=2.5 Hz), 8.20 (1H, s), 8.34 (1H, br s), 9.57 (2H, br s), 9.91 (1H, br s).

### Example 180

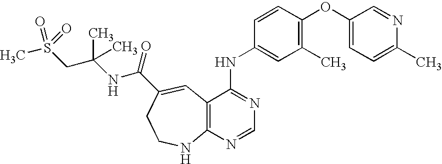

### Production of N-[1,1-dimethyl-2-(methylsulfonyl)ethyl]-4-({3-methyl-4-[(6-methylpyridin-3-yl)oxy]phenyl}amino)-8,9-dihydro-7H-pyrimido[4,5-b]azepine-6-carboxamide

Using 4-({3-methyl-4-[(6-methylpyridin-3-yl)oxy]phenyl}amino)-8,9-dihydro-7H-pyrimido[4,5-b]azepine-6-carboxylic acid (100 mg), 2-methyl-1-(methylsulfonyl)-2-propylamine hydrochloride (93 mg), 1-ethyl-3-(3-dimethylaminopropyl)carbodiimide hydrochloride (95 mg), 1-hydroxybenzotriazole monohydrate (76 mg), triethylamine (0.10 mL) and N,N-dimethylformamide (5.0 mL), a similar reaction as in Example 136 was carried out to give the title compound (86 mg) as colorless crystals.

¹H-NMR (CDCl₃) δ: 1.67 (6H, s), 2.23 (3H, s), 2.52 (3H, s), 2.83 (2H, t, J=4.4 Hz), 2.94 (3H, s), 3.52 (2H, q, J=4.8 Hz), 3.64 (2H, s), 5.65 (1H, t, J=4.8 Hz), 6.26 (1H, s), 6.84 (1H, d, J=8.7 Hz), 7.04-7.11 (3H, m), 7.28 (1H, s), 7.33 (1H, dd, J=8.7, 2.4 Hz), 7.45 (1H, d, J=2.4 Hz), 8.09 (1H, s), 8.24-8.26 (1H, m).

### Example 181

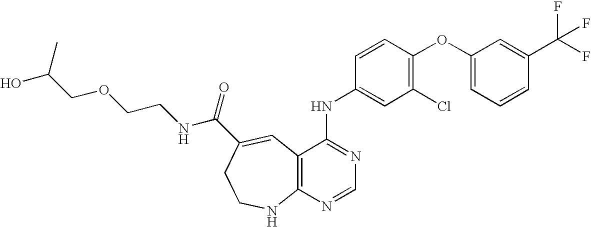

### Production of 4-({3-chloro-4-[3-(trifluoromethyl)phenoxy]phenyl}amino)-N-[2-(2-hydroxypropoxy)ethyl]-8,9-dihydro-7H-pyrimido[4,5-b]azepine-6-carboxamide

### (i) Production of 1-[2-(benzyloxy)propoxy]-4-methoxybenzene

60% Sodium hydride (dispersion in mineral oil, 1.45 g) was suspended in N,N-dimethylformamide (80 mL), and the suspension was cooled to 0° C. A solution of 1-(4-methoxyphenoxy)-2-propanol (5.00 g) in N,N-dimethylformamide (20 mL) was added dropwise to the suspension, and the mixture was stirred at 0° C. for 30 min. Benzyl bromide (4 mL) was added dropwise to the solution, and the mixture was stirred for 2 hr at 0° C. Saturated aqueous ammonium chloride solution was added to the reaction mixture, and the mixture was extracted with diethyl ether. The organic layer was washed successively with water and saturated brine, dried over anhydrous magnesium sulfate and concentrated under reduced pressure. The residue was separated and purified by silica gel column chromatography (eluent, hexane:ethyl acetate=100:0→80:20) to give the title compound (6.58 g) as a colorless oil.

¹H-NMR (CDCl₃) δ: 1.30 (3H, d, J=6.1 Hz), 3.77 (3H, s), 3.80-4.10 (3H, m), 4.67 (2H, s), 6.72-6.92 (4H, m), 7.16-7.46 (5H, m).

### (ii) Production of 2-(benzyloxy)propan-1-ol

1-[2-(Benzyloxy)propoxy]-4-methoxybenzene (6.57 g) was dissolved in a mixture of acetonitrile (160 mL) and water (40 mL). Diammonium cerium nitrate (25.3 g) was added to the solution, and the mixture was stirred at room temperature for 7 hr. Saturated aqueous sodium thiosulfate solution and saturated aqueous sodium hydrogencarbonate solution were added to the reaction mixture, and the mixture was extracted with ethyl acetate. The organic layer was washed with saturated brine, dried over anhydrous magnesium sulfate and concentrated under reduced pressure. The residue was separated and purified by silica gel column chromatography (eluent, hexane:ethyl acetate=95:5→50:50) to give the title compound (3.28 g) as an orange oil.

¹H-NMR (CDCl₃) δ: 1.19 (3H, d, J=6.1 Hz), 2.01 (1H, dd, J=8.0, 4.5 Hz), 3.39-3.80 (3H, m), 4.49 (1H, d, J=11.4 Hz), 4.66 (1H, d, J=11.4 Hz), 7.12-7.49 (5H, m).

### (iii) Production of 2-{2-[2-(benzyloxy)propoxy]ethoxy}tetrahydro-2H-pyran

Using 2-(benzyloxy)propan-1-ol (3.27 g), 2-(2-bromoethoxy)tetrahydro-2H-pyran (4.95 g), toluene (40 mL), tetra-n-butylammonium hydrogen sulfate (670 mg) and 50% aqueous sodium hydroxide solution (10 mL), a similar reaction as in Example 168 (i) was carried out to give the title compound (3.25 g) as a yellow oil.

¹H-NMR (CDCl₃) δ: 1.19 (3H, d, J=6.4 Hz), 1.40-1.93 (6H, m), 3.39-3.98 (9H, m), 4.55-4.72 (3H, m), 7.14-7.45 (5H, m).

### (iv) Production of 2-[2-(benzyloxy)propoxy]ethanol

2-{2-[2-(Benzyloxy)propoxy]ethoxy}tetrahydro-2H-pyran (3.24 g) was dissolved in methanol (35 mL). Pyridinium p-toluenesulfonate (3.96 g) was added to the solution, and the mixture was stirred at room temperature for 5 hr. Saturated aqueous sodium hydrogencarbonate solution was added to the reaction mixture. Methanol was evaporated under reduced pressure, and the residue was extracted with ethyl acetate. The organic layer was washed with saturated brine, dried over anhydrous magnesium sulfate and concentrated under reduced pressure. The residue was separated and purified by silica gel column chromatography (eluent, hexane:ethyl acetate=80:20→33:67) to give the title compound (2.22 g) as a colorless oil.

¹H-NMR (CDCl₃) δ: 1.20 (3H, d, J=6.4 Hz), 2.23 (1H, t, j=6.3 Hz), 3.42-3.65 (4H, m), 3.65-3.84 (3H, m), 4.52-4.70 (2H, m), 7.16-7.47 (5H, m).

### (v) Production of {[2-(2-azidoethoxy)-1-methylethoxy]methyl}benzene

Using 2-[2-(benzyloxy)propoxy]ethanol (2.21 g), methanesulfonyl chloride (0.90 mL), triethylamine (2.2 mL), ethyl acetate (30 mL), sodium azide (894 mg) and N,N-dimethylformamide (20 mL), a similar reaction as in Example 168 (iii) was carried out to give the title compound (2.34 g) as a colorless oil.

¹H-NMR (CDCl₃) δ: 1.21 (3H, d, J=6.3 Hz), 3.33-3.41 (2H, m), 3.43-3.52 (1H, m), 3.52-3.61 (1H, m), 3.63-3.69 (2H, m), 3.69-3.80 (1H, m), 4.56-4.67 (2H, m), 7.19-7.45 (5H, m).

### (vi) Production of tert-butyl {2-[2-(benzyloxy)propoxy]ethyl}carbamate

Using {[2-(2-azidoethoxy)-1-methylethoxy]methyl}benzene (2.33 g), triphenylphosphine (2.86 g), tetrahydrofuran (25 mL), water (2.5 mL), di-tert-butyl dicarbonate (3.0 mL), triethylamine (2.0 mL) and methanol (30 mL), a similar reaction as in Example 168 (iv) was carried out to give the title compound (2.94 g) as a colorless oil.

¹H-NMR (CDCl₃) δ: 1.19 (3H, d, J=6.3 Hz), 1.44 (9H, s), 3.22-3.37 (2H, m), 3.37-3.59 (4H, m), 3.63-3.80 (1H, m), 4.50-4.70 (2H, m), 4.92 (1H, br s), 7.15-7.46 (5H, m).

### (vii) Production of tert-butyl [2-(2-hydroxypropoxy)ethyl]carbamate

Using tert-butyl {2-[2-(benzyloxy)propoxy]ethyl}carbamate (2.93 g), 10% palladium-carbon (330 mg) and methanol (30 mL), a similar reaction as in Example 168 (v) was carried out to give the title compound (2.01 g) as a colorless oil.

¹H-NMR (CDCl₃) δ: 1.15 (3H, d, J=6.4 Hz), 1.45 (9H, s), 2.38 (1H, br s), 3.21-3.38 (3H, m), 3.45 (1H, dd, J=9.4, 3.0 Hz), 3.48-3.63 (2H, m), 3.88-4.06 (1H, m), 4.86 (1H, br s).

### (viii) Production of 1-(2-aminoethoxy)propan-2-ol hydrochloride

tert-Butyl [2-(2-hydroxypropoxy)ethyl]carbamate (1.00 g) was dissolved in ethanol (20 mL). 6N hydrochloric acid (4.0 mL) was added to the solution, and the mixture was stirred at 50° C. for 14 hr. The reaction mixture was concentrated under reduced pressure. Ethanol was added to the residue and the mixture was concentrated again under reduced pressure to give the title compound (701 mg) as a white solid.

¹H-NMR (DMSO-d₆) δ: 1.03 (3H, d, J=6.1 Hz), 2.96 (2H, t, J=5.3 Hz), 3.19-3.34 (2H, m), 3.51-3.68 (2H, m), 3.70-3.85 (1H, m), 4.68 (1H, br s), 7.99 (3H, br s).

### (ix) Production of 4-({3-chloro-4-[3-(trifluoromethyl)phenoxy]phenyl}amino)-N-[2-(2-hydroxypropoxy)ethyl]-8,9-dihydro-7H-pyrimido[4,5-b]azepine-6-carboxamide

Using 4-({3-methyl-4-[(6-methylpyridin-3-yl)oxy]phenyl}amino)-8,9-dihydro-7H-pyrimido[4,5-b]azepine-6-carboxylic acid (109 mg), 1-(2-aminoethoxy)propane-2-ol hydrochloride (63.1 mg), tetrahydrofuran (0.60 mL), N,N-dimethylformamide (0.60 mL), triethylamine (0.30 mL), 1-hydroxybenzotriazole (48.0 mg) and 1-ethyl-3-(3-dimethylaminopropyl)carbodiimide hydrochloride (66.7 mg), a similar reaction as in Example 168 (viii) was carried out to give the title compound (89.1 mg) as a white powder.

¹H-NMR (DMSO-d₆) δ: 0.99 (3H, d, J=6.2 Hz), 2.63-2.72 (2H, m), 3.18-3.29 (2H, m), 3.29-3.39 (4H, m), 3.44-3.52 (2H, m), 3.64-3.79 (1H, m), 4.55 (1H, d, J=4.5 Hz), 7.13-7.23 (3H, m), 7.26 (1H, d, J=8.9 Hz), 7.46 (1H, d, J=7.7 Hz), 7.55-7.65 (2H, m), 7.69 (1H, t, J=4.7 Hz), 7.89 (1H, d, J=2.6 Hz), 7.98 (1H, s), 8.03 (1H, t, J=5.5 Hz), 9.13 (1H, s).

### Example 182

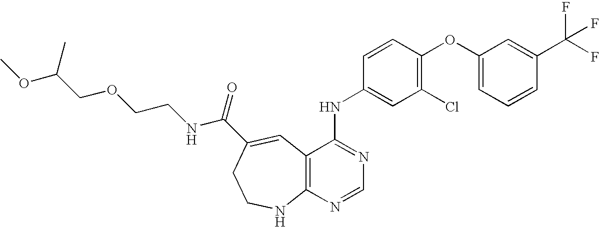

### Production of 4-({3-chloro-4-[3-(trifluoromethyl)phenoxy]phenyl}amino)-N-[2-(2-methoxypropoxy)ethyl]-8,9-dihydro-7H-pyrimido[4,5-b]azepine-6-carboxamide

### (i) Production of tert-butyl [2-(2-methoxypropoxy)ethyl]carbamate

Using tert-butyl [2-(2-hydroxypropoxy)ethyl]carbamate (1.00 g), 60% sodium hydride (dispersion in mineral oil, 222 mg), methyl iodide (0.34 mL) and tetrahydrofuran (60 mL), a similar reaction as in Example 168 (vi) was carried out to give the title compound (552 mg) as a colorless oil.

¹H-NMR (CDCl₃) δ: 1.14 (3H, d, J=6.0 Hz), 1.45 (9H, s), 3.32 (2H, q, J=5.2 Hz), 3.39 (3H, s), 3.41-3.46 (2H, m), 3.46-3.61 (3H, m), 4.98 (1H, br s).

### (ii) Production of 2-(2-methoxypropoxy)ethaneamine hydrochloride

Using tert-butyl 2-(2-methoxypropoxy)ethyl]carbamate (549 mg), 6N hydrochloric acid (2.0 mL) and ethanol (10 mL), a similar reaction as in Example 181 (viii) was carried, out to give the title compound (401 mg) as a pale-yellow oil.

¹H-NMR (DMSO-d₆) δ: 1.07 (3H, d, J=6.1 Hz), 2.95 (2H, t, J=5.3 Hz), 3.26 (3H, s), 3.31-3.52 (3H, m), 3.60 (2H, t, J=5.3 Hz), 7.99 (3H, br s).

### (iii) Production of 4-({3-chloro-4-[3-(trifluoromethyl)phenoxy]phenyl}amino)-N-[2-(2-methoxypropoxy)ethyl]-8,9-dihydro-7H-pyrimido[4,5-b]azepine-6-carboxamide

Using 4-({3-methyl-4-[(6-methylpyridin-3-yl)oxy]phenyl}amino)-8,9-dihydro-7H-pyrimido[4,5-b]azepine-6-carboxylic acid (109 mg), 2-(2-methoxypropoxy)ethaneamine hydrochloride (59.7 mg), tetrahydrofuran (0.60 mL), N,N-dimethylformamide (0.60 mL), triethylamine (0.3 mL), 1-hydroxybenzotriazole (46.8 mg) and 1-ethyl-3-(3-dimethylaminopropyl)carbodiimide hydrochloride (64.6 mg), a similar reaction as in Example 168 (viii) was carried out to give the title compound (102 mg) as a white powder.

¹H-NMR (DMSO-d₆) δ: 1.01 (3H, d, J=6.0 Hz), 2.63-2.72 (2H, m), 3.21 (3H, s), 3.27-3.45 (7H, m), 3.44-3.53 (2H, m), 7.11-7.22 (3H, m), 7.26 (1H, d, J=8.9 Hz), 7.46 (1H, d, J=7.7 Hz), 7.55-7.65 (2H, m), 7.69 (1H, t, J=4.7 Hz), 7.87 (1H, d, J=2.6 Hz), 7.98 (1H, s), 8.03 (1H, t, J=5.7 Hz), 9.14 (1H, s).

### Example 183

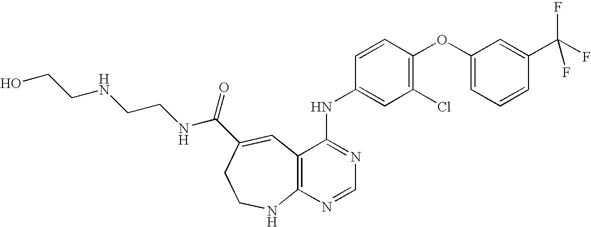

### Production of 4-({3-chloro-4-[3-(trifluoromethyl)phenoxy]phenyl}amino)-N-{2-[(2-hydroxyethyl)amino]ethyl}-8,9-dihydro-7H-pyrimido[4,5-b]azepine-6-carboxamide

### (i) Production of tert-butyl [2-({[4-({3-chloro-4-[3-(trifluoromethyl)phenoxy]phenyl}amino)-8,9-dihydro-7H-pyrimido[4,5-b]azepin-6-yl]carbonyl}amino)ethyl](2-hydroxyethyl)carbamate

Using 4-({3-chloro-4-[3-(trifluoromethyl)phenoxy]phenyl}amino)-8,9-dihydro-7H-pyrimido[4,5-b]azepine-6-carboxylic acid (150 mg), tert-butyl (2-aminoethyl)(2-hydroxyethyl)carbamate (129 mg), 1-ethyl-3-(3-dimethylaminopropyl)carbodiimide hydrochloride (119 mg), 1-hydroxybenzotriazole monohydrate (95 mg), triethylamine (0.13 mL) and N,N-dimethylformamide (5.0 mL), a similar reaction as in Example 136 was carried out to give the title compound (162 mg) as colorless crystals.

¹H-NMR (CDCl₃) δ: 1.33-1.37 (9H, m), 2.62-2.73 (2H, m), 3.12-3.36 (4H, m), 3.41-3.52 (2H, m), 4.66-4.78 (1H, m), 7.13-7.21 (3H, m), 7.25 (1H, d, J=8.7 Hz), 7.45 (1H, d, J=8.1 Hz), 7.56-7.63 (2H, m), 7.66-7.73 (1H, m), 7.85-7.92 (1H, m), 7.97 (1H, s), 8.00-8.09 (1H, m), 9.05-9.14 (1H, m).

### (ii) Production of 4-({3-chloro-4-[3-(trifluoromethyl)phenoxy]phenyl}amino)-N-{2-[(2-hydroxyethyl)amino]ethyl}-8,9-dihydro-7H-pyrimido[4,5-b]azepine-6-carboxamide

To a solution of tert-butyl [2-({[4-({3-chloro-4-[3-(trifluoromethyl)phenoxy]phenyl}amino)-8,9-dihydro-7H-pyrimido[4,5-b]azepin-6-yl]carbonyl}amino)ethyl](2-hydroxyethyl)carbamate (120 mg) in ethanol (5.0 mL) was added 4N hydrochloric acid/ethyl acetate (3.0 mL) at room temperature. The mixture was stirred at room temperature for 24 hr and concentrated under reduced pressure. Aqueous sodium bicarbonate solution was added to the residue, and the mixture was extracted with ethyl acetate. The organic layer was washed with saturated brine, dried over anhydrous magnesium sulfate and concentrated under reduced pressure. The residue was separated and purified by basic silica gel column chromatography (eluent, ethyl acetate->ethyl acetate:methanol=7:3) to give the title compound (84 mg) as colorless crystals.

¹H-NMR (CDCl₃) δ: 2.83-2.90 (6H, m), 3.45-3.57 (4H, m), 3.63-3.70 (2H, m), 5.65-5.71 (1H, m), 6.50-6.60 (1H, m), 7.03 (1H, d, J=9.0 Hz), 7.05-7.11 (1H, m), 7.18-7.21 (1H, m), 7.29-7.33 (2H, m), 7.41-7.47 (2H, m), 7.75 (1H, d, J=2.7 Hz), 7.79 (1H, s), 8.11 (1H, s).

### Example 184

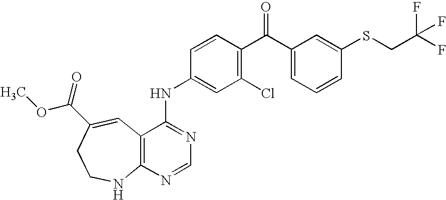

### Production of methyl 4-[(3-chloro-4-{3-[(2,2,2-trifluoroethyl)thio]phenoxy}phenyl)amino]-8,9-dihydro-7H-pyrimido[4,5-b]azepine-6-carboxylate

Using methyl 4-chloro-8,9-dihydro-7H-pyrimido[4,5-b]azepine-6-carboxylate (200 mg), 3-chloro-4-{3-[(2,2,2-trifluoroethyl)thio]phenoxy}aniline (279 mg), pyridinium chloride (9.7 mg) and isopropyl alcohol (2.0 mL), a similar reaction as in Example 166 (iv) was carried out to give the title compound (268 mg) as colorless crystals.

¹H-NMR (CDCl₃) δ: 2.90 (2H, t, J=4.4 Hz), 3.44 (2H, q, J=9.6 Hz), 3.50-3.55 (2H, m), 3.82 (3H, s), 5.79-5.89 (1H, m), 6.82 (1H, s), 6.85-6.88 (1H, m), 7.02 (1H, d, J=9.0 Hz), 7.06-7.08 (1H, m), 7.15-7.19 (1H, m), 7.24-7.30 (1H, m), 7.35 (1H, dd, J=9.0, 2.7 Hz), 7.66 (1H, s), 7.72 (1H, d, J=2.7 Hz), 8.11 (1H, s).

### Example 185

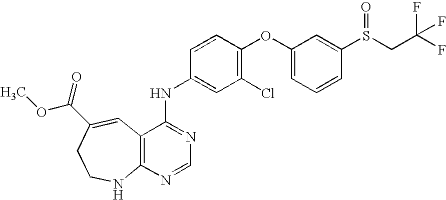

### Production of methyl 4-[(3-chloro-4-{3-[(2,2,2-trifluoroethyl)sulfinyl]phenoxy}phenyl)amino]-8,9-dihydro-7H-pyrimido[4,5-b]azepine-6-carboxylate

To a solution of methyl 4-[(3-chloro-4-{3-[(2,2,2-trifluoroethyl)thio]phenoxy}phenyl)amino]-8,9-dihydro-7H-pyrimido[4,5-b]azepine-6-carboxylate (160 mg) in ethyl acetate (30 mL) was added 3-chloroperbenzoic acid (70%, 110 mg) at −78° C. The mixture was stirred at −78° C. for 1 hr. Additional 3-chloroperbenzoic acid (70%, 80 mg) was added to the reaction mixture, and the mixture was stirred at −40° C. to −10° C. for 2 hr. Aqueous sodium thiosulfate solution was added to the reaction mixture, and the mixture was stirred at room temperature for 12 hr and extracted with ethyl acetate. The organic layer was washed with aqueous sodium bicarbonate solution, saturated brine, dried over anhydrous magnesium sulfate and concentrated under reduced pressure. The residue was separated and purified by silica gel column chromatography (eluent, hexane:ethyl acetate=1:1→methyl acetate) to give the title compound (60 mg) as pale-yellow crystals.

¹H-NMR (CDCl₃) δ: 2.91 (2H, t, J=4.8 Hz), 3.33-3.64 (4H, m), 3.83 (3H, s), 5.76-5.85 (1H, m), 6.83 (1H, s), 7.09 (1H, d, J=8.4 Hz), 7.10-7.14 (1H, m), 7.21-7.29 (1H, m), 7.35-7.42 (2H, m), 7.49-7.54 (1H, m), 7.67 (1H, s), 7.76 (1H, d, J=2.7 Hz), 8.12 (1H, s).

### Example 186

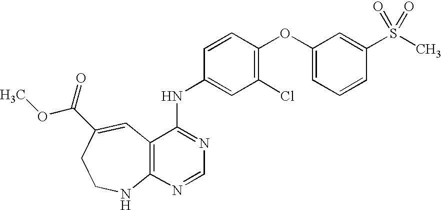

### Production of methyl 4-({3-chloro-4-[3-(methylsulfonyl)-phenoxy]phenyl}amino)-8,9-dihydro-7H-pyrimido[4,5-b]azepine-6-carboxylate

Using methyl 4-chloro-8,9-dihydro-7H-pyrimido[4,5-b]azepine-6-carboxylate (100 mg), 3-chloro-4-[3-(methylsulfonyl)phenoxy]aniline (124 mg), pyridinium chloride (4.8 mg) and isopropyl alcohol (2.0 mL), a similar reaction as in Example 166 (iv) was carried out to give the title compound (117 mg) as pale-yellow crystals.

¹H-NMR (CDCl₃) δ: 2.91 (2H, t, J=4.5 Hz), 3.06 (3H, s), 3.54 (2H, q, J=4.8 Hz), 3.83 (3H, s), 5.78-5.87 (1H, m), 6.83 (1H, s), 7.09 (1H, d, J=8.7 Hz), 7.19-7.23 (1H, m), 7.40 (1H, dd, J=8.7, 2.4 Hz), 7.46-7.54 (2H, m), 7.62-7.67 (2H, m), 7.77 (1H, d, J=2.4 Hz), 8.12 (1H, s).

### Example 187

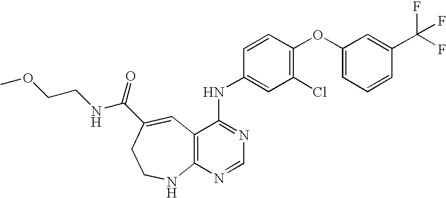

### Production of N-{3-chloro-4-[3-(trifluoromethyl)phenoxy]phenyl}-6-{[(2-methoxyethyl)amino]methyl}-8,9-dihydro-7H-pyrimido[4,5-b]azepin-4-amine dihydrochloride

### (i) Production of N-{[4-({3-chloro-4-[3-(trifluoromethyl)phenoxy]phenyl}amino)-8,9-dihydro-7H-pyrimido[4,5-b]azepin-6-yl]methyl}-N-(2-methoxyethyl)-2-nitrobenzenesulfonamide

A mixture of [4-({3-chloro-4-[3-(trifluoromethyl)phenoxy]phenyl}amino)-8,9-dihydro-7H-pyrimido[4,5-b]azepin-6-yl]methanol (99.7 mg) and N-(2-methoxyethyl)-2-nitrobenzenesulfonamide (72.5 mg) was dissolved in tetrahydrofuran (1.5 mL). Triphenylphosphine (86.2 mg) and diisopropyl azodicarboxylate (0.068 mL) were added to the solution, and the mixture was stirred at 60° C. for 18 hr. The reaction mixture was concentrated under reduced pressure. The residue was separated and purified by silica gel column chromatography (eluent, hexane:ethyl acetate=10:90→0:100→ethyl acetate:methanol=90:10) and basic silica gel column chromatography (eluent, hexane:ethyl acetate=10:90→0:100→ethyl acetate:methanol=90:10) to give the title compound (30.9 mg) as a yellow oil.

¹H-NMR (CDCl₃) δ: 2.48 (2H, t, J=4.7 Hz), 3.19 (3H, s), 3.39-3.58 (6H, m), 4.15 (2H, s), 5.61 (1H, t, J=4.4 Hz), 6.39 (1H, s), 7.00-7.14 (3H, m), 7.20 (1H, br s), 7.28-7.36 (1H, m), 7.37-7.48 (2H, m), 7.63-7.77 (3H, m), 7.84 (1H, d, J=2.7 Hz), 8.08 (2H, m).

### (ii) Production of N-{3-chloro-4-[3-(trifluoromethyl)phenoxy]phenyl}-6-{[(2-methoxyethyl)amino]methyl}-8,9-dihydro-7H-pyrimido[4,5-b]azepin-4-amine dihydrochloride

N-{[4-({3-chloro-4-[3-(trifluoromethyl)phenoxy]phenyl}amino)-8,9-dihydro-7H-pyrimido[4,5-b]azepin-6-yl]methyl}-N-(2-methoxyethyl)-2-nitrobenzenesulfonamide (30.0 mg) was dissolved in tetrahydrofuran (1.0 mL). 2-Mercaptoethanol (0.012 mL) and diazabicyclo[5,4,0]-7-undecene (0.0254 mL) were added to the solution, and the mixture was stirred at room temperature for 24 hr. The reaction mixture was concentrated under reduced pressure. The residue was separated and purified by basic silica gel column chromatography (eluent, hexane:ethyl acetate=20:80→0:100→ethyl acetate:methanol=90:10), and the objective fraction was concentrated under reduced pressure. The residue was dissolved in ethyl acetate (1 mL). 4N Hydrochloric acid/ethyl acetate (0.020 mL) was added to the solution, and the mixture was stirred at room temperature for 1 hr. The reaction mixture was concentrated under reduced pressure and crystallized from ethanol-ethyl acetate to give the title compound (4.8 mg) as yellow crystals.

¹H-NMR (DMSO-d₆) δ: 2.54-2.68 (2H, m), 3.05-3.20 (2H, m), 3.32 (3H, s), 3.36-3.79 (6H, m), 6.55 (1H, s), 6.87 (1H, s), 7.18-7.25 (2H, m), 7.30 (1H, d, J=8.7 Hz), 7.49 (1H, d, J=7.5 Hz), 7.57-7.71 (2H, m), 7.91 (1H, d, J=2.6 Hz), 8.16 (1H, s), 9.14 (2H, br s), 9.74 (1H, br s).

### Example 188

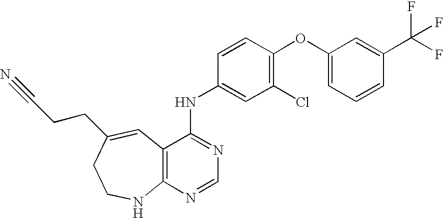

### Production of 3-[4-({3-chloro-4-[3-(trifluoromethyl)phenoxy]phenyl}amino)-8,9-dihydro-7H-pyrimido[4,5-b]azepin-6-yl]propanenitrile

A mixture of [4-({3-chloro-4-[3-(trifluoromethyl)phenoxy]phenyl}amino)-8,9-dihydro-7H-pyrimido[4,5-b]azepin-6-yl]methanol (89.3 mg) and N-[2-(methylsulfonyl)ethyl]-2-nitrobenzenesulfonamide (78.4 mg) was suspended in toluene (1.5 mL). (Tributyl phosphoranylidene)acetonitrile (75.8 mg) was added to the suspension, and the mixture was stirred under reflux for 24 hr. The reaction mixture was concentrated under reduced pressure, and the residue was separated and purified by silica gel column chromatography (eluent, hexane:ethyl acetate=50:50→0:100) and basic silica gel column chromatography (eluent, hexane:ethyl acetate=50:50→0:100) to give the title compound (10.4 mg) and N-{[4-({3-chloro-4-[3-(trifluoromethyl)phenoxy]phenyl}amino)-8,9-dihydro-7H-pyrimido[4,5-b]azepin-6-yl]methyl}-N-[2-(methylsulfonyl)ethyl]-2-nitrobenzenesulfonamide.

¹H-NMR (DMSO-d₆) δ: 2.37-2.58 (4H, m), 2.75 (2H, t, J=7.5 Hz), 3.25-3.39 (2H, m), 6.38 (1H, s), 7.12-7.22 (2H, m), 7.24 (1H, d, J=8.9 Hz), 7.34 (1H, t, J=4.4 Hz), 7.47 (1H, s), 7.54-7.67 (2H, m), 7.89 (1H, d, J=2.5 Hz), 7.93 (1H, s), 8.74 (1H, s).

### Example 189

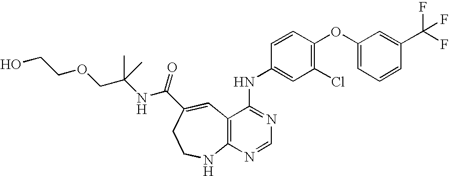

### Production of 4-({3-chloro-4-[3-(trifluoromethyl)phenoxy]phenyl}amino)-N-[2-(2-hydroxyethoxy)-1,1-dimethylethyl]-8,9-dihydro-7H-pyrimido[4,5-b]azepine-6-carboxamide

### (i) Production of tert-butyl {2-[2-(benzyloxy)ethoxy]-1,1-dimethylethyl}carbamate

Using (2-hydroxy-1,1-dimethylethyl) carbamate (1.00 g), benzyl(2-bromoethyl)ether (0.90 mL), tetra-n-butylammonium hydrogen sulfate (183 mg), toluene (10 mL) and 50% aqueous sodium hydroxide solution (2.5 mL), a similar reaction as in Example 168 (i) was carried out to give the title compound (590 mg) as a yellow oil.

¹H-NMR (CDCl₃) δ: 1.31 (6H, s), 1.43 (9H, s), 3.40 (2H, s), 3.56-3.74 (4H, m), 4.58 (2H, s), 4.89 (1H, br s), 7.21-7.44 (5H, m).

### (ii) Production of tert-butyl [2-(2-hydroxyethoxy)-1,1-dimethylethyl]carbamate

Using tert-butyl {2-[2-(benzyloxy)ethoxy]-1,1-dimethylethyl}carbamate (585 mg), 10% palladium-carbon (67.9 mg) and methanol (5.0 mL), a similar reaction as in Example 168 (v) was carried out to give the title compound (412 mg) as a colorless oil.

¹H-NMR (CDCl₃) δ: 1.29 (6H, s), 1.43 (9H, s), 2.10 (1H, t, J=6.1 Hz), 3.48 (2H, s), 3.54-3.66 (2H, m), 3.67-3.83 (2H, m), 4.68 (1H, br s).

### (iii) Production of 2-(2-amino-2-methylpropoxy)ethanol hydrochloride

Using tert-butyl [2-(2-hydroxyethoxy)-1,1-dimethylethyl]carbamate (213 mg), 6N hydrochloric acid (0.80 mL) and ethanol (5.0 mL), a similar reaction as in Example 181 (viii) was carried out to give the title compound (1.60 mg) as a colorless oil.

¹H-NMR (DMSO-d₆) δ: 1.22 (6H, s), 3.39 (2H, s), 3.46-3.59 (4H, m), 8.01 (3H, br s).

### (iv) Production of 4-({3-chloro-4-[3-(trifluoromethyl)phenoxy]phenyl}amino)-N-[2-(2-hydroxyethoxy)-1,1-dimethylethyl]-8,9-dihydro-7H-pyrimido[4,5-b]azepine-6-carboxamide

Using 4-({3-methyl-4-[(6-methylpyridin-3-yl)oxy]phenyl}amino)-8,9-dihydro-7H-pyrimido[4,5-b]azepine-6-carboxylic acid (280 mg), 2-(2-amino-2-methylpropoxy)ethanol hydrochloride (108 mg), tetrahydrofuran (1.4 mL), N,N-dimethylformamide (1.4 mL), triethylamine (0.8 mL), 1-hydroxybenzotriazole (120 mg) and 1-ethyl-3-(3-dimethylaminopropyl)carbodiimide hydrochloride (172 mg), a similar reaction as in Example 168 (viii) was carried out to give the title compound (246 mg) as white crystals.

¹H-NMR (DMSO-d₆) δ: 1.30 (6H, s), 2.59-2.68 (2H, m), 3.28-3.39 (2H, m), 3.40-3.51 (4H, m), 3.53 (2H, s), 4.52-4.60 (1H, m), 7.03 (1H, s), 7.14-7.32 (4H, m), 7.47 (1H, s), 7.53-7.64 (2H, m), 7.66 (1H, t, J=4.5 Hz), 7.94 (1H, d, J=2.6 Hz), 7.99 (1H, s), 9.18 (1H, s).

### Example 190

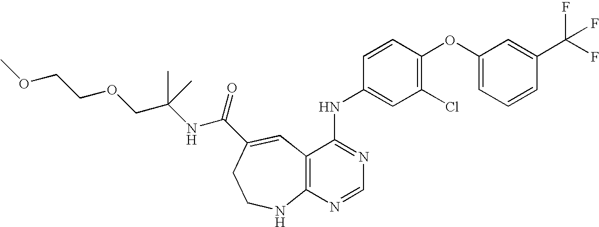

### Production of 4-({3-chloro-4-[3-(trifluoromethyl)phenoxy]phenyl}amino)-N-[2-(2-methoxyethoxy)-1,1-dimethylethyl]-8,9-dihydro-7H-pyrimido[4,5-b]azepine-6-carboxamide

### (i) Production of tert-butyl [2-(2-methoxyethoxy)-1,1-dimethylethyl]carbamate

Using tert-butyl [2-(2-hydroxyethoxy)-1,1-dimethylethyl]carbamate (193 mg), 60% sodium hydride (dispersion in mineral oil, 42.7 mg), methyl iodide (0.0924 mL) and tetrahydrofuran (12 mL), a similar reaction as in Example 168 (vi) was carried out to give the title compound (166 mg) as a colorless oil.

¹H-NMR (CDCl₃) δ: 1.29 (6H, s), 1.43 (9H, s), 3.39 (3H, s), 3.40 (2H, s), 3.51-3.57 (2H, m), 3.60-3.67 (2H, m), 4.91 (1H, br s).

### (ii) Production of 1-(2-methoxyethoxy)-2-methylpropan-2-amine hydrochloride

Using tert-butyl [2-(2-methoxyethoxy)-1,1-dimethylethyl]carbamate (163 mg), 6N hydrochloric acid (0.60 mL) and ethanol (3.5 mL), a similar reaction as in Example 181 (viii) was carried out to give the title compound (123 mg) as a colorless oil.

¹H-NMR (DMSO-d₆) δ: 1.21 (6H, s), 3.27 (3H, s), 3.39 (2H, s), 3.45-3.54 (2H, m), 3.55-3.66 (2H, m), 7.94 (3H, br s).

### (iii) Production of 4-({3-chloro-4-[3-(trifluoromethyl)phenoxy]phenyl}amino)-N-[2-(2-methoxyethoxy)-1,1-dimethylethyl]-8,9-dihydro-7H-pyrimido[4,5-b]azepine-6-carboxamide

Using 4-({3-methyl-4-[(6-methylpyridin-3-yl)oxy]phenyl}amino)-8,9-dihydro-7H-pyrimido[4,5-b]azepine-6-carboxylic acid (281 mg), 1-(2-methoxyethoxy)-2-methylpropane-2-amine hydrochloride (120 mg), tetrahydrofuran (1.4 mL), N,N-dimethylformamide (1.4 mL), triethylamine (0.80 mL), 1-hydroxybenzotriazole (122 mg) and 1-ethyl-3-(3-dimethylaminopropyl)carbodiimide hydrochloride (177 mg), a similar reaction as in Example 168 (viii) was carried out to give the title compound (284 mg) as white crystals.

¹H-NMR (DMSO-d₆) δ: 1.29 (6H, s), 2.58-2.69 (2H, m), 3.18 (3H, s), 3.28-3.38 (2H, m), 3.38-3.44 (2H, m), 3.48-3.57 (4H, m), 7.00 (1H, s); 7.13-7.34 (4H, m), 7.46 (1H, d, J=7.9 Hz), 7.54-7.64 (2H, m), 7.66 (1H, t, J=4.6 Hz), 7.95 (1H, d, J=2.5 Hz), 7.99 (1H, s), 9.20 (1H, s).

### Example 191

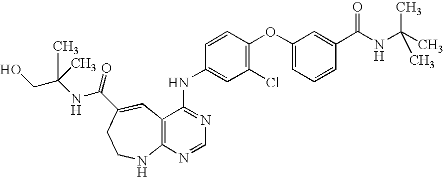

### Production of 4-[(4-{3-[(tert-butylamino) carbonyl]phenoxy}-3-chlorophenyl)amino]-N-(2-hydroxy-1,1-dimethylethyl)-8,9-dihydro-7H-pyrimido[4,5-b]azepine-6-carboxamide

Using 4-[(4-{3-[(tert-butylamino) carbonyl]phenoxy}-3-chlorophenyl)amino]-8,9-dihydro-7H-pyrimido[4,5-b]azepine-6-carboxylic acid (100 mg), 2-amino-2-methyl-1-propanol (35 mg), 1-ethyl-3-(3-dimethylaminopropyl)carbodiimide hydrochloride (75.6 mg), 1-hydroxybenzotriazole monohydrate (60 mg), triethylamine (82 μL) and N,N-dimethylformamide (5.0 mL), a similar reaction as in Example 136 was carried out to give the title compound (91.7 mg) as colorless crystals.

¹H-NMR (DMSO-d₆) δ: 1.26 (6H, s), 1.36 (9H, s), 2.60-2.68 (2H, m), 3.26-3.38 (2H, m), 3.46 (2H, d, J=5.7 Hz), 4.92 (1H, t, J=5.7 Hz), 7.00-7.06 (2H, m), 7.15 (1H, d, J=9.0 Hz), 7.26 (1H, s), 7.30-7.32 (1H, m), 7.37-7.43 (1H, m), 7.51-7.55 (2H, m), 7.63 (1H, t, J=5.1 Hz), 7.80 (1H, s), 7.91 (1H, d, J=2.7 Hz), 7.97 (1H, s), 9.16 (1H, s).

### Example 192

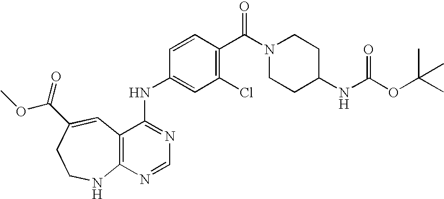

### Production of methyl 4-{[4-({4-[(tert-butoxycarbonyl)amino]piperidin-1-yl}carbonyl)-3-chlorophenyl]amino}-8,9-dihydro-7H-pyrimido[4,5-b]azepine-6-carboxylate

### (i) Production of tert-butyl [1-(2-chloro-4-nitrobenzoyl)piperidin-4-yl]carbamate

2-Chloro-4-nitrobenzoic acid (5.01 g) was dissolved in tetrahydrofuran (50 mL). N,N-Dimethylformamide (0.20 mL) and thionyl chloride (3.6 mL) were added to the solution, and the mixture was stirred at room temperature for 2 hr. The reaction mixture was concentrated under reduced pressure, and the residue was dissolved in tetrahydrofuran (40 mL). The solution was added dropwise to a solution of tert-butyl piperidin-4-ylcarbamate (5.01 g) and triethylamine (10 mL) in tetrahydrofuran (50 mL), and the mixture was stirred at 0° C. for 2 hr. Water was added to the reaction mixture, and the mixture was extracted with ethyl acetate. The organic layer was washed with saturated brine, dried over anhydrous magnesium sulfate and concentrated under reduced pressure. The precipitate was collected by filtration and washed with diisopropyl ether-hexane to give the title compound (9.08 g) as a pale-orange powder.

¹H-NMR (CDCl₃) δ: 1.35-1.61 (11H, m), 1.88-2.19 (2H, m), 2.90-3.44 (3H, m), 3.62-3.82 (1H, m), 4.47 (1H, br s), 4.56-4.78 (1H, m), 7.47 (1H, dd, J=12.2, 8.4 Hz), 8.15-8.23 (1H, m), 8.31 (1H, dd, J=3.5, 2.2 Hz).

### (ii) Production of tert-butyl [1-(4-amino-2-chlorobenzoyl)piperidin-4-yl]carbamate

tert-Butyl [1-(2-chloro-4-nitrobenzoyl)piperidin-4-yl]carbamate (9.04 g) was dissolved in a mixture of ethanol (215 mL) and water (25 mL). Reduced iron (6.64 g) and calcium chloride (1.30 g) were added to the solution, and the mixture was stirred for 21 hr while heating under reflux. The reaction mixture was filtrated through Celite, and the filtrate was concentrated under reduced pressure. The residue was dissolved in ethyl acetate. The solution was washed successively with water and saturated brine, dried over anhydrous magnesium sulfate and concentrated under reduced pressure. The residue was separated and purified by silica gel column chromatography (eluent, hexane:ethyl acetate=80:20→33:67) to give the title compound (7.86 g) as pale-orange amorphous.

¹H-NMR (CDCl₃) δ: 1.33-1.54 (11H, m), 1.81-2.11 (2H, m), 2.82-3.24 (2H, m), 3.40-3.76 (2H, m), 3.85 (2H, s), 4.37-4.54 (1H, m), 4.63 (1H, d, J=12.4 Hz), 6.50-6.61 (1H, m), 6.67 (1H, br s), 6.95-7.11 (1H, m).

### (iii) Production of methyl 4-{[4-({4-[(tert-butoxycarbonyl)amino]piperidin-1-yl}carbonyl)-3-chlorophenyl]amino}-8,9-dihydro-7H-pyrimido[4,5-b]azepine-6-carboxylate

A mixture of methyl 4-chloro-8,9-dihydro-7H-pyrimido[4,5-b]azepine-6-carboxylate (200 mg) and tert-butyl [1-(4-amino-2-chlorobenzoyl)piperidin-4-yl]carbamate (327 mg) was dissolved in 1-methyl-2-pyrrolidone (5 mL). Pyridinium chloride (5.0 mg) was added to the solution, and the mixture was stirred at 80° C. for 17 hr. Pyridinium chloride (50.3 mg) was added to the mixture, the mixture was stirred at 100° C. for 2 days. Saturated aqueous sodium hydrogencarbonate solution was added to the reaction mixture, and the mixture was extracted with ethyl acetate. The organic layer was washed with saturated brine, dried over anhydrous magnesium sulfate and concentrated under reduced pressure. The residue was separated and purified by silica gel column chromatography (eluent, hexane:ethyl acetate=50:50→0:100) and basic silica gel column chromatography (eluent, hexane:ethyl acetate=50:50→0:100) to give the title compound (38.1 mg) as white crystals.

¹H-NMR (DMSO-d₆) δ: 1.38 (9H, s), 1.30-1.44 (2H, m), 1.62-1.89 (2H, m), 2.68-2.78 (2H, m), 2.83-3.17 (2H, m), 3.24-3.43 (3H, m), 3.43-3.60 (1H, m), 3.71 (3H, s), 4.35 (1H, d, J=13.2 Hz), 6.84-6.98 (1H, m), 7.21 (1H, d, J=8.7 Hz), 7.47-7.57 (1H, m), 7.71 (2H, s), 7.94-8.03 (1H, m), 8.00 (1H, s), 9.48 (1H, s).

### Example 193

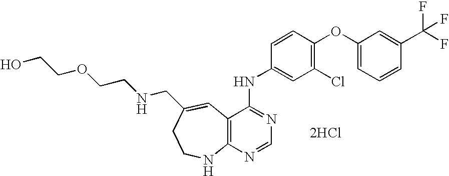

### Production of 2-[2-({[4-({3-chloro-4-[3-(trifluoromethyl)phenoxy]phenyl}amino)-8,9-dihydro-7H-pyrimido[4,5-b]azepin-6-yl]methyl}amino)ethoxy]ethanol dihydrochloride

### (i) Production of 4-({3-chloro-4-[3-(trifluoromethyl)phenoxy]phenyl}amino)-8,9-dihydro-7H-pyrimido[4,5-b]azepin-6-carbaldehyde

[4-({3-Chloro-4-[3-(trifluoromethyl)phenoxy]phenyl}amino)-8,9-dihydro-7H-pyrimido[4,5-b]azepin-6-yl]methanol (110 mg) was dissolved in acetone (2.0 mL). Manganese dioxide (554 mg) was added to the solution, and the mixture was stirred at room temperature for 5.5 hr. The reaction mixture was filtrated through Celite, and the filtrate was concentrated under reduced pressure. The residue was separated and purified by silica gel column chromatography (eluent, hexane:ethyl acetate=50:50→0:100) to give the title compound (80.9 mg) as a yellow solid.

¹H-NMR (CDCl₃) δ: 2.78-2.86 (2H, m), 3.48-3.57 (2H, m), 6.03 (1H, t, J=5.0 Hz), 6.79 (1H, s), 7.07 (1H, d, J=8.9 Hz), 7.12 (1H, dd, J=8.0, 1.8 Hz), 7.17-7.23 (2H, m), 7.31-7.41 (2H, m), 7.44 (1H, t, J=8.0 Hz), 7.74 (1H, d, J=2.6 Hz), 8.17 (1H, s), 9.48 (1H, s).

### (ii) Production of 2-[2-({[4-({3-chloro-4-[3-(trifluoromethyl)phenoxy]phenyl}amino)-8,9-dihydro-7H-pyrimido[4,5-b]azepin-6-yl]methyl}amino)ethoxy]ethanol dihydrochloride

4-({3-Chloro-4-[3-(trifluoromethyl)phenoxy]phenyl}amino)-8,9-dihydro-7H-pyrimido[4,5-b]azepin-6-carbaldehyde (60.5 mg) and 2-aminoethoxyethanol (0.032 mL) were dissolved in tetrahydrofuran (2.0 mL), and the mixture was stirred for 20 min. Sodium triacetoxyborohydride (83.7 mg) was added to the mixture, and the mixture was stirred at room temperature for 1 hr. Sodium triacetoxyborohydride (83.7 mg) was added to the reaction mixture, and the mixture was stirred at room temperature for 1 hr. Additional sodium triacetoxyborohydride (86.6 mg) was added to the mixture, and the mixture was stirred at room temperature for 1 hr. Saturated aqueous ammonium chloride solution was added to the reaction mixture, and the mixture was extracted with ethyl acetate. The organic layer was washed with saturated brine, dried over anhydrous magnesium sulfate and concentrated under reduced pressure. The residue was separated and purified by basic silica gel column chromatography (ethyl acetate:methanol=100:0→90:10), and the objective fraction was concentrated under reduced pressure. The residue was dissolved in ethyl acetate, and 4N hydrochloric acid/ethyl acetate (0.02 mL) was added to the solution. The reaction mixture was concentrated under reduced pressure and crystallized from ethanol-ethyl acetate to give the title compound (9.1 mg) as white crystals.

¹H-NMR (DMSO-d₆) δ: 2.56-2.66 (2H, m), 3.07-3.22 (2H, m), 3.38-3.59 (6H, m), 3.70-3.82 (4H, m), 6.90 (1H, s), 7.17-7.26 (2H, m), 7.31 (1H, d, J=8.9 Hz), 7.49 (1H, d, J=7.5 Hz), 7.57-7.69 (2H, m), 7.91 (1H, d, J=2.5 Hz), 8.19 (1H, s), 8.28 (1H, br s), 9.16 (2H, br s), 9.86 (1H, br s).

### Example 194

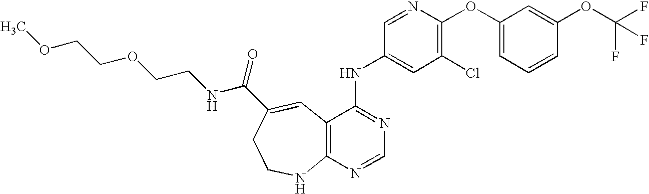

### Production of 4-({5-chloro-6-[3-(trifluoromethoxy)phenoxy]pyridin-3-yl}amino)-N-[2-(2-methoxyethoxy)ethyl]-8,9-dihydro-7H-pyrimido[4,5-b]azepine-6-carboxamide

### (i) Production of 4-({5-chloro-6-[3-(trifluoromethoxy)phenoxy]pyridin-3-yl}amino)-8,9-dihydro-7H-pyrimido[4,5-b]azepine-6-carboxylic acid

To a solution of methyl 4-({5-chloro-6-[3-(trifluoromethoxy)phenoxy]pyridin-3-yl}amino)-8,9-dihydro-7H-pyrimido[4,5-b]azepine-6-carboxylate (235 mg) in tetrahydrofuran (5.0 mL) and ethanol (5.0 mL) was added 1N aqueous sodium hydroxide solution (1.5 mL) at room temperature, and the mixture was stirred at room temperature for 3 days. 1N Hydrochloric acid (1.5 mL) was added to the mixture, and the mixture was concentrated under reduced pressure. The precipitated crystals were collected by filtration and washed with water to give the title compound (195 mg) as pale-yellow crystals.

¹H-NMR (DMSO-d₆) δ: 2.65-2.74 (2H, m), 3.23-3.39 (2H, m), 7.13-7.24 (3H, m), 7.51-7.57 (1H, m), 7.71 (1H, s), 7.83-7.91 (1H, m), 7.94 (1H, s), 8.21 (1H, d, J=2.4 Hz), 8.25 (1H, d, J=2.4 Hz), 9.43 (1H, s).

### (ii) Production of 4-({5-chloro-6-[3-(trifluoromethoxy)phenoxy]pyridin-3-yl}amino)-N-[2-(2-methoxyethoxy)ethyl]-8,9-dihydro-7H-pyrimido[4,5-b]azepine-6-carboxamide

Using 4-({5-chloro-6-[3-(trifluoromethoxy)phenoxy]pyridin-3-yl}amino)-8,9-dihydro-7H-pyrimido[4,5-b]azepine-6-carboxylic acid (80 mg), 2-(2-methoxyethoxy)ethaneamine hydrochloride (50 mg), 1-ethyl-3-(3-dimethylaminopropyl)carbodiimide hydrochloride (62 mg), 1-hydroxybenzotriazole monohydrate (50 mg), triethylamine (70 μL) and N,N-dimethylformamide (5.0 mL), a similar reaction as in Example 136 was carried out to give the title compound (94.4 mg) as colorless crystals.

¹H-NMR (CDCl₃) δ: 2.83 (2H, t, J=4.5 Hz), 3.35 (3H, s), 3.48-3.69 (10H, m), 5.71-5.80 (1H, m), 6.70-6.79 (1H, m), 7.04-7.10 (3H, m), 7.32 (1H, s), 7.37-7.42 (1H, m), 7.73 (1H, br s), 8.07 (1H, s), 8.11 (1H, d, J=2.7 Hz), 8.22 (1H, d, J=2.7 Hz).

### Example 195

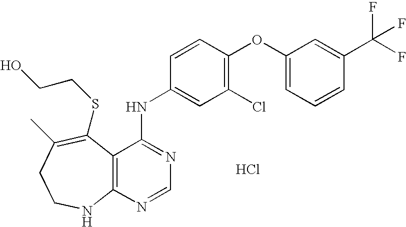

### Production of 2-{[4-({3-chloro-4-[3-(trifluoromethyl)phenoxy]phenyl}amino)-6-methyl-8,9-dihydro-7H-pyrimido[4,5-b]azepin-5-yl]thio}ethanol hydrochloride

### (i) Production of 2-{[tert-butyl (dimethyl)silyl]oxy}ethanethiol

2-Mercaptoethanol (3.0 mL) was dissolved in N,N-dimethylformamide (100 mL). Imidazole (4.39 g) and tert-butyldimethylsilyl chloride (7.09 g) were added to the solution, and the mixture was stirred at room temperature for 2.5 hr. Additional tert-butyldimethylsilyl chloride (1.90 g) was added to the mixture, and the mixture was stirred for 2 hr. Water was added to the reaction mixture, and the mixture was extracted with ethyl acetate. The organic layer was washed successively with water and saturated brine, dried over anhydrous magnesium sulfate and concentrated under reduced pressure. The residue was separated and purified by silica gel column chromatography (eluent, hexane:ethyl acetate=10 0:0→90:10) to give the title compound (7.22 g) as a colorless oil.

¹H-NMR (CDCl₃) δ: 0-08 (6H, s), 0.91 (9H, s), 1.54 (1H, t, J=8.3 Hz), 2.63 (2H, dt, J=8.3, 6.4 Hz), 3.73 (2H, t, J=6.4 Hz).

### (ii) Production of 5-[(2-{[tert-butyl(dimethyl)silyl]oxy}ethyl)thio]-N-{3-chloro-4-[3-(trifluoromethyl)phenoxy]phenyl}-6-methyl-8,9-dihydro-7H-pyrimido[4,5-b]azepin-4-amine

A mixture of 4-({3-chloro-4-[3-(trifluoromethyl)phenoxy]phenyl}amino)-8,9-dihydro-7H-pyrimido[4,5-b]azepin-6-yl]methanol (94.6 mg) and 2-{[tert-butyl(dimethyl)silyl]oxy}ethanethiol (126 mg) was dissolved in tetrahydrofuran (2.0 mL). Triphenylphosphine (68.1 mg) and 40% diethyl azodicarboxylate-toluene solution (0.117 mL) were added to the solution, and the mixture was stirred at room temperature for 2.5 hr. Additional 40% diethyl azodicarboxylate-toluene solution (0.03 mL) was added to the mixture, and the mixture was stirred at 50° C. for 2 days. The reaction mixture was concentrated under reduced pressure, and the residue was separated and purified by silica gel column chromatography (eluent, hexane:ethyl acetate=90:10→33:67) to give the title compound (65.8 mg) as a white solid.

¹H-NMR (CDCl₃) δ: 0.03 (3H, s), 0.04 (3H, s), 0.86 (9H, s), 2.58-2.86 (4H, m), 3.27-3.43 (1H, m), 3.70-3.87 (1H, m), 3.90-4.03 (1H, m), 4.04-4.17 (1H, m), 4.78 (1H, s), 5.04 (1H, s), 5.11 (1H, s), 5.15-5.22 (1H, m), 7.07 (1H, d, J=8.7 Hz), 7.08-7.13 (1H, m), 7.14-7.17 (1H, m), 7.31 (1H, d, J=7.9 Hz), 7.42 (1H, t, J=7.9 Hz), 7.47 (1H, dd, J=8.8, 2.5 Hz), 7.74 (1H, d, J=2.5 Hz), 7.98 (1H, br s), 8.14 (1H, s).

### (iii) Production of 2-{[4-({3-chloro-4-[3-(trifluoromethyl)phenoxy]phenyl}amino)-6-methyl-8,9-dihydro-7H-pyrimido[4,5-b]azepin-5-yl]thio}ethanol hydrochloride

5-[(2-{[tert-Butyl(dimethyl)silyl]oxy}ethyl)thio]-N-{3-chloro-4-[3-(trifluoromethyl)phenoxy]phenyl}-6-methyl-8,9-dihydro-7H-pyrimido[4,5-b]azepin-4-amine (64.0 mg) was dissolved in tetrahydrofuran (4 mL). 1N Tetra-n-butylammonium fluoride-tetrahydrofuran solution (0.110 mL) was added to the solution, and the mixture was stirred at room temperature for 2 hr. Saturated aqueous ammonium chloride solution was added to the reaction mixture, and the mixture was extracted with ethyl acetate. The organic layer was washed with saturated brine, dried over anhydrous magnesium sulfate and concentrated under reduced pressure. The residue was separated and purified by silica gel column chromatography (eluent, hexane:ethyl acetate=80:20→33:67), and the objective fraction was concentrated under reduced pressure. The residue was dissolved in ethyl acetate (1.0 mL). 4N Hydrochloric acid/ethyl acetate (0.05 mL) was added to the solution, and the mixture was stirred at room temperature for 1 hr. The reaction mixture was concentrated under reduced pressure and crystallized from ethyl acetate to give the title compound (39.5 mg) as white crystals.

¹H-NMR (DMSO-d₆) δ: 2.58-2.82 (4H, m), 3.33-3.50 (1H, m), 3.59-3.82 (3H, m), 3.93 (1H, dt, J=11.0, 4.5 Hz), 4.78 (1H, s), 5.07 (1H, s), 5.14 (1H, s), 7.18-7.28 (2H, m), 7.32 (1H, d, J=8.7 Hz), 7.50 (1H, d, J=7.2 Hz), 7.54-7.70 (2H, m), 7.86 (1H, br s), 7.93 (1H, d, J=2.7 Hz), 8.23 (1H, s), 9.30 (1H, br s).

### Example 196

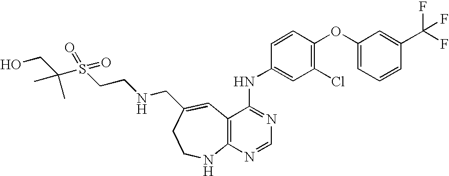

### Production of 2-{[2-({[4-({3-chloro-4-[3-(trifluoromethyl)phenoxy]phenyl}amino)-8,9-dihydro-7H-pyrimido[4,5-b]azepin-6-yl]methyl}amino)ethyl]sulfonyl}-2-methylpropan-1-ol

### (i) Production of tert-butyl 2-({2-[(tert-butoxycarbonyl)amino]ethyl}thio)-2-methylpropanoate

A mixture of tert-butyl 2-bromoisobutyrate (2.0 g), tert-butyl (2-mercaptoethyl)carbamate (1.51 mL) and potassium carbonate (1.23 g) in N,N-dimethylformamide (40 mL) was stirred at 70° C. for 2 days. Water was added to the reaction mixture, and the mixture was extracted with diethyl ether. The organic layer was washed with water and saturated brine, dried over anhydrous magnesium sulfate and concentrated under reduced pressure. The residue was separated and purified by column chromatography (eluent, hexane:ethyl acetate=9:1→1:1) to give the title compound (2.22 g) as a colorless oil.

¹H-NMR (CDCl₃) δ: 1.44 (9H, s), 1.47 (15H, s), 2.78 (2H, t, J=6.5 Hz), 3.23-3.34 (2H, m), 4.83-4.95 (1H, m).

### (ii) Production of tert-butyl {2-[(2-hydroxy-1,1-dimethylethyl)thio]ethyl}carbamate

To a solution of tert-butyl 2-({2-[(tert-butoxycarbonyl)amino]ethyl}thio)-2-methylpropanoate (2.22 g) in toluene (20 mL) was added dropwise 1.5 M diisobutylaluminum hydride-toluene solution (20 mL) at 0° C. The mixture was stirred at 0° C. for 2 hr. Sodium sulfate decahydrate (15.5 g) was added to the reaction mixture, and the mixture was stirred at room temperature for 12 hr. Anhydrous magnesium sulfate was added to the mixture, and the insoluble material was removed by filtration. The filtrate was concentrated, and the residue was separated and purified by column chromatography (eluent, hexane:ethyl acetate=3:1→3:7) to give the title compound (1.19 g) as a colorless oil.

¹H-NMR (CDCl₃) δ: 1.28 (6H, s), 1.45 (9H, s), 2.30-2.41 (1H, m), 2.61 (2H, t, J=6.6 Hz), 3.29 (2H, q, J=6.6 Hz), 3.38 (2H, d, J=6.3 Hz), 4.79-4.93 (1H, m).

### (iii) Production of tert-butyl {2-[(2-hydroxy-1,1-dimethylethyl)sulfonyl]ethyl}carbamate

Using tert-butyl {2-[(2-hydroxy-1,1-dimethylethyl)thio]ethyl}carbamate (1.19 g), 3-chloroperbenzoic acid (70%, 2.59 g) and ethyl acetate (20 mL), a similar reaction as in Example 172 (ii) was carried out to give the title compound (1.30 g) as a colorless oil.

¹H-NMR (CDCl₃) δ: 1.40 (6H, s), 1.44 (9H, s), 2.48 (1H, t, J=5.7 Hz), 3.27 (2H, t, J=5.9 Hz), 3.72 (2H, q, J=5.9 Hz), 3.83 (2H, d, J=5.7 Hz), 5.18-5.30 (1H, m).

### (iv) Production of 2-[(2-aminoethyl)sulfonyl]-2-methylpropan-1-ol hydrochloride

To a solution of tert-butyl {2-[(2-hydroxy-1,1-dimethylethyl)sulfonyl]ethyl}carbamate (1.30 g) in tetrahydrofuran (20 mL) was added 6N hydrochloric acid (5.0 mL) at room temperature. The mixture was stirred at 60° C. for 14 hr, and concentrated under reduced pressure. Ethanol was added to the residue, and the mixture was concentrated again. Crystallization from ethanol-diisopropyl ether gave the title compound (938 mg) as colorless crystals.

¹H-NMR (DMSO-d₆) δ: 1.25 (6H, s), 3.20 (2H, t, J=7.5 Hz), 3.54 (2H, t, J=7.5 Hz), 3.59 (2H, d, J=3.6 Hz), 5.58-5.70 (1H, m), 8.14 (3H, m).

### (v) Production of 2-{[2-({[4-({3-chloro-4-[3-(trifluoromethyl)phenoxy]phenyl}amino)-8,9-dihydro-7H-pyrimido[4,5-b]azepin-6-yl]methyl}amino)ethyl]sulfonyl}-2-methylpropan-1-ol

A mixture of 4-({3-chloro-4-[3-(trifluoromethyl)phenoxy]phenyl}amino)-8,9-dihydro-7H-pyrimido[4,5-b]azepin-6-carbaldehyde (100 mg) and 2-[(2-aminoethyl)sulfonyl]-2-methylpropan-1-ol hydrochloride (93.0 mg) was dissolved in a mixture of tetrahydrofuran (1.5 mL) and N,N-dimethylformamide (1.5 mL). Sodium triacetoxyborohydride (284 mg) was added to the solution, and the mixture was stirred at room temperature for 13 hr. Saturated aqueous sodium hydrogencarbonate solution was added to the reaction solution, and the mixture was extracted with ethyl acetate. The organic layer was dried over anhydrous magnesium sulfate and concentrated under reduced, pressure. The residue was separated and purified by basic silica gel column chromatography (eluent, hexane:ethyl acetate=10:90→0:100→ethyl acetate:methanol=90:10) and crystallized from ethyl acetate-diisopropyl ether to give the title compound (78.7 mg) as white crystals.

¹H-NMR (DMSO-d₆) δ: 1.22 (6H, s), 2.21 (1H, br s), 2.41-2.47 (2H, m), 2.94 (2H, t, J=7.1 Hz), 3.25-3.31 (6H, m), 3.57 (2H, d, J=3.8 Hz), 5.36-5.47 (1H, m), 6.43 (1H, s), 7.14-7.21 (2H, m), 7.23 (1H, d, J=8.9 Hz), 7.32 (1H, t, J=4.3 Hz), 7.45 (1H, d, J=7.7 Hz), 1.55-7.65 (2H, m), 7.87 (1H, d, J=2.6 Hz), 7.92 (1H, s), 8.69 (1H, s).

### Example 197

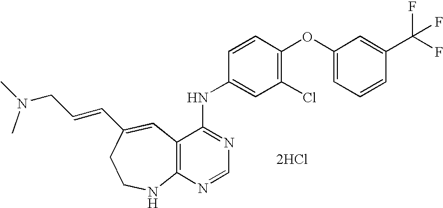

### Production of N-{3-chloro-4-[3-(trifluoromethyl)phenoxy]phenyl}-6-[(1E)-3-(dimethylamino)propan-1-ene-1-yl]-8,9-dihydro-7H-pyrimido[4,5-b]azepin-4-amine dihydrochloride

N,N-Dimethylaminoethyltriphenylphosphonium bromide (357 mg) was suspended in tetrahydrofuran (3.0 mL), and the suspension was cooled to −78° C. n-Butyllithium-hexane solution (1.6 M, 0.6 mL) was added to the suspension, and the mixture was stirred for 1 hr while rising the temperature to room temperature. A solution of 4-({3-chloro-4-[3-(trifluoromethyl)phenoxy]phenyl}amino)-8,9-dihydro-7H-pyrimido[4,5-b]azepin-6-carbaldehyde (96.4 mg) in tetrahydrofuran (1.0 mL) was dropwise added to the solution at −78° C., the mixture was stirred at 50° C. for 3 days. Saturated aqueous ammonium chloride solution was added to the reaction mixture, and the mixture was extracted with ethyl acetate. The organic layer was washed with, saturated brine, dried over anhydrous magnesium sulfate and concentrated under reduced pressure. The residue was separated and purified by basic silica gel column chromatography (eluent, hexane:ethyl acetate=20:80→0:100→ethyl acetate:methanol=95:5), and the objective fraction was concentrated under reduced pressure. The residue was treated with 4N hydrochloric acid/ethyl acetate and crystallized from ethanol-ethyl acetate to give the title compound (6.4 mg) as orange crystals.

¹H-NMR (DMSO-d₆) δ: 2.61-2.69 (2H, m), 2.72 (3H, s), 2.74 (3H, s), 3.42-3.53 (2H, m), 3.74-3.84 (2H, m), 5.73 (1H, dt, J=15.2, 7.6 Hz), 6.63 (1H, d, J=15.2 Hz), 6.76 (1H, s), 7.19-7.26 (2H, m), 7.30 (1H, d, J=8.7 Hz), 7.49 (1H, d, J=8.0 Hz), 7.55 (1H, dd, J=8.7, 2.5 Hz), 7.59-7.68 (1H, m), 7.84 (1H, d, J=2.5 Hz), 8.15 (1H, s), 8.34 (1H, br s), 9.79 (1H, br s), 10.30 (1H, br s).

### Example 198

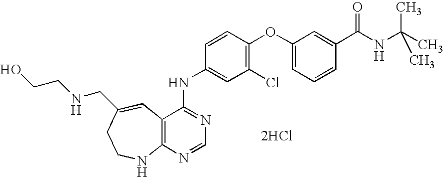

### Production of N-(tert-butyl)-3-{2-chloro-4-[(6-{[(2-hydroxyethyl)amino]methyl}-8,9-dihydro-7H-pyrimido[4,5-b]azepin-4-yl)amino]phenoxy}benzamide dihydrochloride

### (i) Production of N-(tert-butyl)-3-{2-chloro-4-[(6-formyl-8,9-dihydro-7H-pyrimido[4,5-b]azepin-4-yl)amino]phenoxy}benzamide

A mixture of N-(tert-butyl)-3-(2-chloro-4-{[6-(hydroxymethyl)-8,9-dihydro-7H-pyrimido[4,5-b]azepin-4-yl]amino}phenoxy)benzamide (250 mg) and active manganese dioxide (1.25 g) in acetone (12.5 mL) was stirred at room temperature for 24 hr. Manganese dioxide was removed by filtration, and the filtrate was concentrated. The residue was separated and purified by silica gel column chromatography (eluent, hexane:ethyl acetate=1:1→ethyl acetate) to give the title compound (154 mg) as yellow crystals.

¹H-NMR (CDCl₃) δ: 1.46 (9H, s), 2.80 (2H, t, J=4.5 Hz), 3.48-3.53 (2H, m), 5.93 (1H, br s), 5.97-6.05 (1H, m), 6.92 (1H, br s), 7.00 (1H, d, J=8.7 Hz), 7.05-7.09 (1H, m), 7.25 (1H, s), 7.27-7.39 (4H, m), 7.68 (1H, d, J=2.7 Hz), 8.14 (1H, s), 9.46 (1H, s).

### (ii) Production of N-(tert-butyl)-3-{2-chloro-4-[(6-{[(2-hydroxyethyl)amino]methyl}-8,9-dihydro-7H-pyrimido[4,5-b]azepin-4-yl)amino]phenoxy}benzamide dihydrochloride

To a solution of N-(tert-butyl)-3-{2-chloro-4-[(6-formyl-8,9-dihydro-7H-pyrimido[4,5-b]azepin-4-yl)amino]phenoxy}benzamide (140 mg) and 2-aminoethanol (35 mg) in tetrahydrofuran (10 mL) was added sodium triacetoxyborohydride (120 mg) at room temperature. The mixture was stirred at room temperature for 20 hr. Sodium triacetoxyborohydride (120 mg) was added to the reaction mixture, and the mixture was stirred for 16 hr. Aqueous sodium bicarbonate solution was added to the reaction mixture, and the mixture was extracted with ethyl acetate. The organic layer was washed with water and saturated brine, dried over anhydrous magnesium sulfate and concentrated under reduced pressure. The residue was separated and purified by basic silica gel column chromatography (eluent, ethyl acetate→ethyl acetate:methanol=4:1) to give pale-yellow amorphous. 4N Hydrochloric acid/ethyl acetate (0.5 mL) was added to a solution of the obtained amorphous in ethanol (5.0 mL) at room temperature. The mixture was concentrated under reduced pressure and crystallized from ethanol-ethyl acetate to give the title compound (96.7 mg) as pale-brown crystals.

¹H-NMR (DMSO-d₆) δ: 1.36 (9H, s), 2.55-2.67 (2H, m), 2.96-3.07 (2H, m), 3.39-3.49 (2H, m), 3.68-3.81 (4H, m), 6.89 (1H, s), 7.10 (1H, dd, J=7.8, 2.4 Hz), 7.18 (1H, d, J=9.0 Hz), 7.28-7.34 (1H, m), 7.41-7.46 (1H, m), 7.54-7.59 (2H, m), 7.80-7.88 (2H, m), 8.19 (1H, s), 8.26-8.46 (1H, m), 9.05-9.18 (2H, m), 9.84-10.00 (1H, m).

### Example 199

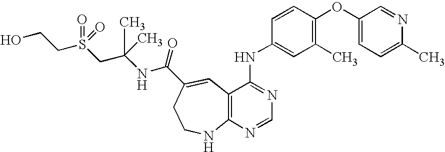

### Production of N-{2-[(2-hydroxyethyl)sulfonyl]-1,1-dimethylethyl}-4-({3-methyl-4-[(6-methylpyridin-3-yl)oxy]phenyl}amino)-8,9-dihydro-7H-pyrimido[4,5-b]azepine-6-carboxamide

Using 4-({3-methyl-4-[(6-methylpyridin-3-yl)oxy]phenyl}amino)-8,9-dihydro-7H-pyrimido[4,5-b]azepine-6-carboxylic acid (120 mg), 2-[(2-amino-2-methylpropyl)sulfonyl]ethanol hydrochloride (130 mg), 1-ethyl-3-(3-dimethylaminopropyl)carbodiimide hydrochloride (114 mg), 1-hydroxybenzotriazole monohydrate (91 mg), triethylamine (0.10 mL) and N,N-dimethylformamide (5.0 mL), a similar reaction as in Example 136 was carried out to give the title compound (70 mg) as colorless crystals.

¹H-NMR (CDCl₃) δ: 1.66 (6H, s), 2.23 (3H, s), 2.51 (3H, s), 2.83 (2H, t, J=4.7 Hz), 2.93-3.03 (1H, m), 3.18 (2H, t, J=5.0 Hz), 3.66-3.75 (2H, m), 3.74 (2H, s), 4.03-4.11 (2H, m), 5.64-5.70 (1H, m), 6.29 (1H, s), 6.84 (1H, d, J=8.7 Hz), 7.04-7.13 (2H, m), 7.15 (1H, s), 7.23-7.32 (2H, m), 7.42-7.43 (1H, m), 8.08 (1H, s), 8.20-8.22 (1H, m).

### Example 200

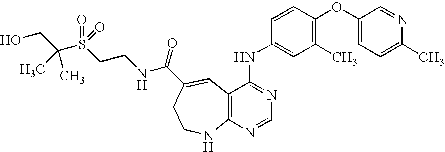

### Production of N-{2-[(2-hydroxy-1,1-dimethylethyl)sulfonyl]ethyl}-4-({3-methyl-4-[(6-methylpyridin-3-yl)oxy]phenyl}amino)-8,9-dihydro-7H-pyrimido[4,5-b]azepine-6-carboxamide

Using 4-({3-methyl-4-[(6-methylpyridin-3-yl)oxy]phenyl}amino)-8,9-dihydro-7H-pyrimido[4,5-b]azepine-6-carboxylic acid (100 mg), 2-[(2-aminoethyl)sulfonyl]-2-methyl-1-propanol hydrochloride (108 mg), 1-ethyl-3-(3-dimethylaminopropyl)carbodiimide hydrochloride (95 mg), 1-hydroxybenzotriazole monohydrate (76 mg), triethylamine (0.10 mL) and N,N-dimethylformamide (5.0 mL), a similar reaction as in Example 136 was carried out to give the title compound (70 mg) as colorless crystals.

¹H-NMR (DMSO-d₆) δ: 1.23 (6H, s), 2.14 (3H, s), 2.43 (3H, s), 2.63-2.69 (2H, m), 3.25-3.39 (4H, m), 3.53-3.66 (4H, m), 5.44 (1H, t, J=5.0 Hz), 6.90 (1H, d, J=8.4 Hz), 7.13-7.24 (3H, m), 7.37-7.42 (1H, m), 7.43-7.46 (1H, m), 7.58 (1H, t, J=4.7 Hz), 7.91 (1H, s), 8.14 (1H, d, J= 3.0 Hz), 8.21 (1H, t, J=6.0 Hz), 8.84 (1H, s).

### Example 201

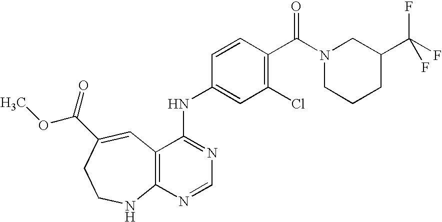

### Production of methyl 4-[(4-{[3-(trifluoromethyl)piperidin-1-yl]carbonyl}-3-chlorophenyl)amino]-8,9-dihydro-7H-pyrimido[4,5-b]azepine-6-carboxylate

### (i) Production of 1-(2-chloro-4-nitrobenzoyl)-3-(trifluoromethyl)piperidine

Using 2-chloro-4-nitrobenzoic acid (5.01 g), tetrahydrofuran (20 mL), N,N-dimethylformamide (0.1 mL), thionyl chloride (0.85 mL), 3-(trifluoromethyl)piperidine (1.10 g) and triethylamine (1.8 mL), a similar reaction as in Example 192 (i) was carried out to give the title compound (1.90 g) as a pale-yellow oil.

¹H-NMR (CDCl₃) δ: 1.31-1.86 (2H, m), 1.88-2.24 (2H, m), 2.28-2.53 (1H, m), 2.69-3.22 (2H, m), 3.26-3.54 (1H, m), 4.49-5.02 (1H, m), 7.42-7.53 (1H, m), 8.16-8.25 (1H, m), 8.29-8.35 (1H, m).

(ii) Production of 3-chloro-4-{[3-(trifluoromethyl)piperidin-1-yl]carbonyl}aniline

Using 1-(2-chloro-4-nitrobenzoyl)-3-(trifluoromethyl)piperidine (1.88 g), ethanol (54 mL), water (6 mL), reduced iron (1.59 g) and calcium chloride (338 mg), a similar reaction as in Example 192

### (ii) was carried out to give the title compound (1.08 g) as white crystals

¹H-NMR (CDCl₃) δ: 1.25-1.98 (3H, m), 1.97-2.20 (1H, m), 2.21-2.56 (1H, m), 2.60-3.16 (2H, m), 3.43-3.76 (1H, m), 3.87 (2H, br s), 4.54-5.07 (1H, m), 6.47-6.62 (1H, m), 6.68 (1H, br s), 6.93-7.16 (1H, m).

### (iii) Production of methyl 4-[(4-{[3-(trifluoromethyl)piperidin-1-yl]carbonyl}-3-chlorophenyl)amino]-8,9-dihydro-7H-pyrimido[4,5-b]azepine-6-carboxylate

A mixture of methyl 4-chloro-8,9-dihydro-7H-pyrimido[4,5-b]azepine-6-carboxylate (150 mg) and 3-chloro-4-{[3-(trifluoromethyl)piperidin-1-yl]carbonyl}aniline (210 mg) was dissolved in a mixture of diisopropyl ether (2.0 mL) and 1-methyl-2-pyrrolidone (2.0 mL). Pyridinium chloride (8.1 mg) was added to the solution, and the mixture was stirred at 100° C. for 16 hr. Saturated aqueous sodium hydrogencarbonate solution was added to the reaction mixture, and the mixture was extracted with ethyl acetate. The organic layer was washed with saturated brine, dried over anhydrous magnesium sulfate and concentrated under reduced pressure. The residue was separated and purified by silica gel column chromatography (eluent, hexane:ethyl acetate=50:50→0:100) and basic silica gel column chromatography (eluent, hexane:ethyl acetate=67:33→10:90) and crystallized from ethyl acetate-diisopropyl ether to give the title compound (182 mg) as a white powder.

¹H-NMR (DMSO-d₅) δ: 1.33-2.08 (4H, m), 2.38-2.66 (1H, m), 2.67-2.79 (2H, m), 2.84-3.23 (2H, m), 3.23-3.53 (3H, m), 3.71 (3H, s), 4.19-4.67 (1H, m), 7.16-7.43 (1H, m), 7.49-7.60 (1H, m), 7.68-7.78 (2H, m), 7.94-8.05 (2H, m), 9.49 (1H, s).

### Example 202

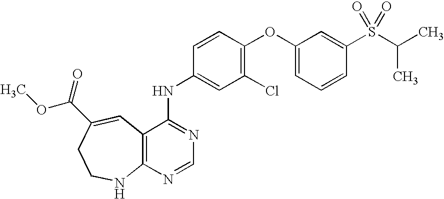

### Production of methyl 4-({3-chloro-4-[3-(isopropylsulfonyl)phenoxy]phenyl}amino)-8,9-dihydro-7H-pyrimido[4,5-b]azepine-6-carboxylate

A solution of methyl 4-chloro-8,9-dihydro-7H-pyrimido[4,5-b]azepine-6-carboxylate (200 mg), 3-chloro-4-[3-(isopropylsulfonyl)phenoxy]aniline (272 mg) and pyridinium chloride (9.6 mg) in 1-methyl-2-pyrrolidone (5.0 mL) was stirred at 100° C. for 2 days. Aqueous sodium bicarbonate solution was added to the reaction mixture, and the mixture was extracted with ethyl acetate. The organic layer was washed with water and saturated brine, dried over anhydrous magnesium sulfate and concentrated under reduced pressure. The residue was separated and purified by silica gel column chromatography (eluent, hexane:ethyl acetate=1:1→ethyl acetate) to give the title compound (277 mg) as pale-yellow crystals.

¹H-NMR (CDCl₃) δ: 1.30 (6H, d, J=6.9 Hz), 2.91 (2H, t, J=4.7 Hz), 3.13-3.25 (1H, m), 3.51-3.56 (2H, m), 3.83 (3H, s), 5.74-5.84 (1H, m), 6.86 (1H, s), 7.07 (1H, d, J=8.4 Hz), 7.18-7.27 (1H, m), 7.37-7.41 (2H, m), 7.47-7.53 (1H, m), 7.55-7.61 (1H, m), 7.67 (1H, s), 7.77 (1H, d, J=2.4 Hz), 8.12 (1H, s).

### Example 203

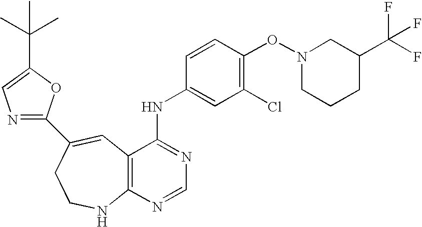

### Production of 6-(5-tert-butyl-1,3-oxazol-2-yl)-N-{3-chloro-4-[3-(trifluoromethyl)phenoxy]phenyl}-8,9-dihydro-7H-pyrimido[4,5-b]azepin-4-amine

### (i) Production of 4-({3-chloro-4-[3-(trifluoromethyl)phenoxy]phenyl}amino)-N-(3,3-dimethyl-2-oxobutyl)-8,9-dihydro-7H-pyrimido[4,5-b]azepine-6-carboxamide

Using 4-({3-methyl-4-[(6-methylpyridin-3-yl)oxy]phenyl}amino)-8,9-dihydro-7H-pyrimido[4,5-b]azepine-6-carboxylic acid (251 mg), 1-amino-3,3-dimethylbutan-2-one (104 mg), tetrahydrofuran (1.3 mL), N,N-dimethylformamide (1.3 mL), triethylamine (0.2 mL), 1-hydroxybenzotriazole (109 mg) and 1-ethyl-3-(3-dimethylaminopropyl)carbodiimide hydrochloride (150 mg), a similar reaction as in Example 168 (viii) was carried out to give the title compound (160 mg) as a pale-yellow powder.

¹H-NMR (CDCl₃) δ: 1.23 (9H, s), 2.83-2.91 (2H, m), 3.53-3.62 (2H, m), 4.38 (2H, d, J=4.1 Hz), 5.73 (1H, t, J=4.7 Hz), 6.69 (1H, t, J=4.1 Hz), 7.04 (1H, d, J=9.0 Hz), 7.10 (1H, dd, J=8.0, 2.3 Hz), 7.15 (1H, s), 7.22 (1H, br s), 7.33 (1H, d, J=8.0 Hz), 7.37-7.46 (2H, m), 7.50 (1H, s), 7.77 (1H, d, J=2.7 Hz), 8.13 (1H, s).

### (ii) Production of 6-(5-tert-butyl-1,3-oxazol-2-yl)-N-{3-chloro-4-[3-(trifluoromethyl)phenoxy]phenyl}-8,9-dihydro-7H-pyrimido[4,5-b]azepin-4-amine

4-({3-Chloro-4-[3-(trifluoromethyl)phenoxy]phenyl}amino)-N-(3,3-dimethyl-2-oxobutyl)-8,9-dihydro-7H-pyrimido[4,5-b]azepine-6-carboxamide (154 mg) was dissolved in pyridine (8.0 mL). Phosphoryl chloride (2.0 mL) was added dropwise to the solution, and the mixture was stirred at room temperature for 3 hr. The reaction mixture was poured into saturated aqueous sodium hydrogencarbonate solution at 0° C., and the mixture was extracted with ethyl acetate. The organic layer was dried over anhydrous magnesium sulfate. The residue was separated and purified by silica gel column chromatography (eluent, hexane:ethyl acetate=33:67→0:100) and crystallized from ethyl acetate-diisopropyl ether to give the title compound (29.7 mg) as a yellow powder.

¹H-NMR (CDCl₃) δ: 1.32 (9H, s), 3.05-3.13 (2H, m), 3.57-3.67 (2H, m), 6.03 (1H, t, J=4.2 Hz), 6.73 (1H, s), 7.04 (1H, d, J=8.9 Hz), 7.08 (1H, dd, J=8.4, 2.2 Hz), 7.15 (1H, s), 7.21 (1H, br s), 7.29-7.46 (4H, m), 7.75 (1H, d, J=2.5 Hz), 8.12 (1H, s).

### Example 204

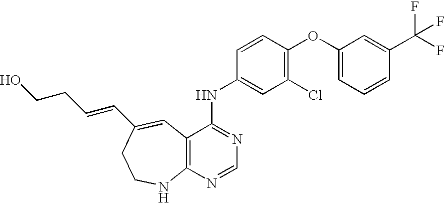

### Production of (3E)-4-[4-({3-chloro-4-[3-(trifluoromethyl)phenoxy]phenyl}amino)-8,9-dihydro-7H-pyrimido[4,5-b]azepin-6-yl]but-3-en-1-ol

### (i) Production of (3-{[tert-butyl(dimethyl)silyl]oxy}propyl)(triphenyl)phosphonium bromide

(3-Bromopropoxy)-tert-butyldimethylsilane (3.00 g) was dissolved in acetonitrile (15 mL). Triphenylphosphine (3.26 g) was added to the solution, and the mixture was stirred for 4 days while heating under reflux. The reaction mixture was concentrated under reduced pressure. The residue was dissolved in methanol, and extracted with hexane to remove excess triphenylphosphine. The methanol layer was concentrated under reduced pressure, and the precipitate was collected by filtration to give the title compound (5.89 g) as colorless crystals.

¹H-NMR (CDCl₃) δ: 0.04 (6H, s), 0.86 (9H, s), 1.81-2.01 (2H, m), 3.79-4.03 (4H, m), 7.53-8.02 (15H, m).

### (ii) Production of 6-[(1E)-4-{[tert-butyl(dimethyl)silyl]oxy}but-1-en-1-yl]-N-{3-chloro-4-[3-(trifluoromethyl)phenoxy]phenyl}-8,9-dihydro-7H-pyrimido[4,5b]azepin-4-amine

(3-{[tert-Butyl(dimethyl)silyl]oxy}propyl)(triphenyl)phosphonium bromide (558 mg) was suspended in tetrahydrofuran (2.0 mL), and the suspension was cooled to 0° C. n-Butyllithium-hexane solution (1.6 M, 0.75 mL) was added dropwise to the suspension, and the mixture was stirred at room temperature for 30 min. A solution of 4-({3-chloro-4-[3-(trifluoromethyl)phenoxy]phenyl}amino)-8,9-dihydro-7H-pyrimido[4,5-b]azepine-6-carbaldehyde (100 mg) in tetrahydrofuran (1.0 mL) was added dropwise to the solution at 0° C., the mixture was stirred at 50° C. for 16 hr. Saturated aqueous ammonium chloride solution was added to the reaction mixture, and the mixture was extracted with ethyl acetate. The organic layer was dried over anhydrous magnesium sulfate and concentrated under reduced pressure. The residue was separated and purified by silica gel column chromatography (eluent, hexane:ethyl acetate=80:20→33:67) and crystallized from diisopropyl ether-hexane to give the title compound (70.6 mg) as a white powder.

¹H-NMR (CDCl₃) δ: 0.07 (6H, s), 0.91 (9H, s), 2.34-2.46 (2H, m), 2.64-2.74 (2H, m), 3.49-3.59 (2H, m), 3.70 (2H, t, J=6.7 Hz), 5.61-5.82 (2H, m), 6.17 (1H, s), 6.27 (1H, d, J=15.7 Hz), 6.58 (1H, s), 7.05 (1H, d, J=8.7 Hz), 7.09 (1H, dd, J=8.3, 2.1 Hz), 7.19 (1H, br s), 7.28-7.49 (3H, m), 7.74 (1H, d, J=2.6 Hz), 8.10 (1H, s).

### (iii) Production of (3E)-4-[4-({3-chloro-4-[3-(trifluoromethyl)phenoxy]phenyl}amino)-8,9-dihydro-7H-pyrimido[4,5-b]azepin-6-yl]but-3-en-1-ol

6-[(1E)-4-{[tert-Butyl(dimethyl)silyl]oxy}but-1-en-1-yl]-N-{3-chloro-4-[3-(trifluoromethyl)phenoxy]phenyl}-8,9-dihydro-7H-pyrimido[4,5-b]azepin-4-amine (70.0 mg) was dissolved in tetrahydrofuran (1.0 mL). IN tetra-n-butylammonium fluoride-tetrahydrofuran solution (0.20 mL) was added to the solution, and the mixture was stirred at room temperature for 6 hr. Saturated aqueous ammonium chloride solution was added to the reaction mixture, and the mixture was extracted with ethyl acetate. The organic layer was dried over anhydrous magnesium sulfate and concentrated under reduced pressure. The residue was separated and purified by silica gel column chromatography (eluent, hexane:ethyl acetate=50:50→0:100), and crystallized from ethyl acetate-diisopropyl ether to give the title compound (14.0 mg) as a pale-yellow powder.

¹H-NMR (DMSO-d₆) δ: 2.24-2.35 (2H, m), 2.52-2.59 (2H, m), 3.28-3.39 (2H, m), 3.42-3.52 (2H, m), 4.55 (1H, t, J=5.4 Hz), 5.59-5.74 (1H, m), 6.31 (1H, d, J=15.8 Hz), 6.49 (1H, s), 7.15-7.21 (2H, m), 7.24 (1H, d, J=8.9 Hz), 7.41-7.49 (2H, m), 7.56-7.67 (2H, m), 7.92 (1H, d, J=2.6 Hz), 7.93 (1H, s), 8.92 (1H, s).

### Example 205

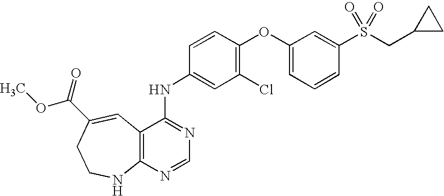

### Production of methyl 4-[(3-chloro-4-{3-[(cyclopropylmethyl)sulfonyl]phenoxy}phenyl)amino]-8,9-dihydro-7H-pyrimido[4,5-b]azepine-6-carboxylate

Using methyl 4-chloro-8,9-dihydro-7H-pyrimido[4,5-b]azepine-6-carboxylate (200 mg), 3-chloro-4-{3-[(cyclopropylmethyl)sulfonyl]phenoxy}aniline (310 mg), pyridinium chloride (9.7 mg) and 1-methyl-2-pyrrolidone (4.0 mL), a similar reaction as in Example 202 was carried out to give the title compound (373 mg) as pale-yellow crystals.

¹H-NMR (CDCl₃) δ: 0.12-0.18 (2H, m), 0.54-0.61 (2H, m), 0.92-1.06 (1H, m), 2.91 (2H, t, J=4.8 Hz), 3.01 (2H, d, J=6.9 Hz), 3.53 (2H, q, J=4.8 Hz), 3.83 (3H, s), 5.79-5.89 (1H, m), 6.88 (1H, s), 7.08 (1H, d, J=8.7 Hz), 7.20-7.28 (1H, m), 7.39 (1H, dd, J=8.7, 2.7 Hz), 7.44-7.46 (1H, m), 7.47-7.53 (1H, m), 7.62-7.64 (1H, m), 7.67 (1H, s), 7.77 (1H, d, J=2.7 Hz), 8.12 (1H, s).

### Example 206

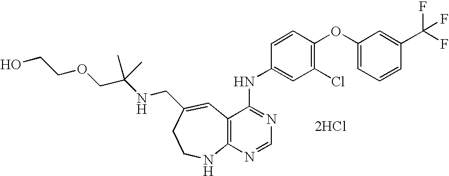

### Production of 2-[2-({[4-({3-chloro-4-[3-(trifluoromethyl)phenoxy]phenyl}amino)-8,9-dihydro-7H-pyrimido[4,5-b]azepin-6-yl]methyl}amino)-2-methylpropoxy]ethanol dihydrochloride

Using 4-({3-chloro-4-[3-(trifluoromethyl)phenoxy]phenyl}amino)-8,9-dihydro-7H-pyrimido[4,5-b]azepine-6-carbaldehyde (95.0 mg), 2-(2-amino-2-methylpropoxy)ethanol hydrochloride (40.1 mg), sodium triacetoxyborohydride (264 mg), tetrahydrofuran (1.5 mL) and N,N-dimethylformamide (1.5 mL), a similar reaction as in Example 193 (ii) was carried out to give the title compound (15.8 mg) as a yellow powder.

¹H-NMR (DMSO-d₆) δ: 1.37 (6H, s), 2.57-2.67 (2H, m), 3.29-3.62 (8H, m), 3.64-3.78 (2H, m), 6.89 (1H, s), 7.17-7.26 (2H, m), 7.30 (1H, d, J=8.9 Hz), 7.48 (1H, d, J=−7.9 Hz), 7.62 (1H, t, J=7.9 Hz), 7.70 (1H, dd, J=8.9, 2.2 Hz), 7.98 (1H, d, J=2.2 Hz), 8.11 (1H, br s), 8.16 (1H, s), 8.90 (2H, br s), 9.82 (1H, br s).

### Example 207

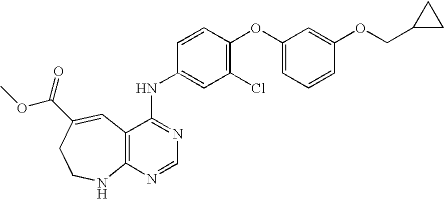

### Production of methyl 4-({3-chloro-4-[3-(cyclopropylmethoxy)phenoxy]phenyl}amino)-8,9-dihydro-7H-pyrimido[4,5-b]azepine-6-carboxylate

A mixture of methyl 4-chloro-8,9-dihydride-7H-pyrimido[4,5-b]azepine-6-carboxylate (102 mg), 3-chloro-4-[3-(cyclopropylmethoxy)phenoxy]aniline (147 mg), pyridinium chloride (8.2 mg) in 1-methyl-2-pyrrolidone (4 mL) and 2-propanol (2 mL) was stirred at 90° C. for 60 hr. Aqueous sodium bicarbonate solution was added to the reaction mixture, and the mixture was extracted with ethyl acetate. The organic layer was washed with saturated brine, dried over anhydrous magnesium sulfate and concentrated under reduced pressure. The residue was separated and purified by silica gel column chromatography (eluent, hexane:ethyl acetate=100:0→25:75) to give the title compound (137 mg) as colorless crystals.

¹H-NMR (DMSO-d₆) δ: 0.27-0.34 (2H, m), 0.50-0.61 (2H, m), 1.11-1.27 (1H, m), 2.70-2.77 (2H, m), 2.89 (1H, s), 3.33-3.42 (1H, m), 3.72 (3H, s), 3.79 (2H, d, J=6.8 Hz), 6.42 (1H, dd, J=7.6, 1.9 Hz), 6.46 (1H, t, J=2.3 Hz), 6.65 (1H, dd, J=7.6, 2.3 Hz), 7.12 (1H, d, J=8.7 Hz), 7.22 (1H, t, J=8.1 Hz), 7.50 (1H, dd, J=8.9, 2.5 Hz), 7.73 (1H, s), 7.79 (1H, d, J=2.3 Hz), 7.92 (1H, t, J=4.9 Hz), 7.98 (1H, s), 9.37 (1H, s).

### Example 208

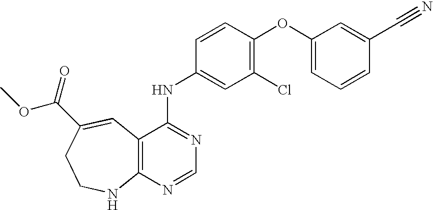

### Production of methyl 4-({3-chloro-4-(3-cyanophenoxy)phenyl}amino)-8,9-dihydro-7H-pyrimido[4,5-b]azepine-6-carboxylate

A mixture of methyl 4-chloro-8,9-dihydride-7H-pyrimido[4,5-b]azepine-6-carboxylic acid (101 mg), 3-(4-amino-2-chlorophenoxy)benzonitrile (113 mg), pyridinium chloride (12.4 mg) in 1-methyl-2-pyrrolidone (5 mL) was stirred at 90° C. for 17 hr. Aqueous sodium bicarbonate solution was added to the reaction mixture, and the mixture was extracted with ethyl acetate. The organic layer was washed with saturated brine, dried over anhydrous magnesium sulfate and concentrated under reduced pressure. The residue was separated and purified by silica gel column chromatography (eluent, hexane:ethyl acetate=100:0→0:100) to give the title compound (96.9 mg) as colorless crystals.

¹H-NMR (DMSO-d₆) δ: 2.70-2.78 (2H, m), 3.37 (2H, q, J=4.8 Hz), 3.72 (3H, s), 7.19-7.28 (2H, m), 7.42 (1H, d, J=3.0 Hz), 7.57 (3H, d, J=4.9 Hz), 7.73 (1H, s), 7.84 (1H, d, J=2.6 Hz), 7.95 (1H, t, J=5.1 Hz), 7.99 (1H, s), 9.44 (1H, s).

### Example 209

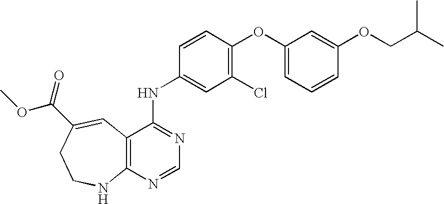

### Production of methyl 4-{[3-chloro-4-(3-isobutoxyphenoxy)phenyl]amino}-8,9-dihydro-7H-pyrimido[4,5-b]azepine-6-carboxylate

A mixture of methyl 4-chloro-8,9-dihydride-7H-pyrimido[4,5-b]azepine-6-carboxylate (102 mg), 3-chloro-4-(3-isobutoxyphenoxy)aniline (141 mg), pyridinium chloride (10.3 mg), 1-methyl-2-pyrrolidone (4 mL) and 2-propanol (2 mL) was stirred at 90° C. for 16 hr. Aqueous sodium bicarbonate solution was added to the reaction mixture, and the mixture was extracted with ethyl acetate. The organic layer was washed with saturated brine; dried over anhydrous magnesium sulfate and concentrated under reduced pressure. The residue was separated and purified by silica gel column chromatography (eluent, hexane:ethyl acetate=100:0→25:75) to give the title compound (150 mg) as colorless crystals.

¹H-NMR (DMSO-d₆) δ: 0.96 (6H, d, J=6.4 Hz), 1.83-2.07 (1H, m), 2.71-2.76 (2H, m), 2.89 (2H, s), 3.33-3.41 (2H, m), 3.69-3.75 (3H, m), 6.39-6.48 (2H, m), 6.66 (1H, dd, J=7.9, 1.9 Hz), 7.13 (1H, d, J=9.0 Hz), 7.23 (1H, t, J=8.1 Hz), 7.50 (1H, dd, J=8.9, 2.5 Hz), 7.73 (1H, s), 7.79 (1H, d, J=2.6 Hz), 7.93 (1H, t, J=4.9 Hz), 7.98 (1H, s), 9.38 (1H, s).

### Example 210

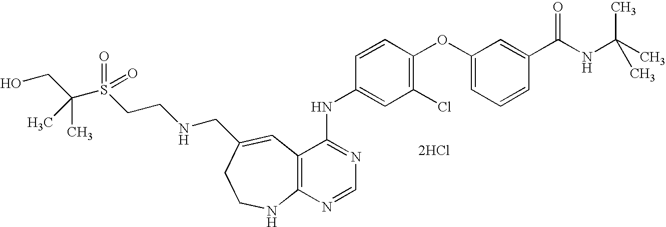

### Production of N-(tert-butyl)-3-[2-chloro-4-({6-[({2-[(2-hydroxy-1,1-dimethylethyl)sulfonyl]ethyl}amino)methyl]-8,9-dihydro-7H-pyrimido[4,5-b]azepin-4-yl}amino)phenoxy]benzamide dihydrochloride

To a suspension of N-(tert-butyl)-3-{2-chloro-4-[(6-formyl-8,9-dihydro-7H-pyrimido[4,5-b]azepin-4-yl)amino]phenoxy}benzamide (120 mg), 2-[(2-aminoethyl)sulfonyl]-2-methyl-1-propanol hydrochloride (106 mg) and triethylamine (68 μL) in tetrahydrofuran (10 mL) was added sodium triacetoxyborohydride (0.31 g) at room temperature. The mixture was stirred at room temperature for 2 days. Aqueous sodium bicarbonate solution was added to the reaction mixture, and the mixture was extracted with ethyl acetate. The organic layer was washed with water and saturated brine, dried over anhydrous magnesium sulfate and concentrated under reduced pressure. The residue was separated and purified by basic silica gel column chromatography (eluent, ethyl acetate→ethyl acetate:methanol=4:1) to give pale-yellow amorphous. To the solution of the obtained amorphous in ethanol (5.0 mL) was added 4N hydrochloric acid/ethyl acetate (0.5 mL) at room temperature. The mixture was concentrated under reduced pressure and crystallized from ethanol-ethyl acetate to give the title compound (67.4 mg) as pale-yellow crystals.

¹H-NMR (DMSO-d₆) δ: 1.26 (6H, s), 1.36 (9H, s), 2.57-2.64 (2H, m), 3.31-3.49 (4H, m), 3.60 (2H, s), 3.67-3.73 (2H, m), 3.75-3.88 (2H, m), 6.89 (1H, s), 7.08-7.12 (1H, m), 7.19 (1H, d, J=8.7 Hz), 7.30-7.35 (1H, m), 7.44 (1H, t, J=8.0 Hz), 7.55-7.63 (2H, m), 7.82 (1H, s), 7.85 (1H, d, J=2.7 Hz), 8.18 (1H, s), 8.20-8.38 (1H, m), 9.36-9.49 (2H, m), 9.72-9.91 (1H, m).

### Example 211

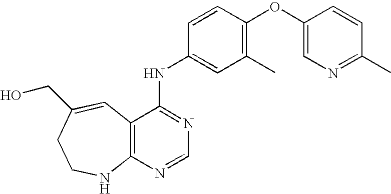

### Production of [4-({3-methyl-4-[(6-methylpyridin-3-yl)oxy]phenyl}amino)-8,9-dihydro-7H-pyrimido[4,5-b]azepin-6-yl]methanol

4-({3-Methyl-4-[(6-methylpyridin-3-yl)oxy]phenyl}amino)-8,9-dihydro-7H-pyrimido[4,5-b]azepine-6-carboxylic acid (1.00 g) was dissolved in tetrahydrofuran (20 mL) and cooled to −10° C.

Triethylamine (0.50 mL) and isobutyl chloroformate

(0.354 mL) were added to the solution, and the mixture was stirred at −10° C. for 1 hr. The insoluble material was filtrated off. A solution of sodium borohydride (192 mg) in water (15 mL) was added dropwise to the filtrate at −10° C., and the mixture was stirred for 2 hr. Saturated aqueous sodium hydrogencarbonate solution was added to the reaction mixture, and the mixture was extracted with ethyl acetate. The organic layer was washed with saturated brine, dried over anhydrous magnesium sulfate and concentrated under reduced pressure. The residue was separated and purified by silica gel column chromatography (eluent, hexane:ethyl acetate=10:90→0:100→ethyl acetate:methanol=90:10) and crystallized from ethyl acetate-diisopropyl ether to give the title compound (632 mg) as a white powder.

¹H-NMR (DMSO-d₆) δ: 2.12 (3H, s), 2.37-2.45 (2H, m), 2.42 (3H, s), 3.26-3.38 (2H, m), 4.01 (2H, d, J=5.5 Hz), 4.81 (1H, t, J=5.5 Hz), 6.46 (1H, s), 6.87 (1H, d, J=8.7 Hz), 7.10-7.25 (3H, m), 7.33-7.48 (2H, m), 7.86 (1H, s), 8.14 (1H, d, J=2.5 Hz), 8.51 (1H, s).

### Example 212

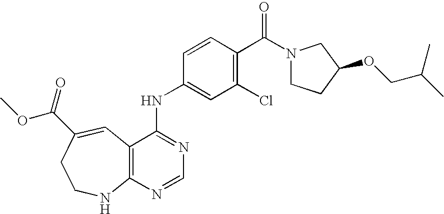

### Production of methyl 4-[(3-chloro-4-{[(3S)-3-isobutoxypyrrolidin-1-yl]carbonyl}phenyl)amino]-8,9-dihydro-7H-pyrimido[4,5-b]azepine-6-carboxylate

### (i) Production of tert-butyl (3S)-3-isobutoxypyrrolidine-1-carboxylate

Using tert-butyl (3S)-3-hydroxypyrrolidine-1-carboxylate (1.02 g), isobutyl bromide (1.00 g), tetra-n-butylammonium hydrogen sulfate (242 mg), toluene (10 mL) and 50% aqueous sodium hydroxide solution (2.5 mL), a similar reaction as in Example 168 (i) was carried out to give the title compound (670 mg) as a colorless oil.

¹H-NMR (CDCl₃) δ: 0.89 (6H, d, J=6.6 Hz), 1.46 (9H, s), 1.72-2.06 (3H, m), 3.09-3.23 (2H, m), 3.26-3.50 (4H, m), 3.92-4.05 (1H, m).

### (ii) Production of (3S)-3-isobutoxypyrrolidine hydrochloride

Using tert-butyl (3S)-3-isobutoxypyrrolidine-1-carboxylate (970 mg), 6N hydrochloric acid (3.5 mL) and tetrahydrofuran (20 mL), a similar reaction as in Example 168 (vii) was carried out to give the title compound (711 mg) as a brown oil.

¹H-NMR (DMSO-d₆) 0.86 (6H, d, J=6.8 Hz), 1.64-2.09 (3H, m), 3.02-3.28 (4H, m), 3.16 (2H, d, J=6.6 Hz), 4.07-4.22 (1H, m), 9.11 (1H, br s), 9.29 (1H, br s).

### (iii) Production of (3S)-1-(2-chloro-4-nitrobenzoyl)-3-isobutoxypyrrolidine

Using 2-chloro-4-nitrobenzoic acid (791 mg), thionyl chloride (0.50 mL), N,N-dimethylformamide (one drop), tetrahydrofuran (13 mL), (3S)-3-isobutoxypyrrolidine hydrochloride (707 mg) and triethylamine (1.7 mL), a similar reaction as in Example 192 (i) was carried out to give the title compound (819 mg) as a colorless oil.

¹H-NMR (CDCl₃) δ: 0.79-0.97 (6H, m), 1.70-2.22 (3H, m), 3.01-3.47 (4H, m), 3.59-3.92 (2H, m), 3.96-4.20 (1H, m), 7.52 (1H, d, J=8.5 Hz), 8.12-8.23 (1H, m), 8.30 (1H, d, J=2.1 Hz).

### (iv) Production of 3-chloro-4-{[(3S)-3-isobutoxypyrrolidin-1-yl]carbonyl}aniline

Using (3S)-1-(2-chloro-4-nitrobenzoyl)-3-isobutoxypyrrolidine (816 mg), reduced iron (703 mg), calcium chloride (143 mg), ethanol (27 mL) and water (3.0 mL), a similar reaction as in Example 192 (ii) was carried out to give the title compound (554 mg) as white crystals.

¹H-NMR (CDCl₃) δ: 0.82-0.97 (6H, m), 1.70-2.15 (3H, m), 3.02-3.33 (3H, m), 3.34-3.51 (1H, m), 3.61-3.79 (2H, m), 3.83 (2H, br s), 3.93-4.15 (1H, m), 6.53-6.60 (1H, m), 6.67 (1H, d, J=1.9 Hz), 7.09 (1H, d, J=8.3 Hz).

### (v) Production of methyl 4-[(3-chloro-4-{[(3S)-3-isobutoxypyrrolidin-1-yl]carbonyl}phenyl)amino]-8,9-dihydro-7H-pyrimido[4,5-b]azepine-6-carboxylate

Using methyl 4-chloro-8,9-dihydro-7H-pyrimido[4,5-b]azepine-6-carboxylate (122 mg), 3-chloro-4-{[(3S)-3-isobutoxypyrrolidin-1-yl]carbonyl}aniline (177 mg), pyridinium chloride (3.0 mg) and 1-methyl-2-pyrrolidone (1.5 mL), a similar reaction as in Example 192 (iii) was carried out to give the title compound (69.2 mg) as a white powder.

¹H-NMR (DMSO-d₆) δ: 0.78-0.91 (6H, m), 1.65-1.83 (1H, m), 1.86-2.05 (2H, m), 2.73 (2H, t, J=4.0 Hz), 3.01-3.65 (8H, m), 3.71 (3H, s), 3.96-4.15 (1H, m), 7.21-7.30 (1H, m), 7.53 (1H, dd, J=8.5, 2.1 Hz), 7.66-7.76 (2H, m), 7.96 (1H, t, J=4.9 Hz), 7.99-8.02 (1H, m), 9.46 (1H, s).

### Example 213

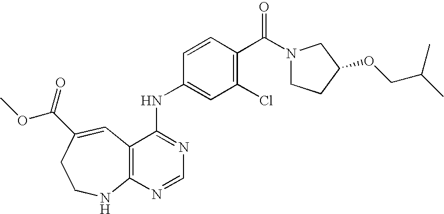

### Production of methyl 4-[(3-chloro-4-{[(3R)-3-isobutoxypyrrolidin-1-yl]carbonyl}phenyl)amino]-8,9-dihydro-7H-pyrimido[4,5-b]azepine-6-carboxylate

### (i) Production of tert-butyl (3R)-3-isobutoxypyrrolidine-1-carboxylate

Using tert-butyl (3R)-3-hydroxypyrrolidine-1-carboxylate (1.02 g), isobutyl bromide (1.6 mL), tetra-n-butylammonium hydrogen sulfate (216 mg), toluene (10 mL) and 50% aqueous sodium hydroxide solution (2.5 mL), a similar reaction as in Example 168 (i) was carried out to give the title compound (700 mg) as a colorless oil.

¹H-NMR (CDCl₃) δ: 0.89 (6H, d, J=6.6 Hz), 1.46 (9H, s), 1.73-2.05 (3H, m), 3.10-3.23 (2H, m), 3.27-3.50 (4H, m), 3.92-4.05 (1H, m).

### (ii) Production of (3R)-3-isobutoxypyrrolidine hydrochloride

Using tert-butyl (3R)-3-isobutoxypyrrolidine-1-carboxylate (1.00 g), 6N hydrochloric acid (3.5 mL) and tetrahydrofuran (20 mL), a similar reaction as in Example 168 (vii) was carried out to give the title compound (735 mg) as a brown oil.

¹H-NMR (DMSO-d₆) δ: 0.86 (6H, d, J=6.6 Hz), 1.65-2.10 (3H, m), 3.01-3.28 (4H, m), 3.16 (2H, d, J=6.6 Hz), 4.08-4.20 (1H, m), 9.16 (1H, br s), 9.39 (1H, br s).

### (iii) Production of (3R)-1-(2-chloro-4-nitrobenzoyl)-3-isobutoxypyrrolidine

Using 2-chloro-4-nitrobenzoic acid (820 mg), thionyl chloride (0.5 mL), N,N-dimethylformamide (one drop), tetrahydrofuran (13 mL), (3S)-3-isobutoxypyrrolidine hydrochloride (731 mg) and triethylamine (1.7 mL), a similar reaction as in Example 192 (i) was carried out to give the title compound (9.53 mg) as a colorless oil.

¹H-NMR (CDCl₃) δ: 0.79-0.98 (6H, m), 1.70-2.23 (3H, m), 2.99-3.47 (4H, m), 3.58-3.92 (2H, m), 3.95-4.22 (1H, m), 7.52 (1H, d, J=8.5 Hz), 8.10-8.27 (1H, m), 8.30 (1H, d, J=2.1 Hz).

### (iv) Production of 3-chloro-4-{[(3R)-3-isobutoxypyrrolidin-1-yl]carbonyl}aniline

Using (3R)-1-(2-chloro-4-nitrobenzoyl)-3-isobutoxypyrrolidine (947 mg), reduced iron (815 mg), calcium chloride (162 mg), ethanol (27 mL) and water (3.0 mL), a similar reaction as in Example 192 (ii) was carried out to give the title compound (633 mg) as white crystals.

¹H-NMR (CDCl₃) δ: 0.83-0.95 (6H, m), 1.68-2.17 (3H, m), 2.99-3.33 (3H, m), 3.33-3.50 (1H, m), 3.58-3.80 (2H, m), 3.83 (2H, br s), 3.92-4.16 (1H, m), 6.53-6.60 (1H, m), 6.67 (1H, d, J=2.3 Hz), 7.09 (1H, d, J=8.0 Hz).

### (v) Production of methyl 4-[(3-chloro-4-{[(3R)-3-isobutoxypyrrolidin-1-yl]carbonyl}phenyl)amino]-8,9-dihydro-7H-pyrimido[4,5-b]azepine-6-carboxylate

Using methyl 4-chloro-8,9-dihydro-7H-pyrimido[4,5-b]azepine-6-carboxylate (122 mg), 3-chloro-4-{[(3R)-3-isobutoxypyrrolidin-1-yl]carbonyl}aniline (181 mg), pyridinium chloride (3.0 mg) and 1-methyl-2-pyrrolidone (1.5 mL), a similar reaction as in Example 192 (iii) was carried out to give the title compound (170 mg) as a white powder.

¹H-NMR (DMSO-d₆) δ: 0.78-0.91 (6H, m), 1.62-1.84 (1H, m), 1.84-2.06 (2H, m), 2.73 (2H, t, J=4.2 Hz), 3.01-3.65 (8H, m), 3.71 (3H, s), 3.95-4.15 (1H, m), 7.21-7.29 (1H, m), 7.53 (1H, dd, J=8.3, 1.9 Hz), 7.68-7.75 (2H, m), 7.96 (1H, t, J=4.2 Hz), 7.99-8.02 (1H, m), 9.46 (1H, s).

### Example 214

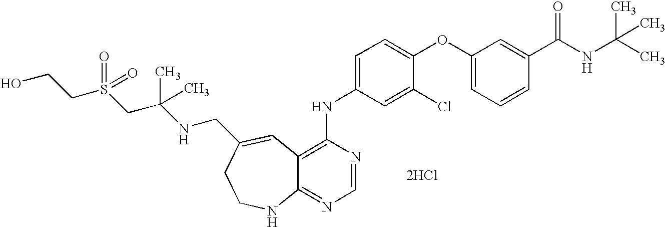

### Production of N-(tert-butyl)-3-[2-chloro-4-({6-[({2-[(2-hydroxyethyl)sulfonyl]-1,1-dimethylethyl}amino)methyl]-8,9-dihydro-7H-pyrimido[4,5-b]azepin-4-yl}amino)phenoxy]benzamide dihydrochloride

To a suspension of N-(tert-butyl)-3-{2-chloro-4-[(6-formyl-8,9-dihydro-7H-pyrimido[4,5-b]azepin-4-yl)amino]phenoxy}benzamide (120 mg), 2-[(2-amino-2-methylpropyl)sulfonyl]ethanol hydrochloride (106 mg) and triethylamine (68 μL) in tetrahydrofuran (15 mL) and N,N-dimethylformamide (5.0 mL), sodium triacetoxyborohydride (0.31 g) was added at room temperature. The mixture was stirred at room temperature for 2 days. Aqueous sodium bicarbonate solution was added to the reaction mixture, and the mixture was extracted with ethyl acetate. The organic layer was washed with water and saturated brine, dried over anhydrous magnesium sulfate and concentrated under reduced pressure. The residue was separated and purified by basic silica gel column chromatography (eluent, ethyl acetate→ethyl acetate:methanol=4:1) to give pale-yellow amorphous. 4N Hydrochloric acid/ethyl acetate (0.5 mL) was added to the solution of the obtained amorphous in ethanol (5.0 mL) at room temperature. The mixture was concentrated under reduced pressure and crystallized from ethanol-ethyl acetate to give the title compound (29.2 mg) as pale-yellow crystals.

¹H-NMR (DMSO-d₆) δ: 1.35 (9H, s), 1.62 (6H, s), 2.55-2.66 (2H, m), 3.32-3.87 (10H, m), 6.86 (1H, s), 7.04-7.10 (1H, m), 7.18 (1H, d, J=8.4 Hz), 7.26-7.31 (1H, m), 7.42 (1H, t, J=7.8 Hz), 7.53-7.66 (2H, m), 7.78-8.02 (3H, m), 8.09 (1H, s), 8.93-9.10 (2H, m).

### Example 215

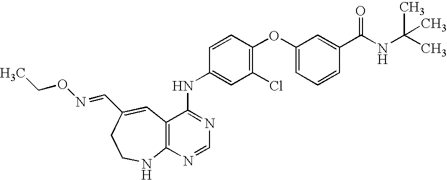

### Production of N-(tert-butyl)-3-[2-chloro-4-({6-[(ethoxyimino)methyl]-8,9-dihydro-7H-pyrimido[4,5-b]azepin-4-yl}amino)phenoxy]benzamide

A suspension of N-(tert-butyl)-3-{2-chloro-4-[(6-formyl-8,9-dihydro-7H-pyrimido[4,5-b]azepin-4-yl)amino]phenoxy}benzamide (120 mg), O-ethylhydroxylamine hydrochloride (117 mg) and sodium acetate (98.4 mg) in ethanol (15 mL) was stirred at room temperature for 3 days. Water was added to the reaction mixture, and the mixture was extracted with ethyl acetate. The organic layer was washed with water and saturated brine, dried over anhydrous magnesium sulfate and concentrated under reduced pressure. The residue was separated and purified by silica gel column chromatography (eluent, hexane:ethyl acetate=1:1→ethyl acetate) to give the title compound (80.5 mg) as pale-yellow crystals.

¹H-NMR (CDCl₃) δ: 1.30 (3H, t, J=7.1 Hz), 1.46 (9H, s), 2.87 (2H, t, J=4.8 Hz), 3.53 (2H, q, J=4.8 Hz), 4.16 (2H, q, J=7.2 Hz), 5.65-5.73 (1H, m), 5.91 (1H, br s), 6.40 (1H, s), 6.57 (1H, s), 6.99 (1H, d, J=9.0 Hz), 7.02-7.05 (1H, m), 7.25-7.40 (4H, m), 7.67 (1H, d, J=2.7 Hz), 7.80 (1H, s), 8.10 (1H, s).

### Example 216

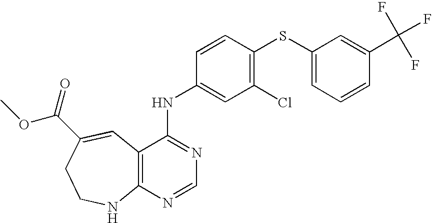

### Production of methyl 4-[(3-chloro-4-{[3-(trifluoromethyl)phenyl]thio}phenyl)amino]-8,9-dihydro-7H-pyrimido[4,5-b]azepine-6-carboxylate

### (i) Production of 2-chloro-4-nitro-1-{[3-(trifluoromethyl)phenyl]thio}benzene

A mixture of 2-chloro-1-fluoro-4-nitrobenzene (1.98 g), 3-(trifluoromethyl)benzenethiol (2.01 g), potassium carbonate (2.04 g) and N,N-dimethylformamide (20 mL) was stirred at room temperature for 59 hr. Aqueous sodium bicarbonate solution was added to the reaction mixture, and the mixture was extracted with ethyl acetate. The organic layer was washed with saturated brine, dried over anhydrous magnesium sulfate and concentrated under reduced pressure. The residue was separated and purified by silica gel column chromatography (eluent, hexane:ethyl acetate=100:0→95:5) to give the title compound (3.64 g) as pale-yellow amorphous.

¹H-NMR (CDCl₃) δ: 6.80 (1H, d, J=9.1 Hz), 7.64 (1H, t, J=7.8 Hz), 7.77 (2H, t, J=7.6 Hz), 7.84 (1H, s), 7.94 (1H, dd, J=8.9, 2.5 Hz), 8.26 (1H, d, J=2.7 Hz).

### (ii) Production of 3-chloro-4-{[3-(trifluoromethyl)phenyl]thio}aniline

The mixture of 2-chloro-4-nitro-1-{[3-(trifluoromethyl)phenyl]thio}benzene (3.64 g), reduced iron (3.38 g), calcium chloride (688 mg) and 15% water-containing ethanol (120 mL) was stirred at 100° C. for 15 hr. Iron was removed by filtration, and the solvent was evaporated under reduced pressure. Aqueous sodium bicarbonate solution was added to the residue, and the mixture was extracted with ethyl acetate. The organic layer was washed with saturated brine, dried over anhydrous magnesium sulfate and concentrated under reduced pressure. The residue was separated and purified by silica gel column chromatography (eluent, hexane:ethyl acetate=95:5→75:25) to give the title compound (2.07 g) as colorless amorphous.

¹H-NMR (CDCl₃) δ: 3.94 (2H, s), 6.58 (1H, dd, J=8.3, 2.7 Hz), 6.84 (1H, d, J=2.7 Hz), 7.16-7.24 (2H, m), 7.28-7.40 (3H, m).

### (iii) Production of methyl 4-[(3-chloro-4-{[3-(trifluoromethyl)phenyl]thio}phenyl)amino]-8,9-dihydro-7H-pyrimido[4,5-b]azepine-6-carboxylate

A mixture of methyl 4-chloro-8,9-dihydride-7H-pyrimido[4,5-b]azepine-6-carboxylate (102 mg), 3-chloro-4-{[3-(trifluoromethyl)phenyl]thio}aniline (142 mg), pyridinium chloride (17.0 mg) and 1-methyl-2-pyrrolidone (5.0 mL) was stirred at 120° C. for 18 hr. Aqueous sodium bicarbonate solution was added to the reaction mixture, and the mixture was extracted with ethyl acetate. The organic layer was washed with saturated brine, dried over anhydrous magnesium sulfate and concentrated under reduced pressure. The residue was separated and purified by silica gel column chromatography (eluent, hexane:ethyl acetate=100:0→50:50) to give the title compound (69.2 mg) as colorless crystals.

¹H-NMR (DMSO-d₆) δ: 2.71-2.77 (2H, m), 2.89 (1H, s), 3.38 (2H, q, J=4.4 Hz), 3.72 (3H, s), 7.33-7.41 (1H, m), 7.47 (1H, s), 7.54-7.60 (4H, m), 7.72 (1H, s), 7.93 (1H, d, J=2.3 Hz), 8.03 (1H, s), 9.58 (1H, s).

### Example 217

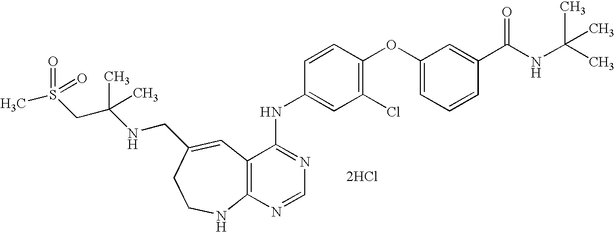

### Production of N-(tert-butyl)-3-(2-chloro-4-{[6-({[1,1-dimethyl-2-(methylsulfonyl)ethyl]amino}methyl)-8,9-dihydro-7H-pyrimido[4,5-b]azepin-4-yl]amino}phenoxy)benzamide dihydrochloride

A suspension of N-(tert-butyl)-3-{2-chloro-4-[(6-formyl-8,9-dihydro-7H-pyrimido[4,5-b]azepin-4-yl)amino]phenoxy}benzamide (120 mg), 2-methyl-1-(methylsulfonyl)-2-propylamine hydrochloride (90 mg) and triethylamine (68 μL) in tetrahydrofuran (15 mL) and N,N-dimethylformamide (5.0 mL) was stirred at room temperature for 1 hr. Sodium triacetoxyborohydride (0.31 g) was added to the suspension at room temperature, and the mixture was stirred for 2 days. Aqueous sodium bicarbonate solution was added to the reaction mixture, and the mixture was extracted with ethyl acetate. The organic layer was washed with water and saturated brine, dried over anhydrous magnesium sulfate and concentrated under reduced pressure. The residue was separated and purified by basic silica gel column chromatography (eluent, ethyl acetate-methyl acetate:methanol=4:1) to give pale-yellow amorphous. 4N Hydrochloric acid/ethyl acetate (0.5 mL) was added to the solution of the obtained amorphous in ethanol (1.0 mL) at room temperature. The mixture was concentrated under reduced pressure and crystallized from ethanol-ethyl acetate to give the title compound (100 mg), as pale-yellow crystals.

¹H-NMR (DMSO-d₆) δ: 1.36 (9H, s), 1.64 (6H, s), 2.58-2.66 (2H, m), 3.15 (3H, s), 3.40-3.49 (2H, m), 3.71-3.80 (2H, m), 3.83 (2H, s), 6.92 (1H, s), 7.08 (1H, dd, J=7.2, 2.7 Hz), 7.17 (1H, d, J=8.7 Hz), 7.29-7.32 (1H, m), 7.40-7.45 (1H, m), 7.55-7.66 (2H, m), 7.81 (1H, s), 7.91 (1H, d, J=2.4 Hz), 8.09-8.14 (2H, m), 9.21-9.34 (2H, m), 9.76-9.97 (1H, m).

### Example 218

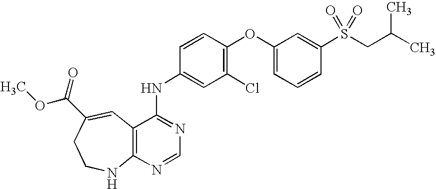

### Production of methyl 4-({3-chloro-4-[3-(isobutylsulfonyl)phenoxy]phenyl}amino)-8,9-dihydro-7H-pyrimido[4,5-b]azepine-6-carboxylate

Using methyl 4-chloro-8,9-dihydro-7H-pyrimido[4,5-b]azepine-6-carboxylate (200 mg), 3-chloro-4-[3-(isobutylsulfonyl)phenoxy]aniline (314 mg), pyridinium chloride (9.6 mg) and 1-methyl-2-pyrrolidone (5.0 mL), a similar reaction as in Example 202 was carried out to give the title compound (335 mg) as pale-yellow crystals.

¹H-NMR (CDCl₃) δ: 1.06 (6H, d, J=6.3 Hz), 2.18-2.29 (1H, m), 2.91 (2H, t, J=4.8 Hz), 2.98 (2H, d, J=6.3 Hz), 3.51-3.56 (2H, m), 3.83 (3H, s), 5.78-5.85 (1H, m), 6.83 (1H, s), 7.09 (1H, d, J=9.0 Hz), 7.19-7.23 (1H, m), 7.40 (1H, dd, J=9.0, 2.7 Hz), 7.43-7.45 (1H, m), 7.48-7.53 (1H, m), 7.59-7.63 (1H, m), 7.68 (1H, s), 7.78 (1H, s), 8.13 (1H, s).

### Example 219

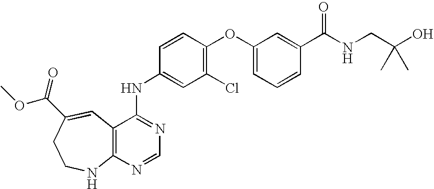

### Production of methyl 4-{[3-chloro-4-(3-{[(2-hydroxy-2-methylpropyl)amino]carbonyl}phenoxy)phenyl]amino}-8,9-dihydro-7H-pyrimido[4,5-b]azepine-6-carboxylate

### (i) Production of 3-(2-chloro-4-nitrophenoxy)-N-(2-hydroxy-2-methylpropyl)benzamide

Using 3-(2-chloro-4-nitrophenoxy)benzoic acid (500 mg), 1-amino-2-methylpropan-2-ol (199 mg), tetrahydrofuran (5.0 mL), N,N-dimethylformamide (5.0 mL), triethylamine (0.70 mL), 1-hydroxybenzotriazole (347 mg) and 1-ethyl-3-(3-dimethylaminopropyl)carbodiimide hydrochloride (496 mg), a similar reaction as in Example 168 (viii) was carried out to give the title compound (448 mg) as a white powder.

¹H-NMR (CDCl₃) δ: 1.30 (6H, s), 3.48 (2H, d, J=5.8 Hz), 6.60 (1H, br s), 6.92 (1H, d, J=9.1 Hz), 7.19-7.25 (1H, m), 7.47-7.58 (2H, m), 7.61-7.69 (1H, m), 8.07 (1H, dd, J=9.1, 2.8 Hz), 8.39 (1H, d, J=2.8 Hz).

### (ii) Production of 3-(4-amino-2-chlorophenoxy)-N-(2-hydroxy-2-methylpropyl)benzamide

Using 3-(2-chloro-4-nitrophenoxy)-N-(2-hydroxy-2-methylpropyl)benzamide (446 mg), ethanol (13.5 mL), water (1.5 mL), reduced iron (347 mg) and calcium chloride (68.4 mg), a similar reaction as in Example 192 (ii) was carried out to give the title compound (349 mg) as a colorless oil.

¹H-NMR (CDCl₃) δ: 1.27 (6H, s), 2.38 (1H, br s), 3.44 (2H, d, J=6.0 Hz), 3.71 (2H, br s), 6.53-6.64 (1H, m), 6.57 (1H, dd, J=8.7, 2.8 Hz), 6.78 (1H, d, J=2.8 Hz), 6.90 (1H, d, J=8.7 Hz), 6.97-7.03 (1H, m), 7.29-7.37 (2H, m), 7.38-7.44 (1H, m).

### (iii) Production of methyl 4-{[3-chloro-4-(3-{[(2-hydroxy-2-methylpropyl)amino]carbonyl}phenoxy)phenyl]amino}-8,9-dihydro-7H-pyrimido[4,5-b]azepine-6-carboxylate

Using methyl 4-chloro-8,9-dihydro-7H-pyrimido[4,5-b]azepine-6-carboxylate (110 mg), 3-(4-amino-2-chlorophenoxy)-N-(2-hydroxy-2-methylpropyl)benzamide (197 mg), pyridinium chloride (3.0 mg) and 1-methyl-2-pyrrolidone (1.5 mL), a similar reaction as in Example 192 (iii) was carried out to give the title compound (181 mg) as a pale-orange powder.

¹H-NMR (DMSO-d₆) δ: 1.09 (6H, s), 2.70-2.78 (2H, m), 3.23 (2H, d, J=6.2 Hz), 3.34-3.42 (2H, m), 3.72 (3H, s), 4.53 (1H, s), 7.07 (1H, ddd, J=8.0, 2.3, 1.0 Hz), 7.16 (1H, d, J=9.0 Hz), 7.40 (1H, dd, J=2.3, 1.0 Hz), 7.45 (1H, t, J=8.0 Hz), 7.52 (1H, dd, J=9.0, 2.5 Hz), 7.61 (1H, d, J=8.0 Hz), 7.74 (1H, s), 7.82 (1H, d, J=2.5 Hz), 7.92 (1H, t, J=5.0 Hz), 7.98 (1H, s), 8.30 (1H, t, J=6.1 Hz), 9.40 (1H, s).

### Example 220

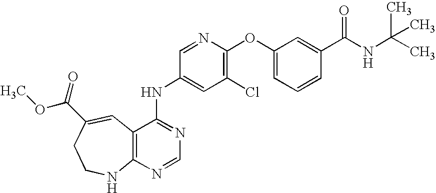

### Production of methyl 4-[(6-{3-[(tert-butylamino)carbonyl]phenoxy}-5-chloropyridin-3-yl)amino]-8,9-dihydro-7H-pyrimido[4,5-b]azepine-6-carboxylate

Using methyl 4-chloro-8,9-dihydro-7H-pyrimido[4,5-b]azepine-6-carboxylate (100 mg), 3-[(5-amino-3-chloropyridin-2-yl)oxy]-N-(tert-butyl)benzamide (133 mg), pyridinium chloride (4.8 mg) and 1-methyl-2-pyrrolidone (2.0 mL), a similar reaction as in Example 202 was carried out to give the title compound (163.5 mg) as pale-yellow crystals.

¹H-NMR (CDCl₃) δ: 1.46 (9H, s), 2.90 (2H, t, J=4.5 Hz), 3.50-3.54 (2H, m), 3.82 (3H, s), 5.76-5.85 (1H, m), 5.88-5.96 (1H, m), 6.83 (1H, s), 7.21-7.29 (1H, m), 7.42-7.48 (2H, m), 7.54-7.58 (1H, m), 7.65 (1H, s), 8.03 (1H, d, J=2.7 Hz), 8.07 (1H, s), 8.16 (1H, d, J=2.7 Hz).

### Example 221

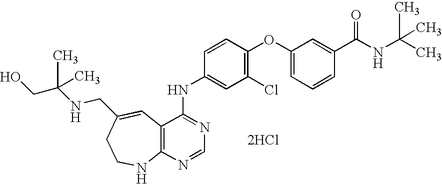

### Production of N-(tert-butyl)-3-{2-chloro-4-[(6-{[(2-hydroxy-1,1-dimethylethyl)amino]methyl}-8,9-dihydro-7H-pyrimido[4,5-b]azepin-4-yl)amino]phenoxy}benzamide dihydrochloride

To a solution of N-(tert-butyl)-3-{2-chloro-4-[(6-formyl-8,9-dihydro-7H-pyrimido[4,5-b]azepin-4-yl)amino]phenoxy}benzamide (120 mg) and 2-amino-2-methyl-1-propanol (42.8 mg) in tetrahydrofuran (5.0 mL) was added sodium triacetoxyborohydride (0.31 g) at room temperature. The mixture was stirred at room temperature for 20 hr. Aqueous sodium bicarbonate solution was added to the reaction mixture, and the mixture was extracted with ethyl acetate. The organic layer was washed with water and saturated brine, dried over anhydrous magnesium sulfate and concentrated under reduced pressure. The residue was separated and purified by basic silica gel column chromatography (eluent, ethyl acetate-methyl acetate:methanol=4:1) to give pale-yellow amorphous. 4N Hydrochloric acid/ethyl acetate (0.5 mL) was added to a solution of the obtained amorphous in ethanol (5.0 mL) at room temperature. The mixture was concentrated under reduced pressure and crystallized from ethanol-ethyl acetate to give the title compound (102 mg) as yellow crystals.

¹H-NMR (DMSO-d₆) δ: 1.31 (6H, s), 1.36 (9H, s), 2.55-2.63 (2H, m), 3.40-3.50 (2H, m), 3.51 (2H, s), 3.62-3.73 (2H, m), 6.89 (1H, s), 7.04-7.10 (1H, m), 7.17 (1H, d, J=8.7 Hz), 7.28-7.34 (1H, m), 7.40-7.45 (1H, m), 7.52-7.64 (2H, m), 7.81 (1H, s), 7.86-7.91 (1H, m), 8.08-8.24 (2H, m), 8.64-8.80 (2H, m), 9.73-9.99 (1H, m).

### Example 222

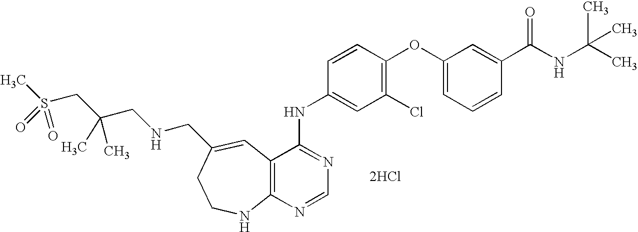

### Production of N-(tert-butyl)-3-(2-chloro-4-{[6-({[2,2-dimethyl-3-(methylsulfonyl)propyl]amino}methyl)-8,9-dihydro-7H-pyrimido[4,5-b]azepin-4-yl]amino}phenoxy)benzamide dihydrochloride

Using N-(tert-butyl)-3-{2-chloro-4-[(6-formyl-8,9-dihydro-7H-pyrimido[4,5-b]azepin-4-yl)amino]phenoxy}benzamide (120 mg), 2,2-dimethyl-3-(methylsulfonyl)-1-propylamine hydrochloride (97 mg), triethylamine (0.134 mL), sodium triacetoxyborohydride (0.31 g), tetrahydrofuran (5.0 mL), N,N-dimethylformamide (1.0 mL), ethanol (5.0 mL) and 4N hydrochloric acid/ethyl acetate (0.5 mL), a similar reaction as in Example 217 was carried out to give the title compound (50.5 mg) as yellow crystals.

¹H-NMR (DMSO-d₆) δ: 1.25 (6H, s), 1.36 (9H, s), 2.60-2.66 (2H, m), 3.01 (3H, s), 3.06-3.16 (2H, m), 3.41-3.49 (2H, m), 3.56 (2H, s), 3.71-3.81 (2H, m), 6.95 (1H, s), 7.09 (1H, dd, J=8.1, 2.7 Hz), 7.18 (1H, d, J=9.0 Hz), 7.31-7.32 (1H, m), 7.40-7.45 (1H, m), 7.53-7.62 (2H, m), 7.81 (1H, s), 7.86 (1H, d, J=2.7 Hz), 8.18 (1H, s), 8.20-8.38 (1H, m), 8.86-9.04 (2H, m), 9.82-10.00 (1H, m).

### Example 223

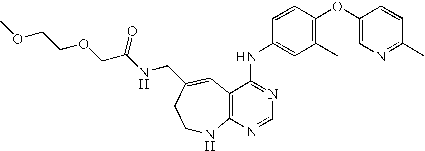

### Production of 2-(2-methoxyethoxy)-N-{[4-({3-methyl-4-[(6-methylpyridin-3-yl)oxy]phenyl}amino)-8,9-dihydro-7H-pyrimido[4,5-b]azepin-6-yl]methyl}acetamide

### (i) Production of 6-(azidomethyl)-N-{3-methyl-4-[(6-methylpyridin-3-yl)oxy]phenyl}-8,9-dihydro-7H-pyrimido[4,5-b]azepin-4-amine

[4-({3-Methyl-4-[(6-methylpyridin-3-yl)oxy]phenyl}amino)-8,9-dihydro-7H-pyrimido[4,5-b]azepin-6-yl]methanol (201 mg) was dissolved in tetrahydrofuran (2.0 mL). Diphenylphosphorylazide (0.166 mL) and diazabicyclo[5,4,0]-7-undecene (0.229 mL) were added to the solution, and the mixture was stirred at room temperature for 1 hr. Water was added to the reaction mixture, and the mixture was extracted with ethyl acetate. The organic layer was dried over anhydrous magnesium sulfate and concentrated under reduced pressure. The residue was separated and purified by silica gel column chromatography (eluent, hexane:ethyl acetate=33:67→0:100→ethyl acetate:methanol=95:5) to give the title compound (198 mg) as a pale-yellow oil.

¹H-NMR (CDCl₃) δ: 2.23 (3H, s), 2.52 (3H, s), 2.55-2.64 (2H, m), 3.47-3.58 (2H, m), 3.91 (2H, s), 5.88 (1H, t, J=8.7 Hz), 6.30 (1H, s), 6.61 (1H, s), 6.86 (1H, d, J=8.7 Hz), 7.03-7.13 (2H, m), 7.23 (1H, dd, J=8.7, 2.6 Hz), 7.32 (1H, d, J=2.6 Hz), 8.08 (1H, s), 8.25 (1H, dd, J=2.5, 0.9 Hz).

### (ii) Production of tert-butyl {[4-({3-methyl-4-[(6-methylpyridin-3-yl)oxy]phenyl}amino)-8,9-dihydro-7H-pyrimido[4,5-b]azepin-6-yl]methyl}carbamate

Using 6-(azidomethyl)-N-{3-methyl-4-[(6-methylpyridin-3-yl)oxy]phenyl}-8,9-dihydro-7H-pyrimido[4,5-b]azepin-4-amine (195 mg), triphenylphosphine (138 mg), tetrahydrofuran (2.0 mL), water (0.20 mL), di-tert-butyl dicarbonate (0.35 mL), triethylamine (0.35 mL) and methanol (3.0 mL), a similar reaction as in Example 168 (iv) was carried out to give the title compound (160 mg) as white crystals.

¹H-NMR (CDCl₃) δ: 1.46 (9H, s), 2.22 (3H, s), 2.47-2.57 (5H, m), 3.45-3.56 (2H, m), 3.85 (2H, d, J=6.0 Hz), 4.75 (1H, br s), 5.44 (1H, t, J=4.1 Hz), 6.18 (1H, s), 6.50 (1H, s), 6.86 (1H, d, J=8.7 Hz), 7.02-7.14 (2H, m), 7.21-7.30 (1H, m), 7.33 (1H, d, J=2.6 Hz), 8.08 (1H, s), 8.25 (1H, dd, J=2.6 Hz, 0.9 Hz),

### (iii) Production of 6-(aminomethyl)-N-{3-methyl-4-[(6-methylpyridin-3-yl)oxy]phenyl}-8,9-dihydro-7H-pyrimido[4,5-b]azepin-4-amine trihydrochloride

Using tert-butyl {[4-({3-methyl-4-[(6-methylpyridin-3-yl)oxy]phenyl}amino)-8,9-dihydro-7H-pyrimido[4,5-b]azepin-6-yl]methyl}carbamate (158 mg), 6N hydrochloric acid (0.50 mL) and ethanol (3.0 mL), a similar reaction as in Example 181 (viii) was carried out to give the title compound (152 mg) as a white powder.

¹H-NMR (DMSO-d₆) δ: 2.20 (3H, s), 2.54-2.64 (2H, m), 2.61 (3H, s), 3.46-3.54 (2H, m), 3.59-3.69 (2H, m), 6.77 (1H, s), 7.08 (1H, d, J=8.7 Hz), 7.38 (1H, dd, J=8.7, 2.5 Hz), 7.49 (1H, d, J=2.1 Hz), 7.66 (1H, d, J=8.9 Hz), 7.79 (1H, dd, J=8.9, 2.1 Hz), 8.22 (1H, s), 8.39 (1H, d, J=2.5 Hz), 8.54 (3H, br s), 8.70 (1H, br s), 10.23 (1H, br s).

### (iv) Production of 2-(2-methoxyethoxy)-N-{[4-({3-methyl-4-[(6-methylpyridin-3-yl)oxy]phenyl}amino)-8,9-dihydro-7H-pyrimido[4,5-b]azepin-6-yl]methyl}acetamide

Using 6-(aminomethyl)-N-{3-methyl-4-[(6-methylpyridin-3-yl)oxy]phenyl}-8,9-dihydro-7H-pyrimido[4,5-b]azepin-4-amine trihydrochloride (72.1 mg), (2-methoxyethoxy)acetic acid (0.0328 mL), triethylamine (0.2 mL), 1-hydroxybenzotriazole (43.1 mg), 1-ethyl-3-(3-dimethylaminopropyl)carbodiimide hydrochloride (56.0 mg), tetrahydrofuran (0.50 mL) and N,N-dimethylformamide (0.50 mL), a similar reaction as in Example 168 (viii) was carried out to give the title compound (49.5 mg) as pale-orange crystals.

¹H-NMR (DMSO-d₆) δ: 2.13 (3H, s), 2.38 (2H, t, J=4.3 Hz), 2.42 (3H, s), 3.21 (3H, s), 3.25-3.36 (2H, m), 3.40-3.47 (2H, m), 3.54-3.63 (2H, m), 3.86-3.95 (2H, m), 3.92 (2H, s), 6.43 (1H, s), 6.87 (1H, d, J=8.7 Hz), 7.14 (1H, dd, J=8.7, 3.0 Hz), 7.18-7.26 (2H, m), 7.38 (1H, dd, J=8.6, 2.5 Hz), 7.44 (1H, d, J=2.5 Hz), 7.78 (1H, t, J=6.0 Hz), 7.87 (1H, s), 8.14 (1H, d, J=3.0 Hz), 8.54 (1H, s).

### Example 224

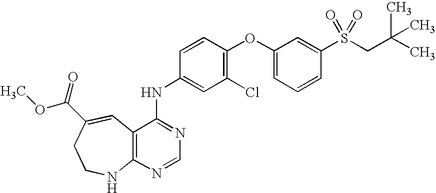

### Production of methyl 4-[(3-chloro-4-{3-[(2,2-dimethylpropyl)sulfonyl]phenoxy}phenyl)amino]-8,9-dihydro-7H-pyrimido[4,5-b]azepine-6-carboxylate

Using methyl 4-chloro-8,9-dihydro-7H-pyrimido[4,5-b]azepine-6-carboxylate (100 mg), 3-chloro-4-{3-[(2,2-dimethylpropyl)sulfonyl]phenoxy}aniline (162 mg), pyridinium chloride (4.8 mg) and 1-methyl-2-pyrrolidone (5.0 mL), a similar reaction as in Example 202 was carried out to give the title compound (129 mg) as pale-yellow crystals.

¹H-NMR (CDCl₃) δ: 1.18 (9H, s), 2.91 (2H, t, J=4.7 Hz), 3.03 (2H, s), 3.53 (2H, q, J=4.7 Hz), 3.83 (3H, s), 5.82 (1H, t, J=4.7 Hz), 6.82 (1H, s), 7.06 (1H, d, J=8.7 Hz), 7.13-7.20 (1H, m), 7.38 (1H, dd, J=8.7, 2.7 Hz), 7.45-7.51 (2H, m), 7.59-7.62 (1H, m), 7.67 (1H, s), 7.76 (1H, d, J=2.7 Hz), 8.12 (1H, s).

### Example 225

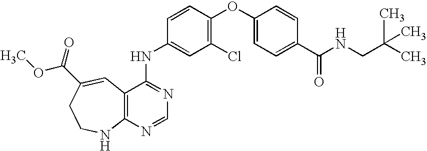

### Production of methyl 4-{[3-chloro-4-(4-{[(2,2-dimethylpropyl)amino]carbonyl}phenoxy)phenyl]amino}-8,9-dihydro-7H-pyrimido[4,5-b]azepine-6-carboxylate

### (i) Production of methyl 4-(2-chloro-4-nitrophenoxy)benzoate

A mixture of 3-chloro-4-fluoro-1-nitrobenzene (2.0 g), methyl 4-hydroxybenzoate (1.82 g) and potassium carbonate (1.65 g) in N,N-dimethylformamide (40 mL) was stirred at room temperature for 20 hr. Water was added to the reaction mixture, and the mixture was extracted with ethyl acetate. The organic layer was washed with water and saturated brine, dried over anhydrous magnesium sulfate and concentrated under reduced pressure. The residue was separated and purified by silica gel column chromatography (eluent, hexane:ethyl acetate=19:1→7:3→1:1) and basic silica gel column chromatography (eluent, hexane:ethyl acetate=3:1) to give the title compound (3.48 g) as colorless crystals.

¹H-NMR (CDCl₃) δ: 3.93 (3H, s), 7.01-7.10 (3H, m), 8.08-8.13 (3H, m), 8.40 (1H, d, J=3.0 Hz).

### (ii) Production of 4-(2-chloro-4-nitrophenoxy)benzoic acid

To a solution of methyl 4-(2-chloro-4-nitrophenoxy)benzoate (3.40 g) in tetrahydrofuran (50 mL) and isopropyl alcohol (25 mL) was added 1N aqueous sodium hydroxide solution (13 mL) at room temperature. The mixture was stirred at room temperature for 16 hr, and extracted with diethyl ether. The aqueous layer was acidified with 1N hydrochloric acid and extracted with ethyl acetate. The organic layer was washed with water and saturated brine, dried over anhydrous magnesium sulfate and concentrated under reduced pressure. The precipitated crystals were collected by filtration, and washed with diisopropyl ether to give the title compound (2.79 g) as colorless crystals.

¹H-NMR (CDCl₃) δ: 7.07-7.11 (3H, m), 8.12-8.18 (3H, m), 8.42 (1H, d, J=2.7 Hz).

### (iii) Production of 4-(2-chloro-4-nitrophenoxy)-N-(2,2-dimethylpropyl)benzamide

To a solution of 4-(2-chloro-4-nitrophenoxy)benzoic acid (1.5 g) and N,N-dimethylformamide (one drop) in tetrahydrofuran (15 mL) was added thionyl chloride (0.56 mL) at room temperature. The mixture was stirred at room temperature for 1 hr, and concentrated under reduced pressure. A solution of the residue in tetrahydrofuran (39 mL) was added dropwise to a solution of 2,2-dimethyl-1-propylamine (0.67 g) and triethylamine (2.1 mL) in tetrahydrofuran (5.0 mL) at 0° C. The mixture was stirred at room temperature for 3 days. Water was added to the reaction mixture, and the mixture was extracted with ethyl acetate. The organic layer was washed with water and saturated brine, dried over anhydrous magnesium sulfate and concentrated under reduced pressure. The residue was separated and purified by silica gel column chromatography (eluent, hexane:ethyl acetate=9:1→3:2→1:4) to give the title compound (1.88 g) as colorless crystals.

¹H-NMR (CDCl₃) δ: 1.00 (9H, s), 3.30 (2H, d, J=6.3 Hz), 6.04-6.15 (1H, m), 6.98 (1H, d, J=9.0 Hz), 7.11 (2H, d, J=8.9 Hz), 7.85 (2H, d, J=8.9 Hz), 8.10 (1H, dd, J=9.0, 2.7 Hz), 8.41 (1H, d, J=2.7 Hz).

### (iv) Production of 4-(4-amino-2-chlorophenoxy)-N-(2,2-dimethylpropyl)benzamide

Using 4-(2-chloro-4-nitrophenoxy)-N-(2,2-dimethylpropyl)benzamide (1.80 g), reduced iron (1.39 g), calcium chloride (0.28 g) and 15% water-containing ethanol (50 mL), a similar reaction as in Example 135.

### (iii) was carried out to give the title compound (1.18 g) as pale-yellow amorphous

¹H-NMR (CDCl₃) δ: 0.97 (9H, s), 3.26 (2H, d, J=6.3 Hz), 3.71 (2H, br s), 5.99-6.09 (1H, m), 6.58 (1H, dd, J=8.4, 2.7 Hz), 6.78 (1H, d, J=2.7 Hz), 6.86-6.93 (3H, m), 7.70 (2H, d, J=8.7 Hz).

### (v) Production of methyl 4-{[3-chloro-4-(4-{[(2,2-dimethylpropyl)amino]carbonyl}phenoxy)phenyl]amino}-8,9-dihydro-7H-pyrimido[4,5-b]azepine-6-carboxylate

Using methyl 4-chloro-8,9-dihydro-7H-pyrimido[4,5-b]azepine-6-carboxylate (200 mg), 4-(4-amino-2-chlorophenoxy)-N-(2,2-dimethylpropyl)benzamide (363 mg), pyridinium chloride (9.6 mg) and 1-methyl-2-pyrrolidone (5.0 mL), a similar reaction as in Example 202 was carried out to give the title compound (362 mg) as pale-yellow crystals.

¹H-NMR (CDCl₃) δ: 0.98 (9H, s), 2.90 (2H, t, J=4.8 Hz), 3.27 (2H, d, J=6.3 Hz), 3.53 (2H, q, J=4.8 Hz), 3.82 (3H, s), 5.79-5.85 (1H, m), 6.02-6.12 (1H, m), 6.84 (1H, s), 6.97 (2H, d, J=8.7 Hz), 7.07 (1H, d, J=8.7 Hz), 7.37 (1H, dd, J=8.7, 2.4 Hz), 7.66 (1H, s), 7.71-7.75 (3H, m), 8.12 (1H, s).

### Example 226

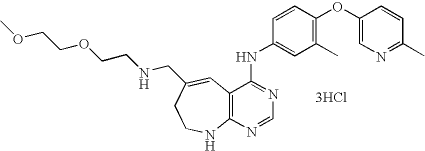

### Production of 6-({[2-(2-methoxyethoxy)ethyl]amino}methyl)-N-{3-methyl-4-[(6-methylpyridin-3-yl)oxy]phenyl}-8,9-dihydro-7H-pyrimido[4,5-b]azepin-4-amine trihydrochloride

### (i) Production of 4-({3-methyl-4-[(6-methylpyridin-3-yl)oxy]phenyl}amino)-8,9-dihydro-7H-pyrimido[4,5-b]azepin-6-carbaldehyde

[4-({3-Methyl-4-[(6-methylpyridin-3-yl)oxy]phenyl}amino)-8,9-dihydro-7H-pyrimido[4,5-b]azepin-6-yl]methanol (628 mg) was dissolved in a mixture of acetone (20 mL) and N,N-dimethylformamide (2.0 mL). Manganese dioxide (3.37 g) was added to the solution, and the mixture was stirred at room temperature for 1 day. The reaction mixture was filtered through Celite, and the filtrate was concentrated under reduced pressure. The residue was separated and purified by silica gel column chromatography (eluent, hexane:ethyl acetate=20:80→0:100→ethyl acetate:methanol=90:10) to give the title compound (242 mg) as a yellow powder.

¹H-NMR (CDCl₃) δ: 2.26 (3H, s), 2.53 (3H, s), 2.76-2.84 (2H, m), 3.46-3.55 (2H, m), 6.02 (1H, t, J=4.6 Hz), 6.81 (1H, s), 6.87 (1H, d, J=8.5 Hz), 7.05-7.16 (2H, m), 7.18-7.30 (2H, m), 7.34 (1H, d, J=2.5 Hz), 8.13 (1H, s), 8.25 (1H, d, J=2.6 Hz), 9.43 (1H, s).

### (ii) Production of 6-({[2-(2-methoxyethoxy)ethyl]amino}methyl)-N-{3-methyl-4-[(6-methylpyridin-3-yl)oxy]phenyl}-8,9-dihydro-7H-pyrimido[4,5-b]azepin-4-amine trihydrochloride

Using 4-({3-methyl-4-[(6-methylpyridin-3-yl)oxy]phenyl}amino)-8,9-dihydro-7H-pyrimido[4,5-b]azepin-6-carbaldehyde (79.4 mg), 2-(2-methoxyethoxy)ethaneamine hydrochloride (65.3 mg), tetrahydrofuran (1.0 mL), N,N-dimethylformamide (1.0 mL) and sodium triacetoxyborohydride (265 mg), a similar reaction as in Example 193 (ii) was carried out to give the title compound (67.9 mg) as a pale-orange powder.

¹H-NMR (DMSO-d₆) δ: 2.20 (3H, s), 2.58 (3H, s), 2.61-2.69 (2H, m), 3.07-3.19 (2H, m), 3.25 (3H, s), 3.44-3.53 (4H, m), 3.56-3.62 (2H, m), 3.72-3.82 (4H, m), 6.95 (1H, s), 7.06 (1H, d, J=8.7 Hz), 7.37 (1H, dd, J=8.7, 2.5 Hz), 7.48 (1H, d, J=2.5 Hz), 7.60 (1H, d, J=8.7 Hz), 7.66-7.75 (1H, m), 8.20 (1H, s), 8.37 (1H, d, J=2.6 Hz), 8.68 (1H, br s), 9.35 (2H, br s), 10.20 (1H, br s).

### Example 227

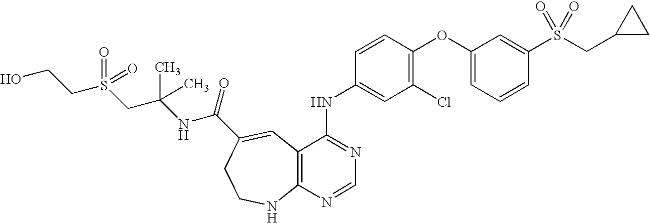

### Production of 4-[(3-chloro-4-{3-[(cyclopropylmethyl)sulfonyl]phenoxy}phenyl)amino]-N-{2-[(2-hydroxyethyl)sulfonyl]-1,1-dimethylethyl}-8,9-dihydro-7H-pyrimido[4,5-b]azepine-6-carboxamide

### (i) Production of 4-[(3-chloro-4-{3-[(cyclopropylmethyl)sulfonyl]phenoxy}phenyl)amino]-8,9-dihydro-7H-pyrimido[4,5-b]azepine-6-carboxylic acid

To a solution of methyl 4-[(3-chloro-4-{3-[(cyclopropylmethyl)sulfonyl]phenoxy}phenyl)amino]-8,9-dihydro-7H-pyrimido[4,5-b]azepine-6-carboxylate (253 mg) in tetrahydrofuran (2.0 mL) and ethanol (2.0 mL) was added 1N aqueous sodium hydroxide solution (2.0 mL) at room temperature. The mixture was stirred at room temperature for 2 days, and 1N hydrochloric acid (2.0 mL) was added thereto. The mixture was concentrated under reduced pressure, and the precipitated crystals were collected by filtration. The crystals were washed with water to give the title compound (232 mg) as pale-yellow crystals.

¹H-NMR (DMSO-d₆) δ: 0.05-0.16 (2H, m), 0.38-0.46 (2H, m), 0.74-0.90 (1H, m), 2.65-2.75 (2H, m), 3.22-3.40 (4H, m), 7.24 (1H, d, J=8.7 Hz), 7.28-7.32 (2H, m), 7.54-7.71 (4H, m), 7.83-7.92 (2H, m), 7.98 (1H, s), 9.39 (1H, s), 12.11-12.25 (1H, m).

### (ii) Production of 4-[(3-chloro-4-{3-[(cyclopropylmethyl)sulfonyl]phenoxy}phenyl)amino]-N-{2-[(2-hydroxyethyl)sulfonyl]-1,1-dimethylethyl}-8,9-dihydro-7H-pyrimido[4,5-b]azepine-6-carboxamide

Using 4-[(3-chloro-4-{3-[(cyclopropylmethyl)sulfonyl]phenoxy}phenyl)amino]-8,9-dihydro-7H-pyrimido[4,5-b]azepine-6-carboxylic acid (100 mg), 2-[(2-amino-2-methylpropyl)sulfonyl]ethanol hydrochloride (83 mg), 1-ethyl-3-(3-dimethylaminopropyl)carbodiimide hydrochloride (73 mg), 1-hydroxybenzotriazole monohydrate (58 mg), triethylamine (0.13 mL) and N,N-dimethylformamide (5.0 mL), a similar reaction as in Example 136 was carried out to give the title compound (78.8 mg) as colorless crystals.

¹H-NMR (CDCl₃) δ: 0.10-0.16 (2H, m), 0.53-0.60 (2H, m), 0.91-1.05 (1H, m), 1.69 (6H, s), 2.68-2.75 (1H, m), 2.85 (2H, t, J=4.5 Hz), 3.00 (2H, d, J=6.9 Hz), 3.22 (2H, t, J=5.1 Hz), 3.52 (2H, q, J=4.5 Hz), 3.69 (2H, s), 4.10 (2H, q, J=5.1 Hz), 5.66-5.72 (1H, m), 6.49 (1H, s), 7.06 (1H, d, J=8.7 Hz), 7.22-7.26 (2H, m), 7.34 (1H, s), 7.36-7.37 (1H, m), 7.47-7.55 (2H, m), 7.59-7.61 (1H, m), 7.87 (1H, d, J=2.7 Hz), 8.12 (1H, s).

### Example 228

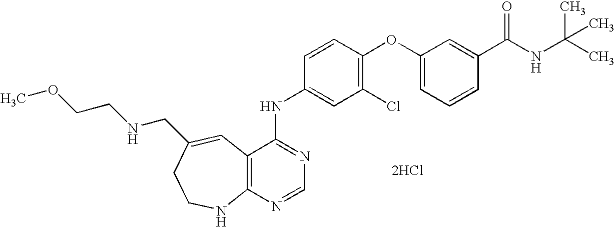

### Production of N-(tert-butyl)-3-{2-chloro-4-[(6-{[(2-methoxyethyl)amino]methyl}-8,9-dihydro-7H-pyrimido[4,5-b]azepin-4-yl)amino]phenoxy}benzamide dihydrochloride

To a solution of N-(tert-butyl)-3-{2-chloro-4-[(6-formyl-8,9-dihydro-7H-pyrimido[4,5-b]azepin-4-yl)amino]phenoxy}benzamide (120 mg) and 2-methoxyethylamine (30.5 mg) in tetrahydrofuran (5.0 mL) was added sodium triacetoxyborohydride (0.31 g) at room temperature. The mixture was stirred at room temperature for 24 hr. Aqueous sodium bicarbonate solution was added to the reaction mixture, and the mixture was extracted with ethyl acetate. The organic layer was washed with water and saturated brine, dried over anhydrous magnesium sulfate and concentrated under reduced pressure. The residue was separated and purified by silica gel column chromatography (eluent, ethyl acetate→methanol:ethyl acetate=10:1) and basic silica gel column chromatography (eluent, ethyl acetate:ethanol:triethylamine=100:20:5) to give colorless amorphous. To the solution of the obtained amorphous in ethanol (5.0 mL) was added 4N hydrochloric acid/ethyl acetate (0.5 mL) at room temperature. The mixture was concentrated under reduced pressure. Ethyl acetate was added to the concentrate and the generated crystals were collected by filtration. The crystals were washed with diisopropyl ether to give the title compound (70.1 mg) as pale-yellow crystals.

¹H-NMR (DMSO-d₆) δ: 1.36 (9H, s), 2.52-2.61 (2H, m), 3.06-3.18 (2H, m), 3.32 (3H, s), 3.38-3.47 (2H, m), 3.65 (2H, t, J=5.0 Hz), 3.68-3.77 (2H, m), 6.85 (1H, br s), 7.05-7.10 (1H, m), 7.17 (1H, d, J=8.7 Hz), 7.30 (1H, br s), 7.40-7.46 (1H, m), 7.52-7.60 (2H, m), 7.81 (1H, s), 7.85 (1H, d, J=2.7 Hz), 8.05-8.21 (2H, m), 9.01-9.17 (2H, m), 9.58-9.78 (1H, m).

### Example 229

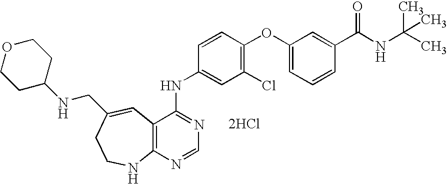

### Production of N-(tert-butyl)-3-[2-chloro-4-({6-[(tetrahydro-2H-pyran-4-ylamino)methyl]-8,9-dihydro-7H-pyrimido[4,5-b]azepin-4-yl}amino)phenoxy]benzamide dihydrochloride

Using N-(tert-butyl)-3-{2-chloro-4-[(6-formyl-8,9-dihydro-7H-pyrimido[4,5-b]azepin-4-yl)amino]phenoxy}benzamide (100 mg), tetrahydro-2H-pyran-4-amine (40 mg), sodium triacetoxyborohydride (0.31 g), tetrahydrofuran (5.0 mL), ethanol (5.0 mL) and 4N hydrochloric acid/ethyl acetate (0.5 mL), a similar reaction as in Example 221 was carried out to give the title compound (73.6 mg) as pale-yellow crystals.

¹H-NMR (DMSO-d₆) δ: 1.36 (9H, s), 1.62-1.79 (2H, m), 2.00-2.09 (2H, m), 2.59-2.65 (2H, m), 3.24-4.00 (9H, m), 6.88 (1H, s), 7.06-7.10 (1H, m), 1.11 (1H, d, J=8.7 Hz), 7.28-7, 33 (1H, m), 7.40-7.45 (1H, m), 7.53-7.63 (2H, m), 7.81 (1H, s), 7.89 (1H, d, J=2.7 Hz), 8.08-8.30 (2H, m), 9.20-9.32 (2H, m), 9.80-9.91 (1H, m).

### Example 230

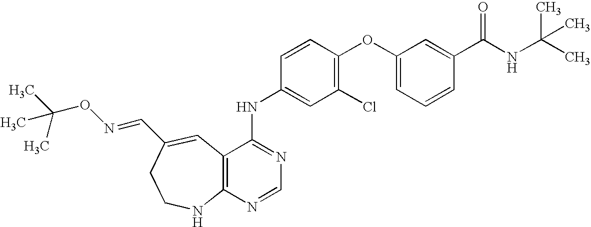

### Production of 3-[4-({6-[(tert-butoxyimino)methyl]-8,9-dihydro-7H-pyrimido[4,5-b]azepin-4-yl}amino)-2-chlorophenoxy]-N-(tert-butyl)benzamide

Using N-(tert-butyl)-3-{2-chloro-4-[(6-formyl-8,9-dihydro-7H-pyrimido[4,5-b]azepin-4-yl)amino]phenoxy}benzamide (100 mg), O-tert-butylhydroxylamine hydrochloride (126 mg), sodium acetate (82 mg) and ethanol (5.0 mL), a similar reaction as in Example 215 was carried out to give the title compound (81.6 mg) as pale-yellow crystals.

¹H-NMR (CDCl₃) δ: 1.32 (9H, s), 1.46 (9H, s), 2.90 (2H, t, J=4.8 Hz), 3.54 (2H, q, J=4.8 Hz), 5.62-5.68 (1H, m), 5.90 (1H, br s), 6.35 (1H, s), 6.49 (1H, s), 7.00 (1H, d, J=9.0 Hz), 7.01-7.05 (1H, m), 7.25-7.41 (4H, m), 7.68 (1H, d, J=2.7 Hz), 7.78 (1H, s), 8.10 (1H, s).

### Example 231

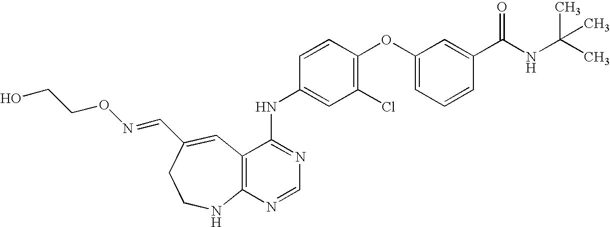

### Production of N-(tert-butyl)-3-{2-chloro-4-[(6-{[(2-hydroxyethoxy)imino]methyl}-8,9-dihydro-7H-pyrimido[4,5-b]azepin-4-yl)amino]phenoxy}benzamide

Using N-(tert-butyl)-3-{2-chloro-4-[(6-formyl-8,9-dihydro-7H-pyrimido[4,5-b]azepin-4-yl)amino]phenoxy}benzamide (100 mg), 2-(aminooxy)ethanol hydrochloride (114 mg), sodium acetate (82 mg) and ethanol (5.0 mL), a similar reaction as in Example 215 was carried out to give the title compound (82.6 mg) as pale-yellow crystals.

¹H-NMR (CDCl₃) δ: 1.46 (9H, s), 2.23-2.32 (1H, m), 2.80-2.88 (2H, m), 3.50-3.56 (2H, m), 3.86-3.95 (2H, m), 4.22-4.25 (2H, m), 5.67-5.73 (1H, m), 5.86-5.95 (1H, m), 6.45 (1H, s), 6.53 (1H, s), 7.00 (1H, d, J=9.0 Hz), 7.02-7.08 (1H, m), 7.25-7.38 (4H, m), 7.67 (1H, d, J=2.7 Hz), 7.86 (1H, s), 8.10 (1H, s).

### Example 232

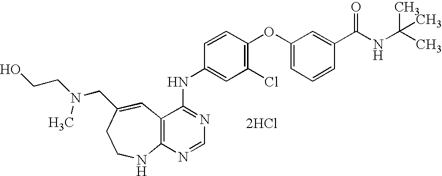

### Production of N-(tert-butyl)-3-{2-chloro-4-[(6-{[(2-hydroxyethyl)(methyl)amino]methyl}-8,9-dihydro-7H-pyrimido[4,5-b]azepin-4-yl)amino]phenoxy}benzamide dihydrochloride

Using N-(tert-butyl)-3-{2-chloro-4-[(6-formyl-8,9-dihydro-7H-pyrimido[4,5-b]azepin-4-yl)amino]phenoxy}benzamide (100 mg) and 2-(methylamino)ethanol (30 mg), sodium triacetoxyborohydride (0.31 g), tetrahydrofuran (5.0 mL), ethanol (5.0 mL) and 4N hydrochloric acid/ethyl acetate (0.5 mL), a similar reaction as in Example 221 was carried out to give the title compound (83.7 mg) as pale-yellow crystals.

¹H-NMR (DMSO-d₆) δ: 1.35 (9H, s), 2.60-2.67 (2H, m), 2.77-2.79 (3H, m), 3.05-3.30 (2H, m), 3.39-3.46 (2H, m), 3.75-4.08 (4H, m), 6.91 (1H, s), 7.06-7.10 (1H, m), 7.17 (1H, d, J=8.7 Hz), 7.26-7.30 (1H, m), 7.40-7.45 (1H, m), 7.50-7.60 (2H, m), 7.78-7.86 (2H, m), 8.09-8.27 (2H, m), 9.56-9.92 (2H, m).

### Example 233

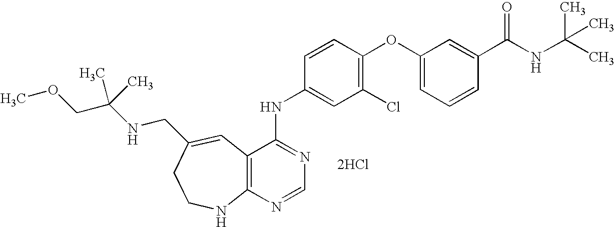

### Production of N-(tert-butyl)-3-{2-chloro-4-[(6-{[(2-methoxy-1,1-dimethylethyl)amino]methyl}-8,9-dihydro-7H-pyrimido[4,5-b]azepin-4-yl)amino]phenoxy}benzamide dihydrochloride

### (i) Production of tert-butyl (2-methoxy-1,1-dimethylethyl)carbamate

To a mixture of tert-butyl (2-hydroxy-1,1-dimethylethyl)carbamate (2.0 g) and tetrabutylammonium hydrogen sulfate (0.36 g) in toluene (20 mL) and 50% aqueous sodium hydroxide solution (4.0 mL) was added methyl iodide (0.98 mL) at room temperature, and the mixture was stirred at room temperature for 24 hr. Methyl iodide (0.98 mL) was added to the reaction mixture, and the mixture was stirred for 3 days. Additional methyl iodide (0.98 mL) was added to the reaction mixture, and the mixture was stirred for 2 days. Water was added to the reaction mixture, and the mixture was extracted with diethyl ether. The organic layer was washed with water and saturated brine, dried over anhydrous magnesium sulfate and concentrated under reduced pressure. The residue was separated and purified by silica gel column chromatography (eluent, hexane:ethyl acetate=9:1→3:1) to give the title compound (1.46 g) as a colorless oil.

¹H-NMR (CDCl₃) δ: 1.29 (6H, s), 1.43 (9H, s), 3.31 (2H, s), 3.37 (3H, s), 4.74 (1H, br s).

### (ii) Production of 1-methoxy-2-methyl-2-propylamine hydrochloride

To a solution of tert-butyl (2-methoxy-1,1-dimethylethyl)carbamate (1.46 g) in ethanol (10 mL) was added 6N hydrochloric acid (3.0 mL) at room temperature. The mixture was stirred at 50° C. for 2 days and concentrated under reduced pressure. Ethanol was added to the residue, and the mixture was concentrated again. Crystallization from ethanol-diisopropyl ether gave the title compound (930 mg) as colorless crystals.

¹H-NMR (DMSO-d₆) δ: 1.21 (6H, s), 3.30-3.74 (5H, m), 7.99 (3H, br s).

### (iii) Production of N-(tert-butyl)-3-{2-chloro-4-[(6-{[(2-methoxy-1,1-dimethylethyl)amino]methyl}-8,9-dihydro-7H-pyrimido[4,5-b]azepin-4-yl)amino]phenoxy}benzamide dihydrochloride

Using N-(tert-butyl)-3-{2-chloro-4-[(6-formyl-8,9-dihydro-7H-pyrimido[4,5-b]azepin-4-yl)amino]phenoxy}benzamide (120 mg), 1-methoxy-2-methyl-2-propylamine hydrochloride (56 mg), triethylamine (40), sodium triacetoxyborohydride (0.31 g), tetrahydrofuran (5.0 mL), N,N-dimethylformamide (1.0 mL), ethanol (5.0 mL) and 4N hydrochloric acid/ethyl acetate (0.5 mL), a similar reaction as in Example 214 was carried out to give the title compound (88 mg) as pale-yellow crystals.

¹H-NMR (DNSO-d₆) δ: 1.36 (15H, s), 2.56-2.64 (2H, m), 3.36 (3H, s), 3.40-3.70 (6H, m), 6.88 (1H, s), 7.05-7.11 (1H, m), 7.17 (1H, d, J=8.7 Hz), 7.30-7.35 (1H, m), 7.40-7.45 (1H, m), 7.53-7.65 (2H, m), 7.81 (1H, s), 7.89 (1H, d, J=2.1 Hz), 8.10-8.30 (2H, m), 8.89-9.00 (2H, m), 9.87-10.02 (1H, m):

### Example 234

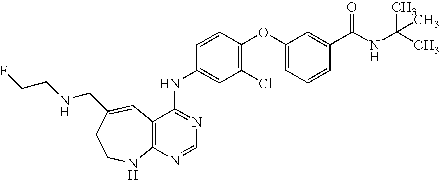

### Production of N-(tert-butyl)-3-{2-chloro-4-[(6-{[(2-fluoroethyl)amino]methyl}-8,9-dihydro-7H-pyrimido[4,5-b]azepin-4-yl)amino]phenoxy}benzamide

To a solution of N-(tert-butyl)-3-{2-chloro-4-[(6-formyl-8,9-dihydro-7H-pyrimido[4,5-b]azepin-4-yl)amino]phenoxy}benzamide (100 mg), 2-fluoroethylamine hydrochloride (40 mg) and triethylamine (56 μL) in tetrahydrofuran (5.0 mL) and N,N-dimethylformamide (1.0 mL) was added sodium triacetoxyborohydride (0.31 g) at room temperature. The mixture was stirred at room temperature for 24 hr. Aqueous sodium bicarbonate solution was added to the reaction mixture, and the mixture was extracted with ethyl acetate. The organic layer was washed with water and saturated brine, dried over anhydrous magnesium sulfate and concentrated under reduced pressure. The residue was separated and purified by silica gel column chromatography (eluent, ethyl acetate) to give the title compound (42 mg) as pale-yellow crystals.

¹H-NMR (CDCl₃) δ: 1.46 (9H, s), 2.55 (2H, t, J=4.8 Hz), 2.95 (2H, dt, J=29.1, 4.8 Hz), 3.41 (2H, s), 3.51 (2H, q, J=4.8 Hz), 4.58 (2H, dt, J=47.4, 4.8 Hz), 5.43-5.50 (1H, m), 5.91 (1H, br s), 6.31 (1H, s), 6.76 (1H, s), 6.99 (1H, d, J=8.4 Hz), 7.01-7.06 (1H, m), 7.30-7.40 (4H, m), 7.71 (1H, d, J=2.4 Hz), 8.08 (1H, s).

### Example 235

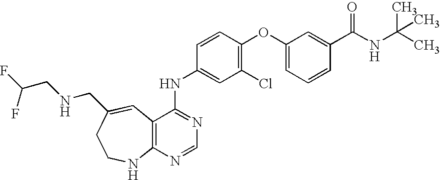

### Production of N-(tert-butyl)-3-{2-chloro-4-[(6-{[(2,2-difluoroethyl)amino]methyl}-8,9-dihydro-7H-pyrimido[4,5-b]azepin-4-yl)amino]phenoxy}benzamide

To a solution of N-(tert-butyl)-3-{2-chloro-4-[(6-formyl-8,9-dihydro-7H-pyrimido[4,5-b]azepin-4-yl)amino]phenoxy}benzamide (100 mg) and 2,2-difluoroethylamine (32 mg) in tetrahydrofuran (5.0 mL) was added sodium triacetoxyborohydride (0.31 g) at room temperature. The mixture was stirred at room temperature for 2 days. Aqueous sodium bicarbonate solution was added to the reaction mixture, and the mixture was extracted with ethyl acetate. The organic layer was washed with water and saturated brine, dried over anhydrous magnesium sulfate and concentrated under reduced pressure. The residue was separated and purified by silica gel column chromatography (eluent, ethyl acetate→ethyl acetate:methanol=19:1) to give the title compound (76 mg) as pale-yellow crystals.

¹H-NMR (CDCl₃) δ: 1.46 (9H, s), 2.56 (2H, t, J=4.8 Hz), 2.99 (2H, td, J=15.3, 4.2 Hz), 3.43 (2H, s), 3.52 (2H, q, J=4.8 Hz), 5.45-5.55 (1H, m), 5.87 (2H, tt, J=56.1, 4.2 Hz), 5.88-5.94 (1H, m), 6.28 (1H, s), 6.61 (1H, s), 6.99 (1H, d, J=9.0 Hz), 7.01-7.06 (1H, m), 7.26-7.41 (4H, m), 7.68 (1H, d, J=2.7 Hz), 8.09 (1H, s).

### Example 236

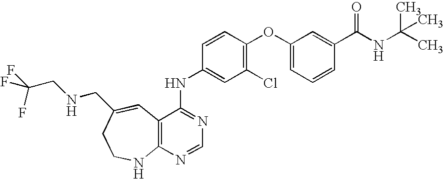

### Production of N-(tert-butyl)-3-{2-chloro-4-[(6-{[(2,2,2-trifluoroethyl)amino]methyl}-8,9-dihydro-7H-pyrimido[4,5-b]azepin-4-yl)amino]phenoxy}benzamide

Using N-(tert-butyl)-3-{2-chloro-4-[(6-formyl-8,9-dihydro-7H-pyrimido[4,5-b]azepin-4-yl)amino]phenoxy}benzamide (100 mg), 2,2,2-trifluoroethylamine (39.6 mg), sodium triacetoxyborohydride (0.31 g) and tetrahydrofuran (5.0 mL), a similar reaction as in Example 235 was carried out to give the title compound (54 mg) as pale-yellow crystals.

¹H-NMR (CDCl₃) δ: 1.46 (9H, s), 2.56 (2H, t, J=4.8 Hz), 3.21 (2H, q, J=9.4 Hz), 3.47 (2H, s), 3.51 (2H, q, J=4.8 Hz), 5.49-5.57 (1H, m), 5.91 (1H, br s), 6.29 (1H, s), 6.58 (1H, s), 6.99 (1H, d, J=9.0 Hz), 7.02-7.06 (1H, m), 7.27-7.40 (4H, m), 7.68 (1H, d, J=2.7 Hz), 8.09 (1H, s).

### Example 237

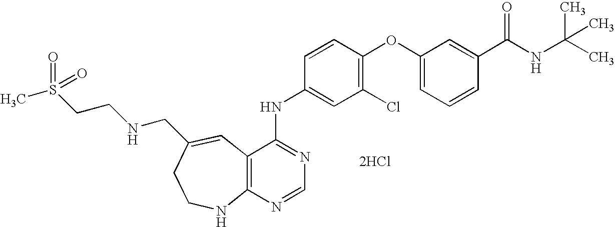

### Production of N-(tert-butyl)-3-(2-chloro-4-{[6-({[2-(methylsulfonyl)ethyl]amino}methyl)-8,9-dihydro-7H-pyrimido[4,5-b]azepin-4-yl]amino}phenoxy)benzamide dihydrochloride

To a solution of N-(tert-butyl)-3-{2-chloro-4-[(6-formyl-8,9-dihydro-7H-pyrimido[4,5-b]azepin-4-yl)amino]phenoxy}benzamide (100 mg) and 2-(methylsulfonyl)ethylamine (49 mg) in tetrahydrofuran (5.0 mL) and N,N-dimethylformamide (1.0 mL) was added sodium triacetoxyborohydride (0.31 g) at room temperature. The mixture was stirred at room temperature for 24 hr. Aqueous sodium bicarbonate solution was added to the reaction mixture, and the mixture was extracted with ethyl acetate. The organic layer was washed with water and saturated brine, dried over anhydrous magnesium sulfate and concentrated under reduced pressure. The residue was separated and purified by silica gel column chromatography (eluent, ethyl acetate→methanol:ethyl acetate=3:7) to give pale-yellow amorphous. 4N Hydrochloric acid/ethyl acetate (0.5 mL) was added to a solution of obtained amorphous in ethanol (5.0 mL) at room temperature. The mixture was concentrated under reduced pressure. Ethyl acetate was added to the residue, and the precipitated crystals were collected by filtration. The crystals were washed with ethyl acetate to give the title compound (93.9 mg) as pale-yellow crystals.

¹H-NMR (DMSO-d₆) δ: 1.36 (9H, s), 2.54-2.65 (2H, m), 3.14 (3H, s), 3.32-3.47 (4H, m), 3.59-3.70 (2H, m), 3.75-3.84 (2H, m), 6.88 (1H, s), 7.07-7.11 (1H, m), 7.18 (1H, d, J=8.7 Hz), 7.28-7.32 (1H, m), 7.40-7.46 (1H, m), 7.54-7.62 (2H, m), 7.81 (1H, s), 7.86 (1H, d, J=2.4 Hz), 8.08-8.25 (2H, m), 9.36-9.49 (2H, m), 9.64-9.85 (1H, m).

### Example 238

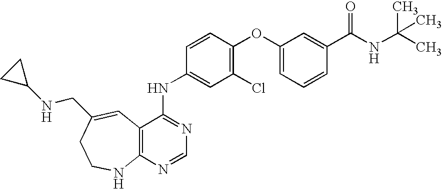

### Production of N-(tert-butyl)-3-[2-chloro-4-({6-[(cyclopropylamino)methyl]-8,9-dihydro-7H-pyrimido[4,5-b]azepin-4-yl}amino)phenoxy]benzamide

Using N-(tert-butyl)-3-{2-chloro-4-[(6-formyl-8,9-dihydro-7H-pyrimido[4,5-b]azepin-4-yl)amino]phenoxy}benzamide (100 mg), cyclopropylamine (22.8 mg), sodium triacetoxyborohydride (0.26 g) and tetrahydrofuran (5.0 mL), a similar reaction as in Example 234 was carried out to give the title compound (63 mg) as pale-yellow crystals.

¹H-NMR (CDCl₃) δ: 0.33-0.41 (2H, m), 0.43-0.51 (2H, m), 1.46 (9H, s), 2.14-2.20 (1H, m), 2.56 (2H, t, J=4.8 Hz), 3.42 (2H, s), 3.51 (2H, q, J=4.8 Hz), 5.43-5.50 (1H, m), 5.90 (1H, br s), 6.21 (1H, s), 6.58 (1H, s), 6.99 (1H, d, J=8.7 Hz), 7.02-7.06 (1H, m), 7.29-7.4.0 (4H, m), 7.68 (1H, d, J=2.7 Hz), 8.08 (1H, s).

### Example 239

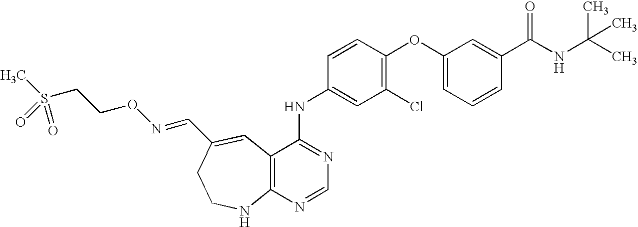

### Production of N-(tert-butyl)-3-(2-chloro-4-{[6-({[2-(methylsulfonyl)ethoxy]imino}methyl)-8,9-dihydro-7H-pyrimido[4,5-b]azepin-4-yl]amino}phenoxy)benzamide

### (i) Production of tert-butyl (2-iodoethoxy)carbamate

To a mixture of tert-butyl (2-hydroxyethoxy)carbamate (1.00 g), triphenylphosphine (1.77 g) and imidazole (0.46 g) in toluene (10 mL) was added iodine (1.57 g) at room temperature. The mixture was stirred at room temperature for 16 hr and at 50° C. for 4 hr. Triphenylphosphine (1.46 g), imidazole (0.38 g) and iodine (1.14 g) were added to the reaction mixture, and the mixture was stirred at 50° C. for 8 hr. Water was added to reaction mixture, and the mixture was extracted with ethyl acetate. The organic layer was washed with aqueous sodium thiosulfate solution and saturated brine, dried over anhydrous magnesium sulfate and concentrated under reduced pressure. Diethyl ether was added to the residue, and the precipitated crystals were removed by filtration. The filtrate was concentrated. Diethyl ether was added to the residue again, and the crystals were removed by filtration. The filtrate was concentrated, and the residue was separated and purified by silica gel column chromatography (eluent, hexane:ethyl acetate=19:1→3:1) to give the title compound (1.15 g) as a colorless oil.

¹H-NMR (CDCl₃) δ: 1.49 (9H, s), 3.33 (2H, t, J=6.9 Hz), 4.09 (2H, t, J=6.9 Hz), 7.16 (1H, s).

### (ii) Production of tert-butyl [2-(methylsulfonyl)ethoxy]carbamate

A mixture of tert-butyl (2-iodoethoxy)carbamate (1.15 g), sodium methanesulfinate (0.49 g) and pyridine (0.39 mL) in N,N-dimethylformamide (10 mL) was stirred at 80° C. for 24 hr. Water was added to reaction mixture, and the mixture was extracted with ethyl acetate. The organic layer was washed with water and saturated brine, dried over anhydrous magnesium sulfate and concentrated under reduced pressure. The residue was separated and purified by silica gel column chromatography (eluent, hexane:ethyl acetate=3:1→1:4) to give the title compound (646.2 mg) as pale-yellow crystals.

¹H-NMR (CDCl₃) δ: 1.48 (9H, s), 3.07 (3H, s), 3.33 (2H, t, J=5.4 Hz), 4.32 (2H, t, J=5.4 Hz), 7.30 (1H, s).

### (iii) Production of 1-(aminooxy)-2-(methylsulfonyl)ethane hydrochloride

Using tert-butyl [2-(methylsulfonyl)ethoxy]carbamate (620 mg), 6N hydrochloric acid (5.0 mL) and ethanol (5.0 mL), a similar reaction as in Example 233 (ii) was carried out to give the title compound (437 mg) as colorless crystals.

¹H-NMR (DMSO-d₆) δ: 3.02 (3H, s), 3.57 (2H, t, J=5.7 Hz), 4.33 (2H, t, J=5.7 Hz).

### (iv) Production of N-(tert-butyl)-3-(2-chloro-4-{[6-({[2-(methylsulfonyl)ethoxy]imino}methyl)-8,9-dihydro-7H-pyrimido[4,5-b]azepin-4-yl]amino}phenoxy)benzamide

Using N-(tert-butyl)-3-{2-chloro-4-[(6-formyl-8,9-dihydro-7H-pyrimido[4,5-b]azepin-4-yl)amino]phenoxy}benzamide (100 mg), 1-(aminooxy)-2-(methylsulfonyl)ethane hydrochloride (70.3 mg), sodium acetate (33 mg) and ethanol (5.0 mL), a similar reaction as in Example 215 was carried out to give the title compound (102 mg) as pale-yellow crystals.

¹H-NMR (CDCl₃) δ: 1.46 (9H, s), 2.84 (2H, t, J=4.8 Hz), 2.96 (3H, s), 3.40 (2H, d, J=5.4 Hz), 3.54 (2H, q, J=4.8 Hz), 4.56 (2H, t, J=5.4 Hz), 5.70-5.80 (1H, m), 5.92 (1H, s), 6.48 (1H, s), 6.58 (1H, s), 7.00 (1H, d, J=8.7 Hz), 7.04-7.08 (1H, m), 7.25-7.37 (4H, m), 7.65 (1H, d, J=2.7 Hz), 7.84 (1H, s), 8.11 (1H, s).

### Example 240

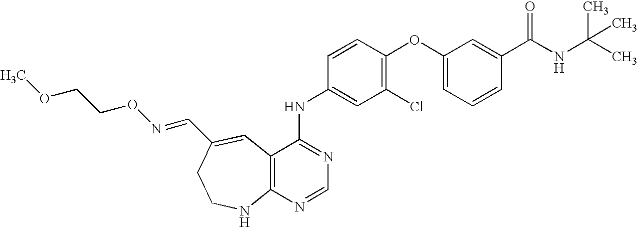

### Production of N-(tert-butyl)-3-{2-chloro-4-[(6-{[(2-methoxyethoxy)imino]methyl}-8,9-dihydro-7H-pyrimido[4,5-b]azepin-4-yl)amino]phenoxy}benzamide

### (i) Production of tert-butyl (2-methoxyethoxy)carbamate

To a solution of tert-butyl N-hydroxycarbamate (2.0 g) in N,N-dimethylformamide (30 mL) was added 60% sodium hydride (dispersion in mineral oil, 0.90 g) at 0° C., and the mixture was stirred at room temperature for 30 min. 2-Bromoethylmethyl ether (1.41 mL) was added to the reaction mixture, and the mixture was stirred at 50° C. for 12 hr. Water was added to reaction mixture, and the mixture was extracted with ethyl acetate. The organic layer was washed with water and saturated brine, dried over anhydrous magnesium sulfate and concentrated under reduced pressure. The residue was separated and purified by silica gel column chromatography (eluent, hexane:ethyl acetate=3:1→3:7) to give the title compound (954 mg) as a colorless oil.

¹H-NMR (CDCl₃) δ: 1.49 (9H, s), 3.39 (3H, s), 3.60-3.63 (2H, m), 4.01-4.04 (2H, m), 7.27 (1H, br s).

### (ii) Production of 1-(aminooxy)-2-methoxyethane hydrochloride

To a solution of tert-butyl (2-methoxyethoxy)carbamate (954 mg) in ethanol (10 mL) was added 6N hydrochloric acid at room temperature. The mixture was stirred at 50° C. for 24 hr and concentrated under reduced pressure. Ethanol was added to the residue, and the mixture was concentrated again to give the title compound (642 mg) as colorless crystals.

¹H-NMR (DMSO-d₆) δ: 3.26 (3H, s), 3.54-3.56 (2H, m), 4.09-4.12 (2H, m), 10.81 (3H, br s).

### (iii) Production of N-(tert-butyl)-3-{2-chloro-4-[(6-{[(2-methoxyethoxy)imino]methyl}-8,9-dihydro-7H-pyrimido[4,5-b]azepin-4-yl)amino]phenoxy}benzamide

Using N-(tert-butyl)-3-{2-chloro-4-[(6-formyl-8,9-dihydro-7H-pyrimido[4,5-b]azepin-4-yl)amino]phenoxy}benzamide (100 mg), 1-(aminooxy)-2-methoxyethane hydrochloride (51 mg), sodium acetate (33 mg) and ethanol (5.0 mL), a similar reaction as in Example 215 was carried out to give the title compound (82 mg) as pale-yellow crystals.

¹H-NMR (CDCl₃) δ: 1.46 (9H, s), 2.86 (2H, t, J=4.8 Hz), 3.41 (3H, s), 3.52 (2H, q, J=4.8 Hz), 3.66 (2H, t, J=4.7 Hz), 4.27 (2H, t, J=4.7 Hz), 5.65-5.71 (1H, m), 5.91 (1H, br s), 6.40 (1H, s), 6.5.6 (1H, s), 6.99 (1H, d, J=8.4 Hz), 7.02-7.06 (1H, m), 7.25-7.41 (4H, m), 7.68 (1H, d, J=2.7 Hz), 7.85 (1H, s), 8.10 (1H, s).

### Example 241

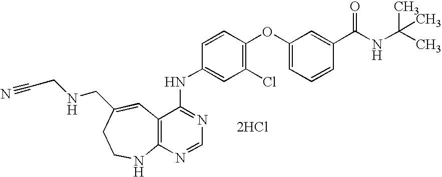

### Production of N-(tert-butyl)-3-{2-chloro-4-[(6-{[(cyanomethyl)amino]methyl}-8,9-dihydro-7H-pyrimido[4,5-b]azepin-4-yl)amino]phenoxy}benzamide dihydrochloride

Using N-(tert-butyl)-3-{2-chloro-4-[(6-formyl-8,9-dihydro-7H-pyrimido[4,5-b]azepin-4-yl)amino]phenoxy}benzamide (100 mg), aminoacetonitrile (34 mg), sodium triacetoxyborohydride (0.34 g), tetrahydrofuran (5.0 mL), ethanol (5.0 mL) and 4N hydrochloric acid/ethyl acetate (0.5 mL), a similar reaction as in Example 221 was carried out to give the title compound (61.2 mg) as pale-yellow crystals.

¹H-NMR (DMSO-d₆) δ: 1.36 (9H, s), 2.54-2.63 (2H, m), 3.30-3.78 (4H, m), 4.21-4.31 (2H, m), 6.80 (1H, br s), 7.05-7.11 (1H, m), 7.18 (1H, d, J=9.0 Hz), 7.28-7.32 (1H, m), 7.40-7.46 (1H, m), 7.51-7.61 (2H, m), 7.79-7.88 (2H, m), 8.05-8.29 (2H, m), 9.50-9.70 (1H, m).

### Example 242

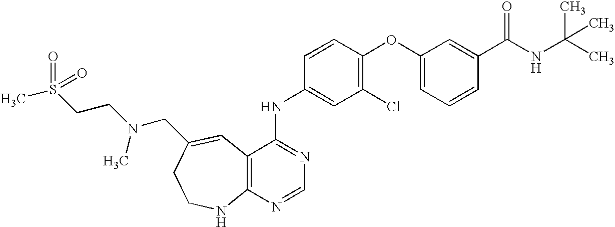

### Production of N-(tert-butyl)-3-(2-chloro-4-{[6-({methyl[2-(methylsulfonyl)ethyl]amino}methyl)-8,9-dihydro-7H-pyrimido[4,5-b]azepin-4-yl]amino}phenoxy)benzamide

Using N-(tert-butyl)-3-{2-chloro-4-[(6-formyl-8,9-dihydro-7H-pyrimido[4,5-b]azepin-4-yl)amino]phenoxy}benzamide (100 mg), N-methyl-2-(methylsulfonyl)ethylamine (80 mg), sodium triacetoxyborohydride (0.25 g) and tetrahydrofuran (5.0 mL), a similar reaction as in Example 235 was carried out to give the title compound (55.3 mg) as pale-yellow crystals.

¹H-NMR (CDCl₃) δ: 1.46 (9H, s), 2.31 (3H, s), 2.53 (2H, t, J=4.8 Hz), 3.01 (3H, s), 3.03 (2H, t, J=6.4 Hz), 3.10 (2H, s), 3.21 (2H, t, J=6.4 Hz), 3.50 (2H, q, J=4.8 Hz), 5.43-5.50 (1H, m), 5.91 (1H, br s), 6.35 (1H, br s), 6.98 (1H, d, J=9.0 Hz), 7.02-7.08 (2H, m), 7.31-7.40 (4H, m), 7.71 (1H, d, J=2.4 Hz), 8.08 (1H, s).

### Example 243

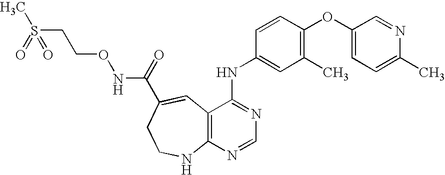

### Production of 4-({3-methyl-4-[(6-methylpyridin-3-yl)oxy]phenyl}amino)-N-[2-(methylsulfonyl)ethoxy]-8,9-dihydro-7H-pyrimido[4,5-b]azepine-6-carboxamide

Using 4-({3-methyl-4-[(6-methylpyridin-3-yl)oxy]phenyl}amino)-8,9-dihydro-7H-pyrimido[4,5-b]azepine-6-carboxylic acid (100 mg), 1-(aminooxy)-2-(methylsulfonyl)ethane hydrochloride (87 mg), 1-ethyl-3-(3-dimethylaminopropyl)carbodiimide hydrochloride (95 mg), 1-hydroxybenzotriazole monohydrate (76 mg), triethylamine (0.18 mL) and N,N-dimethylformamide (5.0 mL), a similar reaction as in Example 136 was carried out to give the title compound (93.9 mg) as colorless crystals.

¹H-NMR (DMSO-d₆) δ: 2.14 (3H, s), 2.43 (3H, s), 2.62-2.68 (2H, m), 3.12 (3H, s), 3.23-3.38 (2H, m), 3.48 (2H, t, J=5.7 Hz), 4.20 (2H, t, J=5.7 Hz), 6.89 (1H, d, J=8.7 Hz), 7.13-7.23 (3H, m), 7.36-7.40 (1H, m), 7.45-7.46 (1H, m), 7.59-7.68 (1H, m), 7.91 (1H, s), 8.14 (1H, d, J=2.4 Hz), 8.92 (1H, s), 11.29-11.41 (1H, m).

### Example 244

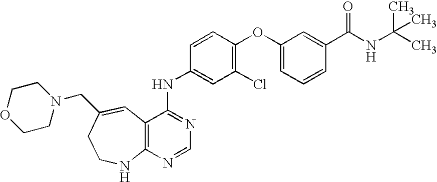

### Production of N-(tert-butyl)-3-(2-chloro-4-{[6-(morpholin-4-ylmethyl)-8,9-dihydro-7H-pyrimido[4,5-b]azepin-4-yl]amino}phenoxy)benzamide

Using N-(tert-butyl)-3-{2-chloro-4-[(6-formyl-8,9-dihydro-7H-pyrimido[4,5-b]azepin-4-yl)amino]phenoxy}benzamide (100 mg), morpholine (35 mg), sodium triacetoxyborohydride (0.25 g) and tetrahydrofuran (5.0 mL), a similar reaction as in Example 235 was carried out to give the title compound (61 mg) as pale-yellow crystals.

¹H-NMR (CDCl₃) δ: 1.46 (9H, s), 2.38-2.46 (4H, m), 2.56-2.64 (2H, m), 3.04 (2H, s), 3.47-3.51 (2H, m), 3.69-3.72 (4H, m), 5.49-5.51 (1H, m), 5.92 (1H, br s), 6.20 (1H, s), 6.57 (1H, s), 7.00 (1H, d, J=9.0 Hz), 7.02-7.06 (1H, m), 7.27-7.39 (4H, m), 7.64 (1H, d, J=2.4 Hz), 8.08 (1H, s).

### Example 245

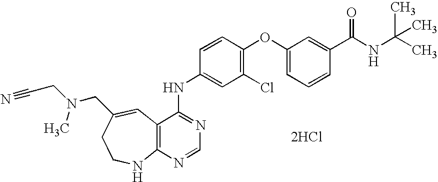

### Production of N-(tert-butyl)-3-{2-chloro-4-[(6-{[(cyanomethyl)(methyl)amino]methyl}-8,9-dihydro-7H-pyrimido[4,5-b]azepin-4-yl)amino]phenoxy}benzamide dihydrochloride

Using N-(tert-butyl)-3-{2-chloro-4-[(6-formyl-8,9-dihydro-7H-pyrimido[4,5-b]azepin-4-yl)amino]phenoxy}benzamide (120 mg), (methylamino)acetonitrile hydrochloride (66 mg), triethylamine (56 μL), sodium triacetoxyborohydride (0.25 g), tetrahydrofuran (5.0 mL), N,N-dimethylformamide (1.0 mL), ethanol (5.0 mL) and 4N hydrochloric acid/ethyl acetate (0.5 mL), a similar reaction as in Example 214 was carried out to give the title compound (25.4 mg) as pale-yellow crystals.

¹H-NMR (DMSO-d₆) δ: 1.36 (9H, s), 2.26 (3H, s), 2.40-2.57 (2H, m), 3.18 (2H, s), 3.36-3.49 (2H, m), 3.77 (2H, s), 6.53 (1H, s), 7.05-7.11 (1H, m), 7.19 (1H, d, J=8.7 Hz), 7.33-7.38 (1H, m), 7.42-7.48 (2H, m), 7.56-7.62 (1H, m), 7.73 (1H, d, J=2.1 Hz), 7.83 (1H, s), 8.17 (1H, s), 9.56-9.75 (1H, m).

### Example 246

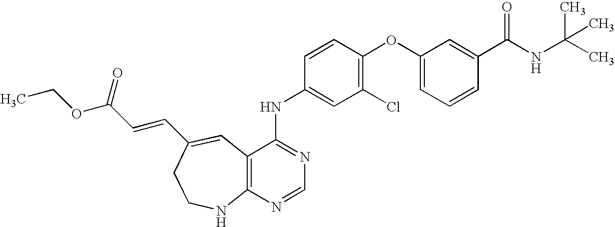

### Production of ethyl (2E)-3-{4-[(4-{3-[(tert-butylamino)carbonyl]phenoxy}-3-chlorophenyl)amino]-8,9-dihydro-7H-pyrimido[4,5-b]azepin-6-yl}acrylate

A solution of N-(tert-butyl)-3-{2-chloro-4-[(6-formyl-8,9-dihydro-7H-pyrimido[4,5-b]azepin-4-yl)amino]phenoxy}benzamide (100 mg) and (carboethoxymethylene)triphenylphosphorane (209 mg) in toluene (5.0 mL) was stirred at 100° C. for 4 hr. Water was added to reaction mixture, and the mixture was extracted with ethyl acetate. The organic layer was washed with water and saturated brine, dried over anhydrous magnesium sulfate and concentrated under reduced pressure. The residue was separated and purified by basic silica gel column chromatography (eluent, hexane:ethyl acetate=1:1→ethyl acetate) to give the title compound (81 mg) as yellow crystals.

¹H-NMR (CDCl₃) δ: 1.32 (3H, t, J=7.2 Hz), 1.46 (9H, s), 2.71 (2H, t, J=4.8 Hz), 3.56 (2H, q, J=4.8 Hz), 4.24 (2H, q, J=7.2 Hz), 5.67-5.74 (1H, m), 5.85 (1H, d, J=15.6 Hz), 5.91 (1H, br s), 6.62-6.68 (2H, m), 7.01 (1H, d, J=9.0 Hz), 7.02-7.06 (1H, m), 7.30-7.42 (4H, m), 7.45 (1H, d, J=15.6 Hz), 7.73 (1H, d, J=2.4 Hz), 8.10 (1H, s).

### Example 247

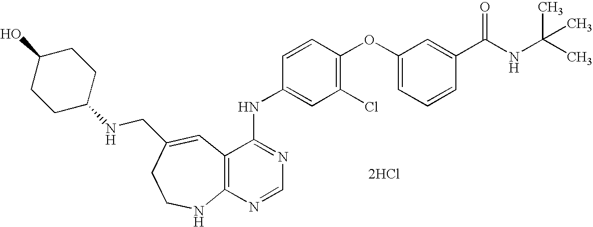

### Production of N-(tert-butyl)-3-{2-chloro-4-[(6-{[(trans-4-hydroxycyclohexyl)amino]methyl}-8,9-dihydro-7H-pyrimido[4,5-b]azepin-4-yl)amino]phenoxy}benzamide dihydrochloride

Using N-(tert-butyl)-3-{2-chloro-4-[(6-formyl-8,9-dihydro-7H-pyrimido[4,5-b]azepin-4-yl)amino]phenoxy}benzamide (100 mg); trans-4-aminocyclohexanol (46 mg), sodium triacetoxyborohydride (0.50 g), tetrahydrofuran (10 mL), ethanol (5.0 mL) and 4N hydrochloric acid/ethyl acetate, (0.5 mL), a similar reaction as in Example 221 was carried out to give the title compound (34.7 mg) as pale-yellow crystals.

¹H-NMR (DMSO-d₆) δ: 1.08-1.25 (2H, m), 1.36 (9H, s), 1.38-1.58 (2H, m), 1.82-1.95 (2H, m), 2.06-2.18 (2H, m), 2.54-2.63 (2H, m), 2.92-3.10 (1H, m), 3.30-4.00 (5H, m), 6.84 (1H, s), 7.07-7.10 (1H, m), 7.17 (1H, d, J=8.7 Hz), 7.31 (1H, br s), 7.43 (1H, t, J=7.8 Hz), 7.56-7.63 (2H, m), 7.81 (1H, s), 7.89 (1H, d, J=2.4 Hz), 8.02-8.27 (2H, m), 8.95-9.09 (2H, m), 9.70-9.93 (1H, m).

### Example 248

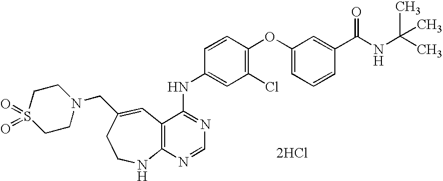

### Production of N-(tert-butyl)-3-[2-chloro-4-({6-[(1,1-dioxidethiomorpholin-4-yl)methyl]-8,9-dihydro-7H-pyrimido[4,5-b]azepin-4-yl}amino)phenoxy]benzamide dihydrochloride

Using N-(tert-butyl)-3-{2-chloro-4-[(6-formyl-8,9-dihydro-7H-pyrimido[4,5-b]azepin-4-yl)amino]phenoxy}benzamide (100 mg), thiomorpholine 1,1-dioxide (54 mg), sodium triacetoxyborohydride (0.25 g), tetrahydrofuran (5.0 mL), ethanol (5.0 mL) and 4N hydrochloric acid/ethyl acetate (0.5 mL), a similar reaction as in Example 221 was carried out to give the title compound (25.5 mg) as pale-yellow crystals.

¹H-NMR (DMSO-d₆) δ: 1.36 (9H, s), 2.53-2.65 (2H, m), 3.13-3.80 (12H, m), 7.05-7.12 (1H, m), 7.18 (1H, d, J=9.0 Hz), 7.27-7.32 (1H, m), 7.41-7.53 (2H, m), 7.56-7.59 (1H, m), 1.11 (1H, br s), 7.81 (1H, s), 8.05-8.29 (2H, m), 9.43-9.65 (1H, m).

### Example 249

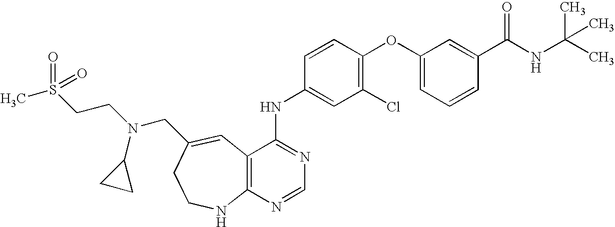

### Production of N-(tert-butyl)-3-(2-chloro-4-{[6-({cyclopropyl[2-(methylsulfonyl)ethyl]amino}methyl)-8,9-dihydro-7H-pyrimido[4,5-b]azepin-4-yl]amino}phenoxy)benzamide

### (i) Production of tert-butyl cyclopropyl[2-(methylsulfonyl)ethyl]carbamate

To a solution of cyclopropylamine (274 mg) in methanol (10 mL) was added dropwise methylvinylsulfone (0.42 mL) at room temperature. The mixture was stirred at room temperature for 16 hr. Di-tert-butyl dicarbonate (1.10 mL) was added to the reaction mixture. The mixture was stirred at room temperature for 4 hr, and concentrated under reduced pressure. The residue was separated and purified by silica gel column chromatography (eluent, hexane:ethyl acetate=9:1→3:7) to give the title compound (1.10 g) as colorless crystals.

¹H-NMR (CDCl₃) δ: 0.62-0.67 (2H, m), 0.75-0.85 (2H, m), 1.47 (9H, s), 2.51-2.59 (1H, m), 2.95 (3H, s), 3.25-3.30 (2H, m), 3.67-3.72 (2H, m).

### (ii) Production of N-[2-(methylsulfonyl)ethyl]cyclopropaneamine hydrochloride

Using tert-butyl cyclopropyl[2-(methylsulfonyl)ethyl]carbamate (1.10 g), 6N hydrochloric acid (4.0 mL) and ethanol (20 mL), a similar reaction as in Example 233 (ii) was carried out to give the title compound (785 mg) as colorless crystals.

¹H-NMR (DMSO-d₆) δ: 0.72-0.79 (2H, m), 0.88-0.93 (2H, m), 2.72-2.79 (1H, m), 3.13 (3H, s), 3.36-3.42 (2H, m), 3.55-3.60 (2H, m), 9.45 (2H, br s).

### (iii) Production of N-(tert-butyl)-3-(2-chloro-4-{[6-({cyclopropyl[2-(methylsulfonyl)ethyl]amino}methyl)-8,9-dihydro-7H-pyrimido[4,5-b]azepin-4-yl]amino}phenoxy)benzamide

To a mixture of N-(tert-butyl)-3-{2-chloro-4-[(6-formyl-8,9-dihydro-7H-pyrimido[4,5-b]azepin-4-yl)amino]phenoxy}benzamide (100 mg), N-[2-(methylsulfonyl)ethyl]cyclopropaneamine hydrochloride (80 mg) and triethylamine (55.8 μL) in tetrahydrofuran (10 mL) and N,N-dimethylformamide (2.0 mL) was added sodium triacetoxyborohydride (0.26 g) at room temperature. The mixture was stirred at room temperature for 24 hr. To the reaction mixture were added N-[2-(methylsulfonyl)ethyl]cyclopropaneamine hydrochloride (160 mg), triethylamine (0.10 mL) and sodium triacetoxyborohydride (0.52 g), and the mixture was stirred at room temperature for 5 days. Aqueous sodium bicarbonate solution was added to the reaction mixture, and the mixture was extracted with ethyl acetate. The organic layer was washed with water and saturated brine, dried over anhydrous magnesium sulfate and concentrated under reduced pressure. The residue was separated and purified by silica gel column chromatography (eluent, ethyl acetate-methyl acetate:methanol=4:1) to give the title compound (45.4 mg) as colorless crystals.

¹H-NMR (CDCl₃) δ: 0.43-0.60 (4H, m), 1.46 (9H, s), 1.85-1.93 (1H, m), 2.48 (2H, t, J=4.8 Hz), 2.97 (3H, s), 3.21-3.34 (6H, m), 3.47 (2H, q, J=4.8 Hz), 5.41-5.48 (1H, m), 5.91 (1H, br s), 6.29 (1H, s), 6.98 (1H, d, J=8.7 Hz), 7.02-7.06 (1H, m), 7.30-7.39 (4H, m), 7.45 (1H, dd, J=8.7, 2.7 Hz), 7.77 (1H, d, J=2.7 Hz), 8.07 (1H, s).

### Example 250

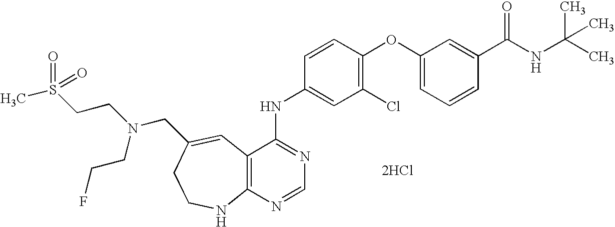

### Production of N-(tert-butyl)-3-(2-chloro-4-{[6-({(2-fluoroethyl) [2-(methylsulfonyl)ethyl]amino}methyl)-8,9-dihydro-7H-pyrimido[4,5-b]azepin-4-yl]amino}phenoxy)benzamide dihydrochloride

### (i) Production of tert-butyl (2-fluoroethyl)[2-(methylsulfonyl)ethyl]carbamate

To a solution of 2-fluoroethylamine hydrochloride (478 mg) and triethylamine (0.67 mL) in methanol (10 mL) was added dropwise methylvinylsulfone (0.42 mL) at room temperature. The mixture was stirred at room temperature for 5 days. Di-tert-butyl dicarbonate (1.10 mL) was added to the reaction mixture. The mixture was stirred at room temperature for 4 hr and concentrated under reduced pressure. Water was added to the residue, and the mixture was extracted with ethyl acetate. The organic layer was washed with water and saturated brine, dried over anhydrous magnesium sulfate and concentrated under reduced pressure. The residue was separated and purified by silica gel column chromatography (eluent, hexane:ethyl acetate=7:3→1:4) to give the title compound (1.13 g) as a colorless oil.

¹H-NMR (CDCl₃) δ: 1.48 (9H, s), 2.94 (3H, s), 3.25-3.40 (2H, m), 3.49-3.65 (2H, m), 3.71-3.76 (2H, m), 4.40-4.67 (2H, m).

### (ii) Production of 2-fluoro-N-[2-(methylsulfonyl)ethyl]ethylamine

Using tert-butyl (2-fluoroethyl) [2-(methylsulfonyl)ethyl]carbamate (1.13 g), 6N hydrochloric acid (5.0 mL) and ethanol (10 mL), a similar reaction as in Example 233 (ii) was carried out to give the title compound (838 mg) as colorless crystals.

¹H-NMR (DMSO-d₆) δ: 3.13 (3H, s), 3.29-3.45 (4H, m), 3.52-3.61 (2H, m), 4.75 (2H, dt, J=47.1, 4.5 Hz), 9.25-9.47 (2H, m).

### (iii) Production of N-(tert-butyl)-3-(2-chloro-4-{[6-({(2-fluoroethyl)[2-(methylsulfonyl)ethyl]amino}methyl)-8,9-dihydro-7H-pyrimido[4,5-b]azepin-4-yl]amino}phenoxy)benzamide dihydrochloride

Using N-(tert-butyl)-3-{2-chloro-4-[(6-formyl-8,9-dihydro-7H-pyrimido[4,5-b]azepin-4-yl)amino]phenoxy}benzamide (100 mg), 2-fluoro-N-[2-(methylsulfonyl)ethyl]ethylamine hydrochloride (123 mg), triethylamine (83 μL), sodium triacetoxyborohydride (0.50 g), tetrahydrofuran (10 mL), N,N-dimethylformamide (2.0 mL), ethanol (5.0 mL) and 4N hydrochloric acid/ethyl acetate (0.5 mL), a similar reaction as in Example 214 was carried out to give the title compound (40.1 mg) as pale-yellow crystals.

¹H-NMR (DMSO-d₆) δ: 1.36 (9H, s), 2.58-2.73 (2H, m), 3.08 (3H, br s), 3.25-5.13 (12H, m), 7.09-7.13 (1H, m), 7.19 (1H, d, J=8.7 Hz), 7.33 (1H, br s), 7.40-7.51 (2H, m), 7.57-7.60 (1H, m), 7.68-7.86 (2H, m), 8.21 (1H, s), 8.39-8.65 (1H, m), 9.71-9.14 (1H, m).

### Example 251

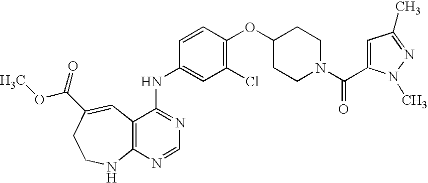

### Production of methyl 4-{[3-chloro-4-({1-[(1,3-dimethyl-1H-pyrazol-5-yl)carbonyl]piperidin-4-yl}oxy)phenyl]amino}-8,9-dihydro-7H-pyrimido[4,5-b]azepine-6-carboxylate

### (i) Production of methyl 4-{[3-chloro-4-(piperidin-4-yloxy)phenyl]amino}-8,9-dihydro-7H-pyrimido[4,5-b]azepine-6-carboxylate

To a suspension of methyl 4-[(4-{[1-(tert-butoxycarbonyl)piperidin-4-yl]oxy}-3-chlorophenyl)amino]-8,9-dihydro-7H-pyrimido[4,5-b]azepine-6-carboxylate (1.0 g) in toluene (20 mL) was added trifluoroacetic acid (8.0 mL) at room temperature. The mixture was stirred at 60° C. for 20 hr and concentrated under reduced pressure. The residue was basified by addition of aqueous sodium bicarbonate solution and extracted with ethyl acetate-tetrahydrofuran (1:1). The organic layer was washed with saturated brine, dried over anhydrous magnesium sulfate and concentrated under reduced pressure. The precipitated crystals were collected by filtration and washed with diisopropyl ether to give the title compound (896 mg) as pale-yellow crystals.

¹H-NMR (CDCl₃) δ: 1.68-1.82 (2H, m), 1.94-2.05 (2H, m), 2.66-2.77 (2H, m), 2.89 (2H, t, J=4.7 Hz), 3.10-3.21 (2H, m), 3.51 (2H, q, J=4.7 Hz), 3.81 (3H, s), 4.30-4.41 (1H, m), 5.72-5.81 (1H, m), 6.76 (1H, s), 6.95 (1H, d, J=8.7 Hz), 7.24-7.27 (1H, m), 7.53 (1H, t, J=2.7 Hz), 7.65 (1H, s), 8.07 (1H, s).

### (ii) Production of methyl 4-{[3-chloro-4-({1-[(1,3-dimethyl-1H-pyrazol-5-yl)carbonyl]piperidin-4-yl}oxy)phenyl]amino}-8,9-dihydro-7H-pyrimido[4,5-b]azepine-6-carboxylate

To a solution of methyl 4-{[3-chloro-4-(piperidin-4-yloxy)phenyl]amino}-8,9-dihydro-7H-pyrimido[4,5-b]azepine-6-carboxylate (100 mg) and pyridine (38 μL) in tetrahydrofuran (5.0 mL) was added dropwise a solution of 1,3-dimethyl-1H-pyrazole-5-carbonylchloride (55 mg) in tetrahydrofuran (3.0 mL) at 0° C. The mixture was stirred at room temperature for 16 hr. Aqueous sodium bicarbonate solution was added to the reaction mixture, and the mixture was extracted with ethyl acetate. The organic layer was washed with water and saturated brine, dried over anhydrous magnesium sulfate and concentrated under reduced pressure. The residue was separated and purified by silica gel column chromatography (eluent, ethyl acetate→ethyl acetate:methanol=4:1) to give the title compound (27 mg) as pale-yellow crystals.

¹H-NMR (CDCl₃) δ: 1.84-2.05 (4H, m), 2.27 (3H, s), 2.89 (2H, t, J=4.8 Hz), 3.52 (2H, q, J=4.8 Hz), 3.57-4.07 (10H, m), 4.56-4.64 (1H, m), 5.75-5.84 (1H, m), 6.09 (1H, s), 6.78 (1H, s), 6.95 (1H, d, J=8.7 Hz), 7.29 (1H, dd, J=8.7, 2.7 Hz), 7.56 (1H, d, J=2.7 Hz), 7.65 (1H, s), 8.07 (1H, s).

### Example 252

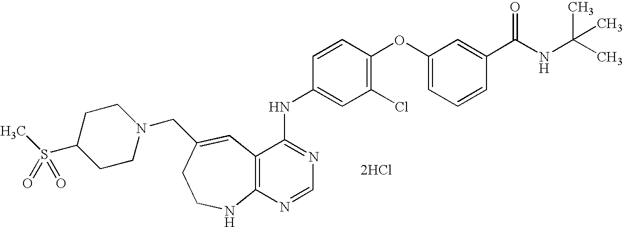

### Production of N-(tert-butyl)-3-{2-chloro-4-[(6-{[4-(methylsulfonyl)piperidin-1-yl]methyl}-8,9-dihydro-7H-pyrimido[4,5-b]azepin-4-yl)amino]phenoxy}benzamide dihydrochloride

### (i) Production of tert-butyl 4-(methylsulfonyl)piperidine-1-carboxylate

To a solution of tert-butyl 4-bromopiperidine-1-carboxylate (2.27 g) in N,N-dimethylformamide (40 mL) was added sodium methanethiolate (0.69 g) at room temperature. The mixture was stirred at 80° C. for 24 hr. Water was added to reaction mixture, and the mixture was extracted with ethyl acetate. The organic layer was washed with water and saturated brine, dried over anhydrous magnesium sulfate and concentrated under reduced pressure. 3-Chloroperbenzoic acid (70%, 4.02 g) was added to a solution of the residue in ethyl acetate (40 mL) at 0° C., and the mixture was stirred at 0° C. for 2 hr. Aqueous sodium thiosulfate solution was added to the reaction mixture, and the mixture was extracted with ethyl acetate. The organic layer was washed with aqueous sodium bicarbonate solution and saturated brine, dried over anhydrous magnesium sulfate and concentrated under reduced pressure. The residue was separated and purified by silica gel column chromatography (eluent, hexane:ethyl acetate=3:1→1:3) to give the title compound (0.69 g) as colorless crystals.

¹H-NMR (CDCl₃) δ: 1.46 (9H, s), 1.65-1.79 (2H, m), 2.07-2.17 (2H, m), 2.66-2.82 (2H, m), 2.84 (3H, s), 2.91-3.02 (1H, m), 4.23-4.40 (2H, m).

### (ii) Production of 4-(methylsulfonyl)piperidine hydrochloride

Using tert-butyl 4-(methylsulfonyl)piperidine-1-carboxylate (0.69 g), 6N hydrochloric acid (5.0 mL) and ethanol (10 mL), a similar reaction as in Example 233 (ii) was carried out to give the title compound (498 mg) as colorless crystals.

¹H-NMR (DMSO-d₆) δ: 1.72-1.91 (2H, m), 2.11-2.22 (2H, m), 2.85-2.95 (2H, m), 2.99 (3H, s), 3.25-3.46 (3H, m), 8.84-9.09 (2H, m).

### (iii) Production of N-(tert-butyl)-3-{2-chloro-4-[(6-{[4-(methylsulfonyl)piperidin-1-yl]methyl}-8,9-dihydro-7H-pyrimido[4,5-b]azepin-4-yl)amino]phenoxy}benzamide dihydrochloride

Using N-(tert-butyl)-3-{2-chloro-4-[(6-formyl-8,9-dihydro-7H-pyrimido[4,5-b]azepin-4-yl)amino]phenoxy}benzamide (120 mg), 4-(methylsulfonyl)piperidine hydrochloride (95.9 mg), triethylamine (67 μL), sodium triacetoxyborohydride (0.31 g), tetrahydrofuran (5.0 mL), N,N-dimethylformamide (1.0 mL), ethanol (5.0 mL) and 4N hydrochloric acid/ethyl acetate (0.5 mL), a similar reaction as in Example 214 was carried out to give the title compound (107 mg) as pale-yellow crystals.

¹H-NMR (DMSO-d₆) δ: 1.35 (9H, s), 2.02-2.38 (4H, m), 2.63-2.73 (2H, m), 2.90-3.07 (4H, m), 3.28-3.93 (8H, m), 6.98 (1H, s), 7.08-7.11 (1H, m), 7.17 (1H, d, J=8.4 Hz), 7.25-7.32 (1H, m), 7.40-7.45 (1H, m), 7.55-7.58 (2H, m), 7.81 (1H, s), 7.84 (1H, d, J=2.4 Hz), 8.15 (1H, s), 8.18-8.39 (1H, m), 9.70-9.94 (1H, m), 10.29-10.48 (1H, m).

### Example 253

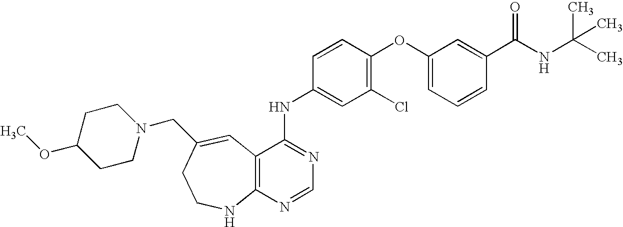

### Production of N-(tert-butyl)-3-[2-chloro-4-({6-[(4-methoxypiperidin-1-yl)methyl]-8,9-dihydro-7H-pyrimido[4,5-b]azepin-4-yl}amino)phenoxy]benzamide

### (i) Production of tert-butyl 4-methoxypiperidine-1-carboxylate

To a mixture of tert-butyl 4-hydroxypiperidine-1-carboxylate (2.0 g), tetra-n-butylammonium hydrogen sulfate (0.34 g), toluene (40 mL) and 50% aqueous sodium hydroxide solution (20 mL) was added methyl iodide (0.93 mL) at room temperature. The mixture was stirred at room temperature for 4 days, and methyl iodide (0.93 mL) was added to the reaction mixture. The mixture was stirred at room temperature for 3 days. Water was added to the mixture, and the mixture was extracted with ethyl acetate. The organic layer was washed with water and saturated brine, dried over anhydrous magnesium sulfate and concentrated under reduced pressure. The residue was separated and purified by silica gel column chromatography (eluent, hexane:ethyl acetate=4:1→1:1) to give the title compound (1.66 g) as a colorless oil.

¹H-NMR (CDCl₃) δ: 1.42-1.55 (11H, m), 1.79-1.89 (2H, m), 3.04-3.13 (2H, m), 3.30-3.40 (4H, m), 3.70-3.81 (2H, m).

### (ii) Production of 4-methoxypiperidine hydrochloride

Using tert-butyl 4-methoxypiperidine-1-carboxylate (1.66 g), 6N hydrochloric acid (10 mL) and ethanol (20 mL), a similar reaction as in Example 233 (ii) was carried out to give the title compound (1.12 g) as colorless crystals.

¹H-NMR (DMSO-d₆) δ: 1.60-1.71 (2H, m), 1.89-1.99 (2H, m), 2.88-2.96 (2H, m), 3.06-3.14 (2H, m), 3.24 (3H, s), 3.39-3.47 (1H, m), 8.71 (2H, br s).

### (iii) Production of N-(tert-butyl)-3-[2-chloro-4-({6-[(4-methoxypiperidin-1-yl)methyl]-8,9-dihydro-7H pyrimido[4,5-b]azepin-4-yl}amino)phenoxy]benzamide

Using N-(tert-butyl)-3-{2-chloro-4-[(6-formyl-8,9-dihydro-7H-pyrimido[4,5-b]azepin-4-yl)amino]phenoxy}benzamide (120 mg), 4-methoxypiperidine hydrochloride (73 mg), triethylamine (67 μL), sodium triacetoxyborohydride (0.31 g), tetrahydrofuran (5.0 mL) and N,N-dimethylformamide (1.0 mL), a similar reaction as in Example 234 was carried out to give the title compound (69.2 mg) as colorless crystals.

¹H-NMR (CDCl₃) δ: 1.46 (9H, s), 1.51-1.71 (2H, m), 1.84-1.96 (2H, m), 2.06-2.19 (2H, m), 2.57 (2H, t, J=4.7 Hz), 2.65-2.79 (2H, m), 3.02 (2H, s), 3.16-3.28 (1H, m), 3.34 (3H, s), 3.48 (2H, q, J=4.7 Hz), 5.52 (1H, t, J=4.2 Hz), 5.91 (1H, br s), 6.21 (1H, s), 6.72 (1H, s), 6.99 (1H, d, J=8.7 Hz), 7.01-7.05 (1H, m), 7.28-7.40 (4H, m), 7.67 (1H, d, J=2.4 Hz), 8.07 (1H, s).

### Example 254

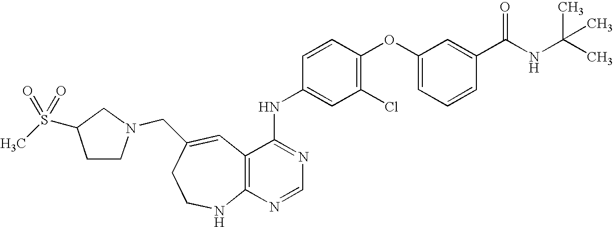

### Production of N-(tert-butyl)-3-{2-chloro-4-[(6-{[3-(methylsulfonyl)pyrrolidin-1-yl]methyl}-8,9-dihydro-7H-pyrimido[4,5-b]azepin-4-yl)amino]phenoxy}benzamide

Using N-(tert-butyl)-3-{2-chloro-4-[(6-formyl-8,9-dihydro-7H-pyrimido[4,5-b]azepin-4-yl)amino]phenoxy}benzamide (120 mg), 3-(methylsulfonyl)pyrrolidine (94 mg), sodium triacetoxyborohydride (0.31 g), tetrahydrofuran (5.0 mL) and N,N-dimethylformamide (2.0 mL), a similar reaction as in Example 235 was carried out to give the title compound (50 mg) as pale-yellow crystals.

¹H-NMR (CDCl₃) δ: 1.46 (9H, s), 2.21-2.37 (2H, m), 2.50-2.64 (3H, m), 2.71-2.78 (1H, m), 2.85-2.97 (4H, m), 3.07-3.12 (1H, m), 3.22-3.33 (2H, m), 3.44-3.64 (3H, m), 5.45-5.54 (1H, m), 5.91 (1H, br s), 6.36 (1H, s), 6.82 (1H, s), 6.98 (1H, t, J=8.7 Hz), 7.02-7.06 (1H, m), 7.31-7.40 (4H, m), 7.69 (1H, d, J=2.7 Hz), 8.07 (1H, s).

### Example 255

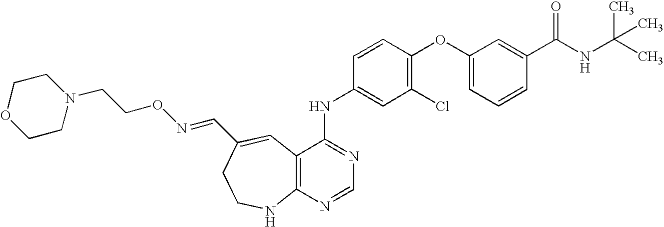

### Production of N-(tert-butyl)-3-{2-chloro-4-[(6-{[(2-morpholin-4-ylethoxy)imino]methyl}-8,9-dihydro-7H-pyrimido[4,5-b]azepin-4-yl)amino]phenoxy}benzamide

### (i) Production of tert-butyl (2-morpholin-4-ylethoxy)carbamate

To a solution of tert-butyl (2-hydroxyethoxy)carbamate (1.0 g) and triethylamine (1.57 mL) in tetrahydrofuran (30 mL) was added methanesulfonyl chloride (0.66 mL) at 0° C. The mixture was stirred at 0° C. for 2 hr. Aqueous sodium bicarbonate solution was added to the reaction mixture, and the mixture was extracted with ethyl acetate. The organic layer was washed with water and saturated brine, dried over anhydrous magnesium sulfate and concentrated under reduced pressure. Morpholine (0.99 mL) and potassium carbonate (1.56 g) were added to a solution of the residue (1.66 g) in N,N-dimethylformamide (30 mL) at room temperature. The mixture was stirred at 90° C. for 2 days. Water was added to reaction mixture, and the mixture was extracted with ethyl acetate. The organic layer was washed with water and saturated brine, dried over anhydrous magnesium sulfate and concentrated under reduced pressure. The residue was separated and purified by silica gel column chromatography (eluent, hexane:ethyl acetate=1:1→ethyl acetate) to give the title compound (1.01 g) as a yellow oil.

¹H-NMR (CDCl₃) δ: 1.48 (9H, s), 2.45-2.55 (2H, m), 2.66 (2H, t, J=5.4 Hz), 3.72-3↓(4H, m), 3.99 (2H, t, J=5.4 Hz), 7.69 (1H, m).

### (ii) Production of 4-[2-(aminooxy)ethyl]morpholine dihydrochloride

Using tert-butyl (2-morpholin-4-ylethoxy)carbamate (1.01 g), 6N hydrochloric acid (10 mL) and ethanol (20 mL), a similar reaction as in Example 233 (ii) was carried out to give the title compound (654 mg) as colorless crystals.

¹H-NMR (DMSO-d₆) δ: 3.21-3.30 (4H, m), 3.43 (2H, t, J=4.8 Hz), 3.87 (4H, t, J=4.7 Hz), 4.30-4.40 (2H, m), 10.29-11.03 (3H, m).

### (iii) Production of N-(tert-butyl)-3-{2-chloro-4-[(6-{[(2-morpholin-4-ylethoxy)imino]methyl}-8,9-dihydro-7H-pyrimido[4,5-b]azepin-4-yl)amino]phenoxy}benzamide

Using N-(tert-butyl)-3-{2-chloro-4-[(6-formyl-8,9-dihydro-7H-pyrimido[4,5-b]azepin-4-yl)amino]phenoxy}benzamide (120 mg), 4-[2-(aminooxy)ethyl]morpholine dihydrochloride (105 mg), sodium acetate (39 mg) and ethanol (5.0 mL), a similar reaction as in Example 215 was carried out to give the title compound (97 mg) as pale-yellow crystals.

¹H-NMR (CDCl₃) δ: 1.46 (9H, s), 2.53 (4H, t, J=4.5 Hz), 2.70 (2H, t, J=5.7 Hz), 2.86 (2H, t, J=5.7 Hz), 3.48-3.58 (2H, m), 3.73 (4H, t, J=4.5 Hz), 4.26 (2H, t, J=5.7 Hz), 5.66-5.74 (1H, m), 5.89-5.95 (1H, m), 6.41 (1H, s), 6.54 (1H, s), 7.00 (1H, d, J=9.0 Hz), 7.03-7.06 (1H, m), 7.26-7.40 (4H, m), 7.67 (1H, d, J=2.4 Hz), 7.83 (1H, s), 8.10 (1H, s).

### Example 256

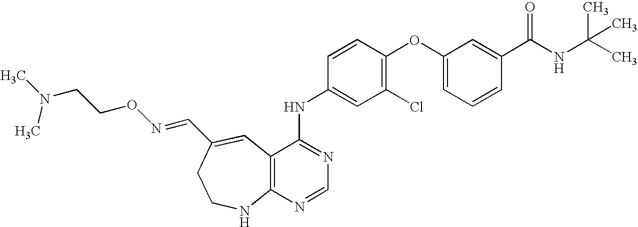

### Production of N-(tert-butyl)-3-(2-chloro-4-{[6-({[2-(dimethylamino)ethoxy]imino}methyl)-8,9-dihydro-7H-pyrimido[4,5-b]azepin-4-yl]amino}phenoxy)benzamide

### (i) Production of tert-butyl [2-(dimethylamino)ethoxy]carbamate

Using tert-butyl (2-hydroxyethoxy)carbamate (1.0 g), triethylamine (1.57 mL), methanesulfonyl chloride (0.66 mL), tetrahydrofuran (30 mL), dimethylamine hydrochloride (1.40 g), potassium carbonate (2.88 g) and N,N-dimethylformamide (20 mL), a similar reaction as in Example 255 (i) was carried out to give the title compound (580 mg) as a colorless oil.

¹H-NMR (CDCl₃) δ: 1.48 (9H, s), 2.28 (6H, s), 2.57 (2H, t, J=5.4 Hz), 3.95 (2H, t, J=5.4 Hz), 7.66 (1H, br s).

### (ii) Production of 2-(aminooxy)-N,N-dimethylethylamine dihydrochloride

Using tert-butyl [2-(dimethylamino)ethoxy]carbamate (580 mg), 6N hydrochloric acid (5.0 mL) and ethanol (10 mL), a similar reaction as in Example 233 (ii) was carried out to give the title compound (358 mg) as colorless crystals.

¹H-NMR (DMSO-d₆) δ: 2.79 (6H, s), 3.40 (2H, t, J=4.7 Hz), 4.36 (2H, t, J=4.7 Hz), 10.53-11.10 (3H, m).

### (iii) Production of N-(tert-butyl)-3-(2-chloro-4-{[6-({[2-(dimethylamino)ethoxy]imino}methyl)-8,9-dihydro-7H-pyrimido[4,5-b]azepin-4-yl]amino}phenoxy)benzamide

Using N-(tert-butyl)-3-{2-chloro-4-[(6-formyl-8,9-dihydro-7H-pyrimido[4,5-b]azepin-4-yl)amino]phenoxy}benzamide (120 mg), 2-(aminooxy)-N,N-dimethylethylamine dihydrochloride (85 mg), sodium acetate (39 mg) and ethanol (5.0 mL), a similar reaction as in Example 215 was carried out to give the title compound (47 mg) as pale-yellow crystals.

¹H-NMR (CDCl₃) δ: 1.46 (9H, s), 2.30 (6H, s), 2.62 (2H, t, J=5.7 Hz), 2.86 (2H, t, J=4.8 Hz), 3.53 (2H, q, J=4.8 Hz), 4.21 (2H, t, J=5.7 Hz), 5.65-5.74 (1H, m), 5.91 (1H, br s), 6.39 (1H, s), 6.58 (1H, s), 6.99 (1H, d, J=8.7 Hz), 7.01-7.06 (1H, m), 7.23-7.41 (4H, m), 7.67 (1H, d, J=2.4 Hz), 7.84 (1H, s), 8.09 (1H, s).

### Formulation Example 1

### Amount Per Tablet

| | | |
|---|---|---|
| (1) | Compound obtained in Example 119 | 10.0 mg |
| (2) | Lactose | 60.0 mg |
| (3) | Corn starch | 35.0 mg |
| (4) | Gelatin | 3.0 mg |
| (5) | Magnesium stearate | 2.0 mg |

A mixture of 10.0 mg of the compound obtained in Example 119, 60.0 mg of lactose and 35.0 mg of corn starch is granulated through a 1 mm-mesh sieve using 0.03 ml of a 10% by weight aqueous solution of gelatin (3.0 mg of gelatin), after which the granules are dried at 40° C. and filtered again. The obtained granules are mixed with 2.0 mg of magnesium stearate and compressed. The obtained core tablets are coated with a sugar coat comprising a suspension of sucrose, titanium dioxide, talc and gum arabic and polished with beeswax to yield sugar-coated tablets.

### Formulation Example 2

### Dose Per Tablet

| | | |
|---|---|---|
| (1) | Compound obtained in Example 119 | 10.0 mg |
| (2) | Lactose | 70.0 mg |
| (3) | Corn starch | 50.0 mg |
| (4) | Soluble starch | 7.0 mg |
| (5) | Magnesium stearate | 3.0 mg |

10.0 mg of the compound obtained in Example 119 and 3.0 mg of magnesium stearate are granulated using 0.07 ml of an aqueous solution of soluble starch (7.0 mg of soluble starch), after which these granules are dried and mixed with 70.0 mg of lactose and 50.0 mg of corn starch. This mixture is compressed to yield tablets.

### Experimental Example 1A

### Cloning of Human HER2 Gene and Preparation of Recombinant Baculovirus

Human HER2 gene was cloned by RT-PCR using total RNA prepared from MCF7 cells as a template. The primer used for RT-PCR was prepared from base sequence (Genbank Accession No. M11730) information of HER2 gene, adding base sequence encoding DYKDDDD peptide and a restriction enzyme recognition sequence to a nucleotide sequence (2176-3918 of Genbank Accession No. M11730 as a base, and corresponding to 676-1255 amino acid of Genbank Accession No. NP_{—}004439 as a protein) encoding the HER2 intracellular domain region, so that a DYKDDDD peptide tag would be attached to the N-terminus of the protein. The base sequence of the primer is shown in below.

(SEQ ID NO: 1)

HER2-U:
5′-
AATTAAGTCGACATGGACTACAAAGACGATGACGACAAGCGACGGCAGCA
GAAGATCCGGAAGTAC-3′
and

(SEQ ID NO: 2)

HER2-L:
5′-AATTAAGCATGCTCACACTGGCACGTCCAGACCCAGGTACTC-3′

The RT reaction was performed using Superscript First-Strand Synthesis System for RT-PCR (Invitrogen) and the PCR reaction was performed using KOD-plus kit (TOYOBO). The obtained PCR product was electrophoresed on agarose gel (1%), and the DNA fragment amplified by PCR was recovered from the gel, and then digested with restriction enzymes Sal I and Sph I. The DNA treated with the restriction enzymes was electrophoresed on agarose gel (1%), and the obtained DNA fragment was recovered and ligated to plasmid pFASTBAC1 (Invitrogen) digested with restriction enzymes Sal I and Sph I to give expression plasmid pFB-HER2. The base sequence of the inserted fragment was confirmed and found to be identical with the base sequence of HER2 intracellular domain (2176-3918 of Genbank Accession M11730). Furthermore, using BAC-TO-BAC Baculovirus Expression System (Invitrogen), recombinant baculovirus BAC-HER2 was prepared.

### Experimental Example 1B

### Preparation of HER2 Intracellular Domain Protein

SF-21 cells were sown at 1×10⁶ cells/mL to 1 L of Sf-900II SFM medium (Invitrogen) containing 10% fetal calf serum (trace), 50 mg/L gentamicin (Invitrogen) and 0.1% Pluronic F-68 (Invitrogen), and cultured with shaking in 2 L volume Erlenmeyer flask at 27° C., 100 rpm. After culturing for 24 hr, recombinant baculovirus BAC-HER2 (13.4 mL) was added thereto, and the cells were further cultured for 3 days. The culture medium was centrifuged at 2,000 rpm for 5 min to give virus-infected cells. The infected cells were washed with phosphate buffered saline (Invitrogen), centrifuged under the same conditions, and preserved at −80° C. The cryopreserved cells were thawed in ice and suspended in buffer A (50 mM Tris buffer (30 mL, pH 7.4) containing 20% glycerol, 0.15 M NaCl) supplemented with Complete Protease Inhibitor (Boehringer). The cells were ruptured 3 times with a Polytron homogenizer (Kinematica) at 20,000 rpm for 30 sec. The disrupt was clarified by centrifugation at 40,000 rpm for 30 min and filtered with a 0.45 μm filter. The filtrate was passed through a column packed with Anti-FLAG M2 Affinity Gel (4 mL, Sigma Aldrich CO.) at a flow rate of about 0.5 mL/min. The column was washed with buffer A, and eluted with buffer A containing 100 μg/mL of FLAG peptide. The eluate was concentrated with Vivaspin 20 (Vivascience) having a molecular weight cut off of 30K. The concentrate was purified by gel filtration using Hi-Load 16/60 Superdex 200pg (GE Healthcare Bioscience) equilibrated with buffer A. The fractions containing HER2 intracellular domain were collected and cryopreserved at −80° C.

### Experimental Example 1C

### Measurement of HER2 Kinase Inhibitory Activity

HER2 kinase reaction was carried out using 96-well plate. As a buffer for Kinase reaction, a buffer of a composition of 50 mM Tris-HCl (pH 7.5), 5 mM MnCl₂, 2 mM dithiothreitol and 0.01% Tween-20 was used. The compound of the present invention was dissolved in dimethyl sulfoxide (DMSO), and diluted with a buffer for kinase reaction so that the DMSO concentration would become 0.1% during kinase reaction. The compound solution (10 μL) was mixed with a buffer for kinase reaction (20 μL) containing the HER2 intracellular domain (0.625 μg/mL) obtained in Experimental Example 1B and a polypeptide substrate poly-Glu:Tyr (4:1) (12.5 μg/mL, Sigma Ltd.) and the mixture was stood for 5 min at room temperature. Then, a buffer for kinase reaction (20 μL) containing 125 μM ATP and 45 μCi/mL [γ-³²P]ATP was added thereto, and the kinase reaction was started at the final reaction mixture amount of 50 μL. After kinase reaction at room temperature for 10 min, a 20% TCA solution (50 μL) was added to quench the kinase reaction. The mixture after reaction completion was stood at room temperature for 30 min, acid insoluble fractions in the mixture after reaction completion were collected iii a 96-well GF/C filter plate (PerkinElmer) using a cell harvester (PerkinElmer), and a filter containing the acid-insoluble fractions was washed with a 3% phosphoric acid solution. The filter plate after washing was dried at 45° C. for 60 min, 25 μL of microscinti 0 (PerkinElmer) was added, and the radioactivity was determined using a Top Count (PerkinElmer). The HER2 kinase inhibitory rate (%) of the test compound was calculated by the following formula:

Inhibitory rate (%)=(1−(count of test compound−blank)÷(control−blank))×100

The count of the solution reacted without addition of the compound was used as a “control”, and the count of the solution without the compound and HER2 intracellular domain was used as a “blank”. The results of the inhibitory rate of the compounds are shown in Table 1.

From the foregoing, it has been shown that the compound of the present invention strongly inhibits the activity of HER2 kinase.

TABLE 1

| Example No.(Compound no.) | Inhibitory rate (%) at 1.0 μM |
|---|---|
| 135 | 97 |
| 122 | 96 |
| 146 | 92 |
| 172 | 95 |
| 214 | 99 |
| 220 | 96 |
| 227 | 93 |
| 231 | 97 |
| 234 | 98 |
| 242 | 99 |
| 246 | 98 |

### Experimental Example 2A

### Cloning of Human EGFR Gene and Preparation of Recombinant Baculovirus

Human EGFR gene was cloned by RT-PCR using total RNA prepared from A431 cell as a template. The primer used for RT-PCR was prepared from base sequence (Genbank Accession No. X00588) information of EGFR gene, adding DYKDDDD base sequence encoding the peptide and a restriction enzyme recognition sequence to a base sequence (2191-3819 of Genbank Accession No. X00588 as a base, and corresponding to 669-1210 amino acid of Genbank Accession No. NP_{—}005219 as a protein) encoding the EGFR intracellular domain, so that a DYKDDDD peptide tag would be attached to the N-terminus of the protein. The base sequence of the primer is shown in below.

(SEQ ID NO: 3)

EGFR-U:
5′-
AATTAAGTCGACATGGACTACAAAGACGATGACGACCGAAGGCGCCACAT
CGTTCGGAAGCGCACG-3′
and

(SEQ ID NO: 4)

EGFR-L:
5′-AATTAAGCATGCTCATGCTCCAATAAATTCACTGCTTTGTGG-3′

The RT reaction was performed using Superscript First-Strand Synthesis System for RT-PCR (Invitrogen), and PCR reaction was performed using KOD-plus kit (TOYOBO). The obtained PCR product was electrophoresed on agarose gel (1%), and the DNA fragment amplified by PCR was recovered from the gel, and then digested with restriction enzymes Sal I and Sph I. The DNA treated with the restriction enzymes was electrophoresed on agarose gel (1%), and the obtained DNA fragment was recovered and ligated to plasmid pFASTBAC1 (Invitrogen) digested with restriction enzymes Sal I and Sph I to give expression plasmid pFB-EGFR. The base sequence of the inserted fragment was confirmed and found to be identical with the base sequence of the EGFR intracellular domain (2191-3819 of Genbank Accession X00588). Furthermore, virus stock BAG-EGFR of recombinant Baculovirus was prepared using BAC-TO-BAC Baculovirus Expression System (Invitrogen).

### Experimental Example 2B

### Preparation of Human EGFR Intracellular Domain Protein

SF-21 cells were sown at 1×10⁶ cells/mL to 1 L of Sf-900II SFM medium (Invitrogen) containing 10% fetal calf serum (trace), 50 mg/L Gentamicin (Invitrogen) and 0.1% Pluronic F-68 (Invitrogen), and cultured with shaking in a 2 L Erlenmeyer flask at 27° C., 100 rpm. After culture for 24 hr, recombinant Baculovirus BAC-EGFR (13.4 mL) was added thereto, and the cells were further cultured for 3 days. The culture medium was centrifuged at 2,000 rpm for 5 min to give virus infected cells. The infected cells were washed with phosphate buffered saline (Invitrogen), centrifuged under the same conditions, and preserved at −80° C. The cryopreserved cells were thawed in ice and suspended in 30 mL of buffer A containing Complete Protease Inhibitor (Boehringer) (50 mM tris buffer (pH 7.4) containing 20% Glycerol, 0.15 M NaCl). The cells were disrupted 3 times at 20,000 rpm for 30 sec using a polytron homogenizer (Kinematica). The disrupt was clarified by centrifugation at 40,000 rpm for 30 min and filtered through a 0.45 μm filter. The filtrate was passed through a column packed with 4 mL of Anti-FLAG M2 Affinity Gel (Sigma Aldrich Co.) at a flow rate of about 0.5 mL/min. The column was washed with buffer A, and eluted with buffer A containing 100 μg/mL of FLAG peptide. The eluate was concentrated with Vivaspin 20 having a molecular weight cut off of 30 K (Vivascience). The concentrate was purified by gel filtration using Hi-Load 16/60 Superdex 200 pg (GE Healthcare Bioscience) equilibrated with buffer A. The fractions containing EGFR intracellular domain were collected and cryopreserved at −80° C.

### Experimental Example 2C

### Assay of EGFR Kinase Inhibitory Activity

EGFR kinase reaction was carried out using a 96-well plate. As a buffer for Kinase reaction, a buffer of a composition of 50 mM Tris-HCl (pH 7.5), 5 mM MnCl₂, 2 mM dithiothreitol and 0.01% Tween-20 was used. The compound of the present invention was dissolved in dimethyl sulfoxide (DMSO), and diluted with a buffer for kinase reaction so that the DMSO concentration would become 0.1% during kinase reaction. The compound solution (10 μL) was mixed with a buffer for kinase reaction (20 μL) containing the EGFR intracellular domain (0.625 μg/mL) obtained in Experimental Example 2B and a polypeptide substrate poly-Glu:Tyr (4:1) (12.5 μg/mL, Sigma Ltd.) and the mixture was stood for 5 min at room temperature. Then, a buffer for kinase reaction (20 μL) containing 125 ATP and 45 μCi/mL [γ-³²P]ATP was added thereto, and the kinase reaction was started at the final reaction mixture amount of 50 μL. After kinase reaction at room temperature for 10 min, a 20% TCA solution (50 μL) was added to quench the kinase reaction. The mixture after reaction completion was stood at room temperature for 30 min, acid insoluble fractions in the mixture after reaction completion were collected in a 96-well GF/C filter plate (PerkinElmer) using a cell harvester (PerkinElmer), and a filter containing the acid-insoluble fractions was washed with a 3% phosphoric acid solution. The filter plats after washing was dried at 45° C. for 60 min, 25 μL of microscinti 0 (PerkinElmer) was added, and the radioactivity was determined using a Top Count (PerkinElmer). The EGFR kinase inhibitory rate (%) of the test compound was calculated by the following formula:

Inhibitory rate (%)=(1−(count of test compound−blank)÷(control−blank))×100

The count of the solution reacted without addition of the compound was used as a “control”, and the count of the solution without the compound and EGFR intracellular domain was used as a “blank”. The results of the inhibitory rate of the compounds are shown in Table 2.

From the foregoing, it has been shown that the compound of the present invention strongly inhibits the activity of EGFR kinase.

TABLE 2
EGFR inhibitory activity

| Example No.(compound No.) | Inhibitory rate (%) at 1.0 μM |
|---|---|
| 115 | 95 |
| 119 | 96 |
| 136 | 97 |
| 143 | 97 |
| 147 | 95 |

### Experimental Example 3

### Inhibitory Action on Breast Cancer Cell BT-474 Proliferation In Vitro

A suspension of human breast cancer cell BT-474 (100 μl (6,000 cells)) were plated to 96-well microplate and cultured in an incubator (37° C., 5% carbon dioxide). On the following day, 100 μl of a solution of each test compound previously diluted serially in 2-fold, was added to give indicated dose, and the cells were cultured for 5 days. The culture medium containing the test compound was removed, and the cells were washed, and fixed with 50% trichloroacetic acid, and 0.4% w/v) SRB solution (dissolved in 1% acetic acid) was added to stain the cell protein (Skehan et al., Journal of the National Cancer Institute, Vol. 82, pp. 1107-1112, 1990). After washing with a 1% acetic acid solution, 100 μl of Tris solution (10 mM) was added to extract the pigment and the absorbance was measured at a wavelength of 550 nm to quantify the amount of cells as protein content. Taking as 100% the protein content for the control group, which received no test compound solution, the ratio of the residual protein content for each treatment group was determined, and the compound concentration required to achieve 50% suppression of the residual cell content relative to the control (IC₅₀ value) was calculated. The results are shown in Table 3.

TABLE 3

| Example No. (compound No.) | IC₅₀ (nM) |
|---|---|
| 119 | <100 |
| 122 | <100 |
| 143 | <100 |

### INDUSTRIAL APPLICABILITY

According to the present invention, pyrimidoazepin compound and pyrimidoazocine compound, production methods thereof and use thereof are provided. These condensed pyrimidine compounds have a superior tyrosine kinase inhibitory action, are highly safe, and are sufficiently satisfactory as pharmaceutical products.

This application is based on U.S. Provisional application No. 60/775,777 filed in the United States, the contents of which are hereby incorporated by reference. All of the references cited herein, including patents, patent applications and publications, are hereby incorporated in their entireties by reference.

1. A compound represented by the formula: 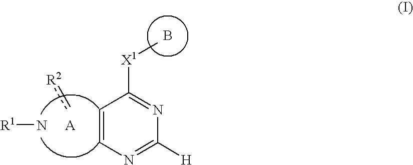
wherein
ring a is an optionally further substituted nitrogen-containing 7-membered or 8-membered ring,
ring b is an optionally substituted aryl group or an optionally substituted heteroaryl group,
X¹ is a group represented by —NR³—Y¹—, —O—, —S—, —SO—, —SO₂— or —CHR³—
wherein
R³ is a hydrogen atom or an optionally substituted aliphatic hydrocarbon group, or
R³ is optionally bonded to the carbon atom on ring b to form an optionally substituted ring structure, and
Y¹ is a bond or an optionally substituted C₁₋₄ alkylene,
R¹ is a hydrogen atom, or an optionally substituted group bonded via a carbon atom or a sulfur atom,
formula [symbol] is a single bond or a double bond, and
R² is a hydrogen atom, or an optionally substituted group bonded via a carbon atom, a nitrogen atom, an oxygen atom or a sulfur atom when [symbol] R² is —R², and an oxo group, an optionally substituted alkylidene group or an optionally substituted imino group when [symbol] R² is ═R², or
R¹ and R², or R² and R³ are optionally bonded to each other to form an optionally substituted ring structure,
or a salt thereof.

2. The compound of claim 1, which is a compound represented by the formula: 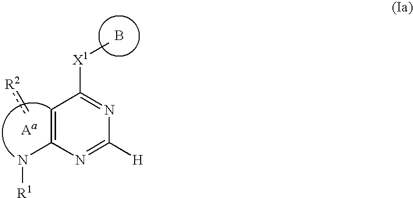
wherein ring A^{a} is an optionally further substituted nitrogen-containing 7-membered or 8-membered ring, and other symbols are as defined in claim 1,
or a salt thereof.

3. The compound of claim 1, which is a compound represented by the formula: 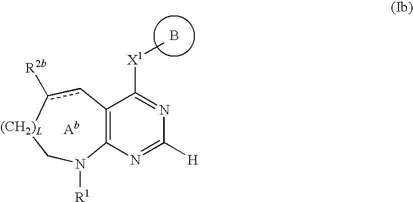
wherein
ring A^{b} is an optionally further substituted nitrogen-containing 7-membered or 8-membered ring,
L is 1 or 2,
formula [symbol] is a single bond or a double bond,
R^{2b} is a hydrogen atom, or an optionally substituted group bonded via a carbon atom, a nitrogen atom, an oxygen atom or a sulfur atom, and
other symbols are as defined in claim 1,
or a salt thereof.

4. The compound of claim 1, which is a compound represented by the formula: 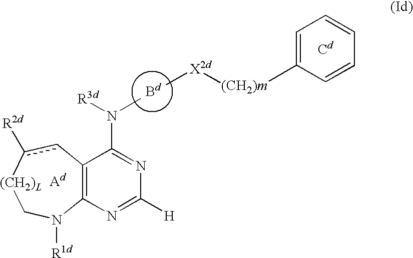
wherein
ring A^{d} is an optionally further substituted nitrogen-containing 7-membered or 8-membered ring,
L is 1 or 2,
formula [symbol] is a single bond or a double bond,
ring B^{d} is an optionally further substituted phenyl group or an optionally further substituted pyridyl group,
ring C^{d} is an optionally substituted phenyl group,
X^{2d} is a group represented by —O—, —S—, —SO—, —SO₂—, —CH₂— or —CO—NR^{5d}— (wherein R^{5d} is a hydrogen atom, or an optionally substituted C₁₋₆ alkyl group),
m is an integer of 0 to 5,
R^{1d} is a hydrogen atom, or an optionally substituted group bonded via a carbon atom or a sulfur atom,
R^{2d} is a hydrogen atom, or an optionally substituted group bonded via a carbon atom, a nitrogen atom, an oxygen atom or a sulfur atom, and
R^{3d} is a hydrogen atom or an optionally substituted aliphatic hydrocarbon group, or R^{3d} is optionally bonded to the carbon atom on ring B^{d} to form an optionally substituted ring structure,
or a salt thereof.

5. The compound of claim 4, wherein
R^{1d} is a hydrogen atom or a C₁₋₆ alkyl group,
R^{2d} is
(i) a C₁₋₆ alkyl group substituted by substituents selected from the group consisting of
(a) hydroxy,
(b) —NH—CO—(CH₂)ₚ—SO₂—C₁₋₆ alkyl (p is an integer of 1 to 6),
(c) —NH—CO—C₁₋₆ alkyl-hydroxy,
(d) —NH—CO—(CH₂)_{p′}—C₁₋₆ alkoxy-C₁₋₆ alkoxy (p′ is an integer of 1 to 6),
(e) C₁₋₆ alkoxyimino substituted by substituents selected from the group consisting of (1) hydroxy, (2) C₁₋₆ alkoxy, (3) di-C₁₋₆ alkylamino, (4) C₁₋₆ alkylsulfonyl and (5) 5- to 8-membered heterocyclic group containing, besides carbon atoms, 1 to 3 heteroatoms selected from the group consisting of nitrogen atom, oxygen atom and sulfur atom,
(f) C₁₋₆ alkylamino having cyano,
(g) C₁₋₆ alkylamino having halogen atom,
(h) C₁₋₆ alkylamino having hydroxy,
(i) C₁₋₆ alkylamino having C₁₋₆ alkoxy,
(j) C₁₋₆ alkylamino having C₁₋₆ alkylsulfonyl optionally having hydroxy,
(k) di-C₁₋₆ alkylamino optionally having 1 or 2 substituents selected from the group consisting of (1) hydroxy, (2) cyano, (3) halogen atom and (4) C₁₋₆ alkylsulfonyl,
(l) C₃₋₇ cycloalkylamino optionally having hydroxy,
(m) 5- to 8-membered heterocyclyl-amino containing, besides carbon atoms, 1 to 3 heteroatoms selected from the group consisting of nitrogen atom, oxygen atom and sulfur atom,
(o) 5- to 8-membered cyclic amino optionally having C₁₋₆ alkoxy or a C₁₋₆ alkylsulfonyl,
(p) N—C₁₋₆ alkyl-N—C₃₋₇ cycloalkylamino optionally having C₁₋₆ alkylsulfonyl,
(q) cyano,
(r) C₁₋₆ alkylamino having C₁₋₆ alkoxy optionally having hydroxy or a C₁₋₆ alkoxy, and
(s) 5- to 8-membered heterocycle containing, besides carbon atoms, 1 to 3 heteroatoms selected from the group consisting of nitrogen atom, oxygen atom and sulfur atom, which is optionally substituted by substituents selected from the group consisting of oxo, C₁₋₆ alkylsulfonyl and C₁₋₆ alkoxy,
(ii) a C₂₋₆ alkenyl group having (a) hydroxy, (b) di-C₁₋₆ alkylamino or (c) C₁₋₆ alkoxy-carbonyl,
(iii) a C₁₋₆ alkoxy-carbonyl group,
(iv) a group represented by —CO—NR^{a}R^{b}
wherein
R^{a} is a hydrogen atom or a C₁₋₆ alkyl group, and
R^{b} is
(a) a C₁₋₆ alkyl group optionally substituted by 1 or 2 substituents selected from the group consisting of
(1) hydroxy,
(2) amino,
(3) C₁₋₆ alkylamino having hydroxy,
(4) C₁₋₆ alkylamino having C₁₋₆ alkoxy,
(5) cyano,
(6) amino mono- or di-substituted by C₁₋₆ alkyl optionally having hydroxy,
(7) C₁₋₆ alkyl-carbonylamino,
(8) C₁₋₆ alkoxy,
(9) C₁₋₆ alkoxy having hydroxy,
(10) C₁₋₆ alkoxy having C₁₋₆ alkoxy,
(11) C₁₋₆ alkoxy having hydroxy and C₁₋₆ alkoxy,
(12) C₁₋₆ alkoxy having C₁₋₆ alkylsulfonyl,
(13) C₁₋₆ alkoxy having cyano,
(14) C₁₋₆ alkoxy-carbonyl,
(15) C₁₋₆ alkylsulfonyl optionally having hydroxy or C₁₋₆ alkoxy,
(16) 5- to 8-membered heterocyclyl-sulfonyl containing, besides carbon atoms, 1 to 3 heteroatoms selected from the group consisting of nitrogen atom, oxygen atom and sulfur atom,
(17) C₆₋₁₈ arylsulfonyl,
(18) 5- to 8-membered heterocycle containing, besides carbon atoms, 1 to 3 heteroatoms selected from the group consisting of nitrogen atom, oxygen atom and sulfur atom, which optionally has 1 or 2 substituents selected from the group consisting of hydroxy, C₁₋₆ alkyl, C₆₋₁₈ aryl and C₆₋₁₈ aryl-C₁₋₆ alkyl,
(19) C₃₋₇ cycloalkyl optionally having hydroxy, and
(20) C₆₋₁₈ aryl optionally having 1 or 2 halogens,
(b) a C₂₋₆ alkenyl group,
(c) a C₃₋₇ cycloalkyl group optionally having hydroxy,
(d) a C₁₋₆ alkoxy group, or
(e) a 5- to 8-membered heterocyclic group containing, besides carbon atoms, 1 to 3 heteroatoms selected from the group consisting of nitrogen atom, oxygen atom and sulfur atom, which optionally has 1 or 2 substituents selected from the group consisting of hydroxy, C₁₋₆ alkyl, C₆₋₁₈ aryl and C₆₋₁₈ aryl-C₁₋₆ alkyl,
(v) a 5- to 8-membered cyclic amino-carbonyl group optionally having substituents selected from the group consisting of
(a) hydroxy,
(b) C₁₋₆ alkylsulfonyl, and
(c) C₁₋₆ alkyl optionally having C₁₋₆ alkylsulfonyl,
(vi) a carboxy group,
(vii) an amino group optionally substituted by C₁₋₆ alkoxy-carbonyl optionally having C₁₋₆ alkylsulfonyl, or
(viii) a 5- to 8-membered heterocyclic group containing, besides carbon atoms, 1 to 3 heteroatoms selected from the group consisting of nitrogen atom, oxygen atom and sulfur atom, which optionally has C₁₋₆ alkyl,
R^{3d} is a hydrogen atom,
ring B^{d} is a phenyl group optionally further substituted by C₁₋₆ alkyl group or halogen atom, or a pyridyl group optionally further substituted by C₁₋₆ alkyl group or halogen atom,
ring C^{d} is a phenyl group optionally substituted by substituents selected from the group consisting of
(i) optionally halogenated C₁₋₆ alkyl,
(ii) C₁₋₆ alkoxy optionally having halogen atom or C₃₋₇ cycloalkyl,
(iii) C₁₋₆ alkyl-carbamoyl optionally having hydroxy,
(iv) halogen atom,
(v) cyano,
(vi) C₁₋₆ alkylthio optionally having halogen atom,
(vii) C₁₋₆ alkylsulfinyl optionally having halogen atom, and
(viii) C₁₋₆ alkylsulfonyl optionally having halogen atom or C₃₋₇ cycloalkyl,
X^{2d} is a group represented by —O— or —S—, and
m is 0 or 1.

6. The compound of claim 1, which is a compound represented by the formula: 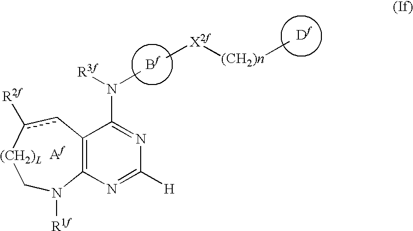
wherein
ring A^{f} is an optionally further substituted nitrogen-containing 7-membered or 8-membered ring,
L is 1 or 2,
formula [symbol] is a single bond or a double bond,
ring B^{f} is an optionally further substituted phenyl group or an optionally further substituted pyridyl group,
ring D^{f} is an optionally substituted aromatic heterocyclic group,
X^{2f} is a group represented by —O—, —S—, —SO—, —SO₂—, —CH₂— or —CO—NR^{5f}— (wherein R^{5f} is a hydrogen atom, or an optionally substituted C₁₋₆ alkyl group),
n is an integer of 0 to 5,
R^{1f} is a hydrogen atom, or an optionally substituted group bonded via a carbon atom or a sulfur atom,
R^{2f} is a hydrogen atom, or an optionally substituted group bonded via a carbon atom, a nitrogen atom, an oxygen atom or a sulfur atom, and
R^{3f} is a hydrogen atom or an optionally substituted aliphatic hydrocarbon group, or R^{3f} is optionally bonded to the carbon atom on ring B^{f} to form an optionally substituted ring structure,
or a salt thereof.

7. The compound of claim 6, wherein
L is 1,
R^{1f} is a hydrogen atom or a C₁₋₆ alkyl group,
R^{2f} is
(i) a C₁₋₆ alkyl group optionally having hydroxy,
(ii) a C₁₋₆ alkoxy-carbonyl group,
(iii) a group represented by —CO—NR^{c}R^{d}
wherein
R^{c} is a hydrogen atom, and
R^{d} is
(a) a C₁₋₆ alkyl group optionally substituted by substituents selected from the group consisting of (1) C₁₋₆ alkoxy, (2) C₁₋₆ alkoxy optionally having substituents selected from the group consisting of (1′) hydroxy, (2′) cyano and (3′) C₁₋₆ alkoxy, and (3) C₁₋₆ alkylsulfonyl optionally having hydroxy,
(b) a C₃₋₇ cycloalkyl group, or
(c) a C₁₋₆ alkoxy group optionally substituted by C₁₋₆ alkylsulfonyl,
(iv) a 5- to 8-membered cyclic amino-carbonyl group optionally substituted by substituents selected from the group consisting of
(a) hydroxy, and
(b) C₁₋₆ alkyl optionally having hydroxy, or
(v) a carboxy group,
R^{3f} is a hydrogen atom,
ring B^{f} is a phenyl group optionally further substituted by C₁₋₆ alkyl group,
X^{f} is a group represented by —O—,
n is 0, and
ring D^{f} is a 5- or 6-membered monocyclic aromatic heterocycle containing, besides carbon atoms, 1 to 3 heteroatoms selected from the group consisting of nitrogen atom, oxygen atom and sulfur atom, which is optionally substituted by C₁₋₆ alkyl group.

8. The compound of claim 1, which is a compound represented by the formula: 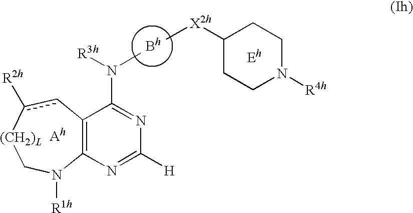
wherein
ring A^{h} is an optionally further substituted nitrogen-containing 7-membered or 8-membered ring,
L is 1 or 2,
formula [symbol] is a single bond or a double bond,
ring B^{h} is an optionally further substituted phenyl group or an optionally further substituted pyridyl group,
ring E^{h} is an optionally further substituted piperidyl group,
X^{2h} is a group represented by —O—, —S—, —SO—, —SO₂—, —CH₂— or —CO—NR^{5h}— (wherein R^{5h} is a hydrogen atom or an optionally substituted C₁₋₆ alkyl group),
R^{1h} is a hydrogen atom, or an optionally substituted group bonded via a carbon atom or a sulfur atom,
R^{2h} is a hydrogen atom, or an optionally substituted group bonded via a carbon atom, a nitrogen atom, an oxygen atom or a sulfur atom,
R^{3h} is a hydrogen atom, or an optionally substituted aliphatic hydrocarbon group, or R^{3h} is optionally bonded to the carbon atom on ring B^{h} to form an optionally substituted ring structure, and
R^{4h} is a hydrogen atom or an acyl group,
or a salt thereof.

9. The compound of claim 8, wherein
L is 1,
R^{1h} is a hydrogen atom,
R^{2h} is
(i) a C₁₋₆ alkyl group optionally substituted by hydroxy,
(ii) a C₁₋₆ alkoxy-carbonyl group,
(iii) a group represented by —CO—NR^{e}R^{f}
wherein
R^{e} is a hydrogen atom or a C₁₋₆ alkyl group,
R^{f} is
(a) a C₁₋₆ alkyl group optionally substituted by 1 or 2 substituents selected from the group consisting of
(1) hydroxy,
(2) amino,
(3) cyano,
(4) amino mono- or di-substituted by C₁₋₆ alkyl optionally having hydroxy,
(5) C₁₋₆ alkyl-carbonylamino,
(6) C₁₋₆ alkoxy optionally having hydroxy,
(7) C₁₋₆ alkoxy-carbonyl,
(8) C₁₋₆ alkylsulfonyl,
(9) 5- to 8-membered heterocyclyl-sulfonyl containing, besides carbon atoms, 1 to 3 heteroatoms selected from the group consisting of nitrogen atom, oxygen atom and sulfur atom,
(10) 5- to 8-membered heterocyclic group containing, besides carbon atoms, 1 to 3 heteroatoms selected from the group consisting of nitrogen atom, oxygen atom and sulfur atom, which optionally has 1 to 2 substituents selected from the group consisting of C₁₋₆ alkyl, C₆₋₁₈ aryl and C₆₋₁₈ aryl-C₁₋₆ alkyl,
(11) C₆₋₁₈ aryl-sulfonyl, and
(12) C₆₋₁₈ aryl group optionally having 1 to 2 halogens,
(b) a C₃₋₇ cycloalkyl group,
(c) a C₁₋₆ alkoxy group, or
(d) a 5- to 8-membered heterocyclic group containing, besides carbon atoms, 1 to 3 heteroatoms selected from the group consisting of nitrogen atom, oxygen atom and sulfur atom, which optionally has C₁₋₆ alkyl or C₆₋₁₈ aryl-C₁₋₆ alkyl, or
(iv) a 5- to 8-membered cyclic amino-carbonyl group optionally substituted by substituents selected from the group consisting of
(a) hydroxy,
(b) C₁₋₆ alkylsulfonyl, and
(c) C₁₋₆ alkyl optionally having C₁₋₆ alkylsulfonyl,
R^{3h} is a hydrogen atom,
ring B^{h} is a phenyl group optionally further substituted by halogen atom,
X^{2h} is a group represented by —O—, and
R^{4h} is (1) a C₃₋₇ cycloalkyl-carbonyl group, (2) a C₁₋₆ alkoxy-carbonyl group, or (3) a 5- to 8-membered heterocyclyl-carbonyl group containing, besides carbon atoms, 1 to 3 heteroatoms selected from the group consisting of nitrogen atom, oxygen atom and sulfur atom, which optionally has 1 or 2 C₁₋₆ alkyls.

10. A compound selected from the following, or a salt thereof;
4-({3-chloro-4-[3-(trifluoromethyl)phenoxy]phenyl}amino)-N-[3-(1H-imidazol-1-yl)propyl]-8,9-dihydro-7H-pyrimido[4,5-b]azepin-6-carboxamide,
4-({3-chloro-4-[3-(trifluoromethyl)phenoxy]phenyl}amino)-N-[2-(2-hydroxyethoxy)ethyl]-8,9-dihydro-7H-pyrimido[4,5-b]azepin-6-carboxamide,
4-({3-chloro-4-[3-(trifluoromethyl)phenoxy]phenyl}amino)-N-[2-(2-methoxyethoxy)ethyl]-8,9-dihydro-7H-pyrimido[4,5-b]azepin-6-carboxamide,
methyl 4-[(4-{3-[(tert-butylamino)carbonyl]phenoxy}-3-chlorophenyl)amino]-8,9-dihydro-7H-pyrimido[4,5-b]azepine-6-carboxylate,
4-({3-chloro-4-[3-(trifluoromethyl)phenoxy]phenyl}amino)-N-(2,3-dihydroxypropyl)-8,9-dihydro-7H-pyrimido[4,5-b]azepine-6-carboxamide,
4-{[3-chloro-4-(3-chlorophenoxy)phenyl]amino}-N-[2-(2-hydroxyethoxy)ethyl]-8,9-dihydro-7H-pyrimido[4,5-b]azepin-6-carboxamide,
tert-butyl 4-(2-chloro-4-{[6-(hydroxymethyl)-8,9-dihydro-7H-pyrimido[4,5-b]azepin-4-yl]amino}phenoxy)piperidine-1-carboxylate,
4-({3-chloro-4-[3-(trifluoromethyl)phenoxy]phenyl}amino)-N-{2-[(2-hydroxyethyl)sulfonyl]ethyl}-8,9-dihydro-7H-pyrimido[4,5-b]azepin-6-carboxamide,
4-({3-chloro-4-[3-(trifluoromethyl)phenoxy]phenyl}amino)-N-{2-[(2-hydroxyethyl)sulfonyl]-1,1-dimethylethyl}-8,9-dihydro-7H-pyrimido[4,5-b]azepin-6-carboxamide,
N-(tert-butyl)-3-[2-chloro-4-({6-[({2-[(2-hydroxyethyl)sulfonyl]-1,1-dimethylethyl}amino)methyl]-8,9-dihydro-7H-pyrimido[4,5-b]azepin-4-yl}amino)phenoxy]benzamide,
methyl 4-[(6-{3-[(tert-butylamino)carbonyl]phenoxy}-5-chloropyridin-3-yl)amino]-8,9-dihydro-7H-pyrimido[4,5-b]azepin-6-carboxylate,
4-[(3-chloro-4-{3-[(cyclopropylmethyl)sulfonyl]phenoxy}phenyl)amino]-N-{2-[(2-hydroxyethyl)sulfonyl]-1,1-dimethylethyl}-8,9-dihydro-7H-pyrimido[4,5-b]azepin-6-carboxamide,
N-(tert-butyl)-3-{2-chloro-4-[(6-{[(2-hydroxy ethoxy)imino]methyl}-8,9-dihydro-7H-pyrimido[4,5-b]azepin-4-yl)amino]phenoxy}benzamide,
N-(tert-butyl)-3-{2-chloro-4-[(6-{[(2-fluoroethyl)amino]methyl}-8,9-dihydro-7H-pyrimido[4,5-b]azepin-4-yl)amino]phenoxy}benzamide,
N-(tert-butyl)-3-(2-chloro-4-{[6-({methyl[2-(methylsulfonyl)ethyl]amino}methyl)-8,9-dihydro-7H-pyrimido[4,5-b]azepin-4-yl]amino}phenoxy)benzamide, or
ethyl (2E)-3-{4-[(4-{3-[(tert-butylamino)carbonyl]phenoxy}-3-chlorophenyl)amino]-8,9-dihydro-7H-pyrimido[4,5-b]azepin-6-yl}acrylate.

11. A prodrug of a compound represented by the formula: 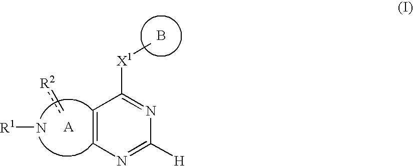
wherein
ring A is an optionally further substituted nitrogen-containing 7-membered or 8-membered ring,
ring B is an optionally substituted aryl group or an optionally substituted heteroaryl group,
X¹ is a group represented by —NR³—Y¹—, —O—, —S—, —SO—, —SO₂— or —CHR³—
wherein
R³ is a hydrogen atom or an optionally substituted aliphatic hydrocarbon group, or
R³ is optionally bonded to the carbon atom on ring B to form an optionally substituted ring structure, and
Y¹ is a bond or an optionally substituted C₁₋₄ alkylene,
R¹ is a hydrogen atom, or an optionally substituted group bonded via a carbon atom or a sulfur atom,
formula [symbol] is a single bond or a double bond, and
R² is a hydrogen atom, or an optionally substituted group bonded via a carbon atom, a nitrogen atom, an oxygen atom or a sulfur atom when [symbol] R² is —R², and an oxo group, an optionally substituted alkylidene group or an optionally substituted imino group when [symbol] R² is ═R², or
R¹ and R², or R² and R³ are optionally bonded to each other to form an optionally substituted ring structure.

12. A pharmaceutical agent comprising a compound represented by the formula: 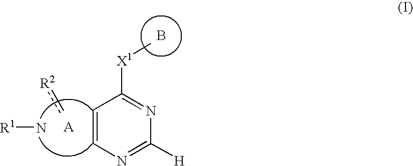
wherein
ring A is an optionally further substituted nitrogen-containing 7-membered or 8-membered ring,
ring B is an optionally substituted aryl group or an optionally substituted heteroaryl group,
X¹ is a group represented by —NR³—Y¹—, —O—, —S—, —SO—, —SO₂— or —CHR³—
wherein
R³ is a hydrogen atom or an optionally substituted aliphatic hydrocarbon group, or
R³ is optionally bonded to the carbon atom on ring B to form an optionally substituted ring structure, and
Y¹ is a bond or an optionally substituted C₁₋₄ alkylene,
R¹ is a hydrogen atom, or an optionally substituted group bonded via a carbon atom or a sulfur atom,
formula [symbol] is a single bond or a double bond, and
R² is a hydrogen atom, or an optionally substituted group bonded via a carbon atom, a nitrogen atom, an oxygen atom or sulfur atom when [symbol] R² is —R², and an oxo group, an optionally substituted alkylidene group or an optionally substituted imino group when [symbol] R² is ═R², or
R¹ and R², or R² and R³ are optionally bonded to each other to form an optionally substituted ring structure,
or a salt thereof, or a prodrug thereof.

13. The pharmaceutical agent of claim 12, which is a tyrosine kinase inhibitor.

14. The pharmaceutical agent of claim 12, which is an agent for the prophylaxis or treatment of cancer.

15. The pharmaceutical agent of claim 14, wherein the cancer is breast cancer, ovarian cancer, colorectal cancer, gastric cancer, esophagus cancer, prostate cancer, lung cancer, pancreatic cancer or renal cancer.

16. A method for the prophylaxis or treatment of cancer, which comprises administering an effective amount of a compound represented by the formula: 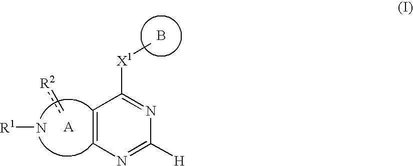
wherein
ring A is an optionally further substituted nitrogen-containing 7-membered or 8-membered ring,
ring B is an optionally substituted aryl group or an optionally substituted heteroaryl group,
X¹ is a group represented by —NR³—Y¹—, —O—, —S—, —SO—, —SO₂— or —CHR³—
wherein
R³ is a hydrogen atom or an optionally substituted aliphatic hydrocarbon group, or
R³ is optionally bonded to the carbon atom on ring B to form an optionally substituted ring structure, and
Y¹ is a bond or an optionally substituted C₁₋₄ alkylene,
R¹ is a hydrogen atom, or an optionally substituted group bonded via a carbon atom or a sulfur atom,
formula [symbol] is a single bond or a double bond, and
R² is a hydrogen atom, or an optionally substituted group bonded via a carbon atom, a nitrogen atom, an oxygen atom or a sulfur atom when [symbol] R² is —R², and an oxo group, an optionally substituted alkylidene group or an optionally substituted imino group when [symbol] R² is ═R², or
R¹ and R², or R² and R³ are optionally bonded to each other to form an optionally substituted ring structure,
or a salt thereof, or a prodrug thereof, to the mammal.

17. (canceled)


SEQUENCE LISTING (4 sequences)

SEQ ID NO: 1 - DNA, 66 bp, Artificial
  primer for cloning human HER2 gene
aattaagtcgacatggactacaaagacgatgacgacaagcgacggcagcagaagatccggaagtac

SEQ ID NO: 2 - DNA, 42 bp, Artificial
  primer for cloning human HER2 gene
aattaagcatgctcacactggcacgtccagacccaggtactc

SEQ ID NO: 3 - DNA, 66 bp, Artificial
  primer for cloning human EGFR gene
aattaagtcgacatggactacaaagacgatgacgaccgaaggcgccacatcgttcggaagcgcacg

SEQ ID NO: 4 - DNA, 42 bp, Artificial
  primer for cloning human EGFR gene
aattaagcatgctcatgctccaataaattcactgctttgtgg